(19) United States
(12) Patent Application Publication
(10) Pub. No.: US 2010/0247580 A1
(43) Pub. Date: Sep. 30, 2010

(54) Method for Classifying Cancer Patients as Responder or Non-Responder to Immunotherapy

(76) Inventors: Thierry Coche, Rixensart (BE); Swann Gaulis, Rixensart (BE); Jamila Louahed, Rixensart (BE)

Correspondence Address: GLAXOSMITHKLINE;GLOBAL PATENTS, C.2111F, FIVE MOORE DR., PO BOX 13398, RESEARCH TRIANGLE PARK, NC, 27709-3398, US

(21) Appl. No.: 12/744,612

(22) PCT Filed: Nov. 27, 2008
(86) PCT No.: PCT/EP08/66357

§ 371 Date: May 25, 2010

Related U.S. Application Data
(60) Provisional application No. 60/991,458, filed on Nov. 30, 2007.

(51) Int. Cl. A61K 39/00; C12Q 1/68; A61P 35/04

(52) U.S. Cl. 424/277.1; 435/6

(57) ABSTRACT

The present invention relates to gene expression profiles; methods for classifying patients; microarrays; and treatment of population of patients selected through use of methods and microarrays as described herein.

### FIELD OF THE INVENTION

The present invention relates to gene expression profiles; methods for classifying patients; microarrays; and treatment of populations of patients selected through use of methods and microarrays as described herein.

### BACKGROUND

Melanomas are tumours originating from melanocyte cells in the epidermis. Patients with malignant melanoma in distant metastasis (stage 1V according to the American Joint Commission on Cancer (AJCC) classification) have a median survival time of one year, with a long-term survival rate of only 5%. Even standard chemotherapy for stage 1V melanoma has therapeutic response rates of only 8-25%, but with no effect on overall survival. Patients with regional metastases (stage III) have a median survival of two to three years with very low chance of long-term survival, even after an adequate surgical control of the primary and regional metastases (Balch et al., 1992). Most patients with stage I to III melanoma have their tumour removed surgically, but these patients maintain a substantial risk of relapse.

There are two types of lung cancer: non-small cell lung cancer (NSCLC) and small cell lung cancer (SCLC). The names simply describe the type of cell found in the tumours. NSCLC includes squamous-cell carcinoma, adenocarcinoma, and large-cell carcinoma and accounts for around 80% of lung cancers. NSCLC is hard to cure and treatments available tend to have the aim of prolonging life, as far as possible, and relieving symptoms of disease. NSCLC is the most common type of lung cancer and is associated with poor outcome. Of all NSCLC patients, only about 25% have loco-regional disease at the time of diagnosis and are still amenable to surgical excision (stages IB, IIA or IIB according to the AJCC classification). However, more than 50% of these patients will relapse within the two years following the complete surgical resection. There is therefore a need to provide better treatment for these patients.

Traditional chemotherapy is based on administering toxic substances to the patient and relying, in part, on the aggressive uptake of the toxic agent by the tumour/cancer cells. These toxic substances adversely affect the patient's immune system, leaving the individual physically weakened and susceptible to infection.

It is known that not all patients with cancer respond to current cancer treatments. It is thought that only 30% or less of persons suffering from a cancer will respond to any given treatment. The cancers that do not respond to treatment are described as resistant. In many instances there have not been reliable methods for establishing if the patients will respond to treatment. However, administering treatment to patients who are both responders and non-responders because they cannot be differentiated is an inefficient use of resources and, even worse, can be damaging to the patient because, as discussed already, many cancer treatments have significant side effects, such as severe immunosuppression, emesis and/or alopecia. It is thought that in a number of cases patients receive treatment, when it is not necessary or when it will not be effective.

Cells, including cancer/tumour cells, express many hundreds even thousands of genes.

A large amount of work has been done in recent times to assist in the diagnosis and prognosis of cancer patients, for example to identify those patients who do not require further treatment because they have no risk of metastasis, recurrence or progression of the disease.

Immune-mediated treatments based on antigens, peptides, DNA and the like are under investigation. The strategy behind many of these treatments is to stimulate a patient's own immune system into fighting the cancer. One immune mediated treatment is described as Antigen-Specific Cancer Immunotherapy (ASCI).

### STATEMENT OF THE INVENTION

In one embodiment, the present invention provides a method for classifying a patient as a responder or non-responder to therapy, comprising measuring, in a patient-derived sample, the gene product of at least one gene selected from the genes listed in Table 1, 2, 4, 5 or 6.

By therapy is meant chemotherapy or radiotherapy, or antigen-specific immunotherapy (ASCI) as described herein, for example MAGE antigen specific cancer immunotherapy as described herein, or administration of a composition, vaccine or immunogenic composition as described herein. Therapy may additionally mean another form of therapy, for example passive immunotherapy or the targeting of tumour tissue or cells/receptors therein through other therapeutic means.

In one embodiment there is provided a method comprising the step of: applying a statistical analysis to the results of measuring differential expression of one or more of the genes in Table 1, 2, 4, 5 or 6 or a combination thereof in a biological sample for differentiating responders and/or non-responders to an appropriate immunotherapy. The method may comprise the steps:
a) measuring differential expression of one or more of the genes in Table 1, 2, 4, 5 or 6 or a combination thereof in a biological sample, and
b) applying a statistical analysis to the results of step a) to differentiate a responders and/or non-responder to an appropriate immunotherapy.

In one embodiment, the statistical analysis is employed on data generated in relation to differential regulation of substantially all the genes in Table 1, Table 2, Table 4, Table 5 or Table 6. The statistical analysis employed may be a T-test, such as a Baldi analysis.

In one embodiment there is provided a use of a gene list of substantially all the genes in Table 1, Table 2, Table 4, Table 5 or Table 6 or a combination thereof to perform an analysis of whether a patient will be a likely responder or non-responder to an immunotherapy, such as cancer immunotherapy. In a further embodiment there is provided use (or a method of use) of a gene list of substantially all the genes in Table 1, Table 2, Table 4, Table 5 or Table 6 or a combination thereof or data generated therefrom for treatment, particularly for the treatment of cancer. The method or use may further comprise administering a therapeutically effective amount of an appropriate immunotherapy, such as a cancer immunotherapy, particularly MAGE.

In one embodiment, the present invention provides a method for the detection of a gene signature, indicative of a responder or non-responder to therapy, such as cancer immunotherapy, employing novel gene(s)/gene lists, which method comprises the step of:
a) applying a statistical analysis to the results of measuring in a biological sample differential expression of one or more of the genes in Table 1, 2, 4, 5 or 6 or a combination thereof for differentiating a responder and/or non-responder to an appropriate immunotherapy.

In one aspect the invention provides a method for the detection of a gene signature, indicative of a responder or non-responder to therapy, such as cancer immunotherapy, employing novel gene(s)/gene lists, which method comprises the steps of:
a) measuring differential expression of one or more of the genes in Table 1, 2, 4, 5 or 6 or a combination thereof in a biological sample, and
b) applying a statistical analysis to differentiate responders and/or non-responders to an appropriate immunotherapy.

Whilst not wishing to be bound by theory it is hypothesised that the gene signature identified in at least Tables 1 and 2 is in fact indicative of an immune/inflammatory response, such as a T cell infiltration/activation response in the patients who are designated as responders, for example, the signature may represent a T-cell activation marker. The presence of this response is thought to assist the patient's body to fight the disease, such as cancer, after administration of a therapy, for example an immunotherapy, thereby rendering a patient more responsive to the therapy.

Thus the signatures of the present invention do not generally focus on markers/genes specifically associated with the diagnosis and/or prognosis of cancer, such as oncogenes, but rather is predictive of whether the patient will respond to an appropriate immunotherapy, such as cancer immunotherapy.

Thus in one aspect the invention provides a method of identifying whether a cancer patient will be a responder or non-responder to therapy, such as cancer immunotherapy, the method comprising:
(a) analysing a sample comprising mRNA or cDNA, or fragments thereof, expressed by genes of cancerous cells for differential expression of one or more genes selected from the group comprising or consisting of genes listed in Table 1, 2, 4, 5, 6 or a combination thereof; and
(b) characterising a patient as a responder or a non-responder based on the results of step 1.

The genes may be selected from genes in Tables 1, 2, 4, 5 and/or 6.

In one embodiment the invention employs one or more (such as substantially all) the genes listed in Tables 1, 2, or 5 respectively.

In one embodiment the invention employs one or more, for example all or substantially all, of the genes listed in Table 1.

In one embodiment the invention employs one or more, for example all or substantially all, of the genes listed in Table 2.

In one embodiment the invention employs one or more, for example all or substantially all, of the genes listed in Table 4.

In one embodiment the invention employs one or more, for example all or substantially all, of the genes listed in Table 5.

In one embodiment the invention employs one or more, for example all or substantially all, of the genes listed in Table 6.

Substantially all in the context of the gene lists will be at least 90%, such a 95%, particularly 96, 97, 98 or 99% of the genes in the given list.

In one aspect the invention is employed in a metastatic setting.

Differential expression in the context of the present invention means the gene is upregulated or downregulated in comparison to its normal expression. Statistical methods for calculating gene differentiation are discussed below.

Whilst not wishing to be bound by theory it is thought that at least the genes in Tables 1, 2 and 5 are strongly differentially expressed, which may render certain aspects of the invention particularly advantageous.

If a gene is always upregulated or always down regulated in patients that are deemed to be responders (or alternatively non-responders) then this single gene can be used to establish if the patient is a responder or a non-responder once a threshold is established and provided the separation of the two groups is adequate.

In one aspect the invention provides a gene profile for identifying a responder comprising one or more of said genes wherein 50, 60, 70, 75, 80, 85, 90, 95, 99 or 100% of the genes are upregulated. In contrast, in non-responders, the gene/genes is/are not upregulated or is/are down regulated.

In one aspect the present invention provides a therapy or cancer immunotherapy for the treatment of melanoma, lung cancer for example NSCLC, bladder cancer, neck cancer, colon cancer, breast cancer, oesophageal carcinoma and/or prostate cancer, such as lung cancer and/or melanoma, in particular melanoma

### BRIEF DESCRIPTION OF THE FIGURES

FIG. 1—Performance of the clinical outcome prediction of first data stratification under increasing number of differentially expressed probe sets.

FIG. 2—Performance of the clinical outcome prediction of second data stratification under increasing number of differentially expressed probe sets.

FIG. 3—Performance of clinical outcome predictions versus threshold level

FIG. 4—Multivariate analysis (Correspondence analysis) on the Baldi generated list.

FIG. 5—Axes that correlate with the segregation of the samples and genes by gender and response.

FIG. 6—Figure showing that genes of interest for classification of patients are those located close to the Response axis.

FIG. 7—the training set for Table 5 represented as an index.

FIG. 8—shows the gene list of Table 5 used to predict on a small number of samples.

FIG. 9—Appendix A.

FIG. 10—Appendix B.

FIG. 11—protein D—MAGE-A3 fusion protein.

FIG. 12—protein D partner protein.

### DETAILED DESCRIPTION

### Sequence Identifiers and Tables

The following sequence identifiers are included in the sequence listing:

SEQ ID NO:1-18 Probe set target sequences for 18 PS gene list

SEQ ID NO:19-268 Probe set target sequences for 250 PS gene list

SEQ ID NO:269-368 Probe set target sequences for 100 PS gene list

SEQ ID NO:369-473 Probe set target sequences for 105 PS gene list

SEQ ID NO:474-507 Probe set target sequences for 34 PS gene list

SEQ ID NO: 508-705: 18 PS gene list probe sequences

SEQ ID NO: 706-3452: 250 PS gene list probe sequences

SEQ ID NO: 3453-4557: 100 PS gene list probe sequences

SEQ ID NO: 4558-5717: 105 PS gene list probe sequences

SEQ ID NO: 5718-6091: 34 PS gene list probe sequences

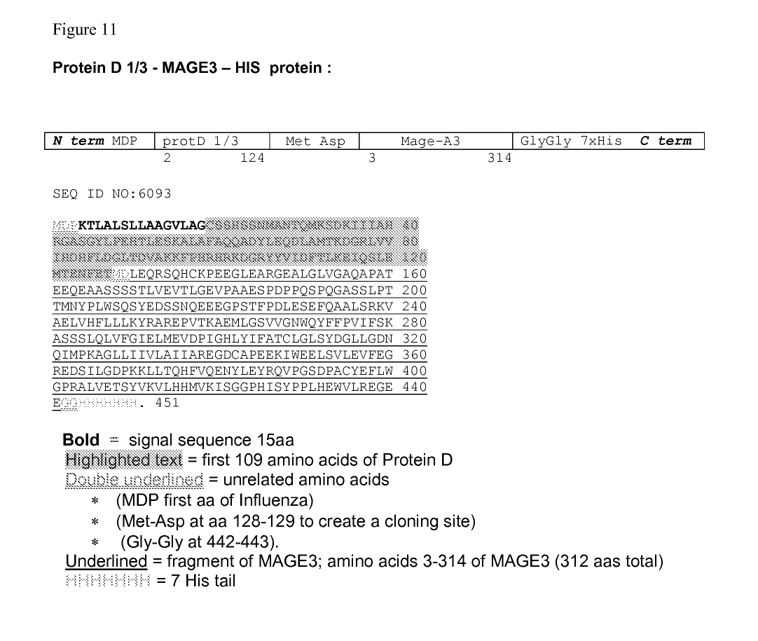

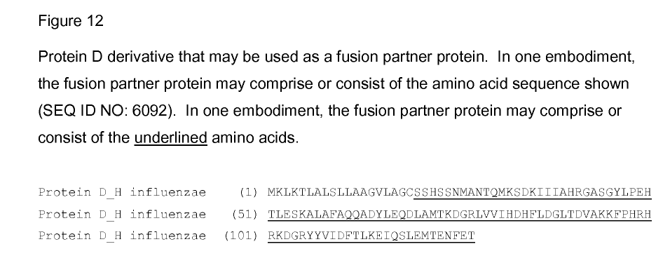

SEQ ID NO: 6092 Protein D fusion partner protein (FIG. 12)

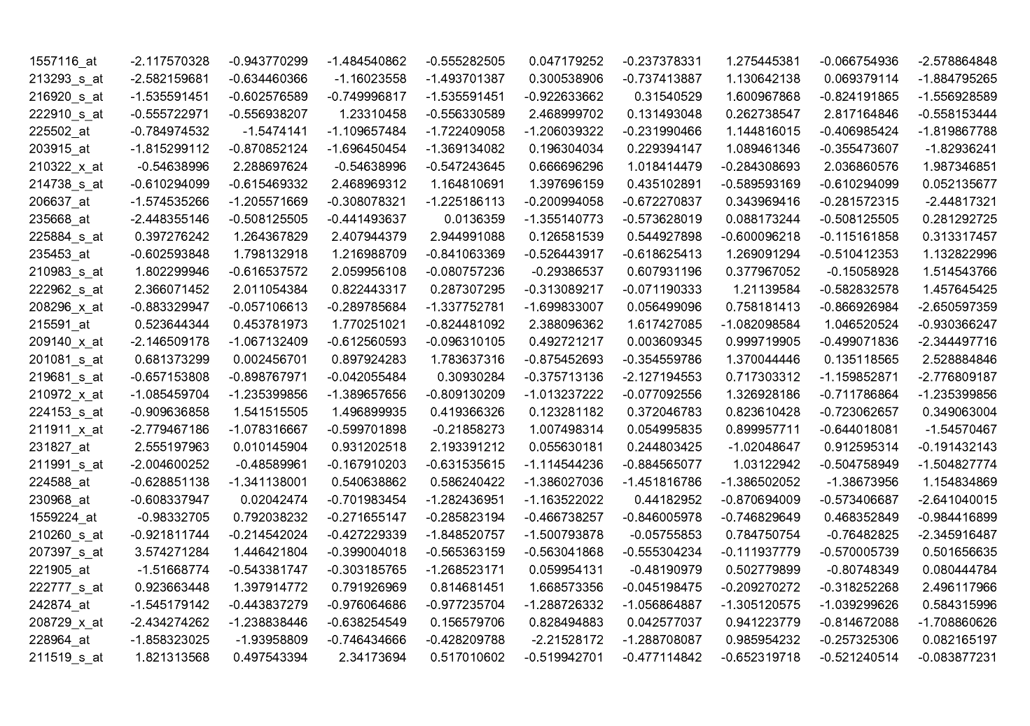

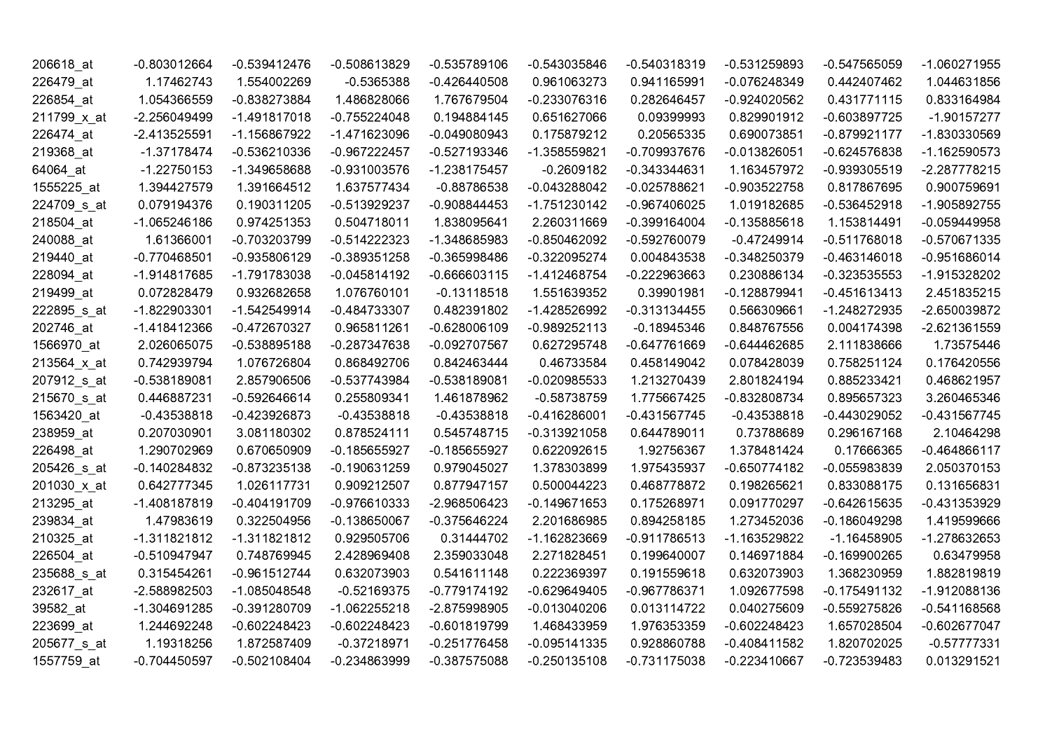

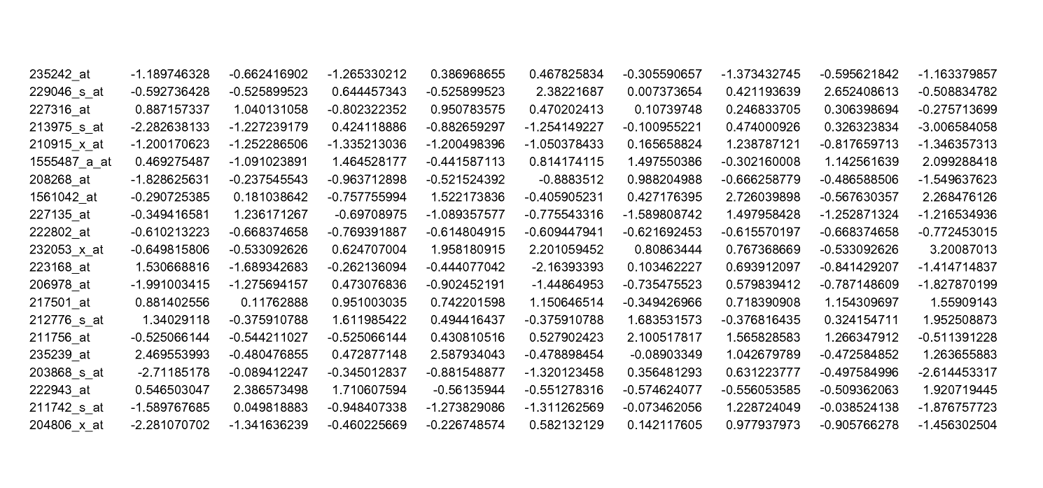

SEQ ID NO: 6093 Protein D—MAGE-A3 fusion protein (FIG. 11)

SEQ ID NO: 6094-6101 MAGE peptide sequences

SEQ ID NO: 6102-6106 CpG oligonucleotide sequences

As described in greater detail elsewhere, the following tables are set forth at the end of the description:
Table 1-18 PS gene list
Table 2—250 PS gene list
Table 3—Median and Standard Deviation Values for 250 PS gene list
Table 4—100 PS gene list
Table 5—105 PS gene list
Table 6—34 PS gene list
Table 7—Value of PS ID in the metagene for the 100 PS gene list
Table 8—Value of PS ID in the metagene for the 105 PS gene list
Table 9—Probe set target sequences for 18 PS gene list
Table 10—Probe set target sequences for 250 PS gene list
Table 11—Probe set target sequences for 100 PS gene list
Table 12—Probe set target sequences for 105 PS gene list
Table 13—Probe set target sequences for 34 PS gene list
Table 14—Sequences of probe sets—18 PS gene list
Table 15—Sequences of probe sets—250 PS gene list
Table 16—Sequences of probe sets—100 PS gene list
Table 17—Sequences of probe sets—105 PS gene list
Table 18—Sequences of probe sets—34 PS gene list

### Predictive Gene Profiles

“Responder” in the context of the present invention includes persons where the cancer/tumour(s) is eradicated, reduced or improved (Mixed Responder or Partial Responder) or simply stabilised such that the disease is not progressing (“Stable Disease”) after treatment. In responders where the cancer is stabilised then the period of stabilisation is such that the quality of life and/or patients life expectancy is increased (for example stable disease for more than 6, 7, 8, 9, 10, 11, 12, 13, 14, 15, 16, 17, 18, 19, 20, 21, 22, 23, 24 or more months) in comparison to a patient that does not receive treatment.

“Partial clinical responder” or “Partial Responder” in respect of cancer is wherein all of the tumours/cancers respond to treatment to some extent, for example where said cancer is reduced by 30, 40, 50, 60% or more.

“Mixed clinical responder” or “Mixed Responder” in respect of cancer is defined as wherein some of the tumours/cancers respond to treatment and others remain unchanged or progress.

Optionally the characterisation of the patient as a responder or non-responder can be performed by reference to a “standard” or a training set. The standard may be the profile of a person/patient who is known to be a responder or non-responder or alternatively may be a numerical value. Such pre-determined standards may be provided in any suitable form, such as a printed list or diagram, computer software program, or other media.

Training set in the context of the present specification is intended to refer to a group of samples for which the clinical results can be correlated with the gene profile and can be employed for training an appropriate statistical model/programme to identify responders and/or non-responder for new samples.

In one aspect to the invention the statistical analysis employed is a signal to noise classifier or a T-test such as Baldi analysis.

In another aspect the statistical analysis is a Pearson's Correlation Coefficient and/or Linear Discriminant

In another aspect the statistical analysis is Supervised Principal Components Analysis (SPCA).

In one aspect the statistical analysis is performed by reference to a “standard” or training set. The standard may be the profile of a person/patient who has a known clinical outcome or alternatively may be a numerical value. Such pre-determined standards may be provided in any suitable form, such as a printed list or diagram, computer software program, or other media. The gene lists in Table 1 and 2 were generated by correlating clinical outcome with gene profiles. A training set is then used to predict the classification for new samples.

In one aspect a mathematical model/algorithm/statistical method is employed to characterise the patient as responder or non-responder.

In one embodiment the responder and non-responder are defined by reference to the Time To Treatment Failure (TTTF), which is a continuous variable and may, for example, be measured in months. Where the time to treatment failure variable is large, for example when the patient does not relapse or show any disease progression for several months, the patient may be considered to be a responder. Where the time to treatment failure variable is small, for example the patient shows disease progression within three, four, five or six months, then patient may be considered to be a non-responder.

Treatment failure can be defined as where the patient does not fall with the definition of responder, partial responder or stable disease as defined herein. In one embodiment, using this approach, the mixed responders may be grouped with the responders.

However, in some embodiments, mixed responders may be grouped with the non-responders.

In one aspect non-responders may be defined as those with a TTTF of 6 months or less.

In one aspect the responders may be defined as those with a TTTF of more than 6 months, for example 7, 8, 9, 10, 11, 12, 13, 14, 15, 16, 17, 18, 19, 20, 21, 22, 23, 24 or more months.

The gene lists may be generated using Baldi analysis a variation of the classical T test, and/or Pearsons Correlation Coefficient and/or Linear Discriminant analysis. See for example Van't Veer L J, Dai H, van de Vijver M J, He Y D, Hart A A, Mao M, Peterse H L, van der Kooy K, Marton M J, Witteveen A T, et al. (2002) Gene expression profiling predicts clinical outcome of breast cancer. Nature, 415(6871), 530-556.

In one aspect the invention provides a gene profile generated by performing a pre-processing step to produce a gene intensity matrix and subjecting this matrix to a signal to noise statistical analysis to identify the differentially expressed genes and then ranking the genes in order of most differentially expressed gene.

In one method a threshold may be established by plotting a measure of the expression of the relevant gene for each patient. Generally the responders and the non-responders will be clustered about a different axis/focal point. A threshold can be established in the gap between the clusters by classical statistical methods or simply plotting a “best fit line” to establish the middle ground between the two groups. Values, for example, above the pre-defined threshold can be designated as responders and values, for example below the pre-designated threshold can be designated as non-responders.

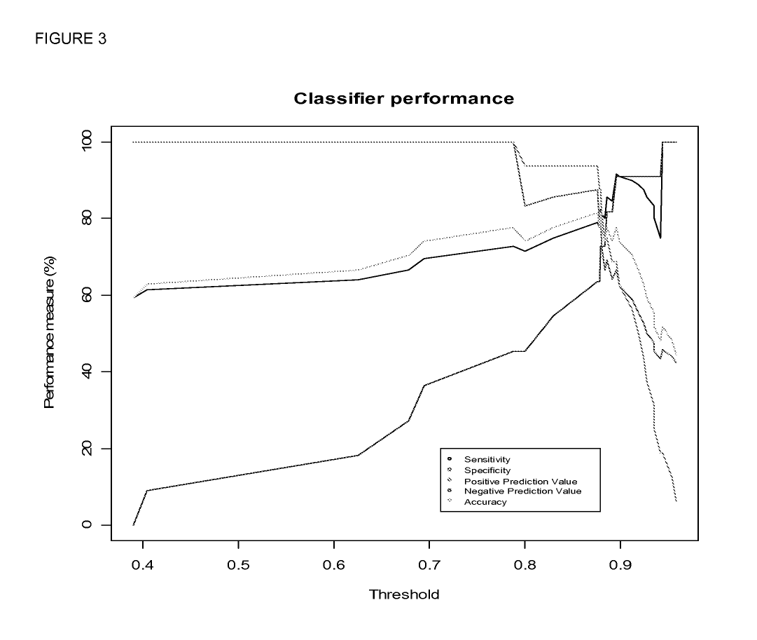

In one method the performance of any given classifier can be analysed. Exhaustive performance analysis is done by varying the level of the threshold and calculating, for each value of the threshold, the predictive ability of the model (sensitivity, specificity, positive and negative prediction value, accuracy). The results of one such an analysis are shown graphically in FIG. 3. This analysis can assist in selecting an appropriate threshold for a given classifier.

Based on the analysis in FIG. 3 setting the threshold at 0.9 will result in the best performance (best compromise) for that particular classifier. At this value of the threshold setting, the model has the following predictive performance on an independent set of samples:

### Sensitivity: 82%

### Specificity: 80%

### PPV: 87%

### NPV: 72%

### Accuracy: 81

The performance must be calculated for each specific classifier that may be employed. FIG. 3 and the information above is provided by way of example only.

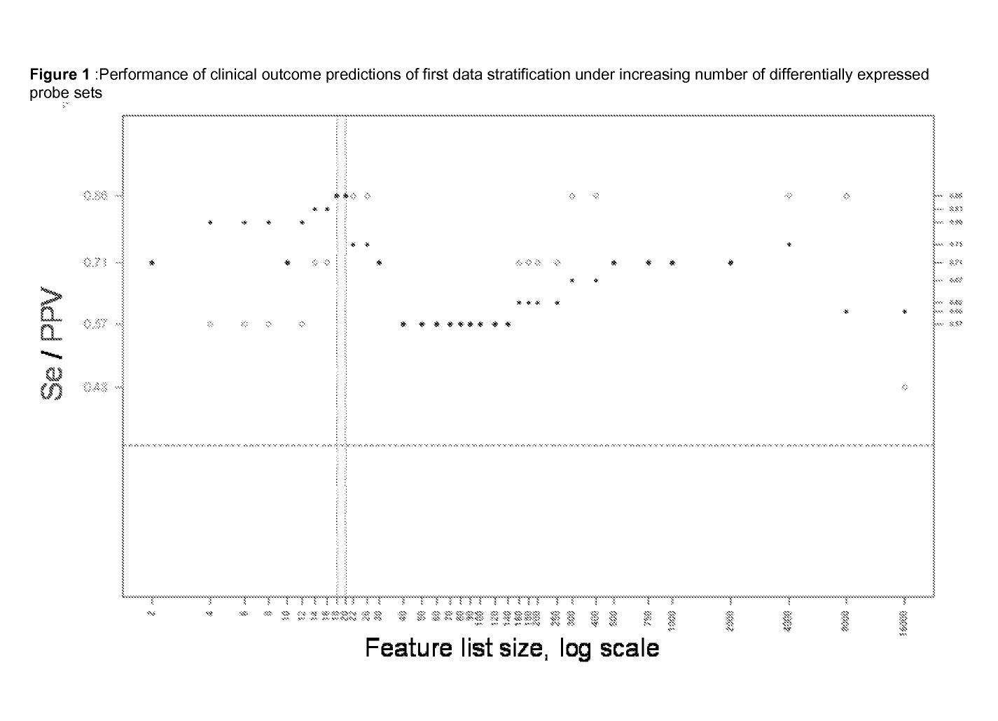

In addition performance analysis of the classifier can be done to for a given threshold value to evaluate the sensitivity, specificity, positive and negative prediction values and accuracy of the model. See for example FIG. 1 which shows a diagrammatic representation of the performance of the classifiers for a given threshold.

In one aspect the threshold is set at 0.5, 0.6, 0.7, 0.75, 0.8, 0.85 or 0.9.

In profiles provided by one or more aspects of the invention, the effect of genes that are closely correlated with gender are excluded. This may have an advantage that in these profiles are useful regardless of gender.

Once the gene profile has been identified and the analysis on the samples has been performed then there are a number of ways of presenting the results, for example as a heat map showing responders in one colour and non-responders in another colour. Nevertheless more qualitative information can be represented as an index that shows the results as a spectrum with a threshold, for example above the threshold patients are considered responders and below the threshold patients are considered to be non-responders. The advantage of presenting the information as a spectrum is that it allows a physician to decide whether to provide treatment for those patients thought to be non-responders, but who are located near the threshold.

“Immunotherapy” in the context of the invention means therapy based on stimulating an immune response, generally to an antigen, wherein the response results in the treatment, amelioration and/or retardation of the progression of a disease associated therewith. Treatment in this context would not usually include prophylactic treatment.

Cancer immunotherapy in the context of this specification means immunotherapy for the treatment of cancer. In one aspect the immunotherapy is based on a cancer testis antigen, such as MAGE (discussed in more detail below).

Advantageously, the novel method of the invention allows the identification of patients likely to respond to appropriate immunotherapy treatment. This facilitates the appropriate channelling of resources to patients who will benefit from them and what is more allow patients who will not benefit from the treatment to use alternative treatments that may be more beneficial for them.

This invention may be used for identifying cancer patients that are likely to respond to appropriate immunotherapy, for example patients with melanoma, breast, bladder, lung, NSCLC, head and neck cancer, squamous cell carcinoma, colon carcinoma and oesophageal carcinoma, such as in patients with MAGE-expressing cancers. In an embodiment, the invention may be used in an adjuvant (post-operative, for example disease-free) setting in such cancers, particularly lung and melanoma. The invention also finds utility in the treatment of cancers in the metastatic setting.

Thus in a first aspect the invention provides a signature indicative of a patient, such as a cancer patient, designated a responder or non-responder to treatment with an appropriate therapy, for example immunotherapy, the signature comprising differential expression of one or more genes selected from a gene list comprising or consisting a list of Table 1, 2, 4, 5, 6 or a mixture thereof.

“Immune activation gene” is intended to mean a gene whose product (eg mRNA or protein expressed from the gene) facilitates, increases, stimulates or is associated with an appropriate immune response. “Immune response gene” and “immune activation gene” are used interchangeably herein.

### Measurement of a Gene Profile/Level of Differential Expression

In the context of the present invention, the term “gene product” is intended to mean the mRNA or protein encoded by a gene, or cDNA that corresponds to the encoded mRNA.

An important technique for the analysis of the genes expressed by cells, such as cancer/tumour cells, is DNA microarray (also known as gene chip technology), where hundreds or more probe sequences (such as 55,000 probe sets) are attached to a glass surface. The probe sequences are generally all 25 mers or 60 mers and are sequences from known genes. These probes are generally arranged in a set of 11 individual probes (a probe set) and are fixed in a predefined pattern on the glass surface. Once exposed to an appropriate biological sample these probes hybridise to the relevant RNA or DNA of a particular gene. After washing, the chip is “read” by an appropriate method and a quantity such as colour intensity recorded. The differential expression of a particular gene is proportional to the measure/intensity recorded. This technology is discussed in more detail below.

Another useful technique for the measurement of protein gene products is through use of proteomic technology.

Once a target gene/profile has been identified there are several analytical methods to measure whether the gene(s)/profile(s) is/are differentially expressed. For DNA, these analytical techniques include real-time polymerase chain reaction, also called quantitative real time polymerase chain reaction (QRT-PCR or Q-PCR), which is used to simultaneously quantify and amplify a specific part of a given DNA molecule present in the sample.

The procedure follows the general pattern of polymerase chain reaction, but the DNA is quantified after each round of amplification (the “real-time” aspect). Two common methods of quantification are the use of fluorescent dyes that intercalate with double-strand DNA, and modified DNA oligonucleotide probes that fluoresce when hybridized with a complementary DNA.

The basic idea behind real-time polymerase chain reaction is that the more abundant a particular cDNA (and thus mRNA) is in a sample, the earlier it will be detected during repeated cycles of amplification. Various systems exist which allow the amplification of DNA to be followed and they often involve the use of a fluorescent dye which is incorporated into newly synthesised DNA molecules during real-time amplification. Real-time polymerase chain reaction machines, which control the thermocycling process, can then detect the abundance of fluorescent DNA and thus the amplification progress of a given sample. Typically, amplification of a given cDNA over time follows a curve, with an initial flat-phase, followed by an exponential phase. Finally, as the experiment reagents are used up, DNA synthesis slows and the exponential curve flattens into a plateau.

Alternatively the mRNA or protein product of the target gene(s) may be measured by Northern Blot analysis, Western Blot and/or immunohistochemistry.

In one aspect the methods or analyses described herein are performed on tumour samples in which a tumour associated antigen, for example a cancer testis antigen, is expressed.

When a single gene is analysed by, for example, Q-PCR then the gene expression can be normalised by reference to a gene that remains constant, for example genes with the symbol H3F3A, GAPDH, TFRC, GUSB or PGK1. The normalisation can be performed by subtracting the value obtained for the constant gene from the value obtained for the gene under consideration.

A threshold may be established by plotting a measure of the expression of the relevant gene for each patient. Generally the eg. responders and non-responders will be clustered about a different axis/focal point. A threshold can be established in the gap between the clusters by classical statistical methods or simply plotting a “best fit line” to establish the middle ground between the two groups. Values, for example, above the pre-defined threshold can be designated as responders and values, for example below the pre-designated threshold can be designated as non-responders.

### Microarrays

A microarray is an array of discrete regions, typically nucleic acids, which are separate from one another and are typically arrayed at a density of between, about 100/cm² to 1000/cm², but can be arrayed at greater densities such as 10000/cm². The principle of a microarray experiment, is that mRNA from a given cell line or tissue is used to generate a labelled sample typically labelled cDNA, termed the ‘target’, which is hybridized in parallel to a large number of, nucleic acid sequences, typically DNA sequences, immobilised on a solid surface in an ordered array.

Tens of thousands of transcript species can be detected and quantified simultaneously. Although many different microarray systems have been developed the most commonly used systems today can be divided into two groups, according to the arrayed material: complementary DNA (cDNA) and oligonucleotide microarrays. The arrayed material has generally been termed the probe since it is equivalent to the probe used in a northern blot analysis. Probes for cDNA arrays are usually products of the polymerase chain reaction (PCR) generated from cDNA libraries or clone collections, using either vector-specific or gene-specific primers, and are printed onto glass slides or nylon membranes as spots at defined locations. Spots are typically 10-300 μm in size and are spaced about the same distance apart. Using this technique, arrays consisting of more than 30,000 cDNAs can be fitted onto the surface of a conventional microscope slide. For oligonucleotide arrays, short 20-25 mers are synthesized in situ, either by photolithography onto silicon wafers (high-density-oligonucleotide arrays from Affymetrix or by ink-jet technology (developed by Rosetta Inpharmatics, and licensed to Agilent Technologies). Alternatively, presynthesised oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of pre-synthesised longer oligonucleotides (50-100 mers) has recently been developed to counteract these disadvantages.

Thus in performing a microarray to ascertain whether a patient presents a gene signature of the present invention, the following steps are performed: obtain mRNA from the sample and prepare nucleic acids targets, contact the array under conditions, typically as suggested by the manufactures of the microarray (suitably stringent hybridisation conditions such as 3×SSC, 0.1% SDS, at 50° C.) to bind corresponding probes on the array, wash if necessary to remove unbound nucleic acid targets and analyse the results.

It will be appreciated that the mRNA may be enriched for sequences of interest such as those in Table 1, 2, 4, 5 or 6 (or other embodiment of the invention) by methods known in the art, such as primer specific cDNA synthesis. The population may be further amplified, for example, by using PCR technology. The targets or probes are labelled to permit detection of the hybridisation of the target molecule to the microarray. Suitable labels include isotopic or fluorescent labels which can be incorporated into the probe.

Once a target gene/profile has been identified there are several alternative analytical methods to microarray that can be used to measure whether the gene(s) is/are differentially expressed.

Analytical techniques include real-time polymerase chain reaction, also called quantitative real time polymerase chain reaction (QRT-PCR or Q-PCR), which is used to simultaneously quantify and amplify a specific part of a given DNA molecule present in the sample.

PCR can be a more sensitive technique than microarray and may detect lower levels of differentially expressed genes. In one aspect of the present invention, the methods described herein, may use a PCR-based technique.

In one embodiment, a patient may be diagnosed to ascertain whether his/her tumour expresses the gene signature of the invention using a diagnostic kit based on PCR technology, in particular quantitative PCR (Q-PCR).

The procedure follows the general pattern of polymerase chain reaction, but the DNA is quantified after each round of amplification (the “real-time” aspect). Two common methods of quantification are the use of fluorescent dyes that intercalate with double-strand DNA, and modified DNA oligonucleotide probes that fluoresce when hybridized with a complementary DNA.

The basic idea behind real-time polymerase chain reaction is that the more abundant a particular cDNA (and thus mRNA) is in a sample, the earlier it will be detected during repeated cycles of amplification. Various systems exist which allow the amplification of DNA to be followed and they often involve the use of a fluorescent dye which is incorporated into newly synthesised DNA molecules during real-time amplification. Real-time polymerase chain reaction machines, which control the thermocycling process, can then detect the abundance of fluorescent DNA and thus the amplification progress of a given sample. Typically, amplification of a given cDNA over time follows a curve, with an initial flat-phase, followed by an exponential phase. Finally, as the experiment reagents are used up, DNA synthesis slows and the exponential curve flattens into a plateau.

Alternatively the mRNA or protein product of the target gene(s) may be measured by Northern Blot analysis, Western Blot and/or immunohistochemistry.

In one aspect the analysis to identify the profile/signature is performed on a patient sample wherein a cancer testis antigen is expressed.

When a single gene is analysed, for example, by Q-PCR then the gene expression can be normalised by reference to a gene that remains constant, for example genes with the symbol H3F3A, GAPDH, TFRC, GUSB or PGK1 may be suitable for employing in normalisation. The normalisation can be performed by subtracting the value obtained for the constant gene from the Ct value obtained for the gene under consideration.

One parameter used in quantifying the differential expression of genes is the fold change, which is a metric for comparing a gene's mRNA-expression level between two distinct experimental conditions. Its arithmetic definition differs between investigators. However, the higher the fold change the more likely that the differential expression of the relevant genes will be adequately separated, rendering it easier to decide which category (responder or non-responder) the patient falls into.

The fold change may, for example be at least 10, at least 15, at least 20 or 30.

Another parameter also used to quantify differential expression is the “p” value. It is thought that the lower the p value the more differentially expressed the gene is likely to be, which renders it a good candidate for use in profiles of the invention. P values may for example include 0.1 or less, such as 0.05 or less, in particular 0.01 or less. P values as used herein include corrected “P” values and/or also uncorrected “P” values.

This is thought to be in contrast to some other approaches which use genes that are strongly correlated with the disease/prognosis but not necessarily strongly differentially expressed.

As used herein, methods to predict a favourable clinical response or to identify subjects more likely to respond to therapy, is not meant to imply a 100% predictive ability, but to indicate that subjects with certain characteristics are more likely to experience a favourable clinical response to a specified therapy than subjects who lack such characteristics. However, as will be apparent to one skilled in the art, some individuals identified as more likely to experience a favourable clinical response may nonetheless fail to demonstrate measurable clinical response to the treatment. Similarly, some individuals predicted as non-responders may nonetheless exhibit a favourable clinical response to the treatment.

As used herein, a ‘favourable response’ (or ‘favourable clinical response’) to, for example, an anticancer treatment refers to a biological or physical response that is recognized by those skilled in the art as indicating a decreased rate of tumour growth, compared to tumour growth that would occur with an alternate treatment or the absence of any treatment. “Favourable clinical response” as used herein is not synonymous with a cure, but includes a “Response”, “Partial Response”, “Mixed Response” or “Stable Disease” as defined herein. A favourable clinical response to therapy may include a lessening of symptoms experienced by the subject, an increase in the expected or achieved survival time, a decreased rate of tumour growth, cessation of tumour growth (stable disease), regression in the number or mass of metastatic lesions, and/or regression of the overall tumour mass (each as compared to that which would occur in the absence of therapy, or in response to an alternate therapy).

Patients in need of treatment, for example, for cancer treatment such as for a MAGE-expressing tumour, and whose tumour cells have a gene signature described herein as a “Responder” signature are more likely to have a favourable clinical response, compared to patients whose tumour cells show a gene signature described herein as a “Non-Responder” signature, when treated with MAGE specific immunotherapy.

“Non-responder” in the context of this invention includes persons whose symptoms ie cancers/tumours are not improved or stabilised.

In one aspect of the invention “responder” may not include a “Mixed Responder”.

In one aspect the invention is greater than 50%, 60, 70 or 80% accurate such as about 81% accurate at predicting responders and non-responders correctly.

The invention also extends to separate embodiments according to the invention described herein, which comprise, consist essentially of, or consists of the components/elements described herein.

The invention extends to the functional equivalents of genes listed herein, for example as characterised by hierarchical classification of genes such as described by Hongwei Wu et al 2007(Hierarchical classification of equivalent genes in prokaryotes-Nucleic Acid Research Advance Access).

Whilst not wishing to be bound by theory, it is thought that is not necessarily the gene per se that is characteristic of the signature but rather it is the gene function which is fundamentally important. Thus a functionally equivalent gene to an immune activation gene such as those listed above, for example in Table 1, 2, 4, 5, 6 or mixtures thereof may be employed in the signature, see for example, Journal of the National Cancer Institute Vol 98, No. 7 Apr. 5, 2006.

The genes were identified by specific probes and thus a skilled person will understand that the description of the genes above is a description based on current understanding of what hybridises to the probe. However, regardless of the nomenclature used for the genes by repeating the hybridisation to the relevant probe under the prescribed conditions the requisite gene can be identified.

The invention extends to use of the profile(s) according to the invention for predicting or identifying a patient as a responder or non-responder to immunotherapy, such as cancer immunotherapy, for example cancer testis immunotherapy, in particular MAGE immunotherapy, especially for melanoma.

Thus the invention includes a method of analysing a patient derived sample, based on differential expression of the profile/gene(s) according to the invention for the purpose of characterising the patient from which the sample was derived as a responder or non-responder to immunotherapy according to the present invention

In one aspect the invention provides a method for measuring expression levels of polynucleotides from genes identified herein, in a sample for the purpose of identifying if the patient, from whom the sample was derived, is likely to be a responder or non-responder to immunotherapy such a cancer immunotherapy according to the present invention comprising the steps:
isolating the RNA from the sample,
optionally amplifying the copies of the cDNA from the sample for said genes, and
quantifying the levels of cDNA in the sample.

The invention provides a diagnostic kit comprising at least one component for performing an analysis on a patient derived sample to identify a profile according to the invention, the results of which may be used to designate a patient from which the sample was derived as a responder or non-responder to immunotherapy.

The kit may comprise materials/reagents for PCR (such as QPCR), microarray analysis, immunohistochemistry or other analytical technique that may be used for accessing differential expression of one or more genes.

The invention also provides a diagnostic kit comprising a set of probes capable of hybridising to the mRNA or cDNA of one or more, such as at least 5 genes described herein in relation to the invention, for example a diagnostic kit comprising or consisting of a set of probes capable of hybridising to the mRNA or its cDNA of 1, 2, 3, 4, 5, 6, 7, 8, 9, 10, 11, 12, 13, 14, 15, 16, 17 or all of the gene products recognised by the probe sets listed in Table 1.

The invention also provides a diagnostic kit comprising a set of probes capable of hybridising to the mRNA or cDNA of one or more, such as at least 5 genes described herein in relation to the invention, for example a diagnostic kit comprising or consisting of a set of probes capable of hybridising to the cDNA gene target sequence of 1, 2, 3, 4, 5, 6, 7, 8, 9, 10, 11, 12, 13, 14, 15, 16, 17 or all of the probe set target sequences listed in Table 9.

The invention also provides a diagnostic kit comprising or consisting of a set of probes capable of hybridising to the mRNA or its cDNA of one or more of 1-5,6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-5, 106-10, 111-5, 116-20, 121-5, 126-30, 131-5, 136-40, 141-5, 146-50, 151-5, 156-60, 161-65, 166-70, 171-5, 176-80, 181-5, 186-90, 191-5, 196-200, 201-5, 206-10, 211-5, 216-20, 221-5, 226-30, 231-5, 236-40, 241-5, 246-50 of the gene products recognised by the probe sets listed in Table 2 and/or any combination thereof.

The invention also provides a diagnostic kit comprising a set of probes capable of hybridising to the mRNA or cDNA of one or more, such as at least 5 genes described herein in relation to the invention, for example a diagnostic kit comprising or consisting of a set of probes capable of hybridising to the cDNA gene target sequence of 1-5,6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-5, 106-10, 111-5, 116-20, 121-5, 126-30, 131-5, 136-40, 141-5, 146-50, 151-5, 156-60, 161-65, 166-70, 171-5, 176-80, 181-5, 186-90, 191-5, 196-200, 201-5, 206-10, 211-5, 216-20, 221-5, 226-30, 231-5, 236-40, 241-5, 246-50 of the gene target sequences listed in Table 10, and/or any combination thereof.

The invention also provides a diagnostic kit comprising or consisting of a set of probes capable of hybridising to the mRNA or its cDNA of at least 5, 10, 15, 20, 25, 30, 35, 40, 45, 50, 55, 60, 65, 70, 75, 80, 85, 90, 95 or 100 of the genes recognised by the probe sets listed in Table 4.

The invention also provides a diagnostic kit comprising a set of probes capable of hybridising to the mRNA or cDNA of one or more, such as at least 5 genes described herein in relation to the invention, for example a diagnostic kit comprising or consisting of a set of probes capable of hybridising to the cDNA gene target sequence of at least 5, 10, 15, 20, 25, 30, 35, 40, 45, 50, 55, 60, 65, 70, 75, 80, 85, 90, 95 or 100 of the gene target sequences listed in Table 11.

The invention also provides a diagnostic kit comprising or consisting of a set of probes capable of hybridising to the mRNA or its cDNA of at least 5, 10, 15, 20, 25, 30, 35, 40, 45, 50, 55, 60, 65, 70, 75, 80, 85, 90, 95, 100 or 105 of the gene products recognised by the probe sets listed in Table 5.

The invention also provides a diagnostic kit comprising a set of probes capable of hybridising to the mRNA or cDNA of one or more, such as at least 5 genes described herein in relation to the invention, for example a diagnostic kit comprising or consisting of a set of probes capable of hybridising to the cDNA gene target sequence of at least 5, 10, 15, 20, 25, 30, 35, 40, 45, 50, 55, 60, 65, 70, 75, 80, 85, 90, 95, 100 or 105 of the gene target sequences listed in Table 12.

The invention also provides a diagnostic kit comprising or consisting of a set of probes capable of hybridising to the mRNA or cDNA of one or more, such as at least 5 genes described herein in relation to the invention, for example a diagnostic kit comprising or consisting of a set of probes capable of hybridising to the mRNA or its cDNA of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or all of the gene products recognised by the probe sets listed in Table 6.

The invention also provides a diagnostic kit comprising a set of probes capable of hybridising to the mRNA or cDNA of one or more, such as at least 5 genes described herein in relation to the invention, for example a diagnostic kit comprising or consisting of a set of probes capable of hybridising to the cDNA gene target sequence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or all of the gene target sequences listed in Table 13.

The methods may further comprise the step of determining whether the gene products described herein, in a patient-derived sample, are upregulated and/or downregulated, or the step of analysis of expression by Pearson, Baldi Correlation or Cox analysis, in order to determine whether the patient is a responder or non-responder to therapy.

In another embodiment this invention relates to diagnostic kits. For example, diagnostic kits containing such microarrays comprising a microarray substrate and probes that are capable of hybridising to mRNA or cDNA expressed from, for example, 5, 6, 7, 8, 9, 10, 11, 12, 13, 14, 15, 16, 17, 18, 19, 20 21, 22, 23, 24, 25, 26, 27, 28, 29, 30, 31 or more genes, for example from a particular table such as substantially all of the genes in Table 1, 2, 4, 5 or 6 that are capable of demonstrating the gene signature of the invention.

In one aspect the invention provides microarrays adapted for identification of a signature according to the invention.

The invention also extends to substrates and probes suitable for hybridising to an mRNA or cDNA moiety expressed from one or more genes employed in the invention, for example from Table 1, 2, 4, 5 or 6.

Commercially available microarrays contain many more probes than are required to characterise the differential expression of the genes under consideration at any one time, to aid the accuracy of the analysis. Thus one or more probe sets may recognise the same gene.

Thus in one embodiment multiple probes or probe sets are used to identify differential expression, such as upregulation, of a gene according to any aspect of the invention herein described.

The diagnostic kit may, for example, comprise probes, which are arrayed in a microarray.

Specifically, prepared microarrays, for example, containing one or more probe sets described herein can readily be prepared by companies such as Affymetrix, thereby providing a specific test and optionally reagents for identifying the profile, according to the invention.

In an embodiment the microarrays or diagnostic kits will additionally be able to test for the presence or absence of tumour associated antigen gene product, for example a cancer testis antigen expressing gene such as the MAGE gene, or for example the gene product of one or more of the following antigens: Her-2/neu; P501S; WT-1; a MAGE antigen, for example MAGE 1, MAGE 2, MAGE 3, MAGE 4, MAGE 5, MAGE 6, MAGE 7, MAGE 8, MAGE 9, MAGE 10, MAGE 11, MAGE 12, MAGE-B1, MAGE-B2, MAGE-B3 and MAGE-B4, MAGE-C1 and MAGE-C2; PRAME; LAGE 1; NY-ESO-1; SSX-2; SSX-4; SSX-5; NA17; MELAN-A; Tyrosinase; P790; P510; P835; B305D; B854; CASB618; CASB7439 (HASH-2); C1491; C1584; and C1585.

Thus in one aspect the invention provides a probe and/or probe set suitable for said hybridisation, under appropriate conditions. The invention also extends to use of probes, for example as described herein or functional equivalents thereof, for the identification of a gene profile according to the present invention.

The invention herein described extends to use of all permutations of the probes listed herein (or functional analogues thereof) for identification of the said signature.

In one aspect the invention provides use of a probe for the identification of differential expression of at least one gene product of an immune activation gene for establishing if a gene profile according to the present invention is present in a patient derived sample.

In one aspect, this invention relates to oligonucleotide probes and primers capable of recognising the gene products of the genes of Table 1, 2, 4, 5, and/or 6 or any other profile as described herein and diagnostic kits based on these probes and primers.

Such kits may include probes or kits for the detection of a tumour associated antigen, for example one or more of the following antigens: Her-2/neu; P501S; WT-1; a MAGE antigen, for example MAGE 1, MAGE 2, MAGE 3, MAGE 4, MAGE 5, MAGE 6, MAGE 7, MAGE 8, MAGE 9, MAGE 10, MAGE 11, MAGE 12, MAGE-B1, MAGE-B2, MAGE-B3 and MAGE-B4, MAGE-C1 and MAGE-C2; PRAME; LAGE 1; NY-ESO-1; SSX-2; SSX-4; SSX-5; NA17; MELAN-A; Tyrosinase; P790; P510; P835; B305D; B854; CASB618; CASB7439 (HASH-2); C1491; C1584; and C1585.

In embodiments of the present invention in which hybridisation is employed, hybridisation will generally be preformed under stringent conditions, such as 3×SSC, 0.1% SDS, at 50° C.

Once target gene(s)/profile has/have been identified then it is well within the skilled person's ability to design alternative probes that hybridise to the same target. Therefore the invention also extends to probes, which under appropriate conditions measure the same differential expression of the gene(s) of the present invention to provide a signature/profile as described.

The invention also extends to use of the relevant probe in analysis of whether a cancer patient will be a responder or non-responder to treatment with an appropriate immunotherapy.

The invention also extends to use (and processes or methods employing same) of known microarrays for identification of a signature according to the invention.

A nucleic acid probe may be at least 10, 15, 20, 25, 30, 35, 40, 50, 75, 100 or more nucleotides in length and may comprise the full length gene. Probes for use in the invention are those that are able to hybridise specifically to the mRNA (or its cDNA) expressed from the genes listed in Table 1 2, 4, 5 or 6 under stringent conditions.

The invention further provides a method for the detection of a gene profile in a biological sample, the method comprising measuring the expression of 1, 2, 3, 4, 5, 6, 7, 8, 9, 10, 11, 12, 13, 14, 15, 16, 17 or all of the gene products recognised by the probe sets listed in Table 1. Alternatively, the method may comprise measuring the expression of the gene product recognised by 1-5,6-10, 11-18 of the probe sets listed in Table 1 and/or any combination thereof.

The invention further provides a method for the detection of a gene profile in a biological sample, the method comprising measuring the expression of one or more of 1-5,6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-5, 106-10, 111-5, 116-20, 121-5, 126-30, 131-5, 136-40, 141-5, 146-50, 151-5, 156-60, 161-65, 166-70, 171-5, 176-80, 181-5, 186-90, 191-5, 196-200, 201-5, 206-10, 211-5, 216-20, 221-5, 226-30, 231-5, 236-40, 241-5, 246-50 of the gene products recognised by the probe sets listed in Table 2, and/or any combination thereof.

The invention further provides a method for the detection of a gene profile in a biological sample, the method comprising measuring the expression of at least 5, 10, 15, 20, 25, 30, 35, 40, 45, 50, 55, 60, 65, 70, 75, 80, 85, 90, 95 or 100 of the gene products recognised by the probe sets listed in Table 4.

The invention further provides a method for the detection of a gene profile in a biological sample, the method comprising measuring the expression of at least 5, 10, 15, 20, 25, 30, 35, 40, 45, 50, 55, 60, 65, 70, 75, 80, 85, 90, 95, 100 or 105 of the gene products recognised by the probe sets listed in Table 5.

The invention further provides a method for the detection of a gene profile in a biological sample, the method comprising measuring the expression of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or all of the gene products recognised by the probe sets listed in Table 6.

In one embodiment of the present invention there is provided a method for classifying a patient as a responder or non-responder to therapy comprising measuring, in a patient-derived sample, the gene product of at least one gene selected from the genes listed in Table 1. The method may comprise measuring the gene product of 1, 2, 3, 4, 5, 6, 7, 8, 9, 10, 11, 12, 13, 14, 15, 16, 17 or 18 of the genes recognised by the probe sets listed in Table 1 and/or any combination thereof. Alternatively, the method may comprise measuring the gene product of at least 1, 5, 10, 15 or all of the genes of Table 1 or the genes recognised by the probe sets listed in Table 1. The methods may further comprise the step of using the data of Appendix A to determine whether a patient is a responder or non-responder.

In one embodiment of the present invention there is provided a method for classifying a patient as a responder or non-responder to therapy comprising measuring, in a patient-derived sample, the gene product of at least one gene selected from the genes listed in Table 2. The method may comprise measuring the gene product of one or more of 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-5, 106-10, 111-5, 116-20, 121-5, 126-30, 131-5, 136-40, 141-5, 146-50, 151-5, 156-60, 161-65, 166-70, 171-5, 176-80, 181-5, 186-90, 191-5, 196-200, 201-5, 206-10, 211-5, 216-20, 221-5, 226-30, 231-5, 236-40, 241-5, 246-50 of the gene products recognised by the probe sets listed in Table 2 and/or any combination thereof. The methods may further comprise the step of using the data of Appendix B to determine whether a patient is a responder or non-responder.

In one embodiment of the present invention there is provided a method for classifying a patient as a responder or non-responder to therapy comprising measuring, in a patient-derived sample, the gene product of at least one gene selected from the genes listed in Table 4. The method may comprise measuring the gene product of at least 5, 10, 15, 20, 25, 30, 35, 40, 45, 50, 55, 60, 65, 70, 75, 80, 85, 90, 95 or 100 of the genes recognised by the probe sets listed in Table 4. The methods may further comprise the step of classifying a sample using the value of the probe set ID in the metagene, provided in Table 7.

In one embodiment of the present invention there is provided a method for classifying a patient as a responder or non-responder to therapy comprising measuring, in a patient-derived sample, the gene product of at least one gene selected from the genes listed in Table 5. The method may comprise measuring the gene product of at least 5, 10, 15, 20, 25, 30, 35, 40, 45, 50, 55, 60, 65, 70, 75, 80, 85, 90, 95, 100 or 105 of the gene products recognised by the probe sets listed in Table 5. The methods may further comprise the step of classifying a sample using the value of the probe set ID in the metagene, provided in Table 8.

In one embodiment of the present invention there is provided a method for classifying a patient as a responder or non-responder to therapy comprising measuring, in a patient-derived sample, the gene product of at least one gene selected from the genes listed in Table 6. The method may comprise measuring the gene product of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or all of the gene products recognised by the probe sets listed in Table 6. The method may further comprise the step of using SPCA to analyse the level of expression of a gene product in a sample, for example by setting a threshold to classify responders and non-responders as described herein.

Thus in one aspect the invention provides a method of identifying whether a patient is a responder or non-responder to therapy, following surgical resection of a tumour, the method comprising: analysing a sample for differential expression of one or more genes or gene profiles as described herein and/or listed or shown in the Tables shown herein; and characterising a patient as being a responder or non-responder.

Thus in one aspect the invention provides a method of identifying whether a patient will be a responder or non-responder to immunotherapy, the method comprising: (a) analysing a patient-derived sample comprising mRNA or fragments thereof expressed by genes of cancerous cells or DNA or fragments thereof from cancerous cells, for differential expression of one or more genes indicative of T-cell infiltration/activation, for example selected from the group comprising or consisting of genes listed in Table 1, 2, 4, 5 or 6, and (b) characterising a patient as a responder or a non-responder based on the results of step (a).

In one embodiment of the invention described herein, the patient is a patient suffering from cancer or having a tumour, or a patient having had surgical removal or resection of a tumour or tumour tissue.

The present invention further relates to a method of screening the effects of a drug or therapy on a tissue or cell sample comprising the step of analysing the expression profile, employing any embodiment of the invention described herein before and after drug treatment or therapy. The invention therefore provides a method for screening for a drug or therapy, which would alter the gene profile to that of a patient having improved survival following treatment with, for example, MAGE antigen specific cancer immunotherapy (ie. to alter the gene profile to that of a responder), to enable the patient to benefit from, for example, MAGE antigen specific cancer immunotherapy.

In one embodiment, administration of a drug or therapy may be used to alter a patient's profile to that of a responder. The drug or therapy may comprise the drug or therapy identified through the method of screening described above. In one embodiment, the drug or therapy comprises topical administration of imiquimod: such topical administration is particularly suitable for a gene profile of external lesions or tumours, for example skin lesions. In one embodiment, the drug or therapy is local irradiation of the tumour. In one embodiment, the drug or therapy is selected from the group comprising: IL-2, IFN-α, dimethyltrizenoimidazolcarboxam (dacarbazine; DTIC) and temozolomide (TMZ).

In one embodiment, the table below describes possible drug or therapy administration that may be used to alter a profile:

| Tumor type | Tumor stage | Treatment |
|---|---|---|
| Metastatic | Unresectable | DTIC or TMZ as first-line treatment |
| melanoma | stage III or IV | First-line chemotherapy treatment other than DTIC only or |
| | | TMZ only (that may, but need not, include DTIC, TMZ, IL-2 or |
| | | IFNα) |
| | | Any second-line chemotherapy treatment |
| | | Local irradiation of cutaneous/subcutaneous tumor lesions |
| | | Local imiquimod |
| Non-small- | Any stage, if the | Chemo(radio)-therapy induction doublet neo-adjuvant |
| cell lung | patient is eligible | chemotherapy with platinum plus a second chemotherapy |
| cancer | for neo-adjuvant | drug. |
| | chemotherapy | [Note: Thus, induction radiotherapy is permitted.] |
| | with subsequent | |
| | resection | |

DTIC, dimethyltrizenoimidazolcarboxam (dacarbazine);
TMZ, temozolomide.

The present invention further provides a method of patient diagnosis comprising, for example, the step of analysing the expression profile according to any embodiment of the invention described herein and comparing it with a pre-determined standard to determine whether a patient is a “responder” and benefit from therapy, for example MAGE specific immunotherapy.

The invention includes a method of patient diagnosis comprising the step of analysing the expression profile according to any embodiment of the invention from a tumour tissue sample given by a patient and assessing, for example whether 1, 2, 3, 4, 5 or more of said genes are expressed, or comprising the step of using any method described herein.

Thus in clinical applications, tissue samples from a human patient may be screened for the presence and/or absence of the expression of, any embodiment of the invention described herein.

In an alternative aspect the invention provides a method further comprising the steps of analysing a tumour derived sample to determine which antigen(s) are expressed by the tumour and hence enabling administration of an a therapeutically effective amount of an appropriate therapy, for example an antigen specific cancer immunotherapeutic, for example where the tumour is found to be MAGE (such as MAGE A3) positive, appropriate treatment may, for example, include administration of MAGE A3 antigen specific immunotherapy.

### Samples

A sample such as tumour tissue of a patient is deemed to present the gene signature of the invention if one or more genes, such as described in any embodiment of the invention are differentially expressed (such as upregulated), and can be detected by microarray analysis or other appropriate analysis for example as described herein.

Thus in clinical applications, tissue samples from a human patient may be screened for the presence and/or absence of differential expression of a gene profile as described herein.

In the context of the present invention, the sample may be of any biological tissue or fluid derived from a patient potentially in need of treatment. The sample maybe derived from sputum, blood, urine, or from solid tissues such as biopsy from a primary tumour or metastasis, or from sections of previously removed tissues.

Samples could comprise or consist of, for example, needle biopsy cores, surgical resection samples or lymph node tissue. These methods include obtaining a biopsy, which is optionally fractionated by cryostat sectioning to enrich tumour cells to about 80% of the total cell population. In certain embodiments, nucleic acids extracted from these samples may be amplified using techniques well known in the art. The levels of selected markers in a profile can be detected and can be compared with statistically valid groups of, for example, MAGE positive non responder patients.

For cancer, the biological sample may contain cancer or tumour cells and may, for example, be derived from the cancer or tumour such as a fresh sample (including frozen samples) or a sample that has been preserved in paraffin. Having said this, samples preserved in paraffin can suffer from degradation and a profile observed may be modified. A person working the in field is well able to compensate of these changes observed by recalibrating the parameters of the profile.

In one aspect the biological sample is a biopsy sample, for example from a tumour or cancerous tissue.

Tables with Gene Lists

In one aspect a gene profile of the present invention comprises one or more genes listed in Table 1; in another aspect the profile comprises one or more genes listed in Table 2; in another aspect the profile comprises one or more genes listed in Table 4; in another aspect the profile comprises one or more genes listed in Table 5; in another aspect the profile comprises one or more genes listed in Table 6.

The tables list probe sets specific for certain regions of genes. In the tables listed herein a given gene may be listed more than once, for example as a specific gene or as a gene cluster. The given gene may be listed more than once because more than one probe set is listed in the table that is specific for the given gene.

In one aspect a profile comprises one or more genes selected from the genes or probe sets of Tables 1, 2, 4, 5 and/or 6 or one or more genes recognised by probe sets selected from the genes or probe sets of Tables 1, 2, 4, 5 and/or 6 including combinations thereof.

In a further aspect a profile comprises all the genes listed in Table 1 or genes recognised by probe sets listed in Table 1, all the genes listed in Table 2 or genes recognised by probe sets listed in Table 2, all the genes listed in Table 4 or genes recognised by probe sets listed in Table 4, all the genes listed in Table 5 or genes recognised by probe sets listed in Table 5 and/or all the genes listed in Table 6 or genes recognised by probe sets listed in Table 6 or combinations thereof.

In a further aspect a profile comprises at least 10% of the genes listed in Tables 1, 2, 4, 5 and/or 6 or genes recognised by the probe sets listed in the Tables, for example at least 40%, 50%, 60% or 70% such as 80%, 90% or 100% thereof.

One or more genes of Table 1 or genes recognises by probe sets of Table 1, as described herein, may include 1, 2, 3, 4, 5, 6, 7, 8, 9, 10, 11, 12, 13, 14, 15, 16, 17 or all of the genes or probe sets of Table 1, as appropriate, and/or any combination thereof.

One or more genes of Table 2 or genes recognises by probe sets of Table 2, as described herein, may include 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-5, 106-10, 111-5, 116-20, 121-5, 126-30, 131-5, 136-40, 141-5, 146-50, 151-5, 156-60, 161-65, 166-70, 171-5, 176-80, 181-5, 186-90, 191-5, 196-200, 201-5, 206-10, 211-5, 216-20, 221-5, 226-30, 231-5, 236-40, 241-5, 246-50 of the probe sets or genes of Table 2, as appropriate, and/or any combination thereof.

One or more genes of Table 4 or genes recognises by probe sets of Table 4, as described herein, may include 5, 10, 15, 20, 25, 30, 35, 40, 45, 50, 55, 60, 65, 70, 75, 80, 85, 90, 95 or 100 of the probe sets or genes of Table 4, as appropriate, and/or any combination thereof.

One or more genes of Table 5 or genes recognises by probe sets of Table 5, as described herein, may include 5, 10, 15, 20, 25, 30, 35, 40, 45, 50, 55, 60, 65, 70, 75, 80, 85, 90, 95, 100 or 105 of the probe sets or genes of Table 5, as appropriate, and/or any combination thereof.

One or more genes of Table 6 or genes recognises by probe sets of Table 6, as described herein, may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or all of the probe sets or genes of Table 6, as appropriate, and/or any combination thereof.

### Immunotherapeutic Agents, or Immunotherapy

The terms “immunotherapeutics”, “immunotherapeutic agents” and “immunotherapy” are used interchangeably herein. The terms “MAGE specific immunotherapy” and “MAGE antigen specific immunotherapy” and other similar terms are used interchangeably herein.

In one embodiment, the therapy or immunotherapy for use in the present invention comprises or consists of, for example, a composition or immunogenic composition comprising a tumour associated antigen as described herein. The tumour associated antigen may be a MAGE antigen or derivative as described herein.

The therapy as described herein may be used or administered to prevent or ameliorate recurrence of disease. Such treatment may be given after resection by surgery of any tumour or after chemotherapy or radiotherapy treatment.

A further aspect of the invention is a method of treating a patient suffering from a tumour, for example a MAGE antigen expressing tumour, the method comprising determining whether a patient's tumour expresses a gene profile as described herein and then administering a composition or an immunogenic composition comprising a tumour associated antigen as described herein, for example a MAGE antigen as described herein, or by administration of a MAGE antigen specific immunotherapy to said patient.

By “antigen specific immunotherapy”, “antigen specific cancer immunotherapy” and/or “cancer immunotherapy” is meant a composition that targets an antigen, through administration of a composition comprising an antigen (active immunotherapy) as described herein, or through administration of an antibody or other specific binding agent that targets the antigen (passive immunotherapy). For example, “MAGE antigen specific immunotherapy”, “MAGE immunotherapy” or “MAGE specific immunotherapy” may comprise or consist of administration of a composition comprising a MAGE antigen or derivative as described herein.

In one embodiment of the invention, the antigen to be targeted is MAGE. The MAGE antigen or derivative as described herein may comprise a full length MAGE antigen or a peptide thereof as described herein. In one embodiment the antigen or derivative may comprise a MAGE-A3 protein or peptide, a MAGE antigen comprising the peptide or epitopes EVDPIGHLY (SEQ ID NO:6094), FLWGPRALV (SEQ ID NO:6095); MEVDPIGHLY (SEQ ID NO:6096); VHFLLLKYRA (SEQ ID NO:6097); LVHFLLLKYR (SEQ ID NO:6098); LKYRAREPVT (SEQ ID NO:6099); ACYEFLWGPRALVETS (SEQ ID NO:6100); and TQHFVQENYLEY (SEQ ID NO:6101); and/or a MAGE antigen or peptide as described herein fused or conjugated to a carrier protein as described herein, for example in which the carrier protein is selected from protein D, NS1 or CLytA or fragments thereof.

The composition or immunogenic composition as described herein may further comprise an adjuvant as described herein For example, the adjuvant may comprise one or more or any combination of: 3D-MPL; aluminium salts; CpG containing oligonucleotides; saponin-containing adjuvants such as QS21 or ISCOMs; oil-in-water emulsions; and liposomes.

The invention further provides the use of composition or immunogenic composition as described herein comprising a tumour associated antigen as described herein in the manufacture of a medicament for the treatment of patients determined to have or characterised as having a poor prognosis according to methods described herein.

In one aspect the invention provides use of a composition or immunotherapy as described herein in the manufacture of a medicament for the treatment of patients suffering from or susceptible to recurrence of a tumour, for example a MAGE antigen expressing tumour, in which the patient expresses a gene profile or gene product(s) as described herein.

Antigen Specific Cancer Immunotherapy (ASCIs) suitable for use in the invention may, for example include those capable of raising an antigen-specific immune response, for example a MAGE specific immune response. In one embodiment, the ASCI comprises a MAGE antigen as described herein.

Alternatively, for treatment of tumours expressing other antigens, other compositions or immunogenic compositions or ASCIs may be used. Examples of compositions or ASCIs suitable for use in the present invention include compositions or ASCIs containing antigens or cancer testis antigens such as PRAME, LAGE 1, NY-ESO-1 (LAGE 2), and others, for example details of which can be obtained from www.cancerimmunity.org/CTdatabase.

The compositions, immunogenic compositions, ASCI's or cancer immunotherapy may be based, for example, on one or more of the antigens discussed herein, or derivatives of such antigens, for example as described herein.

### Antigens

In one embodiment of the present invention, the antigen may consist or comprise of a MAGE tumour antigen, for example, MAGE 1, MAGE 2, MAGE 3, MAGE 4, MAGE 5, MAGE 6, MAGE 7, MAGE 8, MAGE 9, MAGE 10, MAGE 11 or MAGE 12. The genes encoding these MAGE antigens are located on chromosome X and share with each other 64 to 85% homology in their coding sequence. These antigens are sometimes known as MAGE A1, MAGE A2, MAGE A3, MAGE A4, MAGE A5, MAGE A6, MAGE A7, MAGE A8, MAGE A9, MAGE A 10, MAGE A11 and/or MAGE A12 (the MAGE A family). In one embodiment, of the present invention, the antigen is MAGE-A3.

In one embodiment, an antigen from one of two further MAGE families may be used: the MAGE B and MAGE C group. The MAGE B family includes MAGE B1 (also known as MAGE Xp1, and DAM 10), MAGE B2 (also known as MAGE Xp2 and DAM 6) MAGE B3 and MAGE B4—the MAGE C family currently includes MAGE C1 and MAGE C2.

In general terms, a MAGE protein can be defined as containing a core sequence signature located towards the C-terminal end of the protein (for example with respect to MAGE A1 a 309 amino acid protein, the core signature corresponds to amino acid 195-279). In general, a MAGE protein may be approximately 50% or more identical, such as 70, 80, 90, 95 or 99% identical, in this core region with amino acids 195 to 279 of MAGE A1.

The consensus pattern of the core signature is thus described as follows wherein x represents any amino acid, lower case residues are conserved (conservative variants allowed) and upper case residues are perfectly conserved.

### Core Sequence Signature

LixvL(2x)I(3x)g(2x)apEExiWexl(2x)m(3-4x)Gxe(3-4x)gxp(2x)IIt(3x)VqexYLxYxqVPxsxP(2x)yeFLWGprA(2x)Et(3x)kv Conservative substitutions are well known and are generally set up as the default scoring matrices in sequence alignment computer programs. These programs include PAM250 (Dayhoft M. O. et al., (1978), “A model of evolutionary changes in proteins”, In “Atlas of Protein sequence and structure” 5(3) M. O. Dayhoft (ed.), 345-352), National Biomedical Research Foundation, Washington, and Blosum 62 (Steven Henikoft and Jorja G. Henikoft (1992), “Amino acid substitution matricies from protein blocks”), Proc. Natl. Acad. Sci. USA 89 (Biochemistry): 10915-10919.

In general terms, substitution within the following groups are conservative substitutions, but substitutions between groups are considered non-conserved. The groups are:
i) Aspartate/asparagine/glutamate/glutamine
ii) Serine/threonine
iii) Lysine/arginine
iv) Phenylalanine/tyrosine/tryptophane
v) Leucine/isoleucine/valine/methionine
vi) Glycine/alanine

In general and in the context of this invention, a MAGE protein will be approximately 50% or more identical, such as 70, 80, 90, 95 96, 97, 98 or 99% identical, in this core region with amino acids 195 to 279 of MAGE A1.

MAGE protein derivatives are also known in the art, see: WO 99/40188. Such derivatives are suitable for use in the present invention, for example in compositions, immunogenic compositions, therapeutic vaccine formulations or immunotherapy as described herein, which are suitable for the treatment of a range of tumour types.

Several CTL epitopes have been identified on the MAGE-3 protein and may comprise or consist of an antigen of the present invention. One such epitope, MAGE-3.A1, is a nonapeptide sequence located between amino acids 168 and 176 of the MAGE-3 protein which constitutes an epitope specific for CTLs when presented in association with the MHC class I molecule HLA.A1.

In alternative embodiments of the invention, the antigen may comprise or consist of one of the following antigens, or an immunogenic portion thereof which is able to direct an immune response to the antigen: SSX-2; SSX-4; SSX-5; NA17; MELAN-A; Tyrosinase; LAGE-1; NY-ESO-1; PRAME; P790; P510; P835; B305D; B854; CASB618 (as described in WO00/53748); CASB7439 (HASH-2, also described in WO01/62778); C1491; C1584; and C1585.

In one embodiment, the antigen may comprise or consist of P501S (also known as protein). The P501S antigen may be a recombinant protein that combines most of the P501S protein with a bacterial fusion protein comprising the C terminal part of protein LytA of Streptococcus pneumoniae in which the P2 universal T helper peptide of tetanus toxoid has been inserted, ie. a fusion comprising CLytA-P2-CLyta (the “CPC” fusion partner), as described in WO03/104272;

In one embodiment, the antigen may comprise or consist of WT-1 expressed by the Wilm's tumor gene, or its N-terminal fragment WT-1F comprising about or approximately amino acids 1-249; the antigen expressed by the Her-2/neu gene, or a fragment thereof.

In one embodiment, the Her-2/neu antigen may be one of the following fusion proteins which are described in WO00/44899.

In a further embodiment, the antigen may comprise or consist of “HER-2/neu ECD-ICD fusion protein,” also referred to as “ECD-ICD” or “ECD-ICD fusion protein,” which refers to a fusion protein (or fragments thereof) comprising the extracellular domain (or fragments thereof) and the intracellular domain (or fragments thereof) of the HER-2/neu protein. In one embodiment, this ECD-ICD fusion protein does not include a substantial portion of the HER-2/neu transmembrane domain, or does not include any of the HER-2/neu transmembrane domain.

In a further embodiment, the antigen may comprise or consist of “HER-2/neu ECD-PD fusion protein,” also referred to as “ECD-PD” or “ECD-PD fusion protein,” or the “HER-2/neu ECD-ΔPD fusion protein,” also referred to as “ECD-ΔPD” or “ECD-ΔPD fusion protein,” which refers to fusion proteins (or fragments thereof) comprising the extracellular domain (or fragments thereof) and phosphorylation domain (or fragments thereof, e.g., ΔPD) of the HER-2/neu protein. In one embodiment, the ECD-PD and ECD-ΔPD fusion proteins do not include a substantial portion of the HER-2/neu transmembrane domain, or does not include any of the HER-2/neu transmembrane domain.

In one embodiment, the antigen as described herein may be linked to an immunological fusion or expression enhancer partner. Fusion proteins may include a hybrid protein comprising two or more antigens relevant to a given disease or may be a hybrid of an antigen and an expression enhancer partner.

The antigen and partner may be chemically conjugated, or may be expressed as a recombinant fusion protein. In one embodiment the antigen and partner are expressed as a recombinant fusion protein. The fusion partner may assist in providing T helper epitopes (immunological fusion partner) and/or assist in expressing the protein at higher yields than the native recombinant protein (expression enhancer). In one embodiment, the fusion partner may be both an immunological fusion partner and expression enhancing partner.

In one embodiment of the invention, the immunological fusion partner that may be used is derived from protein D, a surface protein of the gram-negative bacterium, Haemophilus influenza B (WO 91/18926) or a derivative thereof. Thus, fusion partner proteins for use in the present invention may, for example, be derived from protein D. Protein D is a lipoprotein (a 42 kDa immunoglobulin D binding protein exposed on the surface of the Gram-negative bacterium Haemophilus influenzae). The protein is synthesized as a precursor with an 18 amino acid residue signal sequence, containing a consensus sequence for bacterial lipoprotein (WO 91/18926). Native precursor Protein D protein is processed during secretion and the signal sequence is cleaved. The Cys of the processed Protein D (at position 19 in the precursor molecule) becomes the N terminal residue of the processed protein and is concomitantly modified by covalent attachment of both ester-linked and amide-linked fatty acids. The fatty acids linked to the amino-terminal Cysteine residue then function as membrane anchor.

In one embodiment, the tumour associated antigen or derivative for use in the present invention may comprise Protein D or a derivative thereof as a fusion partner protein.

The protein D or a derivative thereof as described herein may comprise, for example: the first or N-terminal third of processed protein D or approximately or about the first or N-terminal third of processed protein D. In one embodiment, the protein D or a derivative thereof may comprise the first or N-terminal 100 to 115 amino acids of processed protein D; or the first or N-terminal 109 amino acids of processed protein D. In one embodiment, the native processed Protein D amino acids 2-Lys and 2-Leu may be substituted with amino acids 2-Asp and 3-Pro.

In one embodiment, it may comprise the first N-terminal 100-110 amino acids or approximately the first N-terminal 100-110 amino acids. In one embodiment the fusion protein comprises the first 109 residues (or 108 residues therefrom) or amino acids 20 to 127 of protein D.

In one embodiment, the protein D or derivative thereof may further include the 18 or 19 amino acid signal sequence of precursor protein D. In one embodiment, the fusion partner protein derived from protein D comprises or consists of amino acids 20 to 127 of precursor protein D. In one embodiment of the present invention, the two amino acids 21-Lys and 22-Leu of the precursor protein D fusion partner protein may be substituted with amino acids 21-Asp and 22-Pro.

The protein D fusion partner protein as described herein may additionally or alternatively contain deletions, substitutions or insertions within the amino acid sequence when compared to the wild-type precursor or processed protein D sequence. In one embodiment, 1, 2, 3, 4, 5, 6, 7, 8, 9 or more amino acids may be inserted, substituted or deleted. The amino acids may be substituted with conservative substitutions as defined herein, or other amino acids may be used.

In one embodiment, the fusion partner protein may comprise or consist of the protein shown in SEQ ID NO: 6092 (FIG. 12). In one embodiment, the fusion partner protein may comprise or consist of the amino acids underlined in FIG. 12.

Other fusion partners that may be used include the non-structural protein from influenzae virus, NS1 (hemagglutinin). Typically the N terminal 81 amino acids of NS1 may be utilised, although different fragments may be used provided they include T-helper epitopes.

In another embodiment the fusion partner protein for use in the present invention is LytA, for example C-LytA. LytA is derived from Streptococcus pneumoniae which synthesise an N-acetyl-L-alanine amidase, amidase LytA, (coded by the LytA gene (Gene, 43 (1986) page 265-272) an autolysin that specifically degrades certain bonds in the peptidoglycan backbone. The C-terminal domain of the LytA protein (C-LytA or CLytA) is responsible for the affinity to the choline or to some choline analogues such as DEAE. This property has been exploited for the development of E. coli C-LytA expressing plasmids useful for expression of fusion proteins. Purification of hybrid proteins containing the C-LytA fragment at its amino terminus has been described (Biotechnology: 10, (1992) page 795-798). In one embodiment, the C terminal portion of the molecule may be used. The embodiment may utilise the repeat portion of the LytA molecule found in the C terminal end starting at residue 178. In one embodiment, the LytA portion may incorporate residues 188-305.

In one embodiment of the present invention, the MAGE protein may comprise a derivatised free thiol. Such antigens have been described in WO 99/40188. In particular carboxyamidated or carboxymethylated derivatives may be used.

In one embodiment of the present invention, the tumour associated antigen comprises protein D-MAGE-A3 as a fusion protein. The nucleotide and amino acid sequences for this molecule are shown in FIG. 11 and SEQ ID NO:6093. This antigen is described in more detail in WO 99/40188.

In further embodiments of the present invention, the tumour associated antigen may comprise any of the following fusion proteins: a fusion protein of Lipoprotein D fragment, MAGE1 fragment, and histidine tail; fusion protein of NS1-MAGE3, and Histidine tail; fusion protein of CLYTA-MAGE1-Histidine; fusion protein of CLYTA-MAGE3-Histidine.

In further embodiments of the present invention, the tumour associated antigen may comprise a fusion protein as described herein.

In a further embodiment of the present invention, the composition or immunogenic composition may comprise a nucleic acid molecule encoding an antigen or derivative as described herein, for example a nucleic acid-based vaccine or immunogenic composition may be used. This may comprise a nucleic acid molecule encoding an antigen or fusion protein as described herein. Nucleic acid sequences may be administered directly, as part of particle-mediated delivery (PMED), and/or may be inserted into a suitable expression vector and used for DNA/RNA vaccination. Such sequences may be inserted into a suitable expression vector and used for DNA/RNA vaccination. Microbial vectors expressing the nucleic acid may also be used as vectored delivered immunotherapeutics. Such vectors include for example, poxvirus, adenovirus, alphavirus and listeria.

Conventional recombinant techniques for obtaining nucleic acid sequences, and production of expression vectors of are described in Maniatis et al., Molecular Cloning—A Laboratory Manual; Cold Spring Harbor, 1982-1989.

Compositions, vaccines, immunotherapeutic agents or components thereof as described herein for use in the present invention, for example protein components, are provided either in a liquid form or in a lyophilised form.

Each human dose may comprise 1 to 1000 μg of protein, for example 30-300 μg such as 25, 30, 40, 50, 60, 70, 80 or 90 μg.

The compositions, immunogenic compositions, vaccines or immunotherapeutic agents or methods described herein may further comprise an adjuvant, and/or an immunostimulatory cytokine or chemokine.

Adjuvants that may be used in the present invention include Merck Adjuvant 65 (Merck and Company, Inc., Rahway, N.J.); aluminium salts such as aluminium hydroxide gel (alum) or aluminium phosphate; salts of calcium, iron or zinc; an insoluble suspension of acylated tyrosine; acylated sugars; cationically or anionically derivatised polysaccharides; polyphosphazenes; biodegradable microspheres; monophosphoryl lipid A and quil A. Cytokines, such as GM-CSF or interleukin-2, -7, or -12, and chemokines may also be used as adjuvants.

In one embodiment, the adjuvants may include, for example, a combination of monophosphoryl lipid A, such as 3-de-O-acylated monophosphoryl lipid A (3D-MPL) together with an aluminium salt.

In place of 3D-MPL, other toll like receptor 4 (TLR4) ligands such as aminoalkyl glucosaminide phosphates (WO 98/50399, WO 01/34617 and WO 03/065806) may be used.

In one embodiment, the adjuvant may include a TLR9 agonist such as an immunostimulatory oligonucleotide comprising unmethylated CpG, for example:

SEQ ID NO: 6102

TCC ATG ACG TTC CTG ACG TT (CpG 1826)

SEQ ID NO: 6103

TCT CCC AGC GTG CGC CAT (CpG 1758)

SEQ ID NO: 6104

ACC GAT GAC GTC GCC GGT GAC GGC ACC ACG

SEQ ID NO: 6105

TCG TCG TTT TGT CGT TTT GTC GTT (CpG 2006, CpG
7909)

SEQ ID NO: 6106

TCC ATG ACG TTC CTG ATG CT (CpG 1668)

In one embodiment of the present invention, the adjuvant comprises the combination of a CpG-containing oligonucleotide and a saponin derivative, for example the combination of CpG and QS21 (WO 00/09159 and WO 00/62800).

The adjuvant formulation may additionally comprise an oil in water emulsion and/or tocopherol.

In one embodiment, the adjuvant comprises a saponin, for example QS21 (Aquila Biopharmaceuticals Inc., Framingham, Mass.), that may be used alone or in combination with other adjuvants. In one embodiment, the adjuvant comprises the combination of a monophosphoryl lipid A and saponin derivative, such as the combination of QS21 and 3D-MPL (WO 94/00153), or a composition where the QS21 is quenched with cholesterol (WO 96/33739).

In one embodiment, the adjuvant components are provided in an oil-in-water emulsion and tocopherol. In one embodiment, the adjuvant formulation comprises QS21, 3D-MPL and tocopherol in an oil-in-water emulsion (WO 95/17210).

In another embodiment, the adjuvants may be formulated in a liposomal composition.

In an embodiment, the adjuvant system comprises a CpG oligonucleotide, 3D-MPL and QS21 either presented in a liposomal formulation or an oil in water emulsion (WO 95/17210).

The amount of CpG or immunostimulatory oligonucleotides in the adjuvants or immunotherapeutics of the present invention is generally small, but depending on the immunotherapeutic formulation may be in the region of 1-1000 μg per dose, for example 1-500 μg per dose.

The amount of saponin for use in the adjuvants of the present invention may be in the region of 1-1000 μg per dose, for example 1-500 μg per dose, such as 1 to 100 μg per dose, particularly 25, 30, 40, 50, 60, 70, 80 or 90 μg per dose.

Generally, it is expected that each human dose will comprise 0.1-1000 μg of antigen, preferably 0.1-500 μg, preferably 0.1-100 μg, most preferably 0.1 to 50 μg. An optimal amount can be ascertained by standard studies.

Following initial administration or vaccination, subjects may receive one or several booster administrations or immunisations adequately spaced.

Other adjuvants that may be used include Montanide ISA 720 (Seppic, France), SAF (Chiron, Calif., United States), ISCOMS (CSL), MF-59 (Chiron), Ribi Detox, R^{C}-529 and other aminoalkyl glucosaminide 4-phosphates (AGPs) (GSK, Hamilton, Mont.).

In one embodiment, the adjuvant may comprise one or more of 3D-MPL, QS21 and an immunostimulatory CpG oligonucleotide. In an embodiment all three immunostimulants are present. In another embodiment 3D-MPL and QS21 are presented in an oil in water emulsion, and in the absence of a CpG oligonucleotide.

A composition for use in the method of the present invention may comprise a pharmaceutical composition comprising tumour associated antigen as described herein, or a fusion protein, in a pharmaceutically acceptable excipient.

In a further aspect the invention provides a method of treating a responder patient with an appropriate therapy, for example immunotherapy, for example cancer immunotherapy such as cancer testis immunotherapy, after identification of the same as a responder thereto.

Thus, the invention provides a method of treating a patient comprising the step of administering a therapeutically effective amount of a therapy, for example immunotherapy as described herein, for example cancer immunotherapy, such as MAGE antigen specific immunotherapy, after first characterising the patient as a responder based on differential expression of at least one immune activation gene, for example as shown by appropriate analysis of a sample derived from the patient. In particular wherein the patient is characterised as a responder based on one or more embodiments described herein.

In one aspect the immunotherapy comprises an appropriate adjuvant, as described herein.

In yet a further embodiment of the invention there is provided a method of treating a patient suffering from, for example, a MAGE expressing tumour, the method comprising determining whether the patient expresses the gene signature of the invention and then administering, for example, a therapy as described herein, for example MAGE specific immunotherapy. In a further embodiment, the patient is treated with, for example, the MAGE specific immunotherapy to prevent or ameliorate recurrence of disease, after first receiving treatment such as resection by surgery of any tumour or other chemotherapeutic or radiotherapy treatment.

A further aspect of the invention is a method of treating a patient suffering from a MAGE expressing tumour, the method comprising determining whether the patient's tumour expresses a profile according to any embodiment of the invention from a biological sample given by a patient and then administering a MAGE specific immunotherapeutic to said patient.

Also provided is a method of treating a patient susceptible to recurrence of MAGE expressing tumour having been treated to remove/treat a MAGE expressing tumour, the method comprising determining whether the patient's tumour expresses one or more genes selected from any embodiment of the invention from a biological sample given by a patient and then administering a MAGE specific immunotherapeutic.

In one embodiment, the invention also provides a method of treatment or use employing, comprising or consisting of:
a MAGE specific immunotherapeutic comprising a MAGE antigen or peptide thereof,
a MAGE antigen comprising a MAGE-A3 protein or peptide as described herein,
a MAGE antigen comprising the peptide EVDPIGHLY or other epitopes as described herein,
a MAGE antigen or peptide fused or conjugated to a carrier protein, for example in which the carrier protein is selected from protein D, NS1 or CLytA or fragments thereof as described herein, and/or
a MAGE specific immunotherapeutic further comprising an adjuvant, for example in which the adjuvant comprises one or more or combinations of: 3D-MPL; aluminium salts; CpG containing oligonucleotides; saponin-containing adjuvants such as QS21 or ISCOMs; oil-in-water emulsions; and liposomes.

The invention also extends to use of an immunotherapy such as a cancer immunotherapy, in particular MAGE immunotherapy in the manufacture of a medicament for the treatment of a patient such as a cancer patient designated as a responder, thereto.

The inventors believe that it may be possible to induce a responders profile in at least some non-responders, for example by subjecting the patient to radiation therapy, or administering an inflammatory stimulant such as interferon or a TLR 3 (for example as described in WO 2006/054177), 4, 7, 8 or TLR 9 agonist (for example containing a CpG motif, in particular administering a high dose thereof such as 0.1 to 75 mg per Kg adminstered, for example weekly).

The high dose of CpG may, for example be inhaled or given subcutaneously.

The invention further provides the use of MAGE specific immunotherapy in the manufacture of a medicament for the treatment of patients suffering from MAGE expressing tumour or patients who have received treatment (e.g. surgery, chemotherapy or radiotherapy) to remove/treat a MAGE expressing tumour, said patient expressing the gene signature of the invention.

The immunotherapy may then be administered to for example responders or once the responders profile has been induced.

In one aspect the invention provides use of MAGE specific immunotherapy in the manufacture of a medicament for the treatment of patients suffering from a MAGE expressing tumour, said patient characterised by their tumour expressing one or more genes selected from any embodiment of the invention.

The invention also provides use of MAGE specific immunotherapy in the manufacture of a medicament for the treatment of patients susceptible to recurrence from MAGE expressing tumour said patient characterised by their tumour one or more genes selected from any embodiments of the invention.

Advantageously, the invention may allow treatment providers to target those populations of patients that will obtain a clinical benefit from receiving an appropriate immunotherapy. It is expected that after screening that at least 60% of patients such as 70, 75, 80, 85% or more of patients deemed/characterised as responders will receive a clinical benefit from the immunotherapy, which is a significant increase over the current levels observed with therapy such as cancer therapy generally.

Advantageously if the cancer immunotherapy is given concomitantly or subsequent to chemotherapy it may assist in raising the patient's immune responses, which may have been depleted by the chemotherapy.

In a further embodiment the immunotherapy may be given prior to surgery, chemotherapy and/or radiotherapy.

Antigen Specific Cancer Immunotherapeutics (ASCIs) suitable for use in the invention may, for example include those capable of raising a MAGE specific immune response. Such immunotherapeutics may be capable of raising an immune response to a MAGE gene product, for example a MAGE-A antigen such as MAGE-A3. The immunotherapeutic will generally contain at least one epitope from a MAGE gene product. Such an epitope may be present as a peptide antigen optionally linked covalently to a carrier and optionally in the presence of an adjuvant. Alternatively larger protein fragments may be used. For example, the immunotherapeutic for use in the invention may comprise an antigen that corresponds to or comprises amino acids 195-279 of MAGE-A1. The fragments and peptides for use must however, when suitably presented be capable of raising a MAGE specific immune response. Examples of peptides that may be used in the present invention include the MAGE-3.A1 nonapeptide EVDPIGHLY [SEQ ID NO: 6094], and the following MAGE-A3 peptides:

| | |
|---|---|
| FLWGPRALV; | SEQ ID NO: 6095 |
| MEVDPIGHLY; | SEQ ID NO: 6096 |
| VHFLLLKYRA; | SEQ ID NO: 6097 |
| LVHFLLLKYR; | SEQ ID NO: 6098 |
| LKYRAREPVT; | SEQ ID NO: 6099 |
| ACYEFLWGPRALVETS; | SEQ ID NO: 6100 |
| and | |
| TQHFVQENYLEY; | SEQ ID NO: 6101 |

Alternative ASCIs include cancer testis antigens such as PRAME, LAGE 1, LAGE 2, and others.

In one embodiment of the present invention, the antigen to be used may consist or comprise a MAGE tumour antigen, for example, MAGE 1, MAGE 2, MAGE 3, MAGE 4, MAGE 5, MAGE 6, MAGE 7, MAGE 8, MAGE 9, MAGE 10, MAGE 11 or MAGE 12. The genes encoding these MAGE antigens are located on chromosome X and share with each other 64 to 85% homology in their coding sequence (De Plaen, 1994). These antigens are sometimes known as MAGE A1, MAGE A2, MAGE A3, MAGE A4, MAGE A5, MAGE A6, MAGE A7, MAGE A8, MAGE A9, MAGE A 10, MAGE A11 and/or MAGE A12 (The MAGE A family). In one embodiment, the antigen is MAGE A3.

In one embodiment, an antigen from one of two further MAGE families may be used: the MAGE B and MAGE C group. The MAGE B family includes MAGE B1 (also known as MAGE Xp1, and DAM 10), MAGE B2 (also known as MAGE Xp2 and DAM 6) MAGE B3 and MAGE B4—the MAGE C family currently includes MAGE C1 and MAGE C2. In general terms, a MAGE protein can be defined as containing a core sequence signature located towards the C-terminal end of the protein (for example with respect to MAGE A1 a 309 amino acid protein, the core signature corresponds to amino acid 195-279).

In one embodiment the MAGE antigen may comprise the full length MAGE protein. In an alternative embodiment the MAGE antigen may comprise amino acids 3 to 312 of the MAGE antigen.

In alternative embodiments the MAGE antigen may comprise 100, 150, 200, 250 or 300 amino acids from the MAGE protein, provided that the antigen is capable of generating an immune response against MAGE, when employed in an immunotherapeutic treatment.

As far as the inventors are aware it has never been proposed to exclude gender related genes from gene profiles. Therefore in a further aspect the invention provides a method of generating a gene profile, wherein at one stage in the analysis the gender related genes are excluded.

In one aspect the gender related genes may be excluded, for example at the initial stages of the analysis by simply removing the relevant genes from the raw data.

In another aspect the gender related genes may removed from a gene list that has been generated by a statistical method, for example as described herein, by performing a multivariant analysis.

In another aspect the gender related aspects can be removed from the analysis by simply ensuring that the same number of males and females are included for each category under analysis, as appropriate.

Use of the word “comprising” in the context of this specification in intended to be non-limiting ie means “including”.

Embodiments are specifically envisaged where aspects of the invention comprising a certain element or elements are limited to said aspects consisting or consisting essentially of the relevant elements as separate embodiments.

The examples below are shown to illustrate the methodology, which may be employed to prepare particles of the invention.

Discussion of documents in this specification is intended to give context to the invention and aid understanding of the same. In no way is it intended to be an admission that the document or comment is known or is common general knowledge in the relevant field.

### EXAMPLES

### Example 1

### MAGE008 MAGE Melanoma Clinical Trial

In this on-going trial, the recMAGE-A3 protein (recombinant protein D—Mage Fusion protein) is combined with two different immunological adjuvants: either AS02B (QS21, MPL) or AS15 (QS21, MPL and CpG7909). The objectives were to discriminate between the adjuvants in terms of safety profile, clinical response and immunological response.

In this experiment two adjuvant compositions are made up of mixtures of two immunostimulants:
1. QS21 (Purified, naturally occurring saponin molecule from the South-American tree Quillaja Saponaria Molina), and
2. MPL (3 de-O-acetylated monophosphoryl lipid A—detoxified derivative of lipid A, derived from S. minnesota LPS).
   AS02B is an oil-in-water emulsion of QS21 and MPL.

In animal models these adjuvants have been successfully shown to induce both humoral and TH 1 types of cellular-mediated immune responses, including CD4 and CD8 T-cells producing IFNα (Moore et al., 1999; Gérard et al., 2001). Moreover, the injection of recombinant protein formulated in this type of adjuvant leads to the induction of a systemic anti-tumour response: indeed, vaccinated animals were shown to be protected against challenges with murine tumour cells genetically engineered to express the tumour antigen, and regressing tumours were shown to be highly infiltrated by CD8, CD4 and NK cells and by macrophages.

The second adjuvant system is AS15: it contains a third immunostimulant, namely CpG7909 (otherwise known as CpG 2006 supra), in addition to MPL and QS21, in a liposome formulation.

### Study Overview

1. Design
   The MAGE008 trial is:
open
randomized
two-arm (AS02B vs. AS15)
with 68 patients in total.

As described above, the recMAGE-A3 protein is combined with either AS02B or AS15 adjuvant system.
2. Patient population

The recMAGE-A3 protein is administered to patients with progressive metastatic melanoma with regional or distant skin and/or lymph-node lesions (unresectable stage III and stage IV M1a). The expression of the MAGE-A3 gene by the tumour was assessed by quantitative PCR. The selected patients did not receive previous treatment for melanoma (recMAGE-A3 is given as first-line treatment) and had no visceral disease.
3. Schedule of immunization

### Method of Treatment Schedules

### Adjuvant Setting

The method of treatment schedule for use in disease in an adjuvant (post-operative) setting may comprise administration of an antigen as described herein according to the following schedules:

Administration of antigen at three week intervals for the first 5 to 8 vaccinations, followed at 3 month intervals for the next 8, 9 or more vaccinations.

The antigen may be administered at the exact time frame indicated, or the antigen may be given 1, 2, 3 or 4 days before or after the exact interval, as required or as practical. An example of this schedule is shown in the table below:
Induction: 5 vaccinations at intervals of 3 weeks for example Weeks 0, 3, 6, 9, 12 or Weeks 0, 6, 9, 12
Maintenance: 9 vaccinations at intervals of 3 months
   Alternatively, the vaccinations may be given initially at 2 week intervals, for example 6 injections at two week intervals followed by appropriate maintenance therapy.

### Active Disease

The method of treatment schedule for use in active or unresectable disease, for example in melanoma cancer, comprising: administration of an antigen as described herein at two or three week intervals for the first six months to one year of treatment. A schedule may comprise the following pattern of injections: the antigen may be given at two week intervals for the first 4 to 10 vaccinations, followed by 3 week intervals for the next 4 to 10 vaccinations, then at 6 week intervals for the next 3 to 7 vaccinations. Long term treatment may then continue with vaccinations at 3 month intervals for 3 to 5 vaccinations, followed by 6 month intervals for the next 3 to 5 vaccinations.

The antigen may be administered at the exact time frame indicated, or the antigen may be given 1, 2, 3 or 4 days before or after the exact interval, as required or as practical. An example of this schedule is shown in the table below:
Cycle 1: 6 vaccinations at intervals of 2 weeks (Weeks 1, 3, 5, 7, 9, 11)
Cycle 2: 6 vaccinations at intervals of 3 weeks (Weeks 14 or 15, 17 or 18, or 21, 23 or 24, 26 or 27, 29 or 30)
Cycle 3: 4 vaccinations at intervals of 6 weeks (Weeks 33 or 34, 39 or 40, 45 or 46, 51 or 52)
Long Term Treatment: 4 vaccinations at intervals of 3 months, for example followed by 4 vaccinations at intervals of 6 months

For both of the above treatment regimes additional vaccinations may be given after treatment, as required.

In order to screen potential participants in the above clinical trial tumour biopsies were received (both prior to any immunization and after immunization, if applicable, as relapses), as frozen tumour samples. Nevertheless relapse samples and samples for individuals who did not complete the first cycle of treatment were not included in the analysis to generate the genes list of the invention including in the examples, unless stated otherwise). From these samples RNA was extracted for quantitative PCR. The quality of this purified RNA was extremely high and it was suitable for microarray analysis. Tumour samples were therefore analyzed by microarray. The goal was to identify in pre-vaccination biopsies a set of genes associated with the clinical response and to develop a mathematical model that would predict patient clinical outcome, so that patients likely to benefit from this antigen-specific cancer immunotherapeutic are properly identified and selected. Gene profiling was performed only on biopsies from patients who signed the informed consent for microarray analysis.

### Materials and Methods

### Tumour Specimens

96 tumour specimens (both pre-vaccination and after vaccination types) were used from the MAGE008 MAGE-3 melanoma clinical trial. These were fresh frozen preserved in the RNA stabilizing solution RNAlater.

### RNA Purification

Tumoural total RNA was purified using the Tripure method—Tripure extraction (Roche Cat. No. 1 667 165). The protocols provided were followed subsequently by the use of an RNeasy Mini kit—clean-up protocol with DNAse treatment (Qiagen Cat. No. 74106).

### RNA Quality Control

Quantification of RNA was initially completed using optical density at 260 nm and Quant-IT RiboGreen RNA assay kit (Invitrogen—Molecular probes R11490). The quality of the 28s and 18s ribosomal RNA peaks was assessed by use of the Agilent bioanalyser.

### RNA Labeling and Amplification for Microarray Analysis

Due to the small biopsy size received during the clinical study, an amplification method was used in conjunction with the labelling of the RNA for microarray analysis, the Nugen 3′ ovation biotin kit (Labelling of 50 ng of RNA—Ovation biotin system Cat; 2300-12, 2300-60). A starting input of 50 ng of total RNA was used.

### Microarray Chips

The Affymetrix HU-U133.Plus 2.0 gene chips were utilized. These chips cover about 47,000 potential gene transcripts.

### Microarray Hybridization and Scanning

The hybridized chips were washed and scanned according to the standard Affymetrix protocols.

### Data Analysis:

All calculations were run under R 2.4.0 program (R Development Core Team (2006). R: A language and environment for statistical computing. R Foundation for Statistical Computing, Vienna, Austria. ISBN 3-900051-07-0.

### Data Processing & Normalization

The fluorescent scanned data were then normalized using GCRMA using all 96 patient sample data to allow comparisons between individual chips. The following references describe this method: Jean (ZHIJIN) Wu and Rafael Irizarry with contributions from James MacDonald Jeff Gentry (2005). GCRMA: Background Adjustment Using Sequence Information. R package version 2.4.1.; Wu Z, Irizarry R A, Gentleman R, Martinez-Murillo F, Spencer F: A model-based background adjustment for oligonucleotide expression arrays. Journal of the American Statistical Association 2004, 99:909-917.

### Patient Sample Stratification

Two data sets were used for the selection of the set of genes correlating with clinical response and the subsequent predictive model developments:
Data related to patients having received the AS15 adjuvant, and not AS02B. 23 individual patient data fall under this category, 7 of them are responding to the antigen specific immunotherapeutic (ASCI), and the remaining 16 are not responding to the ASCI.
Data related to patients having received the AS15 adjuvant and data related to patients having received the AS02B adjuvant and responding to the ASCI treatment. Four Responder individual data are added in this stratification, bringing the total number of available patients for model development to 27. Among them, 11 Responders and 16 Non-Responders. This second stratification has this advantage over the first one to have a higher sample size and more balanced experimental groups.

Moreover, three categories of clinical response exist: objective response, stable disease, and mixed response. Only the first two categories are taken into account for these data stratification, as being internationally recognized. The mixed response category of patients, for which some melanoma lesion will regress while other will keep on progressing, as being heterogeneous, is not taken into account. Mixed Responders are kept away form the data stratifications.

### Calculation of Differential Expression

To identify in pre-vaccination biopsies a set of genes that is associated with the clinical response, differential expression between patients responding to ASCI and patients not responding to ASCI was calculated using the Signal-To-Noise statistics (S2N): for each microarray probe set, difference in experimental group mean is divided by the sum of experimental group standard deviations (SD). Values are then transformed as absolute values.

### Gene Profile Normalization

To make the probe sets having low and high levels of expression comparable and to put an emphasis on differential profiles rather than absolute profiles, data was further normalized at the probe set level. Z-score normalization was used, where each probe set measure is subtracted by its mean over samples and divided by its SD.

### Example 2

### Predictive Model

The kNN machine learning algorithm (or predictive rule), knn function, interfaced in class package (Venables, W. N. & Ripley, B. D. (2002) Modern Applied Statistics with S. Fourth Edition. Springer, N.Y. ISBN 0-387-95457-0) was used to train clinical outcome predictive models and to predict the MAGE008 patient clinical outcomes, under reporter lists calculated by the differential expression and gene normalization processes. A value of 5 was used for k parameter.

Several feature list sizes were employed to develop the predictive model. Feature lists of 2, 4, 6, 8, 10, 12, 14, 16, 18, 20, 22, 26, 30, 40, 50, 65, 80, 100, 120, 140, 160, 180, 200, 250, 300, 400, 500, 750, 1000, 2000, 4000, 8000 and 16000 probe sets were worked out. The feature lists were obtained by taking the above mentioned number of probe sets in the probe set matrix ranked in descending order according to their S2N value.

A Leave-one-out (LOO) scheme was used for cross-validation of all feature list size classifiers, with re-calculation of reporter list at each cross-validation loop, to evaluate the predictive model performance.

Sensitivity (Se) and Positive Predictive Value (PPV) were used as performance indicators. Se is defined as the proportion of true positives (TP) among Responders, and PPV is defined as the proportion of true positives (TP) among patients predicted as Responders. Se and PPV measures are preferred as model performance indicators since
Se is relative to the responding patients prediction accuracy, the highest accuracy is searched for in this model;
PPV is relative to the vaccine efficacy in the predicted population of responding patients, the highest vaccine efficacy in this population is searched for, given the highest accuracy in predicting responding patients.

The final predictive model is the feature list size classifier showing the highest Se combined to the highest PPV under the shortest feature list.

### Results

Predictive Model and Associated Gene List Correlating with Treatment Clinical OUTCOME Based on AS15 only Patients.

FIG. 1 illustrates the predictive model development results under the first stratification of patient data (AS15 receiving patients only). 18 genes (probe sets) are sufficient to predict the clinical outcome of patients at best 86% Sensitivity and 86% Positive Predictive Value, as estimated by cross-validation and under a kNN predictive rule. In other words, 18 genes can correctly predict 86% of Responders (6 Responders out of 7) and the anticipated vaccine treatment efficacy under this predictive model for selection of patients is anticipated to be 86%. Selecting the patients under this model nearly triples the asci efficacy (to compare to efficacy without patient selection, 7 dived by 23-30%).

The 18 probe sets, and associated gene names, necessary for this model prediction is given in table 1.

Implementaion of the 18-probe set predictive model, predicting the clinical response of further patient samples:

The following R code chunk in a R 2.4.0 session predicts the clinical response of further samples.

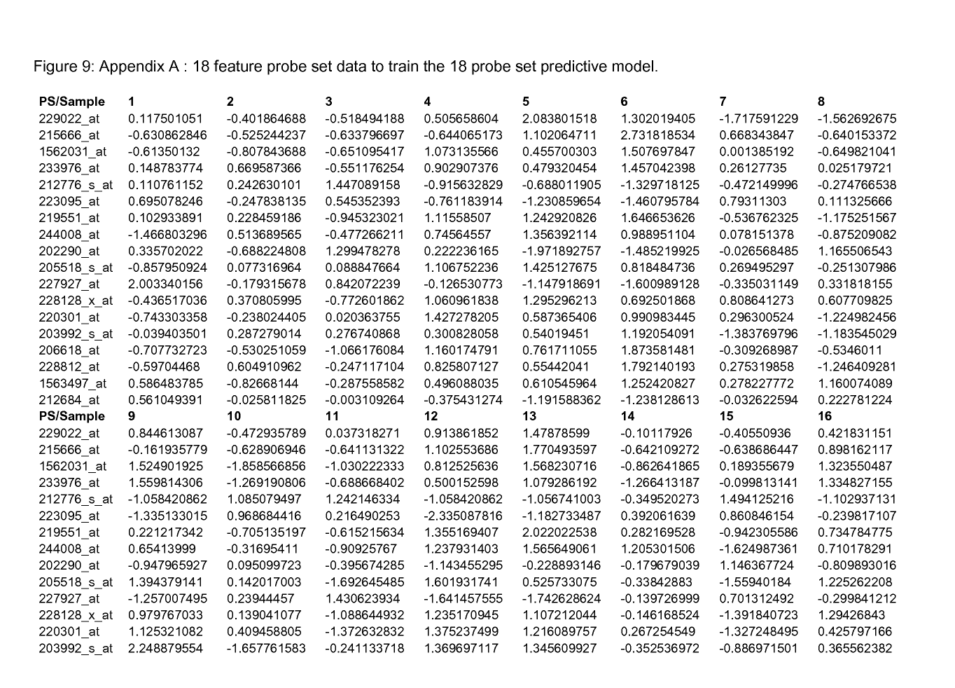

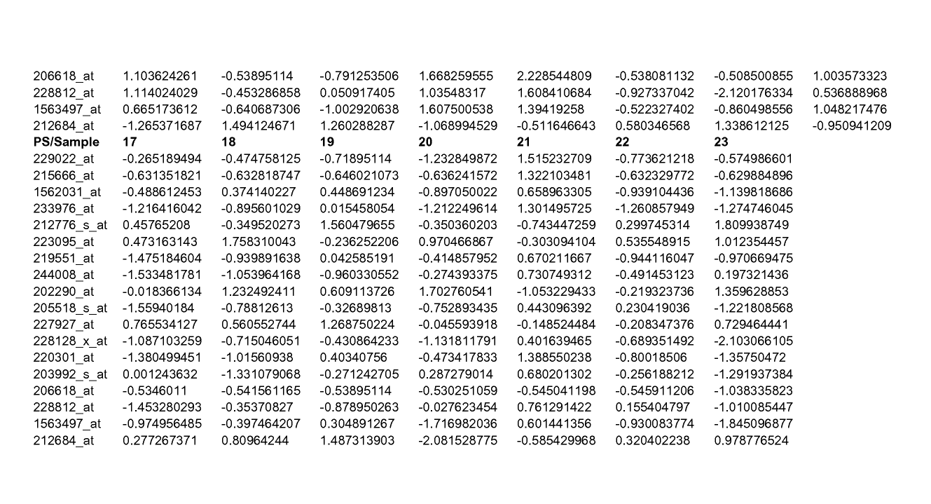

library(class)
knn(t(train), t(test), k=5)
where
train is an object (class matrix) of 18 rows and 23 columns to be built from Appendix A table (FIG. 9)
test is an object of class matrix containing the 18 feature probe sets of table 1 as rows and expression data (GCRMA processed and Z-score normalized) of the further samples to predict as columns.

Probe set order of test has to be identical to train, and Z-score calculation has to be computed for each gene using the medians (MeTr) and standard deviations (SdTr) depicted in the following table:

| PS | MeTr | SdTr |
|---|---|---|
| 229022_at | 7.185521739 | 0.548746246 |
| 215666_at | 3.588173913 | 2.045094144 |
| 1562031_at | 6.309826087 | 1.569395299 |
| 233976_at | 3.233869565 | 0.720041117 |
| 212776_s_at | 3.822130435 | 1.190575961 |
| 223095_at | 10.65008696 | 1.122050716 |
| 219551_at | 4.694434783 | 1.657036534 |
| 244008_at | 4.537826087 | 1.409750096 |
| 202290_at | 6.018434783 | 0.731497581 |
| 205518_s_at | 6.248304348 | 1.561050067 |
| 227927_at | 6.580826087 | 1.136688598 |
| 228128_x_at | 6.386434783 | 1.945937302 |
| 220301_at | 6.067347826 | 1.652552505 |
| 203992_s_at | 5.357173913 | 0.664253487 |
| 206618_at | 3.495478261 | 1.14941451 |
| 228812_at | 7.263391304 | 1.426009363 |
| 1563497_at | 6.098173913 | 1.537682898 |
| 212684_at | 8.69373913 | 0.880957858 |

### FIG. 1 Legend.

X-axis, number of probe sets used as features of the predictive model, in increasing order and log scale. Y-axis, 5NN predictive model performances, Se (emptycircles) and PPV (solid dots) measures. The horizontal dashed-line is the basal vaccine treatment efficacy not taking into account predictive model based patient selection. The vertical dash-line indicates the classification optimum, i.e. the smallest feature list size for which the Se is maximum associated to the maximum PPV given this highest Se.

Table 1 shows Affymetrix probe sets (18) used as 5NN model features for predictions of clinical outcome trained on first data stratification.

### Example 3

Predictive model and associated gene list correlating with treatment clinical outcome based on responding patients to one of the following treatments:

ASCI and AS15 (a liposomal formulation of MPL, QS21 and CpG), and

ASCI and AS02B (an oil in water formulation of MPL and QS21).

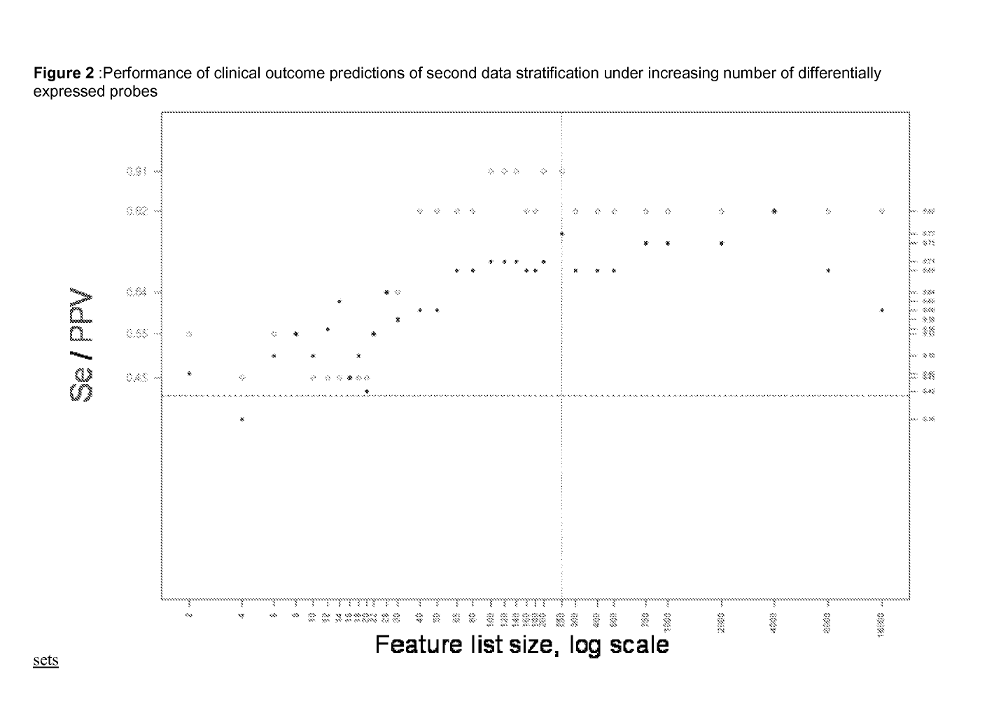

FIG. 2 illustrates the predictive model development results under the second stratification of patient data AS15 receiving patients and AS02B receiving patients showing a clinical response to treatment). 250 genes (probe sets) are required to predict the clinical outcome of patients at best 91% Sensitivity and 77% Positive Predictive Value (LOO cross-validation estimates). In other words, 250 genes can correctly predict 91% of Responders (10 Responders out of 11), and the anticipated vaccine treatment efficacy under this predictive model for selection of patients is anticipated to be 77%. Selecting the patients under this model nearly doubles the ASCI efficacy (to compare to efficacy without patient selection, 11 dived by 27-41%).

The 250 probe sets, and associated gene names, necessary for this model prediction is given in Table 2.

Implementation of the 250-probe set predictive model, predicting the clinical response of further patient samples:

The following R code chunk in a R 2.4.0 session predicts the clinical response of further samples.

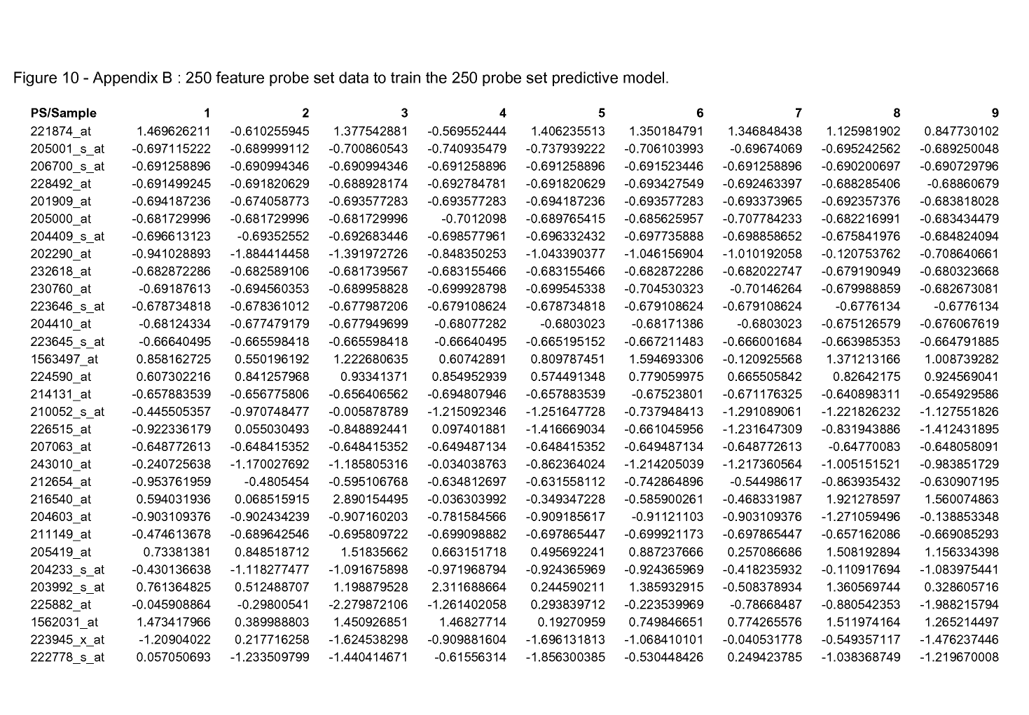

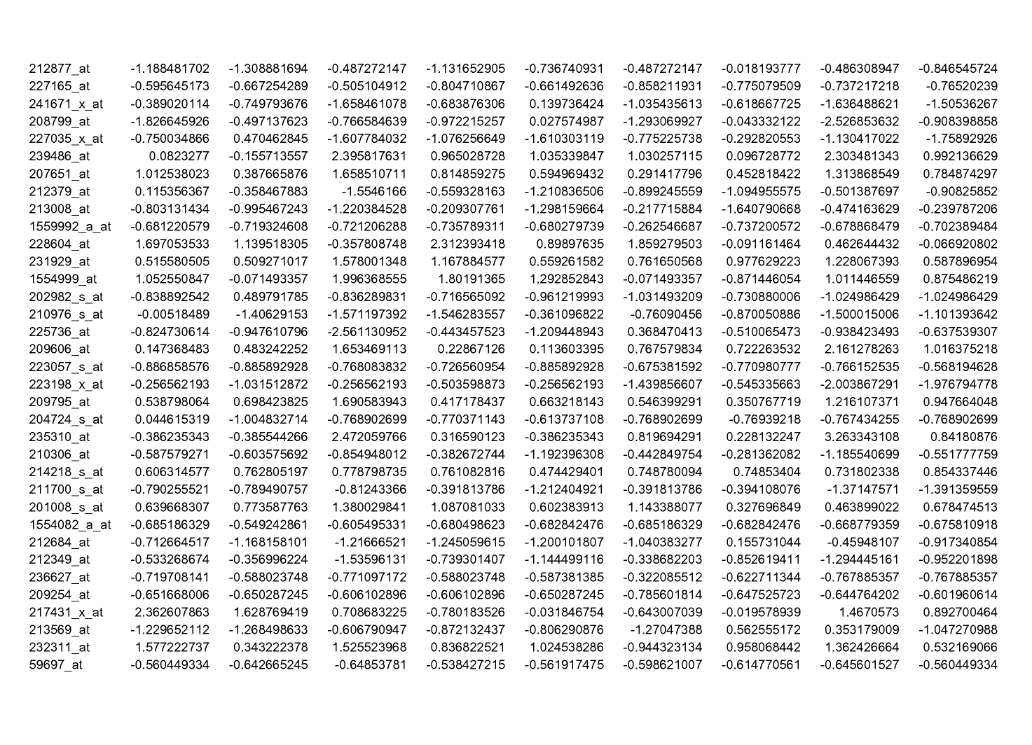

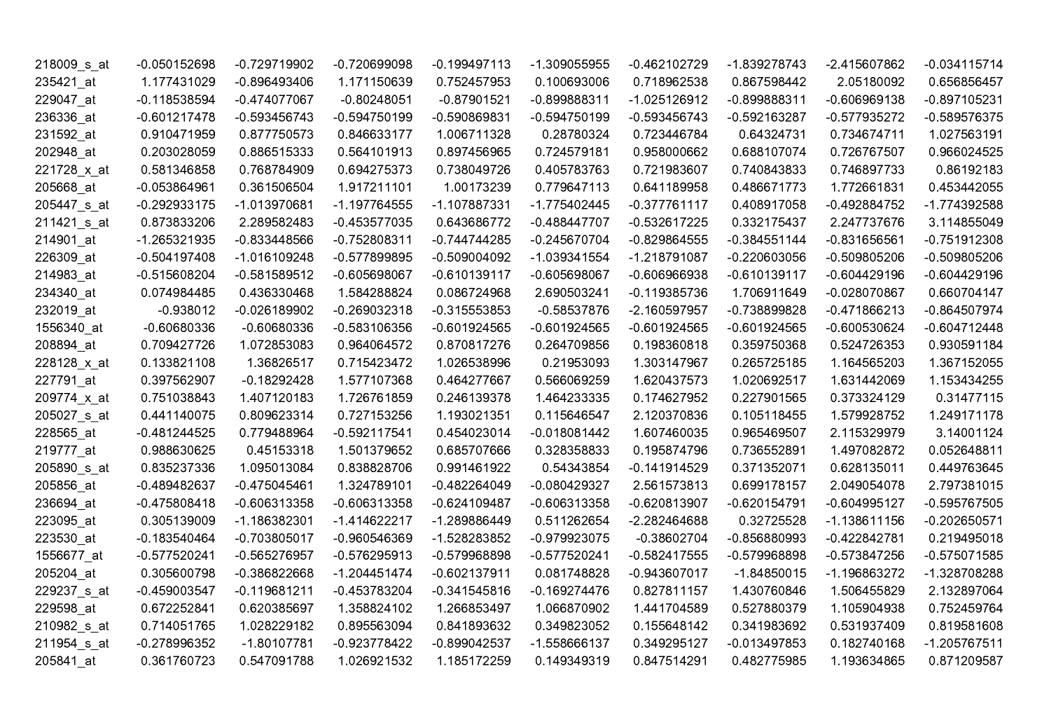

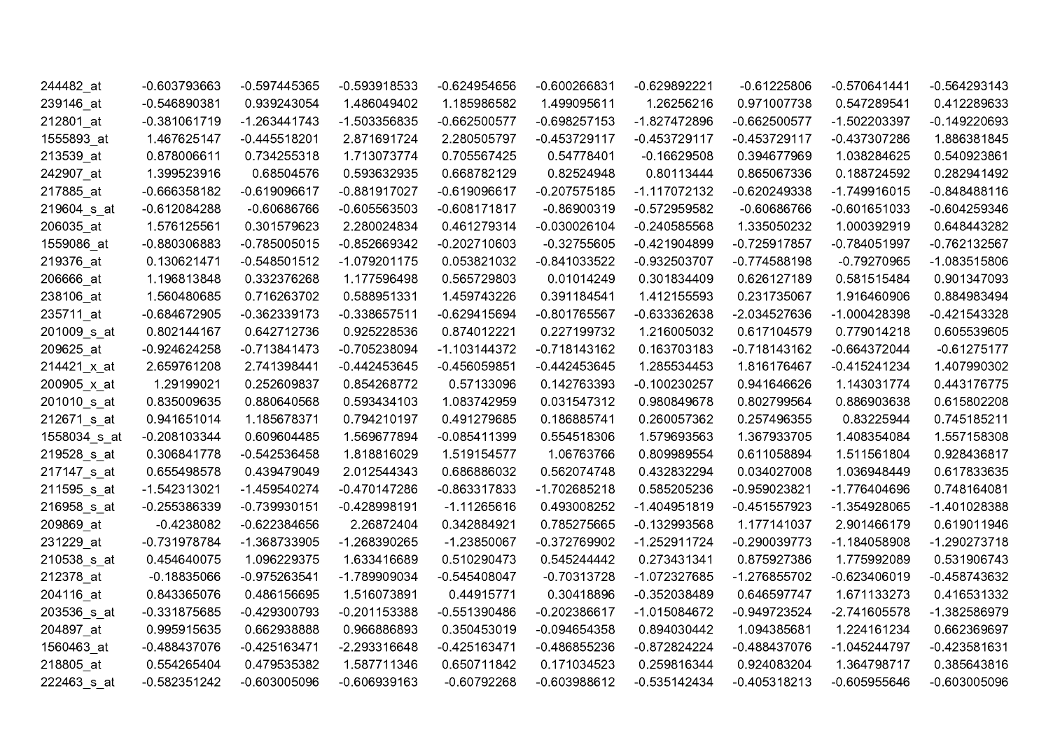

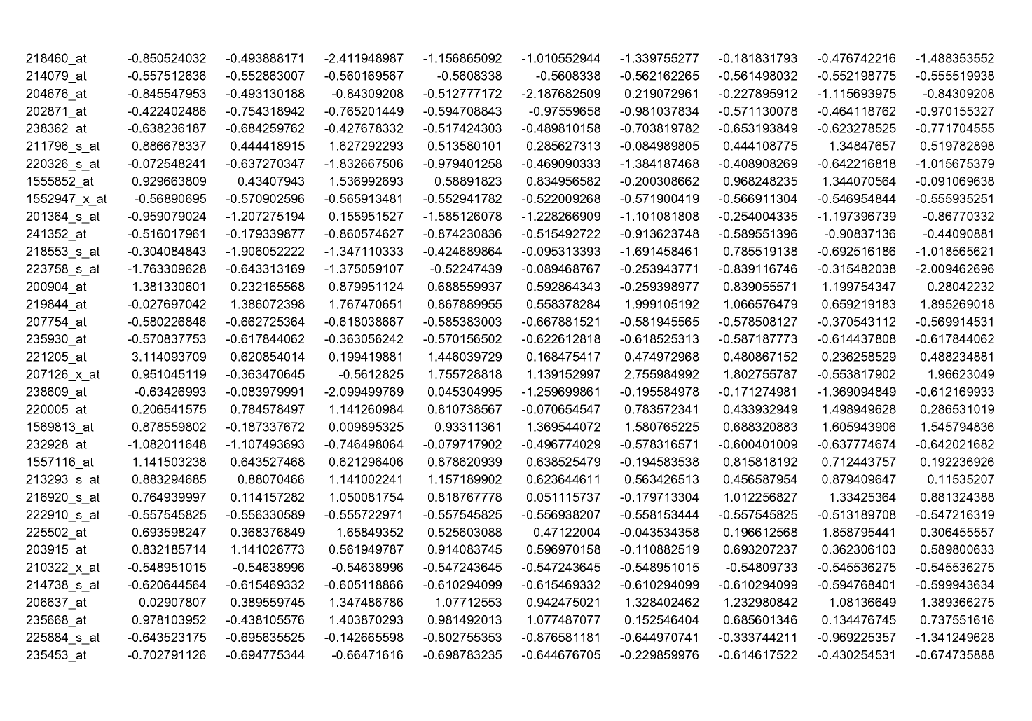

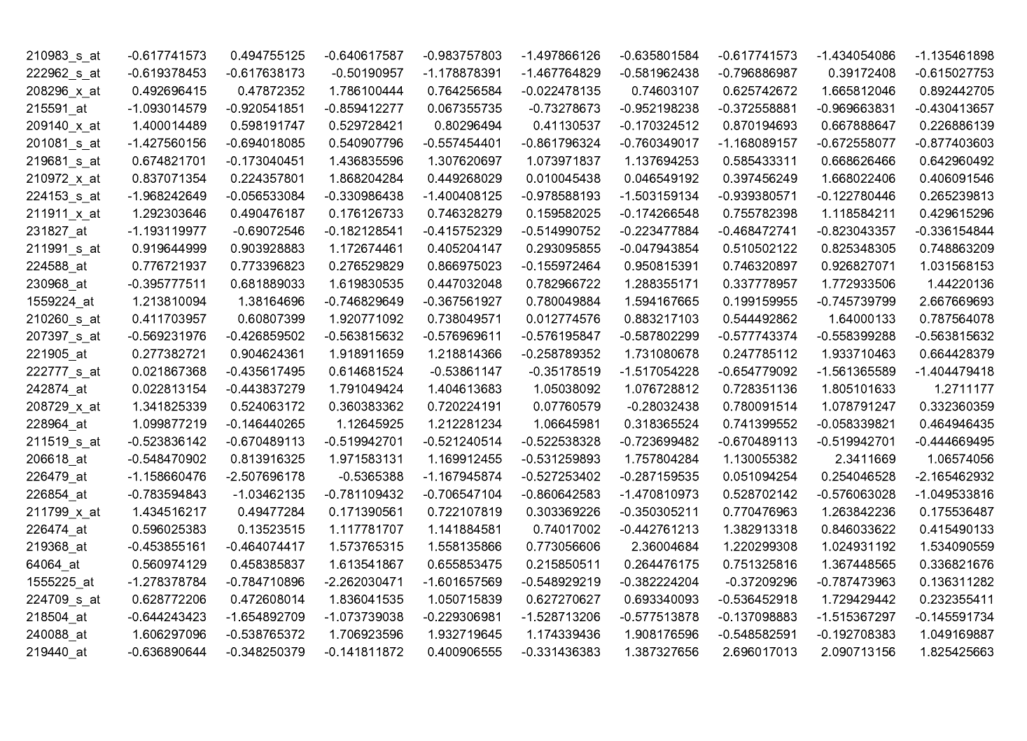

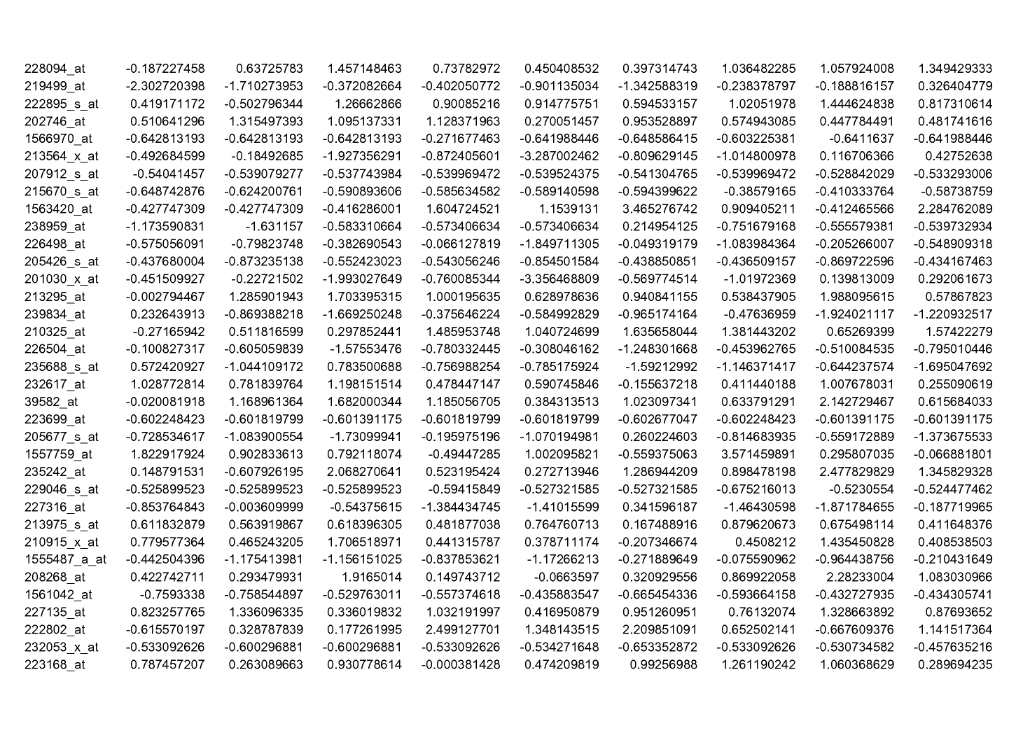

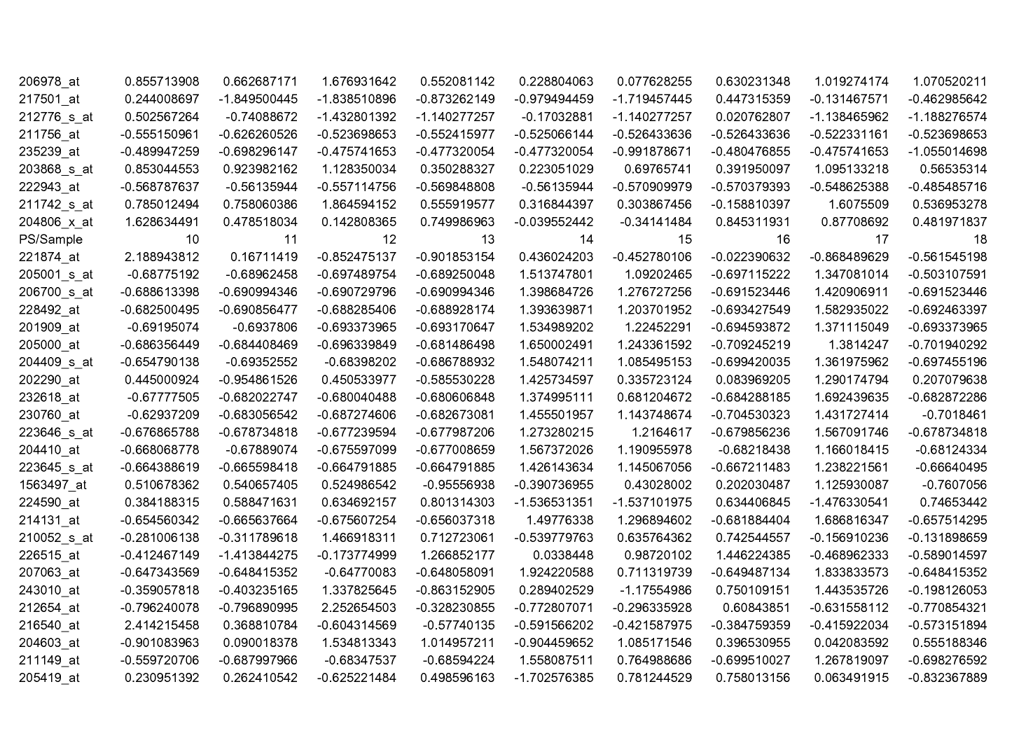

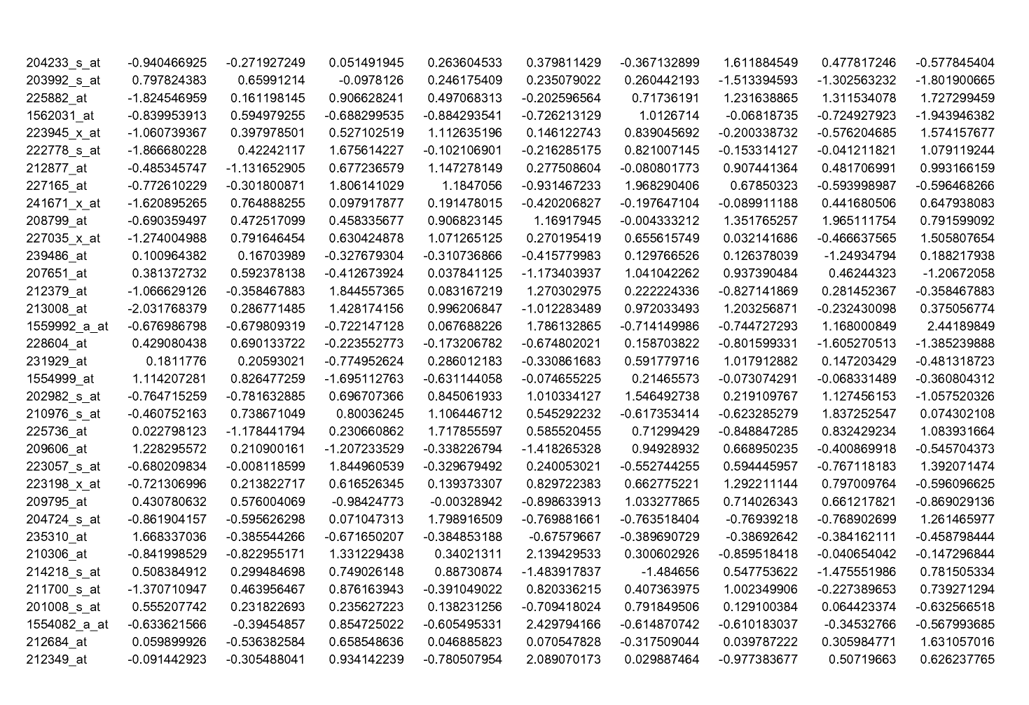

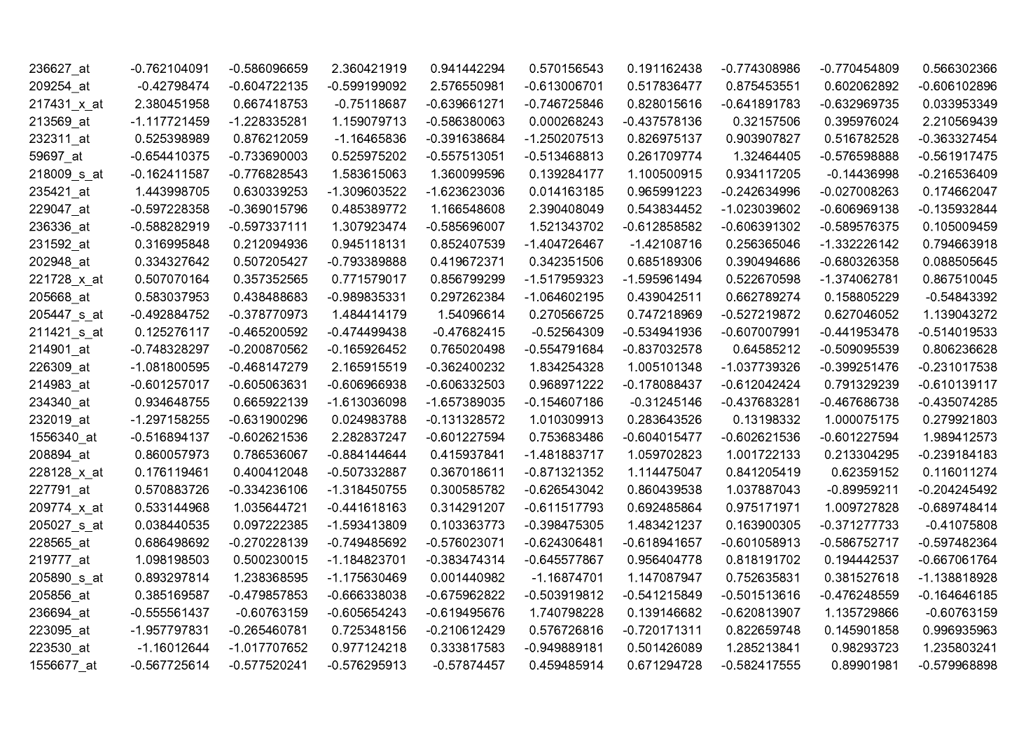

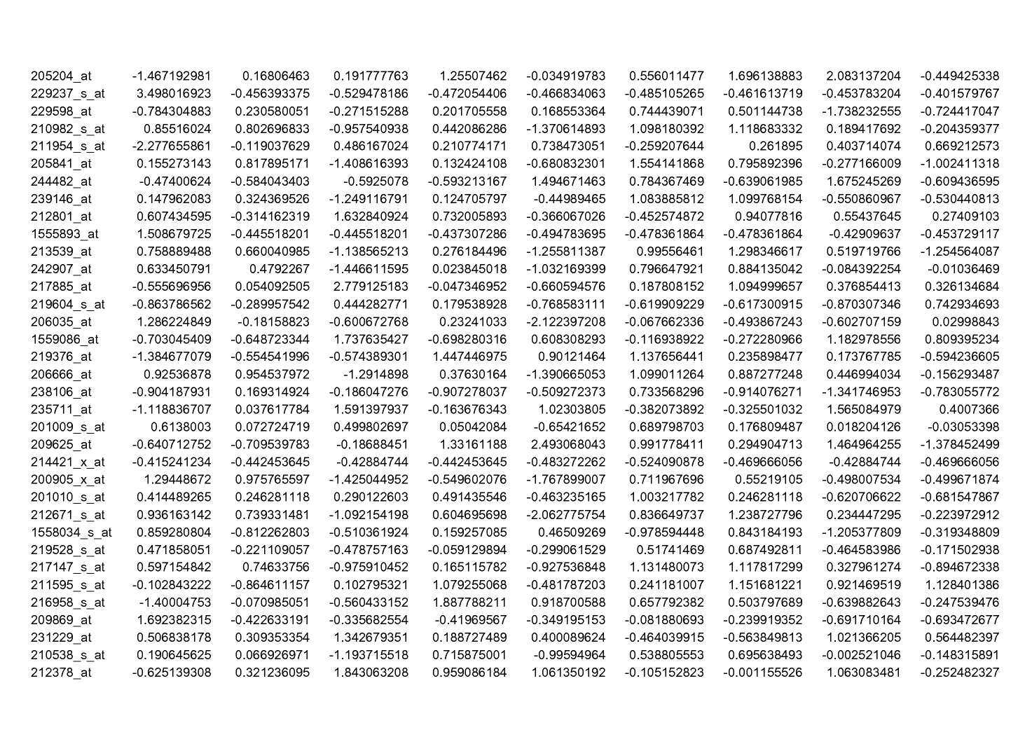

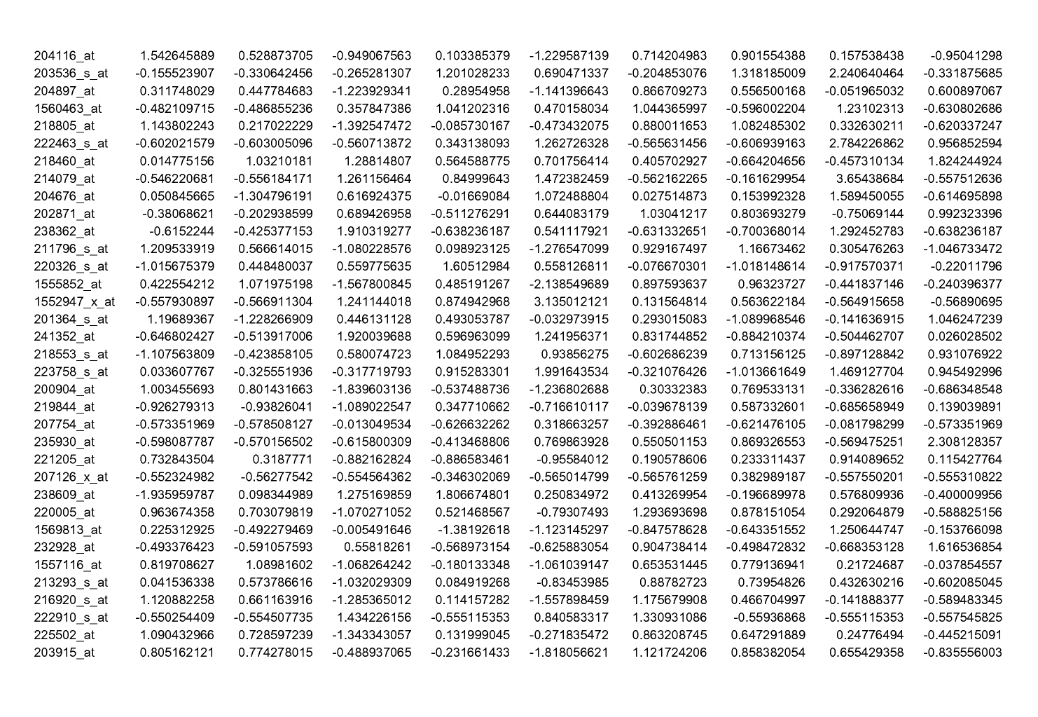

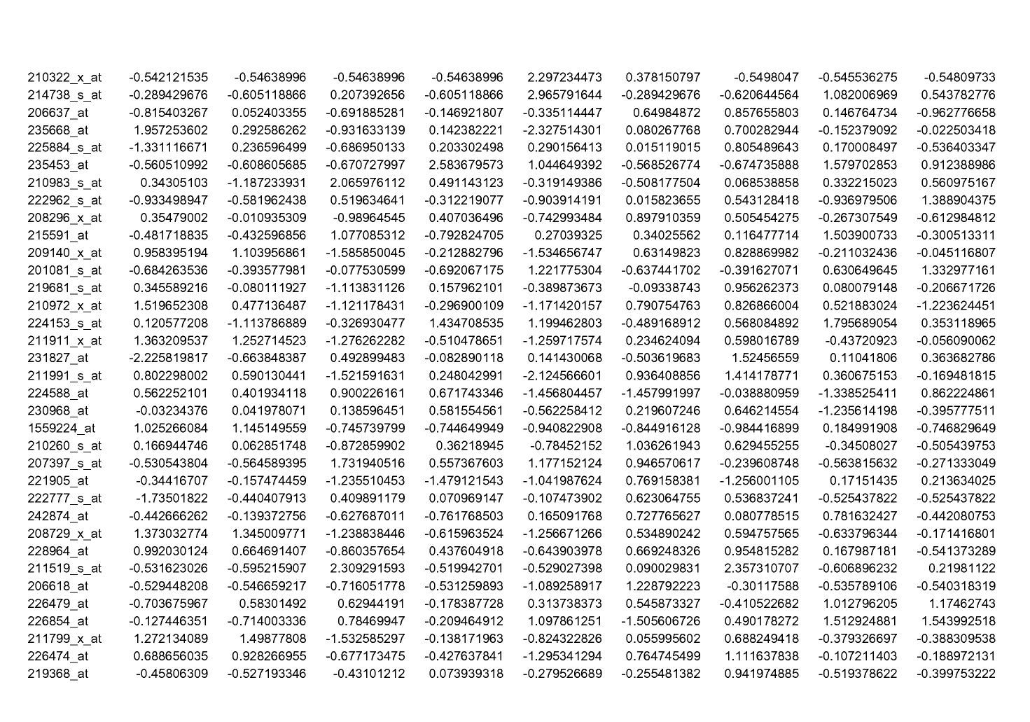

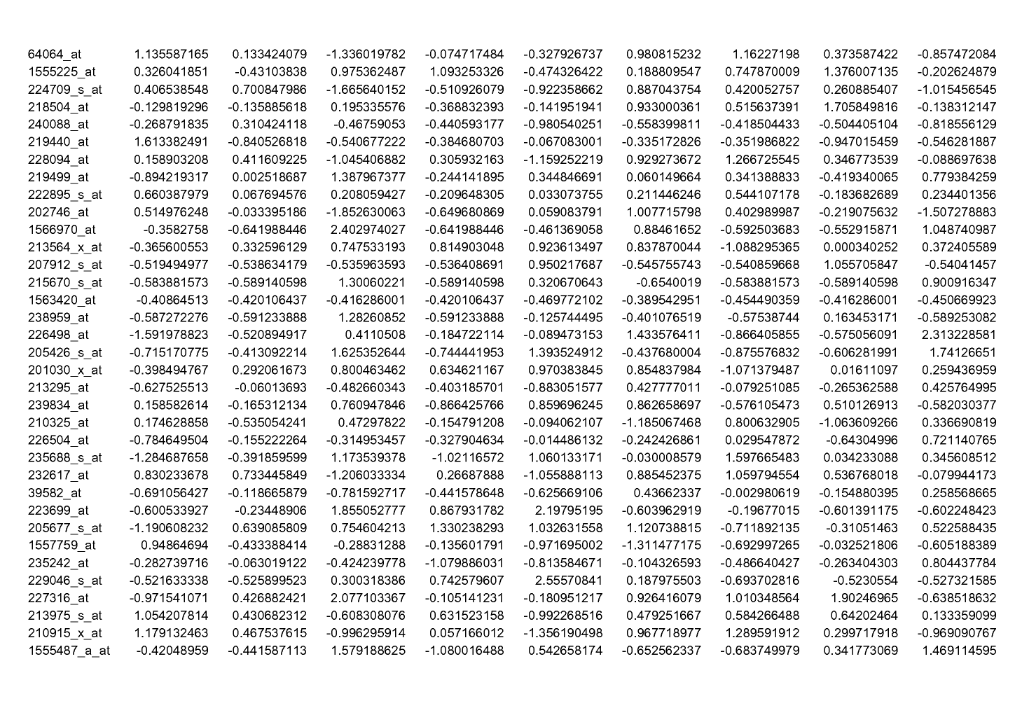

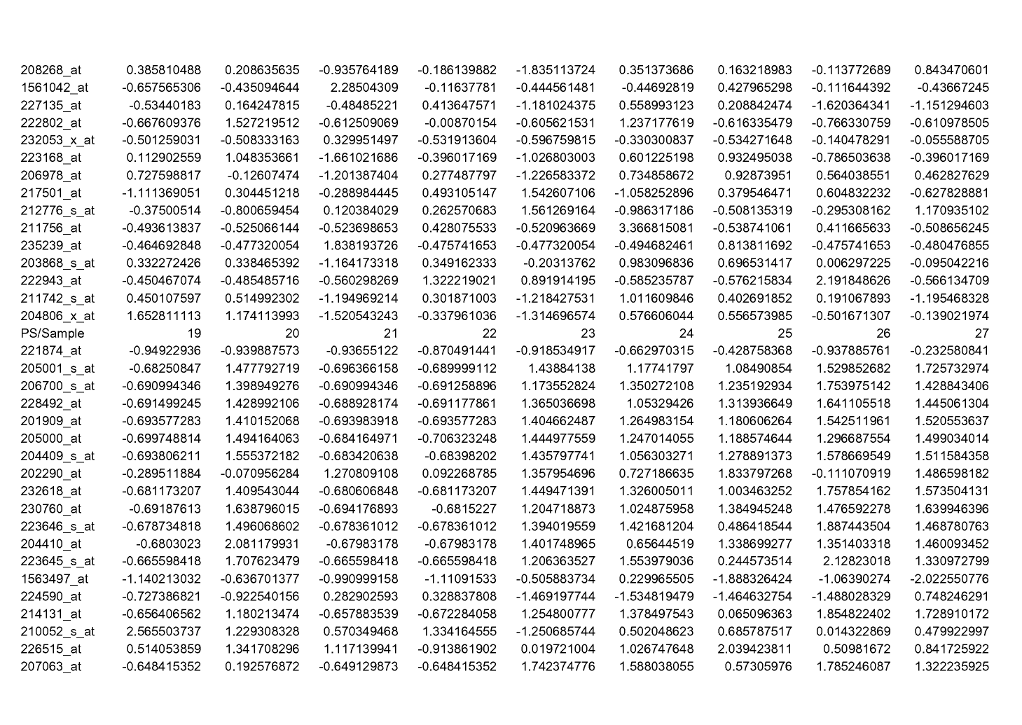

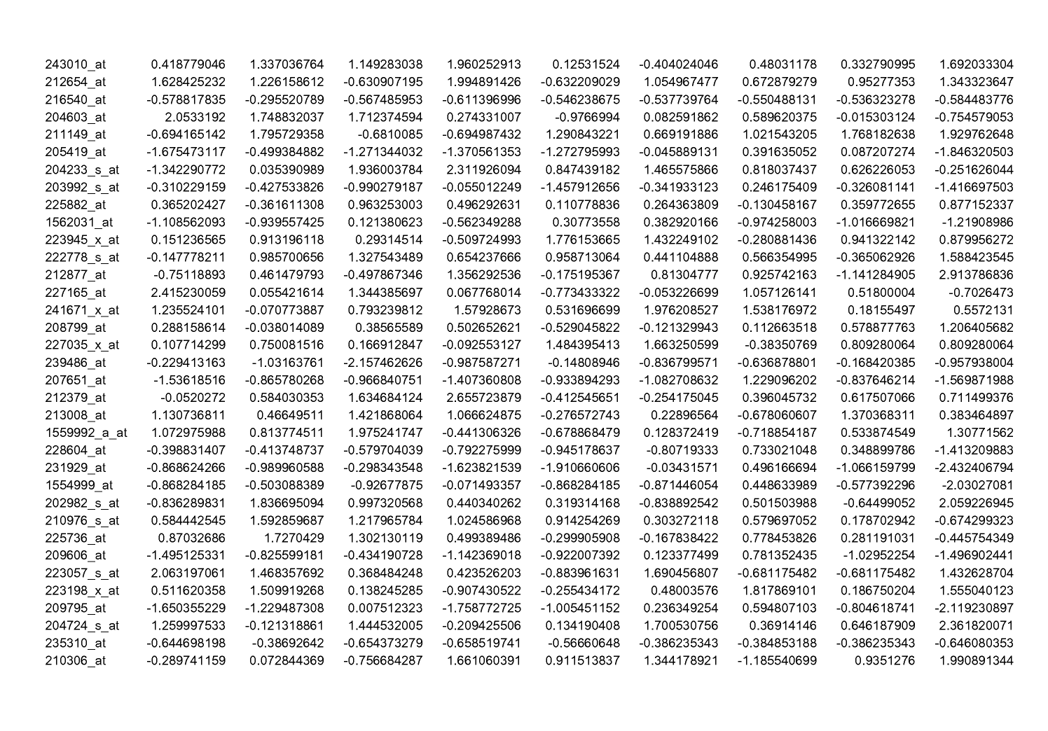

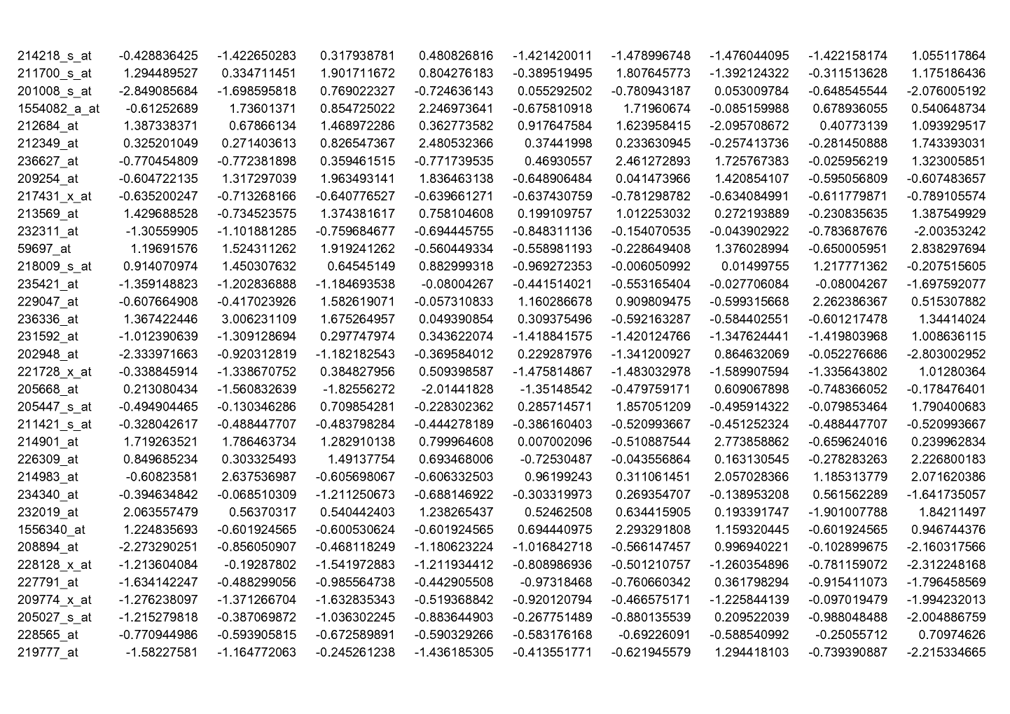

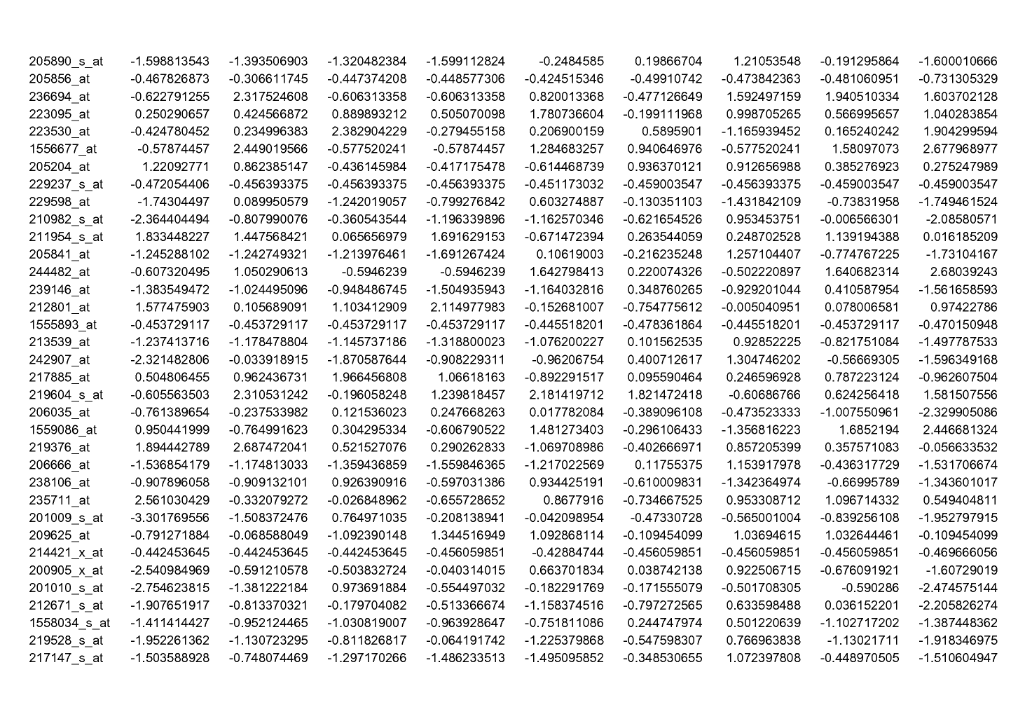

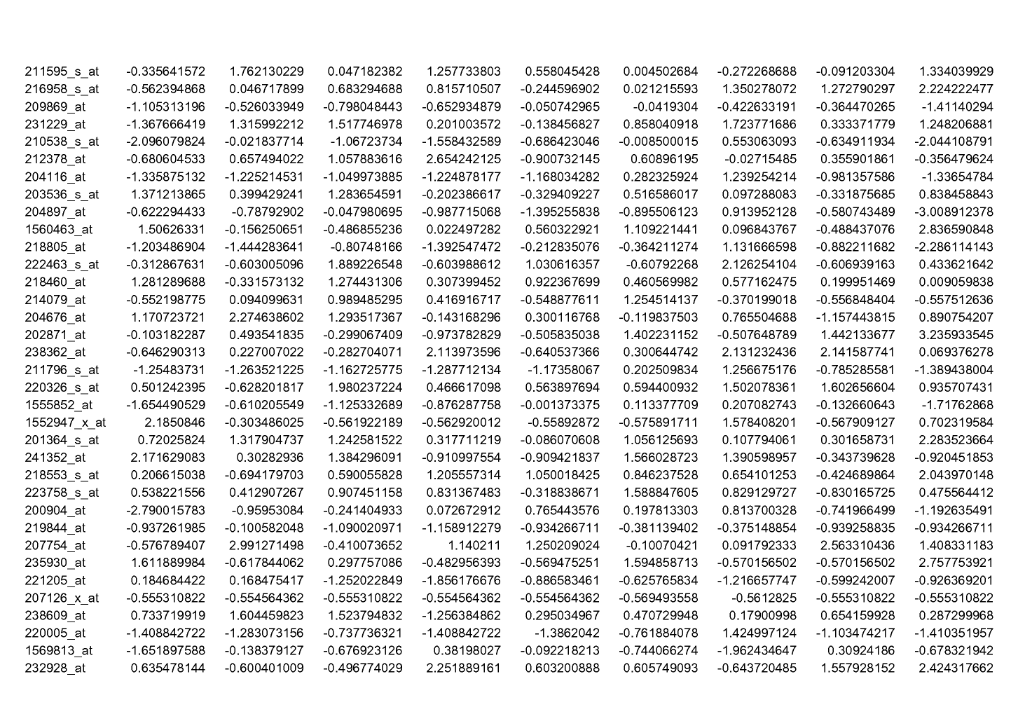

library (class)
knn (t (train), t (test), k=5)
where
train is an object (class matrix) of 250 rows and 27 columns to be built from Appendix B table (FIG. 10)
test is an object of class matrix containing the 250 feature probe sets of table 2 as rows and expression data (GCRMA processed and Z-score normalized) of the further samples to predict as columns.

Probe set order of test has to be identical to train, and Z-score calculation has to be computed for each gene using the medians (MeTr) and standard deviations (SdTr) depicted in the table 3:

### FIG. 2 Legend.

X-axis, number of probe sets used as features of the predictive model, in increasing order and log scale. Y-axis, 5NN predictive model performances, Se (empty circles) and PPV (solid dots) measures. The horizontal dash-line is the basal vaccine treatment efficacy not taking into account predictive model based patient selection. The vertical dash-line indicates the classification optimum, i.e. the smallest feature list size for which the Se is maximum associated to the maximum PPV given this highest Se.

Table 2: Affymetrix probe sets (250) used as 5NN model features for predictions of clinical outcome trained on second data stratification.
Table 3: Median (MeTr) and standard deviation values to be used for each probe set in the Z-score gene normalization process of additional samples to predict.

### Example 4

The list of genes in Table 4 was generated from samples in the MAGE 008 clinical trial which were prepared and analyzed as above. Thirty three samples from the AS15 arm and responders only from the AS02B arm were used. The data from these samples was analyzed using the Pearson Correlation Coefficient, which identifies genes based on the strength of the correlation between gene expression and clinical outcome. Suitable methodology is described in, for example Van't Veer 2002.

The selection leads to gene list 4. There was no evidence of a gender bias in this list generated.

In order to be able to classify samples prospectively using the above gene list, one needs to know the value that each ProbeSetld has in the metagene (as described by Van't Veer 2002). The values are provided in Table 7.

### Example 5

The list of genes in Table 5 was generated from samples in the mage 008 clinical trial, which were prepared and analyzed as above. Thirty three samples from the AS15 arm and responders only from the AS02B arm were used. The data from these samples was analyzed using Baldi statistical analysis (a variation of the classical T-test).

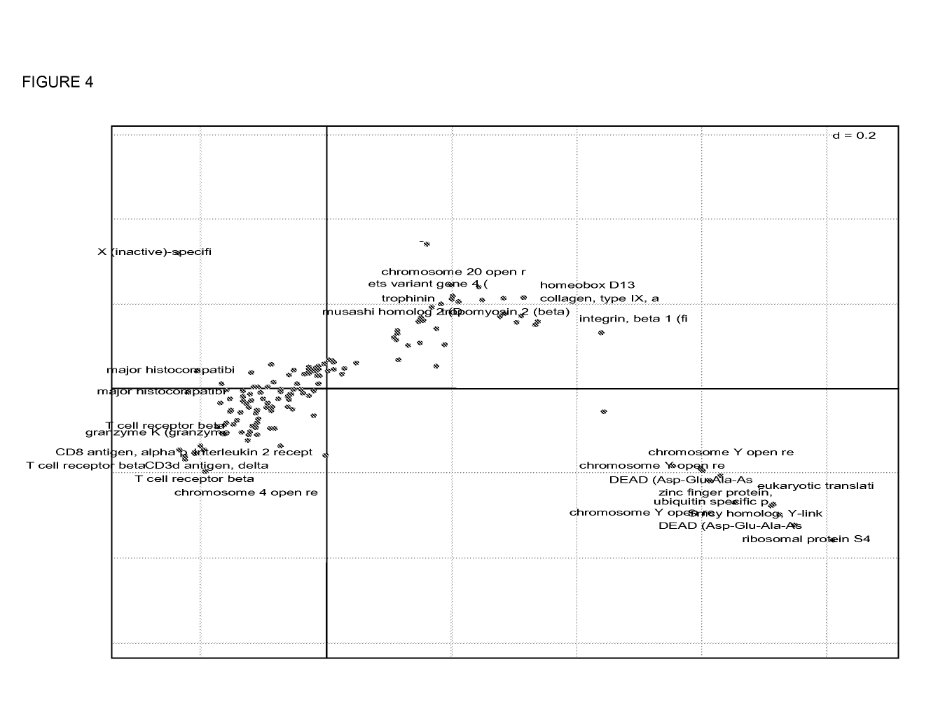

Multivariate analysis (Correspondance analysis) on the Baldi generated list showed clearly in FIG. 4, that gender specific genes are located in the upper-left and bottom-right quadrants. T-cell genes and other immune genes are found in the bottom-left and upper-right quadrants

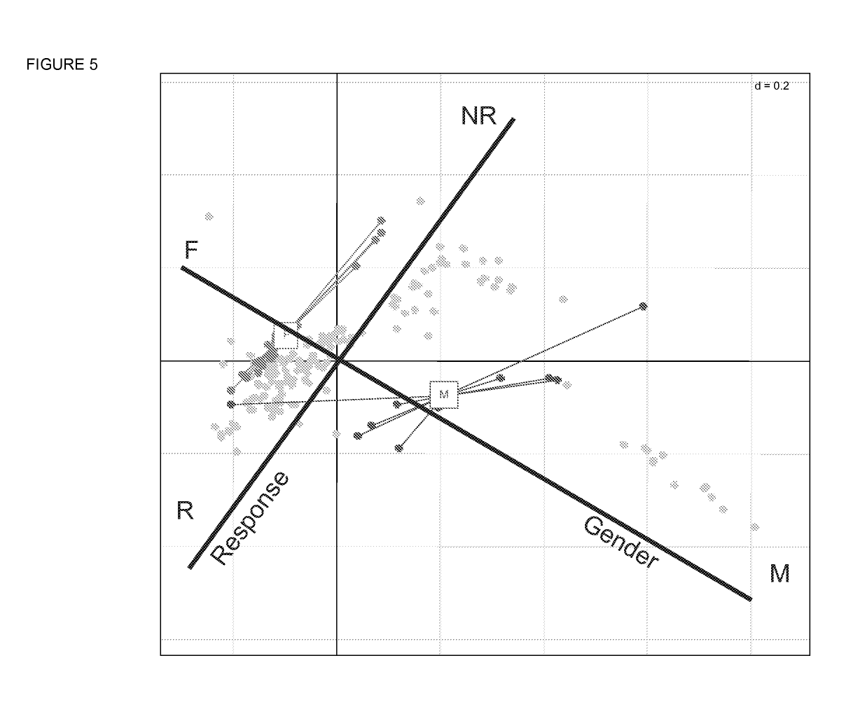

New axes that correlate with the segregation of the samples and genes by gender and response can be drawn as shown in FIG. 5

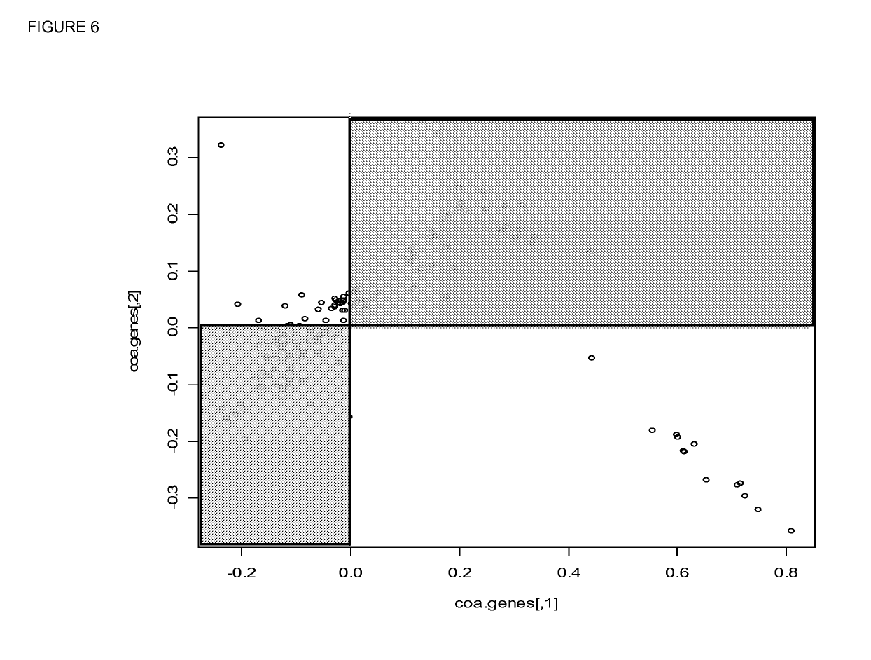

The genes that are of interest for classification of the patients are those located close to the Response axis. These genes are selected by keeping the genes that are in the lower left and upper right quadrants see FIG. 6. This eliminates all the genes that are strongly correlated with gender.

The selection leads to the gene list in Table 5, Multivariate Baldi gene list

The value of each ProbeSetld in the metagene for Table 5 is provided in Table 8.

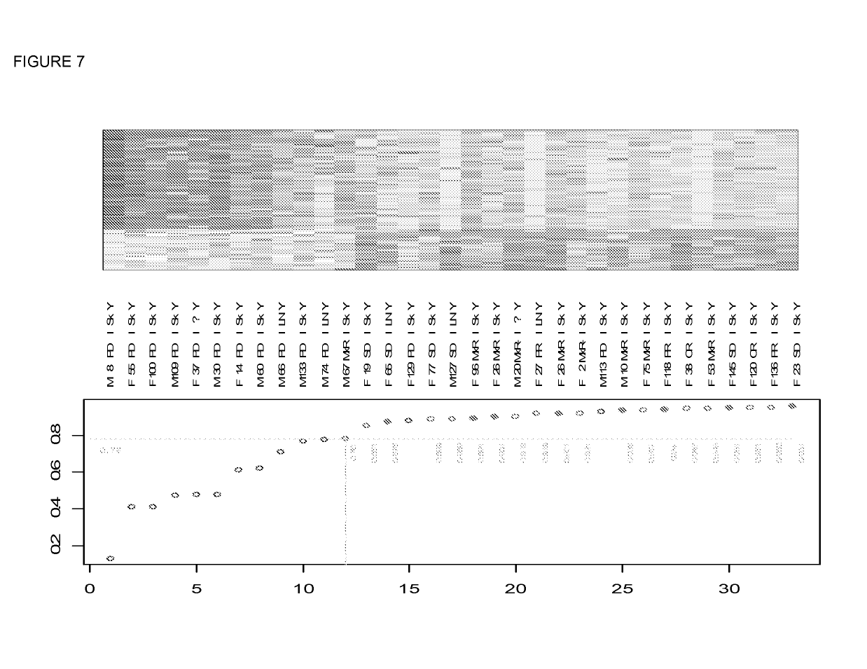

FIG. 7 shows the training set for Table 5 represented as an index. The indexing representation tool is likely to be useful for physicians because as time to treatment failure is a continuous variable and the results are with a range of very high time to treatment failure or a very lower time to treatment failure (rather than a simply binary output of responder or non-responder) then physicians can chose to treat those below the cutoff line and hence characterized as non-responder but nevertheless near to it. Thus presenting the data in this way provides the clinician with more information and allows more flexibility in the approach taken.

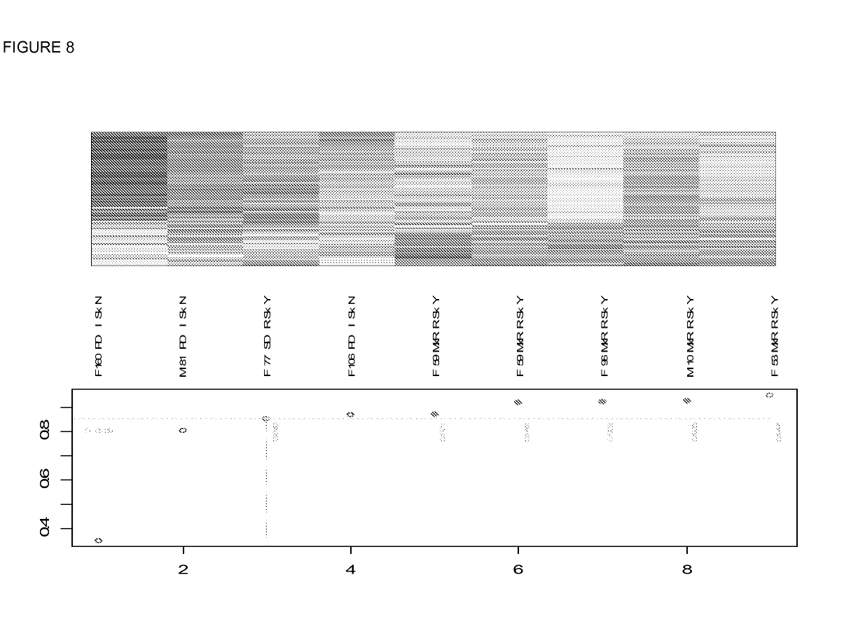

FIG. 8 shows the gene list of Table 5 used to predict on a small number of samples. Based on the clinical results only one patient was wrongly categorized as a responder (F106PDISKN).

### Example 6

Supervised Principal Components Analysis (SPCA) was used to analyze the samples. The main reason for using such an approach is that it allows gene to be selected by using a continuous variables of clinical outcome such as TTTF. Continuous variables are more information rich than binary variables.

The SPCA method operates in two steps:

1) A survival analysis is performed for each gene on the array. A Cox statistic is calculated for each gene. This statistic measures the strength of the association between the expression of a gene and the survival data. Genes are ranked by decreasing Cox score and the top-of-the-list genes are selected.
2) A Principal Component Analysis is performed using the genes identified in step 1. As expected, the samples will be distributed according to clinical outcome. A threshold can be set to define Responders and Non-Responders. These thresholds can be used to classify new patients (test samples).

It is expected that interference of gender with this analysis is minimal since TTTF and gender should not be correlated at the gene expression level.

The analysis yielded the list of genes in Table 6.

### Tables

TABLE 1
Affymetrix probe sets (18) used as 5NN model features for predictions of clinical
outcome trained on first data stratification.

| Probe Set ID | Symbol | Gene Name |
|---|---|---|
| 229022_at | ZFX | zinc finger protein, X-linked |
| 215666_at | HLA-DRB4 | major histocompatibility complex, class II, DR beta 4 |
| 1562031_at | JAK2 | Janus kinase 2 (a protein tyrosine kinase) |
| 233976_at | NA | NA |
| 212776_s_at | OBSL1 | obscurin-like 1 |
| 223095_at | MARVELD1 | MARVEL domain containing 1 |
| 219551_at | EAF2 | ELL associated factor 2 |
| 244008_at | NA | NA |
| 202290_at | PDAP1 | PDGFA associated protein 1 |
| 205518_s_at | CMAH | cytidine monophosphate-N-acetylneuraminic acid hydroxylase (CMP-N- |
| | | acetylneuraminate monooxygenase) |
| 227927_at | NA | NA |
| 228128_x_at | PAPPA | pregnancy-associated plasma protein A, pappalysin 1 |
| 220301_at | CCDC102B | coiled-coil domain containing 102B |
| 203992_s_at | UTX | ubiquitously transcribed tetratricopeptide repeat, X chromosome |
| 206618_at | IL18R1 | interleukin 18 receptor 1 |
| 228812_at | NA | NA |
| 1563497_at | USP25 | ubiquitin specific peptidase 25 |
| 212684_at | ZNF3 | zinc finger protein 3 |

TABLE 2
Affymetrix probe sets (250) used as 5NN model features for predictions of
clinical outcome trained on second data stratification.

| Probe Set ID | Symbol | Gene Name |
|---|---|---|
| 229022_at | ZFX | zinc finger protein, X-linked |
| 215666_at | HLA-DRB4 | major histocompatibility complex, class II, DR beta 4 |
| 1562031_at | JAK2 | Janus kinase 2 (a protein tyrosine kinase) |
| 233976_at | NA | NA |
| 212776_s_at | OBSL1 | obscurin-like 1 |
| 223095_at | MARVELD1 | MARVEL domain containing 1 |
| 219551_at | EAF2 | ELL associated factor 2 |
| 244008_at | NA | NA |
| 202290_at | PDAP1 | PDGFA associated protein 1 |
| 205518_s_at | CMAH | cytidine monophosphate-N-acetylneuraminic acid hydroxylase (CMP-N- |
| | | acetylneuraminate monooxygenase) |
| 227927_at | NA | NA |
| 228128_x_at | PAPPA | pregnancy-associated plasma protein A, pappalysin 1 |
| 220301_at | CCDC102B | coiled-coil domain containing 102B |
| 203992_s_at | UTX | ubiquitously transcribed tetratricopeptide repeat, X chromosome |
| 206618_at | IL18R1 | interleukin 18 receptor 1 |
| 228812_at | NA | NA |
| 1563497_at | USP25 | ubiquitin specific peptidase 25 |
| 212684_at | ZNF3 | zinc finger protein 3 |
| 224590_at | XIST | X (inactive)-specific transcript |
| 201364_s_at | OAZ2 | ornithine decarboxylase antizyme 2 |
| 229094_at | LOC401431 | hypothetical gene LOC401431 |
| 238106_at | COBLL1 | COBL-like 1 |
| 218553_s_at | KCTD15 | potassium channel tetramerisation domain containing 15 |
| 225150_s_at | RTKN | rhotekin |
| 205841_at | JAK2 | Janus kinase 2 (a protein tyrosine kinase) |
| 229598_at | COBLL1 | COBL-like 1 |
| 224941_at | PAPPA | pregnancy-associated plasma protein A, pappalysin 1 |
| 205027_s_at | MAP3K8 | mitogen-activated protein kinase kinase kinase 8 |
| 201870_at | TOMM34 | translocase of outer mitochondrial membrane 34 |
| 221905_at | CYLD | cylindromatosis (turban tumour syndrome) |
| 228017_s_at | C20orf58 | chromosome 20 open reading frame 58 |
| 205419_at | EBI2 | Epstein-Barr virus induced gene 2 (lymphocyte-specific G protein- |
| | | coupled receptor) |
| 205001_s_at | DDX3Y | DEAD (Asp-Glu-Ala-Asp) box polypeptide 3, Y-linked |
| 226152_at | TTC7B | tetratricopeptide repeat domain 7B |
| 206700_s_at | JARID1D | jumonji, AT rich interactive domain 1D |
| 228492_at | USP9Y | ubiquitin specific peptidase 9, Y-linked (fat facets-like, Drosophila) |
| 206118_at | STAT4 | signal transducer and activator of transcription 4 |
| 201909_at | RPS4Y1 | ribosomal protein S4, Y-linked 1 |
| 204724_s_at | COL9A3 | collagen, type IX, alpha 3 |
| 244370_at | KIAA2022 | KIAA2022 |
| 204409_s_at | EIF1AY | eukaryotic translation initiation factor 1A, Y-linked |
| 219901_at | FGD6 | FYVE, RhoGEF and PH domain containing 6 |
| 230760_at | ZFY | zinc finger protein, Y-linked |
| 205000_at | DDX3Y | DEAD (Asp-Glu-Ala-Asp) box polypeptide 3, Y-linked |
| 226854_at | GTPBP5 | GTP binding protein 5 (putative) |
| 232618_at | CYorf15A | chromosome Y open reading frame 15A |
| 231229_at | HILS1 | histone linker H1 domain, spermatid-specific 1 |
| 227073_at | MAP3K2 | mitogen-activated protein kinase kinase kinase 2 |
| 205447_s_at | MAP3K12 | mitogen-activated protein kinase kinase kinase 12 |
| 223646_s_at | CYorf15B | chromosome Y open reading frame 15B |
| 218838_s_at | TTC31 | tetratricopeptide repeat domain 31 |
| 206637_at | P2RY14 | purinergic receptor P2Y, G-protein coupled, 14 |
| 204410_at | EIF1AY | eukaryotic translation initiation factor 1A, Y-linked |
| 39582_at | CYLD | cylindromatosis (turban tumour syndrome) |
| 210258_at | RGS13 | regulator of G-protein signaling 13 |
| 212801_at | CIT | citron (rho-interacting, serine/threonine kinase 21) |
| 242469_at | LOC120376 | hypothetical protein LOC120376 |
| 227119_at | CNOT6L | CCR4-NOT transcription complex, subunit 6-like |
| 239486_at | NA | NA |
| 222778_s_at | WHSC1 | Wolf-Hirschhorn syndrome candidate 1 |
| 239146_at | CLDND1 | claudin domain containing 1 |
| 229308_at | NA | NA |
| 216540_at | TRA@ | T cell receptor alpha locus |
| 202580_x_at | FOXM1 | forkhead box M1 |
| 230164_at | ZNF621 | zinc finger protein 621 |
| 203756_at | ARHGEF17 | Rho guanine nucleotide exchange factor (GEF) 17 |
| 227265_at | FGL2 | fibrinogen-like 2 |
| 226207_at | FLJ39378 | hypothetical protein FLJ39378 |
| 212877_at | KLC1 | kinesin light chain 1 |
| 211603_s_at | ETV4 | ets variant gene 4 (E1A enhancer binding protein, E1AF) |
| 213295_at | CYLD | cylindromatosis (turban tumour syndrome) |
| 223645_s_at | CYorf15B | chromosome Y open reading frame 15B |
| 235092_at | KCTD1 | potassium channel tetramerisation domain containing 1 |
| 224940_s_at | PAPPA | pregnancy-associated plasma protein A, pappalysin 1 |
| 223407_at | C16orf48 | chromosome 16 open reading frame 48 |
| 211149_at | UTY | ubiquitously transcribed tetratricopeptide repeat gene, Y-linked |
| 1561226_at | NA | NA |
| 243010_at | MSI2 | musashi homolog 2 (Drosophila) |
| 225882_at | SLC35B4 | solute carrier family 35, member B4 |
| 230968_at | NA | NA |
| 216958_s_at | IVD | isovaleryl Coenzyme A dehydrogenase |
| 226972_s_at | CCDC136 | coiled-coil domain containing 136 |
| 244393_x_at | AKR1C2 | aldo-keto reductase family 1, member C2 (dihydrodiol dehydrogenase 2; |
| | | bile acid binding protein; 3-alpha hydroxysteroid dehydrogenase, type III) |
| 219844_at | C10orf118 | chromosome 10 open reading frame 118 |
| 222859_s_at | DAPP1 | dual adaptor of phosphotyrosine and 3-phosphoinositides |
| 214131_at | CYorf15B | chromosome Y open reading frame 15B |
| 205105_at | MAN2A1 | mannosidase, alpha, class 2A, member 1 |
| 204434_at | SPATA2 | spermatogenesis associated 2 |
| 227263_at | C8orf58 | chromosome 8 open reading frame 58 |
| 228071_at | GIMAP7 | GTPase, IMAP family member 7 |
| 217778_at | SLC39A1 | solute carrier family 39 (zinc transporter), member 1 |
| 202948_at | IL1R1 | interleukin 1 receptor, type I |
| 1554999_at | RASGEF1B | RasGEF domain family, member 1B |
| 201115_at | POLD2 | polymerase (DNA directed), delta 2, regulatory subunit 50 kDa |
| 204947_at | E2F1 | E2F transcription factor 1 |
| 244272_s_at | TC2N | tandem C2 domains, nuclear |
| 239834_at | KCTD1 | potassium channel tetramerisation domain containing 1 |
| 213618_at | CENTD1 | centaurin, delta 1 |
| 213547_at | CAND2 | cullin-associated and neddylation-dissociated 2 (putative) |
| 210052_s_at | TPX2 | TPX2, microtubule-associated, homolog (Xenopus laevis) |
| 207063_at | CYorf14 | chromosome Y open reading frame 14 |
| 210976_s_at | PFKM | phosphofructokinase, muscle |
| 221759_at | G6PC3 | glucose 6 phosphatase, catalytic, 3 |
| 231715_s_at | PYCR2 | pyrroline-5-carboxylate reductase family, member 2 |
| 235310_at | GCET2 | germinal center expressed transcript 2 |
| 1568752_s_at | RGS13 | regulator of G-protein signaling 13 |
| 201087_at | PXN | paxillin |
| 226043_at | GPSM1 | G-protein signaling modulator 1 (AGS3-like, C. elegans) |
| 212775_at | OBSL1 | obscurin-like 1 |
| 209262_s_at | NR2F6 | nuclear receptor subfamily 2, group F, member 6 |
| 212654_at | TPM2 | tropomyosin 2 (beta) |
| 210017_at | MALT1 | mucosa associated lymphoid tissue lymphoma translocation gene 1 |
| 231094_s_at | MTHFD1L | methylenetetrahydrofolate dehydrogenase (NADP+ dependent) 1-like |
| 229623_at | FLJ12993 | hypothetical LOC441027 |
| 229575_at | NA | NA |
| 216438_s_at | TMSB4X | thymosin, beta 4, X-linked |
| 209728_at | HLA-DRB4 | major histocompatibility complex, class II, DR beta 4 |
| 205204_at | NMB | neuromedin B |
| 227908_at | TBC1D24 | TBC1 domain family, member 24 |
| 235885_at | P2RY12 | purinergic receptor P2Y, G-protein coupled, 12 |
| 213569_at | NA | NA |
| 212134_at | PHLDB1 | pleckstrin homology-like domain, family B, member 1 |
| 1562249_at | LOC285965 | hypothetical protein LOC285965 |
| 218786_at | NT5DC3 | 5′-nucleotidase domain containing 3 |
| 208799_at | PSMB5 | proteasome (prosome, macropain) subunit, beta type, 5 |
| 211676_s_at | IFNGR1 | interferon gamma receptor 1 |
| 221789_x_at | RHOT2 | ras homolog gene family, member T2 |
| 231929_at | IKZF2 | IKAROS family zinc finger 2 (Helios) |
| 226470_at | GGTL3 | gamma-glutamyltransferase-like 3 |
| 209795_at | CD69 | CD69 molecule |
| 241671_x_at | FLJ22536 | hypothetical locus LOC401237 |
| 224859_at | CD276 | CD276 molecule |
| 220005_at | P2RY13 | purinergic receptor P2Y, G-protein coupled, 13 |
| 202578_s_at | DDX19A | DEAD (Asp-Glu-Ala-As) box polypeptide 19A |
| 223284_at | NAT14 | N-acetyltransferase 14 |
| 231890_at | NA | NA |
| 224709_s_at | CDC42SE2 | CDC42 small effector 2 |
| 204078_at | SC65 | synaptonemal complex protein SC65 |
| 209606_at | PSCDBP | pleckstrin homology, Sec7 and coiled-coil domains, binding protein |
| 205668_at | LY75 | lymphocyte antigen 75 |
| 208688_x_at | EIF3B | eukaryotic translation initiation factor 3, subunit B |
| 223198_x_at | COMMD5 | COMM domain containing 5 |
| 220168_at | CASC1 | cancer susceptibility candidate 1 |
| 229026_at | CDC42SE2 | CDC42 small effector 2 |
| 243170_at | LOC339745 | hypothetical protein LOC339745 |
| 1556209_at | CLEC2B | C-type lectin domain family 2, member B |
| 52164_at | C11orf24 | chromosome 11 open reading frame 24 |
| 231592_at | XIST | X (inactive)-specific transcript |
| 209078_s_at | TXN2 | thioredoxin 2 |
| 239726_at | ANK3 | ankyrin 3, node of Ranvier (ankyrin G) |
| 213587_s_at | ATP6V0E2 | ATPase, H+ transporting V0 subunit e2 |
| 219343_at | CDC37L1 | cell division cycle 37 homolog (S. cerevisiae)-like 1 |
| 218723_s_at | C13orf15 | chromosome 13 open reading frame 15 |
| 204233_s_at | CHKA | choline kinase alpha |
| 220241_at | TMCO3 | transmembrane and coiled-coil domains 3 |
| 205856_at | SLC14A1 | solute carrier family 14 (urea transporter), member 1 (Kidd blood group) |
| 201981_at | PAPPA | pregnancy-associated plasma protein A, pappalysin 1 |
| 213196_at | ZNF629 | zinc finger protein 629 |
| 225531_at | CABLES1 | Cdk5 and Abl enzyme substrate 1 |
| 228094_at | AMICA1 | adhesion molecule, interacts with CXADR antigen 1 |
| 1565544_at | RNF141 | ring finger protein 141 |
| 207651_at | GPR171 | G protein-coupled receptor 171 |
| 227929_at | NA | NA |
| 203679_at | TMED1 | transmembrane emp24 protein transport domain containing 1 |
| 214901_at | ZNF8 | zinc finger protein 8 |
| 201008_s_at | TXNIP | thioredoxin interacting protein |
| 203642_s_at | COBLL1 | COBL-like 1 |
| 222802_at | EDN1 | endothelin 1 |
| 212379_at | GART | phosphoribosylglycinamide formyltransferase, |
| | | phosphoribosylglycinamide synthetase, phosphoribosylaminoimidazole |
| | | synthetase |
| 226971_at | CCDC136 | coiled-coil domain containing 136 |
| 218956_s_at | PTCD1 | pentatricopeptide repeat domain 1 |
| 227135_at | ASAHL | N-acylsphingosine amidohydrolase (acid ceramidase)-like |
| 229047_at | PLEKHB1 | pleckstrin homology domain containing, family B (evectins) member 1 |
| 209277_at | TFPI2 | tissue factor pathway inhibitor 2 |
| 209129_at | TRIP6 | thyroid hormone receptor interactor 6 |
| 228565_at | KIAA1804 | mixed lineage kinase 4 |
| 239499_at | DNAH2 | dynein, axonemal, heavy chain 2 |
| 228054_at | TMEM44 | transmembrane protein 44 |
| 213551_x_at | CISD3 | CDGSH iron sulfur domain 3 |
| 242765_at | MOBP | myelin-associated oligodendrocyte basic protein |
| 215314_at | ANK3 | ankyrin 3, node of Ranvier (ankyrin G) |
| 222462_s_at | BACE1 | beta-site APP-cleaving enzyme 1 |
| 213077_at | YTHDC2 | YTH domain containing 2 |
| 209150_s_at | TM9SF1 | transmembrane 9 superfamily member 1 |
| 238693_at | PHC3 | polyhomeotic homolog 3 (Drosophila) |
| 225475_at | MIER1 | mesoderm induction early response 1 homolog (Xenopus laevis) |
| 222520_s_at | IFT57 | intraflagellar transport 57 homolog (Chlamydomonas) |
| 210260_s_at | TNFAIP8 | tumour necrosis factor, alpha-induced protein 8 |
| 208894_at | HLA-DRA | major histocompatibility complex, class II, DR alpha |
| 214218_s_at | XIST | X (inactive)-specific transcript |
| 208498_s_at | AMY1A | amylase, alpha 1A (salivary) |
| 231084_at | C10orf79 | chromosome 10 open reading frame 79 |
| 232114_at | MED12L | mediator complex subunit 12-like |
| 204897_at | PTGER4 | prostaglandin E receptor 4 (subtype EP4) |
| 221874_at | KIAA1324 | KIAA1324 |
| 221728_x_at | XIST | X (inactive)-specific transcript |
| 221205_at | NA | NA |
| 205987_at | CD1C | CD1c molecule |
| 200924_s_at | SLC3A2 | solute carrier family 3 (activators of dibasic and neutral amino acid |
| | | transport), member 2 |
| 201010_s_at | TXNIP | thioredoxin interacting protein |
| 209254_at | KIAA0265 | KIAA0265 protein |
| 244335_at | NA | NA |
| 203071_at | SEMA3B | sema domain, immunoglobulin domain (Ig), short basic domain, secreted, |
| | | (semaphorin) 3B |
| 219368_at | NAP1L2 | nucleosome assembly protein 1-like 2 |
| 35685_at | RING1 | ring finger protein 1 |
| 1558111_at | MBNL1 | muscleblind-like (Drosophila) |
| 211421_s_at | RET | ret proto-oncogene |
| 235048_at | KIAA0888 | KIAA0888 protein |
| 226538_at | MAN2A1 | mannosidase, alpha, class 2A, member 1 |
| 240013_at | NA | NA |
| 226118_at | CENPO | centromere protein O |
| 204778_x_at | HOXB7 | homeobox B7 |
| 210306_at | L3MBTL | l(3)mbt-like (Drosophila) |
| 203967_at | CDC6 | cell division cycle 6 homolog (S. cerevisiae) |
| 207714_s_at | SERPINH1 | serpin peptidase inhibitor, clade H (heat shock protein 47), member 1, |
| | | (collagen binding protein 1) |
| 236707_at | DAPP1 | dual adaptor of phosphotyrosine and 3-phosphoinositides |
| 225736_at | FBXO22 | F-box protein 22 |
| 222858_s_at | DAPP1 | dual adaptor of phosphotyrosine and 3-phosphoinositides |
| 212349_at | POFUT1 | protein O-fucosyltransferase 1 |
| 201938_at | CDK2AP1 | CDK2-associated protein 1 |
| 203577_at | GTF2H4 | general transcription factor IIH, polypeptide 4, 52 kDa |
| 223571_at | C1QTNF6 | C1q and tumour necrosis factor related protein 6 |
| 202727_s_at | IFNGR1 | interferon gamma receptor 1 |
| 227165_at | C13orf3 | chromosome 13 open reading frame 3 |
| 230330_at | NA | NA |
| 225230_at | TMEM77 | transmembrane protein 77 |
| 205997_at | ADAM28 | ADAM metallopeptidase domain 28 |
| 204921_at | GAS8 | growth arrest-specific 8 |
| 242874_at | C14orf161 | chromosome 14 open reading frame 161 |
| 232204_at | EBF1 | early B-cell factor 1 |
| 203793_x_at | PCGF2 | polycomb group ring finger 2 |
| 241956_at | PCGF5 | polycomb group ring finger 5 |
| 1556341_s_at | MAPK12 | mitogen-activated protein kinase 12 |
| 236694_at | CYorf15A | chromosome Y open reading frame 15A |
| 205421_at | SLC22A3 | solute carrier family 22 (extraneuronal monoamine transporter), member 3 |
| 215359_x_at | ZNF44 | zinc finger protein 44 |
| 235569_at | VPS37D | vacuolar protein sorting 37 homolog D (S. cerevisiae) |
| 1553555_at | TAS2R38 | taste receptor, type 2, member 38 |
| 220933_s_at | ZCCHC6 | zinc finger, CCHC domain containing 6 |
| 224588_at | XIST | X (inactive)-specific transcript |
| 214983_at | TTTY15 | testis-specific transcript, Y-linked 15 |
| 226042_at | EDC3 | enhancer of mRNA decapping 3 homolog (S. cerevisiae) |
| 222654_at | IMPAD1 | inositol monophosphatase domain containing 1 |
| 230521_at | C9orf100 | chromosome 9 open reading frame 100 |
| 217147_s_at | TRAT1 | T cell receptor associated transmembrane adaptor 1 |
| 212671_s_at | HLA-DQA1 | major histocompatibility complex, class II, DQ alpha 1 |
| 226515_at | CCDC127 | coiled-coil domain containing 127 |
| 228604_at | NA | NA |
| 208296_x_at | TNFAIP8 | tumour necrosis factor, alpha-induced protein 8 |
| 1557458_s_at | SHB | Src homology 2 domain containing adaptor protein B |

TABLE 3
Median (MeTr) and standard deviation values to be used for each probe set in
the Z-score gene normalization process of additional samples to predict.

| PS | MeTr | SdTr | PS | MeTr | SdTr |
|---|---|---|---|---|---|
| 221874_at | 3.735555556 | 1.498642599 | 227035_x_at | 8.078481481 | 0.7939384 |
| 205001_s_at | 4.250296296 | 2.669998067 | 239486_at | 7.085814815 | 1.18046764 |
| 206700_s_at | 4.832962963 | 3.780006273 | 207651_at | 7.212777778 | 2.7013526 |
| 228492_at | 4.42062963 | 3.11154299 | 212379_at | 5.208407407 | 0.776659279 |
| 201909_at | 5.660296296 | 4.918408348 | 213008_at | 7.903148148 | 0.951460889 |
| 205000_at | 5.191740741 | 4.106817592 | 1559992_a_at | 3.817111111 | 2.12575949 |
| 204409_s_at | 4.786777778 | 3.56263426 | 228604_at | 5.894888889 | 0.536288982 |
| 202290_at | 5.938296296 | 0.72292817 | 231929_at | 8.477703704 | 2.060388795 |
| 232618_at | 4.668444444 | 3.531325686 | 1554999_at | 3.834222222 | 0.632537401 |
| 230760_at | 4.131296296 | 2.607831398 | 202982_s_at | 3.71362963 | 0.768429325 |
| 223646_s_at | 4.036740741 | 2.675184316 | 210976_s_at | 9.03537037 | 0.842905148 |
| 204410_at | 3.710851852 | 2.1253079 | 225736_at | 7.191148148 | 0.870766934 |
| 223645_s_at | 3.871518519 | 2.479751265 | 209606_at | 5.603296296 | 2.250845616 |
| 1563497_at | 6.229481481 | 1.467691944 | 223057_s_at | 3.143407407 | 1.035573689 |
| 224590_at | 7.618444444 | 3.504936259 | 223198_x_at | 5.355444444 | 0.88650803 |
| 214131_at | 4.196703704 | 2.708235727 | 209795_at | 7.511222222 | 2.499596542 |
| 210052_s_at | 6.778111111 | 1.039518614 | 204724_s_at | 4.492851852 | 2.042978721 |
| 226515_at | 4.869037037 | 0.708024961 | 235310_at | 3.183888889 | 1.447016435 |
| 207063_at | 4.036962963 | 2.799074634 | 210306_at | 3.84737037 | 1.312793708 |
| 243010_at | 5.109148148 | 1.267617987 | 214218_s_at | 8.277851852 | 4.064141358 |
| 212654_at | 4.939259259 | 1.536294508 | 211700_s_at | 4.926333333 | 1.307593944 |
| 216540_at | 2.69362963 | 0.705972768 | 201008_s_at | 11.36133333 | 1.314222789 |
| 204603_at | 5.347666667 | 1.481179026 | 1554082_a_at | 3.402333333 | 0.426647936 |
| 211149_at | 4.00937037 | 2.432231567 | 212684_at | 8.63137037 | 0.845236933 |
| 205419_at | 6.541814815 | 2.066171507 | 212349_at | 7.416888889 | 0.873647585 |
| 204233_s_at | 4.973444444 | 1.428486648 | 236627_at | 3.457407407 | 1.556752445 |
| 203992_s_at | 5.392703704 | 0.630835942 | 209254_at | 2.963962963 | 0.724238352 |
| 225882_at | 6.582185185 | 1.289188625 | 217431_x_at | 2.931555556 | 0.896655123 |
| 1562031_at | 6.418111111 | 1.556170036 | 213569_at | 4.402592593 | 1.518797532 |
| 223945_x_at | 7.422703704 | 0.78219375 | 232311_at | 6.727333333 | 1.624796934 |
| 222778_s_at | 5.146555556 | 1.445108549 | 59697_at | 2.713740741 | 0.681133365 |
| 212877_at | 5.259888889 | 1.038206046 | 218009_s_at | 7.019037037 | 0.997693821 |
| 227165_at | 4.345666667 | 1.214929121 | 235421_at | 5.102703704 | 1.433031961 |
| 241671_x_at | 5.631851852 | 1.41085726 | 229047_at | 3.83237037 | 1.437256552 |
| 208799_at | 10.34044444 | 0.564118327 | 236336_at | 2.739814815 | 0.773122591 |
| 231592_at | 6.629851852 | 3.117227413 | 206035_at | 5.663518519 | 0.983095205 |
| 202948_at | 7.906666667 | 1.370910674 | 1559086_at | 3.837703704 | 1.049297377 |
| 221728_x_at | 9.065259259 | 4.294752277 | 219376_at | 3.87862963 | 1.158847543 |
| 205668_at | 8.345259259 | 1.805612715 | 206666_at | 6.727444444 | 2.914033424 |
| 205447_s_at | 4.681074074 | 0.990239751 | 238106_at | 4.396037037 | 1.618067425 |
| 211421_s_at | 2.538111111 | 0.430160912 | 235711_at | 6.971407407 | 0.76008179 |
| 214901_at | 4.256185185 | 1.116067893 | 201009_s_at | 11.53896296 | 1.210551765 |
| 226309_at | 3.79837037 | 1.248261811 | 209625_at | 5.637888889 | 0.464933604 |
| 214983_at | 3.198703704 | 1.57620398 | 214421_x_at | 2.250518519 | 0.073495877 |
| 234340_at | 4.440518519 | 0.76657833 | 200905_x_at | 10.67244444 | 1.201677492 |
| 232019_at | 6.387148148 | 1.074771056 | 201010_s_at | 12.12874074 | 1.117662863 |
| 1556340_at | 3.07162963 | 1.434780502 | 212671_s_at | 8.684185185 | 2.733300105 |
| 208894_at | 11.48414815 | 1.672970773 | 1558034_s_at | 6.453777778 | 2.795619559 |
| 228128_x_at | 6.448555556 | 1.796760233 | 219528_s_at | 6.061814815 | 1.975562744 |
| 227791_at | 6.946962963 | 1.453951128 | 217147_s_at | 6.370851852 | 2.708088479 |
| 209774_x_at | 6.540148148 | 2.083583114 | 211595_s_at | 6.282518519 | 0.773201356 |
| 205027_s_at | 4.596185185 | 1.13980761 | 216958_s_at | 4.95037037 | 1.019515652 |
| 228565_at | 2.713111111 | 0.559198281 | 209869_at | 4.65737037 | 1.702115181 |
| 219777_at | 8.813481481 | 1.396394654 | 231229_at | 6.128407407 | 1.87356169 |
| 205890_s_at | 8.542185185 | 3.341343466 | 210538_s_at | 7.208481481 | 2.174288131 |
| 205856_at | 2.911851852 | 0.831187506 | 212378_at | 8.916666667 | 0.576938072 |
| 236694_at | 3.213888889 | 1.517183938 | 204116_at | 6.18562963 | 2.973054544 |
| 223095_at | 10.61007407 | 1.130389481 | 203536_s_at | 6.497111111 | 0.810879263 |
| 223530_at | 4.503444444 | 1.032167187 | 204897_at | 9.082296296 | 1.756879441 |
| 1556677_at | 2.714703704 | 0.816774322 | 1560463_at | 5.278777778 | 0.632175142 |
| 205204_at | 4.921814815 | 1.05426814 | 218805_at | 10.71722222 | 1.565635834 |
| 229237_s_at | 2.458851852 | 0.383116542 | 222463_s_at | 2.867111111 | 1.01675942 |
| 229598_at | 6.073777778 | 1.870162752 | 218460_at | 7.073074074 | 0.874841917 |
| 210982_s_at | 12.02188889 | 1.658298696 | 214079_at | 3.101333333 | 1.505496519 |
| 211954_s_at | 9.853185185 | 0.606406441 | 204676_at | 5.989592593 | 0.814374383 |
| 205841_at | 6.449518519 | 1.181669138 | 202871_at | 2.781888889 | 0.551343557 |
| 244482_at | 3.238 | 1.417702855 | 238362_at | 2.938703704 | 0.869119794 |
| 239146_at | 6.817148148 | 1.762964171 | 211796_s_at | 6.711037037 | 3.224351879 |
| 212801_at | 6.47637037 | 0.866973389 | 220326_s_at | 4.435 | 1.212985985 |
| 1555893_at | 2.295259259 | 0.121789097 | 1555852_at | 6.557740741 | 1.995623839 |
| 213539_at | 7.035296296 | 3.206927679 | 1552947_x_at | 2.838148148 | 1.002181724 |
| 242907_at | 8.783481481 | 1.783119595 | 201364_s_at | 5.101703704 | 0.809843281 |
| 217885_at | 8.076074074 | 0.867512533 | 241352_at | 4.007444444 | 1.90389583 |
| 219604_s_at | 3.202333333 | 0.766778927 | 218553_s_at | 4.482592593 | 1.202271672 |
| 223758_s_at | 7.231962963 | 0.893752826 | 207397_s_at | 3.032666667 | 1.292384647 |
| 200904_at | 11.96414815 | 1.222626829 | 221905_at | 6.601333333 | 0.878449333 |
| 219844_at | 4.545740741 | 1.00157774 | 222777_s_at | 4.825740741 | 0.835000304 |
| 207754_at | 2.628592593 | 0.581828633 | 242874_at | 5.062037037 | 1.707916555 |
| 235930_at | 3.180925926 | 1.467888068 | 208729_x_at | 10.43914815 | 1.570138669 |
| 221205_at | 6.483666667 | 0.678635112 | 228964_at | 8.553814815 | 1.316678953 |
| 207126_x_at | 3.091925926 | 1.339656814 | 211519_s_at | 2.96462963 | 0.770526501 |
| 238609_at | 5.892 | 0.904977475 | 206618_at | 3.472481481 | 1.103944584 |
| 220005_at | 5.036444444 | 1.987762297 | 226479_at | 9.582481481 | 0.75387182 |
| 1569813_at | 5.990925926 | 0.71489052 | 226854_at | 5.276555556 | 0.804695897 |
| 232928_at | 3.541851852 | 1.177299573 | 211799_x_at | 7.847962963 | 1.447203603 |
| 1557116_at | 10.29211111 | 1.799284331 | 226474_at | 8.065851852 | 2.115930268 |
| 213293_s_at | 10.33585185 | 1.544386229 | 219368_at | 4.653 | 1.663526306 |
| 216920_s_at | 5.415592593 | 2.06213221 | 64064_at | 10.264 | 1.686352276 |
| 222910_s_at | 3.173592593 | 1.645770716 | 1555225_at | 6.714 | 1.085750184 |
| 225502_at | 6.708851852 | 1.857196371 | 224709_s_at | 12.63725926 | 0.66596573 |
| 203915_at | 9.095111111 | 3.626460823 | 218504_at | 4.864 | 0.824222617 |
| 210322_x_at | 2.881037037 | 1.171392383 | 240088_at | 2.848518519 | 0.407447342 |
| 214738_s_at | 2.354925926 | 0.193228029 | 219440_at | 3.258814815 | 1.070536711 |
| 206637_at | 9.558148148 | 1.886364956 | 228094_at | 6.580740741 | 1.958797838 |
| 235668_at | 4.768925926 | 0.885462197 | 219499_at | 4.291814815 | 0.867588968 |
| 225884_s_at | 8.046555556 | 0.690815145 | 222895_s_at | 9.286111111 | 2.657360436 |
| 235453_at | 2.771703704 | 0.499015555 | 202746_at | 9.989222222 | 1.384098355 |
| 210983_s_at | 5.356074074 | 0.830564263 | 1566970_at | 3.093407407 | 1.212494417 |
| 222962_s_at | 5.373814815 | 1.14924052 | 213564_x_at | 12.88977778 | 0.653111095 |
| 208296_x_at | 8.387 | 1.646044046 | 207912_s_at | 3.473148148 | 2.246697657 |
| 215591_at | 3.484296296 | 0.916086862 | 215670_s_at | 2.722074074 | 0.570447997 |
| 209140_x_at | 11.60314815 | 1.621305972 | 1563420_at | 2.360962963 | 0.261750245 |
| 201081_s_at | 3.924740741 | 0.51258137 | 238959_at | 3.313481481 | 0.504845016 |
| 219681_s_at | 9.266518519 | 1.129900655 | 226498_at | 5.325814815 | 1.070877822 |
| 210972_x_at | 7.295407407 | 2.54768316 | 205426_s_at | 4.347814815 | 0.85408246 |
| 224153_s_at | 6.481814815 | 0.739652103 | 201030_x_at | 12.75214815 | 0.73563864 |
| 211911_x_at | 10.68392593 | 1.692384047 | 213295_at | 9.315777778 | 0.994027756 |
| 231827_at | 6.686185185 | 0.967367245 | 239834_at | 5.532407407 | 1.012674648 |
| 211991_s_at | 10.17951852 | 1.908868621 | 210325_at | 5.896407407 | 2.83225006 |
| 224588_at | 8.316703704 | 4.210382303 | 226504_at | 4.700777778 | 1.158195823 |
| 230968_at | 6.290518519 | 1.345499689 | 235688_s_at | 6.055777778 | 1.52548969 |
| 1559224_at | 3.077259259 | 0.917557652 | 232617_at | 10.36885185 | 1.611772906 |
| 210260_s_at | 7.369296296 | 1.777256914 | 39582_at | 8.727962963 | 0.994076513 |
| 223699_at | 3.626074074 | 2.333047329 | | | |
| 205677_s_at | 6.679185185 | 1.021482258 | | | |
| 1557759_at | 3.073518519 | 0.261932518 | | | |
| 235242_at | 8.720703704 | 1.137808684 | | | |
| 229046_s_at | 2.742814815 | 0.703204316 | | | |
| 227316_at | 8.357666667 | 0.738688963 | | | |
| 213975_s_at | 12.12581481 | 1.52359446 | | | |
| 210915_x_at | 6.363592593 | 3.050893365 | | | |
| 1555487_a_at | 3.920407407 | 1.090175401 | | | |
| 208268_at | 6.520962963 | 2.003670353 | | | |
| 1561042_at | 3.179518519 | 1.267582872 | | | |
| 227135_at | 7.392111111 | 1.210907363 | | | |
| 222802_at | 3.25937037 | 1.306707788 | | | |
| 232053_x_at | 2.782148148 | 0.848160575 | | | |
| 223168_at | 7.757444444 | 1.16521323 | | | |
| 206978_at | 6.884222222 | 2.341644513 | | | |
| 217501_at | 9.457777778 | 0.545973255 | | | |
| 212776_s_at | 3.821074074 | 1.104182396 | | | |
| 211756_at | 2.676962963 | 0.731265893 | | | |
| 235239_at | 3.072407407 | 0.633552697 | | | |
| 203868_s_at | 10.83881481 | 1.776208733 | | | |
| 222943_at | 3.357 | 1.884710445 | | | |
| 211742_s_at | 5.958185185 | 2.003553862 | | | |
| 204806_x_at | 9.718259259 | 1.447679485 | | | |

TABLE 4
Pearson analysis

| ProbeSetId | Description |
|---|---|
| 243010_at | musashi homolog 2 (Drosophila) |
| 224153_s_at | |
| 218363_at | exonuclease 3′-5′ domain-like 2 |
| 231229_at | histone linker H1 domain, spermatid-specific 1 |
| 209774_x_at | chemokine (C—X—C motif) ligand 2 |
| 228786_at | |
| 209606_at | pleckstrin homology, Sec7 and coiled-coil |
| | domains, |
| 236681_at | homeobox D13 |
| 224908_s_at | tubulin tyrosine ligase |
| 205204_at | neuromedin B |
| 217995_at | sulfide quinone reductase-like (yeast) |
| 235391_at | family with sequence similarity 92, member A1 |
| 204078_at | |
| 210538_s_at | baculoviral IAP repeat-containing 3 |
| 205890_s_at | ubiquitin D |
| 200905_x_at | major histocompatibility complex, class I, E |
| 212654_at | tropomyosin 2 (beta) |
| 206035_at | v-rel reticuloendotheliosis viral oncogene |
| | homolog |
| 225502_at | dedicator of cytokinesis 8 |
| 201010_s_at | thioredoxin interacting protein |
| 208478_s_at | BCL2-associated X protein |
| 212349_at | protein O-fucosyltransferase 1 |
| 228812_at | |
| 225853_at | glucosamine-phosphate N-acetyltransferase 1 |
| 211700_s_at | trophinin |
| 205696_s_at | GDNF family receptor alpha 1 |
| 235421_at | mitogen-activated protein kinase kinase kinase 8 |
| 237439_at | ubiquitin specific peptidase 43 |
| 223819_x_at | COMM domain containing 5 |
| 229598_at | COBL-like 1 |
| 1563497_at | ubiquitin specific peptidase 25 |
| 217885_at | importin 9 |
| 236627_at | |
| 232019_at | zinc finger protein 694 |
| 204897_at | prostaglandin E receptor 4 (subtype EP4) |
| 232311_at | beta-2-microglobulin |
| 212877_at | kinesin 2 |
| 204233_s_at | choline kinase alpha |
| 227035_x_at | |
| 244393_x_at | aldo-keto reductase family 1, member C-like 2 |
| 209849_s_at | RAD51 homolog C (S. cerevisiae) |
| 205273_s_at | pitrilysin metallopeptidase 1 |
| 200904_at | major histocompatibility complex, class I, E |
| 226043_at | G-protein signalling modulator 1 (AGS3-like, C. el |
| 1556049_at | reticulon 4 |
| 225882_at | solute carrier family 35, member B4 |
| 225432_s_at | CSRP2 binding protein |
| 214901_at | zinc finger protein 8 (clone HF.18) |
| 1555852_at | transporter 1, ATP-binding cassette, sub-family B |
| 225736_at | F-box protein 22 |
| 208296_x_at | tumour necrosis factor, alpha-induced protein 8 |
| 211603_s_at | ets variant gene 4 (E1A enhancer binding protein, |
| 209894_at | leptin receptor |
| 204806_x_at | major histocompatibility complex, class I, F |
| 239486_at | |
| 216438_s_at | thymosin, beta 4, X-linked |
| 235473_at | mediator of RNA polymerase II transcription, subun |
| 1561042_at | integrin, beta 1 (fibronectin receptor, beta polyp |
| 217456_x_at | major histocompatibility complex, class I, E |
| 204116_at | interleukin 2 receptor, gamma (severe combined imm |
| 218807_at | vav 3 oncogene |
| 212671_s_at | major histocompatibility complex, class II, DQ alp |
| 241671_x_at | |
| 202546_at | vesicle-associated membrane protein 8 (endobrevin) |
| 91684_g_at | exosome component 4 |
| 205419_at | Epstein-Barr virus induced gene 2 (lymphocyte-spec |
| 238925_at | syntrophin, beta 2 (dystrophin-associated protein |
| 228604_at | family with sequence similarity 76, member A |
| 241670_x_at | |
| 207651_at | G protein-coupled receptor 171 |
| 223593_at | aminoadipate aminotransferase |
| 205027_s_at | mitogen-activated protein kinase kinase kinase 8 |
| 201009_s_at | thioredoxin interacting protein |
| 202828_s_at | matrix metallopeptidase 14 (membrane-inserted) |
| 228376_at | glycoprotein, alpha-galactosyltransferase 1 |
| 216920_s_at | |
| 221265_s_at | chromosome 15 open reading frame 44 |
| 221875_x_at | major histocompatibility complex, class I, F |
| 243502_at | gap junction protein, alpha 7, 45 kDa (connexin 45) |
| 210260_s_at | tumour necrosis factor, alpha-induced protein 8 |
| 202544_at | glia maturation factor, beta |
| 223646_s_at | chromosome Y open reading frame 15B |
| 200615_s_at | adaptor-related protein complex 2, beta 1 subunit |
| 235688_s_at | TNF receptor-associated factor 4 |
| 209567_at | RRS1 ribosome biogenesis regulator homolog (S. cer |
| 213008_at | KIAA1794 |
| 211529_x_at | HLA-G histocompatibility antigen, class I, G |
| 204410_at | eukaryotic translation initiation factor 1A, Y-lin |
| 223746_at | serine/threonine kinase 4 |
| 226778_at | chromosome 8 open reading frame 42 |
| 223945_x_at | |
| 223168_at | ras homolog gene family, member U |
| 209140_x_at | major histocompatibility complex, class I, B |
| 204266_s_at | choline kinase alpha |
| 211595_s_at | mitochondrial ribosomal protein S11 |
| 208799_at | proteasome (prosome, macropain) subunit, beta |
| | type |
| 213539_at | CD3d antigen, delta polypeptide (TiT3 complex) |
| 236203_at | major histocompatibility complex, class II, DQ |
| | alp |
| 208885_at | lymphocyte cytosolic protein 1 (L-plastin) |
| 207397_s_at | homeobox D13 |

TABLE 5
Multivariate Baldi gene list

| ProbeSet Id | Description |
|---|---|
| 1555852_at | transporter 1, ATP-binding cassette, sub-family B |
| 1559584_a_at | mitogen-activated protein kinase 12 |
| 200905_x_at | ceruloplasmin (ferroxidase) |
| 202688_at | Pvt1 oncogene homolog, MYC activator (mouse) |
| 204233_s_at | zinc finger CCCH-type containing 12D |
| 205419_at | chromosome 16 open reading frame 54 |
| 206082_at | integrin, beta 1 (fibronectin receptor, beta polyp |
| 207175_at | Janus kinase 2 (a protein tyrosine kinase) |
| 209774_x_at | ubiquitin specific peptidase 25 |
| 210915_x_at | major histocompatibility complex, class I, E |
| 212587_s_at | major histocompatibility complex, class I, E |
| 212813_at | thioredoxin interacting protein |
| 215313_x_at | thioredoxin interacting protein |
| 218805_at | beta-2-microglobulin |
| 223593_at | vesicle-associated membrane protein 8 (endobrevin) |
| 226218_at | tumour necrosis factor (ligand) superfamily, member |
| 227550_at | chemokine (C-X-C motif) ligand 9 |
| 228812_at | interferon regulatory factor 8 |
| 232311_at | |
| 235688_s_at | interleukin 2 receptor, gamma (severe combined imm |
| 241671_x_at | choline kinase alpha |
| 1556340_at | CD52 antigen (CAMPATH-1 antigen) |
| 1561042_at | collagen, type IX, alpha 3 |
| 201009_s_at | colony stimulating factor 2 receptor, beta, low-af |
| 203915_at | neuromedin B |
| 204661_at | Epstein-Barr virus induced gene 2 (lymphocyte-spec |
| 205668_at | lymphocyte antigen 75 |
| 206134_at | GDNF family receptor alpha 1 |
| 207651_at | CD8 antigen, alpha polypeptide (p32) |
| 209795_at | ubiquitin D |
| 210972_x_at | HLA complex P5 |
| 212588_at | ADAM-like, decysin 1 |
| 213193_x_at | galactosidase, beta 1-like |
| 216438_s_at | protein tyrosine phosphatase type IVA, member 3 |
| 218807_at | granzyme K (granzyme 3, tryptase II) |
| 224896_s_at | adiponectin, C1Q and collagen domain containing |
| 226219_at | G protein-coupled receptor 171 |
| 228017_s_at | tumour necrosis factor, alpha-induced protein 8 |
| 229152_at | pleckstrin homology, Sec7 and coiled-coil domains, |
| 232843_s_at | alcohol dehydrogenase IB (class I), beta polypepti |
| 236203_at | chemokine (C-X-C motif) ligand 2 |
| 243010_at | CD69 antigen (p60, early T-cell activation antigen |
| 1558034_s_at | leptin receptor |
| 1562031_at | tumour necrosis factor, alpha-induced protein 8 |
| 201010_s_at | baculoviral IAP repeat-containing 3 |
| 204057_at | T cell receptor beta variable 19 |
| 204724_s_at | T cell receptor delta variable 2 |
| 205696_s_at | ets variant gene 4 (E1A enhancer binding protein, |
| 206540_at | trophinin |
| 208296_x_at | T cell receptor beta variable 3-1 |
| 209894_at | protein tyrosine phosphatase, receptor type, C |
| 211603_s_at | protein tyrosine phosphatase, receptor type, C |
| 212592_at | immunoglobulin J polypeptide, linker protein for i |
| 213539_at | tropomyosin 2 (beta) |
| 216920_s_at | major histocompatibility complex, class II, DQ alp |
| 222838_at | junctional adhesion molecule 3 |
| 224908_s_at | T cell receptor beta constant 1 |
| 227265_at | CD3d antigen, delta polypeptide (TiT3 complex) |
| 228071_at | ADAM metallopeptidase domain 12 (meltrin alpha) |
| 229598_at | killer cell lectin-like receptor subfamily B, memb |
| 235276_at | major histocompatibility complex, class I, A |
| 236627_at | thymosin, beta 4, X-linked |
| 244061_at | |
| 1558290_a_at | complement component 3 |
| 1563497_at | |
| 201891_s_at | GTPase, IMAP family member 5 |
| 204078_at | vav 3 oncogene |
| 205159_at | SLAM family member 7 |
| 205758_at | glucosidase, beta, acid 3 (cytosolic) |
| 206574_s_at | polymerase (RNA) I polypeptide B, 128 kDa |
| 209606_at | aminoadipate aminotransferase |
| 210260_s_at | tubulin tyrosine ligase |
| 211700_s_at | tubulin tyrosine ligase |
| 212654_at | dedicator of cytokinesis 8 |
| 213790_at | solute carrier family 35, member B4 |
| 217767_at | interleukin 7 receptor |
| 222943_at | Rho GTPase activating protein 30 |
| 225502_at | fibrinogen-like 2 |
| 227346_at | zinc finger protein, subfamily 1A, 1 (Ikaros) |
| 228094_at | heparan sulfate (glucosamine) 3-O-sulfotransferase |
| 231229_at | |
| 235391_at | chromosome 20 open reading frame 58 |
| 236681_at | GTPase, IMAP family member 7 |
| 244393_x_at | adhesion molecule, interacts with CXADR antigen 1 |
| 1559263_s_at | |
| 200904_at | |
| 202546_at | chromosome 4 open reading frame 7 |
| 204116_at | COBL-like 1 |
| 205204_at | histone linker H1 domain, spermatid-specific 1 |
| 205890_s_at | |
| 206666_at | beta-2-microglobulin |
| 209613_s_at | dedicator of cytokinesis 8 |
| 210538_s_at | epithelial stromal interaction 1 (breast) |
| 211796_s_at | family with sequence similarity 92, member A1 |
| 212671_s_at | mitogen-activated protein kinase kinase kinase 8 |
| 214470_at | TNF receptor-associated factor 4 |
| 218499_at | major histocompatibility complex, class II, DQ alp |
| 223403_s_at | |
| 225882_at | homeobox D13 |
| 227361_at | |
| 228786_at | |
| 231929_at | musashi homolog 2 (Drosophila) |
| 235421_at | Rho GTPase activating protein 15 |
| 238834_at | aldo-keto reductase family 1, member C-like2 |
| 34210_at | CD52 antigen (CAMPATH-1 antigen) |

TABLE 6
SPCA

| ProbeSet Id | Description |
|---|---|
| 1552584_at | interleukin 12 receptor, beta 1 |
| 1558972_s_at | chromosome 6 open reading frame 190 |
| 1559584_a_at | chromosome 16 open reading frame 54 |
| 1563473_at | protein phosphatase 1, regulatory (inhibitor) subu |
| 203915_at | chemokine (C-X-C motif) ligand 9 |
| 204116_at | interleukin 2 receptor, gamma (severe combined imm |
| 204533_at | chemokine (C-X-C motif) ligand 10 |
| 205242_at | chemokine (C-X-C motif) ligand 13 (B-cell chemoatt |
| 205488_at | granzyme A (granzyme 1, cytotoxic T-lymphocyte-ass |
| 205758_at | CD8 antigen, alpha polypeptide (p32) |
| 205890_s_at | ubiquitin D |
| 206134_at | ADAM-like, decysin 1 |
| 206666_at | granzyme K (granzyme 3, tryptase II) |
| 207651_at | G protein-coupled receptor 171 |
| 209606_at | pleckstrin homology, Sec7 and coiled-coil domains, |
| 209795_at | CD69 antigen (p60, early T-cell activation antigen |
| 210915_x_at | T cell receptor beta variable 19 |
| 210972_x_at | T cell receptor delta variable 2 |
| 211796_s_at | T cell receptor beta variable 3-1 |
| 212592_at | immunoglobulin J polypeptide, linker protein for i |
| 212999_x_at | major histocompatibility complex, class II, DQ bet |
| 213193_x_at | T cell receptor beta constant 1 |
| 213539_at | CD3d antigen, delta polypeptide (TiT3 complex) |
| 214617_at | perforin 1 (pore forming protein) |
| 216920_s_at | |
| 217147_s_at | T cell receptor associated transmembrane adaptor 1 |
| 222838_at | SLAM family member 7 |
| 222895_s_at | B-cell CLL/lymphoma 11B (zinc finger protein) |
| 226218_at | interleukin 7 receptor |
| 227346_at | zinc finger protein, subfamily 1A, 1 (Ikaros) |
| 228362_s_at | |
| 229152_at | chromosome 4 open reading frame 7 |
| 231577_s_at | guanylate binding protein 1, interferon-inducible, |
| 236280_at | |

TABLE 7

| | Value in | | Value in | | Value |
|---|---|---|---|---|---|
| ProbesetId | metagene | ProbesetId | metagene | ProbesetId | in metagene |
| 243010_at | 4.1981 | 244393_x_at | 4.1734 | 243502_at | 2.9835 |
| 224153_s_at | 6.11975 | 209849_s_at | 10.46455 | 210260_s_at | 8.21145 |
| 218363_at | 7.46445 | 205273_s_at | 7.95315 | 202544_at | 11.7167 |
| 231229_at | 5.0826 | 200904_at | 12.81605 | 223646_s_at | 2.75825 |
| 209774_x_at | 7.7947 | 226043_at | 3.91835 | 200615_s_at | 5.74175 |
| 228786_at | 3.95185 | 1556049_at | 7.62715 | 235688_s_at | 5.4642 |
| 209606_at | 7.1185 | 225882_at | 5.8835 | 209567_at | 7.6344 |
| 236681_at | 5.9989 | 225432_s_at | 7.25855 | 213008_at | 7.3653 |
| 224908_s_at | 9.6059 | 214901_at | 3.58985 | 211529_x_at | 8.97615 |
| 205204_at | 4.00725 | 1555852_at | 7.71885 | 204410_at | 2.7465 |
| 217995_at | 9.48115 | 225736_at | 6.634 | 223746_at | 8.9655 |
| 235391_at | 7.133 | 208296_x_at | 9.27055 | 226778_at | 3.78595 |
| 204078_at | 5.4736 | 211603_s_at | 4.00935 | 223945_x_at | 6.9724 |
| 210538_s_at | 8.39055 | 209894_at | 7.57655 | 223168_at | 8.32455 |
| 205890_s_at | 10.7217 | 204806_x_at | 10.7746 | 209140_x_at | 12.58685 |
| 200905_x_at | 11.51055 | 239486_at | 7.65935 | 204266_s_at | 4.98385 |
| 212654_at | 4.04655 | 216438_s_at | 13.89245 | 211595_s_at | 5.8073 |
| 206035_at | 6.382 | 235473_at | 7.0424 | 208799_at | 10.0343 |
| 225502_at | 7.805 | 1561042_at | 2.54365 | 213539_at | 8.76235 |
| 201010_s_at | 12.7636 | 217456_x_at | 10.31775 | 236203_at | 6.30605 |
| 208478_s_at | 3.6941 | 204116_at | 7.8395 | 208885_at | 11.59615 |
| 212349_at | 7.0306 | 218807_at | 8.1423 | 207397_s_at | 2.3551 |
| 228812_at | 7.94125 | 212671_s_at | 10.0539 | | |
| 225853_at | 9.85825 | 241671_x_at | 4.81515 | | |
| 211700_s_at | 4.07 | 202546_at | 10.8191 | | |
| 205696_s_at | 4.4725 | 91684_g_at | 4.68685 | | |
| 235421_at | 6.0634 | 205419_at | 7.50055 | | |
| 237439_at | 5.3148 | 238925_at | 5.6971 | | |
| 223819_x_at | 5.4128 | 228604_at | 6.10995 | | |
| 229598_at | 7.14425 | 241670_x_at | 4.296 | | |
| 1563497_at | 7.0658 | 207651_at | 8.8235 | | |
| 217885_at | 7.606 | 223593_at | 3.5633 | | |
| 236627_at | 2.62845 | 205027_s_at | 4.81415 | | |
| 232019_at | 5.94545 | 201009_s_at | 12.1921 | | |
| 204897_at | 10.1441 | 202828_s_at | 3.60535 | | |
| 232311_at | 7.71315 | 228376_at | 8.1722 | | |
| 212877_at | 4.82955 | 216920_s_at | 6.8166 | | |
| 204233_s_at | 4.32305 | 221265_s_at | 6.9674 | | |
| 227035_x_at | 7.60835 | 221875_x_at | 11.55795 | | |

TABLE 8

| ProbeSetId | Value in Metagene |
|---|---|
| 1555852_at | 7.71885 |
| 1556340_at | 2.3965 |
| 1558034_s_at | 8.0664 |
| 1558290_a_at | 9.06445 |
| 1559263_s_at | 9.4082 |
| 1559584_a_at | 10.0188 |
| 1561042_at | 2.54365 |
| 1562031_at | 7.295 |
| 1563497_at | 7.0658 |
| 200904_at | 12.81605 |
| 200905_x_at | 11.51055 |
| 201009_s_at | 12.1921 |
| 201010_s_at | 12.7636 |
| 201891_s_at | 13.8519 |
| 202546_at | 10.8191 |
| 202688_at | 9.50265 |
| 203915_at | 11.036 |
| 204057_at | 10.9814 |
| 204078_at | 5.4736 |
| 204116_at | 7.8395 |
| 204233_s_at | 4.32305 |
| 204661_at | 8.99365 |
| 204724_s_at | 3.4864 |
| 205159_at | 9.46255 |
| 205204_at | 4.00725 |
| 205419_at | 7.50055 |
| 205668_at | 9.17675 |
| 205696_s_at | 4.4725 |
| 205758_at | 9.1644 |
| 205890_s_at | 10.7217 |
| 206082_at | 7.52315 |
| 206134_at | 9.277 |
| 206540_at | 5.5016 |
| 206574_s_at | 4.20995 |
| 206666_at | 8.16675 |
| 207175_at | 7.7285 |
| 207651_at | 8.8235 |
| 208296_x_at | 9.27055 |
| 209606_at | 7.1185 |
| 209613_s_at | 8.7372 |
| 209774_x_at | 7.7947 |
| 209795_at | 8.7526 |
| 209894_at | 7.57655 |
| 210260_s_at | 8.21145 |
| 210538_s_at | 8.39055 |
| 210915_x_at | 7.83255 |
| 210972_x_at | 8.64305 |
| 211603_s_at | 4.00935 |
| 211700_s_at | 4.07 |
| 211796_s_at | 8.42035 |
| 212587_s_at | 11.9281 |
| 212588_at | 9.4714 |
| 212592_at | 10.15105 |
| 212654_at | 4.04655 |
| 212671_s_at | 10.0539 |
| 212813_at | 11.41345 |
| 213193_x_at | 8.2094 |
| 213539_at | 8.76235 |
| 213790_at | 8.2969 |
| 214470_at | 7.66275 |
| 215313_x_at | 12.5728 |
| 216438_s_at | 13.89245 |
| 216920_s_at | 6.8166 |
| 217767_at | 10.25435 |
| 218499_at | 8.77755 |
| 218805_at | 11.4793 |
| 218807_at | 8.1423 |
| 222838_at | 8.6475 |
| 222943_at | 2.34225 |
| 223403_s_at | 9.84385 |
| 223593_at | 3.5633 |
| 224896_s_at | 10.35155 |
| 224908_s_at | 9.6059 |
| 225502_at | 7.805 |
| 225882_at | 5.8835 |
| 226218_at | 10.44915 |
| 226219_at | 7.24985 |
| 227265_at | 10.99465 |
| 227346_at | 9.9391 |
| 227361_at | 6.83655 |
| 227550_at | 8.60875 |
| 228017_s_at | 2.86415 |
| 228071_at | 8.78515 |
| 228094_at | 7.5848 |
| 228786_at | 3.95185 |
| 228812_at | 7.94125 |
| 229152_at | 8.7441 |
| 229598_at | 7.14425 |
| 231229_at | 5.0826 |
| 231929_at | 9.37265 |
| 232311_at | 7.71315 |
| 232843_s_at | 9.8371 |
| 235276_at | 8.5938 |
| 235391_at | 7.133 |
| 235421_at | 6.0634 |
| 235688_s_at | 5.4642 |
| 236203_at | 6.30605 |
| 236627_at | 2.62845 |
| 236681_at | 5.9989 |
| 238834_at | 2.2871 |
| 241671_x_at | 4.81515 |
| 243010_at | 4.1981 |
| 244061_at | 7.1302 |
| 244393_x_at | 4.1734 |
| 34210_at | 9.9364 |

TABLE 9
18 PS gene list, target sequences

| | |
|---|---|
| Probe Set ID | Target sequence |

| | |
|---|---|
| 1562031_at | atgttcactgtatgtgccaagcctaatatgagagctatgtattatagagtttatgctacagccctaccttcaggaaacttat |
| SEQ ID | ctactggacaaacaaaaattttcaaatatacaaaaaattctaaatcgaacattgtaattatctagcataggcaaatatagac |
| NO: 1 | agtaacagacaggtttacaattattaagaaagggcagccagg |
| 1563497_at | taccatagcaagttcttatcctttgttaatatattataaatacgacttttttcctttacctgtcacagtaatatgccaattt |
| SEQ ID | ttggtgtcgctttagtaatttttttgggggggactgtcaaatttcagtaaactttataggctatagtttacaatttaaggtc |
| NO: 2 | tttcctcagttgtctcctaaaaaattttgtggaaatcgaaatttcttttcaattgatcgtcttgtgcagccctttaaaaaat |
| | gcgttttgcattcatgtctttgcagtacaaatctgtatctttcattttgggagtgactgtcatagatgggtaatataaagtg |
| | acaacacacacaccagggccagattgcgtgggttctagctagggacctagagctggttacttgatttctctctgttccattt |
| | actatctg |
| 202290_at | agggctcatcgacatcgagaaccccaaccgggtggcacagacaaccaaaaaggtcacacaactggatctggacgggccaaag |
| SEQ ID | gagctttcgaggagagaacgagaagagattgagaagcagaaggcaaaagagcgttacatgaaaatgcacttggccgggaaga |
| NO: 3 | cagagcaagccaaggctgacctggcccggctggccatcatccggaaacagcgggaggaggctgcccggaagaaggaagagga |
| | aaggaaagcaaaagacgatgccacattgtcaggaaaacgaatgcagtcactctccctgaataagtaactgcgacccgtggga |
| | ggagatgccggggacctgggccgcgctgccaggacctctgctgtgtctcgcccaccctgtgccctggcgccgctgcaacagc |
| | ccctcatggccaggagccccccatgcctgggcctcctcttcatcttggcacagaaattgtttgggggatgggggggggactg |
| | ggggaggggtagctgctatctttgagacag |
| 203992_s_at | aggaaataacccagttctgcaccactggtttttgtagctatctcgtaaggctgctggctgaaaactgtgtctatgcaacctt |
| SEQ ID | ccaagtgcggagtgtcaaccaactggacgggagagagtactgctcctactccaggactctcacaaagctgatgagctgtact |
| NO: 4 | tcagaaaaaaataataatttccatgttttgtatatatctgacaaaactggcaacatcttacagactactgacttgaagacaa |
| | cctcttttatatttctctatttctgggctgatgaatttgttttcatctgtcttttcccccttcagaattttccttggaaaaa |
| | aaatactagcctagctggtcatttctttgtaaggtagttagcaattttaagtctttctttggtcaacttttttttaatgtga |
| | aaagttaggtaagacacttttttactgcttttatgtttttctgtcttgttttgagaccatgatggttacacttttggttcct |
| | a |
| 205518_s_at | tcaagaattcactgatctgatgcaaaataaaaatttatcattacatcttgaacccaggaagcttacagcaaagagactatgc |
| SEQ ID | tttatgacgtcagcaatagataattccacgttgcctttgtgatttgtatatatagcttacatttgtggaccactacatagcc |
| NO: 5 | agattcaaaaatattttacttgttccatccacagttctctacagaaagaaccaatgaacccaataggaacaaattctctgtg |
| | gaaaacaaagcatagctgtagtagatacgaatccaatcacagaggaaacaggaagagaaaaacatccaagactacagtgaaa |
| | actggaaatggtctgttttcgtgatattcgtatgattaagatgcaaattttttcttaggaaaatgtgattgttaactagcat |
| | tctgttttacatgttgacatttctaacacacacaccactgatttgaacttcaaaatttattttctgattatatatgctaggt |
| | ctgattctgaag |
| 206618_at | gaagcatatccagagggcgaaagatatctctccattgtgcatctgcctcttttgacgttggaagacacatgtcttactcccc |
| SEQ ID | aaagggagcccagcactgggagccttcttgatgatctcaaaaataatagctattcaagaaaatcaccaagtgactgtgaaac |
| NO: 6 | cgtcagttcggaaggctggttagaacatgtgggagcaacatgaatgttctacaaaagtttaaagcagagattgtttcaaatg |
| | ggtgtagtagatattactgaaaaccaaaaaagagtgagattgtcagtgtaagaatgtgatttaatgtttgtagtgcttacaa |
| | ttttgtgtaccaactggatgactaaaaagagtaaaataacttaattaatagctcatattttatgtgtgaaaacatgttagtg |
| | aacatatataatcaaaatagatttcattgctattgcatagtctc |
| 212684_at | gactatttcaaagctactgttcctagtccagctttaagtttcggtaagaaacatgctgttttgtttcatgatttcgttaatt |
| SEQ ID | atggaaatttggcattgagggattattttattgagggtagaagagattccagaatcatcatctgtgatgatggtgtccttta |
| NO: 7 | gggctcttggagcagccagaccatgtttccaagagaaacctggtgatattgccagcagaccccctgccatccccccnagttg |
| | tcctggggctgaatgggcaaatctgtccaaacagctagtaaccggctgtgagggagagggtcagaagcacttagcgttggcc |
| | tctgattgctgtcctctcttgtcctcttcccactccaatgatgaaaatgattttctctaaatgcctgggtaaggatgctttc |
| | aaggagctcacttggcctgctt |
| 212776_s_at | tctcctcgtggatcgtgtatcccagcggcaaggtgtatgtggcagcngtgcgcctggagcgtgtggtgctgacctgtgagct |
| SEQ ID | atgccggccctgggcagaggtgcgctggaccaaggatggagaggaggtggtggagagccccgcgctgctcctgcagaaggaa |
| NO: 8 | gacactgtccgccgcctggtgctgcccgctgtccagctcgaggactccggcgagtacttgtgtgaaattgacgatgagtcgg |
| | cctcctt |
| 215666_at | gaaaaatccgtggaactgaagctgagatctttagtacgtggagtcaccttacagatacagagcatttatgcggtattcattg |
| SEQ ID | gtgcctaaagaactttaggcatcctctggaaaaccggcc |
| NO: 9 | |
| 219551_at | gaacaggtgaccataactctgccaaatatagaaagttgaaggaagtagtaaaattcagtatcgtaaagaacaacagcaacaa |
| SEQ ID | caaatgtggaattcagccaggactcccaatcttgtaaaacattctccatctgaagataagatgtccccagcatctccaatag |
| NO: 10 | atgatatcgaaagagaactgaaggcagaagctagtctaatggaccagatgagtagttgtgatagttcatcagattccaaaag |
| | ttcatcatcttcaagtagtgaggatagttctagtgactcagaagatgaagattgcaaatcctctacttctgatacagggaat |
| | tgtgtctcaggacatcctaccatgacacagtacaggattcctgatatagatgccagtcataatagatttcgagacaacagtg |
| | gccttctgatgaatacttt |
| 220301_at | ataagtcatttctaatctttgtataaaacagaagtgagcaagatgaatcagaaaaaaggtgttttgtattttaaaggtaaca |
| SEQ ID | gataaccaggtgattgaatctaagacaggctgtaagcatcgctgagaaactaaaaggacttttgacttttatctggatagac |
| NO: 11 | atttctacagtaaaatcatggaaaggcatcagcattgcaaagtagcatctaggtagaaatcaggccaaaattaagctgtggt |
| | ttccctctgagtagtgggaatagagaaaattaggaaattgtggttatgtgaatatttctttaaaacttttatgtacattata |
| | gtttattgcttcatatttaagtttagtttttaaggtaaaatgttattttgaacaaaaagacacttataattttccataccta |
| | ttttcaactgaaggcaacttgtaagatttaactcagtcaataacatactggttttactcatc |
| 223095_at | tgcctgtagcattccagagctcactgcccttctagatgtgccttcccgcttggcttccagcggcttgtgctcactctgtctg |
| SEQ ID | ccaggtatgagaagaacacgtaagaccgccaccacactcaccctccctcaaggccctgtgccataggggtggccacccgacc |
| NO: 12 | tgcccccagaacttttggatactggaggcagttgcataggtctccctctctgggcaccaggactcagtccagcccaagacta |
| | ctctgggcagctcccatcccagtctggggccatttgcagactcaggaaaggatttctacagtgttctataaaagccaaaaga |
| | gagagtgggtttgggaagagtgagggtggttggggagaggggaccgatgtgcctcattgtttagtggtgattacaaatatgc |
| | ttttctggataaagtttggttgtttgc |
| 227927_at | tacctctgtcgacctgtgaaactgaacaaacagcttatctgcccctaatgtgaaatgatgggacagacatagaataacnact |
| SEQ ID | acagtgattctagttcaaaatgagggaacatggaggggataaagaagtcantaacccaaaatagtttggaaatggagctggg |
| NO: 13 | caaaatccagcaggagtttcttagttaggatccacagcctgggactgaccctctgtcctgtgggtctttgcctctgggctct |
| | ctgctctgcatttcttgaaaccattatta |
| 228128_x_at | gatgttgaaacctgtttggcaccttctggaagctaccaaaaaaatgacactccattgaagtgcttaaaagctgttctcataa |
| SEQ ID | gaattctactggcctattgtaaaaaagaaaaaaaaaangaaaaagaagaaagacacaaagaaaataatctaaacaccaaaaa |
| NO: 14 | ctaaacacaattccaatcctttttctgtacctcacgcgcataaattngctgctcctattttttttnctgttnatgtgttttn |
| | atggatctaagttaaatcttttggcaatatataaaaatgtaaatagtaaactttatttattaagaatgtcatcttttttaat |
| | ttatatttacacaattgttcatctaatttattttttctatacagttttaaatactcagacatattttgctgttcatgatatt |
| | tttatcctgttctcatggatttgttttcccatactgttttctctgatctcaattacaggttggatctcacaaataataatgt |
| | cagagacagaaatattttgccactgttgattactatac |
| 228812_at | gagacttcattggtgatacactcaatttttactgggtaattagctaataatgttggtcactgtctcacagttcaagtagctt |
| SEQ ID | taagatgatgtggcaaggaaaacacaaagcttttgggtaaccagcgttcttaaatgtatggtttttgaccaggtgaaccctt |
| NO: 15 | tagaagtgatttctgttttaaaagtatgtacttaaaatacctttggctgtgatgaatgtagatcccagcagaataccaaaat |
| | cctattttttttgactgagtatttgtagatgcttaatgactgaaatgaatttggaggcactgatgaaagtgatttttttnaa |
| | gttctcaggtactgttcaattat |
| 229022_at | gaaacacttttagagcacttgaggaacctaaaagatgactggttcagcattttgtgtggtagataagaaagaaattatcaca |
| SEQ ID | aaaaatcagaaatgaacagtgagagaaaaataggaccccagacagtttataccttccatttgctgttttaaaagtgtgagcc |
| NO: 16 | tgccaagtcaacaagtatgcctttagcgcacatgtaaatagcctgcacttcctaaatctcgtgtggcctcccatggttacat |
| | tcttcaaaggtnaactgagttgagaggaagattcagcatttaaaagagaagggttgaaaaagatnntgtgtgtgtgtgtgtg |
| | tgtgtttaattggcccagggttacttaaataaatcataaccattttgccacattctgtaactgtttagctaaggtcaaatta |
| | agtttaccctatggattttgtttcatcttttgtttcgtgtatatactgtttgcctttttca |
| 233976_at | tgagtcttttgaacacaggtatgaagtacaaagtgaatctaaaccaatgttacccctaccatacaggtatgttgtgttcaca |
| SEQ ID | aacacacacacacacacacacacaaattaaacatggtggaaatttttgacaaanttaaccacattaaatattctgtccatta |
| NO: 17 | agattccccaaagatggtgganngatngcagngttagtgngagaagatatttgcagcatgcaaggcaagatgacaatgcact |
| | aataacaaaatttcataaaacaatggacttggggcttgggaaaaataattatgacatgctttctgaagtgtccccagatagt |
| | ttcataaaaaatggagcaaataggctaaagaaaaacccatagacatcatgttcaaaagaactgatgacaaggcatgttcata |
| | acccataaaatacaagtggatgcagctaaataactgcaatacttttgaaagagtttggcaggtttcgaagatgtg |
| 244008_at | tacatttcacatttaccagcaagtcagtaaaaaatagtgcttatttacatagtcaatataatttaatgttctaaaaataata |
| SEQ ID | tcttcgatctgcccaatatttaatgtatcatttgagatttttaaaaatgcanccgctccattatgtaaacattaagatatgc |
| NO: 18 | ctatgtttctttaactatacagcctctttacaataaatttcttgatttttgtgcacaggatagtattgcaacctgctattta |
| | gcctttggtgccttagaattattataaatatttaacaatatgtacataatgtaaatactgccaagagatcagtaaggccaaa |
| | tattttctctattcactttttattgctcttgctttctattgctactaaagcctcttttatccagctttgtaatagttccaac |
| | attgtagcgaa |

TABLE 10
250 PS gene list, target sequences

| | |
|---|---|
| Probe Set ID | Target sequence |

| | |
|---|---|
| 1553555_at | gggtctccaggaagatctcccagatgctcctgggtattattctttgctcctgcatctgcactgtcctctgtgtttggtgctt |
| SEQ ID | ttttagcagacctcacttcacagtcacaactgtgctattcatgaataacaatacaaggctcaactggcagaataaagatctc |
| NO: 19 | aatttattttattcctttctcttctgctatctgtggtctgtgcctcctttcctattgtttctggtttcttctgggatgctga |
| | ctgtctccctgggaaggcacatgaggacaatgaaggtctataccagaaactctcgtgaccccagcctggaggcccacattaa |
| | agccctcaagtctcttgtctcctttttctgcttctttgtgatatcatcctgtgttgccttcatctctgtgcccctactgatt |
| | ctgtggcgcgacaaaataggggtgatggtttgtgttgggataatggcagcttgtccctctgggcatgcagccatcctgatct |
| | caggcaatgccaagttgaggagagctgtgatgaccattctgctctgg |
| 1554999_at | gtttacctagtattcatcaccctgcttgttttctctgtgttatgtcttggaaacccttccatgtcaatgtaggtagctgtat |
| SEQ ID | cttctttcagctgctatacagtagtccagtatgtgattgggccatgtttctttttctttttaaaaactattctattggacac |
| NO: 20 | aaacaagttaaatttgtttatactgaaaactcaggcgttttctgagaaagacttagcaaagaagattaagtcccttcttcat |
| | atcaaactctgtttagtaaagtagtaacatatagttgtgggttgctcttgtaaacaggctttactgactgtgaaacttcaac |
| | tttgtaacatgtattttctactctgcacttgatcttagccaaaagggtgagaagcgattctaccctggatagtatatgctgt |
| | aattaagtatctccaagaaggtgtttattagccattctgagta |
| 1556209_at | agattatggcctattgaacaaaatgtgaaactgggagtccacaaagacataaatggaaagatgaagtacctcctgacagagc |
| SEQ ID | aagaatggcactgaacaatgttatatggacaatggctattttattcaagactattacaatagggaaaagagactaggactca |
| NO: 21 | gtctgaactgaaatctgtcaaaacaaaggggggtggggctttaagagtgaaggtgaggaggagatcacacaccgtatgtgtt |
| | tgctaactggctttacccaaaagaaaattaaactttctttgatctgtacaagttgatcaaanaaannnnnnnannnnnnnna |
| | nannnaactaaaacagggtagacaggccaggggaaaaaggcactcagggcacacagcattgcttcaaaatataattctctac |
| | aaacctagttgctaaaactacctgttgtaacct |
| 1556341_s_at | aggctgtgctggacctggcttcatttctgctccaggatggcaggctgtgtcttcttcatggctgcatgaccagcctggggtg |
| SEQ ID | ggtgtgcccggggaaagtaggtgtacactgggagccgtgccatgtttcaggccctgtcctggctctgcctcgggtgcgggac |
| NO: 22 | cttggtcctccacactggctgggtggggctggatgggttgagattcacatgtgctccttacgaccacgggtccctgggtgcc |
| | cgcagcagggcctccacccttcgctgtgtgcatattcgtgtgcttatgagtt |
| 1557458_s_at | accctctccttgagtttctgtgaattaaaatatttgcaaatncanannnnnnnanannnnaaannnnnnnnnnnnnnnnnnn |
| | ncnactgatgctgggagccaaatgctggtgctttgagagtcccggaggccccggggttcccgccccgctggtgtgtatatgt |
| SEQ ID | gtgtctgtgtgagtgtgtgtgtgagtacagatgtgagaaggtggtcacacacagatgggtaagcccactgatctacttgtag |
| NO: 23 | tcactcagtgtaatcattaggctatcttcaaggaatcattgtgcagtcaaaa |
| 1558111_at | aaacagttgatggtgcactcgtagctgcaaaatgaaggacattaaatttggctgtgacattgagtggttacacagaagacct |
| SEQ ID | gcttctacaacatttggaaacattgtgaaacattactattcagttgctatttttccatctcaaaatattnttttttagttgc |
| NO: 24 | tgctcacttatggccacagaacagtccctttttgagtaaagcagcaaggntgcttaatnacatgctatgtggttaacaagat |
| | tcnactgccacntcaaggactttaaataatttcaagattttaaatgtatttatgaaatgaactcattacagcagctaatcaa |
| | tgcaaactttgt |
| 1561226_at | atctccctcagtgcctggcaatgtttgtggttggttgagtgagttaacaggagaccatcttttggccttttttctacctctg |
| SEQ ID | ttttctcttactatacttgcctacatctcatcttct |
| NO: 25 | |
| 1562031_at | atgttcactgtatgtgccaagcctaatatgagagctatgtattatagagtttatgctacagccctaccttcaggaaacttat |
| SEQ ID | ctactggacaaacaaaaattttcaaatatacaaaaaattctaaatcgaacattgtaattatctagcataggcaaatatagac |
| NO: 26 | agtaacagacaggtttacaattattaagaaagggcagccagg |
| 1562249_at | gatgactatctatgaacctaaggaggaaaagttaatttcttttgagggtttgcataatgttttaaaacaaatttatagaaat |
| SEQ ID | atagatgatggaaaaaagcaatagggtaaacctaaattanttaaaaataaataatacatnaggaaataaacagcatagaaaa |
| NO: 27 | gaaacaaaaaagagattgttaccaaaaacnataagtttttttttttttgaaaattctaagaaggtggataaatttctggaat |
| | tttcaatgagcaaaaaagtgaaaagatacaaatgaataaaaaatgaaaaggtagaaattaaaataggtctagtagagattaa |
| | aaataataaaagaatattattaaaaagtattggtggataaaataggcacatatctgaagataaaagccgaattgacccccta |
| | aaaagcagaaaacttacaataacaatcattaatggctttgaatggctatgaaaaaataatatctctgccctcttcactactc |
| | ccatgtaaaataattttacgggtagattttaccaacgtttcaagagagacatgattcccaaatattatgtaa |
| 1563497_at | taccatagcaagttcttatcctttgttaatatattataaatacgacttttttcctttacctgtcacagtaatatgccaattt |
| SEQ ID | ttggtgtcgctttagtaatttttttgggggggactgtcaaatttcagtaaactttataggctatagtttacaatttaaggtc |
| NO: 28 | tttcctcagttgtctcctaaaaaattttgtggaaatcgaaatttcttttcaattgatcgtcttgtgcagccctttaaaaaat |
| | gcgttttgcattcatgtctttgcagtacaaatctgtatctttcattttgggagtgactgtcatagatgggtaatataaagtg |
| | acaacacacacaccagggccagattgcgtgggttctagctagggacctagagctggttacttgatttctctctgttccattt |
| | actatctg |
| 1565544_at | gtagagttctctaaagggtcttgatattagtgggacataattagttctaaactaaatactgctctggtcctgcctaacaaat |
| SEQ ID | cacaaaagcaagatccaaaagaatcaaactgtttccaagtcacttaactatagtccagaaaaaaagctcaagaatattcata |
| NO: 29 | ggaatacaaaaatatccaatacccaaaaaggtaaaattcacaaagtctgccacaaagtctggcatccagttgagaactacca |
| | gacatacaaaaaagcagcagaaaaatataatctataataaagagaaaaatcaatcataaccacccagaactaacacagatat |
| | tagaattagcagataaaatggaaagttattatagctatattctatatagttaaaaaactagaggaaagctcaaagatagtaa |
| | gaagagacatgaaagataataaaaacccaaatcaaacnnnnnnaaatgaaaattatctgaggttaaaaaaatacactggata |
| | gaattaatggcaag |
| 1568752_s_at | agattaacattgacagttcgacaagagagactatcatcaggaacattcaggaacccactgaaacatgttttgaagaagctca |
| SEQ ID | gaaaatagtctatatgcatatggaaagggattcctaccccagatttctaaagtcagaaatgtaccaaaaacttttgaaaact |
| NO: 30 | atgcagtccaacaacagtttctgactacaactcaaaagtttaaatagaaaacagtatattgaaagtggtgggtttgatcttt |
| | ttatttagaaacccacaaaatcagaaacacagtacaaataaaacagaaatcaaactataagttgacttttagttcctaaaaa |
| | gaaacatatttcaaaagcaatggaatctagaattcttataacatgaataacaaaatgtacagcaagcctatgtagttcaat |
| 200924_s_at | acagcctatggaggctccagtcatgctgtgggatgagtccagcttccctgacatcccaggggctgtaagtgccaacatgact |
| SEQ ID | gtgaagggccagagtgaagaccctggctccctcctttccttgttccggcggctgagtgaccagcggagtaaggagcgctccc |
| NO: 31 | tactgcatggggacttccacgcgttctccgctgggcctggactcttctcctatatccgccactgggaccagaatgagcgttt |
| | tctggtagtgcttaactttggggatgtgggcctctcggctggactgcaggcctccgacctgcctgccagcgccagcctgcca |
| | gccaaggctgacctcctgctcagcacccagccaggccgtgaggagggctcccctcctgagctgggacgcctgaaactggagc |
| | ctcacgaagggctgct |
| 201008_s_at | gcaaaggggtttcctcgatttggagctttttttttcttccaccgtcatttctaactcttaaaaccaactcagttccatcatg |
| SEQ ID | gtgatgttcaagaagatcaagtcttttgaggtggtctttaacgaccctgaaaaggtgtacggcagtggcgagaaggtggctg |
| NO: 32 | gccgggtgatagtggaggtgtgtgaagttactcgtgtcaaagccgttaggatcctggcttgcggagtggctaaagtgctttg |
| | gatgcagggatcccagcagtgcaaacagacttcggagtacctgcgctatgaagacacgcttcttctggaagaccagccaaca |
| | ggtgagaatgagatggtgatcatgagacctggaaacaaatatgagtacaagttcggctttgagcttcctcaggggcctctgg |
| | gaacatccttcaaaggaaaatatgggtgtgtagactactgggtgaaggcttttcttgaccgcccgagccagccaactcaaga |
| | gacaaagaaaaactttgaagtagtggatctggtggatgtcaatacccctgattta |
| 201010_s_at | gtgttctcctactgcaaatattttcatatgggaggatggttttctcttcatgtaagtccttggaattgattctaaggtgatg |
| SEQ ID | ttcttagcactttaattcctgtcaaattttttgttctccccttctgccatcttaaatgtaagctgaaactggtctactgtgt |
| NO: 33 | ctctagggttaagccaaaagacaaaaaaaattttactacttttgagattgccccaatgtacagaattatataattctaacgc |
| | ttaaatcatgtgaaagggttgctgctgtcagccttgcccactgtgacttcaaacccaaggaggaactcttgatcaagatgcc |
| | caaccctgtgatcagaacctccaaatactgccatgagaaactagagggcaggtgttcataaaagccctttgaacccccttcc |
| | tgccctgtgttaggagatagggatattggcccctcactgcagctgccagcacttggtcagtcactctcagccatagcacttt |
| | gttcactgtcctgtgtcagagcactgagctccacccttttctgagagttat |
| 201087_at | atcaggactctgacgtctctttggtcttgttgatttacctctgggcatatcccttccccagatctgctcctcccctttcaca |
| SEQ ID | ggtgggatcggcactcagggggtctggaaagaaggtcataagggagcatgataggatttggggcagagggacaggctcctct |
| NO: 34 | ggggaaaccccccagagctctttaccaaggatgaaagaggagccaggccttgggctcctgatgaccagaaaggggccaccgg |
| | ggtctaatggtgacagtccaaaccactccactggcctcctggcagaagccgagtgtgctggggtctccgaagagggtccctc |
| | ctttttgggggaaggtcagcccagcccctccaaaggtctgatgtctccactttcacccgcaggccttaccgctctgtttata |
| | gtgacccaccctagatctt |
| 201115_at | cctaccaggccaccattgatggagtcagatttttggggacatcaggacagaacgtgagtgacattttccgatacagcagcat |
| SEQ ID | ggaggatcacttggagatcctggagtggaccctgcgggtccgtcacatcagccccacagccccggacactctaggttgttac |
| NO: 35 | cccttctacaaaactgacccgttcatcttcccagagtgcccgcatgtctacttttgtggcaacacccccagctttggctcca |
| | aaatcatccgaggtcctgaggaccagacagtgctgttggtgactgtccctgacttcagtgccacgcagaccgcctgccttgt |
| | gaacctgcgcagcctggcctgccagcccatcagcttctcgggcttcggggcagaggacgatgacctgggaggc |
| 201364_s_at | acatcgtccacttccagtatgaggtcaccgaggtgaaggtctcttcttgggatgcagtcctgtccagccagagcctgtttgt |
| SEQ ID | agaaatcccagatggattattagctgatgggagcaaagaaggattgttagcactgctagagtttgctgaagagaagatgaaa |
| NO: 36 | gtgaactatgtcttcatctgcttcaggaagggccgagaagacagagctccactcctgaagaccttcagcttcttgggctttg |
| | agattgtacgtccaggccatccctgtgtcccctctcggccagatgtgatgttcatggtttatcccctggaccagaacttgtc |
| | cgatgaggactaatagtcatagaggatgctttacccaagagccacagtgggggaagaggggaagttaggcagccctgggaca |
| | gacgagagggctcctcgctgtctagggaaggacactgaggggctcagggtgagggttgcctattgtgttctcgg |
| 201870_at | tgtcctgcccaagtgggctgcagactgagtgctgcccttgtagcttccccagaccccaactcactgcagttcatctgaacaa |
| SEQ ID | cctgagctcctgggccggggtggaaggagggggataaacctaaggccctgatccaaagcagcctgttgagctggttctccag |
| NO: 37 | ggctgcagtctctccaggtgtacagctgtccctgccctgtcctgtccttgcacagtctcctatgtctgagccccagtgcctt |
| | ctgttcgggccctcctttggtgggaaaggcagagccctgacccttgaatggttgtccttgactctgtgctgctgccttctgc |
| | agagaggcacctaagctgtttaaagagcccagtgattgtggctgctcctcctagaggtgggagggggcaagaggcctccttg |
| | gtcagtgtccatgctttctgggcagggacttggttttttgttccaacagtggccttctccgggcttcatagttctttgtaa |
| 201909_at | gacaggtgaacatttccgcctggtctatgacaccaagggccgttttgctgttcaccgcatcacagtggaagaggcaaagtac |
| SEQ ID | aagttgtgcaaagtgaggaagattactgtgggagtgaagggaatccctcacctggtgactcatgatgctcgaaccatccgct |
| NO: 38 | acccagatcctgtcatcaaggtgaacgatactgtgcagattgatttagggactggcaagataatcaactttatcaaatttga |
| | tacaggcaatttgtgtatggtgattggtggagccaacctcggtcgtgttggtgtgatcaccaacagggaaagacatcctggt |
| | tcttttgatgtggtgcatgtgaaggatgccaatggcaacagctttgccacgaggctttccaacatttttgtcattggcaatg |
| | gcaataaaccttggatttccctgcccaggggaaagggcattcgacttactgttgctgaagagagagataagaggctggccac |
| | caaaca |
| 201938_at | aaactgtagtttgcctccaagacaccattgtctccctttaatcttctcttttgtatacatttgttacccatggtgttctttg |
| SEQ ID | ttccttttcataagctaataccactgtagggattttgttttgaacgcatattgacagcacgctttacttagtagccggttcc |
| NO: 39 | catttgccatacaatgtaggttctgcttaatgtaacttcttttttgcttaagcatttgcatgactattagtgcttcaaagtc |
| | aatttttaaaaatgcacaagttataaatacagaagaaagagcaacccaccaaacctaacaaggacccccgaacactttcata |
| | ctaagactgtaagtagatctcagttctgcgtttattgtaagttgataaaaacatctggaagaaaatgactaaaactgtttgc |
| | atctttgtatgta |
| 201981_at | acagattgttctcaagagggccatcagaaggaagccaaagagttcacagcctcagcaccaacaactcaacatggtcatcatg |
| SEQ ID | ttttctatatggtttttccagctagcagtactcccttccatacctgtgactgggcagtgcttttctctctcccatgtctagc |
| NO: 40 | ctccaaaagttaagtgaaaattagtcaactgcacgtggaagnnccccaccactttggggatctctttatttcttttcagcca |
| | gggacctgtccactccctttgaattaatatgggaagaaattaatacaggatgaactggagagaagggttgagtgtggcatac |
| | tttctgaaacctggagctgggaattgcggagaagggaaggtctagactagttacatcacatag |
| 202290_at | agggctcatcgacatcgagaaccccaaccgggtggcacagacaaccaaaaaggtcacacaactggatctggacgggccaaag |
| SEQ ID | gagctttcgaggagagaacgagaagagattgagaagcagaaggcaaaagagcgttacatgaaaatgcacttggccgggaaga |
| NO: 41 | cagagcaagccaaggctgacctggcccggctggccatcatccggaaacagcgggaggaggctgcccggaagaaggaagagga |
| | aaggaaagcaaaagacgatgccacattgtcaggaaaacgaatgcagtcactctccctgaataagtaactgcgacccgtggga |
| | ggagatgccggggacctgggccgcgctgccaggacctctgctgtgtctcgcccaccctgtgccctggcgccgctgcaacagc |
| | ccctcatggccaggagccccccatgcctgggcctcctcttcatcttggcacagaaattgtttgggggatgggggggggactg |
| | ggggaggggtagctgctatctttgagacag |
| 202578_s_at | ctggtcacagtggtagtcgctggccccaggaccccctcctgattttggctaggcatcgtggaaccagctccagcccctgaag |
| SEQ ID | aaacgatagatgtgcaggttgtgcggaagaggctgagtggaaaatggtgtgagccccaccgctgtgcatcgaatgagggaag |
| NO: 42 | tggcagcagagaggccagagctgccccctctctgtctcttcaatggacccttcacaagtgtttctcctgcccttggggctgt |
| | cttccttctcctgaaatccagagactttcttgctcatcccttctccctttcacttctgttctctgctgcagaaagcagactt |
| | gcatatccctgatgagcagctcggctacttttgtaaactt |
| 202580_x_at | tcaattgacttctgttccttgcttttagttttgatagaagggaagacctgcagtgcacggtttcttccaggctgaggtacct |
| SEQ ID | ggatcttgggttcttcactgcagggacccagacaagtggatctgcttgccagagtcctttttgcccctccctgccacctccc |
| NO: 43 | cgtgtttccaagtcagctttcctgcaagaagaaatcctggttaaaaaagtcttttgtattgggtcaggagttgaatttgggg |
| | tgggaggatggatgcaactgaagcagagtgtgggtgcccagatgtgcgctattagatgtttctctgataatgtccccaatca |
| | taccagggagactggcattgacgagaactcaggtggaggcttgagaaggccgaaagggcccctgacctgcctggcttcctta |
| | gcttgcccctcagctttgcaaagagccaccctaggccccagctgaccgcatgggtgtgagcc |
| 202727_s_at | aagcgtaaagaggatgtgtggcattttcacttttggcttgtaaagtacagacttttttttttttttaaacaaaaaaagcatt |
| SEQ ID | gtaacttatgaacctttacatccagataggttaccagtaacggaacatatccagtactcctggttcctaggtgagcaggtga |
| NO: 44 | tgccccagggacctttgtagccacttcactttttttcttttctctgccttggtatagcatatgtgttttgtaagtttatgca |
| | tacagtaatttt |
| 202948_at | ggcagctggaatttaaggagggacaagaatcaatggataagcgtgggtggaggaagatccaaacagaaaagtgcaaagttat |
| SEQ ID | tccccatcttccaagggttgaattctggaggaagaagacacattcctagttccccgtgaacttcctttgacttattgtcccc |
| NO: 45 | actaaaacaaaacaaaaaacttttaatgccttccacattaattagattttcttgcagtttttttatggcatttttttaaaga |
| | tgccctaagtgttgaagaagagtttgcaaatgcaacaaaaatatttaattaccggttgttaaaactggtttagcacaattta |
| | tattttccctctcttgcctttcttatttgcaataaaaggtattgagccattttttaaatgacatttttgataaattatgttt |
| | gtactagttgatgaaggagttttttttaacctgtttatataattttgcagcagaagccaaattttttgtatattaaagcacc |
| | aaattcatgtacagcatgcatcacggatcaatagactgtact |
| 203071_at | gcccctggagtcgcggagaaagggccgtaaccggaggacccacgcccctgagcctcgcgctgagcgggggccgcgcagcgca |
| SEQ ID | acgcactggtgaccagactgtccccacgccgggaaccaagcaggagacgacaggcgagagaggagccagacagaccctgaaa |
| NO: 46 | agaaggacgggttggggccgggcacattgggggtcaccggccgatggagacaccaaccgacaggccctggctgagggcagct |
| | gcgcgggcttatttattaacaggataacccttgaatgtagcagccccgggagggcggcacaggtcgggcgcaggattcagcc |
| | ggagggaagggacggggaagccgagctccagagcaacgaccagggccgaggaggtgcctggagtgcccaccctgggagacag |
| | accccacctccttgggtagtgagcagtg |
| 203577_at | attaccgactgtatgcctacacggagtcggagctgcagattgccctcattgccctcttctctgagatgctctatcggttccc |
| SEQ ID | caacatggtggtggcgcaggtgacccgggagagtgtgcagcaggcaatcgccagtggcatcacagcccagcagataatccat |
| NO: 47 | ttcctaaggacaagagcccacccagtgatgctcaaacagacacctgtgctgccccccaccatcaccgaccagatccggctct |
| | gggagctggaaagggacagactccggttcactgagggtgtcctgtataaccagttcctgtcgcaagtggactttgagctgct |
| | gctggcccacgcgcgggagctgggcgtgctcgtgttcgagaactcggccaagcggctcatggtggtgaccccggccgggcac |
| | agcgacgtcaagcgcttttggaagcggcagaaacatagctcctgagagcgcgggacttggac |
| 203642_s_at | cacttagtgtgaaagacttgccttgtgtattctttatgtaattacaaatcactgtcaattttatgggaagctcatagtattt |
| SEQ ID | taatattttattaacatggaactcttgtttttttaatctttagaacttaaattctacaagaattttaaatattttctgtata |
| NO: 48 | taattatgacattgtcacacagaaattacacattttatgtgccagaagccttaaacatctttctgtgaaaatgctgatatat |
| | tgtgacagttatttcacatttgatatgtagagaggaataggggttagtttatgtttatattgaaaaactttaaagactattt |
| | ggaagttccagaaattctggttttaattcaagtaaaatgataaaatagtcattatatagttcagatgctaatattctaagta |
| | ataatatatatttacattgaagctaaaactgttaagcaaaacaatgcccatttgtcggcttacagctcttccggagtctaga |
| | gcctgttggtgttctgtccctactttaagaatttaattgctcacttattctgaa |
| 203679_at | gcgcttcttccaggacaagcgcccggtgcccacgtagcccctgccatggaaggaagaacgggacaaaggaggggcagcaggg |
| SEQ ID | tgtgtgcatatgagacttgggggtccctccccaattttagtttcctgccaaaacgggagtgtgcagtcagggcctgcggtct |
| NO: 49 | ggccccatgagtctccttccgtcctcagcgggcagggaacacctctggcttgtagaagggacggctcagtggctgcaccgac |
| | ggtcctggaaatctcacatggtgggcactgcagcgttggaacgtgagcctcggatttcctggcccctctactgtaaatgtgc |
| | cttagcctaagcctcccatcctgtgttagcgttgcctggtgcggggcagggcctaacaaggaaacctgggccctccaagcca |
| | ggttgaggtctggtaacagaatgccaggaagggggcctggaagaccacctgccccggcccctctcctgcaggggccccacac |
| | aggcatgagggatggcccggccaaagtctaggcagaa |
| 203756_at | tggggacctgtcaggctttgtcatttcccagtttgttggtggtgcctttagtggttccctaatttgggaacactgatggggc |
| SEQ ID | cttggacagggctttctctcaggtaggagaaatgggcccatgatctcctcacagtcgcccccagtccttggccctgcttccc |
| NO: 50 | tgtgtctcatgcactggcacatatggtcaccttggagggcagacctaggagcccctctgaccactgaatccgtctccacacc |
| | ccttctgccaagggaagccccttcaggaaggaccccccaaagctgaggggctgaatgtagccttttcaacagagaaggctcc |
| | cacttgagagcagcctctacctgaccccctggaccacagagagccactctgaccctcagccccctcgcttcttcagctaaaa |
| | ctccaaaggtttggtttcagatggggtttgttttgttctgtttggttttggttttgtttggggtgggtgggtcattgcggtc |
| | ttagattat |
| 203793_x_at | taggcatgattccaactggctgcatataggagtcagttagaattgtttctttctctccccgtttctctccccatcttggctg |
| SEQ ID | ctgtcctgcctctgaccagtggccgccccccgcgttgttgaatgtccagaaattgctaagaacagtgccttttacaaatgca |
| NO: 51 | gtttatccctggttctgaggagcaagtgcagggtggaggtggcacctgcatcacctcctcctcttgcagtggaaactttgtg |
| | caaagaatagatagttctgcctcttttttttttttttcctgtgtgtgtggcctttgcatcatttatcttgtggaaaagaaga |
| | ttcaggccctgagaggtctcagctcttggaggagggctaaggctttagcattgtgaagcgctgcacccccaccaaccttacc |
| | ctcaccggggaaccctcactagcaggactggtggtggagtctcacctggggcctagagtggaagtgggggtgggttaacctc |
| | acacaagcacagatcccagactttgccagaggcaaacagggaattccgccgata |
| 203967_at | taatatctttgggtcttactatttttacccataaaagtgaccaggtagaccctttttaattacattcactacttctaccact |
| SEQ ID | tgtgtatctctagccaatgtgcttgcaagtgtacagatctgtgtagaggaatgtgtgtatatttacctcttcgtttgctcaa |
| NO: 52 | acatgagtgggtatttttttgtttgttttttttgttgttgttgtttttgaggcgcgtctcaccctgttgcccaggctggagt |
| | gcaatggcgcgttctctgctcactacagcacccgcttcccaggttgaagtgattctcttgcctcagcctcccgagtagctgg |
| | gattacaggtgcccaccaccgcgcccagctaattttttaatttttagtagagacagggttttaccatgttggccaggctggt |
| | cttgaactcctgaccctcaagtgatctgcccaccttggcctccctaagtgctgggattataggcgtgagccaccatgctcag |
| | ccattaaggtattttg |
| 203992_s_at | aggaaataacccagttctgcaccactggtttttgtagctatctcgtaaggctgctggctgaaaactgtgtctatgcaacctt |
| SEQ ID | ccaagtgcggagtgtcaaccaactggacgggagagagtactgctcctactccaggactctcacaaagctgatgagctgtact |
| NO: 53 | tcagaaaaaaataataatttccatgttttgtatatatctgacaaaactggcaacatcttacagactactgacttgaagacaa |
| | cctcttttatatttctctatttctgggctgatgaatttgttttcatctgtcttttcccccttcagaattttccttggaaaaa |
| | aaatactagcctagctggtcatttctttgtaaggtagttagcaattttaagtctttctttggtcaacttttttttaatgtga |
| | aaagttaggtaagacacttttttactgcttttatgtttttctgtcttgttttgagaccatgatggttacacttttggttcct |
| | a |
| 204078_at | aacttaagatggacacagctgactggacccccatcctgcctcacccatgggtgctgcaccccagacccatcctgccacttct |
| SEQ ID | atgtctctggaccacaggatggtggtggcattgcaggttggcaagtgggctgatggggtccgccctcctcactgctgagctc |
| NO: 54 | ctcacctggacagtctcctggacaaggagtttccagctgctggctggagtctcaggccaaattgcagagggtcctccagggt |
| | cctgaagagcactggactaagagtctagtggttccagggc |
| 204233_s_at | gcaaacatccggaagtatcccaccaagaaacaacagctccattttatttccagttacttgcctgcattccaaaatgactttg |
| SEQ ID | aaaacctcagtactgaagaaaaatccattataaaagaagaaatgttgcttgaagttaataggtttgcccttgcatctcattt |
| NO: 55 | cctctggggactgtggtccattgtacaagccaagatttcatctattgaatttgggtacatggactacgcccaagcaaggttt |
| | gatgcctatttccaccagaagaggaagcttggggtgtgactgtggggaggactccatccacctcatcactggactgcatggg |
| | gaggcagcagagcggggtcccctctgtgcttcgactactgctcctgtggcaggaggctttgggtggctcactactgaacaca |
| 204409_s_at | aggttatgccatatcagagggaaattgagaaaaaaggtttggataaatacatcagacattatattggttggtctacgggact |
| SEQ ID | atcaggataacaaagctgatgtaattttaaagtacaatgcagatgaagctagaagcctgaaggcatatggcgagcttccaga |
| NO: 56 | acatgctaaaatcaatgaaacagacacatttggtcctggagatgatgatgaaatccagtttgacgatattggagatgatgat |
| | gaagacattgatgatatctaaattgaaccaagtgtttttacatgacaagttctctgaggatggttctacagttgggattttg |
| | gccatcatcaaccaagaagagaaattcatttagtgtgtagtttctgaaagcaaactgatttattttcattgttttaaagtat |
| | ttatttctttaaaagctgaggacactgaattaccttaagttaaatgttaatactttattgttttgatgtaatggaacttaag |
| | gataaaagaccataatatttgctgttaaaataaataaacgagtgcctttcctactgt |
| 204410_at | cctttcctactgtgataacgtcaagtaattggatattttgaatacatttctgcctgataatcatgctgagttctaataagcc |
| SEQ ID | ctacttccacctaatctgtttacagtcttttggtatgtttcagttacttagatggtctcataaggtttctgatacaatttga |
| NO: 57 | agacagaaatctgcatttagaatcagaaaacatggacatatttttcatatttatctagtcatatgtaattttatgctaacat |
| | tgatagtttataaatccttttcatcctttgtgcctcggttattaaggaaaaaaaaatgtccaacatacagtttttaaagtgt |
| | ggcagttttgagtagtaacttagaatgtataagattaagagttaaagaaaccgaacaataagtggcaaccaattatcttaac |
| | attggaaatactggggtgccattttgttttcaaaagttattcattgtaatccactgttttggctttca |
| 204434_at | aatcttctatggctctctttttaatatatatatatatatatatatatatatatatatatatatatatatatctatctggtag |
| SEQ ID | atcacatgtagatgtacagtgtgtatattaagctgaatttcaaccaactttattactaagaccagcctagcagggcattcat |
| NO: 58 | ctttgatgttcttccttttcacatttcagaatcggcaatgcctggcccactcctgagagctctagttttctagttttccatt |
| | tccagattgcgccagcattacagtaactaactctttctaaccaaccgtgatgccacgtaacttaccctatggacaaaagcag |
| | gaatgactaacacatcctaggagggaaagtccagtgtacctgtcatcacggacgcactgtggtcacagactggaatacgaaa |
| | tactacagccctgctcccaggtgtgcatcttactgg |
| 204724_s_at | caccagcaaggacggccaggacggtgctcccggcgagcctgggcctcccggagatcctgggcttccaggtgccattggggcc |
| SEQ ID | caggggacaccggggatctgcgacacctcagcctgccaaggagccgtgttaggaggggtcggggagaaatcaggctctcgaa |
| NO: 59 | gctcataaaattcaacgtgaggaagcaagtgacaaggacgcccgaagcacagtggacggtcatgaaggagcgggggtgtggc |
| | aggcgggtgacgtccaggagagggagcgcccctggctgcccctcggccgccgactggacgcgtgggccttgccagcgagcac |
| | cctcattgggctgtcgcctgacagcatacctcaaaaggccctagctaataaacctgtaagcccagcatttgagagaaggtag |
| | ggtgtgtatatataaaaggttgtgtacaactccacgaggtgaaaaatattcagtaacttgtttgcatagcatttgtgtaaag |
| | actatgatctcatcccaata |
| 204778_x_at | ccggctatgggctcgagccgagttccttcaacatgcactgcgcgccctttgagcagaacctctccggggtgtgtcccggcga |
| SEQ ID | ctccgccaaggcggcgggcgccaaggagcagagggactcggacttggcggccgagagtaacttccggatctacccctggatg |
| NO: 60 | cgaagctcaggaactgaccgcaaacgaggccgccagacctacacccgctaccagaccctggagctggagaaagaatttcact |
| | acaatcgctacctgacgcggcggcggcgcatcgagatcgcgcacacgctctgcctcacggaaagacagatcaagatttggtt |
| | tcagaaccggcgcatgaagtggaaaaaggagaacaagaccgcgggcccggggaccaccggccaagacagggctgaagcagag |
| | gaggaagaggaagagtgagggatggagaaagggcagaggaagagacatgagaaagggagaggaagagaagcccagctctggg |
| | aactgaatcagg |
| 204897_at | agcagcttattgtttctctgaaagtgtgtgtagttttactttcctaaggaattaccaagaatatcctttaaaatttaaaagg |
| SEQ ID | atggcaagttgcatcagaaagctttattttgagatgtaaaaagattcccaaacgtggttacattagccattcatgtatgtca |
| NO: 61 | gaagtgcagaattggggcacttaatggtcaccttgtaacagttttgtgtaactcccagtgatgctgtacacatatttgaagg |
| | gtctttctcaaagaaatattaagcatgttttgttgctcagtgtttttgtgaattgcttggttgtaattaaattctgagcctg |
| | atattgatatg |
| 204921_at | gcccactgcctaaatgatgcccggccagcaggacctggcctgcagatcccagtgagtcatgagcctcagccccctccagccc |
| SEQ ID | actggggctctcacctccacatgtgggtagaagctttcctgccccctcttcctctagtagccctcagtgtcgaaggtgagct |
| NO: 62 | tgtaggtgcctgccttcatctggtccaggacagtgaccatctgggtctgtgtagctggggagagaatgaggctgcagagatg |
| | gggaccagaagccccccaccccagctttcctgggtctgcatcccagtgggcctcagacactgccctgccacctgtcagactt |
| | gggtgagcagacacagtgaggctgttaggtcctgcagttccagagcagtctagggacaccactgccctgtctttaggaaatc |
| 204947_at | ctggctgggcgtgtaggacggtgagagcacttctgtcttaaaggttttttctgattgaagctttaatggagcgttatttatt |
| SEQ ID | tatcgaggcctctttggtgagcctggggaatcagcaaaaggggaggaggggtgtggggttgataccccaactccctctaccc |
| NO: 63 | ttgagcaagggcaggggtccctgagctgttcttctgccccatactgaaggaactgaggcctgggtgatttatttattgggaa |
| | agtgagggagggagacagactgactgacagccatgggtggtcagatggtggggtgggccctctccagggggccagttcaggg |
| | cccagctgccccccaggatggatatgagatgggagaggtgagtgggggaccttcactgatgtgggcaggaggggtggtgaag |
| | gcctcccccagcccagaccctgtggtccctcctgcagtgtctgaagcgcctgcctccccactgctctgccccaccctccaat |
| | ctgcactttgatttgcttcctaacagctctgtt |
| 205000_at | taagcagacccggcattggcaatgtagctgtaattttctgacaaaatttaagacaaaattgtcaacttgaaactaaaacatg |
| SEQ ID | ccaaggttttgatatacttgtcttaagatattaatgaaacaattttgaacactgataggaaggtccacatccacaaagtttc |
| NO: 64 | tcttgagttttgttatgtgttttgctgtgtttgattttcagtgattgtctggtatatttacagtcctcaaacatggttattt |
| | ctgtcagtgacttaacattcggttttaccagccagcagtattcttcagtaaataaagaatggaattgctgaatgtaatcatt |
| | gaacctcgagtcactgtaaaagttcagtaattgcttattgtattagttttagatgctggcaccgcatgtgctctgtttattc |
| 205001_s_at | agaagtgccttcttggttggaaaatatggcttatgaacaccactacaagggtggcagtcgtggacgatctaaaagtaataga |
| SEQ ID | ttcagtggaggatttggtgccagagactatcgacaaagtagtggttccagcagttccggctttggtgctagtcgcggaagca |
| NO: 65 | gcagccgcagtggtggaggtggttacggcgacagcagaggatttggtggaggtggctatggaggcttctacaatagtgatgg |
| | atatggaggaaattataactcccagggggttgactggtggggcaactgaatctgctttgcagcaaagtcacccttacaaaga |
| | agctaatatggaaaccacatgtaacttagccagactatattgtgtagcttcaagaacttgcagtacattaccagctgtgatt |
| | ctcctgataattcaagggagctcaaagtcacaagaagaaaaatgaaaggaaaaaacagcagccctattcagaaattggtttg |
| | aagatgtaattgctctagtttggatt |
| 205027_s_at | attatataactctttgagcctttattggtaaattctggtatacattgaattcattataatttgggtgactagaacaacttga |
| SEQ ID | agattgtagcaataagctggactagtgtcctaaaaatggctaactgatgaattagaagccatctgacagacggccactagtg |
| NO: 66 | acagtttcttttgtgttcctatggaaacattttatactgtacatgctatgctgaagacattcaaaacgtgatgttttgaatg |
| | tggataaaactgtgtaaaccacataattttgtacatccaaggatgaggtgtgacctttaagaaaaatgaaaacttttgtaaa |
| | ttattgatgattttgtaattcttatgactaaattttcttttaagcatttgtatattaaaatagcatactgtgtatgttttat |
| | atcaaatgccttcatgaatctttcatacatatatatatttgtaacatgtaaagtatgtgagtagtcttatgtaaagtatgtt |
| | tttacattatgcaaataaaacccaatacttttgtccaatgtggttggtcaa |
| 205105_at | acttcttctctcatgaatcatccagtcattccaatggcaaataagttctcacctacccttgagctgcaaggtgaattctctc |
| SEQ ID | cattacagtcatctttgccttgtgacattcatctggttaatttgagaacaatacagtcaaaggtgggcaatgggcactccaa |
| NO: 67 | tgaggcagccttgatcctccacagaaaagggtttgattgtcggttctctagcaaaggcacagggctgttttgttctactact |
| | cagggaaagatattggtacagaaacttttaaacaagtttattgtcgaaagtctcacaccttcatcactatccttgatgcatt |
| | cacctcccggcactcagaatataagtgagatcaacttgagtccaatggaaatcagcacattccgaatccagttgaggtgaac |
| | ctgactttcacatttggattgagaatcattggcttttatacctttcttggt |
| 205204_at | agatccgagtgcactcgcgaggcaacctctgggccaccggtcacttcatgggcaagaagagtctggagccttccagcccatc |
| SEQ ID | ccattggggacagctccccacacctcccctgagggaccagcgactgcagctgagtcatgatctgctcggaatcctcctgcta |
| NO: 68 | aagaaggctctgggcgtgagcctcagccgccccgcaccccaaatccagtacaggaggctgctggtacaaatactgcagaaat |
| | gacaccaataataggggcagacacaacagcgtggcttagattgtgcccacccagggaaggtgctgaatgggaccctgttgat |
| | ggccccatctggatgtaaatcctgagctcaaatctctgttactccattactgtga |
| 205419_at | cagcccctgaagaaaattcacgtgaaatgacagaaacgcagatgatgatacattccaagtcttcaaatggaaagtgaaatgg |
| SEQ ID | attgtattttggtttatagtgacgtaaactgtatgacaaactttgcaggacttcccttataaagcaaaataattgttcagct |
| NO: 69 | tccaattagtattcttttatatttctttcattgggcgctttcccatctccaactcggaagtaagcccaagagaacaacataa |
| | agcaaacaacataaagcacaataaaaatgcaaataaatattttcatttttatttgtaaacgaatacaccaaaaggaggcgct |
| | cttaataactcccaatgtaaaaagttttgttttaataaaaaattaattattattcttgccaacaaatggctagaaaggactg |
| | aatagattatatattgccagatgttaatactgtaacatactttttaaataacatatttcttaaatccaaatttctctcaatg |
| | ttagatttaattccctcaataacaccaa |
| 205421_at | caattcctattcattcttagcactttgacatgtcttggggaaaagctttacattttaatttaaaagaaagatcaattatatc |
| SEQ ID | catgcttaacaggatcagcaggagctttataaatgactttacagagactaataagggattgatctttctttttttgttatcg |
| NO: 70 | aggcttttgaaatgtggaacttgtgtgttctgctttatatgttatattcaatatcttttcagatgcagtctatattttatgc |
| | tgagttttaaaaatgaaatactttatgcaaacaggcaaaattggtaccaaagggaaacattaaccatgaggaagagcatttt |
| | tctaaggagaacaggtgacaatatacacatgtcgcgtaatcgtaaaatgagcatcttagtctttaaaacaca |
| 205447_s_at | gccgtcacccgaagtcagaaacgtggcatctcatcggaagaggaggaaggagaggtagacagtgaagtagagctgacatcaa |
| SEQ ID | gccagaggtggcctcagagcctgaacatgcgccagtcactatctaccttcagctcagagaatccatcagatggggaggaagg |
| NO: 71 | cacagctagtgaaccttcccccagtggcacacctgaagttggcagcaccaacactgatgagcggccagatgagcggtctgat |
| | gacatgtgctcccagggctcagaaatcccactggacccacctccttcagaggtcatccctggccctgaacccagctccctgc |
| | ccattccacaccaggaacttctcagagagcggggccctcccaattctgaggactcagactgtgacagcactgaattggacaa |
| | ctccaacagcgttgatgccttgcggcccccagcttccctccctccatgaaagccactcgtattccttgtac |
| 205518_s_at | tcaagaattcactgatctgatgcaaaataaaaatttatcattacatcttgaacccaggaagcttacagcaaagagactatgc |
| SEQ ID | tttatgacgtcagcaatagataattccacgttgcctttgtgatttgtatatatagcttacatttgtggaccactacatagcc |
| NO: 72 | agattcaaaaatattttacttgttccatccacagttctctacagaaagaaccaatgaacccaataggaacaaattctctgtg |
| | gaaaacaaagcatagctgtagtagatacgaatccaatcacagaggaaacaggaagagaaaaacatccaagactacagtgaaa |
| | actggaaatggtctgttttcgtgatattcgtatgattaagatgcaaattttttcttaggaaaatgtgattgttaactagcat |
| | tctgttttacatgttgacatttctaacacacacaccactgatttgaacttcaaaatttattttctgattatatatgctaggt |
| | ctgattctgaag |
| 205668_at | ggaactctaaaccttgtgatgactactaacaaatgtaaaattatgagtgattaagaaaacattgctttgtggttatcacttt |
| SEQ ID | aagttttgacacctagattatagtcttagtaatagcatccactggaaaaggtgaaaatgttttattcagcatttaacttaca |
| NO: 73 | tttgtactttagagtatttttgtataaaatccatagatttattttacatttagagtatttacactatgataaagttgtaaat |
| | aattttctaagacagtttttatatagtctacagttgtcctgatttcttattgaatttgttagactagttctcttgtcttgtg |
| | atctgtgtacaattttagtcactaagactttcctccaagaactaagccaacttgatgtgaaaagcacggctgtatataatgg |
| | tgatgtca |
| 205841_at | atctattttattatggtttcccttgtatctatttgtggtgaatgtgttttttaaatggaactatctccaaatttttctaaga |
| SEQ ID | ctactatgaacagttttcttttaaaattttgagattaagaatgccaggaatattgtcatcctttgagctgctgactgccaat |
| NO: 74 | aacattcttcgatctctgggatttatgctcatgaactaaatttaagcttaagccataaaatagattagattgttttttaaaa |
| | atggatagctcattaagaagtgcagcaggttaagaattttttcctaaagactgtatatttgaggggtttcagaattttgcat |
| | tgcagtcatagaagagatttatttcctttttagaggggaaatgaggtaaataagtaaaaaagtatgcttgttaattttattc |
| | aagaatgccagtagaaaattcataacgtgtatctttaa |
| 205856_at | gaaatactcagcttaggcatttttactttaacccctaaattgattttgtaaatgccacaaatgcatagaattgttaccaacc |
| SEQ ID | tccaaagggctctttaaaatcatatttttttattcatttgaggatgtcttataaagactgaaggcaaaggtcagaatgctta |
| NO: 75 | cgggtgttatttttataagttgttgaattccttaatttagaaaagctcattattttttgcacactcacaatattctctctca |
| | gaaatcaatggcatttgaaccaccaaaaa |
| 205987_at | ccagctgttgctggtttgtcatgcctccggcttctacccaaagcctgtttgggtgacatggatgcggaatgaacaggagcaa |
| SEQ ID | ctgggcactaaacatggtgatattcttcctaatgctgatgggacatggtatcttcaggtgatcctggaggtggcatctgagg |
| NO: 76 | agcctgctggcctgtcttgtcgagtgagacacagcagtctaggaggccaggacatcatcctctactggggacaccactcttc |
| | catgaattggattgccttggtagtgatagtgcccttggtgattctaatagtccttgtgttatggtttaagaagcactgctca |
| | tatcaggacatcctgtgagactcttccccctgactcccccattgtgttaagaacccagcaacccaggagcctagtacaatat |
| | agtgatgccatcccgtcgactctccatttaaattgtt |
| 205997_at | gatggtaaaggctgttcaaccccaagagatgagtcagatgaagccccatgtgtatgatctgccagtagaaggcaatgagccc |
| SEQ ID | ccagcctcttttcataaagacacaaacgcacttccccctactgttttcaaggataatccaatgtctacacctaaggactcaa |
| NO: 77 | atccagaagcatgaagcaacagctaagcaagaactaatggttaaattatcaacttggaaaacttggaaaatctggatgccag |
| | agaaatatactatctcaccagtatttgctctcgactcaagaaggttaacattttctgattcatgttagactttgaagagact |
| | aaagaaaattttcaagaggaacatatgcctgagaacctttgca |
| 206118_at | gctgacatcctgcgagactacaaagttattatggctgaaaacattcctgaaaaccctctgaagtacctatatcctgacattc |
| SEQ ID | ccaaagacaaagccttcggtaaacactacagctctcagccttgcgaagtttcaagaccaacagaaaggggtgacaaaggtta |
| NO: 78 | tgttccttctgtttttatccccatctcaacaatccgaagtgattcaacagagccacattctccatcagaccttcttcccatg |
| | tctccaagtgtgtatgcggtgttgagagaaaacctgagtcccacaacaattgaaactgcaatgaagtctccttattctgctg |
| | aatgacaggataaactctgacgcaccaagaaaggaagcaaatgaaaaagtttaaagactgttctttgcccaataaccacatt |
| | ttatttcttcagctttgtaaataccaggttctaggaaatgtttgacatctgaagctctcttcacactcccgtggcactcctc |
| | aattgggag |
| 206618_at | gaagcatatccagagggcgaaagatatctctccattgtgcatctgcctcttttgacgttggaagacacatgtcttactcccc |
| SEQ ID | aaagggagcccagcactgggagccttcttgatgatctcaaaaataatagctattcaagaaaatcaccaagtgactgtgaaac |
| NO: 79 | cgtcagttcggaaggctggttagaacatgtgggagcaacatgaatgttctacaaaagtttaaagcagagattgtttcaaatg |
| | ggtgtagtagatattactgaaaaccaaaaaagagtgagattgtcagtgtaagaatgtgatttaatgtttgtagtgcttacaa |
| | ttttgtgtaccaactggatgactaaaaagagtaaaataacttaattaatagctcatattttatgtgtgaaaacatgttagtg |
| | aacatatataatcaaaatagatttcattgctattgcatagtctc |
| 206637_at | tgagcctggggttctggtgttagaatatttttaagtaggctttactgagagaaactaaatattggcatacgttatcagcaac |
| SEQ ID | ttcccctgttcaatagtatgggaaaaataagatgactgggaaaaagacacacccacaccgtagaacatatattaatctactg |
| NO: 80 | gcgaatgggaaaggagaccattttcttagaaagcaaataaacttgatttttttaaatctaaaatttacattaatgagtgcaa |
| | aataacacataaaatgaaaattcacacatcacatttttctggaaaacagacggattttacttctggagacatggcatacggt |
| | tactgacttatgagctaccaaaactaaattctttctctgctattaactggctagaagacattcatctatttttcaaatgttc |
| | tttcaaaacatttttataagtaatgtttgtatctatttcatgctttact |
| 206700_s_at | tcttaaatcctactatctcctggcctggacctcagaaggagctttttgcctatctataatttttcactgccaatttttgata |
| SEQ ID | tcctctctcctagagttactgttaaaaggttggttcgtaaagtccacaccccgatgctcagaagtgtcttgccagcaacatt |
| NO: 81 | cctgctagcatacaggagtgatttcctaaaccagtttcattctagtctgaatagggacaaacaaatcttgaggaagcccaag |
| | tgcgtacctttatttttgcccccaccaccctctttctgtacttcaatttttgtttgttttttgtttttttgtccctgtcata |
| | aaatattttggtgcttcaaaacttgtaccttcattgtacatccttttcttttctccccttgggtcttattataaaagaagac |
| | aatgtacgttgtaattaccaaaaagaatagggaaaaacaagaatttcatgactctacctgtggtctatcttt |
| 207063_at | tgttgggaattggtactggctagaaatttctgttgagtatttattaccccatggtaataatggtaaaccacagtttagaaag |
| SEQ ID | attttttttgacagccacagcatgttccgaagagatgattggaagatggaagtggagggttaaataatgaaatgcagctaac |
| NO: 82 | atttcggaaagtttctaaaagttgtacaacatgccctacagctactctttaaatctccaaatcaaatgagtttcaggtggag |
| | cctctgggaggtgatgaggtcatgagagtggagcctcatgaatgggatgagcactcctacaaaaaggattccagagagctcg |
| | cttgctccttccacagtgtgaggacacagagggaaggctctgtctatgaatgagaaagtgggtccccaccagacattgaatc |
| | tgccgcatcttgatactggacttccagtctccagaactgtgggcaataaatgtctgttgtttattacctgtccagtatcttt |
| | ggtattttgctatagcaacccaaatggactaagaaaacaccagaggccatacctaat |
| 207651_at | ttgccttgtaattcgacagctctacagaaacaaagataatgaaaattacccaaatgtgaaaaaggctctcatcaacatactt |
| SEQ ID | ttagtgaccacgggctacatcatatgctttgttccttaccacattgtccgaatcccgtataccctcagccagacagaagtca |
| NO: 83 | taactgattgctcaaccaggatttcactcttcaaagccaaagaggctacactgctcctggctgtgtcgaacctgtgctttga |
| | tcctatcctgtactatcacctctcaaaagcattccgctcaaaggtcactgagacttttgcctcacctaaagagaccaaggct |
| | cagaaagaaaaattaagatgtgaaaataatgcataaaagacaggattttttgtgctaccaattctggccttactgga |
| 207714_s_at | gacacatgggtgctattggggttgggggggaggtgaggtaccagccttggatactccatggaattcgagctccacttggaca |
| SEQ ID | tgggccccagataccatgatgctgagcccggaaactccacatcctgtgggacctgggccatagtcattctgcctgccctgaa |
| NO: 84 | agtcccagatcaagcctgcctcaatcagtattcatatttatagccaggtaccttctcacctgtgagaccaaatt |
| 208296_x_at | ttgagttctccttttaagtaccaatgatacttaaatttctcagaaatgtaatggtgtgtcattgccttgaaatgcttgctta |
| SEQ ID | gggcttcttttatgttatcttaaaaagtgctggtgaattttccattttttacatccatttcacatgtaagagacacaaaagt |
| NO: 85 | ctagattggtcttgatattgagataataaaaagtaagtagcattaagaaaggtaacaatcttcattctacagatgaactcat |
| | tgaaacaatttaggggaatgaggggcaaaaggggagaaatactgctaaagaacatgagcataaaaatgcgtgcgtttcagtg |
| | tttaagaaggcttgataaagaatgtcacttttttatttaactgataagatttttgttattttttactttgataagtaaacca |
| | aagaatatttgtatttcaagcagtttgtgtggtgtttctatataattttctgtgtataaataataaagtaggcatttgttta |
| | ttttgtaaaaaagaaatgaaaatctgctggccagctatgtcctctaggaaatgacagacccaaccacca |
| 208498_s_at | taaccatgacaatcaacgaggacatggcgctggaggagcctctatacttaccttctgggatgctaggctgtacaaaatggca |
| SEQ ID | gttggatttatgcttgctcatccttatggatttacacgagtaatgtcaagctaccgttggccaagatattttgaaaatggaa |
| NO: 86 | angatgttaatgattgggttgggccaccaaatgataatggagtaactaaagaagttactattaatccagacactacttgtgg |
| | caatgactgggtctgtgaacatcgatggcgccaaataaggaacatggttaatttccgcaatgtagtggatggccagcctttt |
| | acaaactggtatgataatgggagcaaccaagtggcttttgggagaggaaacagaggattcattgttttcaacaatgatgact |
| | ggacattttctttaactttgcaaactggtcttcctgctggcacatactgtgatgtcatttctggagataaaattaatggcaa |
| | ctgcacaggcattaaaatctacgtttctgatgatggcaaagctcatttttctattagtaactctgctgaagatccatttatt |
| | gcaattcatgctgaatctaa |
| 208688_x_at | tgagctacaggactcccgagtgtgagccgcggttcctctgttgcagcgcagccgtgtgtgctgtggagccgaggccgtcctg |
| SEQ ID | caggaagccgcgtgactcccgcctcctccctgtgctctctggctctggactgtgactgcgcctggattctgccattgcgaca |
| NO: 87 | catttttgtgcctttcagcccctggtgtctgcagtgggggatttaaggcacccgcttccacttctttcttgtttggagtttt |
| | ctgttggaaccgccggcgttggctccgaagacttagcgacgccactggcggcaccttctcctgcgcccagtgatgtttccac |
| | ggtgcctgtacacagccgagcagcatttccgttgaaggacttgcatccccattgcgggcagtgctggacgtgtcccggagac |
| | ccaccgggaggcgccgcatgccttgtacccccaccgtgcaggttgtggccggtttt |
| 208799_at | ctggcctctactacgtggacagtgaagggaaccggatttcaggggccaccttctctgtaggttctggctctgtgtatgcata |
| SEQ ID | tggggtcatggatcggggctattcctatgacctggaagtggagcaggcctatgatctggcccgtcgagccatctaccaagcc |
| NO: 88 | acctacagagatgcctactcaggaggtgcagtcaacctctaccacgtgcgggaggatggctggatccgagtctccagtgaca |
| | atgtggctgatctacatgagaagtatagtggctctaccccctgaaagagggtggatgcagctgcttgtgtttcttggggtga |
| | ctgtcattggtaatacggacacagtgacccatcctccatcctatttatagtggaagggccttcaattgtatcagtacttttt |
| | tttaagctctggcacattgacctctat |
| 208894_at | cgatcaccaatgtacctccagaggtaactgtgctcacgaacagccctgtggaactgagagagcccaacgtcctcatctgttt |
| SEQ ID | catagacaagttcacccca |
| NO: 89 | |
| 209078_s_at | agcgacttcttctgaggaggttcctggcctctgtcatctccaggaagccctctcagggtcagtggccacccctcacttccaa |
| SEQ ID | agccctgcagaccccacaatgcagtcctggtggcctgactgtaacacccaacccagcccggacaatatacaccacgaggatc |
| NO: 90 | tccttgacaacctttaatatccaggatggacctgactttcaagaccgagtggtcaacagtgagacaccagtggttgtggatt |
| | tccacgcacagtggtgtggaccctgcaagatcctggggccgaggttagagaagatggtggccaagcagcacgggaaggtggt |
| | gatggccaaggtggatattgatgaccacacagacctcgccattgagtatgaggtgtcagcggtgcccactgtgctggccatg |
| | aagaa |
| 209129_at | ggccaccctggagaaatgtgccacgtgctcccagcccatcctggaccggatcctgcgggctatggggaaggcctaccaccct |
| SEQ ID | ggctgcttcacctgcgtggtgtgtcaccgcggcctcgacggcatccccttcacagtggatgctacgagccagatccactgta |
| NO: 91 | ttgaggactttcacaggaagtttgccccaagatgctcagtgtgcggtggggccataatgcctgagccaggtcaggaggagac |
| | tgtgagaattgttgctctggatcgaagttttcacattggctg |
| 209150_s_at | cagttgtctggggaggattaccgctggtggtggcgatctgtgctgagtgttggctccaccggcctcttcatcttcctctact |
| SEQ ID | cagttttctattatgcccggcgctccaacatgtctggggcagtacagacagtagagttcttcggctactccttactcactgg |
| NO: 92 | ttatgtcttcttcctcatgctgggcaccatctcctttttttcttccctaaagttcatccggtatatctatgttaacctcaag |
| | atggactgagttctgtatggcagaactattgctgttctctccctttcttcatgccctgttggactctcctaccagcttctct |
| | tctgaatgactgaattgtgtgatggcattgttgccttccctttgccctttgggcattccttccccagagagggcctggaaat |
| | tataaatctctatcacataaggattatatatttgaactttttaagttgcctttagttttggtcctgat |
| 209254_at | gggtcattgtttaagatctggctggtggtacctagcctgctggaactggcatgggagaagctgcttgcggccttccctaacc |
| SEQ ID | ttgcaaacctctcccgaacacaacttctgcaccttggactcacacagggactcatcgaacgcttgaaatgaggatttntgga |
| NO: 93 | ctgttcattgatactggaaatgttaatttaaagagactcctttatttatgggcagtgtagaatgtgctacaaagaggattgg |
| | ttaccctgatcaaggccttatttagaaaatacatcagatgcctttctgtaaattggtttttcagtttatggacatctcactt |
| | tcccacgtgcttccttctttgcttctgttcctcctgacccattacatgcacatgtactcacatactccctcttccttctcga |
| | tggagttaa |
| 209262_s_at | tcgggccagtgaccatgacggggccacgtgtgctgtggccaggcctgcagacagacctcaagggacagggaatgctgaggcc |
| SEQ ID | tcgaggggcctcccggggcccaggactctggcttctctcctcagacttctattttttaaagactgtgaaatgtttgtctttt |
| NO: 94 | ctgttttttaaatgatcatgaaaccaaaaagagactgatcatccaggcctcagcctcatcctccccaggacccctgtccagg |
| | atggagggtccaatcctaggacagccttgttcctcagcacccctagcatgaacttgtgggatggtggggttggcttccctgg |
| | catgatggacaaaggcctggcgtcggccagaggggctgctccagtgggcaggggtagctagcgtgtgccaggcagatcctct |
| | ggacacgtaacctatgtcagacactac |
| 209277_at | gtgagactgaattcttgcaatgcataagatataaaagcaaatatgactcactcatttcttggggtcgtattcctgatttcag |
| SEQ ID | aagaggatcataactgaaacaacataagacaatataatcatgtgcttttaacatatttgagaataaaaaggactagcaaata |
| NO: 95 | aaactcattttgcatttaaaagttggnattatattttagtcccaagaagacaaagtcgcagattaacaacactttaaaaata |
| | tnactcccattttattgtgttagattgcagtacaatgaatgaataaggggatttatttaaaagatcttaactttcttgactg |
| | atttaaaaagctaatcaatttattaaagccagaaatgttctcctctgggaaagcaatttctatct |
| 209606_at | gaattgcaaaactgacatcccatttcacagcaatagtgacctttatttaaattgttgtgttatagtttatgcttcttaaatc |
| SEQ ID | atttttcaacctaaacagccaatttctaagcagacaggaaaactaaataataagttaattaatataacaaagatgcaggttc |
| NO: 96 | ctgctcattccagtaatgtctttgaaagcaaaactaatatttattttctagattatccctgtgaataattgagaactttttg |
| | gagtcaagtatgaataaaggtgtggcagaatataataatctggactattttctataggataattgctgggttataaaatctt |
| | aggtttgcttatgcccagtagctcctgcggaggcttaataataggcaattttgaatttgttcaaacctgtaatggcttgtaa |
| | acaaagatgaccatcagctgtttctcacatctatagtgacaataaagcgggaagtataagatttaataggaggggttaaggt |
| | tcatgagaaccatggaaagatgtggtctgagatgggtgctgcaaagat |
| 209728_at | gcatccaagcatgatgagccctctcacggtgcaatggagtgcacggtctgaatctgcacagagcaagatgctgagtggagtc |
| SEQ ID | gggggctttgtgctgggcctgctcttccttgggacagggctgttcatctacttcaggaatcagaaaggacactctggacttc |
| NO: 97 | agccaacaggactcttgagctgaagtgcagatgaccacattcaaggaagaaccttctgccccagctttgcaagatgaaaagc |
| | tttcccacttggctcttattcttccacaagagctttgtcaggaccaggttgttactggttcagcaactctgcagaaaatgtc |
| | ctcccttgtggcttccttagctcctgttcttggcctgaagcctcacagctttgatggcagtgcctcatcttcaacttttgtg |
| | cttccctttacctaaactgtcctgcctcccgtgcatctgtactccccttgtgccacacattgcattattaaatg |
| 209795_at | tagtctaattgaatcccttaaactcagggagcatttataaatggcaaatgcttatgaaactaagatttgtaatatttctctc |
| SEQ ID | tttttagagaaatttgccaatttactttgttatttttccccaaaaagaatgggatgatcgtgtatttatttttttacttcct |
| NO: 98 | cagctgtagacaggtccttttcgatggtacatatttctttgcctttataatcttttatacagtgtcttacagagaaaagaca |
| | taagcaaagactatgaggaatatttgcaagacatagaatagtgttggaaaatgtgcaatatgtgatgtggcaaatctctatt |
| | aggaaatattctgtaatcttcagacctagaataatactagtcttataataggtttgtgactttcctaaatcaattctattac |
| | gtgcaatacttcaatacttcat |
| 210017_at | aatgactgcaaaagtgccacggatatcaatttgagggttataaattttagcaagttggtaaattcacaaatacataaccttg |
| SEQ ID | aataatgaggatcaactgtaccatatttaataaagcacaaaacccananananngtcttattacagcatttgataaaatcca |
| NO: 99 | aaactctttcataaaaacactcaacaaacttaggaataaaaggaatcttcctagatatgataaatataacatctatgaaang |
| | cccacacctaacattatacttcatggtgatagactgaaggctgaatgttttccccttaagattgggaagaaggacaaggatg |
| | ttcactcggcactacttctattcagcattgtacttgaagttctagccacagcagttaggttaggaattcaaggtttgttcaa |
| | cat |
| 210052_s_at | agtcaagtgaccagcctctgactgtgcctgtatctcccaaattctccactcgattccactgctaaactcagctgtgagctgc |
| SEQ ID | ggataccgcccggcaatgggacctgctcttaacctcaaacctaggaccgtcttgctttgtcattgggcatggagagaaccca |
| NO: 100 | tttctccagacttttacctacccgtgcctgagaaagcatacttgacaactgtggactccagttttgttgagaattgttttct |
| | tacattactaaggctaataatgagatgtaactcatgaatgtctcgattagactccatgtagttacttcctttaaaccatcag |
| | ccggccttttatatgggtcttcactctgactagaatttagtctctgtgtcagcacagtgtaatctctattgctattgcccc |
| 210258_at | acagcaagcctatgtagttcaattaatatataaggaaaaggaaggtctttcttcatgatacaagcattataaagtttttact |
| SEQ ID | gtagtagtcaattaatggatatttccttgttaataaaattttgtgtcataatttacaaattagttctttaaaaattgttgtt |
| NO: 101 | atatgaattgtgtttctagcatgaatgttctatagagtactctaaataacttgaatttatagacaaatgctactcacagtac |
| | aatcaattgtattataccatgagaaaatcaaaaaggtgttcttcagagacattttatctataaaattttcctactattatgt |
| | tcattaacaaacttctttatcacatgtatcttctacgtgtaaaacatttctgatgattttttaacaaaaaatatatgaattt |
| | cttcatttgctcttgcatctacattgctataanggatataaaatgtggtttctatattttgagatgttttttccttacaatg |
| | tgaactcatcgtgatcttgg |
| 210260_s_at | gattgagtcatcgacattcaggatttaagtctgaggtagtcaaccctcaggaaaaaaaaaatggcttatctgaaatcagtac |
| SEQ ID | tgtggaaatgaactatattagctattatgaataatgtccagtataagaatatgcttctggaattgagttctccttttaagta |
| NO: 102 | ccaatgatacttaaatttctcagaaatgtaatggtgtgtcattgccttgaaatgcttgcttagggcttcttttatgttatct |
| | taaaaagtgctggtgaattttccattttttacatccatttcacatgtaagagacaaaaaagtctagattggtcttgatattg |
| | agataataaaaagtaagtagcattaagaaaggtaacaatcttcattctacagatgaactcattgaaacaatttaggggaatg |
| | aggggcaaaaggggagaaatactgctaaagaacatgagcataaaaacgcgtgcgtttcagtgtttaagaagg |
| 210306_at | aatgggtttaatgaggtctaccttgcagagccattgtgagcattggaaatgatgaatgaatcataccagaacgtctagtata |
| SEQ ID | attacagtcatgcattgcttaacgatggggatacattcttagaaatgtgtcactaggcaattctgtcattgtgtaaacatta |
| NO: 103 | taggatgtacttacacaaacctagatgttatatgtatttttatttacatgtatattttcacatgaaataccaaatgtcacag |
| | cattattactgaatgtcagtcatttcccctacttgatctgcaatgccaatatcaagggccatgtatcaggtttctgtatatg |
| | ttccactataatcttatgggaccatggttttaaatgtggaatcattgacagaaatgtntttatgtagcatatggctgtgtat |
| | cactagtatataatagagcaatattatggaggaatatgtagatccaatcactttacctatacaaaatgactgctatggtggg |
| | aacaca |
| 210976_s_at | gcgtaagagggctctggtcttccaaccagtggctgagctgaaggaccagacagattttgagcatcgaatccccaaggaacag |
| SEQ ID | tggtggctgaaactgaggcccatcctcaaaatcctagccaagtacgagattgacttggacacttcagaccatgcccacctgg |
| NO: 104 | agcacatcacccggaagcggtccggggaagctgccgtctaaacctctctggagtgaggggaatagattacctgatcatggtc |
| | agctcacaccctaataagtccacatcttctcagtgttttagctgtttttttcattaggtttccttttattctgtaccttgca |
| | gccatgaccagttctggccaggagctggaggagcaggcagtgggtgggagctccttttaggtagaatttaacatgacttctg |
| | ccccagctttatctgtcacacaaggctgggcacctctagtgctactgctagatatcacttactcagttagaattttcctaaa |
| | aataagctttatttatttctttgtgataacaaagagtcttggttcctctactactt |
| 211149_at | cagaaacctcgatatataattgtatagattttaaaagttttattttttacatctatggtagtttttgaggtgcctattataa |
| SEQ ID | agtattacggaagtttgctgtttttaaagtaaatgtcttttagtgtgatttattaagttgtagtcaccatagtgatagccca |
| NO: 105 | taaataattgctggaaaattgtattttataacagtagaaaacatatagtcagtgaagtaaatattttaaaggaaacattata |
| | tagatttgataaatgttgtttataattaagagtttcttatggaaaagagattcagaatgataacctcttttagagaacaaat |
| | aagtgacttatttttttaaagctagatgactttgaaatgctatactgtcctgcttgtacaacatggtttggggtgaaggg |
| 211421_s_at | aaggcttggatgcgtgtgtaatagagccttatggtgtgtgcgcacacaccacgagggagagtttgaaaaatgcttattggac |
| SEQ ID | acgtaacctggctctaatttgggctgtttttcagatacactgtgataagttcttttacaaatatctatagacatggtaaact |
| NO: 106 | tttggttttcagatatgcttaatgatagtcttactaaatgcagaaataagaataaactttctcaaattattaaaaatgccta |
| | cacagtaagtgtgaattgctgcaacaggtttgttctcaggagggtaagaactccaggtctaaacagctgacccgagtatggg |
| | gaatttatccgttaccaatttatccttgaccaataacctaattgtctattccgtagttataaaagtccccatccttattagc |
| | tctactggaattttcatacacggtaaatgcagaagttactaagtattaagtattactgagtattaagtagtaatctgtcagt |
| | tattaaaatttgtaaaatctatttatgaaaggtcattaaaccagatcatgttcctttttttgt |
| 211603_s_at | ggtgctgccctgtgtacatataaatgaatctggtgttggggaaaccttcatctgaaacccacagatgtctctggggcagatc |
| SEQ ID | cccactgtcctaccagttgccctagcccagactctgagctgctcaccggagtcattgggaaggaaaagtggagaaatggcaa |
| NO: 107 | gtctagagtctcagaaactcccctgggggtttcacctgggccctggaggaattcagctcagcttcttcctaggtccaagccc |
| | cccacaccttttccccaaccacagagaacaagagtttgttctgttctgggggacagagaaggcgcttcccaacttcatactg |
| | gcaggagggtgaggaggttcactgagctccccagatctcccactgcggggagacagaagcctg |
| 211676_s_at | gaaaatattcctgacgtggtcccgggcagccatctgactccaatagagagagagagttcttcacctttaagtagtaaccagt |
| SEQ ID | ctgaacctggcagcatcgctttaaactcgtatcactccagaaattgttctgagagtgatcactccagaaatggttttgatac |
| NO: 108 | tgattccagctgtctggaatcacatagctccttatctgactcagaatttcccccaaataataaaggtgaaataaaaacagaa |
| | ggacaagagctcataaccgtaataaaagcccccacctcctttggttatgataaaccacatgtgctagtggatctacttgtg |
| 212134_at | tcagccctgatgcacaggctgccagccccccagtccagccctctcccttccactggtgccttgcttagagccagaagggatg |
| SEQ ID | aagccgggggatctatggaacagaggaggagcgatgcagttgggagaggaagctagaagggttatggttggagttctgtana |
| NO: 109 | gtgttgagtttccgacagggaaagaggattcctccaatgcncctagagagaaagcctgagcaggagatgatgcagcagaggg |
| | gaagggccctgtggtgccgccgcccttccttcagcctccgaagggtgatggaaatggagagtggaggaccaggcctccagct |
| | gtctggcctcgcccttcacgccttaacactaagcccacctcccctgctctccttcccagcattgagcccttggttgcctggg |
| | cccaggctgggggttttcagtatttgtaagcatttcagcagaac |
| 212349_at | tggagtgtgttcctgaagagcagccaggaggccagcatggctggagaggcaggcataggcagggaaccgagcagcaggtcag |
| SEQ ID | agcaggcgagctgacattctgcagcctggacggccatggcaggaagcttttagttggagagatacaggaagcctcctagggt |
| NO: 110 | tctgagcagaagaggggcatgagctgattcacattctgaaggacctctctagctggccagtgctgaggaggttggagagaga |
| | aagggtgaaagcagagagaccagtgcagggctgttaacagggttgcaggcgagagactggggtgctgggctcccctagacta |
| | ggactccagtgccctcctctcccaagagacaaaggccattgc |
| 212379_at | aaatcgtggcaaaatcttccctccagtactcctctccagcacctgatggttgtggtgaccagactttaggggacttacttct |
| SEQ ID | cacgcctaccagaatctacagccattcactgttacctgtcctacgttcaggacatgtcaaagcctttgcccatattactggt |
| NO: 111 | ggaggattactagagaacatccccagagtcctccctgagaaacttggggtagatttagatgcccagacctggaggatcccca |
| | gggtcttctcatggttgcagcaggaaggacacctctctgaggaagagatggccagaacatttaactgtggggttggcgctgt |
| | ccttgtggtatcaaaggagcagacagagcagattctgagggntatccagcagcacaaggaagaagcctgggtgattggcagt |
| | gtggttgcacgagctgaaggttccccacgtgtgaaagtcaagaatctgattgaaagcatgcaaataaatgggtcagtgttga |
| | agaatggctccctgacaaatcatttc |
| 212654_at | ggtggccgagagtaaatgtggggacctagaggaggagctgaaaattgttaccaacaacttgaaatccctggaggcccaggcg |
| SEQ ID | gacaagtattccaccaaagaagataaatatgaagaggagatcaaactgttggaggagaagctgaaggaggctgagacccgag |
| NO: 112 | cagagtttgccgagaggtctgtggcaaagttggagaaaaccatcgatgacctagaagatgaagtctatgcccagaagatgaa |
| | gtacaaggccattagcgaggaactggacaacgcactcaatgacatcacctccctctgagccccacgccagcgtggccacctc |
| | agctctcttctctcctctcctttccattctctctatggggaggggagcaggcaggaggagcagaaattgccaacattgcaca |
| | gccaggctgggagcagcctagggagagcccc |
| 212671_s_at | accaatgaggttcctgaggtcacagtgttttccaagtctcccgtgacactgggtcagcccaacaccctcatctgtcttgtgg |
| SEQ ID | acaacatctttcctcctgtggtcaacatcacntggctgagcaatgggcactcagtcacagaaggtgtttctgagaccagctt |
| NO: 113 | cctctccaagagtgatcattccttcttcaagatcagttacctcaccttcctcccttctgntgatgagatttatgactgcaag |
| | gtggagcactggggcctggatgagcctcttctgaaacactgggagcctg |
| 212684_at | gactatttcaaagctactgttcctagtccagctttaagtttcggtaagaaacatgctgttttgtttcatgatttcgttaatt |
| SEQ ID | atggaaatttggcattgagggattattttattgagggtagaagagattccagaatcatcatctgtgatgatggtgtccttta |
| NO: 114 | gggctcttggagcagccagaccatgtttccaagagaaacctggtgatattgccagcagaccccctgccatccccccnagttg |
| | tcctggggctgaatgggcaaatctgtccaaacagctagtaaccggctgtgagggagagggtcagaagcacttagcgttggcc |
| | tctgattgctgtcctctcttgtcctcttcccactccaatgatgaaaatgattttctctaaatgcctgggtaaggatgctttc |
| | aaggagctcacttggcctgctt |
| 212775_at | tcaccgtcacagagtcttaccaaagtcaggacagttcaaataacaatccggagttatgcgtcctcttgaaaaagccgaagac |
| SEQ ID | ccggcggctctggtcccgcttccccccatggcgacgaacagctggcactgagtagcagctgc |
| NO: 115 | |
| 212776_s_at | tctcctcgtggatcgtgtatcccagcggcaaggtgtatgtggcagcngtgcgcctggagcgtgtggtgctgacctgtgagct |
| SEQ ID | atgccggccctgggcagaggtgcgctggaccaaggatggagaggaggtggtggagagccccgcgctgctcctgcagaaggaa |
| NO: 116 | gacactgtccgccgcctggtgctgcccgctgtccagctcgaggactccggcgagtacttgtgtgaaattgacgatgagtcgg |
| | cctcctt |
| 212801_at | caaggcatctgttgctttgggtcctccacgactcttaggcccgcctcaacaacccaggcacctcctaggtaggctcaaaggt |
| SEQ ID | agacccgtttccaccgcagcaggtgaacatgaccgtgttttcaactgtgtccacagttcagatccctttccagattgcaacc |
| NO: 117 | tggcctgcatcccagctccttcctgctcgtgtcttaacctaagtgctttcttgtttgaaacgcctacaaacctccatgtggt |
| | agctcctttggcaaatgtcctgctgtggcgttttatgtgttgcttggagtctgtggggtcgtactccctcccctcccgtccc |
| | cagggcagatttgattgaatgtttgctgaagttttgtctcttggtccacagtatttggaaaggtcactgaaaatgggtcttt |
| | cagtcttggcatttcatttaggatctccatgagaaatgggcttcttgagccctgaaaatgtatattgtgtgtctcatctgtg |
| | aa |
| 212877_at | gaggaaaatgaagctcgggctggttaactgacttgctcagcgtcccatggcctagccgcccgtgactctcacactgtctcct |
| SEQ ID | gcatgacgggtggcgcctcccgcagcttcccttctctctccagtgctgcccgctgtgtctagcagcctctaggatcttgtca |
| NO: 118 | gagctgcacctctctgtgaactggccattcctttcggtgctgctgtcctttttgggggggttcctgatttctgtatacatgt |
| | agctttgccagatatgtacttagtaatataaactgtattaataaaatccatttactgtgt |
| 213077_at | acaggtttgatggcacttctcatgatacattttagttattcttataaaagcaaacaggcaaacatgagtgtaaattaaagac |
| SEQ ID | aaaaagaaaactctggttttatatttgagancacgtgaaaaatcatgggtcaacataaaatcttgagaaccttctactttct |
| NO: 119 | cngggaaagcattatatagtggtgcattagtttagaaagtcagctatgattttgcctatagttctagttattagctttgggg |
| | ttttcttgtactttaagacatacctgtaaattgaacctatttgaattatattccactgtatgtgtattatggctcttttcct |
| | attagagcaacttgtgtttccctgataatgtgtacattttttaggcatgtacttaatagttcacaatgttctaaatttggaa |
| | ggacttaaaaaaaaaacttgtttaaatttccatctgttttgtaatatctagctctata |
| 213196_at | ctccccaagctcctttaaccaggagcttccctcccagagtgagctacgtcttgggaggattgaagcagggaggtggcagtgg |
| SEQ ID | ctagagttgctcagatcggctcagatgaaggcccaggggtcaaagcatttgctcaccacaactgagtctggaaggcactgtg |
| NO: 120 | cagccaaaccctcttgtccaatcacatgcaggccccaggcccctcagaaacgccctggtggagggaggagcccaacagatag |
| | ggtcagggagtcagtggggaaagcagaggggagagcttagagtgaaacttgaaactgctatggacatggaggtcagatggga |
| | acttggaactgggcatgaatcctgaatggtggggaaacttgaagccatcacagccaagaggtggggttccatgacctgccct |
| | gaggtcagcagctcccagactcctttgccctcactagggaagccccagtcaccggacttgtccccatggagtgaagagaggc |
| | cccatttttgagtgttgtgtgtccaaaacagtgttgtgtcactggtggtcatgttgc |
| 213295_at | tggagggattgtcctttcaagcaccacagcttcagataaaattagtactttcaaatattgtccactttaacttaaaaaattc |
| SEQ ID | tagagggattatattggagactcaactgcccttggttttagtttataaaatggcctagtactgtggaattttaattttagaa |
| NO: 121 | agtcttagcatcagatcataaacattcattaaaagaactcacatcccatctgaaacttcccaggggagttgggattcttagt |
| | agattggtagaaaggggctcattttctactgcatttcccatttttggtatcttgttcagcatgttttatttttatttcttgt |
| | ctgcagaacatcctatatttatgagaacattctttaagaagaccaccacatagaataccccttcctatcagctcgctctgat |
| | ttagccttaat |
| 213547_at | tggttccttcagagggtgtctctgcctcacannctagtagtatttagaaataggctgtgctgtcagctgtaaaagatcagga |
| SEQ ID | ggcagcagacaccactctggtttcttcactgcattcagcaatgcctgaagttagtgctcagnccgggcatctcaaaagaaaa |
| NO: 122 | gatacttgagttattcacattttaaaattcaaaacggttcatttttaagtggcagtgatgaatcagaaatttggaagatgat |
| | acgggtttcttttttccagggaggaggaatgggttgggtagggaactggacaggcttggacctcatgtttcatttctaattt |
| | caaaatacttattagcaaattgggcaacaatgggcatcttccatgccaccacccaggcataaccagttggtttgtttccttc |
| | tgaggaaggtttcaaatgtgtctagtgttcagtattgaggacaaagaaatacaagtggcaggcccaagtattttctgtgata |
| | tcccaggtta |
| 213551_x_at | gaggtctcagctcttggaggagggctaaggctttagcattgtgaagcgctgcacccccaccaaccttaccctcaccggggaa |
| SEQ ID | ccctcactagcaggactggtggtggagtctcacctggggcctagagtggaagtgggggtgggttaacctcacacaagcacag |
| NO: 123 | atcccagactttgccagaggnaaacannccttccaattgcccctccacccccagctgaggcccggtcacctggtcaggacag |
| | agcaactgcatctaaaagcacaagaagacagaaacctgtaagctctgaccccacccccaccccttgagaggtcagcggacca |
| | cctccttagggacagaccctggcaggtcgctgcccaccgagatttcctcaagtgtgcatagatctgagaggagtcgggagtc |
| | gagactcgagattccatcatagcgtaggtgtgtggggttgggagccccctgatgggcttgtctgtgtttgcaccttgtcctg |
| | tgtctgaggtcctgtgactgtaccctcctttgccctgggacatctgtatc |
| 213569_at | ctaggcttgctttatcatcacatcccaaggccagagggctgctgctgtccccatcttgngccctgctagaagagggatagng |
| SEQ ID | ggtggctggcatgatgnggtgaggggnnnannagaatatgcagaggccttggaggaagaggactggcagttatgacaggaag |
| NO: 124 | gctctctatacctggctccccagtgttctgcccctggcactgagcatgaggagccaggctttggggagactttgcaatcacc |
| | cccccaacctggtccattttccacaggtagctttcttgaactcaccttgacccctcctcagccagcagcccccacctccagg |
| | ggcaaaggagctgaaagacagtcctgaactggggggagctgggatcacatcagccaggccctgtccctcacaggaagtgaga |
| | tgaggtgataccatggatggtgactaaggccccaaagtccct |
| 213587_s_at | aggggaagaaacgacagcctcacttctgtatggactgctgatgtggcctgccatcctgttcagcgggcattgtctttggagc |
| SEQ ID | agcaggagantaggatgcctctcactcacatgccagttcctggctggccagctgctcagggctcaggctggggcctcccatt |
| NO: 125 | gacatcctccccctacactccctctntgagcctccgtcgcccctcctgttgggtaagggtgttgagtgtgacttgtgctgaa |
| | aacctggttcatatata |
| 213618_at | caatctatattcacaggcccatacttcagtcagtccaatcatagtacagtgatcgaccaanngnnnnnnnnnnnnncnnnnt |
| SEQ ID | tgtaaaatacggatcatttgtattttggggtgataaaatagttcaccatgggtatgagatatttattctttaaatcaaagta |
| NO: 126 | aattagaatttttaaaaagcacaaaactgcaggacagtttatgaaataggtggcactattagggaatcttcctttaaagcaa |
| | gaaatcatgttatttagaaagaaaaactaatcttaaacatactatttctaataaatatttatatttttatgaaataaagagg |
| | tatgtggaaattaatatttggtgatgttggacagtggaaaagtatctagagtttttacctgccttatctgaattcttcttga |
| | aacttgagcttaaactctaatagctgtttccctttctattctgaacaactgtctccatttttcaa |
| 214131_at | tttgatggggacagcccacgtctgatagggtgggacatggggcagcagttagagggttgtgctctttctagtgtgggatagt |
| SEQ ID | ttgcaagatgatatgttgtagccacttctgcagtgacggggacagaaggagtcagtgttcttaactacagtggtagcagcag |
| NO: 127 | aaaagaagggtaagcagggttctagaaatttgtgttatgttttctccccactgtatttatttctttggttagtggtgcaaga |
| | aattctgttttcctgtagcaaattaataaagcgttcaaacataaggaattacgacaacagcttgtagatgccagacttcaac |
| | aaacagcacagctgataaaagaagctgatgaaagacatcagagagagagagagtttgtaagttctacttct |
| 214218_s_at | gtgccagacttctgagaagcacctgccagcaacagcttccttctttgagcttagtccatccctcatgaaaaatgactgacca |
| SEQ ID | ctgctgggcagcaggagggatgatgaccaactaattcccaaaccccagtctcattggtaccagccttggggaaccacctaca |
| NO: 128 | cttgagccacaattggttttgaagtgcatttacaagtttctggcatcactaccactactgattaaacaagaataagagaaca |
| | ttttatcatcatctgctttattcacataaatgaagttgtgatgaataaatctgcttttatgcagacacaaggaattaagtgg |
| | cttcgtcattgtccttctacctcaaagataatttattccaaaagctaagataaatggaagactcttgaacttgtgaactgat |
| | gtgaaatgcagaatctcttttgagtctttgctgtttggaagattgaaaaatattgttcagcatgggtgaccaccagaaagta |
| | atcttaagccatctagatgtcacaattgaaac |
| 214901_at | cgaagccggcggcgtgaacaatcctcgagcaggaactcacacctggttcagcatcaacacccgaactccagaaagagctctg |
| SEQ ID | caggcggagcaaaggcagggcagccggaaagcagagccctggctttgtttgacatccaaaaaatcatgcaagagaaaaaccc |
| NO: 129 | tgtgcacgttattggggtggaagagccttctgtgggtgcttccatgttatttgacatcagagaatccacataggagagaaac |
| | tttgctgatgacttttaaccacaagtaaaaaatgtggtaagtccacatagtgtactcatggaaggaggggctgggggtagaa |
| | atgtcatgggtgacttctgactttctaaggaaatgatgcttcccaagcacccgaggttggttggtcccaaatctatcaaact |
| | cagtgccctctttagcgacatattttgtgacattccttccattacaccacagtgagttcacaggtaatataacctacccacc |
| | tgtgtaatgtcaaaaaaaatcaatatgcggccccattttgta |
| 214983_at | gttccttttcgattcttggtcttatttcttcacccatctactatagtgcattcttgccatgcagccctttttatggagcaag |
| SEQ ID | tcacaatacagcagcttgcttcattagggcaagcaagcaagacaagctgcagcaaatgcaagtaacatggaagtctttataa |
| NO: 130 | tctaatcatggaattgacatagttaaaaacaaatcattaggtaggctccaaattgatcagaacatggttattggacaaaacc |
| | atgactgtcaggaggctgcatcatggggagccattttacaagcagcaccatgggtgttatgggggattttattacatttgtt |
| | ctgctcttaagagttgaaagtctttaaaaatgtgtaagtctgtcgtttgttcttgacttctgtcatgttttcaagaatgcat |
| | tatgcaatgatgtagaatactgtttgtaaagtagttgtctagactctagtgaaaataattacagataatctcagttcatcaa |
| | cgaatcggt |
| 215314_at | ctcagcctcctaaggagctatttttngnattttagtagagncagggtttcnccgtgtnggncaggctggtctcgaactcctg |
| SEQ ID | ncctcaagtgatccacccgccttggcctcccaaagtgctgggattacaggtgtgagccaccgtgcccagctagattctttac |
| NO: 131 | tttttaataaagttcaaaatagcttttaataaggttgtcaaatttgaccaaaatttcatagaagtccaaaatattatgcctc |
| | tttctgtagatcaaatgaaatatggcccaatttacaagggtatacaccttcatggtagatgaatatagattttaaactagtt |
| | ttaaaaggaatatgaatgatttaattgtagtttctgctgccaaaaagtctccataattattttttaataacttataactacc |
| | ttgatctttacctgtacacacacacacacacacannnnnntgcacagagtaatttcacttagaatcctggtca |
| 215359_x_at | ggactcacactggagaaagaccctatgaatgtaaacactgtggtaaagccttnagtcgttccagtttctgtcgagaacantg |
| SEQ ID | aaagaactcacantgnagagaagccctatgaatgtaaggaatgtgggaaagccttcagttctctcagttcctttaatagaca |
| NO: 132 | taaaaggacacactggaaggatattctataagtgtatggaatgtgggaaagcattcattggttttatcacattcagatactt |
| | gaaagaaataaatcctgtgaatgtaaacgtggtaaagccttaagaagtttccaggctgggcgcagcggctcacacctgtaat |
| | cccagcactttgagaggccgaggagggcagatcacgaggccaggagatcgagaccagcctggctaacatgggaaaccctgtc |
| | tctactaaaaatacggaaaaaaaaaaaaantagccaggcatagttgctcacacctgtagt |
| 215666_at | gaaaaatccgtggaactgaagctgagatctttagtacgtggagtcaccttacagatacagagcatttatgcggtattcattg |
| SEQ ID | gtgcctaaagaactttaggcatcctctggaaaaccggcc |
| NO: 133 | |
| 216438_s_at | acagagacgcaagagaaaaatccactgccttccaaagaaacgattgaacaggagaagcaagcaggcgaatcgtaatgaggcg |
| SEQ ID | tgtgccgtcaatatgcactgtacattccacaagcattgccttcttattttacttcttttagctgtttaactctgtaagatgc |
| NO: 134 | aaagaggttggatcaagtttaaatgactgtgctgcccctttcacatcaaagaactactgacaacgaaggccgcgactgcctc |
| | tcccatctgtctatctggctggcagggaaggaaagaacttgcatgttggtgaaggaagaagtggggtgggacaacagtgaaa |
| | tctaaagtaaaaccaagctggcccaaggtgtcctgcaggctgtaatgcagtttaatcagagtg |
| 216540_at | gagaagaagtccccaacatgactgtaacttgttgattctttatatttcaagaggaagtgtccaaaactgatctcattcctct |
| SEQ ID | cctttcatcctgggagcagtgggtgtccagagcaaacaaaaagaacttattttggctggacagcaagcagagtgccagttcc |
| NO: 135 | aaagatgagcttgtttgtagcaccaccataaatttgggaagcacagtaatacatggctgagtcttcctcttgagcaaatggt |
| | ttcttcaggtggaaagatgtctcgcctttgttaaggtcagcag |
| 216958_s_at | tgtaatcccagttacacagaagactgaggcaggagaatcgcttgaacgcaggaggcagaggttgcaggagctgagatcgcgc |
| SEQ ID | cattgcactccagcctgggcaacagagtgagactctgtctcaaaaaataataataaaataaatgaacacacatgctgctgag |
| NO: 136 | tccgcaggggggcagagcagaggacagcgtgcttttgtgtactgttggaagactggctcctcctgtacagcacctctgagcc |
| | cttgtgcaccgccctgccacgggcaccatccagtcctggccgtgtgaccacccacagctgactgggcagcaggcacaggccc |
| | tacccgagcaggccggagttggctcgcatgactccagctgaggctgcctgtgtacatttctccagataccctatggctaatt |
| | ttgttataactgcacag |
| 217147_s_at | tctcctttctcaccaatgggcaatagcccataattgaaataaatttctgattgaaaggtataggaaacattaaaatgcatta |
| SEQ ID | ctaagagaagtaatataattttcttacaaagtatttttcccaaagatagctttactatttcaaaaattgtcaaattaatgca |
| NO: 137 | tgctccttacaacaaacaaatatcaaaaagagtttaggaattctactagccagagatagtcacttggagaaactttctatat |
| | atccttctaaatatttttctgggcatgctcatgtatgtacatcagttgtttctttttattttgaaccaaaaatgtggtttct |
| | tttgtacacattacttaaactttctttccagtcaacaatatattgtggatttattttcactgttatatttaactatatataa |
| | atacgcatatattgtaattttaatgtctgcttagcaccccactgataaccaaatcacag |
| 217778_at | tagggactggcgccaaatggtctctccctgccaattttggtatcttctctggcctctccagtcctgcttactcctctatttt |
| SEQ ID | taaagtgccaaacaaatccccttcctctttctcaaagcacagtaatgtggcactgagccctacccagcacctcagtgaaggg |
| NO: 138 | ggcctgcttgcctttttttttgttncccggatcctggggtggggcagaaatattttctgggctggggtaggaggaaggttgt |
| | tagcatctactgctgccgtaccctaggaatatggggacatggacatggtgtcccatgcccagatgataaacactgagctgcc |
| | aaaacatttttt |
| 218553_s_at | agtcctgtctgtgtggactggcacctgggctgctggagaagtctcctcccgttcggaccagcctcagggctgcacgttacct |
| SEQ ID | caggaatgggccccaccatgaaggggcccatctgtcagcagcgtcttctaggtccccagctcagggagccatccccagctcc |
| NO: 139 | agttttctcatgcgaatatgcacagttttaattcacgttgttacactagcctgccgatgagacccagacacaggcagacctg |
| | gcgctcttgacccctgattccagtgaggactggccctgaggagtccttgcagacctgctgcctgccccacgacaggcccaaa |
| | gatggaccccccctggccttgtgacagctccccaagtgttctccggtggagaaactgcagaggactggtgggcggggctccc |
| | cagccatcaccatcctgtgtacaatggctgtagacttgtatatggctcctttaatattgtaaagatgctgatgtacaattgt |
| | cgtgcatttgtgtgaacacattgcgttcccagtc |
| 218723_s_at | actgaattctccaacagactctaccccagctcttctctctgccactgtcactcctcagaaagctaaattaggagacacaaaa |
| SEQ ID | gagctagaagccttcattgctgatcttgacaaaactttagcaagtatgtgaaacaagaagttctgggtcctttcatcataag |
| NO: 140 | ggagaagcttcagaaagttccgaggacctgctaaaatcagctactagaatctgctgccagaggggacaaagacgtgcactca |
| | accttctaccaggccactctcaggctcaccttaaaatcagcccttgatcccatttctgggcaatttagacagtgaaactgac |
| | tttgtttacctgcttgcagcatattagaacagacgatccatgctaatattgtattttctcttaaaacatagctttcctgtaa |
| | tttaaagtgcttttatgaaaatatttgtaattaattatatatagttggaaatagcagtaagctttcccattataat |
| 218786_at | tgctggctggccatttacttccagcccttatgaggagtttcccctgctgaagagccctgcctgccccagatcataccccctt |
| SEQ ID | cctgcctgtaacccttaccggctccatatggggtacaaaggtctggcctcctcaccccaacttgggaaaccctctggggcca |
| NO: 141 | tcccagctccagagccccttgtggggtcagtgagacctcattgtggccacattacagcccagtgcctctccctgacaagcct |
| | gtacccagccggctcagcccacagcactgtcctatgaaccttcctgcacgccattctccacctcagtatctgctttcgggga |
| | acccaacctgcgacagtgcttctgtgtgttttcagtcctgcaggtttgaactctgactttggagacttttccagttatctcg |
| | tggaat |
| 218838_s_at | gggcatggtaattagcctttccccatgttaatttattcagttttttcaagggtcaactgaattccccacttcctgggtaaga |
| SEQ ID | agcatgatctccttttaatttcacgtctaagatcctggcagcttcccctagctggttcctctgtagtcctgctgggactgtc |
| NO: 142 | agctcatttaaatgtgggtctgcagaaggctttaggtctcccccaacccccttacctttcacagaggaacctttcatcagga |
| | taaatgattattgctgccctgtgggtcttgctcaatactgttcatacctggagagagaaggtattgaaacatctcctttatg |
| | tgtgactttcccaaatttttaaaaattgtttatggtttaggccccttaaatactgtgtagcaggatgaagtctaccattacc |
| | agctgggtcaccttggatgggtctgtcaacatctaagcctcagttccctcacctgtaaaaatgagggtagtccctacctcat |
| | aaggg |
| 218956_s_at | ttccataggccgatgctctgaaagaagagacgtggggctcgagagatttaaagattttatttttacaaatcacagctgatag |
| SEQ ID | acagcgaagccttccccatagagaccgtgctccaactcgggcctggggcactgctcgctgctcccaggaagggggtggcgtg |
| NO: 143 | acaggcaggaacctgcgaagtccagagtccagggtggagcgcaccagcctcagccagagcagccacgacagccacagtgtgt |
| | gcactcgatgatgcggccctgcaacggaggaggacagtgagacgatgccactgcgccacgctcgcccctgcacactcacata |
| | tgtggcaaccctcccacgaaggacctgccaccatgccatatagggacacacctcagaaacccttccttgacagctctggaca |
| | gggaaaatttggctccctcatgaaggtagaaccagctgctgttgacaccgaggttacatctgtatgtctatttataatatgt |
| | tctgcaaatccaacacacgtt |
| 219343_at | gacagttcagaatcatgttccccattctggtgttggatctataggtttattagaatccttaccacagagtccagattatctt |
| SEQ ID | cagtattctatcagtacagctctctgcagcttaaactcggtggtacataaagaagatgatgaacccaaaatgatggacactg |
| NO: 144 | tataatttggttaagactgctgaggccaagtgctattttgttacaagaaaggaagaacttggctattttcttgacactttta |
| | tgggtgctgcactttatttttgtttggtttttgatgggagggaaagagtactgaaatgttttgtaaattttttttaatgtgc |
| | tgctaggttttttgttttgttttgttctgaagagaagagtggtaccatatgttgcaggaagtcaaactggactttttgtggc |
| | tactaaatttgcctttaatcttattgttctcaattttggaatcaagtatgaaaatctgcacaaatgcaatgtttacaagaac |
| | tggttgattctgggaggcatctgctacagtctctttttatatggatatgtacatgtcctattctac |
| 219368_at | agtgttgtgtagcttaatccttctgaagtctttttgtcatgtagctattaatctgtggctatgaaatgatcagaaatgctaa |
| SEQ ID | gtgagatcaatatttgtttggaaaaaaaatcttgggaaacaacccaagggttttcgctgttgttgtttttctttttctattt |
| NO: 145 | ttgtttacttagtcctttagctagtggatttaattttgttgtgcctgcttcattttgcaataacaatgcagtagaatttaaa |
| | acttggatgcttaagaggcctgcatatagataagaatttcaggcaaaactacatttattgttaataacagcttgttcatagg |
| | ctcttgtattttatgtaactgtgataaataatgaaacttagttatattgaggttattgtttgtcggtgaagtgttagtcaca |
| | gtattttcaaaagtttgcacatattgttctgtgtaattgtgtaagccataattacagtgtttaattctcttttcctattaca |
| | tcattcattgaaagtgatcactttaccattttgaaa |
| 219551_at | gaacaggtgaccataactctgccaaatatagaaagttgaaggaagtagtaaaattcagtatcgtaaagaacaacagcaacaa |
| SEQ ID | caaatgtggaattcagccaggactcccaatcttgtaaaacattctccatctgaagataagatgtccccagcatctccaatag |
| NO: 146 | atgatatcgaaagagaactgaaggcagaagctagtctaatggaccagatgagtagttgtgatagttcatcagattccaaaag |
| | ttcatcatcttcaagtagtgaggatagttctagtgactcagaagatgaagattgcaaatcctctacttctgatacagggaat |
| | tgtgtctcaggacatcctaccatgacacagtacaggattcctgatatagatgccagtcataatagatttcgagacaacagtg |
| | gccttctgatgaatacttt |
| 219844_at | atcaggcacactttcttcagaggcatctgattttaacaaagttcatttaagtagacggggtggcatcatggcatctttatat |
| SEQ ID | acatcccatccagctgacaatggattaacattggagctctctttggaaatcaaccgaaaattacaggctgttttggaggata |
| NO: 147 | cgttactaaaaaatattactttgaaggaaaatctacaaacacttggaacagaaatagaacgtcttattaaacaccagcatga |
| | actagaacagaggacaaagaaaacctaaaacaagcctcttgctcagtaaagagacaaaagccacacaggagtaggtgccact |
| | gacctctattgttggagactttgttccactttttgtttcagccagtaaaaatattgttttgcttcatctgtacacaaaaaaa |
| | tacccttttacaatatgaatgcattgctgtatatactgtaagactgaaagctttgatgaaatttgtttttgtatggtgcaat |
| | atgacagcctgtcattgaatctaaaca |
| 219901_at | agcagaggatgctcattcggctcagaagtggatagaagcatttcaggaaggcacaatattgtagcagtattggtttcatctc |
| SEQ ID | ttctgtgattccaaagaggtggaatttcatcagaatggagtaaatgcaattcaaaaattgtataaaaatgaacactgccaag |
| NO: 148 | ataaagccaaccagacccttcatcaaagaaattgttttgttaggtataagcaatttttaaaaggtgtttgttttttcattta |
| | tgttatttattaaaattttgatgtttacttaatggtcagaattatttctgagacacactgaattctaaagtaccatttcttt |
| | agagaccagaaaaactatcttaatactgtatactgtattaactattcgtgacatagttcacactgttttcttaccttacatt |
| | gtaacaatcttactggtggaaagtctttgtaaggaaaaaacacatagcaaggagcaaatttccacaaagtgcttggtttagg |
| | a |
| 220005_at | ttcttgcatccttctgtgattcaaaaaagtaaaatgtggctttctgaaatgatggataagagtctacatcttctagaaaaaa |
| SEQ ID | tacataaaggagtagttaagctctgtaaatgtgccacgagctccaacacgaccatcgtagggtgaagcccacgttttcttcc |
| NO: 149 | atggcctcaaaggccctagaacttgcctacctttctggccttacctcctagctacttatccatctcttgaactttatactct |
| | tgtataaatttctaactttcagaaaatgccatactctgttttggcaccacacatgtatatttccccctggtacacttggaag |
| | actcttatccatctgtgaaaccctatgttgtcatcacttggtccatgaaatattacctggccaatatcccaccatcacctca |
| | aacccaatcaccccctcctctgtatgctgtcacacctatattattaaacttatcacattgcattgtaattacttcctgacct |
| | ttgtatctactcttttagt |
| 220168_at | aatatctttcctactagacactctcatttttatgttattataaacaataaggttcctttggtagaagtgaaagcttatcgac |
| SEQ ID | agatggccctactaagttctgcttttgcatttggttggagcaagtggaacctactatgtaattctacaaaagtcgtatttaa |
| NO: 150 | ggtgagggaacaccttactgaagaatgtactgagaatcctaattgggcccttttaatgtttagtggtgacagagcacaaaga |
| | ctgaagatcaaggaagagagtgaggcattttctgaagcacttaaagaagaaactgagtttcattctactttatatcacatgg |
| | tgaaggattttgcttctgaggaagcaatggagaaagtcaggagttccaactgtcagtttgtcaactctgtgtgccacatgct |
| | gctctctaccagattgctcagctactcctaacctccacttacaaactgtattgtaagtatgaagaaccaagcatggtgaaag |
| | acaggtatttctgcaacacaatgta |
| 220241_at | cgccagtacatcttctagcattgtcgtggaagttctccgaatcctggttttgattggtcagattcttttttcactagcggcg |
| SEQ ID | gtttttcttttatgtcttgttataaagaagtatctcattggaccctattatcggaagctgcacatggaaagcaaggggaaca |
| NO: 151 | aagaaatcctgatcttgggaatatctgcctttatcttcttaatgttaacggtaattctcaaactatgtgttatttatgtaat |
| | ctgatacataatctctttcactgaaatcttgtgatccattctttacctttccattttaataatg |
| 220301_at | ataagtcatttctaatctttgtataaaacagaagtgagcaagatgaatcagaaaaaaggtgttttgtattttaaaggtaaca |
| SEQ ID | gataaccaggtgattgaatctaagacaggctgtaagcatcgctgagaaactaaaaggacttttgacttttatctggatagac |
| NO: 152 | atttctacagtaaaatcatggaaaggcatcagcattgcaaagtagcatctaggtagaaatcaggccaaaattaagctgtggt |
| | ttccctctgagtagtgggaatagagaaaattaggaaattgtggttatgtgaatatttctttaaaacttttatgtacattata |
| | gtttattgcttcatatttaagtttagtttttaaggtaaaatgttattttgaacaaaaagacacttataattttccataccta |
| | ttttcaactgaaggcaacttgtaagatttaactcagtcaataacatactggttttactcatc |
| 220933_s_at | gttgtttactgaatcctggtgtgaaagcatatcagatatgtatgaactgctactgctgtacttccgatttacggacatcatt |
| SEQ ID | ttattgctatttgtagacgtgataacatgaacatgagtacctatttatgtgggccttcagtggatgggcagtgccactcagg |
| NO: 153 | tctctggggtttccctctctaattttaagtaaattgacatataactactatgcttataaaaatgaagtaaggaaaacaagta |
| | gtcctgtttgccactaaaaacattttcaaaggaaaaataaaatgaaagtactttttactttttatgatactcagaaattagg |
| | atgaagaacttttaaaattgctgaaggtcaaagaggttatctctgccagtcacaagtgtggctggtgtcattctgggtctga |
| | ctggagccctcctggactgtttctttaatttcaaaagccctgcagacatagtacctggtcagaactatgcctcggt |
| 221205_at | aaagcttatccaccacgattaagccggcttcatccctgggatgcaaggctggttcagcatacacaaatgaataaacataatc |
| SEQ ID | catcacacaaacagaaccaatgacaaaagccacatgatatttacctgtatacctttttaagtacaaataaatctgggctgtc |
| NO: 154 | attatttatgctaacactggttttgtgtccctggaatctatctatctaagtttttttttctttttttctttttccattttcc |
| | agtacctattagacagaatggctttcaattttttctcttacttccaaacttagtca |
| 221728_x_at | attttctagttttcattctgtacatttttagttagacatcagatttgaaatattaatgtttacctttcaatgtgtggtatca |
| SEQ ID | gctggactcagtaacaccccntttcttcagctggggatggggaatggattattggaaaatggaaagaagaaagtaactaaaa |
| NO: 155 | gccttcctttcacagtttctggcatcactaccactactgattaaacaagaataagagaacattttatcatcatctgctttat |
| | tcacataaatgaagttgtgatgaataaatctgcttttatgcagacacaaggaattaagtggcttcgtcattgtccttctacc |
| | tcaaagataatttattccaaaagctaagataaatggaagactcttgaacttgtgaactgatgtgaaatgcagaatctctttt |
| | gagtctttgctgtttggaagattgaaaaatattgttcagcatgggtgaccaccaga |
| 221759_at | cagcctagccttcaagtggtgtgagcggcctgagtggatacacgtggatagccggccctttgcctccctgagccgtgactca |
| SEQ ID | ggggctgccctgggcctgggcattgccttgcactctccctgctatgcccaggtgcgtcgggcacagctgggaaatggccaga |
| NO: 156 | agatagcctgccttgtgctggccatggggctgctgggccccctggactggctgggccacccccctcagatcagcctcttcta |
| | cattttcaatttcctcaagtacaccctctggccatgcctagtcctggccctcgtgccctgggcagtgcacatgttcagtgcc |
| | caggaagcaccgcccatccactcttcctgacttcttgtgtgcctccctttcctttccctcccacaaagccaacactctgtga |
| | ccaccacactccaggaggcagccccatccccttccagcccctaagtaggccctcccctccctaaatctgcttccgcaccacc |
| | tggtcttagccccaaagatgggccttctctctc |
| 221789_x_at | ttctcactctacagggtcctggtgaagagccagtgaggcccctggtacccaagccccctcccctgacctgggtgtgnctcgc |
| SEQ ID | tgctggggctctgcaggggcagcacagctggggtgcaggccaggctgccactccgggaacgcctttgcgccgggactttttg |
| NO: 157 | tttctgaaggcagtcgatctgcagcggggccttatgctgccatgcactgccctggctcctgccggacccccagggtgggccg |
| | tggcaggtggctgagcaggagctcccaagtgccggccaccgctgtcagggattgcccacccctgggcatcatgtgtgtgggg |
| | ccggggagcacaggtgtgggagctggtgaccccagacccagaattctcagggctctacccccctttcctggtcctaggtggc |
| | cagtgggtatgaggagggctggaaggcagagctttgggccaaaagcaggcgttgggggntcccccctcaagtttggagccgt |
| | ttccgtggttgtagcagaggaccggaggttgggttcctgat |
| 221874_at | ctggcctacgtgttcaattttctatgaacaaaggctttagtccttgacccagggctaaagtggtctgtccaagctgttgttg |
| SEQ ID | gtagagggagtatgataaaatgtttaaatctcatttggttaccttgagtcctggaacacgcagtaactgtcatgctatagtc |
| NO: 158 | ntcatctgtatttggctgggaatacaaatgaagattgtggtgtattcaagcagtagggtttttgcttttgtttttgttttag |
| | tgccaacaaaacttttttttgtctgactacattaaagataagactgactatatttatacaacagaaactttgtaatagattt |
| | tttcagctttgtgaaatcgaattttttttcatcagggctggttggatttcctttttaccctgtaatccaagcgttaatagtt |
| | tgttagaagatgggttattgcatgtcactt |
| 221905_at | tctgggtttagggctagccctgcctccatctcccttgggtaaaatgaagggtgtggggtaaaagatgcataaggccnttttc |
| SEQ ID | tagctctgacagcctagaagtccnaatcaccctgtaataaatatgtgttgaatgaagaaatgggtgaatgagcttgtcaatg |
| NO: 159 | tgattttaaaaaattgactaccntggaggaatgattaggaatctaaatgaagccagccctcggtatctgcaggtttctcatc |
| | catggattcaaccaactgcaaatggaaaatacgattttttttaaaaaaaggatggttacatccgtattgaacatgtacagac |
| | ttttttcttgtcattattctctgaacaatacaagaactctttatgtagcatttacatttattaggtattntaagtaatctag |
| | agattatttaattaaaatatacaggaggatgtgtgtcagttatatgcaaattctgtaccattttgtatcagggaattgagca |
| | tcttcagatgttggtatct |
| 222462_s_at | tcagagtggtttcattgccttcctaccctctctaatggcccctccatttatttgactaaagcatcacacagtggcactagca |
| SEQ ID | ttataccaagagtatgagaaatacagtgctttatggctctaacattactgccttcagtatcaaggctgcctggagaaaggat |
| NO: 160 | ggcagcctcagggcttccttatgtcctccaccacaagagctccttgatgaaggtcatctttttcccctatcctgttcttccc |
| | ctccccgctcctaatggtacgtgggtacccaggctggttcttgggctaggtagtggggaccaagttcattacctccctatca |
| | gttctagcatagtaaactacggtaccagtgttagtgggaagagctgggttttcctagtatacccactgcatcctactcctac |
| | ctggtcaacccgctgcttccaggtatgggacctgctaagtgtggaattacctgataagggagagggaaatacaaggagggcc |
| | tctggtgttcctggcctcagccagctgcccacaagccataaaccaata |
| 222520_s_at | aacatgctcacatacacagatctttccctttccttactagacctctctttagtttaagataagtttagacaggagaaagtgt |
| SEQ ID | atgtaaactacctgaaaggatgacaatcaatacaatgacaaatatgccagtttatttaatttgtttaaaaaaaaaaaggcag |
| NO: 161 | aaaacccaccatgggaaaataagcattttttaaaatctgaagttgctcttttcctgtgtggtctttgggaggagtctagtac |
| | aaaaactgaaaacctttggtcctgggaatttattttcatggctgttgtcatggctgagtagttttttgatgaattcaatcaa |
| | tgcagagaacgcttttagataaatcagaactctccttaaaatgcatttcaatcagaagcaaagtacacccattatttatgca |
| | gacatgtaaaaaatctaaaaattcttgcaatgagtagtataagccacctatttttacttccttctctgaaatattccagtta |
| | actcagcttgggttctgtgctcctcctctcttcctatagaacactgtgcataccttgaataa |
| 222654_at | gagacacatgatgatttgcattgtaataatttctgtgtgtatgtgtgtgtgttgnnttgtttttataaagaaaagtgtgttt |
| SEQ ID | gtacccatgagttcagcatttctgccatcttgctattttcattttccctgtggaagatgcatggttgcatccttttccttct |
| NO: 162 | gaccaacacttagtctttaactttgtaaggttcggtttaaggtttcagggttagcactatagttttggttcattttctcatt |
| | aacgataaaatatgctgtaaggcactataagaatataaatgattctattacaattgaaagttacttgacattacatgaattg |
| | aactgcctttcattatcttcagacactttgttttagtatttttttttaatttgctaggtttgtatatagaagatcctttcat |
| | tatcaagaatgagtatggttgcttttgccatggttgcttttactattaataa |
| 222778_s_at | ttgtgaaccatttaacccttttgatccaaggccccggaccaaggggatactgtaatcaggttatgtacaccaacctctgcat |
| SEQ ID | cagttgcttctatagagcatcagccctcatgtttcattgtcttaatgtcggggagcagcctggtgtcctgccccaggtgtcc |
| NO: 163 | gcatttttgcatggtaaggaaaaggcttacttgcccttcagctcataaaagcagaaggtgtgctattcacagtaccatgcga |
| | gtagcccacagttaattcccggcagattctttggctgtgttttatctttaatgaacgtttctgactaatattttgttgtaaa |
| | ccattaagtaatgtaacgcatgtaagatgcttagggtgcctggccaggactccgtggtttcctgttgtgacagt |
| 222802_at | atgagtctacctcacctatattgcactctggcagaagtatttcccacatttaattattgcctccccaaactcttcccacccc |
| SEQ ID | tgctgcccctncctccatcccccatactaaatcctagcctcgtagaagtctggtctaatgtgtcagcagtagatataatatt |
| NO: 164 | ttcatggtaatctactagctctgatccataagaaaaaaaagatcattaaatcaggagattccctgtccttgatttttggaga |
| | cacaatggtatagggttgtttatgaaatatattgaaaagtaagtgtttgttacgctttaaagcagtaaaattattttccttt |
| | atataaccggctaatgaaagaggttggattgaattttgatgtacttatttttttatagatatttatattcaaacaatttatt |
| | ccttatatttaccatgttaaatatctgtttgggcaggccatattggtctatgtatttttaaaatatgtatttctaaatgaaa |
| | ttgagaacatgctttgttttgcctgtcaagg |
| 222858_s_at | cgatctcggtcgttcatctttaaatagatctttcttgccaaggaatgctctggcccaggagcaaggtggaatgtttccctga |
| SEQ ID | cgctgtgatctgcagcaggcttcaaatgaaaaccgactaaggattttctttcaaaaacaaatcagaagcagatgctgattgg |
| NO: 165 | gacccatataccacgttgctgactcacgttgctgcccttccatgatgttgccatctccttgagaacactgaagcaatcacca |
| | ttctgatagaaagtgcttaaaccaccactcttaggtctgctcactcttagaacacacaatggaagaggaagggtttttgttt |
| | tcactcattgtggtccccaagcctattgacactagttgcctagagtcccactgtgagtcatggtcagcctgtctgacatcca |
| | ggttgtgctattaaccaagaaggaaacagatacttggaggcttagatgacttctgcagga |
| 222859_s_at | ttatctttatccaacatttctccaaatggactgaaagaaacttttcaaggacagtgtattataacaatccctttcccagaat |
| SEQ ID | tagttgtatagggttggcccaagagatgtaagaaaaatctcgcattgctccctaagcacccnggnccttattaaagagcaac |
| NO: 166 | ttctatttccagtcgggggagtaacactaaagctacaagaaatatgtaataatgataggtaataatgtgttccaaagctttt |
| | tcaaactagaataaggaggcaaatagaagaatgagatactgatgtccacagttcattggcagaatctaaccccttctgttat |
| | cttttttaatactatttttgtttagatagaagtttcaaagaagataaaaatgcttgaagagcctgagagtaaaaagattatg |
| | ctgcaaagctatgatataaactgctcttgcagtccaaagggatacctgattaaagaagtttcttatttaaacatctcagacg |
| | caaaaattacat |
| 223095_at | tgcctgtagcattccagagctcactgcccttctagatgtgccttcccgcttggcttccagcggcttgtgctcactctgtctg |
| SEQ ID | ccaggtatgagaagaacacgtaagaccgccaccacactcaccctccctcaaggccctgtgccataggggtggccacccgacc |
| NO: 167 | tgcccccagaacttttggatactggaggcagttgcataggtctccctctctgggcaccaggactcagtccagcccaagacta |
| | ctctgggcagctcccatcccagtctggggccatttgcagactcaggaaaggatttctacagtgttctataaaagccaaaaga |
| | gagagtgggtttgggaagagtgagggtggttggggagaggggaccgatgtgcctcattgtttagtggtgattacaaatatgc |
| | ttttctggataaagtttggttgtttgc |
| 223198_x_at | gaagcctgacaccttcagggaccagctccaggagctctgcatcccccaagacctggtcggggacttggccagcgtggtattt |
| SEQ ID | gggagccagcggcccctccttgattctgtggcccagcagcagggggcctggctgccgcatgttgctgactttcggtggcggg |
| NO: 168 | tggatgtagcaatctccaccagtgccctggctcgctccctgcagccgagcgtcctgatgcagctgaagctttcagatgggtc |
| | agcataccgctttgaggtccccacagccaagttccaggagctgcggtacagcgtggccctggtcctaaaggagatggcagat |
| | ctggagaagaggtgtgagcgcagactgcaggactgacccctcacttgaccagtcccattcagatccggcttggacaggcacc |
| | tgagatggtgccaaagtgcagctgactcttcccacgacagccctgcccttcccatgaggcaggctcttcagtgagtgtttga |
| | acgtaattatgtagttttctgtttaattgaaaaagagagctatgcctttttttctttttgg |
| 223284_at | agaggcctgccgaggggaggagcctggcctctgtccacccgtcagcagtgtgaagtctgttgtgtttgagcttctcagagtg |
| SEQ ID | gaatgactccttttccttcctggccctcgggggcctctcgaggtcagcctctcccaaccccctacctcagctcctgtctgca |
| NO: 169 | ctgagaaacctccccgggtgatgtctgcaaagtctgtgctgtccgtgccccaggctgggagagctatctggggagggggaga |
| | ggaggccgagcagaatacaccccagagttagggtttgcgactccgcctccctgggacctggattgggtcagatgcctgtcct |
| | tggaggggacaaggttgactgcttaggaggcgcgacgcacagggctgccaggcctggcccctctctgggaaggttgagagct |
| | gagacgg |
| 223407_at | tgttggagcgcagggacctgtggcggcgggaggccgaggcccgcaagcagagccagccggaccctgccatgcccccaggcca |
| SEQ ID | cacgcgcatgcctgagaaccagcggctggaaacactgaccaagctgctccagagccagagccagctgctgcgtgagctggta |
| NO: 170 | ctgctgcctgctggggcagactcactgagagcccagagccaccgtgctgagctggaccggaagctggtgcaggtagaggagg |
| | ccatcaagatcttttctcggcccaaagtcttcgtgaagatggacgactgagcccctttgggggacagtggcaaggagctcca |
| | tgctgaggatcgccacatggctgcaaaggacagggttggacccaatcccagagcagaggtct |
| 223571_at | caaagtcatctgaacttccgtttccccagggcctccagctgccctcagacactgatgtctgtccccaggtgctctctgcccc |
| SEQ ID | tcatgcccctctcaccggcccagtgccccgactctccaggctttatcaaggtgctaaggcccgggtgggcagctcctcgtct |
| NO: 171 | cagagccctcctccggcctggtgctgcctttacaaacacctgcaggagaagggccacggaagccccaggctttagagccctc |
| | agcaggtctggggagctagagcaaaggagggacctcaggccttccgtttcttcttccagggtggggtggcctggtgttcccc |
| | tagccttccaaacccaggtggcctgcccttctccccagagggaggcggcctccgcccattggtgctcatgcagactctgggg |
| | ctgaggtgccccggggggtgatctctggtgctcacagccgagggagccgtggctccatggccagatgacggaaacagggtct |
| | gaccaagtgccaggaagacctgtgctataaaccaccctg |
| 223645_s_at | tttagcagtgcctgtgacgcattcctgtgctgacctggattcttccaatatgctgaacacttcctctaaaagagccccagga |
| SEQ ID | gtccatctggaggctgaccccaaaggaatgaatgaagtaaaatgctactcaaaagccctctccacaggatctcctctaggca |
| NO: 172 | ttgattaagattaagtgtgatcattgtactgatggatatattttgtgtacatgtttctcttttagttgtaactattgatttt |
| | gtaatgaaaatttcctctcctttttctaccatatctctgtttttttagaactactcaactgtgtggtaacagaaagcttctt |
| | accaatttccccaactatgttgcacatcagcctcattttccccctttattggaatgcatgttttcattgccttctcctttca |
| | aagtgtacgtttgtgtgttcatcaccttaaattatcttaatttgagacttttttataatggtttcgtaatgtgaaatcaaat |
| | actaatttaaactctggagcccataatatctacata |
| 223646_s_at | aaaatactggacccacttctttcagaagagatgaagataccttatatgccctaaagttaataccagcagtcatattttatca |
| SEQ ID | gatgtaaatctggatgtaagctcttaatgttatactaaggcagtttcttaggctgtgacacttctttgtggtacttgttttg |
| NO: 173 | tgtgaaaggtaaattttggggagaaaacaatgtgaaaaacagaacttgttctgttgtttttggcatactgtttatgttagat |
| | acactgtgttacaatacaatattacgaagatctgcattgtattttggaatttggtttcctttcagaattattgctctggcta |
| | gcattggaaacaacaacaacaacaaaacccaaaggaaccctttgcagaagattcccttgtaaatggccctgtggcatgccca |
| | gtatctgcaatgttctagaatagaagttggcaaacctctctgtttgccaagcctgcagagttgaacatgtccataaatgtat |
| | acaatctgaccctgttttttggccctgtttctggacactgtagctga |
| 224588_at | cggaactacatgccctaggatataaaaatgatgttatcattatagagtgctcacagaaggaaatgaagtaatataggtgtga |
| SEQ ID | gatccagaccaaaagtcatttaacaagtttattcagtgatgaaaacatgggacaaatggactaatataaggcagtgtactaa |
| NO: 174 | gctgagtagagagataaagtcctgtccagaagatacatgcttcctggcctgattgaggagatggaaaatttttgcaaaaaac |
| | aaggtgttgtggtcttccatccagtttcttaagtgctgatgataaaagtgaattagacccaccttgacctggcctacagaag |
| | taaaggagtaaaaataaatgcctcaggcgtgctttttgattcatttgataaacaaagcatcttttatgtggaatataccatt |
| | ctgggtcctgaggataagagagatgagggcattagatcactgacagctgaagatagaagaacatctttggtttgattgttta |
| | aataatatttcaatgcctattctctgcaaggtactatgtttcgt |
| 224590_at | ttacaaggtttgtctattttcagttctttactttttacatgctgacacatacatancactgcctaaatagatctctttcaga |
| SEQ ID | aacaatcctcagataacgcatagcaaaatggagatggagacatgatttctcatgcaacagcttctctaattataccttagaa |
| NO: 175 | atgttctcctttttatcatcaaatctgctcaagaagggctttttatagtagaataatatcagtggatgaaaacagcttaaca |
| | ttttaccatgcttaagttttaagaataaaataaaaattggaaataattggccaaaattgaaaggaaaaatttttttaaaatt |
| | tctctaaatgtaggcctggctgggcttt |
| 224709_s_at | tcaagaggtcatttgttccccatagcagcatatctcatttttaaattgaagcgaattaaataggattttactactcaacatt |
| SEQ ID | cattatactgttaatctttgctgaaatatatgctaacaaatgttaagcaagggaaactgaagacttagtcatgtggattgtt |
| NO: 176 | agcagtgatctgcattctgtaaaagaggtactttcccatgatgtaggcatgaagtggtgccagtaagcgtagagcggaaatg |
| | ttgactttagttaacattgggtttagcatttccagtgcagcattatcagtgggcctttaaaaatacttcgtaagtacattag |
| | ctttcactttgttgttaaattatagcagactcattatagagaacaagtttgccttgattttgtttaaaatgacttctgctaa |
| | gcacccagaagataaaattgacatatttttataatataagcatactttttttgtacattgtgttcattcttgaataaaatga |
| | gttctgtgttggcttgtagatacta |
| 224859_at | gcaccctgcggtttgcagggggctcctgcctggctccctgctccacacctcctctgtggctcaaggcttcctggatacctca |
| SEQ ID | cccccatcccacccataattcttacccagagcatggggttggggcggaaacctggagagagggacatagcccctcgccacgg |
| NO: 177 | ctagagaatctggtggtgtccaaaatgtctgtccaggtgtgggcaggtgggcaggcaccaaggccctctggacctttcatag |
| | cagcagaaaaggcagagcctggggcagggcagggccaggaatgctttggggacaccgaggggactgccccccnacccccacc |
| | atggtgctattctggggctggggcagtcttttcctggcttncctctggccagctcctggcctctggtagagtgagacttcag |
| | acgttctgatgccttccggatgtcatct |
| 224940_s_at | aataaaactgtgatctcgtctagagaaaatgtattcatattacaaactgctcttccatatttatgtaccatattataccttt |
| SEQ ID | ttattattgttataattattatgggtatttctaattaatatgatgttgaaacctgtttggcaccttctggaagctaccaaaa |
| NO: 178 | aaatgacactccattgaagtgcttaaaag |
| 224941_at | gtctacttaagacttctggtcatttccaacttatagaggaagggagtctctaaaatctcttcttcagaaggcacctcacttc |
| SEQ ID | tcagacttaaaattccacatcaagtgttccattaaaagaagataaggcattctgagtgcaaacaaatgggggcttcttaaac |
| NO: 179 | tacacaccagcagtcagtgaggaaaactttgaacaattattgagttgctttcttgggtctctataatcaataacctgtctgc |
| | agatatctatcta |
| 225150_s_at | gtgagagagaaaggtgctggcataggatctgcccagaagagaaaatgacccatgcgcagttgggctctggatacggcgctgt |
| SEQ ID | ctatagcaagttggccagtctggcctcctgttcctctgctggacctggggtaggctgcaggggtgggcagaagcccctctta |
| NO: 180 | aattgtggttgccatggtaccgaggg |
| 225230_at | gactgttgttggttatctggtgtggagtaagtgcacttagcatgctgacttgctcatcagttttgcacagtggcaattttgg |
| SEQ ID | gactgatttagaacagaaactccattggaaccccgaggacaaaggttatgtgcttcacatgatcactactgcagcagaatgg |
| NO: 181 | tctatgtcattttccttctttggttttttcctgacttacattcgtgattttcagaaaatttctttacgggtggaagccaatt |
| | tacatggattaaccctctatgacactgcaccttgccctattaacaatgaacgaacacggctactttccagagatatttgatg |
| | aaaggataaaatatttctgtaatgattatgattctcagggattggggaaaggttcacagaagttgcttattcttctctgaaa |
| | ttttcaaccacttaatcaaggctgacagtaacactgatgaatgctgataatcaggaaacatgaaagaagccatttgatagat |
| | tattctaaaggatatcatcaagaagactattaaaaacacctatgcctatacttttttat |
| 225475_at | aagacattccactattatgcgtaatgctcagctttttgtaaagaaatatttaattgtattttgtgtttatacaggtatttca |
| SEQ ID | cacaataatttgttcttaatgcagccactataacttgataagtcattgcactatttaaaaagttttagtaatatcatgaatt |
| NO: 182 | taatttgcttaagatttagtacatttcagaacttttgaacttttgacaaattgcattgggaaaagaagtctgttaatagtga |
| | tttagtctccccgcctcattcccacccccaacaaagcactatcattttcatttggaatgggtattttctgtttcatgatgta |
| | ttttcaaatagagttcaggatctgcaattcagttcaacacaagtttgttgagctttaaaatgtatttgaagtaatatttaaa |
| | taggcatttaaaatttcaaattagatgttaaatttatgcatagtaattttgcactgtaaaataattcccttctcccattcct |
| | tgtctgccattctgaatatg |
| 225531_at | cagagatctgtggggaagctccgcccagccacactccttgggataatactagccggttctgcctgattccttttccccngga |
| SEQ ID | gccagcctagggggcccgggactcctctagtgagccttgactgttaggtaagagacaggaagcagacaagccaagaggttgc |
| NO: 183 | tgcagctgcccccaggaggaaacgggcagcagggagtgtggcccagcccccnnactgtacccctccaggggcccgagccctt |
| | gccagcccaatgacaccttgaagtcaccacttttcctttcttgcccgctaataaaacctatttaaacaggaaattcatacca |
| | aatgcaaatgggttttgttttccttggcattttgttgttccgatcacagatttccctgccagggtgtctgtggttatcagct |
| | gcaggctcagcttggggcggccgcttcacacaagccactctgtaccacgtgccctaccttagtgacgggag |
| 225736_at | cctctggatattgatgcctcgggtgtggttggactgtcatttagtggacaccgaatccagagtgccactgtgctcctcaacg |
| SEQ ID | aggacgtcagtgatgagaagactgctgaggctgcgatgcagcgcctcaaagcggccaacattccagagcataacaccattgg |
| NO: 184 | cttcatgtttgcatgcgttggcaggggctttcagtattacagagccaaggggaatgttgaggctgatgcatttagaaagttt |
| | tttcctagtgttcccttattcggcttctttggaaatggagaaattggatgtgatcggatagtcactgggaactttatattga |
| | ggaaatgtaatgaggtaaaagatgatgatctgtttcatagctatacaacaataatggcactcatacatctggggtcat |
| 225882_at | gcaccttacatatttgatgctcaggaagaaagtgcaagaagatccctaatagaacattaaagattcttaaaagtttttgaag |
| SEQ ID | taggctgcttggggtgaccagccccacccttacatctccatagttggtacagttagcttgtagcagctgaagctgatgcctg |
| NO: 185 | gagatcactgtctgttggtctgatctcagtatcatactgagacacctcccctgagccttacctacttaaattggtaaccgtc |
| | ctaccaaaatttgtcaattttaatcaagtgaggcaagttgcaagggagccagagatacgtgaaaaaagaaaagcagaaatac |
| | tgatactttctaagaaagaagttgtaataatttctttggcacattgacttactgatatcttttgaaatgcatagangactct |
| | tgtgaaccaaagagagcagtagtggttattccccggcgcaataaaaatgcc |
| 226042_at | ttcccttcagtcttaaattgtctccagcgatgggaagaggccaggnactgtaacccttgtgctgtgtattctctgagcctct |
| SEQ ID | gctcactctcagggccaagcagctcccaagccggggccctctcttggccaaaatctgaggagcagtctaggttacaggcttt |
| NO: 186 | ttggtaggtaggttctggctgcctgttaatgcagttaggccccctgattaggtacagtgagaaacaagctagaacaaccctg |
| | gcccagaagactgtccactccagcaagatccagggatgatagccttgcagggccactgggagtttgtgcccaagcttctccc |
| | tcttctctccccagggggcactgggactggtccctgccctcatccttagcctgggccttccccagaggtattaaagagaagt |
| | atgattcctctgtcttcagttcttttcaggggcatcctgcccatagtacccagttcccaaggggcccccagtcacgtggtga |
| | agcctagcactcatgcagctct |
| 226043_at | agtgggggggtggtcagtggaagctggggaggcttcactcagctcaaccctgcagaccccccatatgagaacccccctcctc |
| SEQ ID | ccaccagctgggggagatgacatcactgctctctccatggggagggggctcacagggcaccaagttcgggatcctaggactg |
| NO: 187 | gcaggacctgctggagcctgagatgggccccaggggcctgaggttgcctcctcctgtccagacccagccttggcacctccca |
| | agactgccaagggccctaccatggccccacagcccccagggctggcagttccatctaggagggtgcctctaggccccactct |
| | caggctaggatggagaggcagatctgggcctggngaggtgacctgcttctggaggggcagggccgcacccgagagcagggac |
| | aggtgcccgaacacagggtctccaggacgtagcgcccccccgcatacttgaatgtatgtgcgtatttattgctcacgtctgt |
| | gccatgttgtcaatgggtcctttccaacccaagaggtacatttg |
| 226118_at | aggagacggacctgtgagtctgaccnngagncggncccnttcaccttggctgggcctggtcctggtccttaggttttgtcag |
| SEQ ID | gttgtccttgtttggatccctcaactaggtgataagcactggagggggatgacccgccttggacgtgtttctttaacctcat |
| NO: 188 | ccatataatagggccgtgggatggttgtagaggtaaagcaggatgatggtgttttaagaccagagcttgggaccagggctcc |
| | tacacctaattttctctcctggtagctgaacaaaggtctaaattagcttaacaaaagaacaggctgccgtcagccagagttc |
| | tgaaggccatgctttcagtttcccttgttgacaattgctctccagttcctatgaaagcacagagccttagggggcctggcca |
| | cagaacacaaccatcttaggcctgagctgtgaacagcagggggttgtgtgtctgttctgtttctctgcttgccgaactttct |
| | caataa |
| 226152_at | ggtagtttctgaacctattgacattgttcaaaatggatcatgtgccatattttgttagttgacatctgagttttcagtaaaa |
| SEQ ID | tgattatggaattaatcagcaaatgtagaagaatatattcaaagttaaaattcagtggcagcacagattatttttatcagag |
| NO: 189 | ctgtaaagaaaacaactgtccttttnctccccaccacccctcctgccccactttggcccagaaaccaaatgtgaacttcctg |
| | tctcccacctcagcactagtccatgccaggacaccagctgacaatttcttggttttactgtcaataattgtaccatgtgatc |
| | aattactgtcctcacttagaacaaagcctgagtccgagaatatttatattttaccaatatatgcctgttacaagagaaggaa |
| | atatgagttatttaagtttaacttttttatgtgaattcagagtttatttatcgagggaaatatgtacaaagaagcttcaaat |
| | ggaatatttaccgacattccttatacatgacagacacttggctacatgggaa |
| 226207_at | ccagccggagtcgggcatcaagcgactgtttagcttcttctcccgagataagaagcgcctggccaacacacagagaaacgtg |
| SEQ ID | cacatccaggagtcctttggacagtgggcaaacacccaccgcgatgacggttacacagagcaaggacaggaagccctgcagc |
| NO: 190 | atctgtgaccttggcccatctccaccctccaacctggactgcccgccaccagcgcctgcaaccgaactgcagcccaggggtc |
| | attgctgcctcaagcctctcggtgcagatgcaccctgaaaactgacccctcaaacagactgtctgatttgaggatggacatt |
| | gaaaaactgacgccaaactctaaagaaatgtttatttatacccagggctatcactgtttctaatagatgactctgatcccgt |
| | aggatatatatttaataatcccacaaacggaggccagacttctgcgttaacttcagtaacacaagcttctttaagccaaata |
| | catcacttgccacta |
| 226470_at | ctggccccgagcttagggatgtgcttgcaaacccttctcaagggtctcacaaccccaacatcttcagactggcctgacctgg |
| SEQ ID | gccttgtcttccagttcctttctcccattcccagcctcatttcttaaatgactaggaattttttaatggaccatcataggga |
| NO: 191 | gggggtgctcctcttttcccaccaggttgaggtgggggccttgcatcgggggtccccagggtatg |
| 226515_at | cttcctcgcagcaagcggctggagatagagaagagcttactggtgcgagcgtccgtcgaccccgtcgccgctgacctagaga |
| SEQ ID | tggcagccggtctcaccgacatatttcagcatgatacatactgtggtgatgtctggaacaccaacaaacgccagaatggcag |
| NO: 192 | actcatgtggctctatctcaaatactgggaactcgttgtcgaactgaagaagtttaagagagtagaggaagccatactagaa |
| | aagtaagacaagagtgaaatcaaactgcttttagtgactcgaggccaggcagtcatgcgccttctgggtctccggcgtcttc |
| | cgttcccgtgctgcccgtgtcatggccacaccgtcacccttcagcagcgacctccactcccgccaccgtctgagcagaagtg |
| | caccgaagcctcagagacagagggtctcctcccgatgctctgccgctgttggggatatggtttcttgaagcatttttaggct |
| | gccagtattgtattaagcagaacagtataacctcgtattttagctccag |
| 226538_at | ttaaccagcaagtttctgctttttaaggttacttttagaataaatcatcagggaaacagagaggatgctttgctttgggttg |
| SEQ ID | tagtcaaaaactgattaaataatttaatgtctctggcacacactaaaaaccatacacttcagttgtgatctcagtggcatat |
| NO: 193 | ttatttggttaggtttcgttacatttattattacagatgttcagttgaccaagtagttcagtgttttctttcctttttttgg |
| | aaattttagtttgagtttgtgactgcagtgttcaagaactcagcatccttgttttctacaaatactgattaaaataaaatgc |
| | tgtaaaatgtgatgtaaaacattatcatgatcttcccatgcctttgttgtacttgtgccgaagtgttttgatattcctttgt |
| | ctggaagaaaatgtttgctttcattttgatcattttgttcaccttggaatcaacagg |
| 226854_at | aaaggattttgtggagtcgccccaggcctgacggcgttaattatccctggctttcatcttgatgctcttgcaggggaggctc |
| SEQ ID | aagatgccttgtggctcgacccctgcacggagcccccagcccagcctccccgtgggttgaaaccttgatctgggcctgactg |
| NO: 194 | tggctgctctggggacatgccctcgtcctttgctgaagctgaggctccagagggggagtcactgccacgaaggttgcccaga |
| | actttccttgctgcaaaaagccccaagacttctccctgccacacacacagcttttgccatcgcgttctaaaagctgacagct |
| | ttctctccaacactgttctccctgagtagggt |
| 226971_at | ggaggcctgaatacaggcggtcacctaaggtggtgcgtacagtgttgggccacatgtctagtggtgccaggactgagagatc |
| SEQ ID | acttacctccaccggttacccaggaggtaacagagactgatccacaatgttcctgaggtgtcttgggcacccataggcaggt |
| NO: 195 | gacttctggcttagggcagggttcaggaggctgggcactcacagtttctctttggtccccacagaacatgtt |
| 226972_s_at | gtttgggttgtggaagcctatggtattcttggctattgcagctgtggctctgtatgtgttacccaacatgcgacagcaggag |
| SEQ ID | tcagagttctgcctcatggagtgatggcagaccttggccagcgcgagggcagatccccagtggccaccaccctcagctttgg |
| NO: 196 | gcaggacacactgtgccagaaccctccccatatgttccatgtgtccccatctcctcagcctcagtcacccaggctgaaaagg |
| | cttgtggggagcggctgacttccatctcctgccttgtgtaagaacctgagt |
| 227073_at | tagcattagctttacttggagggganaagaaacaaaattacaccttaaantctattagatagactacaaaatggatttgatg |
| SEQ ID | tattcttacttttccaaagaattttttccacctggaataagaagagaggactagttganacatttgagctggagaggcttat |
| NO: 197 | gggcaatatgaagtgggccaaattcttgcaatacaccctattcaattgaaagtacagtgtctaaggataagaatacttcttt |
| | aaagaccaacaattgttttaatgcaatcttaattagtaaatggtttacattttttctattgttattggttttaaaatttgct |
| | accctgaattctgaaatattataagagttaataccaaaaatacttttaatgtctgacatctcttacatttcacatggttctt |
| | tttattcattgtaaataaaagtaaacttggataaatttgagaataaactttgaattgttgcaaactatgattatcaatttgt |
| | aaatttaccctttgacataataatg |
| 227119_at | agtgccatgtaattgtagcttgctagtgtttaatgtttaatagactggttctgtaggtgttttaaccatttaacactctctg |
| SEQ ID | ccatccctggagaaagtggttctactcttactgaacacattctctctgacaaaatcaccagctgctttatttttctatttat |
| NO: 198 | tacagttaaacagttgatgaggtctgaatcttgaccaaaactgctcagctgagatgtttttcacaatagacactgtacaaag |
| | tgtgcgtgcaaaaggacacggttggtagtattttttcattaatgtgaacattgactaaaaaaaagcagtcctgccttttaaa |
| | tcttgtggcagctcagaagggaggtgcttaagaaccttaactactatgtcagataacaaaatatttttttccattttggaga |
| | ttggttactgctcacacatgatgtatagggctaaatatatgcttgtttccttgcacctgtgtacttcccctctctccctccc |
| | tttccttcccctgtaggcaataaat |
| 227135_at | tcctcatttaactttacaaccttgcgaagtgggtccaggagatttggagtttgtggtaaagccagtaatgggcattgtcctg |
| SEQ ID | cattcccttcccttcatggtttgcctcgatcctctctaagcttctatcctggcctgaataactcaaagataattggtctcag |
| NO: 199 | agatcaagccatatcctcaggccttatttccatcttctcatgattctgccatcatacctttgcttctccgctaatgaaatga |
| | gctggcaagacctctgttcattgtgaagtgcttctgaaagagcctaagaaaaaaggctcatctgaaagaaatggagaactct |
| | atttcgaaccaagcctgtttgaatgtgtgttagtctgatctttgatcatgtgtttccatgtaatgggagtctcgttttttat |
| | aatgtttctaacgttttattgaaaaacctatggccctccttctttctcaatagctactttcttactgctttttga |
| 227165_at | ggacacccagtagaccctgaatttcactctctctaaaaggttctgagggctcatcctgggccaggggccctcctgtgcactg |
| SEQ ID | ttagctatggccacgggagcctccagagctgcctggtagcttcaggttgacctgcttatcaggcctacgatccttctgattt |
| NO: 200 | aagtacagctggaaagtattatctaattaagttcatgatagtgcttttggagaacttgtcaaattacagccaatgagaaaat |
| | aaggacctagcataccgtggagaaccattaaaaatttgagaagaaacaacaagtattatgtcaacttacttcaaaggcgtag |
| | ttttgggaatttgatgcagtaaagattaccctgttttatgattgttccttgaaagtcaaatgggggacctgtccattgtgct |
| | ctattaatcttgtcagaaaactgtcaccaaaacaaaatttgagtttgtccttgttctag |
| 227263_at | aactcactcattgtcaggtgtccgtggagtgtttttggcatggtgacctgtctgggcccagcatgttgcagatgtgtattta |
| SEQ ID | tgcgcaatggtatgcatatctctgtgtgactgtcagtgttgcaagctggctggatccaaccatctcttctgaaataatgcat |
| NO: 201 | ccaaagggttgatattctgggggaggtcactgcagaaggatggaactgacctttattccccagtgggcagttactgagcttt |
| | cctcctcagagccatgctggcagccctgggacagagaacggtgtggctttggctgcctctgcatggaatcttgccccggact |
| | cctgaagactgcacaaggaatgaggaagatcagggacaacctgggaactgaataactttcaaagccagtgctcagcttctct |
| | gctccgtactagcgtttacaggtcttaattcaaaccagatgcctgtactagtttttagacccca |
| 227265_at | acatctgctagaaccttttgccttaactattcaccaatatatgctaatattcataaatatggattgactgtttacaaacatt |
| SEQ ID | agaatcttgtcttggttccattttgatggctaatatttgttatcttaattaagactatttctgaggtcatgattacttgaaa |
| NO: 202 | atattgactaaaactgggtccttagaaattccaggtggagctgatttacctatgactgaggggaaaaaaaaatcaaatttta |
| | ctgataatagtaatgctccaaatgaattaatgacacatctgttcaataaataaagagcttaaatatacaaaacataagaaat |
| | ctgggcaacaaaacttgtggtctttacttttgaatagctacccaagaaaaggttttaaaggtaaaagttatgagtaatgtca |
| | tcacaataagctcttgtttaaaattcttttcttttatgtataattaggtttatgtttcatgtcttt |
| 227908_at | tcttcaccctacaacatctatgttaggtagaaaagtatgtggatttttaaaaatcatctggtataaagagtttttaagaact |
| SEQ ID | atcttcatcatctntagtatcagatngtgcttgagaagttaggtacaatgtaatgtgattcattgctaaaaacaaaatgcct |
| NO: 203 | cccacaattgcagactcactcattttaccaaactatggaatttgtcaacaccttttcttgggaccaaggaagcaaagtcagt |
| | ggataccgtgtcctgtagacatgtcacttgtgataaggcacggacaacatgaactggcaaaaacattggcatttggtcaaat |
| | gcttatgagctaaggatagaagggaaaaaatagttgtgtgaatgattttacctcttagttcttacgaaggatggttatcggg |
| | ctggaaaaatgtttattggactgatgctttggagcctctcaatgaatgtaataaaaaaaatgtgtagtgagcacaaatgtat |
| | ggatttgcagcacaatccaactttga |
| 227927_at | tacctctgtcgacctgtgaaactgaacaaacagcttatctgcccctaatgtgaaatgatgggacagacatagaataacnact |
| SEQ ID | acagtgattctagttcaaaatgagggaacatggaggggataaagaagtcantaacccaaaatagtttggaaatggagctggg |
| NO: 204 | caaaatccagcaggagtttcttagttaggatccacagcctgggactgaccctctgtcctgtgggtctttgcctctgggctct |
| | ctgctctgcatttcttgaaaccattatta |
| 227929_at | gagttcatgtgtctaatcattatctgtggtttgggggncnccattncattaaaatgaatgagtcactaagtgagtcactgta |
| SEQ ID | nataaaatacttggaaattgtaaagtaatatncaaaagatattaacaaagtattattcatggtaaacaaatcacatcagtag |
| NO: 205 | caaacacagggtgggaactcaatcttatgttatagcttactcttaagaagaatatgtgaagccaggaacatggttaaaggta |
| | cagaccagaaaatttctgacatgtgataaatatttcagtgacttttcagatttatttcttgttagccgctgtgtctatttgg |
| | tgctacaaaaactgaaagaaacaaaatccctgatgtaagggcttataataaatacacagtttggagatgaactaaggatatt |
| | ttacatttacgggaaaggattggacaatagtatcattttctcaccatttgcatgtacatcatgtctttccaattgttttcat |
| | tatttttctgaaagagctgcaaaattgtgcagtggttcaacctgaccaaagtggtattatgctgctgggaagtaa |
| 228017_s_at | gcctgcgtaagtgaggaaacagctgatcctgctcctgtggcctccagcctcagcgaccgaccagtgacaatgacaggagctc |
| SEQ ID | ccaggccttgggacgcgcccccacccagcaccccccaggcggccggcagcacctgccctgggttctaagtactggacaccag |
| NO: 206 | ccagggcggcagggcagtgccacggctggctgcagcgtcaagagagtttgtaa |
| 228054_at | aacggaatacctgctaggttccaggaatgagctcacctaacaganagcaaatgtgtctggttagatctcagcagagcccatt |
| SEQ ID | ctgcaagacctggctgagccagatgagagggtgggccctgtgctggggggnccttgggtcacacacaggaaccgagacctgg |
| NO: 207 | cttccaccccccagtcacccacttgggttatctgctggaagttatcgataggactgtgtggccaaccaagtgcttgtgagat |
| | cactgacactgcaaaaacaaagcaaactgctccgggtaccaggacttcctccaacctggcaagggtgtgcgctgaggcgggg |
| | cttgcaggtgagggggctgtatgcttcaggaactaactaaatgcatgcagaaggtaagaggcatgatgggaggtgttcaagc |
| | acagcaatcccatttgggagttattttgatactgcgatgagtaagggtaagggcgcatggaatggggcta |
| 228071_at | aggagttggtggagctgatagagaaaatggtgcagtgcaacgaaggggcttacttttctgatgacatatacaaggacacaga |
| SEQ ID | ggaaaggctgaaacaacgggaagaggttttgaggaaaatctacactgaccaattaaatgaagaaattaaactagtagaagag |
| NO: 208 | gataagcataaatcagaggaagaaaaggagaaagaaattaaattactaaaattaaaatatgatgaaaaaataaaaaatataa |
| | gggaagaagctgagagaaatatatttaaagatgtttttaataggatttggaagatgctttcagaaatatggcataggttttt |
| | gtcgaaatgtaagttttattcttcctaatttactgtgatttgttaatggatgaattgtattttgcaaagatagttagagaaa |
| | tacctccttcc |
| 228094_at | gggagaaacacatttactccccaataattgtacgggaggtgatcgaggaagaagaaccaagtgaaaaatcagaggccancta |
| SEQ ID | catgaccatgcacccagtttggccttctctgaggtcagatcggaacaactcacttgaaaaaaagtcaggtgggggaatgcca |
| NO: 209 | aaaacacagcaagccttttgagaagaatggagagtcccttcatctcagcagcggtggagactctctcctgtgtgtgtcctgg |
| | gccactctaccagtgatttcagactcccgctctcccagctgtcctcctgtctcattgtttggtcaatacactgaagatggag |
| | aatttggagcctggcagagagactggacagctctggaggaacaggcctgctgaggggaggggagcatggacttggcctctgg |
| | agtgggacactggccctgggaaccaggctgagctgagtggcctcaaaccccccgttggatcagaccctcctgtgggcagggt |
| | tcttagtggat |
| 228128_x_at | gatgttgaaacctgtttggcaccttctggaagctaccaaaaaaatgacactccattgaagtgcttaaaagctgttctcataa |
| SEQ ID | gaattctactggcctattgtaaaaaagaaaaaaaaaangaaaaagaagaaagacacaaagaaaataatctaaacaccaaaaa |
| NO: 210 | ctaaacacaattccaatcctttttctgtacctcacgcgcataaattngctgctcctattttttttnctgttnatgtgttttn |
| | atggatctaagttaaatcttttggcaatatataaaaatgtaaatagtaaactttatttattaagaatgtcatcttttttaat |
| | ttatatttacacaattgttcatctaatttattttttctatacagttttaaatactcagacatattttgctgttcatgatatt |
| | tttatcctgttctcatggatttgttttcccatactgttttctctgatctcaattacaggttggatctcacaaataataatgt |
| | cagagacagaaatattttgccactgttgattactatac |
| 228492_at | aaccaatacacaaaattttcctatgtcagaatgtggtggagcataatagattgtatttggtgtgcttgcgattttttttttc |
| SEQ ID | catagaatttattaagtgaagtttctaaaactttgcttctcctgatcccggtgaagtgtacatcataagaatccatagtact |
| NO: 211 | ttgaagtaccattgcaccaagatgtctgactgaattcatagtcacacttttatttgaaagaaagaattgttgtagttttttt |
| | tcattattctaaaactcttgttgttagatacaagatttaattaagatctaagctcctgcttatttaatgtaattctaaggta |
| | ccattttagaaaaaacatttgttttaagattccaagaaacctgtgagttaatactatatttaaaagagaattggtaaatttt |
| | gaatgtgtgtaatattttggaacctgtttaaaaaccaaatatacctgcaaatagatacagcctatcctatactattta |
| 228565_at | tatgtcctcagttttacttctaccaaaacatccctgtatgtgtgtgcatgtatgttggcgtgtgtgtgtgtgcatgcatatt |
| SEQ ID | agtaaatgtgtgtttgcatgtgtgtgttggggagtgtatgtgatctgggtgtttgtttatctctgttattattcccctttag |
| NO: 212 | ctttattttagtcaactctacattatgatgaatttcaaaatgaagctgtattaaaataattgtaatataacaattcaatctc |
| | acatgttactgcagatagttaacttttgctgcaatctattgtacatttgcaattttctgtgttagtaaacttagcagaatct |
| | ggttatttatttttgtgtaggcttaatgttcactgaaagataagtcaattactgttagtaaaaaattaaggtactctcactg |
| | cagagatttaaggcctgggcctaatgtgctgtattatgaagccttgtgac |
| 228604_at | gaaatggtggtttccttctgataagggatacagtggaccatgtcgcttatatatcaccctactaggggatataattttccct |
| SEQ ID | tctaagcaagggaaatggcagtgctaaatagttttgtaaagtttttgaatgagaagcttttaggtaaagataaactgtcata |
| NO: 213 | atatttttcagatgcatttgcattgactatgggagaaaaattagggggaaatgacagtcatggtgtttttttttaattttaa |
| | ggactgaaattctgttacatgatgtatgacaatataaaaacatacttagttttatactaaatctttttttaaggtcttggca |
| | tttaatataaagcaaattcacacgttttctaactttccataagttccaaaaagggaaagaagaaacctcagtcttgaaattt |
| | tgatttttaaaaatcatgacactgttttaccatgaaattgagtagctaacttttggtaacacct |
| 228812_at | gagacttcattggtgatacactcaatttttactgggtaattagctaataatgttggtcactgtctcacagttcaagtagctt |
| SEQ ID | taagatgatgtggcaaggaaaacacaaagcttttgggtaaccagcgttcttaaatgtatggtttttgaccaggtgaaccctt |
| NO: 214 | tagaagtgatttctgttttaaaagtatgtacttaaaatacctttggctgtgatgaatgtagatcccagcagaataccaaaat |
| | cctattttttttgactgagtatttgtagatgcttaatgactgaaatgaatttggaggcactgatgaaagtgatttttttnaa |
| | gttctcaggtactgttcaattat |
| 229022_at | gaaacacttttagagcacttgaggaacctaaaagatgactggttcagcattttgtgtggtagataagaaagaaattatcaca |
| SEQ ID | aaaaatcagaaatgaacagtgagagaaaaataggaccccagacagtttataccttccatttgctgttttaaaagtgtgagcc |
| NO: 215 | tgccaagtcaacaagtatgcctttagcgcacatgtaaatagcctgcacttcctaaatctcgtgtggcctcccatggttacat |
| | tcttcaaaggtnaactgagttgagaggaagattcagcatttaaaagagaagggttgaaaaagatnntgtgtgtgtgtgtgtg |
| | tgtgtttaattggcccagggttacttaaataaatcataaccattttgccacattctgtaactgtttagctaaggtcaaatta |
| | agtttaccctatggattttgtttcatcttttgtttcgtgtatatactgtttgcctttttca |
| 229026_at | aaaggcccactgataatgctgcactggaaatgctaaacccaatgttaactaaagtcaanatttnccgctctacgcttactgg |
| SEQ ID | caccacttcatgcctacatcatgggaaagtacctcttttacagaatgcagatcactgctaacaatccacatgactaagtctt |
| NO: 216 | cagtttcccttgcttaacatttgttagcatatatttcagcaaagattaacagtataatgaatgttgagtagtaaaatcctat |
| | ttaattcgcttcaatttaaaaatgagatatgctgctatggggaacaaatgacctcttgagatttacaaccccctttataaaa |
| | gtgggaaagctgcctccctaaaaaaaattatgtccaaggttaataccgtatctttttttgtagttttcttatttcttgattt |
| | tattggcacctcccaggcactttggcatgtgctcctgtataaagtgtagttactgtgcctgtagagttagtggcagctgcag |
| | agtagttataggctgacaagtccacttttggcttaccatggagatctgctccaacacatgc |
| 229047_at | cctgtgatgatagcttcttcccatgtggggattaagggtagggagtggggggaatggtccaaacccggaaggagctaatgga |
| SEQ ID | gaggatagggccaaagtgggacagggcttggatggtagaggatggggtccaggaggaagaggagtctagcaatccaagcgca |
| NO: 217 | ggggtcagtgctccgagtcccagggctcagaaccagcagggcgaccacatgagcgagcccagcgccgccccagtggcggntc |
| | ccgcaagcatgcccatggccagaggggcgccggcgctgtagcagggatcctcccgcactatcacgtgcgtcacgccagggcc |
| | tgcgtagggcggtccgtagtagctgcggacatacgtggcctcgtgtgcattggggggcaccacctcgtagtagtcttggtac |
| | gggctgtagacgcgcaccggggtggagtt |
| 229094_at | tcccctagtgaaatccatctgtcctgaagggaaccctgggacggggtcaaggaaaccctgttcacccccaccacggggtcag |
| SEQ ID | gtcttcaccaaagggaggccgcacggattcccagtcgcaaacccaaagccttgctgcctttgcctctgcttnccatagttgt |
| NO: 218 | ttcaatgtcttttttaaaaatcacatacactcgagtaagactgagtctcctcaccgaagagatctggctgtatcccgagtct |
| | ctgaaacctttgctccagtccngtgggancgtgccgtcccgaggatgtggagccgcaagggctcgccagagggatctcagga |
| | gctaggcctcaaggagcgctgttgagtgacaggtctgatgcagtagcttttactattggtggaaatcgatgttttttccttg |
| | aaagtctacctttctaagttcagt |
| 229308_at | gagccataaaattgaagccagtagcttgaacaatatggaggccttcttactgtagggaagaggtgtctagcttagaaggatc |
| SEQ ID | acatttgatctagatgagattacccatagcagggaaaatgaatcatcacctaccaaagactgtacggaaaggtgtgttattt |
| NO: 219 | tcaaaattgcattttagtttgcacacatgtaaatgattattctatgttaatctaattgacaattcagttagctccgcatcct |
| | aagacttaaaactagcaattcctatgcaaatcatctgcttaactgaactacaaacgtttttcattaattcat |
| 229575_at | gaaggctatgagttgctgctggccccatcataatcttttttgtttagcagaagtcaggaaacgaatactggtacacttacat |
| SEQ ID | ttcttggaagagcagcagaaaccttgggatatccttgtgggtcaaggaggcaactctcctcacggattccctgatttgataa |
| NO: 220 | caacatactcatggaaacaccaatccaagttttcacaagtttctcttaaagttaacttaagacttcctagaccaagaattcc |
| | taccacgcaaggcctgtgaaaatattgtaaacttaatatccgacagggagatattcaaagatgccaaagtacctgtttttat |
| | aaatattaccacatggtttaagatttgacttctcacataaaatgagtaaaaataacaatgatttttctgcagtaccttaatg |
| | atgctgtaattatgaagcagggttttaaaattctgccatgtagtgccttattt |
| 229598_at | atttttcttagctaaatctggcaactgtgnctatttcattgaaaacctgaaagtgtacaaagaaggaagaagcagaatctgc |
| SEQ ID | catatgagtaatagaagtgagcaggcccaggactccctaagtcaagaaaccaagaggcgtcattacggaaaagagtaactca |
| NO: 221 | ccctgtgtgctccttggtagttctccctcagcgatgcccccatgttatgaatggggaaaagttcactgaagggttcatagtg |
| | aagaaactttttggatgatttctgttggtgggttttggataccttcaagggatcagaaaataatatacttaggaaattttgg |
| | taatgtcatcattactctctacattattattatgacggttacaattgttaaatctaggtggtgggtatgtgggttatattgt |
| | acatgatttttaacttgtctgcatgtttgaaa |
| 229623_at | caacccaaagtctgaatttcttgaggaaaaggtcaatgtcttttcatctctgtattgttccccacctcttaacacagtgcct |
| SEQ ID | ggcacatactaggtgctcaataaatgtctgttgaatagaattattaataatttaagagtaggaaaaaggagtgtcagagaga |
| NO: 222 | gactcaaagatgagtgagagagcatggaagatggctgtttcatgtgagacaggaatggaagaagttatgaatagggatcaga |
| | tgggggagtgagaatccctgtaagtaaaatgtgaggaaaagaacaagctgagaacaagattctcctgtcaaaaaggcgtctt |
| | actgtaaatagtccaaggtgacatttgttagttttgaatactgcttttgggtttctttttttcatttttatattttaaaatt |
| | tttatcaaagaacaaagacttatgtagttttcttttattctacacaatccaaattaaacagcttttggtgatgcaattgtac |
| | actttcttaagaatatatctaataccacattttagcagaaccaagcaatgactgacattattcattgggatctggcc |
| 230164_at | cccagcctccaacctaatttgagtttcatttgtatttttttcatcaccttacatgtgttaacagctgtttcaacataaactc |
| SEQ ID | cattataatctatatacatttaaagaccgtgtttacataatctcttctgggtagaaaatggaaatacttaacctttgcattg |
| NO: 223 | gtccctttcaggcttgaacaacagtggctgtatctgcatcgtggggttgctggtggtagaacactttggtgttaggaaagtt |
| | tggaaaagaggatatccctgtattaggccattatgattagagataagcagcctgggagaaacatacaagacctgtggctact |
| | ggtagagaattggaataagcttccaacaatggactgtagagattaatggagattgtttagtgggcccatggctaggtggggt |
| | attaatacagttgaagttatgttncccttggcacagatctttgagaatc |
| 230330_at | gggcctctcacaacatggcagttatttgtccccagagcaagcaacaccggagggcaaggaagaagccatgatgttttttgta |
| SEQ ID | acctagcctctgaaagtgtcataccaattctgtattttgttggtcacacagaccaagtcaactacaacgtgggagactccta |
| NO: 224 | cacaaggcatgaattctaggaggtgggcatttttaagtgtcatctggaaggaggctgtcacaacctggaa |
| 230521_at | ggcctcctcccaaggagaacatgagaccacttcatatctggaactctagaaacactggctgcagcaatcaacctcctgtgcc |
| SEQ ID | aaggctggccccactcttgtagcatggacactgctagctgggttcaaatcccagctccagcagttgccagcgctgggtgacc |
| NO: 225 | tcgagcaagtgactactcctcactggattgtaggagaactaaatgtgatagtcattatttacagttcaccacacgctcttca |
| | catccgttacctccctggatcctcaaaataacctca |
| 230760_at | aaaacgtgacactttacatatttcatatttttcatatagtctggaggtatacacagttgttttgtttttaaccacggtattg |
| SEQ ID | aaacctttaaaaggtaattaagcatttggtcaagtaaatatagaacatttgtataaagaaagaaatgaaattgtgcttaatg |
| NO: 226 | ttatatactgttattatttgcaggctggttttaattcttaatttgattagcaaagctaaaaaagtggatgttgaagttgaaa |
| | gttttaaagaggtacaaaatcttacaaggacataaattattatttggttgaaaaatagcctattagatattgtatgtccctt |
| | cctctgtacaaattgtaaaatacttaaaataactatgaagaattcatataggaagtgtgatactattgtaaat |
| 230968_at | gtgattaaacattctagaccaggcttgttgatatgtatgccaatagnctagaatttttggcttagtgtaaaataaaaatgtc |
| SEQ ID | ttttctattgtggtctgatatccgtttctgtaataagatcagtttgttgtcctctgtgcaccagtggttttgcccttaattt |
| NO: 227 | tttttggctagcatcaccaagatctgtcatccagagctgctgagaaaaatacatgttgnccaaacttttcttaaaattgtgc |
| | tgccagtggtattttcccagatgtgaaaaataataatctaataaaggattaatatctaataacaataccattgttgaacatg |
| | ctcatggaatgtccaccttcttctgattccnttttttgtatttgaaaatgcaatggtgtgttccaaattattgttggtgttg |
| | ttaatgtcatgactctcctttgaatagaataaaataaccccttttgttttgtgttttctactgaattagattttcctctagt |
| | cctatgtgaat |
| 231084_at | atagcaaattatgccctaagctgcaatctacgagaagagttggtagctgtctcagagagaaaagacatctgtaatgcaatgg |
| SEQ ID | ggtctaaactgacttgtgaaaagattgtcaaagaacggtatgaaaacatgatgcaacagcagaagttaacaaatatttcaaa |
| NO: 228 | acaacaagctgaacagatttcaatactacagactgaagttgaaagattaagaatgaaaacatttcctgctcttgttcaaatg |
| | taaaaacgctggcaggaaaacacaaggccaaatcaatcatttaaaaaatcatttcatttgggtaaaatgatttccttttctt |
| | tcttactgggaaattgaagtagcatgtcctctgagttgcatgttttagataaaaaattgtttaattttagccttaattgtat |
| | acaaatttttgtcctgcatgaata |
| 231094_s_at | ggctgaggaggctggtctgaacatcactcacatttgcctccctccagatagcagtgaagccgagattatagatgaaatctta |
| SEQ ID | aagatcaatgaagataccagagtacatggccttgcccttcagatctctgagaacttgtttagcaacaaagtcctcaatgcct |
| NO: 229 | tgaaaccagaaaaagatgtggatggagtaacagacataaacctggggaagctggtgcgaggggatgcccatgaatgttttgt |
| | ttcacctgttgccaaagctgtaattgaacttcttgaaaaatcagtaggtgtcaacctagatggaaagaagattttggtagtg |
| | ggggcccatgggtctttggaagctgctctacaatgcctgttccagagaaaagggtccatgacaatgagcatccagtggaaaa |
| | cacgccagcttcaaagcaa |
| 231229_at | gcacgtccaaggtgatcctgagggctgtggcggacnaaggggacctgcaagtatntgtccctgnncaccctgaagaaggctg |
| SEQ ID | tttccaccacgggntacgacatggcccgaaatgcctatcacttcaagcgtgtgctcaaggggctggtggacaagggctcagc |
| NO: 230 | aggtgaccggcangggggcctcaggctccttcaccctgggcaagaagcaggcctccaagtccaagctcaaggtcaagaggca |
| | acgacagcagaggtggcgctctgggcagcgcccctttggacagcacaggtcactactgggctccaaacaggggcacaagcgg |
| | cttatcaagggggttcgaagggtggccaagtgccactgcaattaatgaggcaggccaggcaagcagtcaggggtgccaagan |
| | cgccattggctcagtgcagtgggaa |
| 231592_at | gtgtttttgttgcatctccaagtcagaataaatgccttttggagcagatatatttcatttagcatttagtatcatcttcatc |
| SEQ ID | aatactcgtatgaacgaaaaaataaaaagccctctcttattcccactctacaacgcatgtcaaaggtgatctgtttagtttt |
| NO: 231 | tccttagtatcgaacatatcacagctactcaatgaagtttcttctcaactaaagaaacacagtctcttagagaatttgttcc |
| | tgtgtttccaccataagataaatgagataaagaataaatttttactaaattggtaaatgcaaaagaatcttttttttattgc |
| | tcatatgtcttcctgtctctaagccagaccaatgagcaaatacctttaaaactagttgttacatcttgaaccatttaactgt |
| | aataaaagcagaatgtttagttaatgaattaaagaacaaaccctgagcccttttatcagtctcctggctttaaactaagcca |
| | atgaggaagtgatttgggggattcctgaaacta |
| 231715_s_at | caatgcagttgaggcctcctgtatccgaacacgagagctacagtccatggccgaccaagaaaagatctccccagctgccctt |
| SEQ ID | aagaagaccctcttagacagagtgaagctggaatcccccacagtctccacactgaccccctccagcccagggaagctcctca |
| NO: 232 | caagaagcctggccctgggaggcaagaaggactaaggcagcatctgtccccntctgtgattcagagccnttagttgagancc |
| | cctgccgcccctgccacccccctgccccgctcccaccattgcccctcctcagctgtgcaaggagaaagcatgcttaggaagt |
| | tttcaggtccttgtgataaaacctccttaaatctgttcagaccaagcaatgcgagc |
| 231890_at | cattgtgccattctagacttacctcctgccactccccaccagctttgttttgtcttagccacacaaaataatctagcgtctc |
| SEQ ID | taaccagtcaaacattttaccttgtgccttggctcactctgtgccttttctccagaatatctttctgtgtacttttctccca |
| NO: 233 | tccttttacctttaaacctgctgctatggtttgnatgttgtttggcccctccaaaactcangttgtagttcaattgccaatg |
| | taatagtgttgggagatggtacttttaagaggtaattaggttgctaagatggattaacatctttctcttgacactgagactg |
| | ggttctcctgggaatggttagttcccaagagagtgagttgttataaaacaatgctgcctcttctattttgcgctttttgttt |
| | gcacaaactcggtccccttctgtttctctacgatgttttgatgcagcatgaggcagtcatgagaacccaccagatacagctg |
| | cctgatcctgaatttcc |
| 231929_at | ggctcaaataccatacctcagaaaatgaggtttactatggaaatactgaaacagtctttgcagctgtgtgacaagtcactct |
| SEQ ID | actacatactgatttggagacctccgctaaatagttttatcactgcagactaaaatgtgggacttgtatcttctttgttttt |
| NO: 234 | aatgcacacacatacatgttctgtgcatgtatgtggttactgngtatatgtgtatgagtgttgtatatgcatgtgtgagtgt |
| | gtgtctgtatgtgtgtacaactaaagaagctgcagaaactttgtaatactttgtgaaaaggattatattataaaggtttgta |
| | ctgtctgagtgcacagctactggaataaatttagggaatctcaggaacaagcatanaannnntccaagatttatttcttctc |
| | agaagtgtaagtgcagtttttaattctgtat |
| 232114_at | gaactgcagtggaatcgtccaactatttattgccagttttggttctcctgaaccttatgccaccttaaggggaaaaaaaaat |
| SEQ ID | ccagtagctggcttgagattccagtgctcacacttgacatggtttccagagaatctggccccaaagtccagaaggctctggt |
| NO: 235 | tttcataaaaggtgtatttgctgtttattttgtatggtaagtatttgctcttttgaatttaattattactgtcagtgtcagt |
| | cttggttgtgtattgcatatactgtatttataaattggtgcaaaaagcacaagtaaattatacatcaaatttattataaaga |
| | aatagtaactattttaactttgttcaagtatgtggtaatttgctcctattagagtaaaaaagaaaccagtaaattatcagtt |
| | tgtgtaacttaagagnnnncnannnnnnnnttttttagatctagatgcataaaattttgaatgtgaaaattgcagggcattt |
| | taaaaccatatgtgggggatattttcccatgttctctgttatt |
| 232204_at | acagcctgcaagcgatatctggcatgattgttcctcctatgtgaaagaattgccttgaagaattgtattaatgaagaggttg |
| SEQ ID | gattctgctacagagagtaatctgatacaagtcccagagtggaacttttaactcaggcctttttaagaggaatcacaataac |
| NO: 236 | tgcagatttttaaacaaacaaaatcaccgaccttgcaaatactgaaattggaagagggatctgcaagtgcagggtgttggtt |
| | aaagttgtacctcccaagtatttgggggatatatttattctgtattgacaaaagcaaatccactttttctttttcttttttt |
| | tttttnaagcttaattctgcaatcatttgtcttttataaaccgtaaagctctatacaagggacactat |
| 232618_at | agaaggccgtgactttataacaaagaacaggagttgggtgagcccagtgcactgcacacaagagtcaagaagggagcttcct |
| SEQ ID | gagcaagaagtagcccctccgtctggtcagcaagctttacaatngcaacaggaacaaagaaaaagtcttaggaaaagaagtt |
| NO: 237 | ttattattgatgcaagccctaaacactctttcngactccagaggagaagctggcagctctctgtaagaaatatgctgatctt |
| | ggaaattcacctcttctatagaagagtttgttttgaactatacgatttgaaacaaaattctttttttggagactatggaaac |
| | attctcaacagggaaaccctactagactttgtaaagcaaataatggaaaagatacagaactttttgaagaatcatgggaaat |
| | ttttataattaaataaatgctaaaattctgttttgtgaaacatttatgggaattatcactgacagtttttgtacactttcaa |
| | atagtgttaaagcagcaactccatgttgtaaatgcacaa |
| 233976_at | tgagtcttttgaacacaggtatgaagtacaaagtgaatctaaaccaatgttacccctaccatacaggtatgttgtgttcaca |
| SEQ ID | aacacacacacacacacacacacaaattaaacatggtggaaatttttgacaaanttaaccacattaaatattctgtccatta |
| NO: 238 | agattccccaaagatggtgganngatngcagngttagtgngagaagatatttgcagcatgcaaggcaagatgacaatgcact |
| | aataacaaaatttcataaaacaatggacttggggcttgggaaaaataattatgacatgctttctgaagtgtccccagatagt |
| | ttcataaaaaatggagcaaataggctaaagaaaaacccatagacatcatgttcaaaagaactgatgacaaggcatgttcata |
| | acccataaaatacaagtggatgcagctaaataactgcaatacttttgaaagagtttggcaggtttcgaagatgtg |
| 235048_at | tcagtatttttattcgccttctagaacttgngatccacacatcacccatttatattagtggtaaaattatctgctaaaatct |
| SEQ ID | aattgtaagaaaggcttaccttctgtcatcaagtgattgtatcatcctggatcgtcatttccaaggaactagcctttctttt |
| NO: 239 | cctaagcgtctgtatgtgttctaaaacttccagtatatatttatagaacttagaaaaaatgttacattattcagagtagcaa |
| | gtcttactggagtacctatgtttgttttcttggtttttatttttttttaagtttaaaagtagtaattaaacctatattttgt |
| | gattgtttcctggtctgtgtttttaaattcntttccttcagttttcctcatgaagatgtttcagatactgaatttgtttaga |
| | cattgaatgactttgttaaaggcacaattaatcacattggttgtactctgaagac |
| 235092_at | cctttcaaacgtttaccagctaatgggcaagnctcaaggncaactgagaaaggggctgcgagtggtaatataaacaccaaag |
| SEQ ID | ttctctaanatacacaagacctgtaggctatgtgtcttctngtacttcaggtgtgtagtctatntctgacaggttgtgttag |
| NO: 240 | tgaattttaagaatggagaggagaatttagggtttgctttttctttgaaagcaaaaataaatgttcatattcttctgataca |
| | gtaaaaacccttgaaatgccctaatttagaatagcacctgattaattaagagttaaaagtctaagaaagtttattaaggcgt |
| | gcatggccttgggataacaatgaaaccatttgtctactggcatctgtctgctggtttcttgacaccagtgtaatatagaatt |
| | gtgacgtaaagaaccncaaaatataagagggtttcgtttcattggttttgttttgttctaaggaatgcagtctttcagtcat |
| | tatctttt |
| 235310_at | cgatccttggagatcccgtaatcccactacccttctttggctcagagaggataatttgcctaatgatacattaaagttagtg |
| SEQ ID | gcaaaacttaatttggagcctgatttcctactgacttccaatttagtgctcccccagtatgctaaatagaaagccctctgca |
| NO: 241 | atatattaaatgtatactaaatgtatatatttaataatgtcatgtataaaatatgaataaaatgtccacataggaaattaac |
| | acatatataccttctctgataagcactcctcnnatgtgtcctgattatttactttctcttatcttttccttagtgttcctca |
| | aattatatctatcctctaaaccagggatcagcaaactataacccccaggccaaatctagcccactccctgtttttatagata |
| | aagatttgttggaacacagccacactcatttgtttacctactatctatgattgttcaaccatgggagagttgagtaattgtg |
| | acatggaccataggccctaaaga |
| 235569_at | gcctctagggtgatggaccagccccnttaaagnacttgactcaactacaggggcctgggaagatgcctgggtcccctagggg |
| SEQ ID | ccttgccaaggggacctgtcgcaccccaccactccactgggctcgcacaacgccaaggccgccaggagtgttttacatcatg |
| NO: 242 | tcctgagcctacctttcccccaaattctggggcccacagcctaggagccaggtgatcaggcctcggctgtggggccagggac |
| | accatggccctggggctactacgtgtccacacatgctccagaccctggggcaaggtaggccaggggcttctgacctgtgcag |
| | gtgagagtgggccatacccaggaaagaccattctgtatttttctgtccctgtctccttagaatggaagctttttgagggcag |
| | gtccttgtctttgtacgttctg |
| 235885_at | aaatgtatatatatcctagtcccctaaccaaatcctgacctattgggatacttataaaaatttaagtaagtgggatacacaa |
| SEQ ID | agaataataactattaacttttcattattagcaaaaacctaagggatttaaactaattgaaactgtatttgattggacttaa |
| NO: 243 | ttttttatgtttatttagaagataaagatttaaagaagacctttacaataaagagaagaaatatcgaagtcattaaaataag |
| | gagacttacttttatgacattctaatactaaaaaatatagaaatatttccttaattctagagaaactagttttactaatttt |
| | ttacaacttcaataataccatcactgacacttacctttattaattagcttctagaaaatagctgctaattaggttaatgaac |
| | attttaccttagtgnaaaaaaattaattaaatatgattacaaagttgcacagcataactactgagaggaaagtgattgatct |
| | gtttgtaattacttgt |
| 236694_at | attagagctatatcattttactctgaatgtcttaacatagaagtttacataaaatttacagattggattgatttcagccttc |
| SEQ ID | ttctggtactttttaaaatcttattaatcattaggaaaagaagttttattattgatgcaagccctaaacactctttcgactc |
| NO: 244 | cagangagnaagctggcagctctctgtaagaaatatgctgatcttgtgagtatttatttaatggagcaaggaacacagaaaa |
| | taaaatctatgtgtgcttgataagatttttaaatattattttgatgtaactttaaatgtaaaatgatattttatctcaaaat |
| | tgaaaacaatctcctttctttagtacttatgattggtgtgtgtgacttcatcttatgaa |
| 236707_at | tccagggtctctactgttctcctggccttttctcatgctggctacttcaggatttgagtgtctactacacctgtaggtctca |
| SEQ ID | tattttcaacaaagtacaaagaaagatgtagggatgatattacccgtgtctgtccctttttatcaggaaaaccaatgtttct |
| NO: 245 | ccagaaactcccctatgagaccgggtnccatggccatccccagcagctgatcataattggcttaaacaaattaggatttatt |
| | gcttggggttctgttagcaaaagaaagggataattatctgtattaatagacagttaataatgtctnccatagggagtaccaa |
| | acaatgacaatgacagaaaccagttttcgagtatatgcactgtatcactccctccccaccactgtccactcactctccacta |
| | gacctcacatccaggtattagcttctatgcacagagacttacctcttacctattcaactttaat |
| 238106_at | gggacagacacttgtcattgccaagtaagtcacatttaaattccctcaagccttgagaaataatgtgagggaaagtgcaaaa |
| SEQ ID | atagcacagtcattcctttggactaagtcaactcctaggcctcctgagagcactgtagctgtgtgttcatctntatgcaaag |
| NO: 246 | agaaataaaaagaatcaactatatttgtcacagggtactttcctatataccagcgtacacctctatcaatacattttatgcn |
| | tgagactttcggtaaggaaagactgaccnatacacactgcctcatgcacagatatagacacatattgaagatactttctttc |
| | ccctgcaatgcagaaaattgcatgagcacacacaaccacactaaaccaaccagaatgaaactagaggcagctgtatctgatt |
| | gaaactggtgagaattgagattgacagagttacagggctaggaggttaatgagtccatactcctgcctccagtcagaactgt |
| | cctaagc |
| 238693_at | gccctgaaagctttatagtcctggacatcaaaaattttatttgagatgatgaatgttttgttttcatcttttcttatattac |
| SEQ ID | cacaattgagatattttagtaattgaaggaacatacacagatatttggcagaagtcgagtaaggaggggaaaaaaagagtcc |
| NO: 247 | gtgagtttcagtcattttcactgctcttttcaaaaagattgtgttgagctngtagaagactaaagatgtcactgaagacatc |
| | acagatactatatttatcttttggctttgtgtacattagagaatgttgattatttttatacaaaaatacagcgggtaatttt |
| | tttaatctttanangcctcttgtttgaatgtatgcttngtggaattctttgtgtagtaatgttttaaaaaaagatgtttact |
| | gatagttacatgtaggattagaatatgtaatataatataaggctcatgttccagacctacgatagcttgtagtctatgttac |
| | gtatttctttatatcac |
| 239146_at | tgatctccaaacccttattttttctttgaactgtnaaatttccactcatggacgatgcaaccaacagatgcaatctctgaga |
| SEQ ID | agatgaaaattgggacctcttattataaaattgacctagctggactcaggaaaccagggaagaagtcaatgcaggcatttaa |
| NO: 248 | aatgtaaagttttttctggttaaatctatttatttttcttgtaggttgagtatttcttcccagtttttctgctctggtgtat |
| | aacaaacaggtcaaaatttcccatctttcctcctgatagtagttgaatcctaccttgcatacttaatgcatagtgaaatggc |
| | atctagcagaaatacacacccccaaaacacaccaccatttcattaggtgcccaaaaaattctgtatttagcttatttattta |
| | ttgttatttttgctttttcttaacccactatata |
| 239486_at | taaagttgggcagtaatcttaattatgatggaattatcattatgctaagtaattaactttacctagtttgttttacaactag |
| SEQ ID | aacctgccctaaatgttgaatatcttcctagcaagaaacagtctgtcattttacttacacgatgtctaaccaaaccataact |
| NO: 249 | ttacataaactagtcgtttcggtcaaatagaaaaatgtgtgaatgccataaaaacaaaaattctcagttaaatgatactggg |
| | aaatagggaagacagcaaagtgagacttgggctcanggatgngttcaggaanaaaaaaaaagaaagacccctgagtaccatt |
| | aatattcctcagaaattattatttcaaaaggaaatatttctgtattataaatttttcatgagcagccattatgaaatctcac |
| | aa |
| 239499_at | gtgctggctgggaccggaagaactcctgcttggtggangcagagcccatgcagcttgtctgcctcatgcccacgatccactt |
| SEQ ID | ccggcctgcagagagccgcaagaagagcgccaagggcatgtactcctgcccctgctattactatcccaaccgggcaggcagc |
| NO: 250 | tcagaccgagcctcctttgtcatcggcattnanctgcggtctggggccatgacacctgatcattggatcaagaggggcactg |
| | ctctactcatgagcctggacagctgagacctcctcctnttctccgcttgagagagagggtcagggactccaggagctaagac |
| | agatgttgcacctaggactgaggccggacctcactcagactttgaccttggccgaatttgtgtgatgtggccctggagatac |
| | ctagtt |
| 239726_at | agagtcactacccgagttattcgccggcgtttgattataaaggtatcctgtagctggatcattatgtggcattgctctacta |
| SEQ ID | ttagaaactccttccttgtggttgtagaaactccttccttgtggttgtgaaatttcagcatgcttgataatgaaatggcttt |
| NO: 251 | gtgtaatttttttgcacccttgaaagcttgtaaaaacaacaaccaaaaagaaaagaagccatgttgcatggtattctgtata |
| | ggcctatttgatatgtggcggttgtagttttatttganaattcttggcatttgtatcctgctttccttgaagaagtgttatg |
| | tgaatgagcaaagattctgtcttggtgacacagtagttcatatcaaatgaaatgtttaattgaaaccttagaaactgtaaca |
| | aggtatgaggcacgataaagaagaaagagaatgaaagaattaaaaggcttccttctcagtctcaaattcctactgccttaag |
| | aatttttaaattaaaaccagttcttcataagcaatcagt |
| 239834_at | aataactgctaaggtcctattttgaatttgaagactaagagggagggatgttctttatttcttatagatggaatcaaatttt |
| SEQ ID | tataaacatgaaaggagtatagcgattgtaacaagacttcttgccatggttatttggagctattaagattaactgcctttta |
| NO: 252 | aagagtgggtatttttctgattttcaatattagtttgtggctacattcctttttctactgcaagaatgtttgctctccttta |
| | tgaaaagttcaagttggtggagaaactaaatgttntgtacataagtatataactttcttcattcaggaagtagttactgagc |
| | gcctactgttttggcttttgtaccaggagtctataaattaatcgttttccacctggcaagtagtctcattggcaaa |
| 240013_at | cttgcgatgcatcttactctgtttaatgaatagtagatgcatgtctgttttttgacggcatctaaaatatcataattattat |
| SEQ ID | cttgccatttgtgcattttacacaaatatttaataaaactgttattagccaggaagggcaattcccttttctgatattgcta |
| NO: 253 | gctgtgtttcttgggttgccactattgccactgaatgaaaaaaaccttttcttcacctaaccaggtaatctgcagttctcat |
| | aatcgctttgtgctattgatgagcagtcatatgtttttcaatgctatttataaagtacggtaaaatcgatgaggaatatgat |
| | gctttcgattgttatactaa |
| 241671_x_at | attctgaagccagaccgtctttcctcacagcnnnnnngnnncnnnnccgngggcacgcaagcttcccctggttacctgagct |
| SEQ ID | gctcctgccgtctcccgcntgggnttcgcngtggtgcacccgatcccggnatcgtgcgtntgcgccctgcgaaagaaggncc |
| NO: 254 | tgctggcggantccggncggggtctcctgnctcgcagctgggcgaggggacttggaggacagggtgaagctgcagaagacct |
| | ggggtgggatggntagagaggacgccaaggactggggaaggggaagttaggaataccttacatccaatgccca |
| 241956_at | ctgcaaagcagtattcctcagcctttggttgggtctttgatatgtggtgatatcctgggtttcaactcttctacaaggtttt |
| SEQ ID | cttatatataatgtaggatagatacagttgttctgtgatttgggatggttataaaattaaataaccaaatggaaaggcagaa |
| NO: 255 | agtgagaaatgttaagtgacattgtagtaccatataatattttagatcttgacttaggcatatacaaagagtatcttactta |
| | ttttatacaaagtctacaatgttatgatgctacaaagaatgagttaagaatgagtaaacgctaggttttatggagactcata |
| | agaaaatataaagattttcgtcttactattcaagtntgaaatgcataaatgaagctttataattggtgtgcaaaaccagaca |
| | tagaacctgcccttctggactctctggtcaagtgaag |
| 242469_at | ggatattgtaaacacacctcagggagataacatggggtggaggcagacaaacaggccaatccgggggaggaatggagttaag |
| SEQ ID | gaaagctctacaatgaggcagattttttaatacccactccttccaagtccttcagctcaagctgcagccaaatatttgcata |
| NO: 256 | aagagctggtaaaggattccagcttcctggttcatgacattacagcatggatcgttttcaaaattaatactgcctcctgttt |
| | cagcagtggctgcctgcacagctctccctgtggctcagagtatgggagtgagtatgccctgtaacccaaaagaggtgtggtg |
| | agctcngcttcccagcacagaatcacagagcaatacgggtacct |
| 242765_at | caagactacactttcttgccttctttctatttattctttttgtgcgtataaatggtatgttgtgatatattctcagtgcttg |
| SEQ ID | tgcccaccttggaactctgttcttgctcttcattccgcatgtgatactctggtccaagatcttggccaggtgccttctgctc |
| NO: 257 | aaatatcgtctcagaggtgcttcccttgaaaactcggtgctgtttccatagttactctatttgatcactctaagtttggttg |
| | tcttcatagcacttgtcaccctctggaactattctattcatttatttacttgtttaatgcttggctcttttcccctcctaac |
| | gtaaactccatgattgccaacacctgtt |
| 242874_at | gcagttccagtctcacagaagtctgctggagaatttcctctggcttggagaaggtggtcttttcattctactcaggtcttct |
| SEQ ID | attgattggataangtgncacatacatcatggagggcaatcttctttactcagaggacatcagtgtcaatgttattcccatc |
| NO: 258 | cagaaacaccttccaagttgacacataaaattaacaatacacttttcctcatctcacccaaatttaggttaagagttctgga |
| | tacctgaattaatattgccattggaatgccacttttacttttggagaaaaattagaaataacattttataaaattaaaaaac |
| | tgtaggctnggcacactggtttacacctataatc |
| 243010_at | gaaatacacccactctcttggaataatgacgtaccactcagttggaccctcaagagtcactgctttgtctgtgctggtagtt |
| SEQ ID | tgtgagaagtgacccgcacgcttccatttgatgcatttgatgtgagtgaatccatacatttgaatgtcattgtccttgagac |
| NO: 259 | cctacatgtgcagtttggctcatctcattaaagatgcttgatgtaataattggttagtttccttttattttcctgcaggctt |
| | ttccatgagtattatttttttcaaagaacaaatctgtatggcttttccccatctccatattttgttttgctatgaattgctt |
| | tgctttggtgaacttgtcctagtatgcttgcctcacaaacgttttagccattgtgaattttcttcatctctgtaaatagttc |
| | atctgtgcttctccctgatgacgttttattttttttcccctgtaagcaaccgaggtagaaaaataaattgtttaccatggan |
| | anannnnntgctgccgtctcttagcctgacagtgtcctgttctc |
| 243170_at | tcatataatatgttgctgcttttgggggggtgttggggagtaccagagaggtctgcatgcatctgcagtgtacactggcaaa |
| SEQ ID | caaaaggtttgtcattcccgttgtcctgttgtgcttcctttccacctctccctggctgttcccccacccccttgttcctttt |
| NO: 260 | gtaaaggatgcaggtgtagtcttgtgccagtctgtgacataacacctgaacaatttctatggtatcctacccatttgctcag |
| | attcaaaccaggccttaatcgataacaagca |
| 244008_at | tacatttcacatttaccagcaagtcagtaaaaaatagtgcttatttacatagtcaatataatttaatgttctaaaaataata |
| SEQ ID | tcttcgatctgcccaatatttaatgtatcatttgagatttttaaaaatgcanccgctccattatgtaaacattaagatatgc |
| NO: 261 | ctatgtttctttaactatacagcctctttacaataaatttcttgatttttgtgcacaggatagtattgcaacctgctattta |
| | gcctttggtgccttagaattattataaatatttaacaatatgtacataatgtaaatactgccaagagatcagtaaggccaaa |
| | tattttctctattcactttttattgctcttgctttctattgctactaaagcctcttttatccagctttgtaatagttccaac |
| | attgtagcgaa |
| 244272_s_at | tatcatcatatatttgtcctctagcatgtataacaccaagaggggactctaatgtaaaccatgaactttggatgataatgat |
| SEQ ID | gtttcagagtaggttcattgattgtaacaaatgtaccattggatggtaatttggaaagcaaataatcctcagtacagccaag |
| NO: 262 | ctgaggctttacagaaacagaagtcaatggaggtacagagatagtagcattttgactatttgggactgctgctttaaaatct |
| | ctttccacagaaaagttgtgtttttctgtttcaccatagaaacatcctccacagcaactctttataaattctgttgccatta |
| | cattcaatttccaatatccagcaaaagacacaaacttccaatcttaatattaatct |
| 244335_at | atgccagaattaccaaaagtcacgaaacacaaacagcaaataaagtgtaatgttacagctataacaactcacataaagtatg |
| SEQ ID | cagaaacaactaggaaacactgagaattttaaatctgattaaggagctagtagagattacccagcattcagctcaccatttg |
| NO: 263 | aaaccagccaaatgaatatgtgacaagtctcttttaaaaatcttgcactttgaaaaggaaatgtgttataagtgttactcaa |
| | tctggaagttgcaatgagcttgacaatgtcaaaacatggatttagagattctcaatgaacctctaatgttttaccttcaata |
| | aaatctcttttcataaaaggaaaaaaaagattacccaaattctaccatcattttgccactgggattattttcctttcaaaca |
| | atttggatgaccattattattcctggacttgaatgtacacgattctcataaacccaat |
| 244370_at | tgcccccctaggtgctaataaaaggatatttacttcaaagtttctggtttnagaannccncaatncaaagaaaaaaaataca |
| SEQ ID | cttttgttggaaattttgtancncaaancatgttgtgaaagagtgtagtgatcttgtgcaaaaaaaatgaacatttgtgagc |
| NO: 264 | ttcttgctgttttatagattttcttgtaaattattcttgtaacncttctggttttgttgttcttgttgcgaaggtttcaaat |
| | atttgtgactgaatgtacggtgaaactgtcataatgttacctagctgagttttgttat |
| 244393_x_at | cccagccgctataacttttaacaattcccatatgtcctttattccactaagatgagtgcagtatatatttccatctgtccaa |
| SEQ ID | ggcttcctaaatgtagccaangccaagccaacaccagtcacatgatcnaaatcaaagggcatttggggaatccaggctgtga |
| NO: 265 | ttcagggaagttccaagtgtctgatgaagtgtttgttttacatctttgtgtcccttgcaggtctagcactgtgctatgtagg |
| | taacatgtgctcc |
| 35685_at | gaggagcctgctttgcccagcctggagggcgtcagtgaaaagcagtacaccatctacatcgcacctggaggcggggcgttca |
| SEQ ID | cgacnnnnnnnnnnnnnnnnnnnnnnnnnnnnnnnnnnnnnnnattctggaaggtgtcccggccactggagctgtgctatgc |
| NO: 266 | tcccaccaaggatccaaagtgaccccaccaggggacagccagaggaaggggaccatggggtatccctgtgtcctggtctatc |
| | accccagcttctttgtcccccagtacccccagcccagccagccaataagaggacacaaatgaggacacgtggcttttataca |
| | aagtatctatatgagattcttctatattgtacagagtggggcaaaacacgcccccatctgctgccttttntattgccctgca |
| | acgtcccatctatacgaggtgtt |
| 39582_at | ggccacaaatggagggattgtcctttcaagcaccacagcttcagataaaattagtactttcaaatattgtccactttaactt |
| SEQ ID | aaaaaattctagagggattatattggagactcaactgcccttnggttttagtttataaaatggcctagtactgtggaatttt |
| NO: 267 | aattttagaaagtcttagcatcagatcataaacattcattaaaagaactcacatcccatctgaaacttcccaggggagttgg |
| | gattcttagtagattggtagaaaggggctcattttctactgcatttcccatttttggtatcttgttcagcatgttttatttt |
| | tatttcttgtctgcagaacatcctatatttatgagaacattctttaagaagaccaccacatagaataccccttcctatcagc |
| | tcgctctgatttagccttaat |
| 52164_at | aagatgccagccacggactcgtgccagcccagcacccaaggccagtacatggtggtcaccactgagcccctcacccaggccg |
| SEQ ID | tggtagacaaaactctccttctggtggtgctgttactcggggtgacccttttcatcacagtcttggttttgtttgccctgca |
| NO: 268 | ggcctatgagagctacaagaagaaggactacacccaggtggactacttaatcaacgggatgtatgcggactcagaaatgtga |
| | ggggggcgggggnctggcgggaggcctggccccttcctcgtcctttccttttgcctttgagaccaaaccaagtgcttccaaa |
| | ttcttttggtgcaattgaggagatatgccagatgcttaaacacatttaattgctgtcagattaattccatgatcactaaaga |
| | gttgctgcttttttcatannnannnntgtaaatgattctgtgcccaggagcag |

TABLE 11
100 PS gene list, target sequences

| | |
|---|---|
| Probe Set ID | Target sequence |

| | |
|---|---|
| 1555852_at | ccattctgagtacttctccgcaaaccctttgtttcattaaggactgttttacatgaagggtgcaaaagtaggataaaaatga |
| SEQ ID | gaaccctagggtgaaacacgtgacagaagaataaagactattgaatagtcctcttctctacccatggacnttggnattttta |
| NO: 269 | tattngattttaaggaaatataacttagtagtaaagagatgagcattcaagtcaggcagacctgaatttgggtcaaggctgc |
| | gccactcaaaagctatatgacctctatatgagcagcttattcaacctcttttaacctccattttgtcatctgtagaatgatg |
| | ataaatgcctagctcagaaggattcc |
| 1556049_at | aaactccagggctcattagtccttttataaacgccggtgtgaaatgatgggtatccgtgtgcctggtccaagtttcctggtg |
| SEQ ID | gttgacttgggcgaataaaaataagacatgttctgcagagttctccttttaaattcttgtgtaatattccgtttgttttgct |
| NO: 270 | cctttgcaattaactacaaacttctaagtcctagaaaagatttcaagtatttatagagtgagcaacgatcattttcctagtc |
| | aaatagtatggcaaaaatcactgttttaaaaagttgtggcattaaactataaatcctccctcccctcgcttcccacccccaa |
| | ttatgaaagaaaagcatatgacaatgcctactgggctcagcttttgggctaattgagtctgactgagccttcttagcaggtt |
| | cctgtcttttgaaacctcaaatcccaaaaagctgtctaataattttacttttttagggactaagaccaggtattttcagcag |
| | aggttcttacttttctgtaattgaggcgcaac |
| 1561042_at | aaatgcagataaaggctacctctgaattctcaatagattcatcatgtttgctcttaagtgtagctgtccacactg |
| SEQ ID | |
| NO: 271 | |
| 1563497_at | taccatagcaagttcttatcctttgttaatatattataaatacgacttttttcctttacctgtcacagtaatatgccaattt |
| SEQ ID | ttggtgtcgctttagtaatttttttgggggggactgtcaaatttcagtaaactttataggctatagtttacaatttaaggtc |
| NO: 272 | tttcctcagttgtctcctaaaaaattttgtggaaatcgaaatttcttttcaattgatcgtcttgtgcagccctttaaaaaat |
| | gcgttttgcattcatgtctttgcagtacaaatctgtatctttcattttgggagtgactgtcatagatgggtaatataaagtg |
| | acaacacacacaccagggccagattgcgtgggttctagctagggacctagagctggttacttgatttctctctgttccattt |
| | actatctg |
| 200615_s_at | gggcaggacatgctgtaccaatccctgaagctcactaatggcatttggattttggccgaactacgtatccagccaggaaacc |
| SEQ ID | ccaattacacgctgtcactgaagtgtagagctcctgaagtctctcaatacatctatcaggtctacgacagcattttgaaaaa |
| NO: 273 | ctaacaagactggtccagtacccttcaaccatgctgtgatcggtgcaagtcaagaactcttaactggaagaaattgtattgc |
| | tgcgtagaatctgaacacactgaggccacctagcaaggtagtaactagtctaacctgtgctaacattagggcacaacctgtt |
| | ggatagttttagcttcctgtgaacatttgtaaccactgcttcagtcacctcccacctcttgccacctgctgctgctatctgt |
| | ccttacttgtgggcttctccatgctgtgccaatggctggctttttctacacc |
| 200904_at | tgtgccttcattcatgggttaatggattaatgggttatcacaggaatgggactggtggctttataagaagaggaaaagagaa |
| SEQ ID | ctgagctagcatgcccagcccacagagagcctccactagagtgatgctaagtggaaatgtgaggtgcagctgccacagaggg |
| NO: 274 | cccccaccangggaaatgtctagtgtctagtggatccaggccacaggagagagtgccttgtggagcgctgggagcaggacct |
| | gaccaccaccaggaccccagaactgtggagtcagtggcagcatgcagcgcccccttgggaaagctttaggcaccagcctgca |
| | acccattcgagcagccacgtaggctgcacccagcaaagccacaggcacggggctacctgangccttgggggcccaatccctg |
| | ctccagtgtgtccgtgaggcagcacacgaagtcaaaagagattattctcttcccacagataccttttctctcccatgaccct |
| | ttaacagcatctgcttcattcccctcaccttcccaggctgatctgaggtaaa |
| 200905_x_at | aaagcctgagacagctgccttgtgtgcgactgagatgcacagctgccttgtgtgcgactgagatgcaggatttcctcacgcc |
| SEQ ID | tcccctatgtgtcttaggggactctggcttctctttttgcaagggcctctgaatctgtctgtgtccctgttagcacaatgtg |
| NO: 275 | aggaggtagagaaacagtccacctctgtgtctaccatgacccccttcctcacactgacctgtgttccttccctgttctcttt |
| | tctattaaaaataagaacctgggcagagtgcggcagctcatgcctgtaatcccagcacttagggaggccgaggagggcagat |
| | cacgaggtcaggagatcgaaaccatcctggctaacacggtgaaaccccgtctctactaaaaaatacaaaaaattagctgggc |
| | gcagaggcacgggcctgtagtcccagctactcaggaggcggaggcatggagaatggcgtcaacccgggaggcggaggttgca |
| | gtgagccaggattgtgcgactgcactccagcctgggtgacagggtgaaacgccatctc |
| 201009_s_at | atactgaggtggatccctgcatcctcaacaacaatgtgcagtgagcatgtggaagaaaagaagcagctttacctacttgttt |
| SEQ ID | ctttttgtctctcttcctggacactcactttttcagagactcaacagtctctgcaatggagtgtgggtccaccttagcctct |
| NO: 276 | gacttcctaatgtaggaggtggtcagcaggcaatctcctgggccttaaaggatgcggactcatcctcagccagcgcccatgt |
| | tgtgatacaggggtgtttgttggatgggtttaaaaataactagaaaaactcaggcccatccattttctcagatctccttgaa |
| | aattgaggccttttcgatagtttcgggtcaggtaaaaatggcctcctggcgtaagctttt |
| 201010_s_at | gtgttctcctactgcaaatattttcatatgggaggatggttttctcttcatgtaagtccttggaattgattctaaggtgatg |
| SEQ ID | ttcttagcactttaattcctgtcaaattttttgttctccccttctgccatcttaaatgtaagctgaaactggtctactgtgt |
| NO: 277 | ctctagggttaagccaaaagacaaaaaaaattttactacttttgagattgccccaatgtacagaattatataattctaacgc |
| | ttaaatcatgtgaaagggttgctgctgtcagccttgcccactgtgacttcaaacccaaggaggaactcttgatcaagatgcc |
| | caaccctgtgatcagaacctccaaatactgccatgagaaactagagggcaggtgttcataaaagccctttgaacccccttcc |
| | tgccctgtgttaggagatagggatattggcccctcactgcagctgccagcacttggtcagtcactctcagccatagcacttt |
| | gttcactgtcctgtgtcagagcactgagctccacccttttctgagagttat |
| 202544_at | gtattcttactatggttgcctttccctatttgttcaatagactgataatactggtatttatagagtttgagccattacaact |
| SEQ ID | tttgtgaggatgtgtttcaaacatttctggacaaatcttattttgtatttctggaagaatgtagtaatcttctagaccgctt |
| NO: 278 | aaaaccaatgctcccaagctgaatattcttgagcaaatttgttttttattatgccatttgacatttcaaatcagtgctcata |
| | tacagtaaacttgtgatagaaattgtattttattgctttttggattataattcatataaatataattacttgaatattgttt |
| | gagatcattaacatgccagggcagttcccactgatttagatggtccaagataatctcattcaggaggcttgaaacattaatg |
| | gtttagtcttgtgaattttaacagttctctgtcatcgtttaacaaaaccaacaactgacacaactccttaagctgtggtttc |
| | agtctctgctagttcatattgcatgt |
| 202546_at | gatctggtgcggaacctgcaaagtgaggtggagggagttaagaatattatgacccagaatgtggagcggatcctggcccggg |
| SEQ ID | gggaaaacttggaacatctccgcaacaagacagaggatctggaagccacatctgagcacttcaagacgacatcgcagaaggt |
| NO: 279 | ggctcgaaaattctggtggaagaacgtgaagatgattgtccttatctgcgtgattgtttttatcatcatcctcttcattgtg |
| | ctctttgccactggtgccttctcttaaagtaa |
| 202828_s_at | gggggccacagggtgggctgtggagctctcaggaagggccctgaggaaggcacacttgctcctgttggtccctgtccttgct |
| SEQ ID | gcccaggcagcgtggaggggaagggtagggcagccagagaaaggagcagagaaggcacacaaacgaggaatgaggggcttca |
| NO: 280 | cgagaggccacagggcctggctggccacgctgtcccggcctgctcaccatctcagtgaggggcaggagctggggctcgctta |
| | ggctgggtccacgcttccctggtgccagcacccctcaagcctgtctcaccagtggcctgccctctcgctcccccacccagcc |
| | cacccattgaagtctccttgggccaccaaaggtggtggccatggtaccggggacttgggagagtgagacccagtggagggag |
| | caagaggagagggatgtcgggggggtggggcacggggtaggggaaatggggtgaacggtgctggcagttcggctagatttct |
| 204078_at | aacttaagatggacacagctgactggacccccatcctgcctcacccatgggtgctgcaccccagacccatcctgccacttct |
| SEQ ID | atgtctctggaccacaggatggtggtggcattgcaggttggcaagtgggctgatggggtccgccctcctcactgctgagctc |
| NO: 281 | ctcacctggacagtctcctggacaaggagtttccagctgctggctggagtctcaggccaaattgcagagggtcctccagggt |
| | cctgaagagcactggactaagagtctagtggttccagggc |
| 204116_at | ttctggctggaacggacgatgccccgaattcccaccctgaagaacctagaggatcttgttactgaataccacgggaactttt |
| SEQ ID | cggcctggagtggtgtgtctaagggactggctgagagtctgcagccagactacagtgaacgactctgcctcgtcagtgagat |
| NO: 282 | tcccccaaaaggaggggcccttggggaggggcctggggcctccccatgcaaccagcatagcccctactgggcccccccatgt |
| | tacaccctaaagcctgaaacctgaaccccaatcctctgacagaagaaccccagggtcctgtagccctaagtggtactaactt |
| | tccttcattcaacccacctgcgtctcatactcacctcaccccactgtggctgatttggaattttgtgcccccatgtaagcac |
| | c |
| 204233_s_at | gcaaacatccggaagtatcccaccaagaaacaacagctccattttatttccagttacttgcctgcattccaaaatgactttg |
| SEQ ID | aaaacctcagtactgaagaaaaatccattataaaagaagaaatgttgcttgaagttaataggtttgcccttgcatctcattt |
| NO: 283 | cctctggggactgtggtccattgtacaagccaagatttcatctattgaatttgggtacatggactacgcccaagcaaggttt |
| | gatgcctatttccaccagaagaggaagcttggggtgtgactgtggggaggactccatccacctcatcactggactgcatggg |
| | gaggcagcagagcggggtcccctctgtgcttcgactactgctcctgtggcaggaggctttgggtggctcactactgaacaca |
| 204266_s_at | tgtagatggcgactgtgttatagacggtgctcactcccaagggacagcaagtgagcagagatgtactgcaaagtcgccagtc |
| SEQ ID | actgcgtgcaaggtggcctctgcctggggccgtccagaagctgctcctttaccctcttggtcccatggctgaagcggagcag |
| NO: 284 | ctggattgctctggagcagccaaggccgccactgtggagacagagctctcccctcctgctgggcgtgtgtgacactgtagag |
| | tttcactgtactcgatgtgacttctcccctgcccttcctcctgatggagtgtgcagacagccatgcgtggccacgggggcag |
| | tgtgaggacctccctgtctcccgctcccctcccagggagcagctgcttgacctagctctttgggcctctcctgccctctgct |
| | ctgcctggagtgtcggatcctgtgagtaggctgggcctcccctgggcagggttctccaaggccggtttcccggcccttacca |
| | aacctgatgcccctgacatcatcattcttgtgggagacagcagcctgtatgtggtgtggggcgtggatcgagtgtagc |
| 204410_at | cctttcctactgtgataacgtcaagtaattggatattttgaatacatttctgcctgataatcatgctgagttctaataagcc |
| SEQ ID | ctacttccacctaatctgtttacagtcttttggtatgtttcagttacttagatggtctcataaggtttctgatacaatttga |
| NO: 285 | agacagaaatctgcatttagaatcagaaaacatggacatatttttcatatttatctagtcatatgtaattttatgctaacat |
| | tgatagtttataaatccttttcatcctttgtgcctcggttattaaggaaaaaaaaatgtccaacatacagtttttaaagtgt |
| | ggcagttttgagtagtaacttagaatgtataagattaagagttaaagaaaccgaacaataagtggcaaccaattatcttaac |
| | attggaaatactggggtgccattttgttttcaaaagttattcattgtaatccactgttttggctttca |
| 204806_x_at | cagatcctccaaaggcacacgttgcccaccaccccatctctgaccatgaggccaccctgaggtgctgggccctgggcttcta |
| SEQ ID | ccctgcggagatcacgctgacctggcagcgggatggggaggaacagacccaggacacagagcttgtggagaccaggcctgca |
| NO: 286 | ggggatggaaccttccagaagtgggccgctgtggtggtgccttctggagaggaacagagatacacatgccatgtgcagcacg |
| | aggggctgccccagcccctcatcctgagatgggagcagtctccccagcccaccatccccatcgtgggcatcgttgctggcct |
| | tgttgtccttggagctgtggtcactggagctgtggtcgctgctgtgatgtggaggaagaagagctcagatagaaacagaggg |
| | agctactctcaggctgcagtcactgacagtgcccagggctctggggtgtctctcacagctaataaagtgtgagacagcttcc |
| | ttgtgtgggac |
| 204897_at | agcagcttattgtttctctgaaagtgtgtgtagttttactttcctaaggaattaccaagaatatcctttaaaatttaaaagg |
| SEQ ID | atggcaagttgcatcagaaagctttattttgagatgtaaaaagattcccaaacgtggttacattagccattcatgtatgtca |
| NO: 287 | gaagtgcagaattggggcacttaatggtcaccttgtaacagttttgtgtaactcccagtgatgctgtacacatatttgaagg |
| | gtctttctcaaagaaatattaagcatgttttgttgctcagtgtttttgtgaattgcttggttgtaattaaattctgagcctg |
| | atattgatatg |
| 205027_s_at | attatataactctttgagcctttattggtaaattctggtatacattgaattcattataatttgggtgactagaacaacttga |
| SEQ ID | agattgtagcaataagctggactagtgtcctaaaaatggctaactgatgaattagaagccatctgacagacggccactagtg |
| NO: 288 | acagtttcttttgtgttcctatggaaacattttatactgtacatgctatgctgaagacattcaaaacgtgatgttttgaatg |
| | tggataaaactgtgtaaaccacataattttgtacatccaaggatgaggtgtgacctttaagaaaaatgaaaacttttgtaaa |
| | ttattgatgattttgtaattcttatgactaaattttcttttaagcatttgtatattaaaatagcatactgtgtatgttttat |
| | atcaaatgccttcatgaatctttcatacatatatatatttgtaacatgtaaagtatgtgagtagtcttatgtaaagtatgtt |
| | tttacattatgcaaataaaacccaatacttttgtccaatgtggttggtcaa |
| 205204_at | agatccgagtgcactcgcgaggcaacctctgggccaccggtcacttcatgggcaagaagagtctggagccttccagcccatc |
| SEQ ID | ccattggggacagctccccacacctcccctgagggaccagcgactgcagctgagtcatgatctgctcggaatcctcctgcta |
| NO: 289 | aagaaggctctgggcgtgagcctcagccgccccgcaccccaaatccagtacaggaggctgctggtacaaatactgcagaaat |
| | gacaccaataataggggcagacacaacagcgtggcttagattgtgcccacccagggaaggtgctgaatgggaccctgttgat |
| | ggccccatctggatgtaaatcctgagctcaaatctctgttactccattactgtga |
| 205273_s_at | ggctgtcgactgggctaagtctggaaaattcacacagcaagacatcgacgaagccaaactttctgtcttctcaaccgtagat |
| SEQ ID | gctcctgtcgctccttcagacaaaggaatggaccacttcttgtacggcctctcggatgagatgaagcaggcccacagagagc |
| NO: 290 | agctctttgctgtcagccacgacaagctcctggccgtgagcgatagatacctcggcactgggaagagcacacacggcctggc |
| | catcctcggacccgagaacccgaaaattgccaaggacccatcctggatcatccgatgagcagccgtggcgctcgactgcaca |
| | ggagcccgagacaatacacctccgagctgaatatgaaaagtcagaaatgctactgctttttccaagaatattatgtcattga |
| | gtgtcgccaaagcccttgactggcgagtcaaaaactcagatctatcttaagagtgaccaggaagaggttcattgaaataatc |
| | atgcatgaagcgccaaagatgcaccatgtagaattttcactttgtactggcaggctcg |
| 205419_at | cagcccctgaagaaaattcacgtgaaatgacagaaacgcagatgatgatacattccaagtcttcaaatggaaagtgaaatgg |
| SEQ ID | attgtattttggtttatagtgacgtaaactgtatgacaaactttgcaggacttcccttataaagcaaaataattgttcagct |
| NO: 291 | tccaattagtattcttttatatttctttcattgggcgctttcccatctccaactcggaagtaagcccaagagaacaacataa |
| | agcaaacaacataaagcacaataaaaatgcaaataaatattttcatttttatttgtaaacgaatacaccaaaaggaggcgct |
| | cttaataactcccaatgtaaaaagttttgttttaataaaaaattaattattattcttgccaacaaatggctagaaaggactg |
| | aatagattatatattgccagatgttaatactgtaacatactttttaaataacatatttcttaaatccaaatttctctcaatg |
| | ttagatttaattccctcaataacaccaa |
| 205696_s_at | ttcctgttctcttgtatagctgaaattccagtttaggagctcagttgagaaacagttccattcaactggaacattttttttt |
| SEQ ID | ttccttttaagaaagcttcttgtgatccttcggggcttctgtgaaaaacctgatgcagtgctccatccaaactcagaaggct |
| NO: 292 | ttgggatatgctgtattttaaagggacagtttgtaacttgggctgtaaagcaaactggggctgtgttttcgatgatgatgat |
| | catcatgatcatgatgattttaacagttttacttctggcctttcctagctagagaaggagttaatatttctaaggtaactcc |
| | catatctcctttaatgacattgatttctaatgatataaatttcagcctacattgatgccaagcttttttgccacaaagaaga |
| | ttcttaccaagagtgggctttgtggaaacagctggtact |
| 205890_s_at | gatcttaaagccacggagaagcctctcatcttatggcattgacaaagagaagaccatccaccttaccctgaaagtggtgaag |
| SEQ ID | cccagtgatgaggagctgcccttgtttcttgtggagtcaggtgatgaggcaaagaggcacctcctccaggtgcgaaggtcca |
| NO: 293 | gctcagtggcacaagtgaaagcaatgatcgagactaagacgggtataatccctgagacccagattgtgacttgcaatggaaa |
| | gagactggaagatgggaagatgatggcagattacggcatcagaaagggcaacttactcttcctggcatcttattgtattgga |
| | gggtgaccaccctggggatggggtgttggcaggggtcaaaaagcttatttcttttaatctcttactcaacgaacacatcttc |
| | tgatgatttcccaaaattaatgagaatgagatgagtagagtaagatttgggtgggatgggtaggatgaagtatattgcccaa |
| | ctctatgtttctttga |
| 206035_at | aatggattgattcctgcctttattcttgtaataatagcttcttttacttcttttttcttcacacatcgaatacccaaatttt |
| SEQ ID | ggaaaaacaaaggtctgcgttcttgtccaaattctgcttcatagtagccgtctctgcagtcttttccaactaaatcatgagg |
| NO: 294 | atgaggtttatatgggtcattctttgttactaatgtaattctcacttttccttttccataatagttcataatctggatagaa |
| | gggtatgttcggttgttgtctgtgctgtgctcccctggaatgctgcctgctgatcgcccttcacatttgtatctaaaacgca |
| | ttcccctctgcctgggttgttcaattatctctatatacgggttatacgcaccggaggc |
| 207397_s_at | gatgtggctctaaatcagccggacatgtgcgtctaccgaagagggaggaagaagagagtgccttacaccaaactgcagctta |
| SEQ ID | aagaactggagaacgagtatgccattaacaaattcattaacaaggacaagcggcggcgtatctcggctgctacgaacctatc |
| NO: 295 | tgagagacaagtgaccatttggtttcagaaccgaagagtgaaggacaagaaaattgtctccaagctcaaagatactgtct |
| 207651_at | ttgccttgtaattcgacagctctacagaaacaaagataatgaaaattacccaaatgtgaaaaaggctctcatcaacatactt |
| SEQ ID | ttagtgaccacgggctacatcatatgctttgttccttaccacattgtccgaatcccgtataccctcagccagacagaagtca |
| NO: 296 | taactgattgctcaaccaggatttcactcttcaaagccaaagaggctacactgctcctggctgtgtcgaacctgtgctttga |
| | tcctatcctgtactatcacctctcaaaagcattccgctcaaaggtcactgagacttttgcctcacctaaagagaccaaggct |
| | cagaaagaaaaattaagatgtgaaaataatgcataaaagacaggattttttgtgctaccaattctggccttactgga |
| 208296_x_at | ttgagttctccttttaagtaccaatgatacttaaatttctcagaaatgtaatggtgtgtcattgccttgaaatgcttgctta |
| SEQ ID | gggcttcttttatgttatcttaaaaagtgctggtgaattttccattttttacatccatttcacatgtaagagacacaaaagt |
| NO: 297 | ctagattggtcttgatattgagataataaaaagtaagtagcattaagaaaggtaacaatcttcattctacagatgaactcat |
| | tgaaacaatttaggggaatgaggggcaaaaggggagaaatactgctaaagaacatgagcataaaaatgcgtgcgtttcagtg |
| | tttaagaaggcttgataaagaatgtcacttttttatttaactgataagatttttgttattttttactttgataagtaaacca |
| | aagaatatttgtatttcaagcagtttgtgtggtgtttctatataattttctgtgtataaataataaagtaggcatttgttta |
| | ttttgtaaaaaagaaatgaaaatctgctggccagctatgtcctctaggaaatgacagacccaaccacca |
| 208478_s_at | agggtttcatccaggatcgagcagggcgaatggggggggaggcacccgagctggccctggacccggtgcctcaggatgcgtc |
| SEQ ID | caccaagaagctgagcgagtgtctcaagcgcatcggggacgaactggacagtaacatggagctgcagaggatg |
| NO: 298 | |
| 208799_at | ctggcctctactacgtggacagtgaagggaaccggatttcaggggccaccttctctgtaggttctggctctgtgtatgcata |
| SEQ ID | tggggtcatggatcggggctattcctatgacctggaagtggagcaggcctatgatctggcccgtcgagccatctaccaagcc |
| NO: 299 | acctacagagatgcctactcaggaggtgcagtcaacctctaccacgtgcgggaggatggctggatccgagtctccagtgaca |
| | atgtggctgatctacatgagaagtatagtggctctaccccctgaaagagggtggatgcagctgcttgtgtttcttggggtga |
| | ctgtcattggtaatacggacacagtgacccatcctccatcctatttatagtggaagggccttcaattgtatcagtacttttt |
| | tttaagctctggcacattgacctctat |
| 208885_at | gaagtaagcctcatcatcagagcctttcctcaaaactggagtcccaaatgtcatcaggttttgttttttttcagccactaag |
| SEQ ID | aacccctctgcttttaactctagaatttgggcttggaccagatctaacatcttgaatactctgccctctagagccttcagcc |
| NO: 300 | ttaatggaaggttggatccaaggaggtgtaatggaatcggaatcaagccactcggcaggcatggagctataactaagcatcc |
| | ttagggttctgcctctccaggcattagccctcacattagatctagttactgtggtatggctaatacctgtcaacatttggag |
| | gcaatcctaccttgcttttgcttctagagcttagcatatctgattgttgtcaggccatattatcaatgtttacttttttggt |
| | actataaaagctttctgccacccctaaactccaggggggacaatatgtgccaatcaatagcacccctactcacatacacaca |
| | cacctagccagctgtcaagggc |
| 209140_x_at | gacctggcagcgggatggcgaggaccaaactcaggacaccgagcttgtggagaccagaccagcaggagacagaaccttccag |
| SEQ ID | aagtgggcagctgtggtggtgccttctggagaagagcagagatacacatgccatgtacagcatgaggggctgccgaagcccc |
| NO: 301 | tcaccctgagatgggagccatcttcccagtccaccgtccccatcgtgggcattgttgctggcctggctgtcctagcagttgt |
| | ggtcatcggagctgtggtcgctgctgtgatgtgtaggaggaagagttcaggtggaaaaggagggagctactctcaggctgcg |
| | tccagcgacagtgcccagggctctgatgtgtctctcacagcttgaaaagcctgagacagctgtcttgtgagggactgagatg |
| | caggatttcttcacgcctcccctttgtgacttcaagagcctctggcatctctttctgcaaaggcacctgaatgtgtctgcgt |
| | ccctgttagcataatgt |
| 209567_at | ccgcctggcttgggtggcaagagaaaaggaggacagcgcccaggaggaaagaggaggaagtaatagtttctaactgtcggac |
| SEQ ID | ccgtctgtaaaccaaggactatgaatactaaatgttaagttctaggcaattatacggggactcagaaggacctggccgctgc |
| NO: 302 | cttcattgagtttaaagggacaggattgcccttccgtcaagaaagtatgtaagtgttggactgcacaaattaatgtttttcc |
| | cacaaccgagactttggagattaagaacttatttgaggatttaagaattagggaaataatttggtggaaaccgggaatgagt |
| | tctattcttaaacagcctttttttttctttttaatgttggatatacggcgaggtagagttggccatatttcagagacttaga |
| | ttgacgtatatgtttctgcattatttttacaacaagtttgtgtatcagagcgggagtgcgggggag |
| 209606_at | gaattgcaaaactgacatcccatttcacagcaatagtgacctttatttaaattgttgtgttatagtttatgcttcttaaatc |
| SEQ ID | atttttcaacctaaacagccaatttctaagcagacaggaaaactaaataataagttaattaatataacaaagatgcaggttc |
| NO: 303 | ctgctcattccagtaatgtctttgaaagcaaaactaatatttattttctagattatccctgtgaataattgagaactttttg |
| | gagtcaagtatgaataaaggtgtggcagaatataataatctggactattttctataggataattgctgggttataaaatctt |
| | aggtttgcttatgcccagtagctcctgcggaggcttaataataggcaattttgaatttgttcaaacctgtaatggcttgtaa |
| | acaaagatgaccatcagctgtttctcacatctatagtgacaataaagcgggaagtataagatttaataggaggggttaaggt |
| | tcatgagaaccatggaaagatgtggtctgagatgggtgctgcaaagat |
| 209774_x_at | agagagacacagctgcagaggccacctggattgcgcctaatgtgtttgagcatcacttaggagaagtcttctatttatttat |
| SEQ ID | ttatttatttatttatttgtttgttttagaagattctatgttaatattttatgtgtaaaataaggttatgattgaatctact |
| NO: 304 | tgcacactctcccattatatttattgtttattttaggtcaaacccaagttagttcaatcctgattcatatttaatttgaaga |
| | tagaaggtttgcagatattctctagtcatttgttaatatttcttcgtgatgacatatcacatgtcagccactgtgatagagg |
| | ctgaggaatccaagaaaatggccagtaagatcaatgtgacggcagggaaatgtatgtgtgtctattttgtaactgtaaagat |
| | gaatgtcagttgttatttattgaaatgatttcacagtgtgtggtcaacatttctcatgttgaagctttaagaactaaaatgt |
| | tctaaatatcccttggacattttatgtctttcttgtaagatactgccttgtttaatgttaattatgcagtgtttccctc |
| 209849_s_at | tactggcacaagtttatcttcttccagatttcctttcagaacactcaaaggttcgactagtgatagtggatggtattgcttt |
| SEQ ID | tccatttcgtcatgacctagatgacctgtctcttcgtactcggttattaaatggcctagcccagcaaatgatcagccttgca |
| NO: 305 | aataatcacagattagctgtaattttaaccaatcagatgacaacaaagattgatagaaatcaggccttgcttgttcctgcat |
| | taggggaaagttggggacatgctgctacaatacggctaatctttcattgggaccgaaagcaaaggttggcaacattgtacaa |
| | gtcacccagccagaaggaatgcacagtactgtttcaaatcaaacctcagggatttagagatactgttgttacttctgcatgt |
| | tcattgcaaacagaaggttccttgagcacccggaaacggtcacgagac |
| 209894_at | ggcataggaacagttttctcttcatatattatgattatgaataataggaaagttgtattaattcagtatttgtcattatgca |
| SEQ ID | gtattttaatacctacataagtctattccattattatattttgcgcttggcatatttattcctttatgcttttaatcactaa |
| NO: 306 | catattttacttaagagtataaaactatgctaaataaattgtattgtatatggaatatgcttgccattatgaagaacagctg |
| | ggtataccattatttcaagtaataaatctgaattctattagtttaaaaattgtaaaattcaagttaaatgacgtgtatgata |
| | tataagctgaacacattttctatagcccttaatttagttgtgttaatttttcaaggtgatgtatcaacagcttttttttttt |
| | gcatttgttttttcaatgtgtttacattgtatgaattgagctttttgcccacagattcttgatttgtagttgtttggcagga |
| 210260_s_at | gattgagtcatcgacattcaggatttaagtctgaggtagtcaaccctcaggaaaaaaaaaatggcttatctgaaatcagtac |
| SEQ ID | tgtggaaatgaactatattagctattatgaataatgtccagtataagaatatgcttctggaattgagttctccttttaagta |
| NO: 307 | ccaatgatacttaaatttctcagaaatgtaatggtgtgtcattgccttgaaatgcttgcttagggcttcttttatgttatct |
| | taaaaagtgctggtgaattttccattttttacatccatttcacatgtaagagacaaaaaagtctagattggtcttgatattg |
| | agataataaaaagtaagtagcattaagaaaggtaacaatcttcattctacagatgaactcattgaaacaatttaggggaatg |
| | aggggcaaaaggggagaaatactgctaaagaacatgagcataaaaacgcgtgcgtttcagtgtttaagaagg |
| 210538_s_at | gaaagtgtcctatttgtaggagtacaatcaagggtacagttcgtacatttctttcatgaagaagaaccaaaacatcatctaa |
| SEQ ID | actttagaattaatttattaaatgtattataactttaacttttatcctaatttggtttccttaaaatttttatttatttaca |
| NO: 308 | actcaaaaaacattgttttgtgtaacatatttatatatgtatctaaaccatatgaacatatattttttagaaactaagagaa |
| | tgataggcttttgttcttatgaacgaaaaagaggtagcactacaaacacaatattcaatcaaaatttcagcattattgaaat |
| | tgtaagtgaagtaaaacttaagatatttgagttaacctttaagaattttaaatattttggcattgtactaatacctggtttt |
| | ttttttgttttgtttttttgtacagacagggcagcatactgagaccctgcctttaaaaacaaacagaacaaaaacaaaacac |
| | cagggacacatttctctgtcttttttgatcagtgtcctatacatcgaaggtgtgcata |
| 211529_x_at | caccaccctgtctttgactatgaggccaccctgaggtgctgggccctgggcttctaccctgcggagatcatactgacctggc |
| SEQ ID | agcgggatggggaggaccagacccaggacgtggagctcgtggagaccaggcctgcaggggatggaaccttccagaagtgggc |
| NO: 309 | agctgtggtggtgccttctggagaggagcagagatacacgtgccatgtgcagcatgaggggctgccggagcccctcatgctg |
| | agatggaagcagtcttccctgcccaccatccccatcatgggtatcgttgctggcctggttgtccttgcagctgtagtcactg |
| | gagctgcggtcgctgctgtgctgtggagaaagaagagctcagattgaaaaggagggagctactctcaggctgcaagcagtga |
| | cagtgcccagggctctaatgtgtctctcacggcttgtaaattgtgaaacagctgccctgtgtgggactgagt |
| 211595_s_at | acacagatccaggtagtctctgctagtaatgagccccttgcctttgcttcctgtggcacagagggatttcggaatgccaaga |
| SEQ ID | agggcacaggcatcgcagcacagacagcaggcatagccgcagcggcgagagctaaacaaaagggcgtgatccacatccgagt |
| NO: 310 | tgtggtgaaaggcctggggccaggacgcttgtctgccatgcacggactgatcatgggcggcctggaagtgatctcaatcaca |
| | gacaacaccccaatcccacacaacggctgccgccccaggaaggctcggaagctgtgatgggaaggaggcctgcacttggacc |
| | tgacctcaagcctcagctccagtgggaccttgtaaaatgctccctgtcagagctctccagaatatgcttgttggagatcctt |
| | caggcagtaagggagagttttgcctccttacacagtggcctttgcttgcacctccagctggagatgggtgtgccccagaagt |
| | aagctttgc |
| 211603_s_at | ggtgctgccctgtgtacatataaatgaatctggtgttggggaaaccttcatctgaaacccacagatgtctctggggcagatc |
| SEQ ID | cccactgtcctaccagttgccctagcccagactctgagctgctcaccggagtcattgggaaggaaaagtggagaaatggcaa |
| NO: 311 | gtctagagtctcagaaactcccctgggggtttcacctgggccctggaggaattcagctcagcttcttcctaggtccaagccc |
| | cccacaccttttccccaaccacagagaacaagagtttgttctgttctgggggacagagaaggcgcttcccaacttcatactg |
| | gcaggagggtgaggaggttcactgagctccccagatctcccactgcggggagacagaagcctg |
| 211700_s_at | ccagcacgggcttcagtagtggacccagttctattgttggcttcagcggtggaccaagcactggtgttggcttctgcagtgg |
| SEQ ID | accaagcaccagtggcttcagcggtggaccgagcacaggagctggcttcggcggtggaccaaacactggtgctggctttggt |
| NO: 312 | ggtggaccgagcaccagtgctggctttggcagtggagccgccagtcttggtgcctgtggcttctcgtatggctagtgagg |
| 212349_at | tggagtgtgttcctgaagagcagccaggaggccagcatggctggagaggcaggcataggcagggaaccgagcagcaggtcag |
| SEQ ID | agcaggcgagctgacattctgcagcctggacggccatggcaggaagcttttagttggagagatacaggaagcctcctagggt |
| NO: 313 | tctgagcagaagaggggcatgagctgattcacattctgaaggacctctctagctggccagtgctgaggaggttggagagaga |
| | aagggtgaaagcagagagaccagtgcagggctgttaacagggttgcaggcgagagactggggtgctgggctcccctagacta |
| | ggactccagtgccctcctctcccaagagacaaaggccattgc |
| 212654_at | ggtggccgagagtaaatgtggggacctagaggaggagctgaaaattgttaccaacaacttgaaatccctggaggcccaggcg |
| SEQ ID | gacaagtattccaccaaagaagataaatatgaagaggagatcaaactgttggaggagaagctgaaggaggctgagacccgag |
| NO: 314 | cagagtttgccgagaggtctgtggcaaagttggagaaaaccatcgatgacctagaagatgaagtctatgcccagaagatgaa |
| | gtacaaggccattagcgaggaactggacaacgcactcaatgacatcacctccctctgagccccacgccagcgtggccacctc |
| | agctctcttctctcctctcctttccattctctctatggggaggggagcaggcaggaggagcagaaattgccaacattgcaca |
| | gccaggctgggagcagcctagggagagcccc |
| 212671_s_at | accaatgaggttcctgaggtcacagtgttttccaagtctcccgtgacactgggtcagcccaacaccctcatctgtcttgtgg |
| SEQ ID | acaacatctttcctcctgtggtcaacatcacatggctgagcaatgggcactcagtcacagaaggtgtttctgagaccagctt |
| NO: 315 | cctctccaagagtgatcattccttcttcaagatcagttacctcaccttcctcccttctgatgatgagatttatgactgcaag |
| | gtggagcactggggcctggatgagcctcttctgaaacactgggagcctg |
| 212877_at | gaggaaaatgaagctcgggctggttaactgacttgctcagcgtcccatggcctagccgcccgtgactctcacactgtctcct |
| SEQ ID | gcatgacgggtggcgcctcccgcagcttcccttctctctccagtgctgcccgctgtgtctagcagcctctaggatcttgtca |
| NO: 316 | gagctgcacctctctgtgaactggccattcctttcggtgctgctgtcctttttgggggggttcctgatttctgtatacatgt |
| | agctttgccagatatgtacttagtaatataaactgtattaataaaatccatttactgtgt |
| 213008_at | tacatttttccacgagctggtgcagacagctctgccatcaggcagctgtgtggacaccttgttaaaggacttgtgcaaaatg |
| SEQ ID | tacaccacacttacagcccttgtcagatattatctccaggtgtgtcagagctccggaggaattccaaaaaatatggaaaagc |
| NO: 317 | tggtgaagctgtctggttctcatctgacccccctgtgttattctttcatttcttacgtacagaataagagtaagagcctgaa |
| | ctatacgggagagaaaaaggagaaacctgctgccgttgccacagccatggccagagttcttcgggaaaccaagccaatccct |
| | aacctcatctttgccatagaacagtatgaaaaatttctcatccacctttctaagaagtccaaggtgaacctgatgcagcaca |
| | tgaagctcagcacctcacgagacttcaagatcaaaggaaacatcctagacatggttcttcgagaggatggngaagatgaaaa |
| | tgaagagggcactgcatcagagcatgggg |
| 213539_at | gggaacactgctctcagacattacaagactggacctgggaaaacgcatcctggacccacgaggaatatataggtgtaatggg |
| SEQ ID | acagatatatacaaggacaaagaatctaccgtgcaagttcattatcgaatgtgccagagctgtgtggagctggatccagcca |
| NO: 318 | ccgtggctggcatcattgtcactgatgtcattgccactctgctccttgctttgggagtcttctgctttgctggacatgagac |
| | tggaaggctgtctggggctgccgacacacaagctctgttgaggaatgaccaggtctatcagcccctccgagatcgagatgat |
| | gctcagtacagccaccttggaggaaactgggctcggaacaagtgaacctgagactggtggcttctagaagcagccattacca |
| | actgtacct |
| 214901_at | cgaagccggcggcgtgaacaatcctcgagcaggaactcacacctggttcagcatcaacacccgaactccagaaagagctctg |
| SEQ ID | caggcggagcaaaggcagggcagccggaaagcagagccctggctttgtttgacatccaaaaaatcatgcaagagaaaaaccc |
| NO: 319 | tgtgcacgttattggggtggaagagccttctgtgggtgcttccatgttatttgacatcagagaatccacataggagagaaac |
| | tttgctgatgacttttaaccacaagtaaaaaatgtggtaagtccacatagtgtactcatggaaggaggggctgggggtagaa |
| | atgtcatgggtgacttctgactttctaaggaaatgatgcttcccaagcacccgaggttggttggtcccaaatctatcaaact |
| | cagtgccctctttagcgacatattttgtgacattccttccattacaccacagtgagttcacaggtaatataacctacccacc |
| | tgtgtaatgtcaaaaaaaatcaatatgcggccccattttgta |
| 216438_s_at | acagagacgcaagagaaaaatccactgccttccaaagaaacgattgaacaggagaagcaagcaggcgaatcgtaatgaggcg |
| SEQ ID | tgtgccgtcaatatgcactgtacattccacaagcattgccttcttattttacttcttttagctgtttaactctgtaagatgc |
| NO: 320 | aaagaggttggatcaagtttaaatgactgtgctgcccctttcacatcaaagaactactgacaacgaaggccgcgactgcctc |
| | tcccatctgtctatctggctggcagggaaggaaagaacttgcatgttggtgaaggaagaagtggggtgggacaacagtgaaa |
| | tctaaagtaaaaccaagctggcccaaggtgtcctgcaggctgtaatgcagtttaatcagagtg |
| 216920_s_at | cactactgctgcagctcacaaacacctctgcatattacatgtacctcctcctgctcctcaagagtgtggtctattttgccat |
| SEQ ID | catcacctgctgtctgcttngaagaacggctttctgctgcaatggagagaaatcataacagacggtggcacaaggaggccat |
| NO: 321 | cttttcctcatcggttattgtccctagaagcgtcnncnnannnnnnnnttgggctttctttctgggtttgggccatttcagt |
| | tctcatgtgtgtactattctatctattgtataatggttttcaaaccagtgggcacacagagaacctcactctgtaataacaa |
| | tgaggaatagccatggcgatctccagcaccaatctctccatgttttccacagctcctccagccaacccaaatagcgcctgct |
| | atagtgtagacagcctgcggcttctagccttgtccctctcttagtgttctttaatcagataactgcctggaagcctttcatt |
| | ttacacgccctgaagcagtcttctttgctagttgaattatgtggtgtgtttttccgtaata |
| 217456_x_at | cgacagtgcccaggggtctgagtctcacagcttgtaaagcctgagacagctgccttgtgtgcgactgagatgcacagctgcc |
| SEQ ID | ttgtgtgcgactgagatgcaggatttcctcacgcctcccctatgtgtcttaggggactctggcttctctttttgcaagggcc |
| NO: 322 | tctgaatctgtctgtgtccctgttagcacaatgtgaggaggtcagagaaacagtccacctctgtgtctaccatgaccccctt |
| | cctcacatgacctgtgttccttccctgttctcttttctattaaaaataagaacctgggcagagtgcggcagtcatgcctgta |
| | atcccagcattagggaggccgaggagggcagatcagaggtcaggagatcgaaaccatcctgctaagaggtgaaccgtctcta |
| | ctaaaaatacaaaaattagctggcgcagaggcaggctgtgatccagctactcaggagcgagc |
| 217885_at | gatgacaattcagaccaggctcaccggtgccgtcacttaggaatgctggaacaaaggacatttctcaaagttcccctgaaga |
| SEQ ID | catgccatctctagaaccttttttctccccgactctacccccacctctgttcctagagccctctgctggcgagtccagaaac |
| NO: 323 | attattgcccagaaggattatgtgtttatggattattttgccccgcctcaggagcgcaggaagtcactaccatttatattct |
| | aaaacagaactatctatgttcataggacttctgatgtgttcagataggaatcctcatgagagatcattatgctttgtgccct |
| | ggaccactgccgctctgggttctcaggaggaacaggcaagggcagcttcattctaagcctttccagtgacctcagccttgct |
| | tctcttctacaacactaaggctcctctgtcagaggaggtcgtcttgtttttgcttcattgcatgacataacccttccc |
| 217995_at | tacgtcaaagaccgctgctgcagtagctgcccagtcaggaatacttgataggacaatttctgtaattatgaagaatcaaaca |
| SEQ ID | ccaacaaagaagtatgatggctacacatcatgtccactggtgaccggctacaaccgtgtgattcttgctgagtttgactaca |
| NO: 324 | aagcagagccgctagaaaccttcccctttgatcaaagcaaagagcgcctttccatgtatctcatgaaagctgacctgatgcc |
| | tttcctgtattggaatatgatgctaaggggttactggggaggaccagcgtttctgcgcaagttgtttcatctaggtatgagt |
| | taaggatggctcagcacttgctcatcttggatggcttctgggccaaaactgcagtcactgaatgaccaagagcagcacgaag |
| | gacttggaacctatccttgtaaagagttccttgatgggtaatggtgaccaaatgcctcccttttcagtacctttgaacagca |
| | accatgtgggctactcatgatgggcttgat |
| 218363_at | tctggacatgagcctttgacctgggtggggcagaaagaaccacaaactccatctcccaatagaactttgaaattcactcagc |
| SEQ ID | ttttcctttcatgctgtttgttgcctgcttgttgcactcctcctgccccagaactgcaagatttttagcttcacccctttct |
| NO: 325 | gagagtaatgttatcttttatcagaatcagtatcagttcccctgtattctgtgcttcatcgaatttgcaagactgacctctt |
| | ttaagcatttaattcactcccagagtcatctggtcaggttgcaatatgaggacttctctgtctcctctgaagcctgggacac |
| | tgagcttacttaatacat |
| 218807_at | tgccgtcagccgaactttgttatggagggagcagcctcacacaagcagaaacactcctgtggatggtattgtagcatgtatt |
| SEQ ID | gtttattttagtcaatagaccctctccttataaatggtgtttagtcttcctgttgcatttcatgggcctgggggtttcctng |
| NO: 326 | cagaggatattggagcccctttttgtgacattaccaattacatctttgtccacgtttaatactttgttttggaaaatttaaa |
| | tgctgcagatttgtgtagagttctaataccaaagacagaagtaaatgttttccatatactttgtcttgcctgtatgcagccc |
| | ttgtgtaatatggt |
| 221265_s_at | tcacctctagaatagccacccaaagaccttcctgaggctgcctcagaagcaccacttgctgttttgaatgactctactagta |
| SEQ ID | tgagaaggatgtgaaggtggttggctggttgggctttaacttcctgggattcataatttttaagcttggaagatagctgctg |
| NO: 327 | ttcccatgatgggcacatttcctgagaagcttgaatgactgatgagcatagagcacccctgccttcctcaggaaacctgacc |
| | ggcaggggctctctggcttcctgaaagcttcacctcttccctcgtttatatctcaactgtaagggcattttcaagcttctgt |
| | tcatggaatgagcaactcagactgtctggagcttgctgagtacaaacacaccaccactaagtttcagaactttccttaggac |
| | ttg |
| 221875_x_at | tctaccctgcggagatcacgctgacctggcagcgggatggggaggaacagacccaggacacagagcttgtggagaccaggcc |
| SEQ ID | tgcaggggatggaaccttccagaagtgggccgctgtggtggtgcctnctggagaggaacagagatacacatgccatgtgcag |
| NO: 328 | cacgaggggctgccccagcccctcatcctgagatgggagcagtctccccagcccaccatccccatcgtgggcatcgttgctg |
| | gccttgttgtccttggagctgtggtcactggagctgtggtcgctgctgtgatgtggaggaagaagagctcagatagaaacag |
| | agggagctactctcaggctgcagtgtgagacagcttccttgtgtgggactgagaagcaagatatcaatgtagcagaattgca |
| | cttgtgcctcacgaacata |
| 223168_at | atacttgagattttcctccatttttatttcttcgtgaacatagagtttggggccgaaaatgtttttaaagtatgtgtttgag |
| SEQ ID | ttaaatataaagttggttcacttcaaagctaaaaaattgttaaacttgcagcttggtattgcagagaagattttataagaat |
| NO: 329 | tttgctttagagaatgccactttggctgaactacaagtgtaggccaccattataatttataaatacagcatacttcaaaact |
| | gtttgttatctcttgttaccatgtatgtataaatggaccttttataaccttgttctctgcttgacagactcaagagaaacta |
| | cccaggtattacacaagccaaaatgggagcaaggccttctctccagactatcgtaacctggtgccttaccaagttgtgcttt |
| | tctgt |
| 223593_at | ggcagctgcagacaagtggttaactggtttggcagaatggcatgttcctgctgctggaatgtttttatggattaaagttaaa |
| SEQ ID | ggcattaatgatgtaaaagaactgattgaagaaaaggccgttaagatgggggtattaatgctccctggaaatgctttctacg |
| NO: 330 | tcgatagctcagctcctagcccttacttgagagcatccttctcttcagcttctccagaacagatggatgtggccttccaggt |
| | attagcacaacttataaaagaatctttatgaagaaattaaactaggttgggcatggtgcgtcacacctataatcccagcact |
| | ttgggaggcagaggagggaggatcacttgaacccaggaattcaggctgcagtaagctacgatcacaccactgcactctggcc |
| | tgcatgcactctggcctgcatggcagaacaagaccctgtctctaaaaaaagagaaagaaatcaaactaatcatgctgctcat |
| 223646_s_at | aaaatactggacccacttctttcagaagagatgaagataccttatatgccctaaagttaataccagcagtcatattttatca |
| SEQ ID | gatgtaaatctggatgtaagctcttaatgttatactaaggcagtttcttaggctgtgacacttctttgtggtacttgttttg |
| NO: 331 | tgtgaaaggtaaattttggggagaaaacaatgtgaaaaacagaacttgttctgttgtttttggcatactgtttatgttagat |
| | acactgtgttacaatacaatattacgaagatctgcattgtattttggaatttggtttcctttcagaattattgctctggcta |
| | gcattggaaacaacaacaacaacaaaacccaaaggaaccctttgcagaagattcccttgtaaatggccctgtggcatgccca |
| | gtatctgcaatgttctagaatagaagttggcaaacctctctgtttgccaagcctgcagagttgaacatgtccataaatgtat |
| | acaatctgaccctgttttttggccctgtttctggacactgtagctga |
| 223746_at | gattcctggtctttttaacacgaagaatatctattgttttctcttttgtaggatctgtatgattttatctacttaacagata |
| SEQ ID | gcactaattagattaaaattctataagaaactttttaatttgctgttcataatttctgattggtatgcaataactgtttcaa |
| NO: 332 | tgaaaatcaatgtaatttagtattttaatatttgcacctttgtgaaatatagtaaataaattaagcactatcaccaccttca |
| | cagctacttaggagatccacaatcctgggttgggagccagtggatttcctgaaacacagatttgttaatgcgtttataaaat |
| | actgatgaaatgatattagtaatgtgcagtgaccatcattgactttcatgatacaattaagtttacataatttttatacttt |
| | tatatatcattttaattcaatcctgtgtatttattccttcttctgtaatgacca |
| 223819_x_at | gaccagctccaggagctctgcatcccccaagacctggtcggggacttggccagcgtggtatttgggagccagcggcccctcc |
| SEQ ID | ttgattctgtggcccagcagcagggggcctggctgccgcatgttgctgactttcggtggcgggtggatgtagcaatctccac |
| NO: 333 | cagtgccctggctcgctccctgcagccgagcgtcctgatgcagctgaagctttcagatgggtcagcataccgctttgaggtc |
| | cccacagccaagttccaggagctgcggtacagcgtggccctggtcctaaaggagatggcagatctggagaagaggtgtgagc |
| | gcagactgcaggactgacccctcacttgaccagtcccattcagatccggcttggacaggcacctgagatggtgccaaagtgc |
| | agctgactcttcccacgacagccctgcccttcccatgaggcaggctcttcagtgagtgtttgaacgtaattatgtagttttc |
| | tgtttaattgaaaaagagagctatgcctttttttctttttgg |
| 223945_x_at | caacattctcttctcaaacactgaccaaggaacagaggaagatgcagtcagagaaagtagcaggatagagacgccgagagag |
| SEQ ID | gagtatatgtgggtcacagcagtgagctcccacccgccttgcagtgaagatgtgaccccaggagagggagtgtctccttcca |
| NO: 334 | ggtgctagctctggacagcagctgattttaggcaggaaagtttcttcatcgttgtcctccctgctggtcacatgagtttacg |
| | attccttagaagtgtctcccacagggtggcaggactgggagaatctctgaggcgtgtcttccaggccctcccacagcttgtg |
| | ccctccacagtgtaggcttgaacacttggcctcatgcg |
| 224153_s_at | gcagaacatatgtaaccagaaggacaaatccacccttcccttttgtcaagcacatctgtgtgaagagaccaccaacaggctt |
| SEQ ID | tgtgtgagcaacaaggctgtttattcacttgagtgcaagtgggctgagtctgaaaacagagtcagtgaagggagatggggaa |
| NO: 335 | ggggttgctttataggagttgggtaggtaatggaaaattacagtaaaaagtggttatctattgttagcagaggagggggtca |
| | caaggtacatggtggggagatgataagacccattgtccagaagaagactgtcacaaggtcgattgatcagttaaggtaggac |
| | agggacaggtcacaatggtggaatgttgtaatgttggttaatcagttaaggcaggaactggctgttttacttcttgtgtggt |
| | ttttcagctgccc |
| 224908_s_at | accaagcctggctttatgtatttatttctgttcatgcggaatgattggttcagaactgttcctttcccttccatgatgtcct |
| SEQ ID | tgacacagaaggttatgcctggctcccagtcaggcttcatacttttggtccatgtaagtgctacccgttgctgggggaggag |
| NO: 336 | tcatggtttatttggaaatgtcagttgcaatcatggttctgtcatttgactgcacagtatcagaggagcctgttaacctctc |
| | tgtgccttagtttcttagcccatgaaagagatcattgcctgacccagggactacctcaagggcttttgatgaggacaagtga |
| | cagtaggaagatgcaagagcctttagtaccaaggttctcaacactgactacatgctggaatgactgtgaagcttttaaaaaa |
| | tgttagtgcccactcttcccctgtacccccggacagttaaatcagaacctcagacagcaatatgccttgagatgccttgaac |
| | catgcttgaga |
| 225432_s_at | ggcgctgatgcgaatacagctcacagagaaacgcatgtgctattggagaacaggtctttgtggagatctaaaggcagtgatt |
| SEQ ID | gatttcacagggagctctaatctctgtgattacatggtccttcaaactcccaaccaaagtgagaaaagcggcatgcagtgaa |
| NO: 337 | atgagcagtgagcagccctttagcaaaatcgccctccagtccttcctggagatgccttcagccagcatcccagactccacag |
| | ttatttatgaangatgtcgtgattctccctccacctgacagtttgtaagagtgaaagagcatctaacctgatgctcttggag |
| | agagataacctgtctgtcataacttaaaggatgagaaaatgtggtgtagctattaaagattcatgcagtcccaaaaggcact |
| | gtcctgggatgatgagagattataaggtgatttcataaaaggaatccaaccctgtgcc |
| 225502_at | gaagccttactacaattccaaaaatcatcatggttggaaatttgggaggagattatttgtgaacttgttacccttttggtaa |
| SEQ ID | tggtggactaattgctgtatagttatttttgttttattattactgttacattaatttaacatgcatttatagaagaatacat |
| NO: 338 | tcaaagcactgatgtaggagatacacggtacttggagcagtcagccaaaaatcacagatactgctttcacttaaatggaaac |
| | aattctccgataatgctttgctttttttcttatgtcactcttgtgtactatctatttttctcctctctgggaccaagtttct |
| | ttttataaagcaataatatctctgttttcatttcagaacatt |
| 225736_at | cctctggatattgatgcctcgggtgtggttggactgtcatttagtggacaccgaatccagagtgccactgtgctcctcaacg |
| SEQ ID | aggacgtcagtgatgagaagactgctgaggctgcgatgcagcgcctcaaagcggccaacattccagagcataacaccattgg |
| NO: 339 | cttcatgtttgcatgcgttggcaggggctttcagtattacagagccaaggggaatgttgaggctgatgcatttagaaagttt |
| | tttcctagtgttcccttattcggcttctttggaaatggagaaattggatgtgatcggatagtcactgggaactttatattga |
| | ggaaatgtaatgaggtaaaagatgatgatctgtttcatagctatacaacaataatggcactcatacatctggggtcat |
| 225853_at | acatatgtcggctaccgtgtctggccgttcacatctttggccactatttgcttgtgaaaaggtataatgaggtggtacttat |
| SEQ ID | catttttactgtgtctcatgttttgtatatttttgtttcatcaactaagatgcactgtaacatctctgaaatctggatatat |
| NO: 340 | tatcaatggtttatcatagttttgttagcaatacactgtcttttagtggtgcctaaaataatggtatagttgtgaggtgatc |
| | ttagatttgatgaagcacagtatgcaggtaggcctaatgggggaagatggtaatataaaagcaagaagtattttttttttgt |
| | aatgactgaaagctgttctgtggatgacctaccctttcctttaaacacgattctctcacttccaactccaaacttgctcaac |
| | taat |
| 225882_at | gcaccttacatatttgatgctcaggaagaaagtgcaagaagatccctaatagaacattaaagattcttaaaagtttttgaag |
| SEQ ID | taggctgcttggggtgaccagccccacccttacatctccatagttggtacagttagcttgtagcagctgaagctgatgcctg |
| NO: 341 | gagatcactgtctgttggtctgatctcagtatcatactgagacacctcccctgagccttacctacttaaattggtaaccgtc |
| | ctaccaaaatttgtcaattttaatcaagtgaggcaagttgcaagggagccagagatacgtgaaaaaagaaaagcagaaatac |
| | tgatactttctaagaaagaagttgtaataatttctttggcacattgacttactgatatcttttgaaatgcatagangactct |
| | tgtgaaccaaagagagcagtagtggttattccccggcgcaataaaaatgcc |
| 226043_at | agtgggggggtggtcagtggaagctggggaggcttcactcagctcaaccctgcagaccccccatatgagaacccccctcctc |
| SEQ ID | ccaccagctgggggagatgacatcactgctctctccatggggagggggctcacagggcaccaagttcgggatcctaggactg |
| NO: 342 | gcaggacctgctggagcctgagatgggccccaggggcctgaggttgcctcctcctgtccagacccagccttggcacctccca |
| | agactgccaagggccctaccatggccccacagcccccagggctggcagttccatctaggagggtgcctctaggccccactct |
| | caggctaggatggagaggcagatctgggcctggngaggtgacctgcttctggaggggcagggccgcacccgagagcagggac |
| | aggtgcccgaacacagggtctccaggacgtagcgcccccccgcatacttgaatgtatgtgcgtatttattgctcacgtctgt |
| | gccatgttgtcaatgggtcctttccaacccaagaggtacatttg |
| 226778_at | gaaactctggaaatcacgtgtgtggggagatggggacgcttcccatgttgtggggagctctgtggctgtgatggctgcagtt |
| SEQ ID | gccgtgcctctgttggaacgcnaagtgcctgcaactcacgtcaatcatagaattgtgacgcacagttggcaaaatagttctt |
| NO: 343 | tatgctatttctcaaaantttgaggacaaacccagattgggattggaatatgcactgtaaatcaaatttttcttatctacaa |
| | agactaatgtaaaaatgattttttcttctgtgcctgattaaattaactgtggtttttaatataaatatttattggtgtgctt |
| | tgggagaaaaattatcttttcttgaaagaanttatcaaagcaaatttattatcttcacaagttaatgggagaatgtggtttt |
| | gattctgggtgtttgaattgtgtaaacacacagcttccttgtg |
| 227035_x_at | caacattctcttctcaaacactgaccaaggaacagaggaagatgcagtcagagaaagnagcaggatagagacgccgagagag |
| SEQ ID | gagtatatgtgggtcacagcagtgagctcccacccgccttgcagtgaagatgtgaccccaggagagggagtgtctccttcca |
| NO: 344 | ggtgctagctctggacagcagctgattttaggcaggaaagtttcttcatcgttgtcctccctgctggtcacatgagtttacg |
| | attccttagaagtgtctcccacagggtggcaggactgggagaatctctgaggcgtgtcttccaggccctcccacagcttgtg |
| | ccctccacagtgtagacttgaacacttggcctcatgtgatccttc |
| 228376_at | ggtcaggggatcagacactccagttgggtcttttatataaattaaatggcaaaaggctccatacccttctccttctttccta |
| SEQ ID | ccctccactttatctgcaaaatgggaatgatgataacacccacttcatagaatggtcatgaagatcaaatgagagaataaaa |
| NO: 345 | gtcaagcacttagcctctggtgcacaataagtattaaataagtatacctattcctccttttccttttttaaaaataatatta |
| | ccaaatgtccagcttatacacatttacaagacttagctagtgggctatgttagagctactaaaagatctttgacaagctaaa |
| | actaagatgcaatgaatgaggtgtaacgaacaagagagttttaagttcagaaatggttacagaagtataagacagctgtgtg |
| | ggtgttttttggtttttggtttctggtttacaatctcgtcattcaacaaagatgggagttttatagaactaaaagcaccatg |
| | taagctactaaaaacaacaacaaaaaaggctcatcatttctcagtctgaat |
| 228604_at | gaaatggtggtttccttctgataagggatacagtggaccatgtcgcttatatatcaccctactaggggatataattttccct |
| SEQ ID | tctaagcaagggaaatggcagtgctaaatagttttgtaaagtttttgaatgagaagcttttaggtaaagataaactgtcata |
| NO: 346 | atatttttcagatgcatttgcattgactatgggagaaaaattagggggaaatgacagtcatggtgtttttttttaattttaa |
| | ggactgaaattctgttacatgatgtatgacaatataaaaacatacttagttttatactaaatctttttttaaggtcttggca |
| | tttaatataaagcaaattcacacgttttctaactttccataagttccaaaaagggaaagaagaaacctcagtcttgaaattt |
| | tgatttttaaaaatcatgacactgttttaccatgaaattgagtagctaacttttggtaacacct |
| 228786_at | cacacgcagatcctgcactgtactgtccagtcaactgacttcagcaaagatgtctgggttcagcagcgagttgctgtgtgag |
| SEQ ID | gccaccagaatggaggtgatatcggcctcagtgctcatcctggaggtggagaagtggtcagagtcctctgtggtgaaatggc |
| NO: 347 | cctacaccaaagtgggagacattcagaatcgaggcgaaattggcctcagtgctcctcttggaggcagagaagcagtaggagt |
| | cggtggtgaaatggccctgtgtgaatgtggaagaccagcagattggaggtggaattggcctcagtgcttgtcctggaggtgg |
| | agaagcggtcagagtccttggtggtgaagtggccctgcacaaaacgttgctcccccttggccggtgaaagaataaaagaaag |
| | ctggccncttgcatttcngtttcagttnanagaanacatgttttggaaatagtatcttctagatctgtctttttctcttctg |
| | atttaacttagtaaagctaagttttatctcttc |
| 228812_at | gagacttcattggtgatacactcaatttttactgggtaattagctaataatgttggtcactgtctcacagttcaagtagctt |
| SEQ ID | taagatgatgtggcaaggaaaacacaaagcttttgggtaaccagcgttcttaaatgtatggtttttgaccaggtgaaccctt |
| NO: 348 | tagaagtgatttctgttttaaaagtatgtacttaaaatacctttggctgtgatgaatgtagatcccagcagaataccaaaat |
| | cctattttttttgactgagtatttgtagatgcttaatgactgaaatgaatttggaggcactgatgaaagtgatttttttnaa |
| | gttctcaggtactgttcaattat |
| 229598_at | atttttcttagctaaatctggcaactgtgnctatttcattgaaaacctgaaagtgtacaaagaaggaagaagcagaatctgc |
| SEQ ID | catatgagtaatagaagtgagcaggcccaggactccctaagtcaagaaaccaagaggcgtcattacggaaaagagtaactca |
| NO: 349 | ccctgtgtgctccttggtagttctccctcagcgatgcccccatgttatgaatggggaaaagttcactgaagggttcatagtg |
| | aagaaactttttggatgatttctgttggtgggttttggataccttcaagggatcagaaaataatatacttaggaaattttgg |
| | taatgtcatcattactctctacattattattatgacggttacaattgttaaatctaggtggtgggtatgtgggttatattgt |
| | acatgatttttaacttgtctgcatgtttgaaa |
| 231229_at | gcacgtccaaggtgatcctgagggctgtggcggacnaaggggacctgcaagtatntgtccctgnncaccctgaagaaggctg |
| SEQ ID | tttccaccacgggntacgacatggcccgaaatgcctatcacttcaagcgtgtgctcaaggggctggtggacaagggctcagc |
| NO: 350 | aggtgaccggcangggggcctcaggctccttcaccctgggcaagaagcaggcctccaagtccaagctcaaggtcaagaggca |
| | acgacagcagaggtggcgctctgggcagcgcccctttggacagcacaggtcactactgggctccaaacaggggcacaagcgg |
| | cttatcaagggggttcgaagggtggccaagtgccactgcaattaatgaggcaggccaggcaagcagtcaggggtgccaagan |
| | cgccattggctcagtgcagtgggaa |
| 232019_at | cctcggctcctcatctgaagagaagttgaacttaactgctaagttgctctggctctaatgtttcatgaggctgtggagcgtg |
| SEQ ID | atatagatgcggaagaggttagaagaccaggntgccggccnagctgtcctgtgcctgccacatctggccataccaaatccat |
| NO: 351 | cgcatgcttcaggaaagggaggggatgctgtgacagcaaactcagcccaggagcttgttggggaaggagaaagggcaagtcc |
| | ctcgtggtccacagcaggcaccccccaagcacctcgataaccagtggcaggtaggctgaagtgaaggaagcctctgtcagga |
| | ggtctggcgcccagtgacagacaagacagctgtgagggagctggggcagccctggctaaaggaatgtctgagtaacttgtga |
| | aactggaagcctctgcctctgaatgcattccaaga |
| 232311_at | acataccttgggttgatccacttaggaacctcagataataacatctgccacgtatagagcaattgctatgtcccaggcactc |
| SEQ ID | tactagacacttcatacagtttagaaaatcagatgggtgtagatcaaggcaggagcaggaaccaaaaagaaaggcataaaca |
| NO: 352 | taagaaaaaaaatggaaggggtggnaaacagagtacaataacatgagtaatttgatgggggctattatgaactgagaaatga |
| | actttgaaaagtatcttggggccaaatcatgtagactcttgagtgatgtgttaaggaatgctatgagtgctgagagggcatc |
| | agaagtccttgagagcctcc |
| 235391_at | atttcacggcaaagctttagaggtctacactgctgcctaccagaatatacaaaacattgatgaagatgaagatttagaggtt |
| SEQ ID | ttccnaaattctctgtatgcaccagattattcatctcgtttagatattgtaagagcaaattcaaagtcacctcttcagagat |
| NO: 353 | cactgtcagctaagtgtgtatctggaacaggacaggtatccacttgtcgactaagaaaggatcaacaagcagaagatgatga |
| | ggatgacgagttagatgttacagaagaagaaaattttcttaagtaaactacacatttccattttcatcataaatgacttgaa |
| | atccacaatgactaaattgtagaactttatactcactttgctatgttaagcctcaaagtgaagtccaactgg |
| 235421_at | ataccttcacaatctgaactttctctagatgggcacagatcatgaataactcagaaaccattagcatattgtgtgcttaatt |
| SEQ ID | aaggctggaaagtgctgaggctttttgcaggcgcagttagcaggcgaactggtcagcagggtttctggggaacgtagatttg |
| NO: 354 | cgtgacaggggccctgtgttgctaaggaattaatgcaggcactgagaaaagtctcagcgtcagacactcctccacctcccgg |
| | gtcctgcttccctccctctcccctttccaggcaactcacagtttaggggtgtggttgtgtgtgttcaagctcttgggtttta |
| | catataaatgcctgagacttttctgcagggtttaaaaactaacatcttgtcttacagctctttta |
| 235473_at | ggaaatcaagatctgcgtgctgggtatgttcattgctactggggtgtcattgcttctaggccttttcaactgacagggtcag |
| SEQ ID | taatggagtaaaactcattgtaatcgtgtaacttatgtgggttaagaaattgaactttgctagtaccccagaagctccttta |
| NO: 355 | tgtcccctcctgattttaacgcttaatcaccttcatcctcagttttatggtaatcacttgtctttgcagttttagtctgtaa |
| | ccatgcatccctaaataaatgtttagattgcctattttaaaaatttaatataattggttcaatgtgtgcatttcatgtatga |
| | cttcttttatgtctgtgatgttca |
| 235688_s_at | tgaggcagtgcatgttcttggcccagagtaagtgcttagtgaatgctttctaactccgaaccccagccacatccagggactg |
| SEQ ID | ggtgttgagcaaaaggggccttcaagatgttcaaggcacttggattttctcctgtctctcatcggcttttcttaacgggcct |
| NO: 356 | cagtgggtgcatgtgattatccacgtttcacctatgaaacatgaacagaggagactgacttatcagtgattcttccgcgggt |
| | tcggacagggcctcgattctgttttaaactccagtagtccctagaaattgtagctccctctagttgtggcaataggtgtggg |
| | tccttgtgcttgcttttggcaagtttntgagctacacagggcctccattaccgtcactggtgaaatgcggctcacctccc |
| 236203_at | atgtcaggtttgtacctaccacatttaaaatagggacttgaagaattaaacattttattacaaatgaagcacttcatgcaca |
| SEQ ID | gactggcacatagtaagtagtcgataggtgttaacaatttgtgttattgttattttctggagtccaactaacaaatcccaca |
| NO: 357 | gtgaatgacatcacagggatgcaaccaacaagatccagaatatggaaacttctactagataaacaactccatttcttcagca |
| | acaattcaagagagagagagaagagaagctatacattttaaaaggctgaagaaatatatgaaccaaatttgtatgaggcaat |
| | cagaaaaactgacaccgactgtattaaggaattatctaattttagtgtggtaatgagattgctgttatgttttctaa |
| 236627_at | aactagatttttgaccctcactcatctggctttccttgctatagacatgctttgcatacccagcttctagccacatcttccc |
| SEQ ID | taagaaaatttcancctggaatcagttccaaaagaaaatcttaagagatagagtatggatctgcatgttgtcagctacctaa |
| NO: 358 | agcagcaaaaattcctagatgagaataaaatcagggaaccttgcagtttaaggcacaccttaaatattaaatttttaatcct |
| | tcaatgcatccagccaaaacaagaaattgcaaatagctaccatccatcaatgcaaggttttt |
| 236681_at | gaaactcatgtgtcctcatggatcgtggatgccttcatttcttgagctctcaatgcagacatttaaatggctgcaatcagta |
| SEQ ID | gagtgacccgcggatggcataaatgcacctccttttcttggccttggatctatgggtctgggattgtggtcatctcctcaat |
| NO: 359 | cctcaaaaagaggctgaatcaatgtggccgtgggtgggaacttacatacagaacccaatgaagaacttgactgtctaaacaa |
| | gggggcctcgcatggagctgtaaagcatctaacaaatatgaaaaatgtgaagttccaaggtccaagaagaaaaataatgatg |
| | tttctgaaagtgatgataaataattacttttaaagtgctgcatatttatacaattgagagattatttttgtaaatgcaatgt |
| | ctgtgagctgggatacatgggcagtgcttcagac |
| 237439_at | tatttttaatgccttttcctgggataagcattaaagatgnccnaaaagaaaanaaaaacnaaagaatgatagtgatggtaag |
| SEQ ID | gcaagattctagcaaagagagatgggagataaatggctgagagttcaggtgaatat |
| NO: 360 | |
| 238925_at | ggcagcagtggcatggacaagcaactgctaattcgagacttactattggcttcacagcacaccctacagtgagcagggtgat |
| SEQ ID | gagaccgtggaacagctacctgcttttggttaagattgccagatatcattaggacacagtagcagcaagaatgctgattttg |
| NO: 361 | tagtgttagaaaatgaactcagcttgtttttcctaattttgaaggtggtatatatctgcaaacatttaaaatggtntaatat |
| | catataaaatgtcaaaattagcacagtccctggatctcaaagaataggtaatattgacttggatgttctttatgctctctga |
| | agaaaggtctagggaaagttcttgtttgcttgattgatcaagagtgccacggaagaatgtgagcgctgtggagggtggagga |
| | gggtgtcacttcagatggggcaggcaca |
| 239486_at | taaagttgggcagtaatcttaattatgatggaattatcattatgctaagtaattaactttacctagtttgttttacaactag |
| SEQ ID | aacctgccctaaatgttgaatatcttcctagcaagaaacagtctgtcattttacttacacgatgtctaaccaaaccataact |
| NO: 362 | ttacataaactagtcgtttcggtcaaatagaaaaatgtgtgaatgccataaaaacaaaaattctcagttaaatgatactggg |
| | aaatagggaagacagcaaagtgagacttgggctcanggatgngttcaggaanaaaaaaaaagaaagacccctgagtaccatt |
| | aatattcctcagaaattattatttcaaaaggaaatatttctgtattataaatttttcatgagcagccattatgaaatctcac |
| | aa |
| 241670_x_at | ttgcctgcggagcacgggtgggcattggatgtaaggtattcctaacttccccttccccagtccttggcgtcctctctancca |
| SEQ ID | tcccaccccaggtcttctgcagcttcaccctgtcctccaagtcccctcgcccagctgcgagncaggagaccccgnccggant |
| NO: 363 | ccgccagcaggnccttctttcgcagggcgcanacgcacgatnccgggatcgggtgcaccacngcgaancccangcgggagac |
| | ggcaggagcagctcaggtaaccaggggaagcttgcgtgcc |
| 241671_x_at | attctgaagccagaccgtctttcctcacagcnnnnnngnnncnnnnccgngggcacgcaagcttcccctggttacctgagct |
| SEQ ID | gctcctgccgtctcccgcntgggnttcgcngtggtgcacccgatcccggnatcgtgcgtntgcgccctgcgaaagaaggncc |
| NO: 364 | tgctggcggantccggncggggtctcctgnctcgcagctgggcgaggggacttggaggacagggtgaagctgcagaagacct |
| | ggggtgggatggntagagaggacgccaaggactggggaaggggaagttaggaataccttacatccaatgccca |
| 243010_at | gaaatacacccactctcttggaataatgacgtaccactcagttggaccctcaagagtcactgctttgtctgtgctggtagtt |
| SEQ ID | tgtgagaagtgacccgcacgcttccatttgatgcatttgatgtgagtgaatccatacatttgaatgtcattgtccttgagac |
| NO: 365 | cctacatgtgcagtttggctcatctcattaaagatgcttgatgtaataattggttagtttccttttattttcctgcaggctt |
| | ttccatgagtattatttttttcaaagaacaaatctgtatggcttttccccatctccatattttgttttgctatgaattgctt |
| | tgctttggtgaacttgtcctagtatgcttgcctcacaaacgttttagccattgtgaattttcttcatctctgtaaatagttc |
| | atctgtgcttctccctgatgacgttttattttttttcccctgtaagcaaccgaggtagaaaaataaattgtttaccatggan |
| | anannnnntgctgccgtctcttagcctgacagtgtcctgttctc |
| 243502_at | ttaatttctgtgaagagtgcccctggtgtttcatcttggcctgttttgatgagaatgttatcntttgtgtctggataacgcg |
| SEQ ID | tcagcttcttaaagtacatataaagatattctgtcaccnccccacatgcacacacttttaaaatctatttttattctcttgc |
| NO: 366 | taaagttgtaattatgtcaagaattttccagctctaactgccttcttagtacatgtctttctgcctttgaagcatatgagtt |
| | tgccaaagtcattctcccctaatgacatattgtggactta |
| 244393_x_at | cccagccgctataacttttaacaattcccatatgtcctttattccactaagatgagtgcagtatatatttccatctgtccaa |
| SEQ ID | ggcttcctaaatgtagccaangccaagccaacaccagtcacatgatcnaaatcaaagggcatttggggaatccaggctgtga |
| NO: 367 | ttcagggaagttccaagtgtctgatgaagtgtttgttttacatctttgtgtcccttgcaggtctagcactgtgctatgtagg |
| | taacatgtgctcc |
| 91684_g_at | agctgcgcaagatccaggcgcggatgggcgtgttcgcgcaggctgacggctcggcctacattgagcagggcaacaccaaggc |
| SEQ ID | actggctgtggtctacggcccgcac |
| NO: 368 | |

TABLE 12
105 PS gene list, target sequences

| | |
|---|---|
| Probe Set ID | Target sequence |

| | |
|---|---|
| 1555852_at | ccattctgagtacttctccgcaaaccctttgtttcattaaggactgttttacatgaagggtgcaaaagtaggataaaaatgag |
| SEQ ID | aaccctagggtgaaacacgtgacagaagaataaagactattgaatagtcctcttctctacccatggacnttggnatttttata |
| NO: 369 | ttngattttaaggaaatataacttagtagtaaagagatgagcattcaagtcaggcagacctgaatttgggtcaaggctgcgcc |
| | actcaaaagctatatgacctctatatgagcagcttattcaacctcttttaacctccattttgtcatctgtagaatgatgataaatgccta |
| | gctcagaaggattcc |
| 1556340_at | tcccccctggacaactgtatacccaagacagtccccagggcctcgcctgtctcccccagcccccgntaagcagtccagactcc |
| SEQ ID | aaccccaactgctggggtccatcatcccctataccaggggctatcccctcaacttggagggctggccaaa |
| NO: 370 | |
| 1558034_s_at | catctaccctgataacaccacagattttcaaagagcagatgacaaagtatatccaggagagcagtatacatacatgttgcttg |
| SEQ ID | ccactgaagaacaaagtcctggggaaggagatggcaattgtgtgactaggatttaccattcccacattgatgctccaaaagat |
| NO: 371 | attgcctcaggactcatcggacctttaataatctgtaaaaanngattctctagataaagaaaaangaaaaacatattgaccga |
| | gaatttgtggtgatgttnnctgtggtggatgaaaatttcagctggtacctagaagacaacattaaaacctactgctcagaacc |
| | agagaaagttgacaaagacaacgaagacttccaggagagtaacagaatgtattctgtgaatggatacacttttggaagtctcccagg |
| 1558290_a_at | ttttgcatgtctgacacccatgactccacctggaccttatggctccacccagaagcaattcagcccaacaggaggacagcttc |
| SEQ ID | aacccattacgatttcatctctgccccaaccactcagcagcaagcacctgttacctgtccacccccaccccttcccccaaact |
| NO: 372 | gcctttgaaaaatccctaacctatgagctttgaataagatgagtacgaacttcatcgccca |
| 1559263_s_at | gcttcctttgagaacaagtttgagttttagtaagctgtaaagctgttttatttgattcactgtgaataagacaagtacaaaaa |
| SEQ ID | aaaaagtgaaaacacagcacatttcaagtgtaatcccctttggaatttttccaaagtcttagtatatgtctttgtgacagaaa |
| NO: 373 | aacatagatgggacaggtaaatgaaatgttcctagttgcatagctgttaacc |
| 1559584_a_at | attcctgggctcgagagagccactcatccccatctctcagagtgttgggattacagccatgagccaccatgcccggccaaact |
| SEQ ID | tcagccttgaatattagtgattttgccatattagtttaccatctgtacaattatttgctttatntttttctttaaattggctc |
| NO: 374 | attattcacttaatgaaattattgcaacaggaatcttttgtatcacttctgtaaatggaaaaccagtctcatttgccatcaat |
| | agaaggt |
| 1561042_at | aaatgcagataaaggctacctctgaattctcaatagattcatcatgtttgctcttaagtgtagctgtccacactg |
| SEQ ID | |
| NO: 375 | |
| 1562031_at | atgttcactgtatgtgccaagcctaatatgagagctatgtattatagagtttatgctacagccctaccttcaggaaacttatc |
| SEQ ID | tactggacaaacaaaaattttcaaatatacaaaaaattctaaatcgaacattgtaattatctagcataggcaaatatagacag |
| NO: 376 | taacagacaggtttacaattattaagaaagggcagccagg |
| 1563497_at | taccatagcaagttcttatcctttgttaatatattataaatacgacttttttcctttacctgtcacagtaatatgccaatttt |
| SEQ ID | tggtgtcgctttagtaatttttttgggggggactgtcaaatttcagtaaactttataggctatagtttacaatttaaggtctt |
| NO: 377 | tcctcagttgtctcctaaaaaattttgtggaaatcgaaatttcttttcaattgatcgtcttgtgcagccctttaaaaaatgcg |
| | ttttgcattcatgtctttgcagtacaaatctgtatctttcattttgggagtgactgtcatagatgggtaatataaagtgacaa |
| | cacacacaccagggccagattgcgtgggttctagctagggacctagagctggttacttgatttctctctgttccatttactat |
| | ctg |
| 200904_at | tgtgccttcattcatgggttaatggattaatgggttatcacaggaatgggactggtggctttataagaagaggaaaagagaac |
| SEQ ID | tgagctagcatgcccagcccacagagagcctccactagagtgatgctaagtggaaatgtgaggtgcagctgccacagagggcc |
| NO: 378 | cccaccangggaaatgtctagtgtctagtggatccaggccacaggagagagtgccttgtggagcgctgggagcaggacctgac |
| | caccaccaggaccccagaactgtggagtcagtggcagcatgcagcgcccccttgggaaagctttaggcaccagcctgcaaccc |
| | attcgagcagccacgtaggctgcacccagcaaagccacaggcacggggctacctgangccttgggggcccaatccctgctcca |
| | gtgtgtccgtgaggcagcacacgaagtcaaaagagattattctcttcccacagataccttttctctcccatgaccctttaaca |
| | gcatctgcttcattcccctcaccttcccaggctgatctgaggtaaa |
| 200905_x_at | aaagcctgagacagctgccttgtgtgcgactgagatgcacagctgccttgtgtgcgactgagatgcaggatttcctcacgcct |
| SEQ ID | cccctatgtgtcttaggggactctggcttctctttttgcaagggcctctgaatctgtctgtgtccctgttagcacaatgtgag |
| NO: 379 | gaggtagagaaacagtccacctctgtgtctaccatgacccccttcctcacactgacctgtgttccttccctgttctcttttct |
| | attaaaaataagaacctgggcagagtgcggcagctcatgcctgtaatcccagcacttagggaggccgaggagggcagatcacg |
| | aggtcaggagatcgaaaccatcctggctaacacggtgaaaccccgtctctactaaaaaatacaaaaaattagctgggcgcaga |
| | ggcacgggcctgtagtcccagctactcaggaggcggaggcatggagaatggcgtcaacccgggaggcggaggttgcagtgagc |
| | caggattgtgcgactgcactccagcctgggtgacagggtgaaacgccatctc |
| 201009_s_at | atactgaggtggatccctgcatcctcaacaacaatgtgcagtgagcatgtggaagaaaagaagcagctttacctacttgtttc |
| SEQ ID | tttttgtctctcttcctggacactcactttttcagagactcaacagtctctgcaatggagtgtgggtccaccttagcctctga |
| NO: 380 | cttcctaatgtaggaggtggtcagcaggcaatctcctgggccttaaaggatgcggactcatcctcagccagcgcccatgttgt |
| | gatacaggggtgtttgttggatgggtttaaaaataactagaaaaactcaggcccatccattttctcagatctccttgaaaatt |
| | gaggccttttcgatagtttcgggtcaggtaaaaatggcctcctggcgtaagctttt |
| 201010_s_at | gtgttctcctactgcaaatattttcatatgggaggatggttttctcttcatgtaagtccttggaattgattctaaggtgatgt |
| SEQ ID | tcttagcactttaattcctgtcaaattttttgttctccccttctgccatcttaaatgtaagctgaaactggtctactgtgtct |
| NO: 381 | ctagggttaagccaaaagacaaaaaaaattttactacttttgagattgccccaatgtacagaattatataattctaacgctta |
| | aatcatgtgaaagggttgctgctgtcagccttgcccactgtgacttcaaacccaaggaggaactcttgatcaagatgcccaac |
| | cctgtgatcagaacctccaaatactgccatgagaaactagagggcaggtgttcataaaagccctttgaacccccttcctgccc |
| | tgtgttaggagatagggatattggcccctcactgcagctgccagcacttggtcagtcactctcagccatagcactttgttcac |
| | tgtcctgtgtcagagcactgagctccacccttttctgagagttat |
| 201891_s_at | tttataattctactttgagtgctgtctccatgtttgatgtatctgagcaggttgctccacaggtagctctaggagggctggca |
| SEQ ID | acttagaggtggggagcagagaattctcttatccaacatcaacatcttggtcagatttgaactcttcaatctcttgcactcaa |
| NO: 382 | agcttgttaagatagttaagcgtgcataagttaacttccaatttacatactctgcttagaatttgggggaaaatttagaaata |
| | taattgacaggattattggaaatttgttataatgaatgaaacattttgtcatataagattcatatttacttcttatacatttg |
| | ataaagtaaggcatggttgtggttaatctggttt |
| 202546_at | gatctggtgcggaacctgcaaagtgaggtggagggagttaagaatattatgacccagaatgtggagcggatcctggcccgggg |
| SEQ ID | ggaaaacttggaacatctccgcaacaagacagaggatctggaagccacatctgagcacttcaagacgacatcgcagaaggtgg |
| NO: 383 | ctcgaaaattctggtggaagaacgtgaagatgattgtccttatctgcgtgattgtttttatcatcatcctcttcattgtgctc |
| | tttgccactggtgccttctcttaaagtaa |
| 202688_at | ctctacctcatatcagtttgctagcagaaatctagaagactgtcagcttccaaacattaatgcaatggttaacatcttctgtc |
| SEQ ID | tttataatctactccttgtaaagactgtagaagaaagcgcaacaatccatctctcaagtagtgtatcacagtagtagcctcca |
| NO: 384 | ggtttccttaagggacaacatccttaagtcaaaagagagaagaggcaccactaaaagatcgcagtttgcctggtgcagtggc |
| 203915_at | gattatcaattaccacaccatctcccatgaagaaagggaacggtgaagtactaagcgctagaggaagcagccaagtcggttag |
| SEQ ID | tggaagcatgattggtgcccagttagcctctgcaggatgtggaaacctccttccaggggaggttcagtgaattgtgtaggaga |
| NO: 385 | ggttgtctgtggccagaatttaaacctatactcactttcccaaattgaatcactgctcacactgctgatgatttagagtgctg |
| | tccggtggagatcccacccgaacgtcttatctaatcatgaaactccctagttccttcatgtaacttccctgaaaaatctaagt |
| | gtttcataaatttgagagtctgtgacccacttacc |
| 204057_at | aattgtcggattttccatgtcctcctttctcctttgtgcccagcctgagtcagcaccatcccgcattcagaacctcccagtga |
| SEQ ID | aagggcagccttcattttgagaaggtggaaggtgttagggtttgggagacagctcatccaatctcccaagtctcatggtggat |
| NO: 386 | ttgtgactgtgagagtttccggtttaaaatctgaaaagccagatatgcctgtttccttttcccagcaccatgcctgtggaggg |
| | gacagtcagacccagaggtcctttacgtgtggatggagttcacaggcgaatagaggagaggaccaggggacgtggcttgtccc |
| | ttttgtccaacaaagcattatatttttaagaatggcagncctgtttgctgaagtgttcataagataacaataggcttgaatct |
| | ccaattc |
| 204078_at | aacttaagatggacacagctgactggacccccatcctgcctcacccatgggtgctgcaccccagacccatcctgccacttcta |
| SEQ ID | tgtctctggaccacaggatggtggtggcattgcaggttggcaagtgggctgatggggtccgccctcctcactgctgagctcct |
| NO: 387 | cacctggacagtctcctggacaaggagtttccagctgctggctggagtctcaggccaaattgcagagggtcctccagggtcct |
| | gaagagcactggactaagagtctagtggttccagggc |
| 204116_at | ttctggctggaacggacgatgccccgaattcccaccctgaagaacctagaggatcttgttactgaataccacgggaacttttc |
| SEQ ID | ggcctggagtggtgtgtctaagggactggctgagagtctgcagccagactacagtgaacgactctgcctcgtcagtgagattc |
| NO: 388 | ccccaaaaggaggggcccttggggaggggcctggggcctccccatgcaaccagcatagcccctactgggcccccccatgttac |
| | accctaaagcctgaaacctgaaccccaatcctctgacagaagaaccccagggtcctgtagccctaagtggtactaactttcct |
| | tcattcaacccacctgcgtctcatactcacctcaccccactgtggctgatttggaattttgtgcccccatgtaagcacc |
| 204233_s_at | gcaaacatccggaagtatcccaccaagaaacaacagctccattttatttccagttacttgcctgcattccaaaatgactttga |
| SEQ ID | aaacctcagtactgaagaaaaatccattataaaagaagaaatgttgcttgaagttaataggtttgcccttgcatctcatttcc |
| NO: 389 | tctggggactgtggtccattgtacaagccaagatttcatctattgaatttgggtacatggactacgcccaagcaaggtttgat |
| | gcctatttccaccagaagaggaagcttggggtgtgactgtggggaggactccatccacctcatcactggactgcatggggagg |
| | cagcagagcggggtcccctctgtgcttcgactactgctcctgtggcaggaggctttgggtggctcactactgaacaca |
| 204661_at | acagccacgaagatcctaccaaaatgaagcgcttcctcttcctcctactcaccatcagcctcctggttatggtacagatacaa |
| SEQ ID | actggactctcaggacaaaacgacaccagccaaaccagcagcccctcagcatccagcagcatgagcggaggcattttcctttt |
| NO: 390 | cttcgtggccaatgccataatccacctcttctgcttcagttgaggtgacacgtctcagccttagccctgtgccccctgaaaca |
| | gctgccaccatcactcgcaagagaatcccctccatctttgggaggggttgatgccagacatcaccaggttgtagaagttgaca |
| | ggcagtgccatgggggcaacagccaaaataggggggtaatgatgtaggggccaagc |
| 204724_s_at | caccagcaaggacggccaggacggtgctcccggcgagcctgggcctcccggagatcctgggcttccaggtgccattggggccc |
| SEQ ID | aggggacaccggggatctgcgacacctcagcctgccaaggagccgtgttaggaggggtcggggagaaatcaggctctcgaagc |
| NO: 391 | tcataaaattcaacgtgaggaagcaagtgacaaggacgcccgaagcacagtggacggtcatgaaggagcgggggtgtggcagg |
| | cgggtgacgtccaggagagggagcgcccctggctgcccctcggccgccgactggacgcgtgggccttgccagcgagcaccctc |
| | attgggctgtcgcctgacagcatacctcaaaaggccctagctaataaacctgtaagcccagcatttgagagaaggtagggtgt |
| | gtatatataaaaggttgtgtacaactccacgaggtgaaaaatattcagtaacttgtttgcatagcatttgtgtaaagactatg |
| | atctcatcccaata |
| 205159_at | gatttacaaaggtcctcccattgcaaagcagtgtttgtcctaatttatatattgtttttctagttcattttgtgtttccaact |
| SEQ ID | tttcatgtaaaattttaattatttttgaatgtgtggatgtgagactgaggtgccttttggtactgaaattctttttccatgta |
| NO: 392 | cctgaagtgttacttttgtgatataggaaatccttgtatatatactttattggtccctaggcttcctattttgttaccttgct |
| | ttctctatggcatccaccattttgattgttctacttttatgatatgttttcataagtggttaagcaagtattctcgttacttt |
| | tgctcttaaatccctattcattacagcaatgttggtggtcaaagaaaatgataaacaacttgaatgttcaatggtcctgaaat |
| | acataacaacattttagtacattgtaaagtagaatcctctgttcataatgaac |
| 205204_at | agatccgagtgcactcgcgaggcaacctctgggccaccggtcacttcatgggcaagaagagtctggagccttccagcccatcc |
| SEQ ID | cattggggacagctccccacacctcccctgagggaccagcgactgcagctgagtcatgatctgctcggaatcctcctgctaaa |
| NO: 393 | gaaggctctgggcgtgagcctcagccgccccgcaccccaaatccagtacaggaggctgctggtacaaatactgcagaaatgac |
| | accaataataggggcagacacaacagcgtggcttagattgtgcccacccagggaaggtgctgaatgggaccctgttgatggcc |
| | ccatctggatgtaaatcctgagctcaaatctctgttactccattactgtga |
| 205419_at | cagcccctgaagaaaattcacgtgaaatgacagaaacgcagatgatgatacattccaagtcttcaaatggaaagtgaaatgga |
| SEQ ID | ttgtattttggtttatagtgacgtaaactgtatgacaaactttgcaggacttcccttataaagcaaaataattgttcagcttc |
| NO: 394 | caattagtattcttttatatttctttcattgggcgctttcccatctccaactcggaagtaagcccaagagaacaacataaagc |
| | aaacaacataaagcacaataaaaatgcaaataaatattttcatttttatttgtaaacgaatacaccaaaaggaggcgctctta |
| | ataactcccaatgtaaaaagttttgttttaataaaaaattaattattattcttgccaacaaatggctagaaaggactgaatag |
| | attatatattgccagatgttaatactgtaacatactttttaaataacatatttcttaaatccaaatttctctcaatgttagat |
| | ttaattccctcaataacaccaa |
| 205668_at | ggaactctaaaccttgtgatgactactaacaaatgtaaaattatgagtgattaagaaaacattgctttgtggttatcacttta |
| SEQ ID | agttttgacacctagattatagtcttagtaatagcatccactggaaaaggtgaaaatgttttattcagcatttaacttacatt |
| NO: 395 | tgtactttagagtatttttgtataaaatccatagatttattttacatttagagtatttacactatgataaagttgtaaataat |
| | tttctaagacagtttttatatagtctacagttgtcctgatttcttattgaatttgttagactagttctcttgtcttgtgatct |
| | gtgtacaattttagtcactaagactttcctccaagaactaagccaacttgatgtgaaaagcacggctgtatataatggtgatg |
| | tca |
| 205696_s_at | ttcctgttctcttgtatagctgaaattccagtttaggagctcagttgagaaacagttccattcaactggaacatttttttttt |
| SEQ ID | tccttttaagaaagcttcttgtgatccttcggggcttctgtgaaaaacctgatgcagtgctccatccaaactcagaaggcttt |
| NO: 396 | gggatatgctgtattttaaagggacagtttgtaacttgggctgtaaagcaaactggggctgtgttttcgatgatgatgatcat |
| | catgatcatgatgattttaacagttttacttctggcctttcctagctagagaaggagttaatatttctaaggtaactcccata |
| | tctcctttaatgacattgatttctaatgatataaatttcagcctacattgatgccaagcttttttgccacaaagaagattctt |
| | accaagagtgggctttgtggaaacagctggtact |
| 205758_at | cagcccttgcattgcagaggggcccatgaaagaggacaggctacccctttacaaatagaatttgagcatcagtgaggttaaac |
| SEQ ID | taaggccctcttgaatctctgaatttgagatacaaacatgttcctgggatcactgatgactttttatactttgtaaagacaat |
| NO: 397 | tgttggagagcccctcacacagccctggcctcngctcaactagcagatacagggatgaggcagacctgactctcttaaggagg |
| | ctgagagcccaaactgctgtcccaaacatgcacttccttgcttaaggtatggtacaagcaatgcctgcccattggagagaaaa |
| | aacttaagtagataaggaaataagaaccactcataattcttcaccttaggaataatctcctgttaatatggtgtacattcttc |
| | ctgattattttctacacatac |
| 205890_s_at | gatcttaaagccacggagaagcctctcatcttatggcattgacaaagagaagaccatccaccttaccctgaaagtggtgaagc |
| SEQ ID | ccagtgatgaggagctgcccttgtttcttgtggagtcaggtgatgaggcaaagaggcacctcctccaggtgcgaaggtccagc |
| NO: 398 | tcagtggcacaagtgaaagcaatgatcgagactaagacgggtataatccctgagacccagattgtgacttgcaatggaaagag |
| | actggaagatgggaagatgatggcagattacggcatcagaaagggcaacttactcttcctggcatcttattgtattggagggt |
| | gaccaccctggggatggggtgttggcaggggtcaaaaagcttatttcttttaatctcttactcaacgaacacatcttctgatg |
| | atttcccaaaattaatgagaatgagatgagtagagtaagatttgggtgggatgggtaggatgaagtatattgcccaactctat |
| | gtttctttga |
| 206082_at | tgaaggatggtgactgcgccatggcctggatctgctgcagtgtcctttcctgtggaggctccactcaaagctggcatcctcct |
| SEQ ID | atgtcacctagagtgtgggtcaaagcaatacacctacatgtagaatgtgatgtcagaactcaaacaggctcaccaggcagtgt |
| NO: 399 | gcttcttccttgcatgaggatgcaagatgcaacagtttgtcttcacattggaaggacacccctggatgcccctaaccactaga |
| | cctgtaaaacttcactgcagtggccacttctgaatctctgtaaggtttatttatcttcacccctctggagagaagatgtttta |
| | ccaaagcctctagtgtaccgtcctcctcttactcatccatcccagtcaacatgatgttgtcaatgaaataaaggaatttaata |
| | ttctatagtatatccaggttctccagatctcttaagactgtactatagaggcctgggg |
| 206134_at | ctttctatattgttatcagtccaggaaacaggtaaacagatgtaattagagacattggctctttgtttaggcctaatctttct |
| SEQ ID | ttttacttttttttttcttttttctttttttttaaagatcatgaatttgtgacttagttctgccctttggagaacaaaagaaa |
| NO: 400 | gcagtcttccatcaaatcaccttaaaatgcacggctaaactattcagagttaacactccagaattgttaaattacaagtacta |
| | tgctttaatgcttctttcatcttactagtatggcctataaaaaaaataataccacttgatgggtgaaggctttggcaatagaa |
| | agaagaatagaattcaggttttatgttattcctctgtgttcacttcgccttgctcttgaaagtgcagtatttttctacatcat |
| | gtcgagaatgattcaatgtaaatatttttcattttatcatgtatatcctatacacacatctccttcatcatcatatatgaagt |
| | ttattttgagaagtctacattgcttacattt |
| 206540_at | ggatcaatgggtttaacttgggccggtactggacaaagcaggggccacaacagaccctctacgtgccaagattcctgctgttt |
| SEQ ID | cctaggggagccctcaacaaaattacattgctggaactagaagatgtacctctccagccccaagtccaatttttggataagcc |
| NO: 401 | tatcctcaatagcactagtactttgcacaggacacatatcaattccctttcagctgatacactgagtgcctctgaacca |
| 206574_s_at | gaaggatggcatcaccgttgtggactggccgtttgacgatggggcgcccccgcctggcaaggtagtggaagactggctgagcc |
| SEQ ID | tggtgaaggccaagttctgtgaggcccccggcagctgcgtggctgtgcactgcgtggcgggcctggggcgggctccagtcctt |
| NO: 402 | gtggcgctggcgcttattgagagcgggatgaagtacgaggacgccatccagttcatccgccagaagcgccgcggacgcatcaa |
| | cagcaagcagctcacctacctggagaaataccggcccaaacagaggctgcggttcaaagacccacacacgcacaagacccggt |
| | gctgcgttatgtagctcaggaccttggctgggcctggtcgtcatgtaggtcaggaccttggctggacctggaggccctgccag |
| | ccctgctctgcccagcccagcagggctccaggccttggctggccccacatcgccttttcctccccgacacctccgtgcacttg |
| | tgtccgaggag |
| 206666_at | aaacctctcttagatctggaaccaaatgcaaggttactggctggggagccaccgatccagattcattaagaccttctgacacc |
| SEQ ID | ctgcgagaagtcactgttactgtcctaagtcgaaaactttgcaacagccaaagttactacaacggcgacccttttatcaccaa |
| NO: 403 | agacatggtctgtgcaggagatgccaaaggccagaaggattcctgtaagggtgactcagggggccccttgatctgtaaaggtg |
| | tcttccacgctatagtctctggaggtcatgaatgtggtgttgccacaaagcctggaatctacaccctgttaaccaagaaatac |
| | cagacttggatcaaaagcaaccttgtcccgcctcatacaaattaagttacaaataattttattggatgcacttgcttcttttt |
| | tcctaatatgctcgcaggttagagttgggtgtaagtaaagcagagcacatatggggtccatttttgcacttgta |
| 207175_at | aaataacatacgcactcaacttccttttctttcttccttcctttctttcttccttcctttctttctctctctctctttccttc |
| SEQ ID | cttccttcctccttttctctctctctctctctctctctcttttcttgacagactctcgttctgtggccctggctggagttcag |
| NO: 404 | tggtgtgatcttggctcactgctacctctaccatgagcaattctcctgcctcagcctcccaagtagctggaactacaggctca |
| | tgccactgcgcccagctaatttttgtatttttcgtagagacggggtttcaccacattcgtcaggttggtttcaaactcctgac |
| | tttgtgatccacccgcctcggcctcccaaagtgctgggattacaggcatgagccatcacacctggtcaactttcttttgatta |
| | gtgtttttgtggtatatctttttccatcatgttactttaaatatatctatattattgtatttaaaatgtgtttcttacagact |
| | gcatgtagttgggtataatttttatccagtctaaaaatatctgtcttttaattggtgttt |
| 207651_at | ttgccttgtaattcgacagctctacagaaacaaagataatgaaaattacccaaatgtgaaaaaggctctcatcaacatacttt |
| SEQ ID | tagtgaccacgggctacatcatatgctttgttccttaccacattgtccgaatcccgtataccctcagccagacagaagtcata |
| NO: 405 | actgattgctcaaccaggatttcactcttcaaagccaaagaggctacactgctcctggctgtgtcgaacctgtgctttgatcc |
| | tatcctgtactatcacctctcaaaagcattccgctcaaaggtcactgagacttttgcctcacctaaagagaccaaggctcaga |
| | aagaaaaattaagatgtgaaaataatgcataaaagacaggattttttgtgctaccaattctggccttactgga |
| 208296_x_at | ttgagttctccttttaagtaccaatgatacttaaatttctcagaaatgtaatggtgtgtcattgccttgaaatgcttgcttag |
| SEQ ID | ggcttcttttatgttatcttaaaaagtgctggtgaattttccattttttacatccatttcacatgtaagagacacaaaagtct |
| NO: 406 | agattggtcttgatattgagataataaaaagtaagtagcattaagaaaggtaacaatcttcattctacagatgaactcattga |
| | aacaatttaggggaatgaggggcaaaaggggagaaatactgctaaagaacatgagcataaaaatgcgtgcgtttcagtgttta |
| | agaaggcttgataaagaatgtcacttttttatttaactgataagatttttgttattttttactttgataagtaaaccaaagaa |
| | tatttgtatttcaagcagtttgtgtggtgtttctatataattttctgtgtataaataataaagtaggcatttgtttattttgt |
| | aaaaaagaaatgaaaatctgctggccagctatgtcctctaggaaatgacagacccaaccacca |
| 209606_at | gaattgcaaaactgacatcccatttcacagcaatagtgacctttatttaaattgttgtgttatagtttatgcttcttaaatca |
| SEQ ID | tttttcaacctaaacagccaatttctaagcagacaggaaaactaaataataagttaattaatataacaaagatgcaggttcct |
| NO: 407 | gctcattccagtaatgtctttgaaagcaaaactaatatttattttctagattatccctgtgaataattgagaactttttggag |
| | tcaagtatgaataaaggtgtggcagaatataataatctggactattttctataggataattgctgggttataaaatcttaggt |
| | ttgcttatgcccagtagctcctgcggaggcttaataataggcaattttgaatttgttcaaacctgtaatggcttgtaaacaaa |
| | gatgaccatcagctgtttctcacatctatagtgacaataaagcgggaagtataagatttaataggaggggttaaggttcatga |
| | gaaccatggaaagatgtggtctgagatgggtgctgcaaagat |
| 209613_s_at | gcagatttcttgcttcatatgacaaagcctcaattactaattgtaaaaactgaactattcccagaatcatgttcaaaaaatct |
| SEQ ID | gtaatttttgctgatcgaaagtgcttcattgactaaacagtattagtttgtggctataaatgattatttagatgatgactgaa |
| NO: 408 | aatgtgtataaagtaattaaaagtaatatggtggctttaagtgtagagatgggatggcaaatgctgtgaatgcagaatgtaaa |
| | attggtaactaagaaatggcacaaacaccttaagcaatatattttcctagtagatatatatatacacatacatatatacacat |
| | atacaaatgtatatttttgcaaaattgttttcaatctagaacttttctattaactaccatgtcttaaaatcaagtctataatc |
| | ctagcattagtttaatattttgaatatgtaaacacctgtgttaatgctttgttaatgcttttcccactctcatttgttaatgc |
| | tttcccactctcgggaaggatttgcattttgagctttatctctaaatgtgacatgca |
| 209774_x_at | agagagacacagctgcagaggccacctggattgcgcctaatgtgtttgagcatcacttaggagaagtcttctatttatttatt |
| SEQ ID | tatttatttatttatttgtttgttttagaagattctatgttaatattttatgtgtaaaataaggttatgattgaatctacttg |
| NO: 409 | cacactctcccattatatttattgtttattttaggtcaaacccaagttagttcaatcctgattcatatttaatttgaagatag |
| | aaggtttgcagatattctctagtcatttgttaatatttcttcgtgatgacatatcacatgtcagccactgtgatagaggctga |
| | ggaatccaagaaaatggccagtaagatcaatgtgacggcagggaaatgtatgtgtgtctattttgtaactgtaaagatgaatg |
| | tcagttgttatttattgaaatgatttcacagtgtgtggtcaacatttctcatgttgaagctttaagaactaaaatgttctaaa |
| | tatcccttggacattttatgtctttcttgtaagatactgccttgtttaatgttaattatgcagtgtttccctc |
| 209795_at | tagtctaattgaatcccttaaactcagggagcatttataaatggcaaatgcttatgaaactaagatttgtaatatttctctct |
| SEQ ID | ttttagagaaatttgccaatttactttgttatttttccccaaaaagaatgggatgatcgtgtatttatttttttacttcctca |
| NO: 410 | gctgtagacaggtccttttcgatggtacatatttctttgcctttataatcttttatacagtgtcttacagagaaaagacataa |
| | gcaaagactatgaggaatatttgcaagacatagaatagtgttggaaaatgtgcaatatgtgatgtggcaaatctctattagga |
| | aatattctgtaatcttcagacctagaataatactagtcttataataggtttgtgactttcctaaatcaattctattacgtgca |
| | atacttcaatacttcat |
| 209894_at | ggcataggaacagttttctcttcatatattatgattatgaataataggaaagttgtattaattcagtatttgtcattatgcag |
| SEQ ID | tattttaatacctacataagtctattccattattatattttgcgcttggcatatttattcctttatgcttttaatcactaaca |
| NO: 411 | tattttacttaagagtataaaactatgctaaataaattgtattgtatatggaatatgcttgccattatgaagaacagctgggt |
| | ataccattatttcaagtaataaatctgaattctattagtttaaaaattgtaaaattcaagttaaatgacgtgtatgatatata |
| | agctgaacacattttctatagcccttaatttagttgtgttaatttttcaaggtgatgtatcaacagctttttttttttgcatt |
| | tgttttttcaatgtgtttacattgtatgaattgagctttttgcccacagattcttgatttgtagttgtttggcagga |
| 210260_s_at | gattgagtcatcgacattcaggatttaagtctgaggtagtcaaccctcaggaaaaaaaaaatggcttatctgaaatcagtact |
| SEQ ID | gtggaaatgaactatattagctattatgaataatgtccagtataagaatatgcttctggaattgagttctccttttaagtacc |
| NO: 412 | aatgatacttaaatttctcagaaatgtaatggtgtgtcattgccttgaaatgcttgcttagggcttcttttatgttatcttaa |
| | aaagtgctggtgaattttccattttttacatccatttcacatgtaagagacaaaaaagtctagattggtcttgatattgagat |
| | aataaaaagtaagtagcattaagaaaggtaacaatcttcattctacagatgaactcattgaaacaatttaggggaatgagggg |
| | caaaaggggagaaatactgctaaagaacatgagcataaaaacgcgtgcgtttcagtgtttaagaagg |
| 210538_s_at | gaaagtgtcctatttgtaggagtacaatcaagggtacagttcgtacatttctttcatgaagaagaaccaaaacatcatctaaa |
| SEQ ID | ctttagaattaatttattaaatgtattataactttaacttttatcctaatttggtttccttaaaatttttatttatttacaac |
| NO: 413 | tcaaaaaacattgttttgtgtaacatatttatatatgtatctaaaccatatgaacatatattttttagaaactaagagaatga |
| | taggcttttgttcttatgaacgaaaaagaggtagcactacaaacacaatattcaatcaaaatttcagcattattgaaattgta |
| | agtgaagtaaaacttaagatatttgagttaacctttaagaattttaaatattttggcattgtactaatacctggttttttttt |
| | tgttttgtttttttgtacagacagggcagcatactgagaccctgcctttaaaaacaaacagaacaaaaacaaaacaccaggga |
| | cacatttctctgtcttttttgatcagtgtcctatacatcgaaggtgtgcata |
| 210915_x_at | aaaggccacactggtgtgcctggccacaggtatcttccctgaccacgtggagctgagctggtgggtgaatgggaaggaggtgc |
| SEQ ID | acagtggggtcagcacggacccgcagcccctcaaggagcagcccgccctcaatgactccagatactgcctgagcagccgcctg |
| NO: 414 | agggtctcggccaccttctggcagaacccccgcaaccacttccgctgtcaagtccagttctacgggctctcggagaatgacga |
| | gtggacccaggatagggccaaacccgtcacccagatcgtcagcgccgaggcctggggtagagcagactgtggctttacctcgg |
| | tgtcctaccagcaaggggtcctgtctgccaccatcctctatgagatcctgctagggaaggccaccatgtatgctgtgctggtc |
| | agcgcccttgtgttgatggccatggtcaagagaaaggatttctgaaggcagccctggaagtggagttaggagcttctaacccg |
| | tcatggtttcaatacacattcttcttttgccagc |
| 210972_x_at | ggaacaagacttcaggtcacgctcgatatccagaaccctgaccctgccgtgtaccagctgagagactctaaatccagtgacaa |
| SEQ ID | gtctgtctgcctattcaccgattttgattctcaaacaaatgtgtcacaaagtaaggattctgatgtgtatatcacagacaaaa |
| NO: 415 | ctgtgctagacatgaggtctatggacttcaagagcaacagtgctgtggcctggagcaacaaatctgactttgcatgtgcaaac |
| | gccttcaacaacagcattattccagaagacaccttcttccccagcccagaaagttcctgtgatgtcaagctggtcgagaaaag |
| | ctttgaaacagatacgaacctaaactttcaaaacctgtcagtgattgggttccgaatcctcctcctgaaagtggccgggttta |
| | atctgctcatgacgctgcggctgtggtccagctgagatctgcaagattgtaagacagcctgtgctccct |
| 211603_s_at | ggtgctgccctgtgtacatataaatgaatctggtgttggggaaaccttcatctgaaacccacagatgtctctggggcagatcc |
| SEQ ID | ccactgtcctaccagttgccctagcccagactctgagctgctcaccggagtcattgggaaggaaaagtggagaaatggcaagt |
| NO: 416 | ctagagtctcagaaactcccctgggggtttcacctgggccctggaggaattcagctcagcttcttcctaggtccaagcccccc |
| | acaccttttccccaaccacagagaacaagagtttgttctgttctgggggacagagaaggcgcttcccaacttcatactggcag |
| | gagggtgaggaggttcactgagctccccagatctcccactgcggggagacagaagcctg |
| 211700_s_at | ccagcacgggcttcagtagtggacccagttctattgttggcttcagcggtggaccaagcactggtgttggcttctgcagtgga |
| SEQ ID | ccaagcaccagtggcttcagcggtggaccgagcacaggagctggcttcggcggtggaccaaacactggtgctggctttggtgg |
| NO: 417 | tggaccgagcaccagtgctggctttggcagtggagccgccagtcttggtgcctgtggcttctcgtatggctagtgagg |
| 211796_s_at | gccatcagaagcagagatctcccacacccaaaaggccacactggtgtgcctggccacaggtttctaccccgaccacgtggagc |
| SEQ ID | tgagctggtgggtgaatgggaaggaggtgcacagtggggtcagcacagacccgcagcccctcaaggagcagcccgccctcaat |
| NO: 418 | gactccagatactgcctgagcagccgcctgagggtctcggccaccttctggcagaacccccgcaaccacttccgctgtcaagt |
| | ccagttctacgggctctcggagaatgacgagtggacccaggatagggccaaacctgtcacccagatcgtcagcgccgaggcct |
| | ggggtagagcagactgtggcttcacctccgagtcttaccagcaaggggtcctgtctgccaccatcctctatgagatcttgcta |
| | gggaaggccaccttgtatgctgtgctggtcagtgccctcgtgctgatggccatggtcaagagaaagga |
| 212587_s_at | ggagccaatccatgcagatattttgttggaaacttataagaggaagattgctgatgaaggaagactttttctggctgaatttc |
| SEQ ID | agagcatcccgcgggtgttcagcaagtttcctataaaggaagctcgaaagccctttaaccagaataaaaaccgttatgttgac |
| NO: 419 | attcttccttatgattataaccgtgttgaactctctgagataaacggagatgcagggtcaaactacataaatgccagctatat |
| | tgatggtttcaaagaacccaggaaatacattgctgcacaaggtcccagggatgaaactgttgatgatttctggaggatgattt |
| | gggaacagaaagccacagttattgtcatggtcactcgatgtgaagaaggaaacaggaacaagtgtgcagaatactggccgtca |
| | atggaagagggcactcgggcttttggagatgt |
| 212588_at | tcctccttgttctactcatatatatctatcttatatagtttactattttacttctagagatagtacataaaggtggtatgtgt |
| SEQ ID | gtgtatgctactacaaaaaagttgttaactaaattaacattgggaaatcttatattccatatattagcatttagtccaatgtc |
| NO: 420 | tttttaagcttatttaattaaaaaatttccagtgagcttatcatgctgtctttacatggggttttcaattttgcatgctcgat |
| | tattccctgtacaatatttaaaatttattgcttgatacttttgacaacaaattaggttttgtacaattgaacttaaataaatg |
| | tcattaaaataaataaatgcaatatgtattaatattcattgtataaaaatagaagaatacaaacatatttgttaaatatttac |
| | atatgaaatttaatatagctatttttatggaatttttcattgatatgaaaaatatgatattgcatatgcatagttcccatgtt |
| 212592_at | gtatcaaaatcttccaattatcatgctcacctgaaagaggtatgctctcttaggaatacagtttctagcattaaacaaataaa |
| SEQ ID | caaggggagaaaataaaactcaaggagtgaaaatcaggaggtgtaataaaatgttcctcgcattcccccccgcnttttttttt |
| NO: 421 | ttttttgactttgccttggagagccagagcttccgcattttctttactattctttttaaaaaaagtttcactgtgtagagaac |
| | atatatgcataaacataggtcaattatatgtctccattagaaaaataataattggaaaacatgttctagaactagttacaaaa |
| | ataatttaaggtgaaatctctaatatttataaaagtagcaaaataaatgcataattaaaatatatttggacataacagactt |
| 212654_at | ggtggccgagagtaaatgtggggacctagaggaggagctgaaaattgttaccaacaacttgaaatccctggaggcccaggcgg |
| SEQ ID | acaagtattccaccaaagaagataaatatgaagaggagatcaaactgttggaggagaagctgaaggaggctgagacccgagca |
| NO: 422 | gagtttgccgagaggtctgtggcaaagttggagaaaaccatcgatgacctagaagatgaagtctatgcccagaagatgaagta |
| | caaggccattagcgaggaactggacaacgcactcaatgacatcacctccctctgagccccacgccagcgtggccacctcagct |
| | ctcttctctcctctcctttccattctctctatggggaggggagcaggcaggaggagcagaaattgccaacattgcacagccag |
| | gctgggagcagcctagggagagcccc |
| 212671_s_at | accaatgaggttcctgaggtcacagtgttttccaagtctcccgtgacactgggtcagcccaacaccctcatctgtcttgtgga |
| SEQ ID | caacatctttcctcctgtggtcaacatcacntggctgagcaatgggcactcagtcacagaaggtgtttctgagaccagcttcc |
| NO: 423 | tctccaagagtgatcattccttcttcaagatcagttacctcaccttcctcccttctgntgatgagatttatgactgcaaggtg |
| | gagcactggggcctggatgagcctcttctgaaacactgggagcctg |
| 212813_at | ggatgcttagcatgcaagttccctccatcattgccaccttggtagagagggatggctccccaccctcagcgttggggattcac |
| SEQ ID | gctccagcctccttcttggttgtcatagtgatagggtagccttattgccccctcttcttataccctaaaaccttctacactag |
| NO: 424 | tgccatgggaaccaggtctgaaaaagtagagagaagtgaaagtagagtctgggaagtagctgcctataactgagactagacgg |
| | aaaaggaatactcgtgtattttaagatatgaatgtgactcaagactcgaggccgatacgaggctgtgattctgcctttggatg |
| | gatgttgctgtacacagatgctacagacttgtactaacacaccgta |
| 213193_x_at | tgactccagatactgcctgagcagccgcctgagggtctcggccaccttctggcagaacccccgcaaccacttccgctgtcaag |
| SEQ ID | tccagttctacgggctctcggagaatgacgagtggacccaggatagggccaaacccgtcacccagatcgtcagcgccgaggcc |
| NO: 425 | tggggtagagcagactgtggctttacctcggtgtcctaccagcaaggggtcctgtctgccaccatcctctatgagatcctgct |
| | agggaaggccaccctgtatgctgtgctggtcagcncccttgtgttgatggccatggtcaagagaaaggatttctgaaggcagc |
| | cctggaagtggagttaggagcttctaacccgtcatggtttcaatacacattcttcttttgccagcgcttctgaagagctgctc |
| | tcacctctctgcatcccaatagatatccccctatgtgcatgcacacctgcacactcacggctgaaatctccctaacccagggg |
| | gaccttagcatgcctaagtga |
| 213539_at | gggaacactgctctcagacattacaagactggacctgggaaaacgcatcctggacccacgaggaatatataggtgtaatggga |
| SEQ ID | cagatatatacaaggacaaagaatctaccgtgcaagttcattatcgaatgtgccagagctgtgtggagctggatccagccacc |
| NO: 426 | gtggctggcatcattgtcactgatgtcattgccactctgctccttgctttgggagtcttctgctttgctggacatgagactgg |
| | aaggctgtctggggctgccgacacacaagctctgttgaggaatgaccaggtctatcagcccctccgagatcgagatgatgctc |
| | agtacagccaccttggaggaaactgggctcggaacaagtgaacctgagactggtggcttctagaagcagccattaccaactgt |
| | acct |
| 213790_at | gtttgagttttgtagggccatcacctggaaagtcaatgtgactagacacaaagtagcccagaggctacttttcttcctacagc |
| SEQ ID | ttattatagttgtaggttctatgacctcacttcatgggttccaggcaattccgctgaaaggtttgtctcctgaaattttttaa |
| NO: 427 | gtttgttttcctgacacatgtaatcagatgtgtagcaaccgagggaaacgaagcctaacattctccattgtggaaatacacac |
| | aggaggttacanttcacagcgtggatttttccagcttacacatgtgggatgacatcacagaaaccacaaaagcagcaaattaa |
| | actgtaggagagtcaatactcctgacgagtctcnggggggggcatttttatgccttcttaactttatgagaattctcaggctg |
| | aactataggccattgttccc |
| 214470_at | ggttcaccttggcatcaatttgccctgaaacttagctgtgctgggattattctccttgtcttggttgttactgggttgagtgt |
| SEQ ID | ttcagtgacatccttaatacagaaatcatcaatagaaaaatgcagtgtggacattcaacagagcaggaataaaacaacagaga |
| NO: 428 | gaccgggtctcttaaactgcccaatatattggcagcaactccgagagaaatgcttgttattttctcacactgtcaacccttgg |
| | aataacagtctagctgattgttccaccaaagaatccagcctgctgcttattcgagataaggatgaattgatacacacacagaa |
| | cctgatacgtgacaaagcaattctgttttggattggattaaatttttcattatcagaaaagaactggaagtgganaaacggct |
| | cttttttaaattctaatgacttagaaattagaggtgatgctaaagaaaacagctgtatttccatctcaca |
| 215313_x_at | tgagatgggagctgtcttcccagcccaccatccccatcgtgggcatcattgctggcctggttcnccttggagctgtgatcact |
| SEQ ID | ggagctgtggtcgctgccgtgatgtggaggaggaagagctcaggtggagaaggggtgaaagatagaaaaggagggagttacac |
| NO: 429 | tcaggctgcaagcagtgacagtgcccagggctctgatgtgtctctcacagcttgtaaagtgtgagacagctgccttgtgtggg |
| | actgagaggcaagagttgttcctgcccttccnnttgtgacttgaagaaccctgactttgtttctgcaaaggcncctgcatgtg |
| | tctgtgttcgtgtaggcataatgtgaggaggtggggagaccaccccaccccgatgtccaccatgaccctcttcccacgctgac |
| | ctgtgctccctctccaatcatctttcctgttccagagaggtggggctgaggtgtctccatctctgtctcaacttcatggtgca |
| | ctgagctgtaacttcttc |
| 216438_s_at | acagagacgcaagagaaaaatccactgccttccaaagaaacgattgaacaggagaagcaagcaggcgaatcgtaatgaggcgt |
| SEQ ID | gtgccgtcaatatgcactgtacattccacaagcattgccttcttattttacttcttttagctgtttaactctgtaagatgcaa |
| NO: 430 | agaggttggatcaagtttaaatgactgtgctgcccctttcacatcaaagaactactgacaacgaaggccgcgactgcctctcc |
| | catctgtctatctggctggcagggaaggaaagaacttgcatgttggtgaaggaagaagtggggtgggacaacagtgaaatcta |
| | aagtaaaaccaagctggcccaaggtgtcctgcaggctgtaatgcagtttaatcagagtg |
| 216920_s_at | cactactgctgcagctcacaaacacctctgcatattacatgtacctcctcctgctcctcaagagtgtggtctattttgccatc |
| SEQ ID | atcacctgctgtctgcttngaagaacggctttctgctgcaatggagagaaatcataacagacggtggcacaaggaggccatct |
| NO: 431 | tttcctcatcggttattgtccctagaagcgtcnncnnannnnnnnnttgggctttctttctgggtttgggccatttcagttct |
| | catgtgtgtactattctatctattgtataatggttttcaaaccagtgggcacacagagaacctcactctgtaataacaatgag |
| | gaatagccatggcgatctccagcaccaatctctccatgttttccacagctcctccagccaacccaaatagcgcctgctatagt |
| | gtagacagcctgcggcttctagccttgtccctctcttagtgttctttaatcagataactgcctggaagcctttcattttacac |
| | gccctgaagcagtcttctttgctagttgaattatgtggtgtgtttttccgtaata |
| 217767_at | ggtctacgcctattacaacctggaggaaagctgtacccggttctaccatccggaaaaggaggatggaaagctgaacaagctct |
| SEQ ID | gccgtgatgaactgtgccgctgtgctgaggagaattgcttcatacaaaagtcggatgacaaggtcaccctggaagaacggctg |
| NO: 432 | gacaaggcctgtgagccaggagtggactatgtgtacaagacccgactggtcaaggttcagctgtccaatgactttgacgagta |
| | catcatggccattgagcagaccatcaagtcaggctcggatgaggtgcaggttggacagcagcgcacgttcatcagccccatca |
| | agtgcagagaagccctgaagctggaggagaagaaacactacctcatgtggggtctctcctccgatttctggggagagaagccc |
| | aacctcagctacatcatcgggaaggacacttgggtggagcactggcctgaggaggacgaatgccaagacgaagagaaccagaa |
| | acaatgccaggacctcggcgccttcaccgagagcatggttgtc |
| 218499_at | aatgtttaattgtttggatctgcacagtttggtttttgcacaaaagtcatttaaaaaaatctgagtaattgtcaaatattaaa |
| SEQ ID | agaaagatattcttcctgtaaggaatacagtttttagtcaaagtggccattacatcctctttttaatttacataatacagata |
| NO: 433 | cttgagaaagttgttgtggtgttgtatgccaagaaaattctttttattggtgcctatattgtaacaattatttttaatgcatt |
| | gtattttgaagtaacggttcagttaaatttttcacctgctgtgtaactgaagcacaattacagtttataatcatctgtagaag |
| | tctggagataattttgcaactcatgttatgggttaaatgaatatttttgtaaaagtaaaagcaacaaatttataaattgatta |
| | tttgaaactttacaacacaattgcatcccaaatacaaattgtattgcttattcattatagctattcgtcctgtaatctgtttc |
| | taggtgaagcatactccagtgttttaggggttt |
| 218805_at | taaatctctggaccctggagcacttctaatgtatcaccccatggagtcattgttctaataatcaccaattcagactcagatcc |
| SEQ ID | tcgtggtctatggagcatgctgcttgctgtctgtgcagctcccatttccccttcttcctgatagacttggagctgtgtgcctc |
| NO: 434 | cactccaaggctgcctgcctgctgtaaacactattccactctgtctgccaacaactgcttcaggaatgggcctgagatcccat |
| | gcaggtccctgagaagtgagtaaaagtccgcagaggtggggatggaagatctctccttagatagaacctgtcttcctccctgg |
| | cattgtggggtctgggcgtgacactgggactctcagcagctttgtgctgccaacctgagattgaaggcagtgcctcagagcag |
| | cacagagagttggggccccctgagccctgagccaccagccctgcagcctgccctatctccgcatttccagttgtatta |
| 218807_at | tgccgtcagccgaactttgttatggagggagcagcctcacacaagcagaaacactcctgtggatggtattgtagcatgtattg |
| SEQ ID | tttattttagtcaatagaccctctccttataaatggtgtttagtcttcctgttgcatttcatgggcctgggggtttcctngca |
| NO: 435 | gaggatattggagcccctttttgtgacattaccaattacatctttgtccacgtttaatactttgttttggaaaatttaaatgc |
| | tgcagatttgtgtagagttctaataccaaagacagaagtaaatgttttccatatactttgtcttgcctgtatgcagcccttgt |
| | gtaatatggt |
| 222838_at | aacacctgtgctaggtcagtctggcacgtaagatgaacatccctaccaacacagagctcaccatctcttatacttaagtgaaa |
| SEQ ID | aacatggggaaggggaaaggggaatggctgcttttgatatgttccctgacacatatcttgaatggagacctccctaccaagtg |
| NO: 436 | atgaaagtgttgaaaaacttaataacaaatgcttgttgggcaagaatgggattgaggattatcttctctcagaaaggcattgt |
| | gaaggaattgagccagatctctctccctactgcaaaaccctattgtagta |
| 222943_at | gaagagaaaaccactaaccttgatttttgtattgcaaaatcagatagacctggaaacataaatttaaatccttagacattttt |
| SEQ ID | ctagaaaaaaatgcaaagtttataaagatgatacaaccatgatttgcaactgtaacaggagaccatttattataagcgtacct |
| NO: 437 | gtttgtgaacttaattattctgattccataagctgtttttgcttaggtgatccactgccatgtgatccataatttttctacat |
| | aaaaaatcaaagttaaaagtcacattatacagttatgcattcatttcaacaaaatagtgaattgataatctacttgttaatat |
| | attcggcccatattttgtgtgtttggacaagtacatctcccttttgcctaatgaactttt |
| 223403_s_at | cattaccaggttactcttgagatttttcaatggtgttagaactctcaaccaagacctgaaaaccaagtatgcaaggtttctga |
| SEQ ID | atctctctggtagattaactattgacaatgattttctgttatctttgttcaaaaagttcatgtcttctcaaaatatgaaatat |
| NO: 438 | tgataaatggaagagcatacggtgacaagtctcctttccaaccccaggttccctacaccctgctctcagcaggcagtgagtgt |
| | cacacacctgttaatccatcttgagcaggacagtactatacaaatagaatgcaagctgtaatgtaattttatattttcttata |
| | gccacgttgaagtaaaaacaaacaggtacagtgttttttaccagctttatagaagtacagttgttacatatttaatgaataca |
| | atttgatgggtctgactatatgcacacacctttgataccatcac |
| 223593_at | ggcagctgcagacaagtggttaactggtttggcagaatggcatgttcctgctgctggaatgtttttatggattaaagttaaag |
| SEQ ID | gcattaatgatgtaaaagaactgattgaagaaaaggccgttaagatgggggtattaatgctccctggaaatgctttctacgtc |
| NO: 439 | gatagctcagctcctagcccttacttgagagcatccttctcttcagcttctccagaacagatggatgtggccttccaggtatt |
| | agcacaacttataaaagaatctttatgaagaaattaaactaggttgggcatggtgcgtcacacctataatcccagcactttgg |
| | gaggcagaggagggaggatcacttgaacccaggaattcaggctgcagtaagctacgatcacaccactgcactctggcctgcat |
| | gcactctggcctgcatggcagaacaagaccctgtctctaaaaaaagagaaagaaatcaaactaatcatgctgctcat |
| 224896_s_at | ttccccaaaactcaactcctatggcaattatgaactccattttaccaagaacatttaagtgcctcagcatctgtatgatatag |
| SEQ ID | tggagcaggtgctgacataggtaccagctgacatgatgtgtcactagctctgtgggatgattgccacatacatggaacacctg |
| NO: 440 | ggagtgctggaaatgtactgggatcgaagtgacaaagtgtgttttcattcacagtggaggctacatcaagcaaggggaggtcc |
| | agccctcttgcaagtgtggtgagaggctctactagcaaagacatgggcaccggagtaggtcccgtgtagcatgcgggtgctgt |
| | agagaaaattcagtgacgtacatggctctggttctggacacaaaatctgtactg |
| 224908_s_at | accaagcctggctttatgtatttatttctgttcatgcggaatgattggttcagaactgttcctttcccttccatgatgtcctt |
| SEQ ID | gacacagaaggttatgcctggctcccagtcaggcttcatacttttggtccatgtaagtgctacccgttgctgggggaggagtc |
| NO: 441 | atggtttatttggaaatgtcagttgcaatcatggttctgtcatttgactgcacagtatcagaggagcctgttaacctctctgt |
| | gccttagtttcttagcccatgaaagagatcattgcctgacccagggactacctcaagggcttttgatgaggacaagtgacagt |
| | aggaagatgcaagagcctttagtaccaaggttctcaacactgactacatgctggaatgactgtgaagcttttaaaaaatgtta |
| | gtgcccactcttcccctgtacccccggacagttaaatcagaacctcagacagcaatatgccttgagatgccttgaaccatgct |
| | tgaga |
| 225502_at | gaagccttactacaattccaaaaatcatcatggttggaaatttgggaggagattatttgtgaacttgttacccttttggtaat |
| SEQ ID | ggtggactaattgctgtatagttatttttgttttattattactgttacattaatttaacatgcatttatagaagaatacattc |
| NO: 442 | aaagcactgatgtaggagatacacggtacttggagcagtcagccaaaaatcacagatactgctttcacttaaatggaaacaat |
| | tctccgataatgctttgctttttttcttatgtcactcttgtgtactatctatttttctcctctctgggaccaagtttcttttt |
| | ataaagcaataatatctctgttttcatttcagaacatt |
| 225882_at | gcaccttacatatttgatgctcaggaagaaagtgcaagaagatccctaatagaacattaaagattcttaaaagtttttgaagt |
| SEQ ID | aggctgcttggggtgaccagccccacccttacatctccatagttggtacagttagcttgtagcagctgaagctgatgcctgga |
| NO: 443 | gatcactgtctgttggtctgatctcagtatcatactgagacacctcccctgagccttacctacttaaattggtaaccgtccta |
| | ccaaaatttgtcaattttaatcaagtgaggcaagttgcaagggagccagagatacgtgaaaaaagaaaagcagaaatactgat |
| | actttctaagaaagaagttgtaataatttctttggcacattgacttactgatatcttttgaaatgcatagangactcttgtga |
| | accaaagagagcagtagtggttattccccggcgcaataaaaatgcc |
| 226218_at | ctaattggttctgcccaatctcctttcagattttattaggaaaaaaaaataaacctcctgatcggagacaatgtattaatcag |
| SEQ ID | aagtgtaaactgccagttctatatagcatgaaatgaaaagacagctaatttggtccaacaaacatgactgggtctagggcacc |
| NO: 444 | caggctgattcagctgatttcctaccagcctttgcctcttccttcaatgtggtttccatgggaatttgcttcagaaaagccaa |
| | gtatgggctgttcagaggtgcacacctgcattttctnagctcttctagaggggctaagagacttggtacgggccaggaagaat |
| | atgtggcagagctcctggaaatgatgcagattaggtggcatttttgtcagctctgtggtttattgttgggactattctttaaa |
| | atatccattgttcactacagtgaagatctctgatttnaccgtgtactatccacatgcattacaaacatttcgcagagctgct |
| 226219_at | gtctccgacacaaagcacttatctcttaggagattcccaagaaagtcaacaagatcttgttcccagggagtgggtcattggcc |
| SEQ ID | aaagggaacataaggtaggcagaaaacttaaaagagtttgttaaagtgaagactggagaaattcctcccttcctctgagctgt |
| NO: 445 | gaatctctcttcatgaaagccaaaggtagagacagggaggacagggccaggttagggccttccacacacaaacacttctagag |
| | ttgcccattcctgttatgttcttggaccctaagatacctcctgtcccttttaaatccagattaagagaaacgtccaggaagag |
| | ctctttgaagccctcaatatttgttggagggactggactcctctccagctccccaccctctgcctccagtcaccatgtgcaag |
| | agaggtcctgtacagatctctctgggctctcctttctcctttggaataacttgttcctatttcaggaaagggaaatggtgtca |
| | ctcaggccctgggactgcttctccagccaggctggggccacaggtcccactctagtgaaggt |
| 227265_at | acatctgctagaaccttttgccttaactattcaccaatatatgctaatattcataaatatggattgactgtttacaaacatta |
| SEQ ID | gaatcttgtcttggttccattttgatggctaatatttgttatcttaattaagactatttctgaggtcatgattacttgaaaat |
| NO: 446 | attgactaaaactgggtccttagaaattccaggtggagctgatttacctatgactgaggggaaaaaaaaatcaaattttactg |
| | ataatagtaatgctccaaatgaattaatgacacatctgttcaataaataaagagcttaaatatacaaaacataagaaatctgg |
| | gcaacaaaacttgtggtctttacttttgaatagctacccaagaaaaggttttaaaggtaaaagttatgagtaatgtcatcaca |
| | ataagctcttgtttaaaattcttttcttttatgtataattaggtttatgtttcatgtcttt |
| 227346_at | tgcttttggtgcgatggcactcactgtgaacatgtgtaaccacatattaatatgcaatattgtttccaatactttctaataca |
| SEQ ID | gttttttataatgttgngtgtggtgattgttcaggtcgaatctgttgtatccagtacagctttaggtcttcagctgcccttct |
| NO: 447 | ggcgagtacatgcacaggattgtaaatgagaaatgcagtcatatttccagtctgcctctatgatgatgttaaattattgctgt |
| | ttagctgtgaacaagggatgtaccactggaggaatagagtatccttttgtacacattttgaaatgcttcttctgtagtgatag |
| | aac |
| 227361_at | ttcccaggattctctttgggggtcattttgtgtgacagatatattttagacatttggagaaacagtttcagatcctgccagga |
| SEQ ID | tatttttgtaaaaaaggaaaatggaagattccaataaactagaaacagtacgtatctaagatgctgacacagaagctaatgtg |
| NO: 448 | acttttcagcttatcaagaggatggccaataaaacttaaaggtgtggttagatgttttctcacttttgtgacattaatttatc |
| | actgagtctcattcaaccaagtaatctaaaatactgtgcaaattctagcagtatgtcttcgataacttggatgttaggatagc |
| | caatatgtacaaaaaattaaatcaagtattttgtcctatgtataacacaaattaattttacacagagaaagatgtttctaggc |
| | aagtgaaattctggtaattcatactatttct |
| 227550_at | tgttgcatttttcactcttaacccgagggtgtgtttcagcttatgttcgttctgtttcatgcaggtttatagcacggtagagt |
| SEQ ID | agaaggcggcttctgatttttagggtatttttagaattcattcctgagtgaggggttcagacacccagtctcctcggaacagg |
| NO: 449 | ggtgaggggtcgactgagctttgttgagaagcctccagttaggcttcgggcgggtctccatgttatattgtgtgtttactgag |
| | cttcccactggtagaagatgacacatttgtccatcgtcctgtgtatctgctttccagaggacaccggagcattctcctggggt |
| | cactcccacatggctgcctcacatagctgttttgcaacagcctttattgccaacacccttggaagaacatctcctgtagaagt |
| | acaagttttgcaatatggactttggaagtggttttgtgatgttcgaattttctgttagcctattgctcaagcactacagaatg |
| | tacatagtccctttggcca |
| 228017_s_at | gcctgcgtaagtgaggaaacagctgatcctgctcctgtggcctccagcctcagcgaccgaccagtgacaatgacaggagctcc |
| SEQ ID | caggccttgggacgcgcccccacccagcaccccccaggcggccggcagcacctgccctgggttctaagtactggacaccagcc |
| NO: 450 | agggcggcagggcagtgccacggctggctgcagcgtcaagagagtttgtaa |
| 228071_at | aggagttggtggagctgatagagaaaatggtgcagtgcaacgaaggggcttacttttctgatgacatatacaaggacacagag |
| SEQ ID | gaaaggctgaaacaacgggaagaggttttgaggaaaatctacactgaccaattaaatgaagaaattaaactagtagaagagga |
| NO: 451 | taagcataaatcagaggaagaaaaggagaaagaaattaaattactaaaattaaaatatgatgaaaaaataaaaaatataaggg |
| | aagaagctgagagaaatatatttaaagatgtttttaataggatttggaagatgctttcagaaatatggcataggtttttgtcg |
| | aaatgtaagttttattcttcctaatttactgtgatttgttaatggatgaattgtattttgcaaagatagttagagaaatacct |
| | ccttcc |
| 228094_at | gggagaaacacatttactccccaataattgtacgggaggtgatcgaggaagaagaaccaagtgaaaaatcagaggccanctac |
| SEQ ID | atgaccatgcacccagtttggccttctctgaggtcagatcggaacaactcacttgaaaaaaagtcaggtgggggaatgccaaa |
| NO: 452 | aacacagcaagccttttgagaagaatggagagtcccttcatctcagcagcggtggagactctctcctgtgtgtgtcctgggcc |
| | actctaccagtgatttcagactcccgctctcccagctgtcctcctgtctcattgtttggtcaatacactgaagatggagaatt |
| | tggagcctggcagagagactggacagctctggaggaacaggcctgctgaggggaggggagcatggacttggcctctggagtgg |
| | gacactggccctgggaaccaggctgagctgagtggcctcaaaccccccgttggatcagaccctcctgtgggcagggttcttag |
| | tggat |
| 228786_at | cacacgcagatcctgcactgtactgtccagtcaactgacttcagcaaagatgtctgggttcagcagcgagttgctgtgtgagg |
| SEQ ID | ccaccagaatggaggtgatatcggcctcagtgctcatcctggaggtggagaagtggtcagagtcctctgtggtgaaatggccc |
| NO: 453 | tacaccaaagtgggagacattcagaatcgaggcgaaattggcctcagtgctcctcttggaggcagagaagcagtaggagtcgg |
| | tggtgaaatggccctgtgtgaatgtggaagaccagcagattggaggtggaattggcctcagtgcttgtcctggaggtggagaa |
| | gcggtcagagtccttggtggtgaagtggccctgcacaaaacgttgctcccccttggccggtgaaagaataaaagaaagctggc |
| | cncttgcatttcngtttcagttnanagaanacatgttttggaaatagtatcttctagatctgtctttttctcttctgatttaa |
| | cttagtaaagctaagttttatctcttc |
| 228812_at | gagacttcattggtgatacactcaatttttactgggtaattagctaataatgttggtcactgtctcacagttcaagtagcttt |
| SEQ ID | aagatgatgtggcaaggaaaacacaaagcttttgggtaaccagcgttcttaaatgtatggtttttgaccaggtgaacccttta |
| NO: 454 | gaagtgatttctgttttaaaagtatgtacttaaaatacctttggctgtgatgaatgtagatcccagcagaataccaaaatcct |
| | attttttttgactgagtatttgtagatgcttaatgactgaaatgaatttggaggcactgatgaaagtgatttttttnaagttc |
| | tcaggtactgttcaattat |
| 229152_at | tgaagaaagttctcctcctgatcacagccatcttggcagtggctgttggtttcccagtctctcaagaccaggaacgagaaaaa |
| SEQ ID | agaagtatcagtgacagcgatgaattagcttcagggttttttgtgttcccttacccatatccatttcgcccacttccaccaat |
| NO: 455 | tccatttccaagatttccatggtttagacgtaattttcctattccaatacctgaatctgcccctacaactccccttcctagcg |
| | aaaagtaaacaagaaggaaaagtcacgataaacctggtcacctgaaattgaaattgagccacttccttgaagaatcaaaattc |
| | ctgttaataaaagaaaaacaaatgtaattgaaatagcacacagcattctctagtcaatatctttagtgatcttctttaata |
| 229598_at | atttttcttagctaaatctggcaactgtgnctatttcattgaaaacctgaaagtgtacaaagaaggaagaagcagaatctgcc |
| SEQ ID | atatgagtaatagaagtgagcaggcccaggactccctaagtcaagaaaccaagaggcgtcattacggaaaagagtaactcacc |
| NO: 456 | ctgtgtgctccttggtagttctccctcagcgatgcccccatgttatgaatggggaaaagttcactgaagggttcatagtgaag |
| | aaactttttggatgatttctgttggtgggttttggataccttcaagggatcagaaaataatatacttaggaaattttggtaat |
| | gtcatcattactctctacattattattatgacggttacaattgttaaatctaggtggtgggtatgtgggttatattgtacatg |
| | atttttaacttgtctgcatgtttgaaa |
| 231229_at | gcacgtccaaggtgatcctgagggctgtggcggacnaaggggacctgcaagtatntgtccctgnncaccctgaagaaggctgt |
| SEQ ID | ttccaccacgggntacgacatggcccgaaatgcctatcacttcaagcgtgtgctcaaggggctggtggacaagggctcagcag |
| NO: 457 | gtgaccggcangggggcctcaggctccttcaccctgggcaagaagcaggcctccaagtccaagctcaaggtcaagaggcaacg |
| | acagcagaggtggcgctctgggcagcgcccctttggacagcacaggtcactactgggctccaaacaggggcacaagcggctta |
| | tcaagggggttcgaagggtggccaagtgccactgcaattaatgaggcaggccaggcaagcagtcaggggtgccaagancgcca |
| | ttggctcagtgcagtgggaa |
| 231929_at | ggctcaaataccatacctcagaaaatgaggtttactatggaaatactgaaacagtctttgcagctgtgtgacaagtcactcta |
| SEQ ID | ctacatactgatttggagacctccgctaaatagttttatcactgcagactaaaatgtgggacttgtatcttctttgtttttaa |
| NO: 458 | tgcacacacatacatgttctgtgcatgtatgtggttactgngtatatgtgtatgagtgttgtatatgcatgtgtgagtgtgtg |
| | tctgtatgtgtgtacaactaaagaagctgcagaaactttgtaatactttgtgaaaaggattatattataaaggtttgtactgt |
| | ctgagtgcacagctactggaataaatttagggaatctcaggaacaagcatanaannnntccaagatttatttcttctcagaag |
| | tgtaagtgcagtttttaattctgtat |
| 232311_at | acataccttgggttgatccacttaggaacctcagataataacatctgccacgtatagagcaattgctatgtcccaggcactct |
| SEQ ID | actagacacttcatacagtttagaaaatcagatgggtgtagatcaaggcaggagcaggaaccaaaaagaaaggcataaacata |
| NO: 459 | agaaaaaaaatggaaggggtggnaaacagagtacaataacatgagtaatttgatgggggctattatgaactgagaaatgaact |
| | ttgaaaagtatcttggggccaaatcatgtagactcttgagtgatgtgttaaggaatgctatgagtgctgagagggcatcagaa |
| | gtccttgagagcctcc |
| 232843_s_at | caagctaaaagagaacctcaggccaatgatcgagcggaaaattccagaactgtacaagccaatattcagagttgagagtcaaa |
| SEQ ID | agagaacattgtgctgtctgtcagcatatgtatatcagctacaaaatatattcaactttgacttcttttgacaaaggacttta |
| NO: 460 | ggaaaaagaggaacaaagacattatttgagaattaaattatatatttttaatatgactgtgaccttgactgataat |
| 235276_at | accctgcactcccaaagattttgtgcagatgggtagttccnttttttaaaaattgtgcagatatggaaaattgtgacttactt |
| SEQ ID | catgaccagaactatctagaatatgtgtgggggtataaacatcttgcttaaccaaatatctatgtaggcagaggtaaccagga |
| NO: 461 | gagaagcaagacttgctgcctaaaggagcccaccattttacttttcacatttaatctgccacgttgaatcaattggaataaaa |
| | cctgactcgcaggtgactggacaggaaatcccaaagttccaccatttctatgctta |
| 235391_at | atttcacggcaaagctttagaggtctacactgctgcctaccagaatatacaaaacattgatgaagatgaagatttagaggttt |
| SEQ ID | tccnaaattctctgtatgcaccagattattcatctcgtttagatattgtaagagcaaattcaaagtcacctcttcagagatca |
| NO: 462 | ctgtcagctaagtgtgtatctggaacaggacaggtatccacttgtcgactaagaaaggatcaacaagcagaagatgatgagga |
| | tgacgagttagatgttacagaagaagaaaattttcttaagtaaactacacatttccattttcatcataaatgacttgaaatcc |
| | acaatgactaaattgtagaactttatactcactttgctatgttaagcctcaaagtgaagtccaactgg |
| 235421_at | ataccttcacaatctgaactttctctagatgggcacagatcatgaataactcagaaaccattagcatattgtgtgcttaatta |
| SEQ ID | aggctggaaagtgctgaggctttttgcaggcgcagttagcaggcgaactggtcagcagggtttctggggaacgtagatttgcg |
| NO: 463 | tgacaggggccctgtgttgctaaggaattaatgcaggcactgagaaaagtctcagcgtcagacactcctccacctcccgggtc |
| | ctgcttccctccctctcccctttccaggcaactcacagtttaggggtgtggttgtgtgtgttcaagctcttgggttttacata |
| | taaatgcctgagacttttctgcagggtttaaaaactaacatcttgtcttacagctctttta |
| 235688_s_at | tgaggcagtgcatgttcttggcccagagtaagtgcttagtgaatgctttctaactccgaaccccagccacatccagggactgg |
| SEQ ID | gtgttgagcaaaaggggccttcaagatgttcaaggcacttggattttctcctgtctctcatcggcttttcttaacgggcctca |
| NO: 464 | gtgggtgcatgtgattatccacgtttcacctatgaaacatgaacagaggagactgacttatcagtgattcttccgcgggttcg |
| | gacagggcctcgattctgttttaaactccagtagtccctagaaattgtagctccctctagttgtggcaataggtgtgggtcct |
| | tgtgcttgcttttggcaagtttntgagctacacagggcctccattaccgtcactggtgaaatgcggctcacctccc |
| 236203_at | atgtcaggtttgtacctaccacatttaaaatagggacttgaagaattaaacattttattacaaatgaagcacttcatgcacag |
| SEQ ID | actggcacatagtaagtagtcgataggtgttaacaatttgtgttattgttattttctggagtccaactaacaaatcccacagt |
| NO: 465 | gaatgacatcacagggatgcaaccaacaagatccagaatatggaaacttctactagataaacaactccatttcttcagcaaca |
| | attcaagagagagagagaagagaagctatacattttaaaaggctgaagaaatatatgaaccaaatttgtatgaggcaatcaga |
| | aaaactgacaccgactgtattaaggaattatctaattttagtgtggtaatgagattgctgttatgttttctaa |
| 236627_at | aactagatttttgaccctcactcatctggctttccttgctatagacatgctttgcatacccagcttctagccacatcttccct |
| SEQ ID | aagaaaatttcancctggaatcagttccaaaagaaaatcttaagagatagagtatggatctgcatgttgtcagctacctaaag |
| NO: 466 | cagcaaaaattcctagatgagaataaaatcagggaaccttgcagtttaaggcacaccttaaatattaaatttttaatccttca |
| | atgcatccagccaaaacaagaaattgcaaatagctaccatccatcaatgcaaggttttt |
| 236681_at | gaaactcatgtgtcctcatggatcgtggatgccttcatttcttgagctctcaatgcagacatttaaatggctgcaatcagtag |
| SEQ ID | agtgacccgcggatggcataaatgcacctccttttcttggccttggatctatgggtctgggattgtggtcatctcctcaatcc |
| NO: 467 | tcaaaaagaggctgaatcaatgtggccgtgggtgggaacttacatacagaacccaatgaagaacttgactgtctaaacaaggg |
| | ggcctcgcatggagctgtaaagcatctaacaaatatgaaaaatgtgaagttccaaggtccaagaagaaaaataatgatgtttc |
| | tgaaagtgatgataaataattacttttaaagtgctgcatatttatacaattgagagattatttttgtaaatgcaatgtctgtg |
| | agctgggatacatgggcagtgcttcagac |
| 238834_at | gagagaatcctgcatctaacaattttaattttttnccctntatgctgttattccttacctagagaaacaatttccctccaaag |
| SEQ ID | ttcctttgaggggtctgtttaggccaggccaacacaagtgacctatgtggattttagcatcctttttttgaaatttgaggttt |
| NO: 468 | tatgaagcttgagtttttctggatatttttagtaatttgctggtgtgtacttagctcagatacttgattgcaactgtgttggg |
| | tcaactatttctaatgggacttttccatttgcatgtacagtcactggaaactgctgggcagagaaactctaaaaggtagttgg |
| | ggcacactttttccacctgtcagattggtgaagaattggtgaggctgtggggaaaatggcattctcccactt |
| 241671_x_at | attctgaagccagaccgtctttcctcacagcnnnnnngnnncnnnnccgngggcacgcaagcttcccctggttacctgagctg |
| SEQ ID | ctcctgccgtctcccgcntgggnttcgcngtggtgcacccgatcccggnatcgtgcgtntgcgccctgcgaaagaaggncctg |
| NO: 469 | ctggcggantccggncggggtctcctgnctcgcagctgggcgaggggacttggaggacagggtgaagctgcagaagacctggg |
| | gtgggatggntagagaggacgccaaggactggggaaggggaagttaggaataccttacatccaatgccca |
| 243010_at | gaaatacacccactctcttggaataatgacgtaccactcagttggaccctcaagagtcactgctttgtctgtgctggtagttt |
| SEQ ID | gtgagaagtgacccgcacgcttccatttgatgcatttgatgtgagtgaatccatacatttgaatgtcattgtccttgagaccc |
| NO: 470 | tacatgtgcagtttggctcatctcattaaagatgcttgatgtaataattggttagtttccttttattttcctgcaggcttttc |
| | catgagtattatttttttcaaagaacaaatctgtatggcttttccccatctccatattttgttttgctatgaattgctttgct |
| | ttggtgaacttgtcctagtatgcttgcctcacaaacgttttagccattgtgaattttcttcatctctgtaaatagttcatctg |
| | tgcttctccctgatgacgttttattttttttcccctgtaagcaaccgaggtagaaaaataaattgtttaccatggananannn |
| | nntgctgccgtctcttagcctgacagtgtcctgttctc |
| 244061_at | gaaatggcacattttctggatgtgagagttggtcaaaagatcacaaaaaaagtcaaaaaataattctactctgtgaatgaaaa |
| SEQ ID | atggatatttnngtacttaccctcataagcattaaaagaaaataatgcatgaaattccatagaaatgtgcctatcatgttata |
| NO: 471 | ctgactcaaaccagaagacctagagtatgatattgctaatataatacatgtggtgggtatgagtggaagtatgtgtgtgagat |
| | ttatcattgccatagtgtaaaagagttgaattagcttccacttgactagatgagagctcttagttcttatt |
| 244393_x_at | cccagccgctataacttttaacaattcccatatgtcctttattccactaagatgagtgcagtatatatttccatctgtccaag |
| SEQ ID | gcttcctaaatgtagccaangccaagccaacaccagtcacatgatcnaaatcaaagggcatttggggaatccaggctgtgatt |
| NO: 472 | cagggaagttccaagtgtctgatgaagtgtttgttttacatctttgtgtcccttgcaggtctagcactgtgctatgtaggtaa |
| | catgtgctcc |
| 34210_at SEQ | taatcggctcactataggaatttgcntcgaggccaagattcgnacgagnnngttcaaaagcagctaaaccaaaagaagcctcc |
| ID NO: 473 | agacagccctgagatcacctaaaaagctgctaccaagacagccacgaagatcctaccaaaatgaagcgcttcctcttcctcct |
| | actcaccatcagcctcctggttatggtacagatacaaactggactctcaggacaaaacgacaccagccaaaccagcagcccct |
| | cagcatccagcagcatgagcggaggcattttccttttcttcgtggccaatgccataatccacctcttctgcttcagttgaggt |
| | gacacgtctcagccttagccctgtgccccctgaaacannnnnnnnnnnnnnnnnnnagagaatcccctccatctttgggaggg |
| | gttgatgccagacatcaccaggttgtagaagttgacaggcagtgccatggggncaacagccaaaataggggggtaatgatgta |
| | ggggccaagcagtgcccagctgggggtcaataaagttacccttgtacttg |

TABLE 13
34 PS gene list, target sequences

| | |
|---|---|
| 1552584_at | actgtggccgacctactatttttattatttttgagctnggttctcagtctgttggcagactggagtgcaatcatggctcactg |
| SEQ ID | cagccttgaactcccagactcaagtgatccttccacctcagcctctggagtagctgggactacagacatgcaccaccacacct |
| NO: 474 | ggttaattttttatttttattttttgtngagacaggtgtctcnctacgttgcccaggctggtctcgaactcctgggctcaagt |
| | gatccacccatctccacctcccaaagtgctaggattacaggcgtgagccaccgtacccagcctggtcccatatcatagtgaaatggtgc |
| | ctgtaaagctctcagcattggcttggcacatgcagttggtactcaataaacggctgttgctatcccc |
| 1558972_s_at | ttctaagacccacatttggttattgaaggccacagcgaatcttaacctaacagccttgacaaactgcaccataggtgttttta |
| SEQ ID | gactcatataatttgttatttttcaaacaatagtgaataattaatattnttgtttggaatttgagnacaattaaatttgtact |
| NO: 475 | tttagtaactaccattctttgattagaaaattaagagaatgcatatcttactttggttgtaaattatcaagggctttctaata |
| | gaaatcatatataacatttctaaatataagtcctttcacatactgtgtttccagttgtcttgatattgaaaagtgtaataaacttcatg |
| | ctcacctat |
| 1559584_a_at | attcctgggctcgagagagccactcatccccatctctcagagtgttgggattacagccatgagccaccatgcccggccaaact |
| SEQ ID | tcagccttgaatattagtgattttgccatattagtttaccatctgtacaattatttgctttatntttttctttaaattggctc |
| NO: 476 | attattcacttaatgaaattattgcaacaggaatcttttgtatcacttctgtaaatggaaaaccagtctcatttgccatcaat |
| | agaaggt |
| 1563473_at | gaaaattcctggcagtttcaactgtgatagacattgctaacctgttctccaaagaggctgaaccaatttctgtttcctcaaca |
| SEQ ID | gtgtatgactgtttcccccatctattctccagcactgaggattaagtaactttcatttttgtcagtctgacagatataaagca |
| NO: 477 | gaacatttctgcataaggttctacagtaatttttagattttatgaccctttggattatgcctacataatgatgatcaaatatt |
| | cagaaactacattgtacctggccttaggcttggaattggatacaaaattaaatgaaaccagcttttgccctcaggttgatccc |
| | atctcctggagttggcagacaaatgaacaaataaaatgagagcaaaactgtatggttcacattgtgctagagaaatgcataag |
| | cttagctaacttttgtttgataaactctatattcattaatatcacaaatgaattcataaaataccgtatgcattatgtcccag |
| | gg |
| 203915_at | gattatcaattaccacaccatctcccatgaagaaagggaacggtgaagtactaagcgctagaggaagcagccaagtcggttag |
| SEQ ID | tggaagcatgattggtgcccagttagcctctgcaggatgtggaaacctccttccaggggaggttcagtgaattgtgtaggaga |
| NO: 478 | ggttgtctgtggccagaatttaaacctatactcactttcccaaattgaatcactgctcacactgctgatgatttagagtgctg |
| | tccggtggagatcccacccgaacgtcttatctaatcatgaaactccctagttccttcatgtaacttccctgaaaaatctaagt |
| | gtttcataaatttgagagtctgtgacccacttacc |
| 204116_at | ttctggctggaacggacgatgccccgaattcccaccctgaagaacctagaggatcttgttactgaataccacgggaacttttc |
| SEQ ID | ggcctggagtggtgtgtctaagggactggctgagagtctgcagccagactacagtgaacgactctgcctcgtcagtgagattc |
| NO: 479 | ccccaaaaggaggggcccttggggaggggcctggggcctccccatgcaaccagcatagcccctactgggcccccccatgttac |
| | accctaaagcctgaaacctgaaccccaatcctctgacagaagaaccccagggtcctgtagccctaagtggtactaactttcct |
| | tcattcaacccacctgcgtctcatactcacctcaccccactgtggctgatttggaattttgtgcccccatgtaagcacc |
| 204533_at | taactctaccctggcactataatgtaagctctactgaggtgctatgttcttagtggatgttctgaccctgcttcaaatatttc |
| SEQ ID | cctcacctttcccatcttccaagggtactaaggaatctttctgctttggggtttatcagaattctcagaatctcaaataacta |
| NO: 480 | aaaggtatgcaatcaaatctgctttttaaagaatgctctttacttcatggacttccactgccatcctcccaaggggcccaaat |
| | tctttcagtggctacctacatacaattccaaacacatacaggaaggtagaaatatctgaaaatgtatgtgtaagtattcttat |
| | ttaatgaaagactgtacaaagtataagtcttagatgtatatatttcctatattgttttcagtgtacatggaataacatgtaat |
| | taagtactatgtatcaatgagtaacaggaaaattttaaaaatacagatagatatatgctctgcatgttacataagataaatgt |
| | gctgaatggttttcaaataaaaatgaggtactctcctggaaatatt |
| 205242_at | ggagtttgcattcttattcatcagggaggaaagtttctttgaaaatagttattcagttataagtaatacaggattattttgat |
| SEQ ID | tatatacttgttgtttaatgtttaaaatttcttagaaaacaatggaatgagaatttaagcctcaaatttgaacatgtggcttg |
| NO: 481 | aattaagaagaaaattatggcatatattaaaagcaggcttctatgaaagactcaaaaagctgcctgggaggcagatggaactt |
| | gagcctgtcaagaggcaaaggaatccatgtagtagatatcctctgcttaaaaactcactacggaggagaattaagtcctactt |
| | ttaaagaatttctttataaaatttactgtctaagattaatagcattcgaagatccccagacttcatagaatactcagggaaag |
| | catttaaagggtgatgtacacatgtatcctttcacacatttgccttgacaaacttctttcactcacatctttttcactgactt |
| | tttttgtgggggcggggccggggggactctggtatctaattcttta |
| 205488_at | cagccacacgcgaaggtgaccttaaacttttacagctgacggaaaaagcaaaaattaacaaatatgtgactatccttcatcta |
| SEQ ID | cctaaaaagggggatgatgtgaaaccaggaaccatgtgccaagttgcagggtgggggaggactcacaatagtgcatcttggtc |
| NO: 482 | cgatactctgagagaagtcaatatcaccatcatagacagaaaagtctgcaatgatcgaaatcactataattttaaccctgtga |
| | ttggaatgaatatggtttgtgctggaagcctccgaggtggaagagactcgtgcaatggagattctggaagccctttgttgtgc |
| | gagggtgttttccgaggggtcacttcctttggccttgaaaataaatgcggagaccctcgtgggcctggtgtctatattcttct |
| | ctcaaagaaacacctcaactgga |
| 205758_at | cagcccttgcattgcagaggggcccatgaaagaggacaggctacccctttacaaatagaatttgagcatcagtgaggttaaac |
| SEQ ID | taaggccctcttgaatctctgaatttgagatacaaacatgttcctgggatcactgatgactttttatactttgtaaagacaat |
| NO: 483 | tgttggagagcccctcacacagccctggcctcngctcaactagcagatacagggatgaggcagacctgactctcttaaggagg |
| | ctgagagcccaaactgctgtcccaaacatgcacttccttgcttaaggtatggtacaagcaatgcctgcccattggagagaaaa |
| | aacttaagtagataaggaaataagaaccactcataattcttcaccttaggaataatctcctgttaatatggtgtacattcttc |
| | ctgattattttctacacatac |
| 205890_s_at | gatcttaaagccacggagaagcctctcatcttatggcattgacaaagagaagaccatccaccttaccctgaaagtggtgaagc |
| SEQ ID | ccagtgatgaggagctgcccttgtttcttgtggagtcaggtgatgaggcaaagaggcacctcctccaggtgcgaaggtccagc |
| NO: 484 | tcagtggcacaagtgaaagcaatgatcgagactaagacgggtataatccctgagacccagattgtgacttgcaatggaaagag |
| | actggaagatgggaagatgatggcagattacggcatcagaaagggcaacttactcttcctggcatcttattgtattggagggt |
| | gaccaccctggggatggggtgttggcaggggtcaaaaagcttatttcttttaatctcttactcaacgaacacatcttctgatg |
| | atttcccaaaattaatgagaatgagatgagtagagtaagatttgggtgggatgggtaggatgaagtatattgcccaactctat |
| | gtttctttga |
| 206134_at | ctttctatattgttatcagtccaggaaacaggtaaacagatgtaattagagacattggctctttgtttaggcctaatctttct |
| SEQ ID | ttttacttttttttttcttttttctttttttttaaagatcatgaatttgtgacttagttctgccctttggagaacaaaagaaa |
| NO: 485 | gcagtcttccatcaaatcaccttaaaatgcacggctaaactattcagagttaacactccagaattgttaaattacaagtacta |
| | tgctttaatgcttctttcatcttactagtatggcctataaaaaaaataataccacttgatgggtgaaggctttggcaatagaa |
| | agaagaatagaattcaggttttatgttattcctctgtgttcacttcgccttgctcttgaaagtgcagtatttttctacatcat |
| | gtcgagaatgattcaatgtaaatatttttcattttatcatgtatatcctatacacacatctccttcatcatcatatatgaagt |
| | ttattttgagaagtctacattgcttacattt |
| 206666_at | aaacctctcttagatctggaaccaaatgcaaggttactggctggggagccaccgatccagattcattaagaccttctgacacc |
| SEQ ID | ctgcgagaagtcactgttactgtcctaagtcgaaaactttgcaacagccaaagttactacaacggcgacccttttatcaccaa |
| NO: 486 | agacatggtctgtgcaggagatgccaaaggccagaaggattcctgtaagggtgactcagggggccccttgatctgtaaaggtg |
| | tcttccacgctatagtctctggaggtcatgaatgtggtgttgccacaaagcctggaatctacaccctgttaaccaagaaatac |
| | cagacttggatcaaaagcaaccttgtcccgcctcatacaaattaagttacaaataattttattggatgcacttgcttcttttt |
| | tcctaatatgctcgcaggttagagttgggtgtaagtaaagcagagcacatatggggtccatttttgcacttgta |
| 207651_at | ttgccttgtaattcgacagctctacagaaacaaagataatgaaaattacccaaatgtgaaaaaggctctcatcaacatacttt |
| SEQ ID | tagtgaccacgggctacatcatatgctttgttccttaccacattgtccgaatcccgtataccctcagccagacagaagtcata |
| NO: 487 | actgattgctcaaccaggatttcactcttcaaagccaaagaggctacactgctcctggctgtgtcgaacctgtgctttgatcc |
| | tatcctgtactatcacctctcaaaagcattccgctcaaaggtcactgagacttttgcctcacctaaagagaccaaggctcaga |
| | aagaaaaattaagatgtgaaaataatgcataaaagacaggattttttgtgctaccaattctggccttactgga |
| 209606_at | gaattgcaaaactgacatcccatttcacagcaatagtgacctttatttaaattgttgtgttatagtttatgcttcttaaatca |
| SEQ ID | tttttcaacctaaacagccaatttctaagcagacaggaaaactaaataataagttaattaatataacaaagatgcaggttcct |
| NO: 488 | gctcattccagtaatgtctttgaaagcaaaactaatatttattttctagattatccctgtgaataattgagaactttttggag |
| | tcaagtatgaataaaggtgtggcagaatataataatctggactattttctataggataattgctgggttataaaatcttaggt |
| | ttgcttatgcccagtagctcctgcggaggcttaataataggcaattttgaatttgttcaaacctgtaatggcttgtaaacaaa |
| | gatgaccatcagctgtttctcacatctatagtgacaataaagcgggaagtataagatttaataggaggggttaaggttcatga |
| | gaaccatggaaagatgtggtctgagatgggtgctgcaaagat |
| 209795_at | tagtctaattgaatcccttaaactcagggagcatttataaatggcaaatgcttatgaaactaagatttgtaatatttctctct |
| SEQ ID | ttttagagaaatttgccaatttactttgttatttttccccaaaaagaatgggatgatcgtgtatttatttttttacttcctca |
| NO: 489 | gctgtagacaggtccttttcgatggtacatatttctttgcctttataatcttttatacagtgtcttacagagaaaagacataa |
| | gcaaagactatgaggaatatttgcaagacatagaatagtgttggaaaatgtgcaatatgtgatgtggcaaatctctattagga |
| | aatattctgtaatcttcagacctagaataatactagtcttataataggtttgtgactttcctaaatcaattctattacgtgca |
| | atacttcaatacttcat |
| 210915_x_at | aaaggccacactggtgtgcctggccacaggtatcttccctgaccacgtggagctgagctggtgggtgaatgggaaggaggtgc |
| SEQ ID | acagtggggtcagcacggacccgcagcccctcaaggagcagcccgccctcaatgactccagatactgcctgagcagccgcctg |
| NO: 490 | agggtctcggccaccttctggcagaacccccgcaaccacttccgctgtcaagtccagttctacgggctctcggagaatgacga |
| | gtggacccaggatagggccaaacccgtcacccagatcgtcagcgccgaggcctggggtagagcagactgtggctttacctcgg |
| | tgtcctaccagcaaggggtcctgtctgccaccatcctctatgagatcctgctagggaaggccaccatgtatgctgtgctggtc |
| | agcgcccttgtgttgatggccatggtcaagagaaaggatttctgaaggcagccctggaagtggagttaggagcttctaacccg |
| | tcatggtttcaatacacattcttcttttgccagc |
| 210972_x_at | ggaacaagacttcaggtcacgctcgatatccagaaccctgaccctgccgtgtaccagctgagagactctaaatccagtgacaa |
| SEQ ID | gtctgtctgcctattcaccgattttgattctcaaacaaatgtgtcacaaagtaaggattctgatgtgtatatcacagacaaaa |
| NO: 491 | ctgtgctagacatgaggtctatggacttcaagagcaacagtgctgtggcctggagcaacaaatctgactttgcatgtgcaaac |
| | gccttcaacaacagcattattccagaagacaccttcttccccagcccagaaagttcctgtgatgtcaagctggtcgagaaaag |
| | ctttgaaacagatacgaacctaaactttcaaaacctgtcagtgattgggttccgaatcctcctcctgaaagtggccgggttta |
| | atctgctcatgacgctgcggctgtggtccagctgagatctgcaagattgtaagacagcctgtgctccct |
| 211796_s_at | gccatcagaagcagagatctcccacacccaaaaggccacactggtgtgcctggccacaggtttctaccccgaccacgtggagc |
| SEQ ID | tgagctggtgggtgaatgggaaggaggtgcacagtggggtcagcacagacccgcagcccctcaaggagcagcccgccctcaat |
| NO: 492 | gactccagatactgcctgagcagccgcctgagggtctcggccaccttctggcagaacccccgcaaccacttccgctgtcaagt |
| | ccagttctacgggctctcggagaatgacgagtggacccaggatagggccaaacctgtcacccagatcgtcagcgccgaggcct |
| | ggggtagagcagactgtggcttcacctccgagtcttaccagcaaggggtcctgtctgccaccatcctctatgagatcttgcta |
| | gggaaggccaccttgtatgctgtgctggtcagtgccctcgtgctgatggccatggtcaagagaaagga |
| 212592_at | gtatcaaaatcttccaattatcatgctcacctgaaagaggtatgctctcttaggaatacagtttctagcattaaacaaataaa |
| SEQ ID | caaggggagaaaataaaactcaaggagtgaaaatcaggaggtgtaataaaatgttcctcgcattcccccccgcnttttttttt |
| NO: 493 | ttttttgactttgccttggagagccagagcttccgcattttctttactattctttttaaaaaaagtttcactgtgtagagaac |
| | atatatgcataaacataggtcaattatatgtctccattagaaaaataataattggaaaacatgttctagaactagttacaaaa |
| | ataatttaaggtgaaatctctaatatttataaaagtagcaaaataaatgcataattaaaatatatttggacataacagactt |
| 212999_x_at | cactgcagaatgaaggaacatcccttgaggtgacccagccaacctgtggccagaaggaggnttgtaccttgaaaagacactga |
| SEQ ID | aagcattttggngtgtnaagtaagggtgggcagaggaggtagaaaatcaattcaattgtcgcatcattcatggttctttaata |
| NO: 494 | ttgatgctcagtgcantggcctnagaatatcccagcctctcttctggtttgntgagtgctntntaagtaagcatggtngaatt |
| | gtttggggncanatatagtganccttggtcactggtgtttcaaacattctggnaagtcacatcnatcaagaatantttttant |
| | tttaagaaagcataaccagcaataaa |
| 213193_x_at | tgactccagatactgcctgagcagccgcctgagggtctcggccaccttctggcagaacccccgcaaccacttccgctgtcaag |
| SEQ ID | tccagttctacgggctctcggagaatgacgagtggacccaggatagggccaaacccgtcacccagatcgtcagcgccgaggcc |
| NO: 495 | tggggtagagcagactgtggctttacctcggtgtcctaccagcaaggggtcctgtctgccaccatcctctatgagatcctgct |
| | agggaaggccaccctgtatgctgtgctggtcagcncccttgtgttgatggccatggtcaagagaaaggatttctgaaggcagc |
| | cctggaagtggagttaggagcttctaacccgtcatggtttcaatacacattcttcttttgccagcgcttctgaagagctgctc |
| | tcacctctctgcatcccaatagatatccccctatgtgcatgcacacctgcacactcacggctgaaatctccctaacccagggg |
| | gaccttagcatgcctaagtga |
| 213539_at | gggaacactgctctcagacattacaagactggacctgggaaaacgcatcctggacccacgaggaatatataggtgtaatggga |
| SEQ ID | cagatatatacaaggacaaagaatctaccgtgcaagttcattatcgaatgtgccagagctgtgtggagctggatccagccacc |
| NO: 496 | gtggctggcatcattgtcactgatgtcattgccactctgctccttgctttgggagtcttctgctttgctggacatgagactgg |
| | aaggctgtctggggctgccgacacacaagctctgttgaggaatgaccaggtctatcagcccctccgagatcgagatgatgctc |
| | agtacagccaccttggaggaaactgggctcggaacaagtgaacctgagactggtggcttctagaagcagccattaccaactgt |
| | acct |
| 214617_at | ccaacgcaaattcgcaaactttcttaaaacattatgagttncnntttgctatttttttttttttttttagctcatcggctatc |
| SEQ ID | gttagtgctagtggattttacatgtggcccnnnannnnnnnncnnncaacgtggcccagagaagccaaaagattggatacgca |
| NO: 497 | tcagacagatggaaaagggagattcagactgtttttcagggaggtggctgggtttacacgctaatcccgattcaccctgtcca |
| | aactgcctaagccctccgccattntcaagccctgcagtcacagctacacagatcacagcttcagccaggagctgggcagaagg |
| | ccaanaggctgttcccaccaggctgctcagggntggtcttttaggacccttcccttgagccctntatggtgtggcaaagcctt |
| | cattgccttaactggagccccatcagctccagctgctctgtnttntttgcccncaatgctttgcccctgagacaaatggaggc |
| | ctgtcctgacctgtctcaccatgtacatagctt |
| 216920_s_at | cactactgctgcagctcacaaacacctctgcatattacatgtacctcctcctgctcctcaagagtgtggtctattttgccatc |
| SEQ ID | atcacctgctgtctgcttngaagaacggctttctgctgcaatggagagaaatcataacagacggtggcacaaggaggccatct |
| NO: 498 | tttcctcatcggttattgtccctagaagcgtcnncnnannnnnnnnttgggctttctttctgggtttgggccatttcagttct |
| | catgtgtgtactattctatctattgtataatggttttcaaaccagtgggcacacagagaacctcactctgtaataacaatgag |
| | gaatagccatggcgatctccagcaccaatctctccatgttttccacagctcctccagccaacccaaatagcgcctgctatagt |
| | gtagacagcctgcggcttctagccttgtccctctcttagtgttctttaatcagataactgcctggaagcctttcattttacac |
| | gccctgaagcagtcttctttgctagttgaattatgtggtgtgtttttccgtaata |
| 217147_s_at | tctcctttctcaccaatgggcaatagcccataattgaaataaatttctgattgaaaggtataggaaacattaaaatgcattac |
| SEQ ID | taagagaagtaatataattttcttacaaagtatttttcccaaagatagctttactatttcaaaaattgtcaaattaatgcatg |
| NO: 499 | ctccttacaacaaacaaatatcaaaaagagtttaggaattctactagccagagatagtcacttggagaaactttctatatatc |
| | cttctaaatatttttctgggcatgctcatgtatgtacatcagttgtttctttttattttgaaccaaaaatgtggtttcttttg |
| | tacacattacttaaactttctttccagtcaacaatatattgtggatttattttcactgttatatttaactatatataaatacg |
| | catatattgtaattttaatgtctgcttagcaccccactgataaccaaatcacag |
| 222838_at | aacacctgtgctaggtcagtctggcacgtaagatgaacatccctaccaacacagagctcaccatctcttatacttaagtgaaa |
| SEQ ID | aacatggggaaggggaaaggggaatggctgcttttgatatgttccctgacacatatcttgaatggagacctccctaccaagtg |
| NO: 500 | atgaaagtgttgaaaaacttaataacaaatgcttgttgggcaagaatgggattgaggattatcttctctcagaaaggcattgt |
| | gaaggaattgagccagatctctctccctactgcaaaaccctattgtagta |
| 222895_s_at | gttctctcatttaatgcacactgatgatattgtagggatgggtggggtggggatcttgcaaatttctattctcttttactgaa |
| SEQ ID | aaagcaggggatgagttccatcagaaggtgcccagcgctacttcccaggtttttattttttttttcctatctcattaggttgg |
| NO: 501 | aaggtactaaatattgaactgttaagattagacatttgaattctgttgacccgcactttaaagcttttgtttgcatttaaatt |
| | aaatggcttctaaacaagaaattgcagcatattcttctctttggcccagaggtgggttaaactgtaagggacagctgagattg |
| | agtgtcagtattgctaagcgtggcattcacaatactggcactataaagaacaaaataaaataataatttataggacagttttt |
| | ctactgccattcaatttgatgtgagtgccttgaaaactgatcttcctatttgagtctcttgaga |
| 226218_at | ctaattggttctgcccaatctcctttcagattttattaggaaaaaaaaataaacctcctgatcggagacaatgtattaatcag |
| SEQ ID | aagtgtaaactgccagttctatatagcatgaaatgaaaagacagctaatttggtccaacaaacatgactgggtctagggcacc |
| NO: 502 | caggctgattcagctgatttcctaccagcctttgcctcttccttcaatgtggtttccatgggaatttgcttcagaaaagccaa |
| | gtatgggctgttcagaggtgcacacctgcattttctnagctcttctagaggggctaagagacttggtacgggccaggaagaat |
| | atgtggcagagctcctggaaatgatgcagattaggtggcatttttgtcagctctgtggtttattgttgggactattctttaaa |
| | atatccattgttcactacagtgaagatctctgatttnaccgtgtactatccacatgcattacaaacatttcgcagagctgct |
| 227346_at | tgcttttggtgcgatggcactcactgtgaacatgtgtaaccacatattaatatgcaatattgtttccaatactttctaataca |
| SEQ ID | gttttttataatgttgngtgtggtgattgttcaggtcgaatctgttgtatccagtacagctttaggtcttcagctgcccttct |
| NO: 503 | ggcgagtacatgcacaggattgtaaatgagaaatgcagtcatatttccagtctgcctctatgatgatgttaaattattgctgt |
| | ttagctgtgaacaagggatgtaccactggaggaatagagtatccttttgtacacattttgaaatgcttcttctgtagtgatag |
| | aac |
| 228362_s_at | gtactggcccttcggattgaaagtatacagtgatgaaatttgctgccactctttcatgcttggagtgttatattcttttggat |
| SEQ ID | gcgagccctcaaagaaacatttaatattctcttttgccaattcagttgcatgctctgtggctttacttttaaggatctgctgc |
| NO: 504 | tcctgttccaaatagattttccagaatttcagctgcagaaaactaactggagataggcatcgggtgacagatgtaaaaatcag |
| | aagaatgatgataacaactgctatcaagatccagcccaac |
| 229152_at | tgaagaaagttctcctcctgatcacagccatcttggcagtggctgttggtttcccagtctctcaagaccaggaacgagaaaaa |
| SEQ ID | agaagtatcagtgacagcgatgaattagcttcagggttttttgtgttcccttacccatatccatttcgcccacttccaccaat |
| NO: 505 | tccatttccaagatttccatggtttagacgtaattttcctattccaatacctgaatctgcccctacaactccccttcctagcg |
| | aaaagtaaacaagaaggaaaagtcacgataaacctggtcacctgaaattgaaattgagccacttccttgaagaatcaaaattc |
| | ctgttaataaaagaaaaacaaatgtaattgaaatagcacacagcattctctagtcaatatctttagtgatcttctttaata |
| 231577_s_at | ggaacaggagcaactactaaaagagggatttcaaaaagaaagcagaataatgaaaaatgagatacaggatctccagacgaaaa |
| SEQ ID | tgagacgacgaaaggcatgtaccataagctaaagaccagagccttcctgtcacccctaaccaaggcataattgaaacaatttt |
| NO: 506 | agaatttggaacaagcgtcactacatttgataataattagatcttgcatcataacaccaaaagtttataaaggcatgtggtac |
| | aatgatcaaaatc |
| 236280_at | aaaagatctctcactgggaaaagaaaaagttatgcatttataaagtaattaaactggttttccttgtactttattaatctgaa |
| SEQ ID | tctaatggcacttccttacgagggttttcagatgtgcttgtagttaatggcaacattatcagaatgactacacagacagtcct |
| NO: 507 | actctgaggagatgactttggaagaaacccatttggaactacacaccctgctatgtctgtggagaaatggaactgcaatcctc |
| | aagagtcacacttcatattccttcctttcaagtggttgataaaaggtagtgcttcaagcacaggatttatggaatagttggca |
| | aattaaacaacatgctttttattttgactacca |

TABLE 14
18 PS gene list probe sequences

| | | | | | | |
|---|---|---|---|---|---|---|
| | | | Probe | | | |
| Probe Set | | | Interrogation | | Target | |
| Name | Probe.X | Probe.Y | Position | Probe Sequence | Strandedness | SEQ ID NO: |

| | | | | | | |
|---|---|---|---|---|---|---|
| 1562031_at | 1147 | 61 | 1892 | ATGTTCACTGTATGTGCCAAGCCTA | Antisense | 508 |
| 1562031_at | 289 | 165 | 1898 | ACTGTATGTGCCAAGCCTAATATGA | Antisense | 509 |
| 1562031_at | 811 | 761 | 1905 | GTGCCAAGCCTAATATGAGAGCTAT | Antisense | 510 |
| 1562031_at | 374 | 1103 | 1933 | TTATAGAGTTTATGCTACAGCCCTA | Antisense | 511 |
| 1562031_at | 1143 | 113 | 1937 | AGAGTTTATGCTACAGCCCTACCTT | Antisense | 512 |
| 1562031_at | 529 | 189 | 1949 | ACAGCCCTACCTTCAGGAAACTTAT | Antisense | 513 |
| 1562031_at | 743 | 77 | 1963 | AGGAAACTTATCTACTGGACAAACA | Antisense | 514 |
| 1562031_at | 377 | 179 | 2022 | ACATTGTAATTATCTAGCATAGGCA | Antisense | 515 |
| 1562031_at | 519 | 519 | 2038 | GCATAGGCAAATATAGACAGTAACA | Antisense | 516 |
| 1562031_at | 968 | 229 | 2059 | AACAGACAGGTTTACAATTATTAAG | Antisense | 517 |
| 1562031_at | 442 | 293 | 2073 | CAATTATTAAGAAAGGGCAGCCAGG | Antisense | 518 |
| 1563497_at | 251 | 1063 | 4804 | TACCATAGCAAGTTCTTATCCTTTG | Antisense | 519 |
| 1563497_at | 173 | 157 | 4861 | ACCTGTCACAGTAATATGCCAATTT | Antisense | 520 |
| 1563497_at | 943 | 467 | 4878 | GCCAATTTTTGGTGTCGCTTTAGTA | Antisense | 521 |
| 1563497_at | 834 | 749 | 4965 | GTCTTTCCTCAGTTGTCTCCTAAAA | Antisense | 522 |
| 1563497_at | 555 | 677 | 5022 | GATCGTCTTGTGCAGCCCTTTAAAA | Antisense | 523 |
| 1563497_at | 941 | 53 | 5048 | ATGCGTTTTGCATTCATGTCTTTGC | Antisense | 524 |
| 1563497_at | 920 | 365 | 5088 | CTTTCATTTTGGGAGTGACTGTCAT | Antisense | 525 |
| 1563497_at | 1079 | 189 | 5141 | ACACCAGGGCCAGATTGCGTGGGTT | Antisense | 526 |
| 1563497_at | 610 | 965 | 5156 | TGCGTGGGTTCTAGCTAGGGACCTA | Antisense | 527 |
| 1563497_at | 1053 | 141 | 5182 | AGCTGGTTACTTGATTTCTCTCTGT | Antisense | 528 |
| 1563497_at | 93 | 1137 | 5197 | TTCTCTCTGTTCCATTTACTATCTG | Antisense | 529 |
| 202290_at | 1042 | 87 | 267 | AGGGCTCATCGACATCGAGAACCCC | Antisense | 530 |
| 202290_at | 470 | 825 | 318 | GGTCACACAACTGGATCTGGACGGG | Antisense | 531 |
| 202290_at | 672 | 839 | 336 | GGACGGGCCAAAGGAGCTTTCGAGG | Antisense | 532 |
| 202290_at | 567 | 51 | 412 | ATGCACTTGGCCGGGAAGACAGAGC | Antisense | 533 |
| 202290_at | 1035 | 885 | 458 | GGCTGGCCATCATCCGGAAACAGCG | Antisense | 534 |
| 202290_at | 550 | 659 | 484 | GAGGAGGCTGCCCGGAAGAAGGAAG | Antisense | 535 |
| 202290_at | 101 | 211 | 523 | AAAGACGATGCCACATTGTCAGGAA | Antisense | 536 |
| 202290_at | 907 | 861 | 544 | GGAAAACGAATGCAGTCACTCTCCC | Antisense | 537 |
| 202290_at | 601 | 529 | 555 | GCAGTCACTCTCCCTGAATAAGTAA | Antisense | 538 |
| 202290_at | 637 | 1039 | 573 | TAAGTAACTGCGACCCGTGGGAGGA | Antisense | 539 |
| 202290_at | 105 | 813 | 764 | GGGGTAGCTGCTATCTTTGAGACAG | Antisense | 540 |
| 203992_s_at | 114 | 77 | 4286 | AGGAAATAACCCAGTTCTGCACCAC | Antisense | 541 |
| 203992_s_at | 245 | 463 | 4307 | CCACTGGTTTTTGTAGCTATCTCGT | Antisense | 542 |
| 203992_s_at | 1100 | 367 | 4323 | CTATCTCGTAAGGCTGCTGGCTGAA | Antisense | 543 |
| 203992_s_at | 261 | 397 | 4343 | CTGAAAACTGTGTCTATGCAACCTT | Antisense | 544 |
| 203992_s_at | 578 | 543 | 4360 | GCAACCTTCCAAGTGCGGAGTGTCA | Antisense | 545 |
| 203992_s_at | 92 | 495 | 4374 | GCGGAGTGTCAACCAACTGGACGGG | Antisense | 546 |
| 203992_s_at | 443 | 411 | 4395 | CGGGAGAGAGTACTGCTCCTACTCC | Antisense | 547 |
| 203992_s_at | 638 | 373 | 4413 | CTACTCCAGGACTCTCACAAAGCTG | Antisense | 548 |
| 203992_s_at | 124 | 239 | 4494 | AACTGGCAACATCTTACAGACTACT | Antisense | 549 |
| 203992_s_at | 416 | 611 | 4512 | GACTACTGACTTGAAGACAACCTCT | Antisense | 550 |
| 203992_s_at | 711 | 59 | 4754 | ATGATGGTTACACTTTTGGTTCCTA | Antisense | 551 |
| 205518_s_at | 831 | 1027 | 1672 | TCAAGAATTCACTGATCTGATGCAA | Antisense | 552 |
| 205518_s_at | 760 | 1079 | 1707 | TATCATTACATCTTGAACCCAGGAA | Antisense | 553 |
| 205518_s_at | 705 | 1 | 1769 | ATAGATAATTCCACGTTGCCTTTGT | Antisense | 554 |
| 205518_s_at | 470 | 163 | 1781 | ACGTTGCCTTTGTGATTTGTATATA | Antisense | 555 |
| 205518_s_at | 429 | 23 | 1846 | ATATTTTACTTGTTCCATCCACAGT | Antisense | 556 |
| 205518_s_at | 1070 | 1145 | 1858 | TTCCATCCACAGTTCTCTACAGAAA | Antisense | 557 |
| 205518_s_at | 583 | 271 | 2006 | AATGGTCTGTTTTCGTGATATTCGT | Antisense | 558 |
| 205518_s_at | 962 | 707 | 2070 | GTTAACTAGCATTCTGTTTTACATG | Antisense | 559 |
| 205518_s_at | 263 | 345 | 2091 | CATGTTGACATTTCTAACACACACA | Antisense | 560 |
| 205518_s_at | 1128 | 307 | 2108 | CACACACACCACTGATTTGAACTTC | Antisense | 561 |
| 205518_s_at | 818 | 21 | 2151 | ATATATGCTAGGTCTGATTCTGAAG | Antisense | 562 |
| 206618_at | 1048 | 569 | 2935 | GAAGCATATCCAGAGGGCGAAAGAT | Antisense | 563 |
| 206618_at | 622 | 885 | 2950 | GGCGAAAGATATCTCTCCATTGTGC | Antisense | 564 |
| 206618_at | 25 | 349 | 2974 | CATCTGCCTCTTTTGACGTTGGAAG | Antisense | 565 |
| 206618_at | 812 | 865 | 2994 | GGAAGACACATGTCTTACTCCCCAA | Antisense | 566 |
| 206618_at | 498 | 1057 | 3009 | TACTCCCCAAAGGGAGCCCAGCACT | Antisense | 567 |
| 206618_at | 317 | 853 | 3035 | GGAGCCTTCTTGATGATCTCAAAAA | Antisense | 568 |
| 206618_at | 3 | 771 | 3086 | GTGACTGTGAAACCGTCAGTTCGGA | Antisense | 569 |
| 206618_at | 182 | 589 | 3094 | GAAACCGTCAGTTCGGAAGGCTGGT | Antisense | 570 |
| 206618_at | 601 | 559 | 3109 | GAAGGCTGGTTAGAACATGTGGGAG | Antisense | 571 |
| 206618_at | 804 | 765 | 3253 | GTGCTTACAATTTTGTGTACCAACT | Antisense | 572 |
| 206618_at | 118 | 689 | 3364 | GATTTCATTGCTATTGCATAGTCTC | Antisense | 573 |
| 212684_at | 268 | 611 | 1158 | GACTATTTCAAAGCTACTGTTCCTA | Antisense | 574 |
| 212684_at | 555 | 699 | 1176 | GTTCCTAGTCCAGCTTTAAGTTTCG | Antisense | 575 |
| 212684_at | 683 | 499 | 1212 | GCTGTTTTGTTTCATGATTTCGTTA | Antisense | 576 |
| 212684_at | 414 | 671 | 1305 | GATGATGGTGTCCTTTAGGGCTCTT | Antisense | 577 |
| 212684_at | 1067 | 89 | 1321 | AGGGCTCTTGGAGCAGCCAGACCAT | Antisense | 578 |
| 212684_at | 957 | 103 | 1399 | AGTTGTCCTGGGGCTGAATGGGCAA | Antisense | 579 |
| 212684_at | 1098 | 557 | 1414 | GAATGGGCAAATCTGTCCAAACAGC | Antisense | 580 |
| 212684_at | 596 | 495 | 1437 | GCTAGTAACCGGCTGTGAGGGAGAG | Antisense | 581 |
| 212684_at | 844 | 745 | 1463 | GTCAGAAGCACTTAGCGTTGGCCTC | Antisense | 582 |
| 212684_at | 377 | 61 | 1532 | ATGATTTTCTCTAAATGCCTGGGTA | Antisense | 583 |
| 212684_at | 148 | 1127 | 1565 | TTCAAGGAGCTCACTTGGCCTGCTT | Antisense | 584 |
| 212776_s_at | 63 | 1003 | 2263 | TCTCCTCGTGGATCGTGTATCCCAG | Antisense | 585 |
| 212776_s_at | 507 | 983 | 2265 | TCCTCGTGGATCGTGTATCCCAGCG | Antisense | 586 |
| 212776_s_at | 1108 | 411 | 2276 | CGTGTATCCCAGCGGCAAGGTGTAT | Antisense | 587 |
| 212776_s_at | 210 | 723 | 2279 | GTATCCCAGCGGCAAGGTGTATGTG | Antisense | 588 |
| 212776_s_at | 163 | 461 | 2284 | CCAGCGGCAAGGTGTATGTGGCAGC | Antisense | 589 |
| 212776_s_at | 1127 | 915 | 2324 | TGTGGTGCTGACCTGTGAGCTATGC | Antisense | 590 |
| 212776_s_at | 404 | 531 | 2358 | GCAGAGGTGCGCTGGACCAAGGATG | Antisense | 591 |
| 212776_s_at | 4 | 129 | 2461 | AGCTCGAGGACTCCGGCGAGTACTT | Antisense | 592 |
| 212776_s_at | 921 | 411 | 2465 | CGAGGACTCCGGCGAGTACTTGTGT | Antisense | 593 |
| 212776_s_at | 512 | 613 | 2469 | GACTCCGGCGAGTACTTGTGTGAAA | Antisense | 594 |
| 212776_s_at | 362 | 197 | 2491 | AAATTGACGATGAGTCGGCCTCCTT | Antisense | 595 |
| 215666_at | 26 | 587 | 760 | GAAAAATCCGTGGAACTGAAGCTGA | Antisense | 596 |
| 215666_at | 830 | 167 | 774 | ACTGAAGCTGAGATCTTTAGTACGT | Antisense | 597 |
| 215666_at | 643 | 643 | 783 | GAGATCTTTAGTACGTGGAGTCACC | Antisense | 598 |
| 215666_at | 618 | 733 | 793 | GTACGTGGAGTCACCTTACAGATAC | Antisense | 599 |
| 215666_at | 1159 | 845 | 799 | GGAGTCACCTTACAGATACAGAGCA | Antisense | 600 |
| 215666_at | 560 | 1025 | 803 | TCACCTTACAGATACAGAGCATTTA | Antisense | 601 |
| 215666_at | 373 | 631 | 819 | GAGCATTTATGCGGTATTCATTGGT | Antisense | 602 |
| 215666_at | 288 | 1079 | 826 | TATGCGGTATTCATTGGTGCCTAAA | Antisense | 603 |
| 215666_at | 878 | 717 | 832 | GTATTCATTGGTGCCTAAAGAACTT | Antisense | 604 |
| 215666_at | 1006 | 209 | 848 | AAAGAACTTTAGGCATCCTCTGGAA | Antisense | 605 |
| 215666_at | 66 | 1117 | 856 | TTAGGCATCCTCTGGAAAACCGGCC | Antisense | 606 |
| 219551_at | 696 | 579 | 285 | GAACAGGTGACCATAACTCTGCCAA | Antisense | 607 |
| 219551_at | 242 | 67 | 370 | ATGTGGAATTCAGCCAGGACTCCCA | Antisense | 608 |
| 219551_at | 56 | 473 | 382 | GCCAGGACTCCCAATCTTGTAAAAC | Antisense | 609 |
| 219551_at | 709 | 107 | 420 | AGATAAGATGTCCCCAGCATCTCCA | Antisense | 610 |
| 219551_at | 925 | 103 | 511 | AGTTCATCAGATTCCAAAAGTTCAT | Antisense | 611 |
| 219551_at | 263 | 259 | 528 | AAGTTCATCATCTTCAAGTAGTGAG | Antisense | 612 |
| 219551_at | 877 | 693 | 580 | GATTGCAAATCCTCTACTTCTGATA | Antisense | 613 |
| 219551_at | 470 | 547 | 609 | GAATTGTGTCTCAGGACATCCTACC | Antisense | 614 |
| 219551_at | 976 | 605 | 623 | GACATCCTACCATGACACAGTACAG | Antisense | 615 |
| 219551_at | 529 | 107 | 676 | AGATTTCGAGACAACAGTGGCCTTC | Antisense | 616 |
| 219551_at | 542 | 185 | 689 | ACAGTGGCCTTCTGATGAATACTTT | Antisense | 617 |
| 220301_at | 373 | 35 | 1467 | ATAAGTCATTTCTAATCTTTGTATA | Antisense | 618 |
| 220301_at | 603 | 605 | 1572 | GACAGGCTGTAAGCATCGCTGAGAA | Antisense | 619 |
| 220301_at | 1151 | 77 | 1603 | AGGACTTTTGACTTTTATCTGGATA | Antisense | 620 |
| 220301_at | 333 | 39 | 1645 | ATCATGGAAAGGCATCAGCATTGCA | Antisense | 621 |
| 220301_at | 931 | 519 | 1675 | GCATCTAGGTAGAAATCAGGCCAAA | Antisense | 622 |
| 220301_at | 70 | 303 | 1696 | CAAAATTAAGCTGTGGTTTCCCTCT | Antisense | 623 |
| 220301_at | 417 | 829 | 1710 | GGTTTCCCTCTGAGTAGTGGGAATA | Antisense | 624 |
| 220301_at | 313 | 253 | 1851 | AAGACACTTATAATTTTCCATACCT | Antisense | 625 |
| 220301_at | 975 | 15 | 1863 | ATTTTCCATACCTATTTTCAACTGA | Antisense | 626 |
| 220301_at | 1035 | 543 | 1890 | GCAACTTGTAAGATTTAACTCAGTC | Antisense | 627 |
| 220301_at | 759 | 293 | 1914 | CAATAACATACTGGTTTTACTCATC | Antisense | 628 |
| 223095_at | 224 | 971 | 2789 | TGCCTGTAGCATTCCAGAGCTCACT | Antisense | 629 |
| 223095_at | 330 | 377 | 2858 | CTCACTCTGTCTGCCAGGTATGAGA | Antisense | 630 |
| 223095_at | 223 | 127 | 2880 | AGAAGAACACGTAAGACCGCCACCA | Antisense | 631 |
| 223095_at | 975 | 391 | 2952 | CTGCCCCCAGAACTTTTGGATACTG | Antisense | 632 |
| 223095_at | 448 | 843 | 2976 | GGAGGCAGTTGCATAGGTCTCCCTC | Antisense | 633 |
| 223095_at | 239 | 489 | 3024 | GCCCAAGACTACTCTGGGCAGCTCC | Antisense | 634 |
| 223095_at | 148 | 861 | 3079 | GGAAAGGATTTCTACAGTGTTCTAT | Antisense | 635 |
| 223095_at | 417 | 115 | 3114 | AGAGAGAGTGGGTTTGGGAAGAGTG | Antisense | 636 |
| 223095_at | 141 | 697 | 3145 | GTTGGGGAGAGGGGACCGATGTGCC | Antisense | 637 |
| 223095_at | 136 | 621 | 3158 | GACCGATGTGCCTCATTGTTTAGTG | Antisense | 638 |
| 223095_at | 581 | 1139 | 3201 | TTCTGGATAAAGTTTGGTTGTTTGC | Antisense | 639 |
| 227927_at | 779 | 1061 | 1233 | TACCTCTGTCGACCTGTGAAACTGA | Antisense | 640 |
| 227927_at | 587 | 445 | 1235 | CCTCTGTCGACCTGTGAAACTGAAC | Antisense | 641 |
| 227927_at | 525 | 589 | 1250 | GAAACTGAACAAACAGCTTATCTGC | Antisense | 642 |
| 227927_at | 631 | 515 | 1265 | GCTTATCTGCCCCTAATGTGAAATG | Antisense | 643 |
| 227927_at | 230 | 365 | 1266 | CTTATCTGCCCCTAATGTGAAATGA | Antisense | 644 |
| 227927_at | 753 | 45 | 1269 | ATCTGCCCCTAATGTGAAATGATGG | Antisense | 645 |
| 227927_at | 467 | 485 | 1273 | GCCCCTAATGTGAAATGATGGGACA | Antisense | 646 |
| 227927_at | 867 | 277 | 1400 | AATCCAGCAGGAGTTTCTTAGTTAG | Antisense | 647 |
| 227927_at | 1130 | 715 | 1412 | GTTTCTTAGTTAGGATCCACAGCCT | Antisense | 648 |
| 227927_at | 811 | 363 | 1416 | CTTAGTTAGGATCCACAGCCTGGGA | Antisense | 649 |
| 227927_at | 909 | 999 | 1483 | TCTGCATTTCTTGAAACCATTATTA | Antisense | 650 |
| 228128_x_at | 382 | 667 | 281 | GATGTTGAAACCTGTTTGGCACCTT | Antisense | 651 |
| 228128_x_at | 666 | 719 | 294 | GTTTGGCACCTTCTGGAAGCTACCA | Antisense | 652 |
| 228128_x_at | 1075 | 349 | 358 | CATAAGAATTCTACTGGCCTATTGT | Antisense | 653 |
| 228128_x_at | 690 | 219 | 447 | AAACACAATTCCAATCCTTTTTCTG | Antisense | 654 |
| 228128_x_at | 199 | 839 | 529 | GGATCTAAGTTAAATCTTTTGGCAA | Antisense | 655 |
| 228128_x_at | 341 | 173 | 661 | ACTCAGACATATTTTGCTGTTCATG | Antisense | 656 |
| 228128_x_at | 6 | 441 | 696 | CCTGTTCTCATGGATTTGTTTTCCC | Antisense | 657 |
| 228128_x_at | 714 | 687 | 708 | GATTTGTTTTCCCATACTGTTTTCT | Antisense | 658 |
| 228128_x_at | 157 | 351 | 720 | CATACTGTTTTCTCTGATCTCAATT | Antisense | 659 |
| 228128_x_at | 996 | 43 | 736 | ATCTCAATTACAGGTTGGATCTCAC | Antisense | 660 |
| 228128_x_at | 618 | 15 | 786 | ATTTTGCCACTGTTGATTACTATAC | Antisense | 661 |
| 228812_at | 843 | 637 | 765 | GAGACTTCATTGGTGATACACTCAA | Antisense | 662 |
| 228812_at | 406 | 775 | 777 | GTGATACACTCAATTTTTACTGGGT | Antisense | 663 |
| 228812_at | 488 | 509 | 807 | GCTAATAATGTTGGTCACTGTCTCA | Antisense | 664 |
| 228812_at | 742 | 301 | 869 | CACAAAGCTTTTGGGTAACCAGCGT | Antisense | 665 |
| 228812_at | 873 | 805 | 881 | GGGTAACCAGCGTTCTTAAATGTAT | Antisense | 666 |
| 228812_at | 552 | 1075 | 903 | TATGGTTTTTGACCAGGTGAACCCT | Antisense | 667 |
| 228812_at | 775 | 623 | 913 | GACCAGGTGAACCCTTTAGAAGTGA | Antisense | 668 |
| 228812_at | 1155 | 199 | 963 | AAATACCTTTGGCTGTGATGAATGT | Antisense | 669 |
| 228812_at | 1101 | 953 | 981 | TGAATGTAGATCCCAGCAGAATACC | Antisense | 670 |
| 228812_at | 137 | 1153 | 1021 | TTGACTGAGTATTTGTAGATGCTTA | Antisense | 671 |
| 228812_at | 198 | 259 | 1091 | AAGTTCTCAGGTACTGTTCAATTAT | Antisense | 672 |
| 229022_at | 446 | 587 | 826 | GAAACACTTTTAGAGCACTTGAGGA | Antisense | 673 |
| 229022_at | 167 | 27 | 937 | ATAGGACCCCAGACAGTTTATACCT | Antisense | 674 |
| 229022_at | 124 | 715 | 952 | GTTTATACCTTCCATTTGCTGTTTT | Antisense | 675 |
| 229022_at | 697 | 263 | 979 | AAGTGTGAGCCTGCCAAGTCAACAA | Antisense | 676 |
| 229022_at | 990 | 479 | 987 | GCCTGCCAAGTCAACAAGTATGCCT | Antisense | 677 |
| 229022_at | 645 | 297 | 1001 | CAAGTATGCCTTTAGCGCACATGTA | Antisense | 678 |
| 229022_at | 328 | 311 | 1018 | CACATGTAAATAGCCTGCACTTCCT | Antisense | 679 |
| 229022_at | 744 | 539 | 1034 | GCACTTCCTAAATCTCGTGTGGCCT | Antisense | 680 |
| 229022_at | 835 | 15 | 1195 | ATTTTGCCACATTCTGTAACTGTTT | Antisense | 681 |
| 229022_at | 529 | 1101 | 1256 | TTTCATCTTTTGTTTCGTGTATATA | Antisense | 682 |
| 229022_at | 845 | 777 | 1272 | GTGTATATACTGTTTGCCTTTTTCA | Antisense | 683 |
| 233976_at | 677 | 939 | 1736 | TGAGTCTTTTGAACACAGGTATGAA | Antisense | 684 |
| 233976_at | 225 | 1007 | 1773 | TCTAAACCAATGTTACCCCTACCAT | Antisense | 685 |
| 233976_at | 757 | 707 | 1784 | GTTACCCCTACCATACAGGTATGTT | Antisense | 686 |
| 233976_at | 975 | 243 | 1874 | AACCACATTAAATATTCTGTCCATT | Antisense | 687 |
| 233976_at | 985 | 957 | 1951 | TGCAGCATGCAAGGCAAGATGACAA | Antisense | 688 |
| 233976_at | 76 | 201 | 2024 | AAATAATTATGACATGCTTTCTGAA | Antisense | 689 |
| 233976_at | 948 | 261 | 2047 | AAGTGTCCCCAGATAGTTTCATAAA | Antisense | 690 |
| 233976_at | 127 | 587 | 2093 | GAAAAACCCATAGACATCATGTTCA | Antisense | 691 |
| 233976_at | 1001 | 935 | 2125 | TGATGACAAGGCATGTTCATAACCC | Antisense | 692 |
| 233976_at | 725 | 313 | 2168 | CAGCTAAATAACTGCAATACTTTTG | Antisense | 693 |
| 233976_at | 781 | 647 | 2196 | GAGTTTGGCAGGTTTCGAAGATGTG | Antisense | 694 |
| 244008_at | 1036 | 1053 | 112 | TACATTTCACATTTACCAGCAAGTC | Antisense | 695 |
| 244008_at | 485 | 285 | 144 | AATAGTGCTTATTTACATAGTCAAT | Antisense | 696 |
| 244008_at | 163 | 41 | 193 | ATCTTCGATCTGCCCAATATTTAAT | Antisense | 697 |
| 244008_at | 784 | 685 | 269 | GATATGCCTATGTTTCTTTAACTAT | Antisense | 698 |
| 244008_at | 58 | 1011 | 283 | TCTTTAACTATACAGCCTCTTTACA | Antisense | 699 |
| 244008_at | 121 | 721 | 338 | GTATTGCAACCTGCTATTTAGCCTT | Antisense | 700 |
| 244008_at | 473 | 17 | 353 | ATTTAGCCTTTGGTGCCTTAGAATT | Antisense | 701 |
| 244008_at | 872 | 1099 | 443 | TTTCTCTATTCACTTTTTATTGCTC | Antisense | 702 |
| 244008_at | 199 | 7 | 461 | ATTGCTCTTGCTTTCTATTGCTACT | Antisense | 703 |
| 244008_at | 591 | 509 | 480 | GCTACTAAAGCCTCTTTTATCCAGC | Antisense | 704 |
| 244008_at | 316 | 739 | 508 | GTAATAGTTCCAACATTGTAGCGAA | Antisense | 705 |

TABLE 15
250 PS gene list probe sequences

| | | | | | | |
|---|---|---|---|---|---|---|
| | | | Probe | | | |
| Probe Set | | | Interrogation | | Target | |
| Name | Probe.X | Probe.Y | Position | Probe Sequence | Strandedness | SEQ ID NO: |

| | | | | | | |
|---|---|---|---|---|---|---|
| 1553555_at | 371 | 809 | 416 | GGGTCTCCAGGAAGATCTCCCAGAT | Antisense | 706 |
| 1553555_at | 1082 | 911 | 446 | TGGGTATTATTCTTTGCTCCTGCAT | Antisense | 707 |
| 1553555_at | 364 | 1107 | 496 | TTTTTTAGCAGACCTCACTTCACAG | Antisense | 708 |
| 1553555_at | 628 | 1099 | 595 | TTTCTCTTCTGCTATCTGTGGTCTG | Antisense | 709 |
| 1553555_at | 405 | 85 | 695 | AGGTCTATACCAGAAACTCTCGTGA | Antisense | 710 |
| 1553555_at | 820 | 351 | 738 | CATTAAAGCCCTCAAGTCTCTTGTC | Antisense | 711 |
| 1553555_at | 1062 | 1137 | 769 | TTCTGCTTCTTTGTGATATCATCCT | Antisense | 712 |
| 1553555_at | 941 | 23 | 784 | ATATCATCCTGTGTTGCCTTCATCT | Antisense | 713 |
| 1553555_at | 1123 | 447 | 816 | CCTACTGATTCTGTGGCGCGACAAA | Antisense | 714 |
| 1553555_at | 5 | 839 | 863 | GGATAATGGCAGCTTGTCCCTCTGG | Antisense | 715 |
| 1553555_at | 940 | 141 | 930 | AGCTGTGATGACCATTCTGCTCTGG | Antisense | 716 |
| 1554999_at | 503 | 715 | 2949 | GTTTACCTAGTATTCATCACCCTGC | Antisense | 717 |
| 1554999_at | 888 | 1155 | 2974 | TTGTTTTCTCTGTGTTATGTCTTGG | Antisense | 718 |
| 1554999_at | 144 | 861 | 2997 | GGAAACCCTTCCATGTCAATGTAGG | Antisense | 719 |
| 1554999_at | 784 | 723 | 3027 | GTATCTTCTTTCAGCTGCTATACAG | Antisense | 720 |
| 1554999_at | 977 | 917 | 3062 | TGTGATTGGGCCATGTTTCTTTTTC | Antisense | 721 |
| 1554999_at | 765 | 261 | 3180 | AAGTCCCTTCTTCATATCAAACTCT | Antisense | 722 |
| 1554999_at | 90 | 1085 | 3224 | TATAGTTGTGGGTTGCTCTTGTAAA | Antisense | 723 |
| 1554999_at | 416 | 339 | 3249 | CAGGCTTTACTGACTGTGAAACTTC | Antisense | 724 |
| 1554999_at | 1085 | 1099 | 3291 | TTTCTACTCTGCACTTGATCTTAGC | Antisense | 725 |
| 1554999_at | 279 | 635 | 3325 | GAGAAGCGATTCTACCCTGGATAGT | Antisense | 726 |
| 1554999_at | 140 | 87 | 3377 | AGGTGTTTATTAGCCATTCTGAGTA | Antisense | 727 |
| 1556209_at | 465 | 105 | 253 | AGATTATGGCCTATTGAACAAAATG | Antisense | 728 |
| 1556209_at | 242 | 589 | 279 | GAAACTGGGAGTCCACAAAGACATA | Antisense | 729 |
| 1556209_at | 124 | 593 | 308 | GAAAGATGAAGTACCTCCTGACAGA | Antisense | 730 |
| 1556209_at | 357 | 565 | 315 | GAAGTACCTCCTGACAGAGCAAGAA | Antisense | 731 |
| 1556209_at | 202 | 859 | 361 | GGACAATGGCTATTTTATTCAAGAC | Antisense | 732 |
| 1556209_at | 375 | 637 | 403 | GAGACTAGGACTCAGTCTGAACTGA | Antisense | 733 |
| 1556209_at | 310 | 681 | 479 | GATCACACACCGTATGTGTTTGCTA | Antisense | 734 |
| 1556209_at | 337 | 929 | 495 | TGTTTGCTAACTGGCTTTACCCAAA | Antisense | 735 |
| 1556209_at | 464 | 861 | 612 | GGAAAAAGGCACTCAGGGCACACAG | Antisense | 736 |
| 1556209_at | 912 | 289 | 653 | AATTCTCTACAAACCTAGTTGCTAA | Antisense | 737 |
| 1556209_at | 869 | 1147 | 671 | TTGCTAAAACTACCTGTTGTAACCT | Antisense | 738 |
| 1556341_s_at | 335 | 75 | 2430 | AGGCTGTGCTGGACCTGGCTTCATT | Antisense | 739 |
| 1556341_s_at | 18 | 1131 | 2449 | TTCATTTCTGCTCCAGGATGGCAGG | Antisense | 740 |
| 1556341_s_at | 184 | 1013 | 2482 | TCTTCATGGCTGCATGACCAGCCTG | Antisense | 741 |
| 1556341_s_at | 974 | 1073 | 2529 | TAGGTGTACACTGGGAGCCGTGCCA | Antisense | 742 |
| 1556341_s_at | 411 | 165 | 2538 | ACTGGGAGCCGTGCCATGTTTCAGG | Antisense | 743 |
| 1556341_s_at | 331 | 973 | 2549 | TGCCATGTTTCAGGCCCTGTCCTGG | Antisense | 744 |
| 1556341_s_at | 763 | 809 | 2583 | GGGTGCGGGACCTTGGTCCTCCACA | Antisense | 745 |
| 1556341_s_at | 28 | 913 | 2627 | TGGGTTGAGATTCACATGTGCTCCT | Antisense | 746 |
| 1556341_s_at | 986 | 183 | 2640 | ACATGTGCTCCTTACGACCACGGGT | Antisense | 747 |
| 1556341_s_at | 864 | 159 | 2691 | ACCCTTCGCTGTGTGCATATTCGTG | Antisense | 748 |
| 1556341_s_at | 1153 | 765 | 2703 | GTGCATATTCGTGTGCTTATGAGTT | Antisense | 749 |
| 1557458_s_at | 863 | 159 | 3982 | ACCCTCTCCTTGAGTTTCTGTGAAT | Antisense | 750 |
| 1557458_s_at | 792 | 397 | 4068 | CTGATGCTGGGAGCCAAATGCTGGT | Antisense | 751 |
| 1557458_s_at | 841 | 203 | 4083 | AAATGCTGGTGCTTTGAGAGTCCCG | Antisense | 752 |
| 1557458_s_at | 15 | 765 | 4091 | GTGCTTTGAGAGTCCCGGAGGCCCC | Antisense | 753 |
| 1557458_s_at | 808 | 419 | 4124 | CGCCCCGCTGGTGTGTATATGTGTG | Antisense | 754 |
| 1557458_s_at | 613 | 825 | 4188 | GGTCACACACAGATGGGTAAGCCCA | Antisense | 755 |
| 1557458_s_at | 494 | 739 | 4204 | GTAAGCCCACTGATCTACTTGTAGT | Antisense | 756 |
| 1557458_s_at | 1037 | 677 | 4215 | GATCTACTTGTAGTCACTCAGTGTA | Antisense | 757 |
| 1557458_s_at | 222 | 99 | 4226 | AGTCACTCAGTGTAATCATTAGGCT | Antisense | 758 |
| 1557458_s_at | 249 | 41 | 4240 | ATCATTAGGCTATCTTCAAGGAATC | Antisense | 759 |
| 1557458_s_at | 310 | 1033 | 4255 | TCAAGGAATCATTGTGCAGTCAAAA | Antisense | 760 |
| 1558111_at | 478 | 217 | 1248 | AAACAGTTGATGGTGCACTCGTAGC | Antisense | 761 |
| 1558111_at | 511 | 667 | 1256 | GATGGTGCACTCGTAGCTGCAAAAT | Antisense | 762 |
| 1558111_at | 592 | 885 | 1297 | GGCTGTGACATTGAGTGGTTACACA | Antisense | 763 |
| 1558111_at | 552 | 91 | 1310 | AGTGGTTACACAGAAGACCTGCTTC | Antisense | 764 |
| 1558111_at | 1042 | 587 | 1356 | GAAACATTACTATTCAGTTGCTATT | Antisense | 765 |
| 1558111_at | 628 | 1107 | 1402 | TTTTAGTTGCTGCTCACTTATGGCC | Antisense | 766 |
| 1558111_at | 121 | 1149 | 1408 | TTGCTGCTCACTTATGGCCACAGAA | Antisense | 767 |
| 1558111_at | 561 | 893 | 1422 | TGGCCACAGAACAGTCCCTTTTTGA | Antisense | 768 |
| 1558111_at | 32 | 29 | 1434 | AGTCCCTTTTTGAGTAAAGCAGCAA | Antisense | 769 |
| 1558111_at | 670 | 275 | 1549 | AATGAACTCATTACAGCAGCTAATC | Antisense | 770 |
| 1558111_at | 423 | 133 | 1563 | AGCAGCTAATCAATGCAAACTTTGT | Antisense | 771 |
| 1561226_at | 1147 | 43 | 3089 | ATCTCCCTCAGTGCCTGGCAATGTT | Antisense | 772 |
| 1561226_at | 1100 | 1021 | 3096 | TCAGTGCCTGGCAATGTTTGTGGTT | Antisense | 773 |
| 1561226_at | 1092 | 969 | 3100 | TGCCTGGCAATGTTTGTGGTTGGTT | Antisense | 774 |
| 1561226_at | 3 | 1091 | 3112 | TTTGTGGTTGGTTGAGTGAGTTAAC | Antisense | 775 |
| 1561226_at | 449 | 773 | 3127 | GTGAGTTAACAGGAGACCATCTTTT | Antisense | 776 |
| 1561226_at | 297 | 185 | 3135 | ACAGGAGACCATCTTTTGGCCTTTT | Antisense | 777 |
| 1561226_at | 1087 | 1099 | 3159 | TTTCTACCTCTGTTTTCTCTTACTA | Antisense | 778 |
| 1561226_at | 337 | 713 | 3170 | GTTTTCTCTTACTATACTTGCCTAC | Antisense | 779 |
| 1561226_at | 897 | 1135 | 3173 | TTCTCTTACTATACTTGCCTACATC | Antisense | 780 |
| 1561226_at | 924 | 363 | 3177 | CTTACTATACTTGCCTACATCTCAT | Antisense | 781 |
| 1561226_at | 647 | 1083 | 3182 | TATACTTGCCTACATCTCATCTTCT | Antisense | 782 |
| 1562031_at | 1147 | 61 | 1892 | ATGTTCACTGTATGTGCCAAGCCTA | Antisense | 783 |
| 1562031_at | 289 | 165 | 1898 | ACTGTATGTGCCAAGCCTAATATGA | Antisense | 784 |
| 1562031_at | 811 | 761 | 1905 | GTGCCAAGCCTAATATGAGAGCTAT | Antisense | 785 |
| 1562031_at | 374 | 1103 | 1933 | TTATAGAGTTTATGCTACAGCCCTA | Antisense | 786 |
| 1562031_at | 1143 | 113 | 1937 | AGAGTTTATGCTACAGCCCTACCTT | Antisense | 787 |
| 1562031_at | 529 | 189 | 1949 | ACAGCCCTACCTTCAGGAAACTTAT | Antisense | 788 |
| 1562031_at | 743 | 77 | 1963 | AGGAAACTTATCTACTGGACAAACA | Antisense | 789 |
| 1562031_at | 377 | 179 | 2022 | ACATTGTAATTATCTAGCATAGGCA | Antisense | 790 |
| 1562031_at | 519 | 519 | 2038 | GCATAGGCAAATATAGACAGTAACA | Antisense | 791 |
| 1562031_at | 968 | 229 | 2059 | AACAGACAGGTTTACAATTATTAAG | Antisense | 792 |
| 1562031_at | 442 | 293 | 2073 | CAATTATTAAGAAAGGGCAGCCAGG | Antisense | 793 |
| 1562249_at | 1054 | 669 | 4456 | GATGACTATCTATGAACCTAAGGAG | Antisense | 794 |
| 1562249_at | 889 | 1033 | 4488 | TAATTTCTTTTGAGGGTTTGCATAA | Antisense | 795 |
| 1562249_at | 700 | 287 | 4690 | AATTTCTGGAATTTTCAATGAGCAA | Antisense | 796 |
| 1562249_at | 994 | 1043 | 4821 | TAAAATAGGCACATATCTGAAGATA | Antisense | 797 |
| 1562249_at | 1130 | 393 | 4837 | CTGAAGATAAAAGCCGAATTGACCC | Antisense | 798 |
| 1562249_at | 1134 | 11 | 4893 | ATTAATGGCTTTGAATGGCTATGAA | Antisense | 799 |
| 1562249_at | 1000 | 219 | 4917 | AAAAATAATATCTCTGCCCTCTTCA | Antisense | 800 |
| 1562249_at | 309 | 447 | 4934 | CCTCTTCACTACTCCCATGTAAAAT | Antisense | 801 |
| 1562249_at | 846 | 727 | 4969 | GTAGATTTTACCAACGTTTCAAGAG | Antisense | 802 |
| 1562249_at | 745 | 715 | 4984 | GTTTCAAGAGAGACATGATTCCCAA | Antisense | 803 |
| 1562249_at | 16 | 607 | 4995 | GACATGATTCCCAAATATTATGTAA | Antisense | 804 |
| 1563497_at | 251 | 1063 | 4804 | TACCATAGCAAGTTCTTATCCTTTG | Antisense | 805 |
| 1563497_at | 173 | 157 | 4861 | ACCTGTCACAGTAATATGCCAATTT | Antisense | 806 |
| 1563497_at | 943 | 467 | 4878 | GCCAATTTTTGGTGTCGCTTTAGTA | Antisense | 807 |
| 1563497_at | 834 | 749 | 4965 | GTCTTTCCTCAGTTGTCTCCTAAAA | Antisense | 808 |
| 1563497_at | 555 | 677 | 5022 | GATCGTCTTGTGCAGCCCTTTAAAA | Antisense | 809 |
| 1563497_at | 941 | 53 | 5048 | ATGCGTTTTGCATTCATGTCTTTGC | Antisense | 810 |
| 1563497_at | 920 | 365 | 5088 | CTTTCATTTTGGGAGTGACTGTCAT | Antisense | 811 |
| 1563497_at | 1079 | 189 | 5141 | ACACCAGGGCCAGATTGCGTGGGTT | Antisense | 812 |
| 1563497_at | 610 | 965 | 5156 | TGCGTGGGTTCTAGCTAGGGACCTA | Antisense | 813 |
| 1563497_at | 1053 | 141 | 5182 | AGCTGGTTACTTGATTTCTCTCTGT | Antisense | 814 |
| 1563497_at | 93 | 1137 | 5197 | TTCTCTCTGTTCCATTTACTATCTG | Antisense | 815 |
| 1565544_at | 778 | 729 | 1683 | GTAGAGTTCTCTAAAGGGTCTTGAT | Antisense | 816 |
| 1565544_at | 871 | 263 | 1696 | AAGGGTCTTGATATTAGTGGGACAT | Antisense | 817 |
| 1565544_at | 612 | 89 | 1711 | AGTGGGACATAATTAGTTCTAAACT | Antisense | 818 |
| 1565544_at | 704 | 95 | 1802 | AGTCACTTAACTATAGTCCAGAAAA | Antisense | 819 |
| 1565544_at | 891 | 825 | 1876 | GGTAAAATTCACAAAGTCTGCCACA | Antisense | 820 |
| 1565544_at | 205 | 97 | 1890 | AGTCTGCCACAAAGTCTGGCATCCA | Antisense | 821 |
| 1565544_at | 1138 | 257 | 1901 | AAGTCTGGCATCCAGTTGAGAACTA | Antisense | 822 |
| 1565544_at | 849 | 105 | 1914 | AGTTGAGAACTACCAGACATACAAA | Antisense | 823 |
| 1565544_at | 702 | 123 | 1971 | AGAAAAATCAATCATAACCACCCAG | Antisense | 824 |
| 1565544_at | 264 | 1017 | 1982 | TCATAACCACCCAGAACTAACACAG | Antisense | 825 |
| 1565544_at | 749 | 1051 | 2164 | TACACTGGATAGAATTAATGGCAAG | Antisense | 826 |
| 1568752_s_at | 370 | 107 | 4615 | AGATTAACATTGACAGTTCGACAAG | Antisense | 827 |
| 1568752_s_at | 224 | 703 | 4630 | GTTCGACAAGAGAGACTATCATCAG | Antisense | 828 |
| 1568752_s_at | 622 | 897 | 4717 | TGGAAAGGGATTCCTACCCCAGATT | Antisense | 829 |
| 1568752_s_at | 1092 | 689 | 4725 | GATTCCTACCCCAGATTTCTAAAGT | Antisense | 830 |
| 1568752_s_at | 318 | 735 | 4748 | GTCAGAAATGTACCAAAAACTTTTG | Antisense | 831 |
| 1568752_s_at | 806 | 175 | 4766 | ACTTTTGAAAACTATGCAGTCCAAC | Antisense | 832 |
| 1568752_s_at | 196 | 175 | 4776 | ACTATGCAGTCCAACAACAGTTTCT | Antisense | 833 |
| 1568752_s_at | 659 | 715 | 4795 | GTTTCTGACTACAACTCAAAAGTTT | Antisense | 834 |
| 1568752_s_at | 530 | 935 | 4853 | TGATCTTTTTATTTAGAAACCCACA | Antisense | 835 |
| 1568752_s_at | 795 | 589 | 4868 | GAAACCCACAAAATCAGAAACACAG | Antisense | 836 |
| 1568752_s_at | 892 | 733 | 4999 | GTACAGCAAGCCTATGTAGTTCAAT | Antisense | 837 |
| 200924_s_at | 738 | 189 | 1290 | ACAGCCTATGGAGGCTCCAGTCATG | Antisense | 838 |
| 200924_s_at | 629 | 883 | 1302 | GGCTCCAGTCATGCTGTGGGATGAG | Antisense | 839 |
| 200924_s_at | 828 | 403 | 1315 | CTGTGGGATGAGTCCAGCTTCCCTG | Antisense | 840 |
| 200924_s_at | 151 | 1141 | 1333 | TTCCCTGACATCCCAGGGGCTGTAA | Antisense | 841 |
| 200924_s_at | 301 | 815 | 1348 | GGGGCTGTAAGTGCCAACATGACTG | Antisense | 842 |
| 200924_s_at | 270 | 135 | 1433 | AGCGGAGTAAGGAGCGCTCCCTACT | Antisense | 843 |
| 200924_s_at | 251 | 991 | 1450 | TCCCTACTGCATGGGGACTTCCACG | Antisense | 844 |
| 200924_s_at | 1131 | 165 | 1514 | ACTGGGACCAGAATGAGCGTTTTCT | Antisense | 845 |
| 200924_s_at | 980 | 625 | 1528 | GAGCGTTTTCTGGTAGTGCTTAACT | Antisense | 846 |
| 200924_s_at | 834 | 1043 | 1548 | TAACTTTGGGGATGTGGGCCTCTCG | Antisense | 847 |
| 200924_s_at | 385 | 237 | 1691 | AACTGGAGCCTCACGAAGGGCTGCT | Antisense | 848 |
| 201008_s_at | 6 | 541 | 250 | GCAAAGGGGTTTCCTCGATTTGGAG | Antisense | 849 |
| 201008_s_at | 262 | 1147 | 286 | TTCCACCGTCATTTCTAACTCTTAA | Antisense | 850 |
| 201008_s_at | 782 | 213 | 310 | AAACCAACTCAGTTCCATCATGGTG | Antisense | 851 |
| 201008_s_at | 525 | 907 | 358 | TGAGGTGGTCTTTAACGACCCTGAA | Antisense | 852 |
| 201008_s_at | 587 | 563 | 437 | GAAGTTACTCGTGTCAAAGCCGTTA | Antisense | 853 |
| 201008_s_at | 1020 | 675 | 463 | GATCCTGGCTTGCGGAGTGGCTAAA | Antisense | 854 |
| 201008_s_at | 1135 | 903 | 494 | TGGATGCAGGGATCCCAGCAGTGCA | Antisense | 855 |
| 201008_s_at | 1003 | 409 | 528 | CGGAGTACCTGCGCTATGAAGACAC | Antisense | 856 |
| 201008_s_at | 1015 | 1049 | 623 | TACAAGTTCGGCTTTGAGCTTCCTC | Antisense | 857 |
| 201008_s_at | 3 | 437 | 722 | CCGAGCCAGCCAACTCAAGAGACAA | Antisense | 858 |
| 201008_s_at | 1015 | 815 | 772 | GGTGGATGTCAATACCCCTGATTTA | Antisense | 859 |
| 201010_s_at | 1118 | 775 | 2116 | GTGTTCTCCTACTGCAAATATTTTC | Antisense | 860 |
| 201010_s_at | 612 | 829 | 2153 | GGTTTTCTCTTCATGTAAGTCCTTG | Antisense | 861 |
| 201010_s_at | 776 | 931 | 2196 | TGTTCTTAGCACTTTAATTCCTGTC | Antisense | 862 |
| 201010_s_at | 968 | 499 | 2259 | GCTGAAACTGGTCTACTGTGTCTCT | Antisense | 863 |
| 201010_s_at | 909 | 593 | 2373 | GAAAGGGTTGCTGCTGTCAGCCTTG | Antisense | 864 |
| 201010_s_at | 538 | 301 | 2444 | CAACCCTGTGATCAGAACCTCCAAA | Antisense | 865 |
| 201010_s_at | 777 | 107 | 2540 | AGATAGGGATATTGGCCCCTCACTG | Antisense | 866 |
| 201010_s_at | 1097 | 313 | 2587 | CACTCTCAGCCATAGCACTTTGTTC | Antisense | 867 |
| 201010_s_at | 814 | 175 | 2603 | ACTTTGTTCACTGTCCTGTGTCAGA | Antisense | 868 |
| 201010_s_at | 668 | 917 | 2619 | TGTGTCAGAGCACTGAGCTCCACCC | Antisense | 869 |
| 201010_s_at | 799 | 139 | 2634 | AGCTCCACCCTTTTCTGAGAGTTAT | Antisense | 870 |
| 201087_at | 542 | 39 | 3029 | ATCAGGACTCTGACGTCTCTTTGGT | Antisense | 871 |
| 201087_at | 291 | 699 | 3057 | GTTGATTTACCTCTGGGCATATCCC | Antisense | 872 |
| 201087_at | 319 | 1029 | 3106 | TCACAGGTGGGATCGGCACTCAGGG | Antisense | 873 |
| 201087_at | 403 | 453 | 3240 | CCTTGGGCTCCTGATGACCAGAAAG | Antisense | 874 |
| 201087_at | 706 | 789 | 3265 | GGGCCACCGGGGTCTAATGGTGACA | Antisense | 875 |
| 201087_at | 193 | 271 | 3280 | AATGGTGACAGTCCAAACCACTCCA | Antisense | 876 |
| 201087_at | 918 | 479 | 3308 | GCCTCCTGGCAGAAGCCGAGTGTGC | Antisense | 877 |
| 201087_at | 175 | 967 | 3330 | TGCTGGGGTCTCCGAAGAGGGTCCC | Antisense | 878 |
| 201087_at | 260 | 443 | 3383 | CCTCCAAAGGTCTGATGTCTCCACT | Antisense | 879 |
| 201087_at | 9 | 475 | 3419 | GCCTTACCGCTCTGTTTATAGTGAC | Antisense | 880 |
| 201087_at | 114 | 1105 | 3433 | TTTATAGTGACCCACCCTAGATCTT | Antisense | 881 |
| 201115_at | 434 | 449 | 1082 | CCTACCAGGCCACCATTGATGGAGT | Antisense | 882 |
| 201115_at | 372 | 649 | 1103 | GAGTCAGATTTTTGGGGACATCAGG | Antisense | 883 |
| 201115_at | 816 | 799 | 1117 | GGGACATCAGGACAGAACGTGAGTG | Antisense | 884 |
| 201115_at | 1101 | 769 | 1139 | GTGACATTTTCCGATACAGCAGCAT | Antisense | 885 |
| 201115_at | 772 | 1141 | 1147 | TTCCGATACAGCAGCATGGAGGATC | Antisense | 886 |
| 201115_at | 712 | 49 | 1180 | ATCCTGGAGTGGACCCTGCGGGTCC | Antisense | 887 |
| 201115_at | 783 | 373 | 1251 | CTACAAAACTGACCCGTTCATCTTC | Antisense | 888 |
| 201115_at | 621 | 883 | 1321 | GGCTCCAAAATCATCCGAGGTCCTG | Antisense | 889 |
| 201115_at | 249 | 623 | 1348 | GACCAGACAGTGCTGTTGGTGACTG | Antisense | 890 |
| 201115_at | 53 | 513 | 1451 | GCTTCGGGGCAGAGGACGATGACCT | Antisense | 891 |
| 201115_at | 522 | 875 | 1458 | GGCAGAGGACGATGACCTGGGAGGC | Antisense | 892 |
| 201364_s_at | 63 | 183 | 370 | ACATCGTCCACTTCCAGTATGAGGT | Antisense | 893 |
| 201364_s_at | 885 | 561 | 404 | GAAGGTCTCTTCTTGGGATGCAGTC | Antisense | 894 |
| 201364_s_at | 515 | 769 | 534 | GTGAACTATGTCTTCATCTGCTTCA | Antisense | 895 |
| 201364_s_at | 720 | 89 | 562 | AGGGCCGAGAAGACAGAGCTCCACT | Antisense | 896 |
| 201364_s_at | 576 | 795 | 608 | GGGCTTTGAGATTGTACGTCCAGGC | Antisense | 897 |
| 201364_s_at | 99 | 427 | 645 | CCCTCTCGGCCAGATGTGATGTTCA | Antisense | 898 |
| 201364_s_at | 148 | 931 | 664 | TGTTCATGGTTTATCCCCTGGACCA | Antisense | 899 |
| 201364_s_at | 669 | 47 | 676 | ATCCCCTGGACCAGAACTTGTCCGA | Antisense | 900 |
| 201364_s_at | 618 | 673 | 722 | GATGCTTTACCCAAGAGCCACAGTG | Antisense | 901 |
| 201364_s_at | 290 | 883 | 788 | GGCTCCTCGCTGTCTAGGGAAGGAC | Antisense | 902 |
| 201364_s_at | 915 | 939 | 829 | TGAGGGTTGCCTATTGTGTTCTCGG | Antisense | 903 |
| 201870_at | 752 | 919 | 1290 | TGTCCTGCCCAAGTGGGCTGCAGAC | Antisense | 904 |
| 201870_at | 167 | 395 | 1307 | CTGCAGACTGAGTGCTGCCCTTGTA | Antisense | 905 |
| 201870_at | 181 | 421 | 1344 | CCCAACTCACTGCAGTTCATCTGAA | Antisense | 906 |
| 201870_at | 434 | 1131 | 1359 | TTCATCTGAACAACCTGAGCTCCTG | Antisense | 907 |
| 201870_at | 908 | 153 | 1409 | ACCTAAGGCCCTGATCCAAAGCAGC | Antisense | 908 |
| 201870_at | 1142 | 479 | 1432 | GCCTGTTGAGCTGGTTCTCCAGGGC | Antisense | 909 |
| 201870_at | 1083 | 357 | 1500 | CTTGCACAGTCTCCTATGTCTGAGC | Antisense | 910 |
| 201870_at | 466 | 627 | 1567 | GAGCCCTGACCCTTGAATGGTTGTC | Antisense | 911 |
| 201870_at | 720 | 953 | 1580 | TGAATGGTTGTCCTTGACTCTGTGC | Antisense | 912 |
| 201870_at | 612 | 627 | 1642 | GAGCCCAGTGATTGTGGCTGCTCCT | Antisense | 913 |
| 201870_at | 613 | 385 | 1756 | CTCCGGGCTTCATAGTTCTTTGTAA | Antisense | 914 |
| 201909_at | 474 | 605 | 306 | GACAGGTGAACATTTCCGCCTGGTC | Antisense | 915 |
| 201909_at | 965 | 1077 | 331 | TATGACACCAAGGGCCGTTTTGCTG | Antisense | 916 |
| 201909_at | 628 | 965 | 351 | TGCTGTTCACCGCATCACAGTGGAA | Antisense | 917 |
| 201909_at | 139 | 771 | 442 | GTGACTCATGATGCTCGAACCATCC | Antisense | 918 |
| 201909_at | 383 | 459 | 472 | CCAGATCCTGTCATCAAGGTGAACG | Antisense | 919 |
| 201909_at | 134 | 805 | 570 | GGTGATTGGTGGAGCCAACCTCGGT | Antisense | 920 |
| 201909_at | 543 | 155 | 587 | ACCTCGGTCGTGTTGGTGTGATCAC | Antisense | 921 |
| 201909_at | 156 | 607 | 623 | GACATCCTGGTTCTTTTGATGTGGT | Antisense | 922 |
| 201909_at | 440 | 1151 | 726 | TTGGATTTCCCTGCCCAGGGGAAAG | Antisense | 923 |
| 201909_at | 830 | 795 | 750 | GGGCATTCGACTTACTGTTGCTGAA | Antisense | 924 |
| 201909_at | 904 | 643 | 779 | GAGATAAGAGGCTGGCCACCAAACA | Antisense | 925 |
| 201938_at | 883 | 215 | 1154 | AAACTGTAGTTTGCCTCCAAGACAC | Antisense | 926 |
| 201938_at | 302 | 449 | 1188 | CCTTTAATCTTCTCTTTTGTATACA | Antisense | 927 |
| 201938_at | 230 | 1121 | 1217 | TTACCCATGGTGTTCTTTGTTCCTT | Antisense | 928 |
| 201938_at | 51 | 703 | 1235 | GTTCCTTTTCATAAGCTAATACCAC | Antisense | 929 |
| 201938_at | 238 | 577 | 1277 | GAACGCATATTGACAGCACGCTTTA | Antisense | 930 |
| 201938_at | 639 | 537 | 1292 | GCACGCTTTACTTAGTAGCCGGTTC | Antisense | 931 |
| 201938_at | 376 | 397 | 1311 | CGGTTCCCATTTGCCATACAATGTA | Antisense | 932 |
| 201938_at | 491 | 327 | 1429 | CAGAAGAAAGAGCAACCCACCAAAC | Antisense | 933 |
| 201938_at | 344 | 431 | 1465 | CCCCGAACACTTTCATACTAAGACT | Antisense | 934 |
| 201938_at | 66 | 729 | 1494 | GTAGATCTCAGTTCTGCGTTTATTG | Antisense | 935 |
| 201938_at | 1059 | 221 | 1552 | AAAACTGTTTGCATCTTTGTATGTA | Antisense | 936 |
| 201981_at | 352 | 187 | 9437 | ACAGATTGTTCTCAAGAGGGCCATC | Antisense | 937 |
| 201981_at | 858 | 535 | 9491 | GCACCAACAACTCAACATGGTCATC | Antisense | 938 |
| 201981_at | 846 | 905 | 9528 | TGGTTTTTCCAGCTAGCAGTACTCC | Antisense | 939 |
| 201981_at | 191 | 403 | 9562 | CTGTGACTGGGCAGTGCTTTTCTCT | Antisense | 940 |
| 201981_at | 662 | 991 | 9588 | TCCCATGTCTAGCCTCCAAAAGTTA | Antisense | 941 |
| 201981_at | 732 | 583 | 9616 | GAAAATTAGTCAACTGCACGTGGAA | Antisense | 942 |
| 201981_at | 316 | 465 | 9649 | CCACTTTGGGGATCTCTTTATTTCT | Antisense | 943 |
| 201981_at | 1007 | 639 | 9740 | GAGAGAAGGGTTGAGTGTGGCATAC | Antisense | 944 |
| 201981_at | 1079 | 91 | 9753 | AGTGTGGCATACTTTCTGAAACCTG | Antisense | 945 |
| 201981_at | 836 | 589 | 9770 | GAAACCTGGAGCTGGGAATTGCGGA | Antisense | 946 |
| 201981_at | 696 | 823 | 9803 | GGTCTAGACTAGTTACATCACATAG | Antisense | 947 |
| 202290_at | 1042 | 87 | 267 | AGGGCTCATCGACATCGAGAACCCC | Antisense | 948 |
| 202290_at | 470 | 825 | 318 | GGTCACACAACTGGATCTGGACGGG | Antisense | 949 |
| 202290_at | 672 | 839 | 336 | GGACGGGCCAAAGGAGCTTTCGAGG | Antisense | 950 |
| 202290_at | 567 | 51 | 412 | ATGCACTTGGCCGGGAAGACAGAGC | Antisense | 951 |
| 202290_at | 1035 | 885 | 458 | GGCTGGCCATCATCCGGAAACAGCG | Antisense | 952 |
| 202290_at | 550 | 659 | 484 | GAGGAGGCTGCCCGGAAGAAGGAAG | Antisense | 953 |
| 202290_at | 101 | 211 | 523 | AAAGACGATGCCACATTGTCAGGAA | Antisense | 954 |
| 202290_at | 907 | 861 | 544 | GGAAAACGAATGCAGTCACTCTCCC | Antisense | 955 |
| 202290_at | 601 | 529 | 555 | GCAGTCACTCTCCCTGAATAAGTAA | Antisense | 956 |
| 202290_at | 637 | 1039 | 573 | TAAGTAACTGCGACCCGTGGGAGGA | Antisense | 957 |
| 202290_at | 105 | 813 | 764 | GGGGTAGCTGCTATCTTTGAGACAG | Antisense | 958 |
| 202578_s_at | 668 | 405 | 1831 | CTGGTCACAGTGGTAGTCGCTGGCC | Antisense | 959 |
| 202578_s_at | 612 | 427 | 1864 | CCCTCCTGATTTTGGCTAGGCATCG | Antisense | 960 |
| 202578_s_at | 157 | 371 | 1879 | CTAGGCATCGTGGAACCAGCTCCAG | Antisense | 961 |
| 202578_s_at | 173 | 767 | 1924 | GTGCAGGTTGTGCGGAAGAGGCTGA | Antisense | 962 |
| 202578_s_at | 1161 | 421 | 1967 | CCCACCGCTGTGCATCGAATGAGGG | Antisense | 963 |
| 202578_s_at | 104 | 71 | 2037 | ATGGACCCTTCACAAGTGTTTCTCC | Antisense | 964 |
| 202578_s_at | 828 | 359 | 2077 | CTTCCTTCTCCTGAAATCCAGAGAC | Antisense | 965 |
| 202578_s_at | 1003 | 277 | 2091 | AATCCAGAGACTTTCTTGCTCATCC | Antisense | 966 |
| 202578_s_at | 520 | 591 | 2147 | GAAAGCAGACTTGCATATCCCTGAT | Antisense | 967 |
| 202578_s_at | 306 | 1081 | 2162 | TATCCCTGATGAGCAGCTCGGCTAC | Antisense | 968 |
| 202578_s_at | 560 | 533 | 2174 | GCAGCTCGGCTACTTTTGTAAACTT | Antisense | 969 |
| 202580_x_at | 886 | 1033 | 2815 | TCAATTGACTTCTGTTCCTTGCTTT | Antisense | 970 |
| 202580_x_at | 868 | 253 | 2857 | AAGACCTGCAGTGCACGGTTTCTTC | Antisense | 971 |
| 202580_x_at | 1154 | 409 | 2872 | CGGTTTCTTCCAGGCTGAGGTACCT | Antisense | 972 |
| 202580_x_at | 248 | 657 | 2888 | GAGGTACCTGGATCTTGGGTTCTTC | Antisense | 973 |
| 202580_x_at | 928 | 911 | 2903 | TGGGTTCTTCACTGCAGGGACCCAG | Antisense | 974 |
| 202580_x_at | 913 | 263 | 2930 | AAGTGGATCTGCTTGCCAGAGTCCT | Antisense | 975 |
| 202580_x_at | 416 | 929 | 2981 | TGTTTCCAAGTCAGCTTTCCTGCAA | Antisense | 976 |
| 202580_x_at | 541 | 763 | 3094 | GTGCCCAGATGTGCGCTATTAGATG | Antisense | 977 |
| 202580_x_at | 716 | 669 | 3115 | GATGTTTCTCTGATAATGTCCCCAA | Antisense | 978 |
| 202580_x_at | 526 | 1147 | 3227 | TTGCCCCTCAGCTTTGCAAAGAGCC | Antisense | 979 |
| 202580_x_at | 463 | 461 | 3262 | CCAGCTGACCGCATGGGTGTGAGCC | Antisense | 980 |
| 202727_s_at | 125 | 247 | 1661 | AAGCGTAAAGAGGATGTGTGGCATT | Antisense | 981 |
| 202727_s_at | 763 | 781 | 1676 | GTGTGGCATTTTCACTTTTGGCTTG | Antisense | 982 |
| 202727_s_at | 91 | 879 | 1680 | GGCATTTTCACTTTTGGCTTGTAAA | Antisense | 983 |
| 202727_s_at | 390 | 249 | 1736 | AAGCATTGTAACTTATGAACCTTTA | Antisense | 984 |
| 202727_s_at | 165 | 177 | 1746 | ACTTATGAACCTTTACATCCAGATA | Antisense | 985 |
| 202727_s_at | 2 | 321 | 1761 | CATCCAGATAGGTTACCAGTAACGG | Antisense | 986 |
| 202727_s_at | 820 | 1077 | 1790 | TATCCAGTACTCCTGGTTCCTAGGT | Antisense | 987 |
| 202727_s_at | 668 | 733 | 1796 | GTACTCCTGGTTCCTAGGTGAGCAG | Antisense | 988 |
| 202727_s_at | 858 | 1073 | 1810 | TAGGTGAGCAGGTGATGCCCCAGGG | Antisense | 989 |
| 202727_s_at | 915 | 1099 | 1863 | TTTCTCTGCCTTGGTATAGCATATG | Antisense | 990 |
| 202727_s_at | 832 | 739 | 1894 | GTAAGTTTATGCATACAGTAATTTT | Antisense | 991 |
| 202948_at | 868 | 873 | 4351 | GGCAGCTGGAATTTAAGGAGGGACA | Antisense | 992 |
| 202948_at | 925 | 261 | 4419 | AAGTGCAAAGTTATTCCCCATCTTC | Antisense | 993 |
| 202948_at | 49 | 455 | 4436 | CCATCTTCCAAGGGTTGAATTCTGG | Antisense | 994 |
| 202948_at | 511 | 569 | 4463 | GAAGAAGACACATTCCTAGTTCCCC | Antisense | 995 |
| 202948_at | 84 | 429 | 4485 | CCCGTGAACTTCCTTTGACTTATTG | Antisense | 996 |
| 202948_at | 370 | 1151 | 4499 | TTGACTTATTGTCCCCACTAAAACA | Antisense | 997 |
| 202948_at | 252 | 1101 | 4562 | TTTCTTGCAGTTTTTTTATGGCATT | Antisense | 998 |
| 202948_at | 941 | 209 | 4592 | AAAGATGCCCTAAGTGTTGAAGAAG | Antisense | 999 |
| 202948_at | 737 | 211 | 4835 | AAAGCACCAAATTCATGTACAGCAT | Antisense | 1000 |
| 202948_at | 1035 | 343 | 4848 | CATGTACAGCATGCATCACGGATCA | Antisense | 1001 |
| 202948_at | 30 | 523 | 4860 | GCATCACGGATCAATAGACTGTACT | Antisense | 1002 |
| 203071_at | 600 | 485 | 2404 | GCCCCTGGAGTCGCGGAGAAAGGGC | Antisense | 1003 |
| 203071_at | 1125 | 239 | 2485 | AACGCACTGGTGACCAGACTGTCCC | Antisense | 1004 |
| 203071_at | 78 | 463 | 2509 | CCACGCCGGGAACCAAGCAGGAGAC | Antisense | 1005 |
| 203071_at | 761 | 641 | 2541 | GAGAGAGGAGCCAGACAGACCCTGA | Antisense | 1006 |
| 203071_at | 1040 | 481 | 2584 | GCCGGGCACATTGGGGGTCACCGGC | Antisense | 1007 |
| 203071_at | 947 | 435 | 2608 | CCGATGGAGACACCAACCGACAGGC | Antisense | 1008 |
| 203071_at | 183 | 535 | 2644 | GCAGCTGCGCGGGCTTATTTATTAA | Antisense | 1009 |
| 203071_at | 723 | 681 | 2672 | GATAACCCTTGAATGTAGCAGCCCC | Antisense | 1010 |
| 203071_at | 1112 | 309 | 2707 | CACAGGTCGGGCGCAGGATTCAGCC | Antisense | 1011 |
| 203071_at | 757 | 81 | 2721 | AGGATTCAGCCGGAGGGAAGGGACG | Antisense | 1012 |
| 203071_at | 143 | 463 | 2817 | CCACCTCCTTGGGTAGTGAGCAGTG | Antisense | 1013 |
| 203577_at | 773 | 11 | 1074 | ATTACCGACTGTATGCCTACACGGA | Antisense | 1014 |
| 203577_at | 182 | 1051 | 1091 | TACACGGAGTCGGAGCTGCAGATTG | Antisense | 1015 |
| 203577_at | 448 | 1143 | 1151 | TTCCCCAACATGGTGGTGGCGCAGG | Antisense | 1016 |
| 203577_at | 574 | 533 | 1192 | GCAGCAGGCAATCGCCAGTGGCATC | Antisense | 1017 |
| 203577_at | 1036 | 321 | 1218 | CAGCCCAGCAGATAATCCATTTCCT | Antisense | 1018 |
| 203577_at | 450 | 327 | 1336 | CAGACTCCGGTTCACTGAGGGTGTC | Antisense | 1019 |
| 203577_at | 219 | 655 | 1352 | GAGGGTGTCCTGTATAACCAGTTCC | Antisense | 1020 |
| 203577_at | 559 | 243 | 1367 | AACCAGTTCCTGTCGCAAGTGGACT | Antisense | 1021 |
| 203577_at | 598 | 1139 | 1436 | TTCGAGAACTCGGCCAAGCGGCTCA | Antisense | 1022 |
| 203577_at | 681 | 871 | 1479 | GGCACAGCGACGTCAAGCGCTTTTG | Antisense | 1023 |
| 203577_at | 696 | 505 | 1521 | GCTCCTGAGAGCGCGGGACTTGGAC | Antisense | 1024 |
| 203642_s_at | 174 | 313 | 4261 | CACTTAGTGTGAAAGACTTGCCTTG | Antisense | 1025 |
| 203642_s_at | 75 | 609 | 4275 | GACTTGCCTTGTGTATTCTTTATGT | Antisense | 1026 |
| 203642_s_at | 781 | 315 | 4310 | CACTGTCAATTTTATGGGAAGCTCA | Antisense | 1027 |
| 203642_s_at | 744 | 947 | 4431 | TGACATTGTCACACAGAAATTACAC | Antisense | 1028 |
| 203642_s_at | 791 | 179 | 4454 | ACATTTTATGTGCCAGAAGCCTTAA | Antisense | 1029 |
| 203642_s_at | 231 | 921 | 4462 | TGTGCCAGAAGCCTTAAACATCTTT | Antisense | 1030 |
| 203642_s_at | 1 | 543 | 4706 | GCAAAACAATGCCCATTTGTCGGCT | Antisense | 1031 |
| 203642_s_at | 443 | 277 | 4713 | AATGCCCATTTGTCGGCTTACAGCT | Antisense | 1032 |
| 203642_s_at | 23 | 751 | 4746 | GTCTAGAGCCTGTTGGTGTTCTGTC | Antisense | 1033 |
| 203642_s_at | 220 | 807 | 4760 | GGTGTTCTGTCCCTACTTTAAGAAT | Antisense | 1034 |
| 203642_s_at | 593 | 287 | 4782 | AATTTAATTGCTCACTTATTCTGAA | Antisense | 1035 |
| 203679_at | 457 | 491 | 747 | GCGCTTCTTCCAGGACAAGCGCCCG | Antisense | 1036 |
| 203679_at | 269 | 519 | 834 | GCATATGAGACTTGGGGGTCCCTCC | Antisense | 1037 |
| 203679_at | 1076 | 211 | 879 | AAACGGGAGTGTGCAGTCAGGGCCT | Antisense | 1038 |
| 203679_at | 1101 | 1065 | 963 | TAGAAGGGACGGCTCAGTGGCTGCA | Antisense | 1039 |
| 203679_at | 417 | 617 | 990 | GACGGTCCTGGAAATCTCACATGGT | Antisense | 1040 |
| 203679_at | 1040 | 913 | 1014 | TGGGCACTGCAGCGTTGGAACGTGA | Antisense | 1041 |
| 203679_at | 752 | 899 | 1029 | TGGAACGTGAGCCTCGGATTTCCTG | Antisense | 1042 |
| 203679_at | 674 | 485 | 1054 | GCCCCTCTACTGTAAATGTGCCTTA | Antisense | 1043 |
| 203679_at | 1068 | 887 | 1143 | GGCCCTCCAAGCCAGGTTGAGGTCT | Antisense | 1044 |
| 203679_at | 717 | 889 | 1229 | GGCCCCACACAGGCATGAGGGATGG | Antisense | 1045 |
| 203679_at | 1153 | 891 | 1251 | TGGCCCGGCCAAAGTCTAGGCAGAA | Antisense | 1046 |
| 203756_at | 1068 | 909 | 5721 | TGGGGACCTGTCAGGCTTTGTCATT | Antisense | 1047 |
| 203756_at | 773 | 1097 | 5744 | TTTCCCAGTTTGTTGGTGGTGCCTT | Antisense | 1048 |
| 203756_at | 398 | 761 | 5762 | GTGCCTTTAGTGGTTCCCTAATTTG | Antisense | 1049 |
| 203756_at | 927 | 1155 | 5784 | TTGGGAACACTGATGGGGCCTTGGA | Antisense | 1050 |
| 203756_at | 462 | 957 | 5894 | TGCACTGGCACATATGGTCACCTTG | Antisense | 1051 |
| 203756_at | 899 | 903 | 5917 | TGGAGGGCAGACCTAGGAGCCCCTC | Antisense | 1052 |
| 203756_at | 754 | 79 | 5995 | AGGACCCCCCAAAGCTGAGGGGCTG | Antisense | 1053 |
| 203756_at | 741 | 813 | 6013 | GGGGCTGAATGTAGCCTTTTCAACA | Antisense | 1054 |
| 203756_at | 665 | 269 | 6041 | AAGGCTCCCACTTGAGAGCAGCCTC | Antisense | 1055 |
| 203756_at | 149 | 445 | 6111 | CCTCGCTTCTTCAGCTAAAACTCCA | Antisense | 1056 |
| 203756_at | 118 | 785 | 6197 | GTGGGTCATTGCGGTCTTAGATTAT | Antisense | 1057 |
| 203793_x_at | 373 | 1075 | 1662 | TAGGCATGATTCCAACTGGCTGCAT | Antisense | 1058 |
| 203793_x_at | 746 | 431 | 1771 | CCCCGCGTTGTTGAATGTCCAGAAA | Antisense | 1059 |
| 203793_x_at | 971 | 275 | 1820 | AATGCAGTTTATCCCTGGTTCTGAG | Antisense | 1060 |
| 203793_x_at | 650 | 1069 | 1915 | TAGATAGTTCTGCCTCTTTTTTTTT | Antisense | 1061 |
| 203793_x_at | 1045 | 781 | 1950 | GTGTGTGGCCTTTGCATCATTTATC | Antisense | 1062 |
| 203793_x_at | 573 | 657 | 2002 | GAGGTCTCAGCTCTTGGAGGAGGGC | Antisense | 1063 |
| 203793_x_at | 673 | 241 | 2082 | AACCCTCACTAGCAGGACTGGTGGT | Antisense | 1064 |
| 203793_x_at | 829 | 751 | 2110 | GTCTCACCTGGGGCCTAGAGTGGAA | Antisense | 1065 |
| 203793_x_at | 712 | 809 | 2139 | GGGTGGGTTAACCTCACACAAGCAC | Antisense | 1066 |
| 203793_x_at | 41 | 299 | 2157 | CAAGCACAGATCCCAGACTTTGCCA | Antisense | 1067 |
| 203793_x_at | 989 | 75 | 2183 | AGGCAAACAGGGAATTCCGCCGATA | Antisense | 1068 |
| 203967_at | 467 | 1035 | 2066 | TAATATCTTTGGGTCTTACTATTTT | Antisense | 1069 |
| 203967_at | 1161 | 303 | 2095 | CATAAAAGTGACCAGGTAGACCCTT | Antisense | 1070 |
| 203967_at | 1056 | 1033 | 2122 | TAATTACATTCACTACTTCTACCAC | Antisense | 1071 |
| 203967_at | 584 | 11 | 2129 | ATTCACTACTTCTACCACTTGTGTA | Antisense | 1072 |
| 203967_at | 778 | 149 | 2142 | ACCACTTGTGTATCTCTAGCCAATG | Antisense | 1073 |
| 203967_at | 408 | 723 | 2151 | GTATCTCTAGCCAATGTGCTTGCAA | Antisense | 1074 |
| 203967_at | 922 | 765 | 2166 | GTGCTTGCAAGTGTACAGATCTGTG | Antisense | 1075 |
| 203967_at | 416 | 67 | 2198 | ATGTGTGTATATTTACCTCTTCGTT | Antisense | 1076 |
| 203967_at | 1043 | 1103 | 2209 | TTTACCTCTTCGTTTGCTCAAACAT | Antisense | 1077 |
| 203967_at | 600 | 933 | 2268 | TGTTGTTTTTGAGGCGCGTCTCACC | Antisense | 1078 |
| 203967_at | 950 | 345 | 2549 | CATGCTCAGCCATTAAGGTATTTTG | Antisense | 1079 |
| 203992_s_at | 114 | 77 | 4286 | AGGAAATAACCCAGTTCTGCACCAC | Antisense | 1080 |
| 203992_s_at | 245 | 463 | 4307 | CCACTGGTTTTTGTAGCTATCTCGT | Antisense | 1081 |
| 203992_s_at | 1100 | 367 | 4323 | CTATCTCGTAAGGCTGCTGGCTGAA | Antisense | 1082 |
| 203992_s_at | 261 | 397 | 4343 | CTGAAAACTGTGTCTATGCAACCTT | Antisense | 1083 |
| 203992_s_at | 578 | 543 | 4360 | GCAACCTTCCAAGTGCGGAGTGTCA | Antisense | 1084 |
| 203992_s_at | 92 | 495 | 4374 | GCGGAGTGTCAACCAACTGGACGGG | Antisense | 1085 |
| 203992_s_at | 443 | 411 | 4395 | CGGGAGAGAGTACTGCTCCTACTCC | Antisense | 1086 |
| 203992_s_at | 638 | 373 | 4413 | CTACTCCAGGACTCTCACAAAGCTG | Antisense | 1087 |
| 203992_s_at | 124 | 239 | 4494 | AACTGGCAACATCTTACAGACTACT | Antisense | 1088 |
| 203992_s_at | 416 | 611 | 4512 | GACTACTGACTTGAAGACAACCTCT | Antisense | 1089 |
| 203992_s_at | 711 | 59 | 4754 | ATGATGGTTACACTTTTGGTTCCTA | Antisense | 1090 |
| 204078_at | 1122 | 233 | 2016 | AACTTAAGATGGACACAGCTGACTG | Antisense | 1091 |
| 204078_at | 339 | 111 | 2022 | AGATGGACACAGCTGACTGGACCCC | Antisense | 1092 |
| 204078_at | 40 | 313 | 2091 | CACTTCTATGTCTCTGGACCACAGG | Antisense | 1093 |
| 204078_at | 125 | 871 | 2137 | GGCAAGTGGGCTGATGGGGTCCGCC | Antisense | 1094 |
| 204078_at | 895 | 751 | 2192 | GTCTCCTGGACAAGGAGTTTCCAGC | Antisense | 1095 |
| 204078_at | 470 | 439 | 2196 | CCTGGACAAGGAGTTTCCAGCTGCT | Antisense | 1096 |
| 204078_at | 460 | 885 | 2221 | GGCTGGAGTCTCAGGCCAAATTGCA | Antisense | 1097 |
| 204078_at | 55 | 73 | 2233 | AGGCCAAATTGCAGAGGGTCCTCCA | Antisense | 1098 |
| 204078_at | 1110 | 337 | 2256 | CAGGGTCCTGAAGAGCACTGGACTA | Antisense | 1099 |
| 204078_at | 1062 | 441 | 2262 | CCTGAAGAGCACTGGACTAAGAGTC | Antisense | 1100 |
| 204078_at | 250 | 173 | 2277 | ACTAAGAGTCTAGTGGTTCCAGGGC | Antisense | 1101 |
| 204233_s_at | 172 | 543 | 1157 | GCAAACATCCGGAAGTATCCCACCA | Antisense | 1102 |
| 204233_s_at | 743 | 1079 | 1172 | TATCCCACCAAGAAACAACAGCTCC | Antisense | 1103 |
| 204233_s_at | 950 | 1085 | 1201 | TATTTCCAGTTACTTGCCTGCATTC | Antisense | 1104 |
| 204233_s_at | 431 | 1035 | 1294 | TAATAGGTTTGCCCTTGCATCTCAT | Antisense | 1105 |
| 204233_s_at | 378 | 801 | 1327 | GGGACTGTGGTCCATTGTACAAGCC | Antisense | 1106 |
| 204233_s_at | 60 | 549 | 1367 | GAATTTGGGTACATGGACTACGCCC | Antisense | 1107 |
| 204233_s_at | 307 | 857 | 1381 | GGACTACGCCCAAGCAAGGTTTGAT | Antisense | 1108 |
| 204233_s_at | 803 | 565 | 1426 | GAAGCTTGGGGTGTGACTGTGGGGA | Antisense | 1109 |
| 204233_s_at | 152 | 155 | 1462 | ACCTCATCACTGGACTGCATGGGGA | Antisense | 1110 |
| 204233_s_at | 742 | 789 | 1530 | GTGGCAGGAGGCTTTGGGTGGCTCA | Antisense | 1111 |
| 204233_s_at | 936 | 1091 | 1542 | TTTGGGTGGCTCACTACTGAACACA | Antisense | 1112 |
| 204409_s_at | 697 | 83 | 224 | AGGTTATGCCATATCAGAGGGAAAT | Antisense | 1113 |
| 204409_s_at | 973 | 11 | 281 | ATTATATTGGTTGGTCTACGGGACT | Antisense | 1114 |
| 204409_s_at | 1058 | 819 | 293 | GGTCTACGGGACTATCAGGATAACA | Antisense | 1115 |
| 204409_s_at | 735 | 511 | 353 | GCTAGAAGCCTGAAGGCATATGGCG | Antisense | 1116 |
| 204409_s_at | 929 | 671 | 434 | GATGAAATCCAGTTTGACGATATTG | Antisense | 1117 |
| 204409_s_at | 377 | 93 | 500 | AGTGTTTTTACATGACAAGTTCTCT | Antisense | 1118 |
| 204409_s_at | 183 | 659 | 525 | GAGGATGGTTCTACAGTTGGGATTT | Antisense | 1119 |
| 204409_s_at | 129 | 1159 | 541 | TTGGGATTTTGGCCATCATCAACCA | Antisense | 1120 |
| 204409_s_at | 637 | 197 | 573 | AAATTCATTTAGTGTGTAGTTTCTG | Antisense | 1121 |
| 204409_s_at | 512 | 223 | 719 | AAAAGACCATAATATTTGCTGTTAA | Antisense | 1122 |
| 204409_s_at | 983 | 281 | 748 | AATAAACGAGTGCCTTTCCTACTGT | Antisense | 1123 |
| 204410_at | 165 | 449 | 849 | CCTTTCCTACTGTGATAACGTCAAG | Antisense | 1124 |
| 204410_at | 114 | 553 | 888 | GAATACATTTCTGCCTGATAATCAT | Antisense | 1125 |
| 204410_at | 551 | 971 | 899 | TGCCTGATAATCATGCTGAGTTCTA | Antisense | 1126 |
| 204410_at | 537 | 941 | 915 | TGAGTTCTAATAAGCCCTACTTCCA | Antisense | 1127 |
| 204410_at | 443 | 373 | 931 | CTACTTCCACCTAATCTGTTTACAG | Antisense | 1128 |
| 204410_at | 865 | 715 | 967 | GTTTCAGTTACTTAGATGGTCTCAT | Antisense | 1129 |
| 204410_at | 346 | 667 | 981 | GATGGTCTCATAAGGTTTCTGATAC | Antisense | 1130 |
| 204410_at | 835 | 1017 | 1115 | TCATCCTTTGTGCCTCGGTTATTAA | Antisense | 1131 |
| 204410_at | 807 | 561 | 1223 | GAAACCGAACAATAAGTGGCAACCA | Antisense | 1132 |
| 204410_at | 95 | 859 | 1262 | GGAAATACTGGGGTGCCATTTTGTT | Antisense | 1133 |
| 204410_at | 463 | 1155 | 1302 | TTGTAATCCACTGTTTTGGCTTTCA | Antisense | 1134 |
| 204434_at | 1148 | 279 | 3403 | AATCTTCTATGGCTCTCTTTTTAAT | Antisense | 1135 |
| 204434_at | 981 | 117 | 3543 | AGACCAGCCTAGCAGGGCATTCATC | Antisense | 1136 |
| 204434_at | 1026 | 877 | 3558 | GGCATTCATCTTTGATGTTCTTCCT | Antisense | 1137 |
| 204434_at | 665 | 1023 | 3592 | TCAGAATCGGCAATGCCTGGCCCAC | Antisense | 1138 |
| 204434_at | 94 | 1071 | 3637 | TAGTTTTCCATTTCCAGATTGCGCC | Antisense | 1139 |
| 204434_at | 1150 | 233 | 3677 | AACTCTTTCTAACCAACCGTGATGC | Antisense | 1140 |
| 204434_at | 290 | 51 | 3698 | ATGCCACGTAACTTACCCTATGGAC | Antisense | 1141 |
| 204434_at | 141 | 95 | 3758 | AGTCCAGTGTACCTGTCATCACGGA | Antisense | 1142 |
| 204434_at | 841 | 349 | 3774 | CATCACGGACGCACTGTGGTCACAG | Antisense | 1143 |
| 204434_at | 992 | 283 | 3804 | AATACGAAATACTACAGCCCTGCTC | Antisense | 1144 |
| 204434_at | 299 | 963 | 3824 | TGCTCCCAGGTGTGCATCTTACTGG | Antisense | 1145 |
| 204724_s_at | 432 | 321 | 1896 | CACCAGCAAGGACGGCCAGGACGGT | Antisense | 1146 |
| 204724_s_at | 538 | 145 | 2007 | AGCCTGCCAAGGAGCCGTGTTAGGA | Antisense | 1147 |
| 204724_s_at | 1011 | 595 | 2043 | GAAATCAGGCTCTCGAAGCTCATAA | Antisense | 1148 |
| 204724_s_at | 984 | 569 | 2081 | GAAGCAAGTGACAAGGACGCCCGAA | Antisense | 1149 |
| 204724_s_at | 4 | 619 | 2096 | GACGCCCGAAGCACAGTGGACGGTC | Antisense | 1150 |
| 204724_s_at | 198 | 855 | 2113 | GGACGGTCATGAAGGAGCGGGGGTG | Antisense | 1151 |
| 204724_s_at | 188 | 761 | 2153 | GTCCAGGAGAGGGAGCGCCCCTGGC | Antisense | 1152 |
| 204724_s_at | 537 | 883 | 2232 | GGCTGTCGCCTGACAGCATACCTCA | Antisense | 1153 |
| 204724_s_at | 1032 | 519 | 2247 | GCATACCTCAAAAGGCCCTAGCTAA | Antisense | 1154 |
| 204724_s_at | 790 | 833 | 2323 | GGTTGTGTACAACTCCACGAGGTGA | Antisense | 1155 |
| 204724_s_at | 617 | 1045 | 2383 | TAAAGACTATGATCTCATCCCAATA | Antisense | 1156 |
| 204778_x_at | 415 | 437 | 338 | CCGGCTATGGGCTCGAGCCGAGTTC | Antisense | 1157 |
| 204778_x_at | 152 | 793 | 435 | GGGCGCCAAGGAGCAGAGGGACTCG | Antisense | 1158 |
| 204778_x_at | 177 | 857 | 453 | GGACTCGGACTTGGCGGCCGAGAGT | Antisense | 1159 |
| 204778_x_at | 550 | 889 | 468 | GGCCGAGAGTAACTTCCGGATCTAC | Antisense | 1160 |
| 204778_x_at | 155 | 1009 | 488 | TCTACCCCTGGATGCGAAGCTCAGG | Antisense | 1161 |
| 204778_x_at | 201 | 341 | 509 | CAGGAACTGACCGCAAACGAGGCCG | Antisense | 1162 |
| 204778_x_at | 569 | 417 | 547 | CGCTACCAGACCCTGGAGCTGGAGA | Antisense | 1163 |
| 204778_x_at | 1042 | 127 | 573 | AGAATTTCACTACAATCGCTACCTG | Antisense | 1164 |
| 204778_x_at | 714 | 161 | 628 | ACGCTCTGCCTCACGGAAAGACAGA | Antisense | 1165 |
| 204778_x_at | 1099 | 147 | 718 | ACCACCGGCCAAGACAGGGCTGAAG | Antisense | 1166 |
| 204778_x_at | 196 | 489 | 817 | GCCCAGCTCTGGGAACTGAATCAGG | Antisense | 1167 |
| 204897_at | 294 | 133 | 3030 | AGCAGCTTATTGTTTCTCTGAAAGT | Antisense | 1168 |
| 204897_at | 401 | 71 | 3112 | ATGGCAAGTTGCATCAGAAAGCTTT | Antisense | 1169 |
| 204897_at | 1120 | 685 | 3154 | GATTCCCAAACGTGGTTACATTAGC | Antisense | 1170 |
| 204897_at | 736 | 239 | 3162 | AACGTGGTTACATTAGCCATTCATG | Antisense | 1171 |
| 204897_at | 267 | 475 | 3177 | GCCATTCATGTATGTCAGAAGTGCA | Antisense | 1172 |
| 204897_at | 1002 | 909 | 3206 | TGGGGCACTTAATGGTCACCTTGTA | Antisense | 1173 |
| 204897_at | 560 | 735 | 3228 | GTAACAGTTTTGTGTAACTCCCAGT | Antisense | 1174 |
| 204897_at | 102 | 739 | 3241 | GTAACTCCCAGTGATGCTGTACACA | Antisense | 1175 |
| 204897_at | 629 | 499 | 3256 | GCTGTACACATATTTGAAGGGTCTT | Antisense | 1176 |
| 204897_at | 692 | 777 | 3315 | GTGTTTTTGTGAATTGCTTGGTTGT | Antisense | 1177 |
| 204897_at | 1018 | 197 | 3344 | AAATTCTGAGCCTGATATTGATATG | Antisense | 1178 |
| 204921_at | 539 | 489 | 2633 | GCCCACTGCCTAAATGATGCCCGGC | Antisense | 1179 |
| 204921_at | 691 | 529 | 2674 | GCAGATCCCAGTGAGTCATGAGCCT | Antisense | 1180 |
| 204921_at | 618 | 65 | 2735 | ATGTGGGTAGAAGCTTTCCTGCCCC | Antisense | 1181 |
| 204921_at | 59 | 379 | 2766 | CTCTAGTAGCCCTCAGTGTCGAAGG | Antisense | 1182 |
| 204921_at | 977 | 561 | 2786 | GAAGGTGAGCTTGTAGGTGCCTGCC | Antisense | 1183 |
| 204921_at | 884 | 621 | 2831 | GACCATCTGGGTCTGTGTAGCTGGG | Antisense | 1184 |
| 204921_at | 562 | 113 | 2872 | AGAGATGGGGACCAGAAGCCCCCCA | Antisense | 1185 |
| 204921_at | 522 | 443 | 2942 | CCTGCCACCTGTCAGACTTGGGTGA | Antisense | 1186 |
| 204921_at | 137 | 93 | 2975 | AGTGAGGCTGTTAGGTCCTGCAGTT | Antisense | 1187 |
| 204921_at | 664 | 701 | 2997 | GTTCCAGAGCAGTCTAGGGACACCA | Antisense | 1188 |
| 204921_at | 990 | 149 | 3018 | ACCACTGCCCTGTCTTTAGGAAATC | Antisense | 1189 |
| 204947_at | 859 | 401 | 1935 | CTGGCTGGGCGTGTAGGACGGTGAG | Antisense | 1190 |
| 204947_at | 879 | 1073 | 1948 | TAGGACGGTGAGAGCACTTCTGTCT | Antisense | 1191 |
| 204947_at | 1009 | 13 | 2010 | ATTTATTTATCGAGGCCTCTTTGGT | Antisense | 1192 |
| 204947_at | 268 | 387 | 2087 | CTCCCTCTACCCTTGAGCAAGGGCA | Antisense | 1193 |
| 204947_at | 807 | 863 | 2147 | GGAACTGAGGCCTGGGTGATTTATT | Antisense | 1194 |
| 204947_at | 1066 | 613 | 2198 | GACTGACTGACAGCCATGGGTGGTC | Antisense | 1195 |
| 204947_at | 404 | 815 | 2216 | GGTGGTCAGATGGTGGGGTGGGCCC | Antisense | 1196 |
| 204947_at | 990 | 503 | 2267 | GCTGCCCCCCAGGATGGATATGAGA | Antisense | 1197 |
| 204947_at | 523 | 907 | 2305 | TGGGGGACCTTCACTGATGTGGGCA | Antisense | 1198 |
| 204947_at | 832 | 159 | 2417 | ACCCTCCAATCTGCACTTTGATTTG | Antisense | 1199 |
| 204947_at | 249 | 935 | 2435 | TGATTTGCTTCCTAACAGCTCTGTT | Antisense | 1200 |
| 205000_at | 537 | 1039 | 3978 | TAAGCAGACCCGGCATTGGCAATGT | Antisense | 1201 |
| 205000_at | 127 | 879 | 3989 | GGCATTGGCAATGTAGCTGTAATTT | Antisense | 1202 |
| 205000_at | 1027 | 1099 | 4138 | TTTCTCTTGAGTTTTGTTATGTGTT | Antisense | 1203 |
| 205000_at | 813 | 693 | 4184 | GATTGTCTGGTATATTTACAGTCCT | Antisense | 1204 |
| 205000_at | 277 | 1103 | 4198 | TTTACAGTCCTCAAACATGGTTATT | Antisense | 1205 |
| 205000_at | 804 | 231 | 4211 | AACATGGTTATTTCTGTCAGTGACT | Antisense | 1206 |
| 205000_at | 437 | 233 | 4237 | AACATTCGGTTTTACCAGCCAGCAG | Antisense | 1207 |
| 205000_at | 507 | 151 | 4250 | ACCAGCCAGCAGTATTCTTCAGTAA | Antisense | 1208 |
| 205000_at | 306 | 281 | 4299 | AATCATTGAACCTCGAGTCACTGTA | Antisense | 1209 |
| 205000_at | 45 | 1087 | 4346 | TATTAGTTTTAGATGCTGGCACCGC | Antisense | 1210 |
| 205000_at | 106 | 873 | 4363 | GGCACCGCATGTGCTCTGTTTATTC | Antisense | 1211 |
| 205001_s_at | 796 | 127 | 1772 | AGAAGTGCCTTCTTGGTTGGAAAAT | Antisense | 1212 |
| 205001_s_at | 323 | 297 | 1817 | CAAGGGTGGCAGTCGTGGACGATCT | Antisense | 1213 |
| 205001_s_at | 321 | 691 | 1852 | GATTCAGTGGAGGATTTGGTGCCAG | Antisense | 1214 |
| 205001_s_at | 909 | 1079 | 1881 | TATCGACAAAGTAGTGGTTCCAGCA | Antisense | 1215 |
| 205001_s_at | 173 | 881 | 1911 | GGCTTTGGTGCTAGTCGCGGAAGCA | Antisense | 1216 |
| 205001_s_at | 122 | 881 | 1989 | GGCTATGGAGGCTTCTACAATAGTG | Antisense | 1217 |
| 205001_s_at | 1122 | 813 | 2056 | GGGGCAACTGAATCTGCTTTGCAGC | Antisense | 1218 |
| 205001_s_at | 550 | 367 | 2072 | CTTTGCAGCAAAGTCACCCTTACAA | Antisense | 1219 |
| 205001_s_at | 1015 | 497 | 2173 | GCTGTGATTCTCCTGATAATTCAAG | Antisense | 1220 |
| 205001_s_at | 846 | 1 | 2188 | ATAATTCAAGGGAGCTCAAAGTCAC | Antisense | 1221 |
| 205001_s_at | 442 | 109 | 2265 | AGATGTAATTGCTCTAGTTTGGATT | Antisense | 1222 |
| 205027_s_at | 290 | 15 | 2203 | ATTATATAACTCTTTGAGCCTTTAT | Antisense | 1223 |
| 205027_s_at | 997 | 625 | 2218 | GAGCCTTTATTGGTAAATTCTGGTA | Antisense | 1224 |
| 205027_s_at | 81 | 287 | 2260 | AATTTGGGTGACTAGAACAACTTGA | Antisense | 1225 |
| 205027_s_at | 652 | 729 | 2290 | GTAGCAATAAGCTGGACTAGTGTCC | Antisense | 1226 |
| 205027_s_at | 158 | 779 | 2309 | GTGTCCTAAAAATGGCTAACTGATG | Antisense | 1227 |
| 205027_s_at | 956 | 769 | 2364 | GTGACAGTTTCTTTTGTGTTCCTAT | Antisense | 1228 |
| 205027_s_at | 91 | 703 | 2381 | GTTCCTATGGAAACATTTTATACTG | Antisense | 1229 |
| 205027_s_at | 38 | 917 | 2599 | TGTGTATGTTTTATATCAAATGCCT | Antisense | 1230 |
| 205027_s_at | 22 | 1031 | 2614 | TCAAATGCCTTCATGAATCTTTCAT | Antisense | 1231 |
| 205027_s_at | 667 | 215 | 2712 | AAACCCAATACTTTTGTCCAATGTG | Antisense | 1232 |
| 205027_s_at | 1156 | 175 | 2721 | ACTTTTGTCCAATGTGGTTGGTCAA | Antisense | 1233 |
| 205105_at | 701 | 177 | 3547 | ACTTCTTCTCTCATGAATCATCCAG | Antisense | 1234 |
| 205105_at | 579 | 197 | 3585 | AAATAAGTTCTCACCTACCCTTGAG | Antisense | 1235 |
| 205105_at | 317 | 629 | 3607 | GAGCTGCAAGGTGAATTCTCTCCAT | Antisense | 1236 |
| 205105_at | 581 | 389 | 3626 | CTCCATTACAGTCATCTTTGCCTTG | Antisense | 1237 |
| 205105_at | 516 | 1147 | 3643 | TTGCCTTGTGACATTCATCTGGTTA | Antisense | 1238 |
| 205105_at | 830 | 67 | 3698 | ATGGGCACTCCAATGAGGCAGCCTT | Antisense | 1239 |
| 205105_at | 388 | 263 | 3737 | AAGGGTTTGATTGTCGGTTCTCTAG | Antisense | 1240 |
| 205105_at | 1013 | 89 | 3771 | AGGGCTGTTTTGTTCTACTACTCAG | Antisense | 1241 |
| 205105_at | 1113 | 921 | 3834 | TGTCGAAAGTCTCACACCTTCATCA | Antisense | 1242 |
| 205105_at | 25 | 1029 | 3856 | TCACTATCCTTGATGCATTCACCTC | Antisense | 1243 |
| 205105_at | 260 | 355 | 3983 | CATTGGCTTTTATACCTTTCTTGGT | Antisense | 1244 |
| 205204_at | 363 | 109 | 167 | AGATCCGAGTGCACTCGCGAGGCAA | Antisense | 1245 |
| 205204_at | 229 | 463 | 200 | CCACCGGTCACTTCATGGGCAAGAA | Antisense | 1246 |
| 205204_at | 297 | 801 | 281 | GGGACCAGCGACTGCAGCTGAGTCA | Antisense | 1247 |
| 205204_at | 580 | 649 | 300 | GAGTCATGATCTGCTCGGAATCCTC | Antisense | 1248 |
| 205204_at | 324 | 963 | 311 | TGCTCGGAATCCTCCTGCTAAAGAA | Antisense | 1249 |
| 205204_at | 608 | 535 | 364 | GCACCCCAAATCCAGTACAGGAGGC | Antisense | 1250 |
| 205204_at | 882 | 1073 | 424 | TAGGGGCAGACACAACAGCGTGGCT | Antisense | 1251 |
| 205204_at | 829 | 141 | 440 | AGCGTGGCTTAGATTGTGCCCACCC | Antisense | 1252 |
| 205204_at | 311 | 763 | 455 | GTGCCCACCCAGGGAAGGTGCTGAA | Antisense | 1253 |
| 205204_at | 1159 | 951 | 476 | TGAATGGGACCCTGTTGATGGCCCC | Antisense | 1254 |
| 205204_at | 541 | 279 | 525 | AATCTCTGTTACTCCATTACTGTGA | Antisense | 1255 |
| 205419_at | 665 | 323 | 1055 | CAGCCCCTGAAGAAAATTCACGTGA | Antisense | 1256 |
| 205419_at | 139 | 671 | 1095 | GATGATGATACATTCCAAGTCTTCA | Antisense | 1257 |
| 205419_at | 477 | 599 | 1171 | GACAAACTTTGCAGGACTTCCCTTA | Antisense | 1258 |
| 205419_at | 288 | 525 | 1181 | GCAGGACTTCCCTTATAAAGCAAAA | Antisense | 1259 |
| 205419_at | 1029 | 705 | 1211 | GTTCAGCTTCCAATTAGTATTCTTT | Antisense | 1260 |
| 205419_at | 465 | 1085 | 1236 | TATATTTCTTTCATTGGGCGCTTTC | Antisense | 1261 |
| 205419_at | 228 | 365 | 1256 | CTTTCCCATCTCCAACTCGGAAGTA | Antisense | 1262 |
| 205419_at | 104 | 565 | 1275 | GAAGTAAGCCCAAGAGAACAACATA | Antisense | 1263 |
| 205419_at | 952 | 551 | 1361 | GAATACACCAAAAGGAGGCGCTCTT | Antisense | 1264 |
| 205419_at | 451 | 1 | 1526 | AAATCCAAATTTCTCTCAATGTTAG | Antisense | 1265 |
| 205419_at | 469 | 649 | 1550 | GATTTAATTCCCTCAATAACACCAA | Antisense | 1266 |
| 205421_at | 282 | 293 | 2625 | CAATTCCTATTCATTCTTAGCACTT | Antisense | 1267 |
| 205421_at | 664 | 1133 | 2638 | TTCTTAGCACTTTGACATGTCTTGG | Antisense | 1268 |
| 205421_at | 242 | 593 | 2690 | GAAAGATCAATTATATCCATGCTTA | Antisense | 1269 |
| 205421_at | 450 | 515 | 2710 | GCTTAACAGGATCAGCAGGAGCTTT | Antisense | 1270 |
| 205421_at | 226 | 1033 | 2755 | TAATAAGGGATTGATCTTTCTTTTT | Antisense | 1271 |
| 205421_at | 515 | 1011 | 2773 | TCTTTTTTTGTTATCGAGGCTTTTG | Antisense | 1272 |
| 205421_at | 1119 | 781 | 2811 | GTGTGTTCTGCTTTATATGTTATAT | Antisense | 1273 |
| 205421_at | 615 | 1033 | 2836 | TCAATATCTTTTCAGATGCAGTCTA | Antisense | 1274 |
| 205421_at | 461 | 673 | 2850 | GATGCAGTCTATATTTTATGCTGAG | Antisense | 1275 |
| 205421_at | 1010 | 285 | 2972 | AATATACACATGTCGCGTAATCGTA | Antisense | 1276 |
| 205421_at | 572 | 943 | 3000 | TGAGCATCTTAGTCTTTAAAACACA | Antisense | 1277 |
| 205447_s_at | 697 | 483 | 2234 | GCCGTCACCCGAAGTCAGAAACGTG | Antisense | 1278 |
| 205447_s_at | 699 | 1027 | 2351 | TCACTATCTACCTTCAGCTCAGAGA | Antisense | 1279 |
| 205447_s_at | 985 | 267 | 2394 | AAGGCACAGCTAGTGAACCTTCCCC | Antisense | 1280 |
| 205447_s_at | 980 | 429 | 2416 | CCCCAGTGGCACACCTGAAGTTGGC | Antisense | 1281 |
| 205447_s_at | 530 | 533 | 2439 | GCAGCACCAACACTGATGAGCGGCC | Antisense | 1282 |
| 205447_s_at | 195 | 627 | 2468 | GAGCGGTCTGATGACATGTGCTCCC | Antisense | 1283 |
| 205447_s_at | 61 | 337 | 2492 | CAGGGCTCAGAAATCCCACTGGACC | Antisense | 1284 |
| 205447_s_at | 467 | 453 | 2562 | CCATTCCACACCAGGAACTTCTCAG | Antisense | 1285 |
| 205447_s_at | 971 | 485 | 2595 | GCCCTCCCAATTCTGAGGACTCAGA | Antisense | 1286 |
| 205447_s_at | 109 | 171 | 2643 | ACTCCAACAGCGTTGATGCCTTGCG | Antisense | 1287 |
| 205447_s_at | 785 | 949 | 2690 | TGAAAGCCACTCGTATTCCTTGTAC | Antisense | 1288 |
| 205518_s_at | 831 | 1027 | 1672 | TCAAGAATTCACTGATCTGATGCAA | Antisense | 1289 |
| 205518_s_at | 760 | 1079 | 1707 | TATCATTACATCTTGAACCCAGGAA | Antisense | 1290 |
| 205518_s_at | 705 | 1 | 1769 | ATAGATAATTCCACGTTGCCTTTGT | Antisense | 1291 |
| 205518_s_at | 470 | 163 | 1781 | ACGTTGCCTTTGTGATTTGTATATA | Antisense | 1292 |
| 205518_s_at | 429 | 23 | 1846 | ATATTTTACTTGTTCCATCCACAGT | Antisense | 1293 |
| 205518_s_at | 1070 | 1145 | 1858 | TTCCATCCACAGTTCTCTACAGAAA | Antisense | 1294 |
| 205518_s_at | 583 | 271 | 2006 | AATGGTCTGTTTTCGTGATATTCGT | Antisense | 1295 |
| 205518_s_at | 962 | 707 | 2070 | GTTAACTAGCATTCTGTTTTACATG | Antisense | 1296 |
| 205518_s_at | 263 | 345 | 2091 | CATGTTGACATTTCTAACACACACA | Antisense | 1297 |
| 205518_s_at | 1128 | 307 | 2108 | CACACACACCACTGATTTGAACTTC | Antisense | 1298 |
| 205518_s_at | 818 | 21 | 2151 | ATATATGCTAGGTCTGATTCTGAAG | Antisense | 1299 |
| 205668_at | 791 | 863 | 6476 | GGAACTCTAAACCTTGTGATGACTA | Antisense | 1300 |
| 205668_at | 475 | 37 | 6550 | ATCACTTTAAGTTTTGACACCTAGA | Antisense | 1301 |
| 205668_at | 859 | 749 | 6580 | GTCTTAGTAATAGCATCCACTGGAA | Antisense | 1302 |
| 205668_at | 914 | 1021 | 6623 | TCAGCATTTAACTTACATTTGTACT | Antisense | 1303 |
| 205668_at | 496 | 1105 | 6739 | TTTATATAGTCTACAGTTGTCCTGA | Antisense | 1304 |
| 205668_at | 868 | 105 | 6753 | AGTTGTCCTGATTTCTTATTGAATT | Antisense | 1305 |
| 205668_at | 328 | 551 | 6773 | GAATTTGTTAGACTAGTTCTCTTGT | Antisense | 1306 |
| 205668_at | 85 | 705 | 6788 | GTTCTCTTGTCTTGTGATCTGTGTA | Antisense | 1307 |
| 205668_at | 695 | 1103 | 6817 | TTTAGTCACTAAGACTTTCCTCCAA | Antisense | 1308 |
| 205668_at | 422 | 1097 | 6832 | TTTCCTCCAAGAACTAAGCCAACTT | Antisense | 1309 |
| 205668_at | 261 | 165 | 6869 | ACGGCTGTATATAATGGTGATGTCA | Antisense | 1310 |
| 205841_at | 615 | 41 | 4627 | ATCTATTTTATTATGGTTTCCCTTG | Antisense | 1311 |
| 205841_at | 296 | 829 | 4641 | GGTTTCCCTTGTATCTATTTGTGGT | Antisense | 1312 |
| 205841_at | 274 | 611 | 4707 | GACTACTATGAACAGTTTTCTTTTA | Antisense | 1313 |
| 205841_at | 237 | 53 | 4749 | ATGCCAGGAATATTGTCATCCTTTG | Antisense | 1314 |
| 205841_at | 582 | 613 | 4781 | GACTGCCAATAACATTCTTCGATCT | Antisense | 1315 |
| 205841_at | 937 | 33 | 4789 | ATAACATTCTTCGATCTCTGGGATT | Antisense | 1316 |
| 205841_at | 105 | 679 | 4801 | GATCTCTGGGATTTATGCTCATGAA | Antisense | 1317 |
| 205841_at | 770 | 837 | 4875 | GGATAGCTCATTAAGAAGTGCAGCA | Antisense | 1318 |
| 205841_at | 1018 | 93 | 4891 | AGTGCAGCAGGTTAAGAATTTTTTC | Antisense | 1319 |
| 205841_at | 949 | 549 | 4906 | GAATTTTTTCCTAAAGACTGTATAT | Antisense | 1320 |
| 205841_at | 610 | 583 | 5050 | GAAAATTCATAACGTGTATCTTTAA | Antisense | 1321 |
| 205856_at | 565 | 595 | 2175 | GAAATACTCAGCTTAGGCATTTTTA | Antisense | 1322 |
| 205856_at | 833 | 959 | 2236 | TGCATAGAATTGTTACCAACCTCCA | Antisense | 1323 |
| 205856_at | 224 | 929 | 2246 | TGTTACCAACCTCCAAAGGGCTCTT | Antisense | 1324 |
| 205856_at | 508 | 305 | 2259 | CAAAGGGCTCTTTAAAATCATATTT | Antisense | 1325 |
| 205856_at | 420 | 1087 | 2287 | TATTCATTTGAGGATGTCTTATAAA | Antisense | 1326 |
| 205856_at | 1144 | 83 | 2324 | AGGTCAGAATGCTTACGGGTGTTAT | Antisense | 1327 |
| 205856_at | 990 | 325 | 2328 | CAGAATGCTTACGGGTGTTATTTTT | Antisense | 1328 |
| 205856_at | 55 | 1039 | 2354 | TAAGTTGTTGAATTCCTTAATTTAG | Antisense | 1329 |
| 205856_at | 1136 | 507 | 2383 | GCTCATTATTTTTTGCACACTCACA | Antisense | 1330 |
| 205856_at | 807 | 1135 | 2411 | TTCTCTCTCAGAAATCAATGGCATT | Antisense | 1331 |
| 205856_at | 963 | 1029 | 2425 | TCAATGGCATTTGAACCACCAAAAA | Antisense | 1332 |
| 205987_at | 519 | 461 | 720 | CCAGCTGTTGCTGGTTTGTCATGCC | Antisense | 1333 |
| 205987_at | 890 | 849 | 795 | GGAGCAACTGGGCACTAAACATGGT | Antisense | 1334 |
| 205987_at | 785 | 495 | 889 | GCTGGCCTGTCTTGTCGAGTGAGAC | Antisense | 1335 |
| 205987_at | 1067 | 475 | 979 | GCCTTGGTAGTGATAGTGCCCTTGG | Antisense | 1336 |
| 205987_at | 600 | 763 | 994 | GTGCCCTTGGTGATTCTAATAGTCC | Antisense | 1337 |
| 205987_at | 1044 | 569 | 1035 | GAAGCACTGCTCATATCAGGACATC | Antisense | 1338 |
| 205987_at | 992 | 1019 | 1050 | TCAGGACATCCTGTGAGACTCTTCC | Antisense | 1339 |
| 205987_at | 702 | 945 | 1078 | TGACTCCCCCATTGTGTTAAGAACC | Antisense | 1340 |
| 205987_at | 679 | 455 | 1102 | CCAGCAACCCAGGAGCCTAGTACAA | Antisense | 1341 |
| 205987_at | 674 | 285 | 1125 | AATATAGTGATGCCATCCCGTCGAC | Antisense | 1342 |
| 205987_at | 992 | 435 | 1142 | CCGTCGACTCTCCATTTAAATTGTT | Antisense | 1343 |
| 205997_at | 57 | 665 | 2202 | GATGGTAAAGGCTGTTCAACCCCAA | Antisense | 1344 |
| 205997_at | 488 | 499 | 2212 | GCTGTTCAACCCCAAGAGATGAGTC | Antisense | 1345 |
| 205997_at | 841 | 649 | 2232 | GAGTCAGATGAAGCCCCATGTGTAT | Antisense | 1346 |
| 205997_at | 110 | 425 | 2246 | CCCATGTGTATGATCTGCCAGTAGA | Antisense | 1347 |
| 205997_at | 628 | 473 | 2262 | GCCAGTAGAAGGCAATGAGCCCCCA | Antisense | 1348 |
| 205997_at | 849 | 349 | 2296 | CATAAAGACACAAACGCACTTCCCC | Antisense | 1349 |
| 205997_at | 894 | 837 | 2334 | GGATAATCCAATGTCTACACCTAAG | Antisense | 1350 |
| 205997_at | 719 | 751 | 2346 | GTCTACACCTAAGGACTCAAATCCA | Antisense | 1351 |
| 205997_at | 23 | 1055 | 2455 | TACTATCTCACCAGTATTTGCTCTC | Antisense | 1352 |
| 205997_at | 768 | 721 | 2468 | GTATTTGCTCTCGACTCAAGAAGGT | Antisense | 1353 |
| 205997_at | 257 | 581 | 2548 | GAACATATGCCTGAGAACCTTTGCA | Antisense | 1354 |
| 206118_at | 674 | 501 | 2008 | GCTGACATCCTGCGAGACTACAAAG | Antisense | 1355 |
| 206118_at | 1162 | 905 | 2052 | TCCTGAAAACCCTCTGAAGTACCTA | Antisense | 1356 |
| 206118_at | 413 | 565 | 2067 | GAAGTACCTATATCCTGACATTCCC | Antisense | 1357 |
| 206118_at | 568 | 303 | 2097 | CAAAGCCTTCGGTAAACACTACAGC | Antisense | 1358 |
| 206118_at | 459 | 507 | 2120 | GCTCTCAGCCTTGCGAAGTTTCAAG | Antisense | 1359 |
| 206118_at | 553 | 987 | 2189 | TCCCCATCTCAACAATCCGAAGTGA | Antisense | 1360 |
| 206118_at | 103 | 215 | 2283 | AAACCTGAGTCCCACAACAATTGAA | Antisense | 1361 |
| 206118_at | 116 | 955 | 2310 | TGCAATGAAGTCTCCTTATTCTGCT | Antisense | 1362 |
| 206118_at | 52 | 121 | 2390 | AGACTGTTCTTTGCCCAATAACCAC | Antisense | 1363 |
| 206118_at | 366 | 607 | 2461 | GACATCTGAAGCTCTCTTCACACTC | Antisense | 1364 |
| 206118_at | 787 | 989 | 2484 | TCCCGTGGCACTCCTCAATTGGGAG | Antisense | 1365 |
| 206618_at | 1048 | 569 | 2935 | GAAGCATATCCAGAGGGCGAAAGAT | Antisense | 1366 |
| 206618_at | 622 | 885 | 2950 | GGCGAAAGATATCTCTCCATTGTGC | Antisense | 1367 |
| 206618_at | 25 | 349 | 2974 | CATCTGCCTCTTTTGACGTTGGAAG | Antisense | 1368 |
| 206618_at | 812 | 865 | 2994 | GGAAGACACATGTCTTACTCCCCAA | Antisense | 1369 |
| 206618_at | 498 | 1057 | 3009 | TACTCCCCAAAGGGAGCCCAGCACT | Antisense | 1370 |
| 206618_at | 317 | 853 | 3035 | GGAGCCTTCTTGATGATCTCAAAAA | Antisense | 1371 |
| 206618_at | 3 | 771 | 3086 | GTGACTGTGAAACCGTCAGTTCGGA | Antisense | 1372 |
| 206618_at | 182 | 589 | 3094 | GAAACCGTCAGTTCGGAAGGCTGGT | Antisense | 1373 |
| 206618_at | 601 | 559 | 3109 | GAAGGCTGGTTAGAACATGTGGGAG | Antisense | 1374 |
| 206618_at | 804 | 765 | 3253 | GTGCTTACAATTTTGTGTACCAACT | Antisense | 1375 |
| 206618_at | 118 | 689 | 3364 | GATTTCATTGCTATTGCATAGTCTC | Antisense | 1376 |
| 206637_at | 64 | 945 | 1936 | TGAGCCTGGGGTTCTGGTGTTAGAA | Antisense | 1377 |
| 206637_at | 1091 | 1089 | 1995 | TATTGGCATACGTTATCAGCAACTT | Antisense | 1378 |
| 206637_at | 1051 | 173 | 2016 | ACTTCCCCTGTTCAATAGTATGGGA | Antisense | 1379 |
| 206637_at | 69 | 309 | 2065 | CACACCCACACCGTAGAACATATAT | Antisense | 1380 |
| 206637_at | 1031 | 307 | 2204 | CACACATCACATTTTTCTGGAAAAC | Antisense | 1381 |
| 206637_at | 1129 | 615 | 2230 | GACGGATTTTACTTCTGGAGACATG | Antisense | 1382 |
| 206637_at | 150 | 637 | 2247 | GAGACATGGCATACGGTTACTGACT | Antisense | 1383 |
| 206637_at | 148 | 707 | 2262 | GTTACTGACTTATGAGCTACCAAAA | Antisense | 1384 |
| 206637_at | 578 | 1099 | 2295 | TTTCTCTGCTATTAACTGGCTAGAA | Antisense | 1385 |
| 206637_at | 119 | 127 | 2316 | AGAAGACATTCATCTATTTTTCAAA | Antisense | 1386 |
| 206637_at | 525 | 719 | 2370 | GTTTGTATCTATTTCATGCTTTACT | Antisense | 1387 |
| 206700_s_at | 79 | 1013 | 4952 | TCTTAAATCCTACTATCTCCTGGCC | Antisense | 1388 |
| 206700_s_at | 750 | 387 | 4968 | CTCCTGGCCTGGACCTCAGAAGGAG | Antisense | 1389 |
| 206700_s_at | 317 | 851 | 4989 | GGAGCTTTTTGCCTATCTATAATTT | Antisense | 1390 |
| 206700_s_at | 918 | 973 | 5019 | TGCCAATTTTTGATATCCTCTCTCC | Antisense | 1391 |
| 206700_s_at | 581 | 309 | 5078 | CACACCCCGATGCTCAGAAGTGTCT | Antisense | 1392 |
| 206700_s_at | 543 | 563 | 5094 | GAAGTGTCTTGCCAGCAACATTCCT | Antisense | 1393 |
| 206700_s_at | 1115 | 843 | 5130 | GGAGTGATTTCCTAAACCAGTTTCA | Antisense | 1394 |
| 206700_s_at | 741 | 489 | 5191 | GCCCAAGTGCGTACCTTTATTTTTG | Antisense | 1395 |
| 206700_s_at | 626 | 425 | 5225 | CCCTCTTTCTGTACTTCAATTTTTG | Antisense | 1396 |
| 206700_s_at | 794 | 819 | 5289 | GGTGCTTCAAAACTTGTACCTTCAT | Antisense | 1397 |
| 206700_s_at | 1014 | 57 | 5409 | ATGACTCTACCTGTGGTCTATCTTT | Antisense | 1398 |
| 207063_at | 609 | 931 | 937 | TGTTGGGAATTGGTACTGGCTAGAA | Antisense | 1399 |
| 207063_at | 768 | 1093 | 1025 | TTTGACAGCCACAGCATGTTCCGAA | Antisense | 1400 |
| 207063_at | 717 | 697 | 1121 | GTTGTACAACATGCCCTACAGCTAC | Antisense | 1401 |
| 207063_at | 899 | 373 | 1136 | CTACAGCTACTCTTTAAATCTCCAA | Antisense | 1402 |
| 207063_at | 182 | 559 | 1222 | GAATGGGATGAGCACTCCTACAAAA | Antisense | 1403 |
| 207063_at | 1040 | 265 | 1246 | AAGGATTCCAGAGAGCTCGCTTGCT | Antisense | 1404 |
| 207063_at | 564 | 593 | 1318 | GAAAGTGGGTCCCCACCAGACATTG | Antisense | 1405 |
| 207063_at | 284 | 121 | 1335 | AGACATTGAATCTGCCGCATCTTGA | Antisense | 1406 |
| 207063_at | 712 | 391 | 1346 | CTGCCGCATCTTGATACTGGACTTC | Antisense | 1407 |
| 207063_at | 780 | 1113 | 1407 | TTATTACCTGTCCAGTATCTTTGGT | Antisense | 1408 |
| 207063_at | 470 | 585 | 1461 | GAAAACACCAGAGGCCATACCTAAT | Antisense | 1409 |
| 207651_at | 757 | 1147 | 828 | TTGCCTTGTAATTCGACAGCTCTAC | Antisense | 1410 |
| 207651_at | 1133 | 175 | 906 | ACTTTTAGTGACCACGGGCTACATC | Antisense | 1411 |
| 207651_at | 103 | 795 | 921 | GGGCTACATCATATGCTTTGTTCCT | Antisense | 1412 |
| 207651_at | 23 | 31 | 968 | ATACCCTCAGCCAGACAGAAGTCAT | Antisense | 1413 |
| 207651_at | 997 | 563 | 985 | GAAGTCATAACTGATTGCTCAACCA | Antisense | 1414 |
| 207651_at | 1084 | 961 | 1000 | TGCTCAACCAGGATTTCACTCTTCA | Antisense | 1415 |
| 207651_at | 572 | 245 | 1025 | AAGCCAAAGAGGCTACACTGCTCCT | Antisense | 1416 |
| 207651_at | 1026 | 961 | 1066 | TGCTTTGATCCTATCCTGTACTATC | Antisense | 1417 |
| 207651_at | 257 | 211 | 1099 | AAAGCATTCCGCTCAAAGGTCACTG | Antisense | 1418 |
| 207651_at | 1005 | 1027 | 1118 | TCACTGAGACTTTTGCCTCACCTAA | Antisense | 1419 |
| 207651_at | 800 | 959 | 1208 | TGCTACCAATTCTGGCCTTACTGGA | Antisense | 1420 |
| 207714_s_at | 400 | 601 | 1348 | GACACATGGGTGCTATTGGGGTTGG | Antisense | 1421 |
| 207714_s_at | 450 | 909 | 1370 | TGGGGGGGAGGTGAGGTACCAGCCT | Antisense | 1422 |
| 207714_s_at | 1020 | 83 | 1383 | AGGTACCAGCCTTGGATACTCCATG | Antisense | 1423 |
| 207714_s_at | 620 | 899 | 1424 | TGGACATGGGCCCCAGATACCATGA | Antisense | 1424 |
| 207714_s_at | 520 | 683 | 1439 | GATACCATGATGCTGAGCCCGGAAA | Antisense | 1425 |
| 207714_s_at | 152 | 77 | 1458 | CGGAAACTCCACATCCTGTGGGACC | Antisense | 1426 |
| 207714_s_at | 464 | 917 | 1474 | TGTGGGACCTGGGCCATAGTCATTC | Antisense | 1427 |
| 207714_s_at | 99 | 397 | 1507 | CTGAAAGTCCCAGATCAAGCCTGCC | Antisense | 1428 |
| 207714_s_at | 957 | 299 | 1522 | CAAGCCTGCCTCAATCAGTATTCAT | Antisense | 1429 |
| 207714_s_at | 682 | 17 | 1547 | ATTTATAGCCAGGTACCTTCTCACC | Antisense | 1430 |
| 207714_s_at | 528 | 153 | 1561 | ACCTTCTCACCTGTGAGACCAAATT | Antisense | 1431 |
| 208296_x_at | 1080 | 1151 | 1417 | TTGAGTTCTCCTTTTAAGTACCAAT | Antisense | 1432 |
| 208296_x_at | 428 | 811 | 1469 | GGTGTGTCATTGCCTTGAAATGCTT | Antisense | 1433 |
| 208296_x_at | 537 | 595 | 1485 | GAAATGCTTGCTTAGGGCTTCTTTT | Antisense | 1434 |
| 208296_x_at | 564 | 1073 | 1497 | TAGGGCTTCTTTTATGTTATCTTAA | Antisense | 1435 |
| 208296_x_at | 1109 | 261 | 1523 | AAGTGCTGGTGAATTTTCCATTTTT | Antisense | 1436 |
| 208296_x_at | 683 | 1145 | 1538 | TTCCATTTTTTACATCCATTTCACA | Antisense | 1437 |
| 208296_x_at | 857 | 593 | 1627 | GAAAGGTAACAATCTTCATTCTACA | Antisense | 1438 |
| 208296_x_at | 713 | 275 | 1727 | AATGCGTGCGTTTCAGTGTTTAAGA | Antisense | 1439 |
| 208296_x_at | 1046 | 297 | 1843 | CAAGCAGTTTGTGTGGTGTTTCTAT | Antisense | 1440 |
| 208296_x_at | 1051 | 201 | 1928 | AAATCTGCTGGCCAGCTATGTCCTC | Antisense | 1441 |
| 208296_x_at | 1016 | 1071 | 1953 | TAGGAAATGACAGACCCAACCACCA | Antisense | 1442 |
| 208498_s_at | 84 | 1041 | 948 | TAACCATGACAATCAACGAGGACAT | Antisense | 1443 |
| 208498_s_at | 882 | 707 | 1165 | GTTACTATTAATCCAGACACTACTT | Antisense | 1444 |
| 208498_s_at | 658 | 911 | 1201 | TGGGTCTGTGAACATCGATGGCGCC | Antisense | 1445 |
| 208498_s_at | 930 | 993 | 1215 | TCGATGGCGCCAAATAAGGAACATG | Antisense | 1446 |
| 208498_s_at | 1058 | 977 | 1247 | TCCGCAATGTAGTGGATGGCCAGCC | Antisense | 1447 |
| 208498_s_at | 497 | 727 | 1255 | GTAGTGGATGGCCAGCCTTTTACAA | Antisense | 1448 |
| 208498_s_at | 520 | 933 | 1287 | TGATAATGGGAGCAACCAAGTGGCT | Antisense | 1449 |
| 208498_s_at | 922 | 773 | 1406 | GTGATGTCATTTCTGGAGATAAAAT | Antisense | 1450 |
| 208498_s_at | 802 | 1059 | 1459 | TACGTTTCTGATGATGGCAAAGCTC | Antisense | 1451 |
| 208498_s_at | 761 | 99 | 1495 | AGTAACTCTGCTGAAGATCCATTTA | Antisense | 1452 |
| 208498_s_at | 708 | 1113 | 1517 | TTATTGCAATTCATGCTGAATCTAA | Antisense | 1453 |
| 208688_x_at | 822 | 943 | 2576 | TGAGCTACAGGACTCCCGAGTGTGA | Antisense | 1454 |
| 208688_x_at | 703 | 905 | 2720 | TGGATTCTGCCATTGCGACACATTT | Antisense | 1455 |
| 208688_x_at | 754 | 491 | 2734 | GCGACACATTTTTGTGCCTTTCAGC | Antisense | 1456 |
| 208688_x_at | 392 | 145 | 2756 | AGCCCCTGGTGTCTGCAGTGGGGGA | Antisense | 1457 |
| 208688_x_at | 613 | 335 | 2771 | CAGTGGGGGATTTAAGGCACCCGCT | Antisense | 1458 |
| 208688_x_at | 866 | 517 | 2793 | GCTTCCACTTCTTTCTTGTTTGGAG | Antisense | 1459 |
| 208688_x_at | 1145 | 1115 | 2812 | TTGGAGTTTTCTGTTGGAACCGCCG | Antisense | 1460 |
| 208688_x_at | 173 | 883 | 2842 | GGCTCCGAAGACTTAGCGACGCCAC | Antisense | 1461 |
| 208688_x_at | 341 | 401 | 2909 | CTGTACACAGCCGAGCAGCATTTCC | Antisense | 1462 |
| 208688_x_at | 932 | 985 | 2931 | TCCGTTGAAGGACTTGCATCCCCAT | Antisense | 1463 |
| 208688_x_at | 391 | 311 | 3017 | CACCGTGCAGGTTGTGGCCGGTTTT | Antisense | 1464 |
| 208799_at | 1121 | 407 | 511 | CTGGCCTCTACTACGTGGACAGTGA | Antisense | 1465 |
| 208799_at | 55 | 1075 | 568 | TAGGTTCTGGCTCTGTGTATGCATA | Antisense | 1466 |
| 208799_at | 509 | 903 | 601 | TGGATCGGGGCTATTCCTATGACCT | Antisense | 1467 |
| 208799_at | 757 | 849 | 632 | GGAGCAGGCCTATGATCTGGCCCGT | Antisense | 1468 |
| 208799_at | 857 | 81 | 695 | AGGAGGTGCAGTCAACCTCTACCAC | Antisense | 1469 |
| 208799_at | 941 | 843 | 725 | GGAGGATGGCTGGATCCGAGTCTCC | Antisense | 1470 |
| 208799_at | 569 | 941 | 773 | TGAGAAGTATAGTGGCTCTACCCCC | Antisense | 1471 |
| 208799_at | 649 | 777 | 823 | GTGTTTCTTGGGGTGACTGTCATTG | Antisense | 1472 |
| 208799_at | 694 | 1033 | 849 | TAATACGGACACAGTGACCCATCCT | Antisense | 1473 |
| 208799_at | 69 | 563 | 891 | GAAGGGCCTTCAATTGTATCAGTAC | Antisense | 1474 |
| 208799_at | 511 | 1041 | 923 | TAAGCTCTGGCACATTGACCTCTAT | Antisense | 1475 |
| 208894_at | 950 | 413 | 356 | CGATCACCAATGTACCTCCAGAGGT | Antisense | 1476 |
| 208894_at | 87 | 681 | 357 | GATCACCAATGTACCTCCAGAGGTA | Antisense | 1477 |
| 208894_at | 907 | 443 | 370 | CCTCCAGAGGTAACTGTGCTCACGA | Antisense | 1478 |
| 208894_at | 549 | 975 | 372 | TCCAGAGGTAACTGTGCTCACGAAC | Antisense | 1479 |
| 208894_at | 674 | 83 | 377 | AGGTAACTGTGCTCACGAACAGCCC | Antisense | 1480 |
| 208894_at | 316 | 963 | 386 | TGCTCACGAACAGCCCTGTGGAACT | Antisense | 1481 |
| 208894_at | 98 | 899 | 404 | TGGAACTGAGAGAGCCCAACGTCCT | Antisense | 1482 |
| 208894_at | 283 | 301 | 420 | CAACGTCCTCATCTGTTTCATAGAC | Antisense | 1483 |
| 208894_at | 812 | 163 | 422 | ACGTCCTCATCTGTTTCATAGACAA | Antisense | 1484 |
| 208894_at | 687 | 759 | 424 | GTCCTCATCTGTTTCATAGACAAGT | Antisense | 1485 |
| 208894_at | 1109 | 399 | 432 | CTGTTTCATAGACAAGTTCACCCCA | Antisense | 1486 |
| 209078_s_at | 545 | 141 | 20 | AGCGACTTCTTCTGAGGAGGTTCCT | Antisense | 1487 |
| 209078_s_at | 383 | 421 | 114 | CCCACAATGCAGTCCTGGTGGCCTG | Antisense | 1488 |
| 209078_s_at | 31 | 909 | 129 | TGGTGGCCTGACTGTAACACCCAAC | Antisense | 1489 |
| 209078_s_at | 821 | 461 | 154 | CCAGCCCGGACAATATACACCACGA | Antisense | 1490 |
| 209078_s_at | 207 | 659 | 177 | GAGGATCTCCTTGACAACCTTTAAT | Antisense | 1491 |
| 209078_s_at | 1110 | 663 | 208 | GATGGACCTGACTTTCAAGACCGAG | Antisense | 1492 |
| 209078_s_at | 15 | 787 | 253 | GTGGTTGTGGATTTCCACGCACAGT | Antisense | 1493 |
| 209078_s_at | 893 | 185 | 273 | ACAGTGGTGTGGACCCTGCAAGATC | Antisense | 1494 |
| 209078_s_at | 30 | 309 | 373 | CACACAGACCTCGCCATTGAGTATG | Antisense | 1495 |
| 209078_s_at | 505 | 941 | 390 | TGAGTATGAGGTGTCAGCGGTGCCC | Antisense | 1496 |
| 209078_s_at | 62 | 969 | 410 | TGCCCACTGTGCTGGCCATGAAGAA | Antisense | 1497 |
| 209129_at | 406 | 893 | 1170 | GGCCACCCTGGAGAAATGTGCCACG | Antisense | 1498 |
| 209129_at | 200 | 903 | 1178 | TGGAGAAATGTGCCACGTGCTCCCA | Antisense | 1499 |
| 209129_at | 766 | 493 | 1224 | GCGGGCTATGGGGAAGGCCTACCAC | Antisense | 1500 |
| 209129_at | 805 | 1127 | 1300 | TTCACAGTGGATGCTACGAGCCAGA | Antisense | 1501 |
| 209129_at | 643 | 511 | 1312 | GCTACGAGCCAGATCCACTGTATTG | Antisense | 1502 |
| 209129_at | 921 | 675 | 1323 | GATCCACTGTATTGAGGACTTTCAC | Antisense | 1503 |
| 209129_at | 65 | 363 | 1341 | CTTTCACAGGAAGTTTGCCCCAAGA | Antisense | 1504 |
| 209129_at | 194 | 435 | 1358 | CCCCAAGATGCTCAGTGTGCGGTGG | Antisense | 1505 |
| 209129_at | 786 | 625 | 1396 | GAGCCAGGTCAGGAGGAGACTGTGA | Antisense | 1506 |
| 209129_at | 16 | 773 | 1417 | GTGAGAATTGTTGCTCTGGATCGAA | Antisense | 1507 |
| 209129_at | 313 | 903 | 1433 | TGGATCGAAGTTTTCACATTGGCTG | Antisense | 1508 |
| 209150_s_at | 288 | 331 | 1594 | CAGTTGTCTGGGGAGGATTACCGCT | Antisense | 1509 |
| 209150_s_at | 246 | 885 | 1625 | GGCGATCTGTGCTGAGTGTTGGCTC | Antisense | 1510 |
| 209150_s_at | 816 | 1013 | 1664 | TCTTCCTCTACTCAGTTTTCTATTA | Antisense | 1511 |
| 209150_s_at | 450 | 1055 | 1672 | TACTCAGTTTTCTATTATGCCCGGC | Antisense | 1512 |
| 209150_s_at | 802 | 417 | 1696 | CGCTCCAACATGTCTGGGGCAGTAC | Antisense | 1513 |
| 209150_s_at | 1025 | 333 | 1724 | CAGTAGAGTTCTTCGGCTACTCCTT | Antisense | 1514 |
| 209150_s_at | 570 | 1057 | 1741 | TACTCCTTACTCACTGGTTATGTCT | Antisense | 1515 |
| 209150_s_at | 439 | 1009 | 1798 | TCTTCCCTAAAGTTCATCCGGTATA | Antisense | 1516 |
| 209150_s_at | 1134 | 665 | 1839 | GATGGACTGAGTTCTGTATGGCAGA | Antisense | 1517 |
| 209150_s_at | 357 | 873 | 1858 | GGCAGAACTATTGCTGTTCTCTCCC | Antisense | 1518 |
| 209150_s_at | 1097 | 103 | 2047 | AGTTGCCTTTAGTTTTGGTCCTGAT | Antisense | 1519 |
| 209254_at | 70 | 809 | 1066 | GGGTCATTGTTTAAGATCTGGCTGG | Antisense | 1520 |
| 209254_at | 82 | 47 | 1081 | ATCTGGCTGGTGGTACCTAGCCTGC | Antisense | 1521 |
| 209254_at | 999 | 731 | 1093 | GTACCTAGCCTGCTGGAACTGGCAT | Antisense | 1522 |
| 209254_at | 702 | 955 | 1175 | TGCACCTTGGACTCACACAGGGACT | Antisense | 1523 |
| 209254_at | 360 | 307 | 1190 | CACAGGGACTCATCGAACGCTTGAA | Antisense | 1524 |
| 209254_at | 1148 | 637 | 1262 | GAGACTCCTTTATTTATGGGCAGTG | Antisense | 1525 |
| 209254_at | 1085 | 651 | 1306 | GATTGGTTACCCTGATCAAGGCCTT | Antisense | 1526 |
| 209254_at | 295 | 715 | 1375 | GTTTATGGACATCTCACTTTCCCAC | Antisense | 1527 |
| 209254_at | 913 | 983 | 1425 | TCCTGACCCATTACATGCACATGTA | Antisense | 1528 |
| 209254_at | 736 | 345 | 1438 | CATGCACATGTACTCACATACTCCC | Antisense | 1529 |
| 209254_at | 236 | 427 | 1460 | CCCTCTTCCTTCTCGATGGAGTTAA | Antisense | 1530 |
| 209262_s_at | 698 | 995 | 1347 | TCGGGCCAGTGACCATGACGGGGCC | Antisense | 1531 |
| 209262_s_at | 596 | 799 | 1408 | GGGACAGGGAATGCTGAGGCCTCGA | Antisense | 1532 |
| 209262_s_at | 248 | 255 | 1539 | AAGAGACTGATCATCCAGGCCTCAG | Antisense | 1533 |
| 209262_s_at | 1018 | 401 | 1584 | CTGTCCAGGATGGAGGGTCCAATCC | Antisense | 1534 |
| 209262_s_at | 647 | 809 | 1598 | GGGTCCAATCCTAGGACAGCCTTGT | Antisense | 1535 |
| 209262_s_at | 806 | 1023 | 1626 | TCAGCACCCCTAGCATGAACTTGTG | Antisense | 1536 |
| 209262_s_at | 61 | 665 | 1678 | GATGGACAAAGGCCTGGCGTCGGCC | Antisense | 1537 |
| 209262_s_at | 884 | 335 | 1716 | CAGTGGGCAGGGGTAGCTAGCGTGT | Antisense | 1538 |
| 209262_s_at | 51 | 511 | 1731 | GCTAGCGTGTGCCAGGCAGATCCTC | Antisense | 1539 |
| 209262_s_at | 658 | 331 | 1747 | CAGATCCTCTGGACACGTAACCTAT | Antisense | 1540 |
| 209262_s_at | 472 | 189 | 1759 | ACACGTAACCTATGTCAGACACTAC | Antisense | 1541 |
| 209277_at | 168 | 773 | 1154 | GTGAGACTGAATTCTTGCAATGCAT | Antisense | 1542 |
| 209277_at | 693 | 57 | 1196 | ATGACTCACTCATTTCTTGGGGTCG | Antisense | 1543 |
| 209277_at | 40 | 1029 | 1201 | TCACTCATTTCTTGGGGTCGTATTC | Antisense | 1544 |
| 209277_at | 826 | 1159 | 1212 | TTGGGGTCGTATTCCTGATTTCAGA | Antisense | 1545 |
| 209277_at | 199 | 253 | 1261 | AAGACAATATAATCATGTGCTTTTA | Antisense | 1546 |
| 209277_at | 246 | 15 | 1345 | ATTATATTTTAGTCCCAAGAAGACA | Antisense | 1547 |
| 209277_at | 635 | 565 | 1363 | GAAGACAAAGTCGCAGATTAACAAC | Antisense | 1548 |
| 209277_at | 631 | 97 | 1371 | AGTCGCAGATTAACAACACTTTAAA | Antisense | 1549 |
| 209277_at | 50 | 171 | 1402 | ACTCCCATTTTATTGTGTTAGATTG | Antisense | 1550 |
| 209277_at | 4 | 679 | 1462 | GATCTTAACTTTCTTGACTGATTTA | Antisense | 1551 |
| 209277_at | 309 | 981 | 1522 | TCCTCTGGGAAAGCAATTTCTATCT | Antisense | 1552 |
| 209606_at | 595 | 493 | 1164 | GAATTGCAAAACTGACATCCCATTT | Antisense | 1553 |
| 209606_at | 140 | 169 | 1174 | ACTGACATCCCATTTCACAGCAATA | Antisense | 1554 |
| 209606_at | 1008 | 309 | 1189 | CACAGCAATAGTGACCTTTATTTAA | Antisense | 1555 |
| 209606_at | 778 | 697 | 1217 | GTTGTGTTATAGTTTATGCTTCTTA | Antisense | 1556 |
| 209606_at | 422 | 1099 | 1249 | TTTCAACCTAAACAGCCAATTTCTA | Antisense | 1557 |
| 209606_at | 735 | 189 | 1260 | ACAGCCAATTTCTAAGCAGACAGGA | Antisense | 1558 |
| 209606_at | 240 | 1087 | 1369 | TATTTTCTAGATTATCCCTGTGAAT | Antisense | 1559 |
| 209606_at | 292 | 201 | 1485 | AAATCTTAGGTTTGCTTATGCCCAG | Antisense | 1560 |
| 209606_at | 775 | 499 | 1590 | GCTGTTTCTCACATCTATAGTGACA | Antisense | 1561 |
| 209606_at | 195 | 25 | 1639 | ATAGGAGGGGTTAAGGTTCATGAGA | Antisense | 1562 |
| 209606_at | 790 | 823 | 1679 | GGTCTGAGATGGGTGCTGCAAAGAT | Antisense | 1563 |
| 209728_at | 847 | 523 | 690 | GCATCCAAGCATGATGAGCCCTCTC | Antisense | 1564 |
| 209728_at | 29 | 347 | 691 | CATCCAAGCATGATGAGCCCTCTCA | Antisense | 1565 |
| 209728_at | 319 | 145 | 706 | AGCCCTCTCACGGTGCAATGGAGTG | Antisense | 1566 |
| 209728_at | 77 | 487 | 707 | GCCCTCTCACGGTGCAATGGAGTGC | Antisense | 1567 |
| 209728_at | 329 | 427 | 708 | CCCTCTCACGGTGCAATGGAGTGCA | Antisense | 1568 |
| 209728_at | 189 | 547 | 720 | GCAATGGAGTGCACGGTCTGAATCT | Antisense | 1569 |
| 209728_at | 343 | 149 | 854 | AGCCAACAGGACTCTTGAGCTGAAG | Antisense | 1570 |
| 209728_at | 233 | 433 | 1143 | CCCCTTGTGCCACACATTGCATTAT | Antisense | 1571 |
| 209728_at | 341 | 425 | 1144 | CCCTTGTGCCACACATTGCATTATT | Antisense | 1572 |
| 209728_at | 512 | 357 | 1146 | CTTGTGCCACACATTGCATTATTAA | Antisense | 1573 |
| 209728_at | 600 | 761 | 1149 | GTGCCACACATTGCATTATTAAATG | Antisense | 1574 |
| 209795_at | 451 | 1071 | 1143 | TAGTCTAATTGAATCCCTTAAACTC | Antisense | 1575 |
| 209795_at | 691 | 67 | 1273 | ATGGGATGATCGTGTATTTATTTTT | Antisense | 1576 |
| 209795_at | 665 | 1107 | 1294 | TTTTTTACTTCCTCAGCTGTAGACA | Antisense | 1577 |
| 209795_at | 1000 | 177 | 1300 | ACTTCCTCAGCTGTAGACAGGTCCT | Antisense | 1578 |
| 209795_at | 563 | 603 | 1315 | GACAGGTCCTTTTCGATGGTACATA | Antisense | 1579 |
| 209795_at | 188 | 907 | 1331 | TGGTACATATTTCTTTGCCTTTATA | Antisense | 1580 |
| 209795_at | 302 | 1083 | 1352 | TATAATCTTTTATACAGTGTCTTAC | Antisense | 1581 |
| 209795_at | 1015 | 773 | 1450 | GTGATGTGGCAAATCTCTATTAGGA | Antisense | 1582 |
| 209795_at | 970 | 21 | 1476 | ATATTCTGTAATCTTCAGACCTAGA | Antisense | 1583 |
| 209795_at | 859 | 83 | 1520 | AGGTTTGTGACTTTCCTAAATCAAT | Antisense | 1584 |
| 209795_at | 232 | 1059 | 1550 | TACGTGCAATACTTCAATACTTCAT | Antisense | 1585 |
| 210017_at | 352 | 275 | 4267 | AATGACTGCAAAAGTGCCACGGATA | Antisense | 1586 |
| 210017_at | 721 | 95 | 4279 | AGTGCCACGGATATCAATTTGAGGG | Antisense | 1587 |
| 210017_at | 1160 | 657 | 4355 | GAGGATCAACTGTACCATATTTAAT | Antisense | 1588 |
| 210017_at | 1097 | 267 | 4470 | AAGGAATCTTCCTAGATATGATAAA | Antisense | 1589 |
| 210017_at | 869 | 487 | 4512 | GCCCACACCTAACATTATACTTCAT | Antisense | 1590 |
| 210017_at | 914 | 613 | 4544 | GACTGAAGGCTGAATGTTTTCCCCT | Antisense | 1591 |
| 210017_at | 748 | 77 | 4583 | AGGACAAGGATGTTCACTCGGCACT | Antisense | 1592 |
| 210017_at | 249 | 995 | 4600 | TCGGCACTACTTCTATTCAGCATTG | Antisense | 1593 |
| 210017_at | 447 | 1055 | 4607 | TACTTCTATTCAGCATTGTACTTGA | Antisense | 1594 |
| 210017_at | 349 | 1007 | 4635 | TCTAGCCACAGCAGTTAGGTTAGGA | Antisense | 1595 |
| 210017_at | 1095 | 1073 | 4655 | TAGGAATTCAAGGTTTGTTCAACAT | Antisense | 1596 |
| 210052_s_at | 383 | 99 | 2683 | AGTCAAGTGACCAGCCTCTGACTGT | Antisense | 1597 |
| 210052_s_at | 605 | 171 | 2730 | ACTCGATTCCACTGCTAAACTCAGC | Antisense | 1598 |
| 210052_s_at | 375 | 1025 | 2750 | TCAGCTGTGAGCTGCGGATACCGCC | Antisense | 1599 |
| 210052_s_at | 916 | 155 | 2785 | ACCTGCTCTTAACCTCAAACCTAGG | Antisense | 1600 |
| 210052_s_at | 1037 | 369 | 2805 | CTAGGACCGTCTTGCTTTGTCATTG | Antisense | 1601 |
| 210052_s_at | 959 | 847 | 2835 | GGAGAGAACCCATTTCTCCAGACTT | Antisense | 1602 |
| 210052_s_at | 882 | 1115 | 2860 | TTACCTACCCGTGCCTGAGAAAGCA | Antisense | 1603 |
| 210052_s_at | 1069 | 521 | 2882 | GCATACTTGACAACTGTGGACTCCA | Antisense | 1604 |
| 210052_s_at | 935 | 1053 | 2991 | TACTTCCTTTAAACCATCAGCCGGC | Antisense | 1605 |
| 210052_s_at | 675 | 477 | 3014 | GCCTTTTATATGGGTCTTCACTCTG | Antisense | 1606 |
| 210052_s_at | 556 | 779 | 3067 | GTGTAATCTCTATTGCTATTGCCCC | Antisense | 1607 |
| 210258_at | 201 | 189 | 994 | ACAGCAAGCCTATGTAGTTCAATTA | Antisense | 1608 |
| 210258_at | 789 | 777 | 1167 | GTGTTTCTAGCATGAATGTTCTATA | Antisense | 1609 |
| 210258_at | 785 | 599 | 1219 | GACAAATGCTACTCACAGTACAATC | Antisense | 1610 |
| 210258_at | 84 | 819 | 1274 | GGTGTTCTTCAGAGACATTTTATCT | Antisense | 1611 |
| 210258_at | 538 | 199 | 1302 | AAATTTTCCTACTATTATGTTCATT | Antisense | 1612 |
| 210258_at | 638 | 227 | 1327 | AACAAACTTCTTTATCACATGTATC | Antisense | 1613 |
| 210258_at | 1135 | 719 | 1347 | GTATCTTCTACGTGTAAAACATTTC | Antisense | 1614 |
| 210258_at | 20 | 57 | 1396 | ATGAATTTCTTCATTTGCTCTTGCA | Antisense | 1615 |
| 210258_at | 407 | 1149 | 1410 | TTGCTCTTGCATCTACATTGCTATA | Antisense | 1616 |
| 210258_at | 239 | 61 | 1467 | ATGTTTTTTCCTTACAATGTGAACT | Antisense | 1617 |
| 210258_at | 142 | 295 | 1481 | CAATGTGAACTCATCGTGATCTTGG | Antisense | 1618 |
| 210260_s_at | 1031 | 693 | 1244 | GATTGAGTCATCGACATTCAGGATT | Antisense | 1619 |
| 210260_s_at | 234 | 1 | 1263 | AGGATTTAAGTCTGAGGTAGTCAAC | Antisense | 1620 |
| 210260_s_at | 946 | 219 | 1300 | AAAAATGGCTTATCTGAAATCAGTA | Antisense | 1621 |
| 210260_s_at | 544 | 55 | 1376 | ATGCTTCTGGAATTGAGTTCTCCTT | Antisense | 1622 |
| 210260_s_at | 142 | 647 | 1390 | GAGTTCTCCTTTTAAGTACCAATGA | Antisense | 1623 |
| 210260_s_at | 855 | 923 | 1434 | TGTAATGGTGTGTCATTGCCTTGAA | Antisense | 1624 |
| 210260_s_at | 897 | 779 | 1443 | GTGTCATTGCCTTGAAATGCTTGCT | Antisense | 1625 |
| 210260_s_at | 276 | 949 | 1455 | TGAAATGCTTGCTTAGGGCTTCTTT | Antisense | 1626 |
| 210260_s_at | 86 | 223 | 1546 | AAAAGTCTAGATTGGTCTTGATATT | Antisense | 1627 |
| 210260_s_at | 748 | 293 | 1607 | CAATCTTCATTCTACAGATGAACTC | Antisense | 1628 |
| 210260_s_at | 833 | 493 | 1701 | GCGTGCGTTTCAGTGTTTAAGAAGG | Antisense | 1629 |
| 210306_at | 923 | 269 | 2827 | AATGGGTTTAATGAGGTCTACCTTG | Antisense | 1630 |
| 210306_at | 1120 | 357 | 2848 | CTTGCAGAGCCATTGTGAGCATTGG | Antisense | 1631 |
| 210306_at | 273 | 553 | 2884 | GAATCATACCAGAACGTCTAGTATA | Antisense | 1632 |
| 210306_at | 319 | 313 | 2959 | CACTAGGCAATTCTGTCATTGTGTA | Antisense | 1633 |
| 210306_at | 951 | 1057 | 3079 | TACTGAATGTCAGTCATTTCCCCTA | Antisense | 1634 |
| 210306_at | 198 | 547 | 3112 | GCAATGCCAATATCAAGGGCCATGT | Antisense | 1635 |
| 210306_at | 366 | 263 | 3126 | AAGGGCCATGTATCAGGTTTCTGTA | Antisense | 1636 |
| 210306_at | 440 | 339 | 3139 | CAGGTTTCTGTATATGTTCCACTAT | Antisense | 1637 |
| 210306_at | 626 | 729 | 3218 | GTAGCATATGGCTGTGTATCACTAG | Antisense | 1638 |
| 210306_at | 586 | 1077 | 3271 | TATGTAGATCCAATCACTTTACCTA | Antisense | 1639 |
| 210306_at | 13 | 205 | 3300 | AAATGACTGCTATGGTGGGAACACA | Antisense | 1640 |
| 210976_s_at | 218 | 491 | 2479 | GCGTAAGAGGGCTCTGGTCTTCCAA | Antisense | 1641 |
| 210976_s_at | 106 | 1089 | 2534 | TTTGAGCATCGAATCCCCAAGGAAC | Antisense | 1642 |
| 210976_s_at | 359 | 237 | 2571 | AACTGAGGCCCATCCTCAAAATCCT | Antisense | 1643 |
| 210976_s_at | 614 | 947 | 2611 | TGACTTGGACACTTCAGACCATGCC | Antisense | 1644 |
| 210976_s_at | 667 | 969 | 2674 | TGCCGTCTAAACCTCTCTGGAGTGA | Antisense | 1645 |
| 210976_s_at | 1127 | 137 | 2725 | AGCTCACACCCTAATAAGTCCACAT | Antisense | 1646 |
| 210976_s_at | 972 | 261 | 2740 | AAGTCCACATCTTCTCAGTGTTTTA | Antisense | 1647 |
| 210976_s_at | 67 | 1089 | 2791 | TATTCTGTACCTTGCAGCCATGACC | Antisense | 1648 |
| 210976_s_at | 334 | 423 | 2890 | CCCAGCTTTATCTGTCACACAAGGC | Antisense | 1649 |
| 210976_s_at | 442 | 511 | 2929 | GCTACTGCTAGATATCACTTACTCA | Antisense | 1650 |
| 210976_s_at | 938 | 253 | 3002 | AAGAGTCTTGGTTCCTCTACTACTT | Antisense | 1651 |
| 211149_at | 115 | 327 | 6000 | CAGAAACCTCGATATATAATTGTAT | Antisense | 1652 |
| 211149_at | 70 | 1113 | 6039 | TTATTTTTTACATCTATGGTAGTTT | Antisense | 1653 |
| 211149_at | 365 | 1 | 6054 | ATGGTAGTTTTTGAGGTGCCTATTA | Antisense | 1654 |
| 211149_at | 515 | 1047 | 6079 | TAAAGTATTACGGAAGTTTGCTGTT | Antisense | 1655 |
| 211149_at | 157 | 689 | 6128 | GATTTATTAAGTTGTAGTCACCATA | Antisense | 1656 |
| 211149_at | 1129 | 727 | 6141 | GTAGTCACCATAGTGATAGCCCATA | Antisense | 1657 |
| 211149_at | 1159 | 683 | 6155 | GATAGCCCATAAATAATTGCTGGAA | Antisense | 1658 |
| 211149_at | 290 | 691 | 6294 | GATTCAGAATGATAACCTCTTTTAG | Antisense | 1659 |
| 211149_at | 517 | 595 | 6361 | GAAATGCTATACTGTCCTGCTTGTA | Antisense | 1660 |
| 211149_at | 993 | 165 | 6371 | ACTGTCCTGCTTGTACAACATGGTT | Antisense | 1661 |
| 211149_at | 1021 | 725 | 6383 | GTACAACATGGTTTGGGGTGAAGGG | Antisense | 1662 |
| 211421_s_at | 174 | 269 | 3054 | AAGGCTTGGATGCGTGTGTAATAGA | Antisense | 1663 |
| 211421_s_at | 162 | 29 | 3074 | ATAGAGCCTTATGGTGTGTGCGCAC | Antisense | 1664 |
| 211421_s_at | 1105 | 1089 | 3128 | TATTGGACACGTAACCTGGCTCTAA | Antisense | 1665 |
| 211421_s_at | 646 | 157 | 3141 | ACCTGGCTCTAATTTGGGCTGTTTT | Antisense | 1666 |
| 211421_s_at | 1160 | 681 | 3169 | GATACACTGTGATAAGTTCTTTTAC | Antisense | 1667 |
| 211421_s_at | 166 | 217 | 3213 | AAACTTTTGGTTTTCAGATATGCTT | Antisense | 1668 |
| 211421_s_at | 386 | 685 | 3229 | GATATGCTTAATGATAGTCTTACTA | Antisense | 1669 |
| 211421_s_at | 833 | 501 | 3317 | GCTGCAACAGGTTTGTTCTCAGGAG | Antisense | 1670 |
| 211421_s_at | 1144 | 623 | 3409 | GACCAATAACCTAATTGTCTATTCC | Antisense | 1671 |
| 211421_s_at | 724 | 987 | 3447 | TCCCCATCCTTATTAGCTCTACTGG | Antisense | 1672 |
| 211421_s_at | 797 | 243 | 3584 | AACCAGATCATGTTCCTTTTTTTGT | Antisense | 1673 |
| 211603_s_at | 522 | 821 | 1524 | GGTGCTGCCCTGTGTACATATAAAT | Antisense | 1674 |
| 211603_s_at | 740 | 817 | 1555 | GGTGTTGGGGAAACCTTCATCTGAA | Antisense | 1675 |
| 211603_s_at | 193 | 121 | 1634 | AGACTCTGAGCTGCTCACCGGAGTC | Antisense | 1676 |
| 211603_s_at | 960 | 501 | 1643 | GCTGCTCACCGGAGTCATTGGGAAG | Antisense | 1677 |
| 211603_s_at | 23 | 371 | 1690 | CTAGAGTCTCAGAAACTCCCCTGGG | Antisense | 1678 |
| 211603_s_at | 491 | 843 | 1732 | GGAGGAATTCAGCTCAGCTTCTTCC | Antisense | 1679 |
| 211603_s_at | 664 | 195 | 1797 | ACAAGAGTTTGTTCTGTTCTGGGGG | Antisense | 1680 |
| 211603_s_at | 444 | 995 | 1814 | TCTGGGGGACAGAGAAGGCGCTTCC | Antisense | 1681 |
| 211603_s_at | 991 | 515 | 1833 | GCTTCCCAACTTCATACTGGCAGGA | Antisense | 1682 |
| 211603_s_at | 540 | 819 | 1859 | GGTGAGGAGGTTCACTGAGCTCCCC | Antisense | 1683 |
| 211603_s_at | 718 | 421 | 1890 | CCCACTGCGGGGAGACAGAAGCCTG | Antisense | 1684 |
| 211676_s_at | 750 | 583 | 1111 | GAAAATATTCCTGACGTGGTCCCGG | Antisense | 1685 |
| 211676_s_at | 850 | 535 | 1136 | GCAGCCATCTGACTCCAATAGAGAG | Antisense | 1686 |
| 211676_s_at | 505 | 641 | 1158 | GAGAGAGAGTTCTTCACCTTTAAGT | Antisense | 1687 |
| 211676_s_at | 100 | 739 | 1184 | GTAACCAGTCTGAACCTGGCAGCAT | Antisense | 1688 |
| 211676_s_at | 510 | 873 | 1201 | GGCAGCATCGCTTTAAACTCGTATC | Antisense | 1689 |
| 211676_s_at | 697 | 215 | 1215 | AAACTCGTATCACTCCAGAAATTGT | Antisense | 1690 |
| 211676_s_at | 1114 | 1091 | 1267 | TTTGATACTGATTCCAGCTGTCTGG | Antisense | 1691 |
| 211676_s_at | 594 | 553 | 1291 | GAATCACATAGCTCCTTATCTGACT | Antisense | 1692 |
| 211676_s_at | 936 | 1115 | 1306 | TTATCTGACTCAGAATTTCCCCCAA | Antisense | 1693 |
| 211676_s_at | 995 | 559 | 1354 | GAAGGACAAGAGCTCATAACCGTAA | Antisense | 1694 |
| 211676_s_at | 855 | 183 | 1413 | ACATGTGCTAGTGGATCTACTTGTG | Antisense | 1695 |
| 212134_at | 150 | 1025 | 4469 | TCAGCCCTGATGCACAGGCTGCCAG | Antisense | 1696 |
| 212134_at | 502 | 165 | 4520 | ACTGGTGCCTTGCTTAGAGCCAGAA | Antisense | 1697 |
| 212134_at | 749 | 469 | 4538 | GCCAGAAGGGATGAAGCCGGGGGAT | Antisense | 1698 |
| 212134_at | 1101 | 571 | 4550 | GAAGCCGGGGGATCTATGGAACAGA | Antisense | 1699 |
| 212134_at | 726 | 659 | 4573 | GAGGAGGAGCGATGCAGTTGGGAGA | Antisense | 1700 |
| 212134_at | 868 | 861 | 4598 | GGAAGCTAGAAGGGTTATGGTTGGA | Antisense | 1701 |
| 212134_at | 254 | 331 | 4708 | CAGAGGGGAAGGGCCCTGTGGTGCC | Antisense | 1702 |
| 212134_at | 119 | 1145 | 4740 | TTCCTTCAGCCTCCGAAGGGTGATG | Antisense | 1703 |
| 212134_at | 613 | 519 | 4855 | GCATTGAGCCCTTGGTTGCCTGGGC | Antisense | 1704 |
| 212134_at | 383 | 811 | 4887 | GGGGGTTTTCAGTATTTGTAAGCAT | Antisense | 1705 |
| 212134_at | 1015 | 721 | 4898 | GTATTTGTAAGCATTTCAGCAGAAC | Antisense | 1706 |
| 212349_at | 697 | 903 | 4080 | TGGAGTGTGTTCCTGAAGAGCAGCC | Antisense | 1707 |
| 212349_at | 103 | 663 | 4125 | GAGGCAGGCATAGGCAGGGAACCGA | Antisense | 1708 |
| 212349_at | 156 | 799 | 4141 | GGGAACCGAGCAGCAGGTCAGAGCA | Antisense | 1709 |
| 212349_at | 1027 | 73 | 4165 | AGGCGAGCTGACATTCTGCAGCCTG | Antisense | 1710 |
| 212349_at | 733 | 481 | 4185 | GCCTGGACGGCCATGGCAGGAAGCT | Antisense | 1711 |
| 212349_at | 913 | 245 | 4230 | AAGCCTCCTAGGGTTCTGAGCAGAA | Antisense | 1712 |
| 212349_at | 1123 | 813 | 4257 | GGGGCATGAGCTGATTCACATTCTG | Antisense | 1713 |
| 212349_at | 955 | 179 | 4274 | ACATTCTGAAGGACCTCTCTAGCTG | Antisense | 1714 |
| 212349_at | 236 | 211 | 4333 | AAAGCAGAGAGACCAGTGCAGGGCT | Antisense | 1715 |
| 212349_at | 790 | 699 | 4367 | GTTGCAGGCGAGAGACTGGGGTGCT | Antisense | 1716 |
| 212349_at | 173 | 1005 | 4425 | TCTCCCAAGAGACAAAGGCCATTGC | Antisense | 1717 |
| 212379_at | 732 | 201 | 2003 | AAATCGTGGCAAAATCTTCCCTCCA | Antisense | 1718 |
| 212379_at | 36 | 553 | 2064 | GACTTTAGGGGACTTACTTCTCACG | Antisense | 1719 |
| 212379_at | 1023 | 553 | 2096 | GAATCTACAGCCATTCACTGTTACC | Antisense | 1720 |
| 212379_at | 397 | 313 | 2111 | CACTGTTACCTGTCCTACGTTCAGG | Antisense | 1721 |
| 212379_at | 501 | 243 | 2144 | AAGCCTTTGCCCATATTACTGGTGG | Antisense | 1722 |
| 212379_at | 795 | 173 | 2175 | ACTAGAGAACATCCCCAGAGTCCTC | Antisense | 1723 |
| 212379_at | 22 | 621 | 2232 | GACCTGGAGGATCCCCAGGGTCTTC | Antisense | 1724 |
| 212379_at | 1021 | 325 | 2266 | CAGCAGGAAGGACACCTCTCTGAGG | Antisense | 1725 |
| 212379_at | 171 | 787 | 2413 | GTGGTTGCACGAGCTGAAGGTTCCC | Antisense | 1726 |
| 212379_at | 405 | 557 | 2428 | GAAGGTTCCCCACGTGTGAAAGTCA | Antisense | 1727 |
| 212379_at | 1000 | 557 | 2496 | GAATGGCTCCCTGACAAATCATTTC | Antisense | 1728 |
| 212654_at | 304 | 815 | 804 | GGTGGCCGAGAGTAAATGTGGGGAC | Antisense | 1729 |
| 212654_at | 339 | 737 | 815 | GTAAATGTGGGGACCTAGAGGAGGA | Antisense | 1730 |
| 212654_at | 180 | 903 | 872 | TGGAGGCCCAGGCGGACAAGTATTC | Antisense | 1731 |
| 212654_at | 145 | 341 | 880 | CAGGCGGACAAGTATTCCACCAAAG | Antisense | 1732 |
| 212654_at | 339 | 673 | 1024 | GATGAAGTCTATGCCCAGAAGATGA | Antisense | 1733 |
| 212654_at | 447 | 77 | 1067 | AGGAACTGGACAACGCACTCAATGA | Antisense | 1734 |
| 212654_at | 592 | 599 | 1075 | GACAACGCACTCAATGACATCACCT | Antisense | 1735 |
| 212654_at | 541 | 981 | 1149 | TCCTTTCCATTCTCTCTATGGGGAG | Antisense | 1736 |
| 212654_at | 13 | 661 | 1186 | GAGGAGCAGAAATTGCCAACATTGC | Antisense | 1737 |
| 212654_at | 183 | 1147 | 1198 | TTGCCAACATTGCACAGCCAGGCTG | Antisense | 1738 |
| 212654_at | 208 | 405 | 1220 | CTGGGAGCAGCCTAGGGAGAGCCCC | Antisense | 1739 |
| 212671_s_at | 689 | 149 | 439 | ACCAATGAGGTTCCTGAGGTCACAG | Antisense | 1740 |
| 212671_s_at | 593 | 59 | 443 | ATGAGGTTCCTGAGGTCACAGTGTT | Antisense | 1741 |
| 212671_s_at | 710 | 83 | 446 | AGGTTCCTGAGGTCACAGTGTTTTC | Antisense | 1742 |
| 212671_s_at | 604 | 983 | 450 | TCCTGAGGTCACAGTGTTTTCCAAG | Antisense | 1743 |
| 212671_s_at | 458 | 749 | 512 | GTCTTGTGGACAACATCTTTCCTCC | Antisense | 1744 |
| 212671_s_at | 615 | 383 | 604 | CTCTCCAAGAGTGATCATTCCTTCT | Antisense | 1745 |
| 212671_s_at | 807 | 467 | 608 | CCAAGAGTGATCATTCCTTCTTCAA | Antisense | 1746 |
| 212671_s_at | 232 | 113 | 611 | AGAGTGATCATTCCTTCTTCAAGAT | Antisense | 1747 |
| 212671_s_at | 793 | 93 | 613 | AGTGATCATTCCTTCTTCAAGATCA | Antisense | 1748 |
| 212671_s_at | 731 | 671 | 664 | GATGAGATTTATGACTGCAAGGTGG | Antisense | 1749 |
| 212671_s_at | 573 | 443 | 709 | CCTCTTCTGAAACACTGGGAGCCTG | Antisense | 1750 |
| 212684_at | 268 | 611 | 1158 | GACTATTTCAAAGCTACTGTTCCTA | Antisense | 1751 |
| 212684_at | 555 | 699 | 1176 | GTTCCTAGTCCAGCTTTAAGTTTCG | Antisense | 1752 |
| 212684_at | 683 | 499 | 1212 | GCTGTTTTGTTTCATGATTTCGTTA | Antisense | 1753 |
| 212684_at | 414 | 671 | 1305 | GATGATGGTGTCCTTTAGGGCTCTT | Antisense | 1754 |
| 212684_at | 1067 | 89 | 1321 | AGGGCTCTTGGAGCAGCCAGACCAT | Antisense | 1755 |
| 212684_at | 957 | 103 | 1399 | AGTTGTCCTGGGGCTGAATGGGCAA | Antisense | 1756 |
| 212684_at | 1098 | 557 | 1414 | GAATGGGCAAATCTGTCCAAACAGC | Antisense | 1757 |
| 212684_at | 596 | 495 | 1437 | GCTAGTAACCGGCTGTGAGGGAGAG | Antisense | 1758 |
| 212684_at | 844 | 745 | 1463 | GTCAGAAGCACTTAGCGTTGGCCTC | Antisense | 1759 |
| 212684_at | 377 | 61 | 1532 | ATGATTTTCTCTAAATGCCTGGGTA | Antisense | 1760 |
| 212684_at | 148 | 1127 | 1565 | TTCAAGGAGCTCACTTGGCCTGCTT | Antisense | 1761 |
| 212775_at | 944 | 1025 | 2521 | TCACCGTCACAGAGTCTTACCAAAG | Antisense | 1762 |
| 212775_at | 556 | 743 | 2526 | GTCACAGAGTCTTACCAAAGTCAGG | Antisense | 1763 |
| 212775_at | 951 | 305 | 2541 | CAAAGTCAGGACAGTTCAAATAACA | Antisense | 1764 |
| 212775_at | 996 | 225 | 2562 | AACAATCCGGAGTTATGCGTCCTCT | Antisense | 1765 |
| 212775_at | 1056 | 293 | 2564 | CAATCCGGAGTTATGCGTCCTCTTG | Antisense | 1766 |
| 212775_at | 787 | 985 | 2567 | TCCGGAGTTATGCGTCCTCTTGAAA | Antisense | 1767 |
| 212775_at | 846 | 709 | 2573 | GTTATGCGTCCTCTTGAAAAAGCCG | Antisense | 1768 |
| 212775_at | 209 | 493 | 2578 | GCGTCCTCTTGAAAAAGCCGAAGAC | Antisense | 1769 |
| 212775_at | 1078 | 455 | 2628 | CCATGGCGACGAACAGCTGGCACTG | Antisense | 1770 |
| 212775_at | 66 | 417 | 2637 | CGAACAGCTGGCACTGAGTAGCAGC | Antisense | 1771 |
| 212775_at | 720 | 189 | 2640 | ACAGCTGGCACTGAGTAGCAGCTGC | Antisense | 1772 |
| 212776_s_at | 63 | 1003 | 2263 | TCTCCTCGTGGATCGTGTATCCCAG | Antisense | 1773 |
| 212776_s_at | 507 | 983 | 2265 | TCCTCGTGGATCGTGTATCCCAGCG | Antisense | 1774 |
| 212776_s_at | 1108 | 411 | 2276 | CGTGTATCCCAGCGGCAAGGTGTAT | Antisense | 1775 |
| 212776_s_at | 210 | 723 | 2279 | GTATCCCAGCGGCAAGGTGTATGTG | Antisense | 1776 |
| 212776_s_at | 163 | 461 | 2284 | CCAGCGGCAAGGTGTATGTGGCAGC | Antisense | 1777 |
| 212776_s_at | 1127 | 915 | 2324 | TGTGGTGCTGACCTGTGAGCTATGC | Antisense | 1778 |
| 212776_s_at | 404 | 531 | 2358 | GCAGAGGTGCGCTGGACCAAGGATG | Antisense | 1779 |
| 212776_s_at | 4 | 129 | 2461 | AGCTCGAGGACTCCGGCGAGTACTT | Antisense | 1780 |
| 212776_s_at | 921 | 411 | 2465 | CGAGGACTCCGGCGAGTACTTGTGT | Antisense | 1781 |
| 212776_s_at | 512 | 613 | 2469 | GACTCCGGCGAGTACTTGTGTGAAA | Antisense | 1782 |
| 212776_s_at | 362 | 197 | 2491 | AAATTGACGATGAGTCGGCCTCCTT | Antisense | 1783 |
| 212801_at | 618 | 295 | 5406 | CAAGGCATCTGTTGCTTTGGGTCCT | Antisense | 1784 |
| 212801_at | 713 | 945 | 5517 | TGACCGTGTTTTCAACTGTGTCCAC | Antisense | 1785 |
| 212801_at | 281 | 977 | 5556 | TCCAGATTGCAACCTGGCCTGCATC | Antisense | 1786 |
| 212801_at | 281 | 983 | 5590 | TCCTGCTCGTGTCTTAACCTAAGTG | Antisense | 1787 |
| 212801_at | 589 | 389 | 5641 | CTCCATGTGGTAGCTCCTTTGGCAA | Antisense | 1788 |
| 212801_at | 65 | 273 | 5665 | AATGTCCTGCTGTGGCGTTTTATGT | Antisense | 1789 |
| 212801_at | 343 | 65 | 5686 | ATGTGTTGCTTGGAGTCTGTGGGGT | Antisense | 1790 |
| 212801_at | 1031 | 987 | 5725 | TCCCGTCCCCAGGGCAGATTTGATT | Antisense | 1791 |
| 212801_at | 674 | 499 | 5758 | GCTGAAGTTTTGTCTCTTGGTCCAC | Antisense | 1792 |
| 212801_at | 714 | 271 | 5805 | AATGGGTCTTTCAGTCTTGGCATTT | Antisense | 1793 |
| 212801_at | 1139 | 721 | 5875 | GTATATTGTGTGTCTCATCTGTGAA | Antisense | 1794 |
| 212877_at | 1091 | 657 | 411 | GAGGAAAATGAAGCTCGGGCTGGTT | Antisense | 1795 |
| 212877_at | 973 | 571 | 420 | GAAGCTCGGGCTGGTTAACTGACTT | Antisense | 1796 |
| 212877_at | 178 | 505 | 423 | GCTCGGGCTGGTTAACTGACTTGCT | Antisense | 1797 |
| 212877_at | 519 | 407 | 426 | CGGGCTGGTTAACTGACTTGCTCAG | Antisense | 1798 |
| 212877_at | 244 | 497 | 429 | GCTGGTTAACTGACTTGCTCAGCGT | Antisense | 1799 |
| 212877_at | 613 | 417 | 543 | CGCTGTGTCTAGCAGCCTCTAGGAT | Antisense | 1800 |
| 212877_at | 800 | 923 | 548 | TGTCTAGCAGCCTCTAGGATCTTGT | Antisense | 1801 |
| 212877_at | 607 | 441 | 637 | CCTGATTTCTGTATACATGTAGCTT | Antisense | 1802 |
| 212877_at | 713 | 927 | 646 | TGTATACATGTAGCTTTGCCAGATA | Antisense | 1803 |
| 212877_at | 752 | 137 | 657 | AGCTTTGCCAGATATGTACTTAGTA | Antisense | 1804 |
| 212877_at | 1159 | 1083 | 692 | TATTAATAAAATCCATTTACTGTGT | Antisense | 1805 |
| 213077_at | 465 | 185 | 1831 | ACAGGTTTGATGGCACTTCTCATGA | Antisense | 1806 |
| 213077_at | 84 | 873 | 1842 | GGCACTTCTCATGATACATTTTAGT | Antisense | 1807 |
| 213077_at | 20 | 201 | 1970 | AAATCTTGAGAACCTTCTACTTTCT | Antisense | 1808 |
| 213077_at | 244 | 593 | 2029 | GAAAGTCAGCTATGATTTTGCCTAT | Antisense | 1809 |
| 213077_at | 768 | 689 | 2042 | GATTTTGCCTATAGTTCTAGTTATT | Antisense | 1810 |
| 213077_at | 580 | 607 | 2093 | GACATACCTGTAAATTGAACCTATT | Antisense | 1811 |
| 213077_at | 285 | 401 | 2132 | CTGTATGTGTATTATGGCTCTTTTC | Antisense | 1812 |
| 213077_at | 263 | 71 | 2145 | ATGGCTCTTTTCCTATTAGAGCAAC | Antisense | 1813 |
| 213077_at | 485 | 1067 | 2161 | TAGAGCAACTTGTGTTTCCCTGATA | Antisense | 1814 |
| 213077_at | 486 | 777 | 2172 | GTGTTTCCCTGATAATGTGTACATT | Antisense | 1815 |
| 213077_at | 779 | 399 | 2274 | CTGTTTTGTAATATCTAGCTCTATA | Antisense | 1816 |
| 213196_at | 648 | 387 | 5484 | CTCCCCAAGCTCCTTTAACCAGGAG | Antisense | 1817 |
| 213196_at | 1044 | 651 | 5520 | GAGTGAGCTACGTCTTGGGAGGATT | Antisense | 1818 |
| 213196_at | 72 | 881 | 5564 | GGCTAGAGTTGCTCAGATCGGCTCA | Antisense | 1819 |
| 213196_at | 592 | 267 | 5593 | AAGGCCCAGGGGTCAAAGCATTTGC | Antisense | 1820 |
| 213196_at | 507 | 521 | 5610 | GCATTTGCTCACCACAACTGAGTCT | Antisense | 1821 |
| 213196_at | 166 | 431 | 5687 | CCCCTCAGAAACGCCCTGGTGGAGG | Antisense | 1822 |
| 213196_at | 946 | 797 | 5843 | GGGAAACTTGAAGCCATCACAGCCA | Antisense | 1823 |
| 213196_at | 1059 | 979 | 5914 | TCCTTTGCCCTCACTAGGGAAGCCC | Antisense | 1824 |
| 213196_at | 965 | 409 | 5946 | CGGACTTGTCCCCATGGAGTGAAGA | Antisense | 1825 |
| 213196_at | 205 | 769 | 5964 | GTGAAGAGAGGCCCCATTTTTGAGT | Antisense | 1826 |
| 213196_at | 767 | 697 | 6008 | GTTGTGTCACTGGTGGTCATGTTGC | Antisense | 1827 |
| 213295_at | 517 | 903 | 2161 | TGGAGGGATTGTCCTTTCAAGCACC | Antisense | 1828 |
| 213295_at | 117 | 981 | 2172 | TCCTTTCAAGCACCACAGCTTCAGA | Antisense | 1829 |
| 213295_at | 225 | 735 | 2205 | GTACTTTCAAATATTGTCCACTTTA | Antisense | 1830 |
| 213295_at | 485 | 21 | 2253 | ATATTGGAGACTCAACTGCCCTTGG | Antisense | 1831 |
| 213295_at | 104 | 271 | 2291 | AATGGCCTAGTACTGTGGAATTTTA | Antisense | 1832 |
| 213295_at | 178 | 211 | 2356 | AAAGAACTCACATCCCATCTGAAAC | Antisense | 1833 |
| 213295_at | 220 | 347 | 2366 | CATCCCATCTGAAACTTCCCAGGGG | Antisense | 1834 |
| 213295_at | 102 | 991 | 2442 | TCCCATTTTTGGTATCTTGTTCAGC | Antisense | 1835 |
| 213295_at | 329 | 19 | 2480 | ATTTCTTGTCTGCAGAACATCCTAT | Antisense | 1836 |
| 213295_at | 139 | 253 | 2527 | AAGACCACCACATAGAATACCCCTT | Antisense | 1837 |
| 213295_at | 358 | 325 | 2557 | CAGCTCGCTCTGATTTAGCCTTAAT | Antisense | 1838 |
| 213547_at | 386 | 905 | 3651 | TGGTTCCTTCAGAGGGTGTCTCTGC | Antisense | 1839 |
| 213547_at | 806 | 1011 | 3755 | TCTTCACTGCATTCAGCAATGCCTG | Antisense | 1840 |
| 213547_at | 147 | 799 | 3937 | GGGAACTGGACAGGCTTGGACCTCA | Antisense | 1841 |
| 213547_at | 614 | 519 | 3950 | GCTTGGACCTCATGTTTCATTTCTA | Antisense | 1842 |
| 213547_at | 1103 | 1 | 3997 | ATTGGGCAACAATGGGCATCTTCCA | Antisense | 1843 |
| 213547_at | 248 | 227 | 4004 | AACAATGGGCATCTTCCATGCCACC | Antisense | 1844 |
| 213547_at | 878 | 461 | 4024 | CCACCACCCAGGCATAACCAGTTGG | Antisense | 1845 |
| 213547_at | 153 | 265 | 4066 | AAGGTTTCAAATGTGTCTAGTGTTC | Antisense | 1846 |
| 213547_at | 371 | 65 | 4076 | ATGTGTCTAGTGTTCAGTATTGAGG | Antisense | 1847 |
| 213547_at | 28 | 877 | 4117 | GGCAGGCCCAAGTATTTTCTGTGAT | Antisense | 1848 |
| 213547_at | 450 | 721 | 4128 | GTATTTTCTGTGATATCCCAGGTTA | Antisense | 1849 |
| 213551_x_at | 572 | 657 | 170 | GAGGTCTCAGCTCTTGGAGGAGGGC | Antisense | 1850 |
| 213551_x_at | 674 | 241 | 250 | AACCCTCACTAGCAGGACTGGTGGT | Antisense | 1851 |
| 213551_x_at | 828 | 751 | 278 | GTCTCACCTGGGGCCTAGAGTGGAA | Antisense | 1852 |
| 213551_x_at | 713 | 809 | 307 | GGGTGGGTTAACCTCACACAAGCAC | Antisense | 1853 |
| 213551_x_at | 42 | 299 | 325 | CAAGCACAGATCCCAGACTTTGCCA | Antisense | 1854 |
| 213551_x_at | 211 | 1073 | 504 | TAGGGACAGACCCTGGCAGGTCGCT | Antisense | 1855 |
| 213551_x_at | 846 | 421 | 530 | CCCACCGAGATTTCCTCAAGTGTGC | Antisense | 1856 |
| 213551_x_at | 32 | 639 | 580 | GAGACTCGAGATTCCATCATAGCGT | Antisense | 1857 |
| 213551_x_at | 1160 | 427 | 626 | CCCTGATGGGCTTGTCTGTGTTTGC | Antisense | 1858 |
| 213551_x_at | 990 | 779 | 661 | GTGTCTGAGGTCCTGTGACTGTACC | Antisense | 1859 |
| 213551_x_at | 110 | 981 | 687 | TCCTTTGCCCTGGGACATCTGTATC | Antisense | 1860 |
| 213569_at | 952 | 369 | 587 | CTAGGCTTGCTTTATCATCACATCC | Antisense | 1861 |
| 213569_at | 738 | 551 | 702 | GAATATGCAGAGGCCTTGGAGGAAG | Antisense | 1862 |
| 213569_at | 630 | 897 | 732 | TGGCAGTTATGACAGGAAGGCTCTC | Antisense | 1863 |
| 213569_at | 873 | 485 | 780 | GCCCCTGGCACTGAGCATGAGGAGC | Antisense | 1864 |
| 213569_at | 676 | 909 | 812 | TGGGGAGACTTTGCAATCACCCCCC | Antisense | 1865 |
| 213569_at | 1040 | 759 | 844 | GTCCATTTTCCACAGGTAGCTTTCT | Antisense | 1866 |
| 213569_at | 388 | 827 | 858 | GGTAGCTTTCTTGAACTCACCTTGA | Antisense | 1867 |
| 213569_at | 9 | 851 | 921 | GGAGCTGAAAGACAGTCCTGAACTG | Antisense | 1868 |
| 213569_at | 519 | 577 | 940 | GAACTGGGGGGAGCTGGGATCACAT | Antisense | 1869 |
| 213569_at | 94 | 487 | 972 | GCCCTGTCCCTCACAGGAAGTGAGA | Antisense | 1870 |
| 213569_at | 1161 | 905 | 1014 | TGGTGACTAAGGCCCCAAAGTCCCT | Antisense | 1871 |
| 213587_s_at | 569 | 87 | 208 | AGGGGAAGAAACGACAGCCTCACTT | Antisense | 1872 |
| 213587_s_at | 550 | 589 | 215 | GAAACGACAGCCTCACTTCTGTATG | Antisense | 1873 |
| 213587_s_at | 629 | 321 | 222 | CAGCCTCACTTCTGTATGGACTGCT | Antisense | 1874 |
| 213587_s_at | 784 | 999 | 232 | TCTGTATGGACTGCTGATGTGGCCT | Antisense | 1875 |
| 213587_s_at | 610 | 441 | 262 | CCTGTTCAGCGGGCATTGTCTTTGG | Antisense | 1876 |
| 213587_s_at | 3 | 1025 | 267 | TCAGCGGGCATTGTCTTTGGAGCAG | Antisense | 1877 |
| 213587_s_at | 149 | 879 | 273 | GGCATTGTCTTTGGAGCAGCAGGAG | Antisense | 1878 |
| 213587_s_at | 835 | 443 | 413 | CCTCCTGTTGGGTAAGGGTGTTGAG | Antisense | 1879 |
| 213587_s_at | 77 | 811 | 428 | GGGTGTTGAGTGTGACTTGTGCTGA | Antisense | 1880 |
| 213587_s_at | 296 | 939 | 434 | TGAGTGTGACTTGTGCTGAAAACCT | Antisense | 1881 |
| 213587_s_at | 418 | 753 | 446 | GTGCTGAAAACCTGGTTCATATATA | Antisense | 1882 |
| 213618_at | 96 | 295 | 4506 | CAATCTATATTCACAGGCCCATACT | Antisense | 1883 |
| 213618_at | 996 | 423 | 4523 | CCCATACTTCAGTCAGTCCAATCAT | Antisense | 1884 |
| 213618_at | 143 | 335 | 4532 | CAGTCAGTCCAATCATAGTACAGTG | Antisense | 1885 |
| 213618_at | 401 | 285 | 4624 | AATAGTTCACCATGGGTATGAGATA | Antisense | 1886 |
| 213618_at | 608 | 789 | 4719 | GTGGCACTATTAGGGAATCTTCCTT | Antisense | 1887 |
| 213618_at | 193 | 41 | 4877 | ATCTAGAGTTTTTACCTGCCTTATC | Antisense | 1888 |
| 213618_at | 878 | 1061 | 4889 | TACCTGCCTTATCTGAATTCTTCTT | Antisense | 1889 |
| 213618_at | 539 | 549 | 4903 | GAATTCTTCTTGAAACTTGAGCTTA | Antisense | 1890 |
| 213618_at | 303 | 1047 | 4926 | TAAACTCTAATAGCTGTTTCCCTTT | Antisense | 1891 |
| 213618_at | 56 | 1099 | 4942 | TTTCCCTTTCTATTCTGAACAACTG | Antisense | 1892 |
| 213618_at | 332 | 395 | 4956 | CTGAACAACTGTCTCCATTTTTCAA | Antisense | 1893 |
| 214131_at | 1071 | 1089 | 3000 | TTTGATGGGGACAGCCCACGTCTGA | Antisense | 1894 |
| 214131_at | 333 | 857 | 3008 | GGACAGCCCACGTCTGATAGGGTGG | Antisense | 1895 |
| 214131_at | 426 | 113 | 3050 | AGAGGGTTGTGCTCTTTCTAGTGTG | Antisense | 1896 |
| 214131_at | 1094 | 109 | 3087 | AGATGATATGTTGTAGCCACTTCTG | Antisense | 1897 |
| 214131_at | 883 | 463 | 3103 | CCACTTCTGCAGTGACGGGGACAGA | Antisense | 1898 |
| 214131_at | 698 | 649 | 3130 | GAGTCAGTGTTCTTAACTACAGTGG | Antisense | 1899 |
| 214131_at | 631 | 17 | 3218 | ATTTATTTCTTTGGTTAGTGGTGCA | Antisense | 1900 |
| 214131_at | 649 | 785 | 3235 | GTGGTGCAAGAAATTCTGTTTTCCT | Antisense | 1901 |
| 214131_at | 551 | 229 | 3301 | AACAGCTTGTAGATGCCAGACTTCA | Antisense | 1902 |
| 214131_at | 23 | 473 | 3315 | GCCAGACTTCAACAAACAGCACAGC | Antisense | 1903 |
| 214131_at | 509 | 639 | 3374 | GAGAGAGTTTGTAAGTTCTACTTCT | Antisense | 1904 |
| 214218_s_at | 949 | 761 | 423 | GTGCCAGACTTCTGAGAAGCACCTG | Antisense | 1905 |
| 214218_s_at | 173 | 303 | 452 | CAACAGCTTCCTTCTTTGAGCTTAG | Antisense | 1906 |
| 214218_s_at | 228 | 937 | 525 | TGATGACCAACTAATTCCCAAACCC | Antisense | 1907 |
| 214218_s_at | 957 | 469 | 592 | GCCACAATTGGTTTTGAAGTGCATT | Antisense | 1908 |
| 214218_s_at | 781 | 897 | 627 | TGGCATCACTACCACTACTGATTAA | Antisense | 1909 |
| 214218_s_at | 1060 | 1017 | 677 | TCATCTGCTTTATTCACATAAATGA | Antisense | 1910 |
| 214218_s_at | 631 | 289 | 741 | AATTAAGTGGCTTCGTCATTGTCCT | Antisense | 1911 |
| 214218_s_at | 1041 | 919 | 760 | TGTCCTTCTACCTCAAAGATAATTT | Antisense | 1912 |
| 214218_s_at | 743 | 771 | 898 | GTGACCACCAGAAAGTAATCTTAAG | Antisense | 1913 |
| 214218_s_at | 564 | 741 | 912 | GTAATCTTAAGCCATCTAGATGTCA | Antisense | 1914 |
| 214218_s_at | 938 | 473 | 922 | GCCATCTAGATGTCACAATTGAAAC | Antisense | 1915 |
| 214901_at | 123 | 415 | 1597 | CGAAGCCGGCGGCGTGAACAATCCT | Antisense | 1916 |
| 214901_at | 645 | 339 | 1626 | CAGGAACTCACACCTGGTTCAGCAT | Antisense | 1917 |
| 214901_at | 712 | 525 | 1678 | GCAGGCGGAGCAAAGGCAGGGCAGC | Antisense | 1918 |
| 214901_at | 919 | 865 | 1779 | GGAAGAGCCTTCTGTGGGTGCTTCC | Antisense | 1919 |
| 214901_at | 777 | 1019 | 1928 | TCATGGGTGACTTCTGACTTTCTAA | Antisense | 1920 |
| 214901_at | 234 | 359 | 1963 | CTTCCCAAGCACCCGAGGTTGGTTG | Antisense | 1921 |
| 214901_at | 549 | 833 | 1983 | GGTTGGTCCCAAATCTATCAAACTC | Antisense | 1922 |
| 214901_at | 848 | 487 | 2011 | GCCCTCTTTAGCGACATATTTTGTG | Antisense | 1923 |
| 214901_at | 724 | 1157 | 2031 | TTGTGACATTCCTTCCATTACACCA | Antisense | 1924 |
| 214901_at | 1017 | 31 | 2075 | ATAACCTACCCACCTGTGTAATGTC | Antisense | 1925 |
| 214901_at | 881 | 281 | 2106 | AATCAATATGCGGCCCCATTTTGTA | Antisense | 1926 |
| 214983_at | 802 | 701 | 4705 | GTTCCTTTTCGATTCTTGGTCTTAT | Antisense | 1927 |
| 214983_at | 1117 | 17 | 4728 | ATTTCTTCACCCATCTACTATAGTG | Antisense | 1928 |
| 214983_at | 437 | 1083 | 4746 | TATAGTGCATTCTTGCCATGCAGCC | Antisense | 1929 |
| 214983_at | 653 | 473 | 4760 | GCCATGCAGCCCTTTTTATGGAGCA | Antisense | 1930 |
| 214983_at | 238 | 189 | 4794 | ACAGCAGCTTGCTTCATTAGGGCAA | Antisense | 1931 |
| 214983_at | 221 | 843 | 4961 | GGAGGCTGCATCATGGGGAGCCATT | Antisense | 1932 |
| 214983_at | 985 | 801 | 4976 | GGGAGCCATTTTACAAGCAGCACCA | Antisense | 1933 |
| 214983_at | 390 | 1121 | 5022 | TTACATTTGTTCTGCTCTTAAGAGT | Antisense | 1934 |
| 214983_at | 110 | 273 | 5061 | AATGTGTAAGTCTGTCGTTTGTTCT | Antisense | 1935 |
| 214983_at | 707 | 755 | 5074 | GTCGTTTGTTCTTGACTTCTGTCAT | Antisense | 1936 |
| 214983_at | 719 | 277 | 5181 | AATCTCAGTTCATCAACGAATCGGT | Antisense | 1937 |
| 215314_at | 666 | 377 | 872 | CTCAGCCTCCTAAGGAGCTATTTTT | Antisense | 1938 |
| 215314_at | 661 | 763 | 1015 | GTGCCCAGCTAGATTCTTTACTTTT | Antisense | 1939 |
| 215314_at | 553 | 1079 | 1110 | TATGCCTCTTTCTGTAGATCAAATG | Antisense | 1940 |
| 215314_at | 783 | 595 | 1134 | GAAATATGGCCCAATTTACAAGGGT | Antisense | 1941 |
| 215314_at | 540 | 807 | 1155 | GGGTATACACCTTCATGGTAGATGA | Antisense | 1942 |
| 215314_at | 268 | 689 | 1217 | GATTTAATTGTAGTTTCTGCTGCCA | Antisense | 1943 |
| 215314_at | 1142 | 467 | 1238 | GCCAAAAAGTCTCCATAATTATTTT | Antisense | 1944 |
| 215314_at | 236 | 283 | 1265 | AATAACTTATAACTACCTTGATCTT | Antisense | 1945 |
| 215314_at | 1008 | 1041 | 1274 | TAACTACCTTGATCTTTACCTGTAC | Antisense | 1946 |
| 215314_at | 392 | 453 | 1280 | CCTTGATCTTTACCTGTACACACAC | Antisense | 1947 |
| 215314_at | 598 | 741 | 1330 | GTAATTTCACTTAGAATCCTGGTCA | Antisense | 1948 |
| 215359_x_at | 302 | 857 | 603 | GGACTCACACTGGAGAAAGACCCTA | Antisense | 1949 |
| 215359_x_at | 561 | 253 | 619 | AAGACCCTATGAATGTAAACACTGT | Antisense | 1950 |
| 215359_x_at | 159 | 97 | 656 | AGTCGTTCCAGTTTCTGTCGAGAAC | Antisense | 1951 |
| 215359_x_at | 179 | 917 | 726 | TGTGGGAAAGCCTTCAGTTCTCTCA | Antisense | 1952 |
| 215359_x_at | 908 | 1129 | 738 | TTCAGTTCTCTCAGTTCCTTTAATA | Antisense | 1953 |
| 215359_x_at | 1092 | 519 | 817 | GCATTCATTGGTTTTATCACATTCA | Antisense | 1954 |
| 215359_x_at | 885 | 199 | 858 | AAATCCTGTGAATGTAAACGTGGTA | Antisense | 1955 |
| 215359_x_at | 574 | 251 | 890 | AAGAAGTTTCCAGGCTGGGCGCAGC | Antisense | 1956 |
| 215359_x_at | 761 | 483 | 947 | GCCGAGGAGGGCAGATCACGAGGCC | Antisense | 1957 |
| 215359_x_at | 908 | 1063 | 1043 | TAGCCAGGCATAGTTGCTCACACCT | Antisense | 1958 |
| 215359_x_at | 860 | 75 | 1048 | AGGCATAGTTGCTCACACCTGTAGT | Antisense | 1959 |
| 215666_at | 26 | 587 | 760 | GAAAAATCCGTGGAACTGAAGCTGA | Antisense | 1960 |
| 215666_at | 830 | 167 | 774 | ACTGAAGCTGAGATCTTTAGTACGT | Antisense | 1961 |
| 215666_at | 643 | 643 | 783 | GAGATCTTTAGTACGTGGAGTCACC | Antisense | 1962 |
| 215666_at | 618 | 733 | 793 | GTACGTGGAGTCACCTTACAGATAC | Antisense | 1963 |
| 215666_at | 1159 | 845 | 799 | GGAGTCACCTTACAGATACAGAGCA | Antisense | 1964 |
| 215666_at | 560 | 1025 | 803 | TCACCTTACAGATACAGAGCATTTA | Antisense | 1965 |
| 215666_at | 373 | 631 | 819 | GAGCATTTATGCGGTATTCATTGGT | Antisense | 1966 |
| 215666_at | 288 | 1079 | 826 | TATGCGGTATTCATTGGTGCCTAAA | Antisense | 1967 |
| 215666_at | 878 | 717 | 832 | GTATTCATTGGTGCCTAAAGAACTT | Antisense | 1968 |
| 215666_at | 1006 | 209 | 848 | AAAGAACTTTAGGCATCCTCTGGAA | Antisense | 1969 |
| 215666_at | 66 | 1117 | 856 | TTAGGCATCCTCTGGAAAACCGGCC | Antisense | 1970 |
| 216438_s_at | 1086 | 185 | 150 | ACAGAGACGCAAGAGAAAAATCCAC | Antisense | 1971 |
| 216438_s_at | 546 | 277 | 168 | AATCCACTGCCTTCCAAAGAAACGA | Antisense | 1972 |
| 216438_s_at | 138 | 315 | 172 | CACTGCCTTCCAAAGAAACGATTGA | Antisense | 1973 |
| 216438_s_at | 670 | 849 | 200 | GGAGAAGCAAGCAGGCGAATCGTAA | Antisense | 1974 |
| 216438_s_at | 49 | 135 | 209 | AGCAGGCGAATCGTAATGAGGCGTG | Antisense | 1975 |
| 216438_s_at | 556 | 957 | 245 | TGCACTGTACATTCCACAAGCATTG | Antisense | 1976 |
| 216438_s_at | 569 | 167 | 248 | ACTGTACATTCCACAAGCATTGCCT | Antisense | 1977 |
| 216438_s_at | 9 | 109 | 308 | AGATGCAAAGAGGTTGGATCAAGTT | Antisense | 1978 |
| 216438_s_at | 178 | 259 | 328 | AAGTTTAAATGACTGTGCTGCCCCT | Antisense | 1979 |
| 216438_s_at | 744 | 591 | 426 | GAAAGAACTTGCATGTTGGTGAAGG | Antisense | 1980 |
| 216438_s_at | 432 | 499 | 516 | GCTGTAATGCAGTTTAATCAGAGTG | Antisense | 1981 |
| 216540_at | 167 | 635 | 37 | GAGAAGAAGTCCCCAACATGACTGT | Antisense | 1982 |
| 216540_at | 206 | 529 | 134 | GCAGTGGGTGTCCAGAGCAAACAAA | Antisense | 1983 |
| 216540_at | 837 | 251 | 159 | AAGAACTTATTTTGGCTGGACAGCA | Antisense | 1984 |
| 216540_at | 966 | 855 | 176 | GGACAGCAAGCAGAGTGCCAGTTCC | Antisense | 1985 |
| 216540_at | 201 | 113 | 187 | AGAGTGCCAGTTCCAAAGATGAGCT | Antisense | 1986 |
| 216540_at | 644 | 671 | 204 | GATGAGCTTGTTTGTAGCACCACCA | Antisense | 1987 |
| 216540_at | 479 | 741 | 246 | GTAATACATGGCTGAGTCTTCCTCT | Antisense | 1988 |
| 216540_at | 1134 | 499 | 256 | GCTGAGTCTTCCTCTTGAGCAAATG | Antisense | 1989 |
| 216540_at | 85 | 633 | 272 | GAGCAAATGGTTTCTTCAGGTGGAA | Antisense | 1990 |
| 216540_at | 969 | 815 | 290 | GGTGGAAAGATGTCTCGCCTTTGTT | Antisense | 1991 |
| 216540_at | 1120 | 751 | 301 | GTCTCGCCTTTGTTAAGGTCAGCAG | Antisense | 1992 |
| 216958_s_at | 460 | 925 | 2111 | TGTAATCCCAGTTACACAGAAGACT | Antisense | 1993 |
| 216958_s_at | 471 | 661 | 2294 | GAGGACAGCGTGCTTTTGTGTACTG | Antisense | 1994 |
| 216958_s_at | 1037 | 139 | 2300 | AGCGTGCTTTTGTGTACTGTTGGAA | Antisense | 1995 |
| 216958_s_at | 614 | 465 | 2408 | CCACAGCTGACTGGGCAGCAGGCAC | Antisense | 1996 |
| 216958_s_at | 34 | 947 | 2415 | TGACTGGGCAGCAGGCACAGGCCCT | Antisense | 1997 |
| 216958_s_at | 424 | 159 | 2440 | ACCCGAGCAGGCCGGAGTTGGCTCG | Antisense | 1998 |
| 216958_s_at | 533 | 415 | 2443 | CGAGCAGGCCGGAGTTGGCTCGCAT | Antisense | 1999 |
| 216958_s_at | 816 | 391 | 2482 | CTGCCTGTGTACATTTCTCCAGATA | Antisense | 2000 |
| 216958_s_at | 306 | 179 | 2492 | ACATTTCTCCAGATACCCTATGGCT | Antisense | 2001 |
| 216958_s_at | 1090 | 457 | 2500 | CCAGATACCCTATGGCTAATTTTGT | Antisense | 2002 |
| 216958_s_at | 57 | 881 | 2513 | GGCTAATTTTGTTATAACTGCACAG | Antisense | 2003 |
| 217147_s_at | 440 | 1003 | 1036 | TCTCCTTTCTCACCAATGGGCAATA | Antisense | 2004 |
| 217147_s_at | 398 | 981 | 1038 | TCCTTTCTCACCAATGGGCAATAGC | Antisense | 2005 |
| 217147_s_at | 256 | 1087 | 1149 | TATTTTTCCCAAAGATAGCTTTACT | Antisense | 2006 |
| 217147_s_at | 565 | 743 | 1186 | GTCAAATTAATGCATGCTCCTTACA | Antisense | 2007 |
| 217147_s_at | 863 | 275 | 1194 | AATGCATGCTCCTTACAACAAACAA | Antisense | 2008 |
| 217147_s_at | 1061 | 53 | 1199 | ATGCTCCTTACAACAAACAAATATC | Antisense | 2009 |
| 217147_s_at | 559 | 647 | 1229 | GAGTTTAGGAATTCTACTAGCCAGA | Antisense | 2010 |
| 217147_s_at | 368 | 173 | 1244 | ACTAGCCAGAGATAGTCACTTGGAG | Antisense | 2011 |
| 217147_s_at | 635 | 685 | 1254 | GATAGTCACTTGGAGAAACTTTCTA | Antisense | 2012 |
| 217147_s_at | 69 | 589 | 1268 | GAAACTTTCTATATATCCTTCTAAA | Antisense | 2013 |
| 217147_s_at | 102 | 319 | 1480 | CACCCCACTGATAACCAAATCACAG | Antisense | 2014 |
| 217778_at | 547 | 1073 | 1551 | TAGGGACTGGCGCCAAATGGTCTCT | Antisense | 2015 |
| 217778_at | 520 | 467 | 1581 | CCAATTTTGGTATCTTCTCTGGCCT | Antisense | 2016 |
| 217778_at | 32 | 381 | 1657 | CTCTTTCTCAAAGCACAGTAATGTG | Antisense | 2017 |
| 217778_at | 915 | 131 | 1698 | AGCACCTCAGTGAAGGGGGCCTGCT | Antisense | 2018 |
| 217778_at | 98 | 811 | 1778 | GGGGTAGGAGGAAGGTTGTTAGCAT | Antisense | 2019 |
| 217778_at | 246 | 1155 | 1793 | TTGTTAGCATCTACTGCTGCCGTAC | Antisense | 2020 |
| 217778_at | 567 | 393 | 1806 | CTGCTGCCGTACCCTAGGAATATGG | Antisense | 2021 |
| 217778_at | 631 | 605 | 1832 | GACATGGACATGGTGTCCCATGCCC | Antisense | 2022 |
| 217778_at | 1158 | 779 | 1844 | GTGTCCCATGCCCAGATGATAAACA | Antisense | 2023 |
| 217778_at | 545 | 59 | 1859 | ATGATAAACACTGAGCTGCCAAAAC | Antisense | 2024 |
| 217778_at | 750 | 191 | 1866 | ACACTGAGCTGCCAAAACATTTTTT | Antisense | 2025 |
| 218553_s_at | 1081 | 95 | 2857 | AGTCCTGTCTGTGTGGACTGGCACC | Antisense | 2026 |
| 218553_s_at | 804 | 533 | 2928 | GCACGTTACCTCAGGAATGGGCCCC | Antisense | 2027 |
| 218553_s_at | 376 | 345 | 2955 | CATGAAGGGGCCCATCTGTCAGCAG | Antisense | 2028 |
| 218553_s_at | 560 | 929 | 3059 | TGTTACACTAGCCTGCCGATGAGAC | Antisense | 2029 |
| 218553_s_at | 942 | 483 | 3073 | GCCGATGAGACCCAGACACAGGCAG | Antisense | 2030 |
| 218553_s_at | 971 | 357 | 3108 | CTTGACCCCTGATTCCAGTGAGGAC | Antisense | 2031 |
| 218553_s_at | 441 | 317 | 3170 | CACGACAGGCCCAAAGATGGACCCC | Antisense | 2032 |
| 218553_s_at | 218 | 987 | 3213 | TCCCCAAGTGTTCTCCGGTGGAGAA | Antisense | 2033 |
| 218553_s_at | 756 | 37 | 3272 | ATCACCATCCTGTGTACAATGGCTG | Antisense | 2034 |
| 218553_s_at | 1040 | 729 | 3296 | GTAGACTTGTATATGGCTCCTTTAA | Antisense | 2035 |
| 218553_s_at | 681 | 781 | 3358 | GTGTGAACACATTGCGTTCCCAGTC | Antisense | 2036 |
| 218723_s_at | 997 | 167 | 380 | ACTGAATTCTCCAACAGACTCTACC | Antisense | 2037 |
| 218723_s_at | 612 | 463 | 421 | CCACTGTCACTCCTCAGAAAGCTAA | Antisense | 2038 |
| 218723_s_at | 673 | 573 | 468 | GAAGCCTTCATTGCTGATCTTGACA | Antisense | 2039 |
| 218723_s_at | 644 | 997 | 523 | TCTGGGTCCTTTCATCATAAGGGAG | Antisense | 2040 |
| 218723_s_at | 837 | 473 | 550 | GCTTCAGAAAGTTCCGAGGACCTGC | Antisense | 2041 |
| 218723_s_at | 200 | 661 | 565 | GAGGACCTGCTAAAATCAGCTACTA | Antisense | 2042 |
| 218723_s_at | 641 | 1023 | 580 | TCAGCTACTAGAATCTGCTGCCAGA | Antisense | 2043 |
| 218723_s_at | 472 | 859 | 607 | GGACAAAGACGTGCACTCAACCTTC | Antisense | 2044 |
| 218723_s_at | 757 | 339 | 646 | CAGGCTCACCTTAAAATCAGCCCTT | Antisense | 2045 |
| 218723_s_at | 817 | 581 | 734 | GAACAGACGATCCATGCTAATATTG | Antisense | 2046 |
| 218723_s_at | 281 | 1065 | 841 | TAGCAGTAAGCTTTCCCATTATAAT | Antisense | 2047 |
| 218786_at | 423 | 967 | 6459 | TGCTGGCTGGCCATTTACTTCCAGC | Antisense | 2048 |
| 218786_at | 381 | 459 | 6479 | CCAGCCCTTATGAGGAGTTTCCCCT | Antisense | 2049 |
| 218786_at | 805 | 449 | 6553 | CCTTACCGGCTCCATATGGGGTACA | Antisense | 2050 |
| 218786_at | 828 | 1023 | 6592 | TCACCCCAACTTGGGAAACCCTCTG | Antisense | 2051 |
| 218786_at | 1024 | 389 | 6629 | CTCCAGAGCCCCTTGTGGGGTCAGT | Antisense | 2052 |
| 218786_at | 834 | 335 | 6650 | CAGTGAGACCTCATTGTGGCCACAT | Antisense | 2053 |
| 218786_at | 497 | 3 | 6662 | ATTGTGGCCACATTACAGCCCAGTG | Antisense | 2054 |
| 218786_at | 252 | 319 | 6763 | CACCTCAGTATCTGCTTTCGGGGAA | Antisense | 2055 |
| 218786_at | 38 | 1081 | 6771 | TATCTGCTTTCGGGGAACCCAACCT | Antisense | 2056 |
| 218786_at | 599 | 335 | 6819 | CAGTCCTGCAGGTTTGAACTCTGAC | Antisense | 2057 |
| 218786_at | 1007 | 609 | 6850 | GACTTTTCCAGTTATCTCGTGGAAT | Antisense | 2058 |
| 218838_s_at | 1101 | 795 | 1858 | GGGCATGGTAATTAGCCTTTCCCCA | Antisense | 2059 |
| 218838_s_at | 49 | 295 | 1904 | CAAGGGTCAACTGAATTCCCCACTT | Antisense | 2060 |
| 218838_s_at | 60 | 935 | 1944 | TGATCTCCTTTTAATTTCACGTCTA | Antisense | 2061 |
| 218838_s_at | 90 | 1127 | 1959 | TTCACGTCTAAGATCCTGGCAGCTT | Antisense | 2062 |
| 218838_s_at | 668 | 983 | 2006 | TCCTGCTGGGACTGTCAGCTCATTT | Antisense | 2063 |
| 218838_s_at | 1043 | 933 | 2108 | TGATTATTGCTGCCCTGTGGGTCTT | Antisense | 2064 |
| 218838_s_at | 313 | 749 | 2128 | GTCTTGCTCAATACTGTTCATACCT | Antisense | 2065 |
| 218838_s_at | 83 | 1075 | 2223 | TAGGCCCCTTAAATACTGTGTAGCA | Antisense | 2066 |
| 218838_s_at | 716 | 351 | 2261 | CATTACCAGCTGGGTCACCTTGGAT | Antisense | 2067 |
| 218838_s_at | 191 | 745 | 2274 | GTCACCTTGGATGGGTCTGTCAACA | Antisense | 2068 |
| 218838_s_at | 1033 | 653 | 2330 | GAGGGTAGTCCCTACCTCATAAGGG | Antisense | 2069 |
| 218956_s_at | 1109 | 1137 | 2115 | TTCCATAGGCCGATGCTCTGAAAGA | Antisense | 2070 |
| 218956_s_at | 508 | 637 | 2141 | GAGACGTGGGGCTCGAGAGATTTAA | Antisense | 2071 |
| 218956_s_at | 399 | 409 | 2374 | CGGCCCTGCAACGGAGGAGGACAGT | Antisense | 2072 |
| 218956_s_at | 483 | 659 | 2387 | GAGGAGGACAGTGAGACGATGCCAC | Antisense | 2073 |
| 218956_s_at | 106 | 433 | 2425 | CCCCTGCACACTCACATATGTGGCA | Antisense | 2074 |
| 218956_s_at | 218 | 391 | 2467 | CTGCCACCATGCCATATAGGGACAC | Antisense | 2075 |
| 218956_s_at | 1031 | 1055 | 2483 | TAGGGACACACCTCAGAAACCCTTC | Antisense | 2076 |
| 218956_s_at | 1015 | 451 | 2503 | CCTTCCTTGACAGCTCTGGACAGGG | Antisense | 2077 |
| 218956_s_at | 242 | 583 | 2527 | GAAAATTTGGCTCCCTCATGAAGGT | Antisense | 2078 |
| 218956_s_at | 728 | 503 | 2559 | GCTGCTGTTGACACCGAGGTTACAT | Antisense | 2079 |
| 218956_s_at | 570 | 61 | 2603 | ATGTTCTGCAAATCCAACACACGTT | Antisense | 2080 |
| 219343_at | 141 | 605 | 957 | GACAGTTCAGAATCATGTTCCCCAT | Antisense | 2081 |
| 219343_at | 842 | 987 | 975 | TCCCCATTCTGGTGTTGGATCTATA | Antisense | 2082 |
| 219343_at | 61 | 553 | 1009 | GAATCCTTACCACAGAGTCCAGATT | Antisense | 2083 |
| 219343_at | 807 | 333 | 1039 | CAGTATTCTATCAGTACAGCTCTCT | Antisense | 2084 |
| 219343_at | 201 | 507 | 1057 | GCTCTCTGCAGCTTAAACTCGGTGG | Antisense | 2085 |
| 219343_at | 869 | 253 | 1133 | AAGACTGCTGAGGCCAAGTGCTATT | Antisense | 2086 |
| 219343_at | 561 | 233 | 1176 | AACTTGGCTATTTTCTTGACACTTT | Antisense | 2087 |
| 219343_at | 789 | 1037 | 1277 | TAATGTGCTGCTAGGTTTTTTGTTT | Antisense | 2088 |
| 219343_at | 916 | 19 | 1373 | ATTTGCCTTTAATCTTATTGTTCTC | Antisense | 2089 |
| 219343_at | 765 | 577 | 1445 | GAACTGGTTGATTCTGGGAGGCATC | Antisense | 2090 |
| 219343_at | 464 | 905 | 1490 | TGGATATGTACATGTCCTATTCTAC | Antisense | 2091 |
| 219368_at | 427 | 93 | 1968 | AGTGTTGTGTAGCTTAATCCTTCTG | Antisense | 2092 |
| 219368_at | 214 | 927 | 1975 | TGTAGCTTAATCCTTCTGAAGTCTT | Antisense | 2093 |
| 219368_at | 1020 | 923 | 2002 | TGTCATGTAGCTATTAATCTGTGGC | Antisense | 2094 |
| 219368_at | 184 | 1103 | 2160 | TTTAATTTTGTTGTGCCTGCTTCAT | Antisense | 2095 |
| 219368_at | 195 | 763 | 2172 | GTGCCTGCTTCATTTTGCAATAACA | Antisense | 2096 |
| 219368_at | 1027 | 549 | 2247 | GAATTTCAGGCAAAACTACATTTAT | Antisense | 2097 |
| 219368_at | 17 | 1045 | 2278 | TAACAGCTTGTTCATAGGCTCTTGT | Antisense | 2098 |
| 219368_at | 466 | 1 | 2350 | ATTGTTTGTCGGTGAAGTGTTAGTC | Antisense | 2099 |
| 219368_at | 599 | 303 | 2385 | CAAAAGTTTGCACATATTGTTCTGT | Antisense | 2100 |
| 219368_at | 1054 | 1005 | 2443 | TCTCTTTTCCTATTACATCATTCAT | Antisense | 2101 |
| 219368_at | 1061 | 261 | 2471 | AAGTGATCACTTTACCATTTTGAAA | Antisense | 2102 |
| 219551_at | 696 | 579 | 285 | GAACAGGTGACCATAACTCTGCCAA | Antisense | 2103 |
| 219551_at | 242 | 67 | 370 | ATGTGGAATTCAGCCAGGACTCCCA | Antisense | 2104 |
| 219551_at | 56 | 473 | 382 | GCCAGGACTCCCAATCTTGTAAAAC | Antisense | 2105 |
| 219551_at | 709 | 107 | 420 | AGATAAGATGTCCCCAGCATCTCCA | Antisense | 2106 |
| 219551_at | 925 | 103 | 511 | AGTTCATCAGATTCCAAAAGTTCAT | Antisense | 2107 |
| 219551_at | 263 | 259 | 528 | AAGTTCATCATCTTCAAGTAGTGAG | Antisense | 2108 |
| 219551_at | 877 | 693 | 580 | GATTGCAAATCCTCTACTTCTGATA | Antisense | 2109 |
| 219551_at | 470 | 547 | 609 | GAATTGTGTCTCAGGACATCCTACC | Antisense | 2110 |
| 219551_at | 976 | 605 | 623 | GACATCCTACCATGACACAGTACAG | Antisense | 2111 |
| 219551_at | 529 | 107 | 676 | AGATTTCGAGACAACAGTGGCCTTC | Antisense | 2112 |
| 219551_at | 542 | 185 | 689 | ACAGTGGCCTTCTGATGAATACTTT | Antisense | 2113 |
| 219844_at | 717 | 39 | 597 | ATCAGGCACACTTTCTTCAGAGGCA | Antisense | 2114 |
| 219844_at | 399 | 729 | 647 | GTAGACGGGGTGGCATCATGGCATC | Antisense | 2115 |
| 219844_at | 640 | 897 | 665 | TGGCATCTTTATATACATCCCATCC | Antisense | 2116 |
| 219844_at | 510 | 1053 | 678 | TACATCCCATCCAGCTGACAATGGA | Antisense | 2117 |
| 219844_at | 444 | 1053 | 740 | TACAGGCTGTTTTGGAGGATACGTT | Antisense | 2118 |
| 219844_at | 665 | 585 | 861 | GAAAACCTAAAACAAGCCTCTTGCT | Antisense | 2119 |
| 219844_at | 849 | 183 | 907 | ACAGGAGTAGGTGCCACTGACCTCT | Antisense | 2120 |
| 219844_at | 73 | 169 | 922 | ACTGACCTCTATTGTTGGAGACTTT | Antisense | 2121 |
| 219844_at | 579 | 975 | 949 | TCCACTTTTTGTTTCAGCCAGTAAA | Antisense | 2122 |
| 219844_at | 1028 | 1089 | 976 | TATTGTTTTGCTTCATCTGTACACA | Antisense | 2123 |
| 219844_at | 367 | 603 | 1091 | GACAGCCTGTCATTGAATCTAAACA | Antisense | 2124 |
| 219901_at | 180 | 133 | 1257 | AGCAGAGGATGCTCATTCGGCTCAG | Antisense | 2125 |
| 219901_at | 656 | 673 | 1264 | GATGCTCATTCGGCTCAGAAGTGGA | Antisense | 2126 |
| 219901_at | 566 | 571 | 1291 | GAAGCATTTCAGGAAGGCACAATAT | Antisense | 2127 |
| 219901_at | 134 | 731 | 1317 | GTAGCAGTATTGGTTTCATCTCTTC | Antisense | 2128 |
| 219901_at | 930 | 715 | 1329 | GTTTCATCTCTTCTGTGATTCCAAA | Antisense | 2129 |
| 219901_at | 847 | 467 | 1415 | GCCAAGATAAAGCCAACCAGACCCT | Antisense | 2130 |
| 219901_at | 446 | 467 | 1427 | CCAACCAGACCCTTCATCAAAGAAA | Antisense | 2131 |
| 219901_at | 1071 | 1085 | 1545 | TATTTCTGAGACACACTGAATTCTA | Antisense | 2132 |
| 219901_at | 65 | 723 | 1612 | GTATACTGTATTAACTATTCGTGAC | Antisense | 2133 |
| 219901_at | 515 | 607 | 1634 | GACATAGTTCACACTGTTTTCTTAC | Antisense | 2134 |
| 219901_at | 23 | 1097 | 1725 | TTTCCACAAAGTGCTTGGTTTAGGA | Antisense | 2135 |
| 220005_at | 868 | 1131 | 2287 | TTCTTGCATCCTTCTGTGATTCAAA | Antisense | 2136 |
| 220005_at | 802 | 925 | 2392 | TGTAAATGTGCCACGAGCTCCAACA | Antisense | 2137 |
| 220005_at | 394 | 139 | 2407 | AGCTCCAACACGACCATCGTAGGGT | Antisense | 2138 |
| 220005_at | 739 | 965 | 2423 | TCGTAGGGTGAAGCCCACGTTTTCT | Antisense | 2139 |
| 220005_at | 1152 | 347 | 2456 | CTCAAAGGCCCTAGAACTTGCCTAC | Antisense | 2140 |
| 220005_at | 416 | 51 | 2508 | ATCCATCTCTTGAACTTTATACTCT | Antisense | 2141 |
| 220005_at | 993 | 589 | 2631 | GAAACCCTATGTTGTCATCACTTGG | Antisense | 2142 |
| 220005_at | 817 | 199 | 2662 | AAATATTACCTGGCCAATATCCCAC | Antisense | 2143 |
| 220005_at | 997 | 383 | 2714 | CTCTGTATGCTGTCACACCTATATT | Antisense | 2144 |
| 220005_at | 592 | 355 | 2757 | CATTGTAATTACTTCCTGACCTTTG | Antisense | 2145 |
| 220005_at | 837 | 943 | 2773 | TGACCTTTGTATCTACTCTTTTAGT | Antisense | 2146 |
| 220168_at | 1103 | 285 | 1229 | AATATCTTTCCTACTAGACACTCTC | Antisense | 2147 |
| 220168_at | 19 | 249 | 1299 | AAGCTTATCGACAGATGGCCCTACT | Antisense | 2148 |
| 220168_at | 373 | 487 | 1316 | GCCCTACTAAGTTCTGCTTTTGCAT | Antisense | 2149 |
| 220168_at | 327 | 849 | 1347 | GGAGCAAGTGGAACCTACTATGTAA | Antisense | 2150 |
| 220168_at | 1030 | 941 | 1423 | TGAGAATCCTAATTGGGCCCTTTTA | Antisense | 2151 |
| 220168_at | 714 | 773 | 1494 | GTGAGGCATTTTCTGAAGCACTTAA | Antisense | 2152 |
| 220168_at | 75 | 717 | 1531 | GTTTCATTCTACTTTATATCACATG | Antisense | 2153 |
| 220168_at | 503 | 593 | 1588 | GAAAGTCAGGAGTTCCAACTGTCAG | Antisense | 2154 |
| 220168_at | 749 | 301 | 1603 | CAACTGTCAGTTTGTCAACTCTGTG | Antisense | 2155 |
| 220168_at | 211 | 373 | 1644 | CTACCAGATTGCTCAGCTACTCCTA | Antisense | 2156 |
| 220168_at | 550 | 185 | 1721 | ACAGGTATTTCTGCAACACAATGTA | Antisense | 2157 |
| 220241_at | 566 | 419 | 1596 | CGCCAGTACATCTTCTAGCATTGTC | Antisense | 2158 |
| 220241_at | 992 | 333 | 1599 | CAGTACATCTTCTAGCATTGTCGTG | Antisense | 2159 |
| 220241_at | 732 | 1133 | 1628 | TTCTCCGAATCCTGGTTTTGATTGG | Antisense | 2160 |
| 220241_at | 449 | 553 | 1634 | GAATCCTGGTTTTGATTGGTCAGAT | Antisense | 2161 |
| 220241_at | 1112 | 905 | 1640 | TGGTTTTGATTGGTCAGATTCTTTT | Antisense | 2162 |
| 220241_at | 403 | 1111 | 1804 | TTAACGGTAATTCTCAAACTATGTG | Antisense | 2163 |
| 220241_at | 1087 | 825 | 1809 | GGTAATTCTCAAACTATGTGTTATT | Antisense | 2164 |
| 220241_at | 750 | 35 | 1849 | ATAATCTCTTTCACTGAAATCTTGT | Antisense | 2165 |
| 220241_at | 65 | 315 | 1860 | CACTGAAATCTTGTGATCCATTCTT | Antisense | 2166 |
| 220241_at | 204 | 41 | 1867 | ATCTTGTGATCCATTCTTTACCTTT | Antisense | 2167 |
| 220241_at | 576 | 1011 | 1881 | TCTTTACCTTTCCATTTTAATAATG | Antisense | 2168 |
| 220301_at | 373 | 35 | 1467 | ATAAGTCATTTCTAATCTTTGTATA | Antisense | 2169 |
| 220301_at | 603 | 605 | 1572 | GACAGGCTGTAAGCATCGCTGAGAA | Antisense | 2170 |
| 220301_at | 1151 | 77 | 1603 | AGGACTTTTGACTTTTATCTGGATA | Antisense | 2171 |
| 220301_at | 333 | 39 | 1645 | ATCATGGAAAGGCATCAGCATTGCA | Antisense | 2172 |
| 220301_at | 931 | 519 | 1675 | GCATCTAGGTAGAAATCAGGCCAAA | Antisense | 2173 |
| 220301_at | 70 | 303 | 1696 | CAAAATTAAGCTGTGGTTTCCCTCT | Antisense | 2174 |
| 220301_at | 417 | 829 | 1710 | GGTTTCCCTCTGAGTAGTGGGAATA | Antisense | 2175 |
| 220301_at | 313 | 253 | 1851 | AAGACACTTATAATTTTCCATACCT | Antisense | 2176 |
| 220301_at | 975 | 15 | 1863 | ATTTTCCATACCTATTTTCAACTGA | Antisense | 2177 |
| 220301_at | 1035 | 543 | 1890 | GCAACTTGTAAGATTTAACTCAGTC | Antisense | 2178 |
| 220301_at | 759 | 293 | 1914 | CAATAACATACTGGTTTTACTCATC | Antisense | 2179 |
| 220933_s_at | 134 | 699 | 1751 | GTTGTTTACTGAATCCTGGTGTGAA | Antisense | 2180 |
| 220933_s_at | 1142 | 371 | 1800 | CTACTGCTGTACTTCCGATTTACGG | Antisense | 2181 |
| 220933_s_at | 6 | 649 | 1867 | GAGTACCTATTTATGTGGGCCTTCA | Antisense | 2182 |
| 220933_s_at | 677 | 783 | 1881 | GTGGGCCTTCAGTGGATGGGCAGTG | Antisense | 2183 |
| 220933_s_at | 169 | 197 | 1990 | ACAAGTAGTCCTGTTTGCCACTAAA | Antisense | 2184 |
| 220933_s_at | 132 | 711 | 2114 | GTTATCTCTGCCAGTCACAAGTGTG | Antisense | 2185 |
| 220933_s_at | 35 | 197 | 2130 | ACAAGTGTGGCTGGTGTCATTCTGG | Antisense | 2186 |
| 220933_s_at | 498 | 1015 | 2146 | TCATTCTGGGTCTGACTGGAGCCCT | Antisense | 2187 |
| 220933_s_at | 496 | 387 | 2169 | CTCCTGGACTGTTTCTTTAATTTCA | Antisense | 2188 |
| 220933_s_at | 166 | 145 | 2196 | AGCCCTGCAGACATAGTACCTGGTC | Antisense | 2189 |
| 220933_s_at | 315 | 1061 | 2212 | TACCTGGTCAGAACTATGCCTCGGT | Antisense | 2190 |
| 221205_at | 764 | 211 | 1553 | AAAGCTTATCCACCACGATTAAGCC | Antisense | 2191 |
| 221205_at | 519 | 321 | 1563 | CACCACGATTAAGCCGGCTTCATCC | Antisense | 2192 |
| 221205_at | 1007 | 545 | 1595 | GCAAGGCTGGTTCAGCATACACAAA | Antisense | 2193 |
| 221205_at | 349 | 223 | 1659 | AAAAGCCACATGATATTTACCTGTA | Antisense | 2194 |
| 221205_at | 152 | 935 | 1669 | TGATATTTACCTGTATACCTTTTTA | Antisense | 2195 |
| 221205_at | 355 | 499 | 1711 | GCTGTCATTATTTATGCTAACACTG | Antisense | 2196 |
| 221205_at | 211 | 509 | 1726 | GCTAACACTGGTTTTGTGTCCCTGG | Antisense | 2197 |
| 221205_at | 72 | 781 | 1741 | GTGTCCCTGGAATCTATCTATCTAA | Antisense | 2198 |
| 221205_at | 436 | 1111 | 1786 | TTTTCCATTTTCCAGTACCTATTAG | Antisense | 2199 |
| 221205_at | 638 | 121 | 1809 | AGACAGAATGGCTTTCAATTTTTTC | Antisense | 2200 |
| 221205_at | 310 | 1099 | 1830 | TTTCTCTTACTTCCAAACTTAGTCA | Antisense | 2201 |
| 221728_x_at | 1005 | 15 | 7681 | ATTTTCTAGTTTTCATTCTGTACAT | Antisense | 2202 |
| 221728_x_at | 576 | 449 | 7743 | CCTTTCAATGTGTGGTATCAGCTGG | Antisense | 2203 |
| 221728_x_at | 329 | 1081 | 7758 | TATCAGCTGGACTCAGTAACACCCC | Antisense | 2204 |
| 221728_x_at | 40 | 1101 | 7784 | TTTCTTCAGCTGGGGATGGGGAATG | Antisense | 2205 |
| 221728_x_at | 1028 | 1043 | 7840 | TAAAAGCCTTCCTTTCACAGTTTCT | Antisense | 2206 |
| 221728_x_at | 232 | 1029 | 7854 | TCACAGTTTCTGGCATCACTACCAC | Antisense | 2207 |
| 221728_x_at | 485 | 1115 | 7908 | TTATCATCATCTGCTTTATTCACAT | Antisense | 2208 |
| 221728_x_at | 893 | 11 | 7979 | ATTAAGTGGCTTCGTCATTGTCCTT | Antisense | 2209 |
| 221728_x_at | 666 | 1015 | 7993 | TCATTGTCCTTCTACCTCAAAGATA | Antisense | 2210 |
| 221728_x_at | 151 | 45 | 8082 | ATCTCTTTTGAGTCTTTGCTGTTTG | Antisense | 2211 |
| 221728_x_at | 28 | 1155 | 8122 | TTGTTCAGCATGGGTGACCACCAGA | Antisense | 2212 |
| 221759_at | 41 | 323 | 917 | CAGCCTAGCCTTCAAGTGGTGTGAG | Antisense | 2213 |
| 221759_at | 257 | 817 | 934 | GGTGTGAGCGGCCTGAGTGGATACA | Antisense | 2214 |
| 221759_at | 670 | 91 | 949 | AGTGGATACACGTGGATAGCCGGCC | Antisense | 2215 |
| 221759_at | 534 | 971 | 977 | TGCCTCCCTGAGCCGTGACTCAGGG | Antisense | 2216 |
| 221759_at | 709 | 367 | 1040 | CTATGCCCAGGTGCGTCGGGCACAG | Antisense | 2217 |
| 221759_at | 179 | 127 | 1078 | AGAAGATAGCCTGCCTTGTGCTGGC | Antisense | 2218 |
| 221759_at | 673 | 1025 | 1150 | TCAGCCTCTTCTACATTTTCAATTT | Antisense | 2219 |
| 221759_at | 489 | 385 | 1212 | CTCGTGCCCTGGGCAGTGCACATGT | Antisense | 2220 |
| 221759_at | 945 | 875 | 1223 | GGCAGTGCACATGTTCAGTGCCCAG | Antisense | 2221 |
| 221759_at | 892 | 307 | 1306 | CACAAAGCCAACACTCTGTGACCAC | Antisense | 2222 |
| 221759_at | 443 | 485 | 1417 | GCCCCAAAGATGGGCCTTCTCTCTC | Antisense | 2223 |
| 221789_x_at | 563 | 1135 | 3835 | TTCTCACTCTACAGGGTCCTGGTGA | Antisense | 2224 |
| 221789_x_at | 1034 | 951 | 3857 | TGAAGAGCCAGTGAGGCCCCTGGTA | Antisense | 2225 |
| 221789_x_at | 346 | 959 | 3928 | TGCAGGGGCAGCACAGCTGGGGTGC | Antisense | 2226 |
| 221789_x_at | 595 | 1001 | 4016 | TCTGCAGCGGGGCCTTATGCTGCCA | Antisense | 2227 |
| 221789_x_at | 563 | 875 | 4082 | GGCAGGTGGCTGAGCAGGAGCTCCC | Antisense | 2228 |
| 221789_x_at | 220 | 319 | 4137 | CACCCCTGGGCATCATGTGTGTGGG | Antisense | 2229 |
| 221789_x_at | 171 | 339 | 4173 | CAGGTGTGGGAGCTGGTGACCCCAG | Antisense | 2230 |
| 221789_x_at | 80 | 111 | 4196 | AGACCCAGAATTCTCAGGGCTCTAC | Antisense | 2231 |
| 221789_x_at | 507 | 367 | 4225 | CTTTCCTGGTCCTAGGTGGCCAGTG | Antisense | 2232 |
| 221789_x_at | 886 | 483 | 4322 | GCCGTTTCCGTGGTTGTAGCAGAGG | Antisense | 2233 |
| 221789_x_at | 217 | 661 | 4343 | GAGGACCGGAGGTTGGGTTCCTGAT | Antisense | 2234 |
| 221874_at | 711 | 407 | 5130 | CTGGCCTACGTGTTCAATTTTCTAT | Antisense | 2235 |
| 221874_at | 349 | 305 | 5158 | CAAAGGCTTTAGTCCTTGACCCAGG | Antisense | 2236 |
| 221874_at | 1021 | 455 | 5178 | CCAGGGCTAAAGTGGTCTGTCCAAG | Antisense | 2237 |
| 221874_at | 557 | 263 | 5187 | AAGTGGTCTGTCCAAGCTGTTGTTG | Antisense | 2238 |
| 221874_at | 867 | 785 | 5330 | GTGGTGTATTCAAGCAGTAGGGTTT | Antisense | 2239 |
| 221874_at | 1042 | 1105 | 5391 | TTTTTGTCTGACTACATTAAAGATA | Antisense | 2240 |
| 221874_at | 1043 | 119 | 5416 | AGACTGACTATATTTATACAACAGA | Antisense | 2241 |
| 221874_at | 1037 | 1023 | 5460 | TCAGCTTTGTGAAATCGAATTTTTT | Antisense | 2242 |
| 221874_at | 887 | 979 | 5506 | TCCTTTTTACCCTGTAATCCAAGCG | Antisense | 2243 |
| 221874_at | 1052 | 1059 | 5513 | TACCCTGTAATCCAAGCGTTAATAG | Antisense | 2244 |
| 221874_at | 111 | 567 | 5545 | GAAGATGGGTTATTGCATGTCACTT | Antisense | 2245 |
| 221905_at | 3 | 997 | 4804 | TCTGGGTTTAGGGCTAGCCCTGCCT | Antisense | 2246 |
| 221905_at | 345 | 1009 | 4884 | TCTAGCTCTGACAGCCTAGAAGTCC | Antisense | 2247 |
| 221905_at | 699 | 809 | 4947 | GGGTGAATGAGCTTGTCAATGTGAT | Antisense | 2248 |
| 221905_at | 806 | 867 | 5005 | GGAATCTAAATGAAGCCAGCCCTCG | Antisense | 2249 |
| 221905_at | 39 | 1049 | 5011 | TAAATGAAGCCAGCCCTCGGTATCT | Antisense | 2250 |
| 221905_at | 991 | 995 | 5027 | TCGGTATCTGCAGGTTTCTCATCCA | Antisense | 2251 |
| 221905_at | 1107 | 665 | 5100 | GATGGTTACATCCGTATTGAACATG | Antisense | 2252 |
| 221905_at | 498 | 299 | 5162 | CAAGAACTCTTTATGTAGCATTTAC | Antisense | 2253 |
| 221905_at | 800 | 745 | 5248 | GTCAGTTATATGCAAATTCTGTACC | Antisense | 2254 |
| 221905_at | 428 | 723 | 5278 | GTATCAGGGAATTGAGCATCTTCAG | Antisense | 2255 |
| 221905_at | 495 | 943 | 5290 | TGAGCATCTTCAGATGTTGGTATCT | Antisense | 2256 |
| 222462_s_at | 472 | 1021 | 3171 | TCAGAGTGGTTTCATTGCCTTCCTA | Antisense | 2257 |
| 222462_s_at | 93 | 1037 | 3203 | TAATGGCCCCTCCATTTATTTGACT | Antisense | 2258 |
| 222462_s_at | 940 | 211 | 3228 | AAAGCATCACACAGTGGCACTAGCA | Antisense | 2259 |
| 222462_s_at | 623 | 283 | 3272 | AATACAGTGCTTTATGGCTCTAACA | Antisense | 2260 |
| 222462_s_at | 879 | 987 | 3418 | TCCCCGCTCCTAATGGTACGTGGGT | Antisense | 2261 |
| 222462_s_at | 923 | 1061 | 3486 | TACCTCCCTATCAGTTCTAGCATAG | Antisense | 2262 |
| 222462_s_at | 295 | 807 | 3544 | GGGTTTTCCTAGTATACCCACTGCA | Antisense | 2263 |
| 222462_s_at | 957 | 501 | 3592 | GCTGCTTCCAGGTATGGGACCTGCT | Antisense | 2264 |
| 222462_s_at | 263 | 801 | 3607 | GGGACCTGCTAAGTGTGGAATTACC | Antisense | 2265 |
| 222462_s_at | 193 | 195 | 3651 | ACAAGGAGGGCCTCTGGTGTTCCTG | Antisense | 2266 |
| 222462_s_at | 935 | 503 | 3686 | GCTGCCCACAAGCCATAAACCAATA | Antisense | 2267 |
| 222520_s_at | 959 | 231 | 2475 | AACATGCTCACATACACAGATCTTT | Antisense | 2268 |
| 222520_s_at | 28 | 1097 | 2503 | TTTCCTTACTAGACCTCTCTTTAGT | Antisense | 2269 |
| 222520_s_at | 589 | 823 | 2697 | TGGTCTTTGGGAGGAGTCTAGTACA | Antisense | 2270 |
| 222520_s_at | 101 | 551 | 2746 | GAATTTATTTTCATGGCTGTTGTCA | Antisense | 2271 |
| 222520_s_at | 1048 | 37 | 2825 | ATCAGAACTCTCCTTAAAATGCATT | Antisense | 2272 |
| 222520_s_at | 528 | 37 | 2853 | ATCAGAAGCAAAGTACACCCATTAT | Antisense | 2273 |
| 222520_s_at | 424 | 941 | 2915 | TGAGTAGTATAAGCCACCTATTTTT | Antisense | 2274 |
| 222520_s_at | 620 | 369 | 2932 | CTATTTTTACTTCCTTCTCTGAAAT | Antisense | 2275 |
| 222520_s_at | 962 | 5 | 2957 | ATTCCAGTTAACTCAGCTTGGGTTC | Antisense | 2276 |
| 222520_s_at | 101 | 389 | 2989 | CTCCTCTCTTCCTATAGAACACTGT | Antisense | 2277 |
| 222520_s_at | 26 | 125 | 3004 | AGAACACTGTGCATACCTTGAATAA | Antisense | 2278 |
| 222654_at | 521 | 637 | 1981 | GAGACACATGATGATTTGCATTGTA | Antisense | 2279 |
| 222654_at | 1082 | 207 | 2052 | AAAGTGTGTTTGTACCCATGAGTTC | Antisense | 2280 |
| 222654_at | 370 | 161 | 2065 | ACCCATGAGTTCAGCATTTCTGCCA | Antisense | 2281 |
| 222654_at | 256 | 673 | 2118 | GATGCATGGTTGCATCCTTTTCCTT | Antisense | 2282 |
| 222654_at | 239 | 1097 | 2136 | TTTCCTTCTGACCAACACTTAGTCT | Antisense | 2283 |
| 222654_at | 74 | 625 | 2145 | GACCAACACTTAGTCTTTAACTTTG | Antisense | 2284 |
| 222654_at | 613 | 265 | 2183 | AAGGTTTCAGGGTTAGCACTATAGT | Antisense | 2285 |
| 222654_at | 652 | 369 | 2201 | CTATAGTTTTGGTTCATTTTCTCAT | Antisense | 2286 |
| 222654_at | 402 | 955 | 2302 | TGAATTGAACTGCCTTTCATTATCT | Antisense | 2287 |
| 222654_at | 50 | 727 | 2403 | GTATGGTTGCTTTTGCCATGGTTGC | Antisense | 2288 |
| 222654_at | 822 | 455 | 2418 | CCATGGTTGCTTTTACTATTAATAA | Antisense | 2289 |
| 222778_s_at | 537 | 1157 | 5611 | TTGTGAACCATTTAACCCTTTTGAT | Antisense | 2290 |
| 222778_s_at | 117 | 155 | 5683 | ACCTCTGCATCAGTTGCTTCTATAG | Antisense | 2291 |
| 222778_s_at | 107 | 517 | 5698 | GCTTCTATAGAGCATCAGCCCTCAT | Antisense | 2292 |
| 222778_s_at | 224 | 3 | 5728 | ATTGTCTTAATGTCGGGGAGCAGCC | Antisense | 2293 |
| 222778_s_at | 925 | 423 | 5764 | CCCAGGTGTCCGCATTTTTGCATGG | Antisense | 2294 |
| 222778_s_at | 830 | 861 | 5792 | GGAAAAGGCTTACTTGCCCTTCAGC | Antisense | 2295 |
| 222778_s_at | 601 | 765 | 5834 | GTGCTATTCACAGTACCATGCGAGT | Antisense | 2296 |
| 222778_s_at | 861 | 407 | 5854 | CGAGTAGCCCACAGTTAATTCCCGG | Antisense | 2297 |
| 222778_s_at | 124 | 291 | 5870 | AATTCCCGGCAGATTCTTTGGCTGT | Antisense | 2298 |
| 222778_s_at | 1035 | 1039 | 5961 | TAAGATGCTTAGGGTGCCTGGCCAG | Antisense | 2299 |
| 222778_s_at | 862 | 387 | 5988 | CTCCGTGGTTTCCTGTTGTGACAGT | Antisense | 2300 |
| 222802_at | 248 | 59 | 1555 | ATGAGTCTACCTCACCTATATTGCA | Antisense | 2301 |
| 222802_at | 157 | 539 | 1577 | GCACTCTGGCAGAAGTATTTCCCAC | Antisense | 2302 |
| 222802_at | 863 | 311 | 1599 | CACATTTAATTATTGCCTCCCCAAA | Antisense | 2303 |
| 222802_at | 607 | 423 | 1657 | CCCATACTAAATCCTAGCCTCGTAG | Antisense | 2304 |
| 222802_at | 893 | 49 | 1667 | ATCCTAGCCTCGTAGAAGTCTGGTC | Antisense | 2305 |
| 222802_at | 587 | 261 | 1682 | AAGTCTGGTCTAATGTGTCAGCAGT | Antisense | 2306 |
| 222802_at | 598 | 825 | 1724 | GGTAATCTACTAGCTCTGATCCATA | Antisense | 2307 |
| 222802_at | 1012 | 39 | 1768 | ATCAGGAGATTCCCTGTCCTTGATT | Antisense | 2308 |
| 222802_at | 309 | 1097 | 1874 | TTTTCCTTTATATAACCGGCTAATG | Antisense | 2309 |
| 222802_at | 1113 | 913 | 1994 | TGGGCAGGCCATATTGGTCTATGTA | Antisense | 2310 |
| 222802_at | 858 | 181 | 2053 | ACATGCTTTGTTTTGCCTGTCAAGG | Antisense | 2311 |
| 222858_s_at | 88 | 415 | 969 | CGATCTCGGTCGTTCATCTTTAAAT | Antisense | 2312 |
| 222858_s_at | 47 | 469 | 1005 | GCCAAGGAATGCTCTGGCCCAGGAG | Antisense | 2313 |
| 222858_s_at | 88 | 619 | 1049 | GACGCTGTGATCTGCAGCAGGCTTC | Antisense | 2314 |
| 222858_s_at | 1127 | 453 | 1136 | CCATATACCACGTTGCTGACTCACG | Antisense | 2315 |
| 222858_s_at | 193 | 1123 | 1231 | TTAAACCACCACTCTTAGGTCTGCT | Antisense | 2316 |
| 222858_s_at | 793 | 1073 | 1246 | TAGGTCTGCTCACTCTTAGAACACA | Antisense | 2317 |
| 222858_s_at | 720 | 1155 | 1291 | TTGTTTTCACTCATTGTGGTCCCCA | Antisense | 2318 |
| 222858_s_at | 57 | 601 | 1324 | GACACTAGTTGCCTAGAGTCCCACT | Antisense | 2319 |
| 222858_s_at | 251 | 757 | 1341 | GTCCCACTGTGAGTCATGGTCAGCC | Antisense | 2320 |
| 222858_s_at | 292 | 907 | 1357 | TGGTCAGCCTGTCTGACATCCAGGT | Antisense | 2321 |
| 222858_s_at | 506 | 843 | 1414 | GGAGGCTTAGATGACTTCTGCAGGA | Antisense | 2322 |
| 222859_s_at | 695 | 1115 | 1837 | TTATCTTTATCCAACATTTCTCCAA | Antisense | 2323 |
| 222859_s_at | 824 | 777 | 1890 | GTGTATTATAACAATCCCTTTCCCA | Antisense | 2324 |
| 222859_s_at | 454 | 197 | 1900 | ACAATCCCTTTCCCAGAATTAGTTG | Antisense | 2325 |
| 222859_s_at | 995 | 547 | 1915 | GAATTAGTTGTATAGGGTTGGCCCA | Antisense | 2326 |
| 222859_s_at | 1135 | 123 | 1949 | AGAAAAATCTCGCATTGCTCCCTAA | Antisense | 2327 |
| 222859_s_at | 105 | 541 | 1996 | GCAACTTCTATTTCCAGTCGGGGGA | Antisense | 2328 |
| 222859_s_at | 812 | 773 | 2067 | GTGTTCCAAAGCTTTTTCAAACTAG | Antisense | 2329 |
| 222859_s_at | 125 | 645 | 2115 | GAGATACTGATGTCCACAGTTCATT | Antisense | 2330 |
| 222859_s_at | 959 | 703 | 2133 | GTTCATTGGCAGAATCTAACCCCTT | Antisense | 2331 |
| 222859_s_at | 747 | 507 | 2269 | GCTCTTGCAGTCCAAAGGGATACCT | Antisense | 2332 |
| 222859_s_at | 662 | 231 | 2316 | AACATCTCAGACGCAAAAATTACAT | Antisense | 2333 |
| 223095_at | 224 | 971 | 2789 | TGCCTGTAGCATTCCAGAGCTCACT | Antisense | 2334 |
| 223095_at | 330 | 377 | 2858 | CTCACTCTGTCTGCCAGGTATGAGA | Antisense | 2335 |
| 223095_at | 223 | 127 | 2880 | AGAAGAACACGTAAGACCGCCACCA | Antisense | 2336 |
| 223095_at | 975 | 391 | 2952 | CTGCCCCCAGAACTTTTGGATACTG | Antisense | 2337 |
| 223095_at | 448 | 843 | 2976 | GGAGGCAGTTGCATAGGTCTCCCTC | Antisense | 2338 |
| 223095_at | 239 | 489 | 3024 | GCCCAAGACTACTCTGGGCAGCTCC | Antisense | 2339 |
| 223095_at | 148 | 861 | 3079 | GGAAAGGATTTCTACAGTGTTCTAT | Antisense | 2340 |
| 223095_at | 417 | 115 | 3114 | AGAGAGAGTGGGTTTGGGAAGAGTG | Antisense | 2341 |
| 223095_at | 141 | 697 | 3145 | GTTGGGGAGAGGGGACCGATGTGCC | Antisense | 2342 |
| 223095_at | 136 | 621 | 3158 | GACCGATGTGCCTCATTGTTTAGTG | Antisense | 2343 |
| 223095_at | 581 | 1139 | 3201 | TTCTGGATAAAGTTTGGTTGTTTGC | Antisense | 2344 |
| 223198_x_at | 801 | 573 | 449 | GAAGCCTGACACCTTCAGGGACCAG | Antisense | 2345 |
| 223198_x_at | 666 | 623 | 468 | GACCAGCTCCAGGAGCTCTGCATCC | Antisense | 2346 |
| 223198_x_at | 23 | 1155 | 550 | TTGATTCTGTGGCCCAGCAGCAGGG | Antisense | 2347 |
| 223198_x_at | 801 | 391 | 582 | CTGCCGCATGTTGCTGACTTTCGGT | Antisense | 2348 |
| 223198_x_at | 998 | 627 | 659 | GAGCGTCCTGATGCAGCTGAAGCTT | Antisense | 2349 |
| 223198_x_at | 862 | 911 | 689 | TGGGTCAGCATACCGCTTTGAGGTC | Antisense | 2350 |
| 223198_x_at | 313 | 469 | 720 | GCCAAGTTCCAGGAGCTGCGGTACA | Antisense | 2351 |
| 223198_x_at | 1158 | 1027 | 817 | TCACTTGACCAGTCCCATTCAGATC | Antisense | 2352 |
| 223198_x_at | 1125 | 821 | 865 | GGTGCCAAAGTGCAGCTGACTCTTC | Antisense | 2353 |
| 223198_x_at | 639 | 1141 | 907 | TTCCCATGAGGCAGGCTCTTCAGTG | Antisense | 2354 |
| 223198_x_at | 985 | 629 | 977 | GAGCTATGCCTTTTTTTCTTTTTGG | Antisense | 2355 |
| 223284_at | 959 | 109 | 860 | AGAGGCCTGCCGAGGGGAGGAGCCT | Antisense | 2356 |
| 223284_at | 323 | 761 | 892 | GTCCACCCGTCAGCAGTGTGAAGTC | Antisense | 2357 |
| 223284_at | 530 | 261 | 912 | AAGTCTGTTGTGTTTGAGCTTCTCA | Antisense | 2358 |
| 223284_at | 624 | 375 | 933 | CTCAGAGTGGAATGACTCCTTTTCC | Antisense | 2359 |
| 223284_at | 503 | 429 | 1036 | CCCGGGTGATGTCTGCAAAGTCTGT | Antisense | 2360 |
| 223284_at | 334 | 923 | 1045 | TGTCTGCAAAGTCTGTGCTGTCCGT | Antisense | 2361 |
| 223284_at | 346 | 497 | 1077 | GCTGGGAGAGCTATCTGGGGAGGGG | Antisense | 2362 |
| 223284_at | 616 | 411 | 1112 | CGAGCAGAATACACCCCAGAGTTAG | Antisense | 2363 |
| 223284_at | 994 | 325 | 1128 | CAGAGTTAGGGTTTGCGACTCCGCC | Antisense | 2364 |
| 223284_at | 116 | 1153 | 1202 | TTGACTGCTTAGGAGGCGCGACGCA | Antisense | 2365 |
| 223284_at | 1161 | 403 | 1252 | CTGGGAAGGTTGAGAGCTGAGACGG | Antisense | 2366 |
| 223407_at | 512 | 933 | 1041 | TGTTGGAGCGCAGGGACCTGTGGCG | Antisense | 2367 |
| 223407_at | 533 | 243 | 1139 | AACCAGCGGCTGGAAACACTGACCA | Antisense | 2368 |
| 223407_at | 909 | 899 | 1149 | TGGAAACACTGACCAAGCTGCTCCA | Antisense | 2369 |
| 223407_at | 538 | 625 | 1180 | GAGCCAGCTGCTGCGTGAGCTGGTA | Antisense | 2370 |
| 223407_at | 387 | 393 | 1212 | CTGCTGGGGCAGACTCACTGAGAGC | Antisense | 2371 |
| 223407_at | 478 | 473 | 1286 | GCCATCAAGATCTTTTCTCGGCCCA | Antisense | 2372 |
| 223407_at | 35 | 1141 | 1316 | TTCGTGAAGATGGACGACTGAGCCC | Antisense | 2373 |
| 223407_at | 647 | 545 | 1356 | GCAAGGAGCTCCATGCTGAGGATCG | Antisense | 2374 |
| 223407_at | 1123 | 53 | 1368 | ATGCTGAGGATCGCCACATGGCTGC | Antisense | 2375 |
| 223407_at | 734 | 839 | 1375 | GGATCGCCACATGGCTGCAAAGGAC | Antisense | 2376 |
| 223407_at | 354 | 855 | 1406 | GGACCCAATCCCAGAGCAGAGGTCT | Antisense | 2377 |
| 223571_at | 616 | 305 | 999 | CAAAGTCATCTGAACTTCCGTTTCC | Antisense | 2378 |
| 223571_at | 849 | 1077 | 1124 | TATCAAGGTGCTAAGGCCCGGGTGG | Antisense | 2379 |
| 223571_at | 639 | 821 | 1182 | GGTGCTGCCTTTACAAACACCTGCA | Antisense | 2380 |
| 223571_at | 376 | 457 | 1227 | CCAGGCTTTAGAGCCCTCAGCAGGT | Antisense | 2381 |
| 223571_at | 168 | 427 | 1240 | CCCTCAGCAGGTCTGGGGAGCTAGA | Antisense | 2382 |
| 223571_at | 1133 | 303 | 1266 | CAAAGGAGGGACCTCAGGCCTTCCG | Antisense | 2383 |
| 223571_at | 713 | 451 | 1353 | CCTTCTCCCCAGAGGGAGGCGGCCT | Antisense | 2384 |
| 223571_at | 546 | 453 | 1382 | CCATTGGTGCTCATGCAGACTCTGG | Antisense | 2385 |
| 223571_at | 607 | 483 | 1445 | GCCGAGGGAGCCGTGGCTCCATGGC | Antisense | 2386 |
| 223571_at | 987 | 881 | 1459 | GGCTCCATGGCCAGATGACGGAAAC | Antisense | 2387 |
| 223571_at | 339 | 253 | 1505 | AAGACCTGTGCTATAAACCACCCTG | Antisense | 2388 |
| 223645_s_at | 600 | 1101 | 717 | TTTAGCAGTGCCTGTGACGCATTCC | Antisense | 2389 |
| 223645_s_at | 547 | 499 | 745 | GCTGACCTGGATTCTTCCAATATGC | Antisense | 2390 |
| 223645_s_at | 1002 | 189 | 773 | ACACTTCCTCTAAAAGAGCCCCAGG | Antisense | 2391 |
| 223645_s_at | 871 | 339 | 794 | CAGGAGTCCATCTGGAGGCTGACCC | Antisense | 2392 |
| 223645_s_at | 252 | 225 | 837 | AAAATGCTACTCAAAAGCCCTCTCC | Antisense | 2393 |
| 223645_s_at | 324 | 311 | 861 | CACAGGATCTCCTCTAGGCATTGAT | Antisense | 2394 |
| 223645_s_at | 130 | 125 | 1009 | AGAACTACTCAACTGTGTGGTAACA | Antisense | 2395 |
| 223645_s_at | 731 | 1061 | 1044 | TACCAATTTCCCCAACTATGTTGCA | Antisense | 2396 |
| 223645_s_at | 929 | 555 | 1096 | GAATGCATGTTTTCATTGCCTTCTC | Antisense | 2397 |
| 223645_s_at | 747 | 733 | 1131 | GTACGTTTGTGTGTTCATCACCTTA | Antisense | 2398 |
| 223645_s_at | 41 | 385 | 1220 | CTCTGGAGCCCATAATATCTACATA | Antisense | 2399 |
| 223646_s_at | 435 | 225 | 1999 | AAAATACTGGACCCACTTCTTTCAG | Antisense | 2400 |
| 223646_s_at | 476 | 565 | 2031 | GAAGATACCTTATATGCCCTAAAGT | Antisense | 2401 |
| 223646_s_at | 1055 | 485 | 2046 | GCCCTAAAGTTAATACCAGCAGTCA | Antisense | 2402 |
| 223646_s_at | 612 | 885 | 2131 | GGCTGTGACACTTCTTTGTGGTACT | Antisense | 2403 |
| 223646_s_at | 526 | 159 | 2363 | ACCCTTTGCAGAAGATTCCCTTGTA | Antisense | 2404 |
| 223646_s_at | 989 | 1139 | 2378 | TTCCCTTGTAAATGGCCCTGTGGCA | Antisense | 2405 |
| 223646_s_at | 534 | 877 | 2399 | GGCATGCCCAGTATCTGCAATGTTC | Antisense | 2406 |
| 223646_s_at | 253 | 871 | 2437 | GGCAAACCTCTCTGTTTGCCAAGCC | Antisense | 2407 |
| 223646_s_at | 175 | 717 | 2450 | GTTTGCCAAGCCTGCAGAGTTGAAC | Antisense | 2408 |
| 223646_s_at | 979 | 1049 | 2490 | TACAATCTGACCCTGTTTTTTGGCC | Antisense | 2409 |
| 223646_s_at | 176 | 429 | 2513 | CCCTGTTTCTGGACACTGTAGCTGA | Antisense | 2410 |
| 224588_at | 789 | 409 | 4992 | CGGAACTACATGCCCTAGGATATAA | Antisense | 2411 |
| 224588_at | 396 | 97 | 5173 | AGTCCTGTCCAGAAGATACATGCTT | Antisense | 2412 |
| 224588_at | 920 | 345 | 5191 | CATGCTTCCTGGCCTGATTGAGGAG | Antisense | 2413 |
| 224588_at | 310 | 197 | 5236 | ACAAGGTGTTGTGGTCTTCCATCCA | Antisense | 2414 |
| 224588_at | 979 | 977 | 5253 | TCCATCCAGTTTCTTAAGTGCTGAT | Antisense | 2415 |
| 224588_at | 1159 | 319 | 5297 | CACCTTGACCTGGCCTACAGAAGTA | Antisense | 2416 |
| 224588_at | 863 | 971 | 5338 | TGCCTCAGGCGTGCTTTTTGATTCA | Antisense | 2417 |
| 224588_at | 277 | 551 | 5390 | GAATATACCATTCTGGGTCCTGAGG | Antisense | 2418 |
| 224588_at | 46 | 655 | 5426 | GAGGGCATTAGATCACTGACAGCTG | Antisense | 2419 |
| 224588_at | 787 | 17 | 5490 | ATTTCAATGCCTATTCTCTGCAAGG | Antisense | 2420 |
| 224588_at | 89 | 1137 | 5503 | TTCTCTGCAAGGTACTATGTTTCGT | Antisense | 2421 |
| 224590_at | 229 | 1119 | 6809 | TTACAAGGTTTGTCTATTTTCAGTT | Antisense | 2422 |
| 224590_at | 45 | 105 | 6830 | AGTTCTTTACTTTTTACATGCTGAC | Antisense | 2423 |
| 224590_at | 76 | 315 | 6865 | CACTGCCTAAATAGATCTCTTTCAG | Antisense | 2424 |
| 224590_at | 720 | 293 | 6893 | CAATCCTCAGATAACGCATAGCAAA | Antisense | 2425 |
| 224590_at | 1144 | 687 | 6934 | GATTTCTCATGCAACAGCTTCTCTA | Antisense | 2426 |
| 224590_at | 834 | 325 | 6948 | CAGCTTCTCTAATTATACCTTAGAA | Antisense | 2427 |
| 224590_at | 988 | 1083 | 6961 | TATACCTTAGAAATGTTCTCCTTTT | Antisense | 2428 |
| 224590_at | 624 | 1107 | 6982 | TTTTTATCATCAAATCTGCTCAAGA | Antisense | 2429 |
| 224590_at | 1159 | 949 | 7039 | TGAAAACAGCTTAACATTTTACCAT | Antisense | 2430 |
| 224590_at | 306 | 15 | 7134 | ATTTCTCTAAATGTAGGCCTGGCTG | Antisense | 2431 |
| 224590_at | 930 | 373 | 7140 | CTAAATGTAGGCCTGGCTGGGCTTT | Antisense | 2432 |
| 224709_s_at | 1130 | 1031 | 3018 | TCAAGAGGTCATTTGTTCCCCATAG | Antisense | 2433 |
| 224709_s_at | 355 | 1143 | 3033 | TTCCCCATAGCAGCATATCTCATTT | Antisense | 2434 |
| 224709_s_at | 1122 | 1101 | 3084 | TTTACTACTCAACATTCATTATACT | Antisense | 2435 |
| 224709_s_at | 912 | 609 | 3161 | GACTTAGTCATGTGGATTGTTAGCA | Antisense | 2436 |
| 224709_s_at | 931 | 709 | 3179 | GTTAGCAGTGATCTGCATTCTGTAA | Antisense | 2437 |
| 224709_s_at | 819 | 657 | 3206 | GAGGTACTTTCCCATGATGTAGGCA | Antisense | 2438 |
| 224709_s_at | 593 | 91 | 3234 | AGTGGTGCCAGTAAGCGTAGAGCGG | Antisense | 2439 |
| 224709_s_at | 233 | 807 | 3282 | GGGTTTAGCATTTCCAGTGCAGCAT | Antisense | 2440 |
| 224709_s_at | 971 | 93 | 3297 | AGTGCAGCATTATCAGTGGGCCTTT | Antisense | 2441 |
| 224709_s_at | 238 | 1045 | 3410 | TAAAATGACTTCTGCTAAGCACCCA | Antisense | 2442 |
| 224709_s_at | 742 | 703 | 3510 | GTTCTGTGTTGGCTTGTAGATACTA | Antisense | 2443 |
| 224859_at | 928 | 535 | 1986 | GCACCCTGCGGTTTGCAGGGGGCTC | Antisense | 2444 |
| 224859_at | 1071 | 317 | 2077 | CACCCATAATTCTTACCCAGAGCAT | Antisense | 2445 |
| 224859_at | 313 | 525 | 2098 | GCATGGGGTTGGGGCGGAAACCTGG | Antisense | 2446 |
| 224859_at | 659 | 427 | 2138 | CCCTCGCCACGGCTAGAGAATCTGG | Antisense | 2447 |
| 224859_at | 389 | 909 | 2160 | TGGTGGTGTCCAAAATGTCTGTCCA | Antisense | 2448 |
| 224859_at | 335 | 427 | 2213 | CCCTCTGGACCTTTCATAGCAGCAG | Antisense | 2449 |
| 224859_at | 711 | 47 | 2272 | ATGCTTTGGGGACACCGAGGGGACT | Antisense | 2450 |
| 224859_at | 929 | 461 | 2309 | CCACCATGGTGCTATTCTGGGGCTG | Antisense | 2451 |
| 224859_at | 700 | 505 | 2365 | GCTCCTGGCCTCTGGTAGAGTGAGA | Antisense | 2452 |
| 224859_at | 685 | 609 | 2388 | GACTTCAGACGTTCTGATGCCTTCC | Antisense | 2453 |
| 224859_at | 707 | 1137 | 2399 | TTCTGATGCCTTCCGGATGTCATCT | Antisense | 2454 |
| 224940_s_at | 384 | 283 | 3317 | AATAAAACTGTGATCTCGTCTAGAG | Antisense | 2455 |
| 224940_s_at | 161 | 239 | 3322 | AACTGTGATCTCGTCTAGAGAAAAT | Antisense | 2456 |
| 224940_s_at | 675 | 1131 | 3350 | TTCATATTACAAACTGCTCTTCCAT | Antisense | 2457 |
| 224940_s_at | 287 | 1051 | 3357 | TACAAACTGCTCTTCCATATTTATG | Antisense | 2458 |
| 224940_s_at | 343 | 1105 | 3376 | TTTATGTACCATATTATACCTTTTT | Antisense | 2459 |
| 224940_s_at | 383 | 667 | 3440 | GATGTTGAAACCTGTTTGGCACCTT | Antisense | 2460 |
| 224940_s_at | 713 | 931 | 3442 | TGTTGAAACCTGTTTGGCACCTTCT | Antisense | 2461 |
| 224940_s_at | 665 | 719 | 3453 | GTTTGGCACCTTCTGGAAGCTACCA | Antisense | 2462 |
| 224940_s_at | 368 | 871 | 3457 | GGCACCTTCTGGAAGCTACCAAAAA | Antisense | 2463 |
| 224940_s_at | 293 | 225 | 3480 | AAAATGACACTCCATTGAAGTGCTT | Antisense | 2464 |
| 224940_s_at | 518 | 601 | 3485 | GACACTCCATTGAAGTGCTTAAAAG | Antisense | 2465 |
| 224941_at | 376 | 751 | 2955 | GTCTACTTAAGACTTCTGGTCATTT | Antisense | 2466 |
| 224941_at | 793 | 607 | 2965 | GACTTCTGGTCATTTCCAACTTATA | Antisense | 2467 |
| 224941_at | 210 | 975 | 3050 | TCCACATCAAGTGTTCCATTAAAAG | Antisense | 2468 |
| 224941_at | 61 | 257 | 3075 | AAGATAAGGCATTCTGAGTGCAAAC | Antisense | 2469 |
| 224941_at | 299 | 651 | 3090 | GAGTGCAAACAAATGGGGGCTTCTT | Antisense | 2470 |
| 224941_at | 1066 | 813 | 3105 | GGGGCTTCTTAAACTACACACCAGC | Antisense | 2471 |
| 224941_at | 178 | 235 | 3116 | AACTACACACCAGCAGTCAGTGAGG | Antisense | 2472 |
| 224941_at | 1093 | 287 | 3153 | AATTATTGAGTTGCTTTCTTGGGTC | Antisense | 2473 |
| 224941_at | 1027 | 645 | 3160 | GAGTTGCTTTCTTGGGTCTCTATAA | Antisense | 2474 |
| 224941_at | 1020 | 367 | 3179 | CTATAATCAATAACCTGTCTGCAGA | Antisense | 2475 |
| 224941_at | 178 | 1043 | 3189 | TAACCTGTCTGCAGATATCTATCTA | Antisense | 2476 |
| 225150_s_at | 1144 | 771 | 2283 | GTGAGAGAGAAAGGTGCTGGCATAG | Antisense | 2477 |
| 225150_s_at | 486 | 821 | 2295 | GGTGCTGGCATAGGATCTGCCCAGA | Antisense | 2478 |
| 225150_s_at | 1027 | 495 | 2298 | GCTGGCATAGGATCTGCCCAGAAGA | Antisense | 2479 |
| 225150_s_at | 657 | 567 | 2318 | GAAGAGAAAATGACCCATGCGCAGT | Antisense | 2480 |
| 225150_s_at | 687 | 57 | 2327 | ATGACCCATGCGCAGTTGGGCTCTG | Antisense | 2481 |
| 225150_s_at | 759 | 895 | 2335 | TGCGCAGTTGGGCTCTGGATACGGC | Antisense | 2482 |
| 225150_s_at | 603 | 997 | 2348 | TCTGGATACGGCGCTGTCTATAGCA | Antisense | 2483 |
| 225150_s_at | 417 | 887 | 2357 | GGCGCTGTCTATAGCAAGTTGGCCA | Antisense | 2484 |
| 225150_s_at | 211 | 369 | 2365 | CTATAGCAAGTTGGCCAGTCTGGCC | Antisense | 2485 |
| 225150_s_at | 480 | 425 | 2439 | CCCTCTTAAATTGTGGTTGCCATGG | Antisense | 2486 |
| 225150_s_at | 1018 | 1 | 2448 | ATTGTGGTTGCCATGGTACCGAGGG | Antisense | 2487 |
| 225230_at | 505 | 615 | 1075 | GACTGTTGTTGGTTATCTGGTGTGG | Antisense | 2488 |
| 225230_at | 399 | 767 | 1105 | GTGCACTTAGCATGCTGACTTGCTC | Antisense | 2489 |
| 225230_at | 286 | 609 | 1121 | GACTTGCTCATCAGTTTTGCACAGT | Antisense | 2490 |
| 225230_at | 610 | 389 | 1176 | CTCCATTGGAACCCCGAGGACAAAG | Antisense | 2491 |
| 225230_at | 924 | 361 | 1209 | CTTCACATGATCACTACTGCAGCAG | Antisense | 2492 |
| 225230_at | 346 | 169 | 1224 | ACTGCAGCAGAATGGTCTATGTCAT | Antisense | 2493 |
| 225230_at | 629 | 713 | 1261 | GTTTTTTCCTGACTTACATTCGTGA | Antisense | 2494 |
| 225230_at | 317 | 395 | 1345 | CTGCACCTTGCCCTATTAACAATGA | Antisense | 2495 |
| 225230_at | 830 | 949 | 1367 | TGAACGAACACGGCTACTTTCCAGA | Antisense | 2496 |
| 225230_at | 451 | 1127 | 1487 | TTCAACCACTTAATCAAGGCTGACA | Antisense | 2497 |
| 225230_at | 397 | 229 | 1601 | AACACCTATGCCTATACTTTTTTAT | Antisense | 2498 |
| 225475_at | 433 | 253 | 3904 | AAGACATTCCACTATTATGCGTAAT | Antisense | 2499 |
| 225475_at | 705 | 1079 | 3919 | TATGCGTAATGCTCAGCTTTTTGTA | Antisense | 2500 |
| 225475_at | 222 | 287 | 3992 | AATTTGTTCTTAATGCAGCCACTAT | Antisense | 2501 |
| 225475_at | 799 | 275 | 4003 | AATGCAGCCACTATAACTTGATAAG | Antisense | 2502 |
| 225475_at | 610 | 233 | 4017 | AACTTGATAAGTCATTGCACTATTT | Antisense | 2503 |
| 225475_at | 208 | 319 | 4172 | CACCCCCAACAAAGCACTATCATTT | Antisense | 2504 |
| 225475_at | 652 | 807 | 4208 | GGGTATTTTCTGTTTCATGATGTAT | Antisense | 2505 |
| 225475_at | 73 | 647 | 4242 | GAGTTCAGGATCTGCAATTCAGTTC | Antisense | 2506 |
| 225475_at | 914 | 547 | 4255 | GCAATTCAGTTCAACACAAGTTTGT | Antisense | 2507 |
| 225475_at | 838 | 925 | 4368 | TGTAAAATAATTCCCTTCTCCCATT | Antisense | 2508 |
| 225475_at | 819 | 1145 | 4391 | TTCCTTGTCTGCCATTCTGAATATG | Antisense | 2509 |
| 225531_at | 235 | 331 | 3557 | CAGAGATCTGTGGGGAAGCTCCGCC | Antisense | 2510 |
| 225531_at | 933 | 461 | 3581 | CCAGCCACACTCCTTGGGATAATAC | Antisense | 2511 |
| 225531_at | 987 | 35 | 3599 | ATAATACTAGCCGGTTCTGCCTGAT | Antisense | 2512 |
| 225531_at | 363 | 171 | 3659 | ACTCCTCTAGTGAGCCTTGACTGTT | Antisense | 2513 |
| 225531_at | 883 | 299 | 3705 | CAAGCCAAGAGGTTGCTGCAGCTGC | Antisense | 2514 |
| 225531_at | 955 | 471 | 3803 | GCCAGCCCAATGACACCTTGAAGTC | Antisense | 2515 |
| 225531_at | 757 | 1099 | 3839 | TTTCTTGCCCGCTAATAAAACCTAT | Antisense | 2516 |
| 225531_at | 201 | 979 | 3905 | TCCTTGGCATTTTGTTGTTCCGATC | Antisense | 2517 |
| 225531_at | 661 | 1153 | 3919 | TTGTTCCGATCACAGATTTCCCTGC | Antisense | 2518 |
| 225531_at | 15 | 811 | 3946 | GGGTGTCTGTGGTTATCAGCTGCAG | Antisense | 2519 |
| 225531_at | 492 | 163 | 4013 | ACGTGCCCTACCTTAGTGACGGGAG | Antisense | 2520 |
| 225736_at | 96 | 445 | 675 | CCTCTGGATATTGATGCCTCGGGTG | Antisense | 2521 |
| 225736_at | 1112 | 899 | 719 | TGGACACCGAATCCAGAGTGCCACT | Antisense | 2522 |
| 225736_at | 478 | 315 | 740 | CACTGTGCTCCTCAACGAGGACGTC | Antisense | 2523 |
| 225736_at | 837 | 671 | 768 | GATGAGAAGACTGCTGAGGCTGCGA | Antisense | 2524 |
| 225736_at | 83 | 939 | 782 | TGAGGCTGCGATGCAGCGCCTCAAA | Antisense | 2525 |
| 225736_at | 743 | 211 | 804 | AAAGCGGCCAACATTCCAGAGCATA | Antisense | 2526 |
| 225736_at | 596 | 631 | 822 | GAGCATAACACCATTGGCTTCATGT | Antisense | 2527 |
| 225736_at | 659 | 879 | 837 | GGCTTCATGTTTGCATGCGTTGGCA | Antisense | 2528 |
| 225736_at | 157 | 695 | 855 | GTTGGCAGGGGCTTTCAGTATTACA | Antisense | 2529 |
| 225736_at | 584 | 987 | 932 | TCCCTTATTCGGCTTCTTTGGAAAT | Antisense | 2530 |
| 225736_at | 225 | 71 | 1056 | ATGGCACTCATACATCTGGGGTCAT | Antisense | 2531 |
| 225882_at | 1108 | 535 | 2178 | GCACCTTACATATTTGATGCTCAGG | Antisense | 2532 |
| 225882_at | 944 | 461 | 2283 | CCACCCTTACATCTCCATAGTTGGT | Antisense | 2533 |
| 225882_at | 1067 | 695 | 2302 | GTTGGTACAGTTAGCTTGTAGCAGC | Antisense | 2534 |
| 225882_at | 561 | 51 | 2335 | ATGCCTGGAGATCACTGTCTGTTGG | Antisense | 2535 |
| 225882_at | 429 | 401 | 2349 | CTGTCTGTTGGTCTGATCTCAGTAT | Antisense | 2536 |
| 225882_at | 844 | 333 | 2368 | CAGTATCATACTGAGACACCTCCCC | Antisense | 2537 |
| 225882_at | 909 | 441 | 2391 | CCTGAGCCTTACCTACTTAAATTGG | Antisense | 2538 |
| 225882_at | 899 | 661 | 2453 | GAGGCAAGTTGCAAGGGAGCCAGAG | Antisense | 2539 |
| 225882_at | 841 | 285 | 2534 | AATTTCTTTGGCACATTGACTTACT | Antisense | 2540 |
| 225882_at | 169 | 873 | 2543 | GGCACATTGACTTACTGATATCTTT | Antisense | 2541 |
| 225882_at | 408 | 1001 | 2614 | TATTCCCCGGCGCAATAAAAATGCC | Antisense | 2542 |
| 226042_at | 697 | 1141 | 1640 | TTCCCTTCAGTCTTAAATTGTCTCC | Antisense | 2543 |
| 226042_at | 742 | 925 | 1688 | TGTAACCCTTGTGCTGTGTATTCTC | Antisense | 2544 |
| 226042_at | 853 | 1001 | 1719 | TCTGCTCACTCTCAGGGCCAAGCAG | Antisense | 2545 |
| 226042_at | 324 | 889 | 1756 | GGCCCTCTCTTGGCCAAAATCTGAG | Antisense | 2546 |
| 226042_at | 636 | 1161 | 1804 | TTGGTAGGTAGGTTCTGGCTGCCTG | Antisense | 2547 |
| 226042_at | 1006 | 919 | 1897 | TGTCCACTCCAGCAAGATCCAGGGA | Antisense | 2548 |
| 226042_at | 250 | 803 | 1918 | GGGATGATAGCCTTGCAGGGCCACT | Antisense | 2549 |
| 226042_at | 85 | 913 | 1942 | TGGGAGTTTGTGCCCAAGCTTCTCC | Antisense | 2550 |
| 226042_at | 345 | 565 | 2045 | GAAGTATGATTCCTCTGTCTTCAGT | Antisense | 2551 |
| 226042_at | 456 | 1021 | 2075 | TCAGGGGCATCCTGCCCATAGTACC | Antisense | 2552 |
| 226042_at | 1123 | 951 | 2129 | TGAAGCCTAGCACTCATGCAGCTCT | Antisense | 2553 |
| 226043_at | 966 | 89 | 2223 | AGTGGGGGGGTGGTCAGTGGAAGCT | Antisense | 2554 |
| 226043_at | 952 | 141 | 2244 | AGCTGGGGAGGCTTCACTCAGCTCA | Antisense | 2555 |
| 226043_at | 871 | 1027 | 2327 | TCACTGCTCTCTCCATGGGGAGGGG | Antisense | 2556 |
| 226043_at | 609 | 337 | 2357 | CAGGGCACCAAGTTCGGGATCCTAG | Antisense | 2557 |
| 226043_at | 79 | 435 | 2542 | CCCCACTCTCAGGCTAGGATGGAGA | Antisense | 2558 |
| 226043_at | 710 | 431 | 2667 | CCCCCCCGCATACTTGAATGTATGT | Antisense | 2559 |
| 226043_at | 11 | 727 | 2686 | GTATGTGCGTATTTATTGCTCACGT | Antisense | 2560 |
| 226043_at | 432 | 17 | 2696 | ATTTATTGCTCACGTCTGTGCCATG | Antisense | 2561 |
| 226043_at | 664 | 1027 | 2705 | TCACGTCTGTGCCATGTTGTCAATG | Antisense | 2562 |
| 226043_at | 534 | 931 | 2719 | TGTTGTCAATGGGTCCTTTCCAACC | Antisense | 2563 |
| 226043_at | 589 | 365 | 2734 | CTTTCCAACCCAAGAGGTACATTTG | Antisense | 2564 |
| 226118_at | 66 | 79 | 2108 | AGGAGACGGACCTGTGAGTCTGACC | Antisense | 2565 |
| 226118_at | 853 | 711 | 2180 | GTTTTGTCAGGTTGTCCTTGTTTGG | Antisense | 2566 |
| 226118_at | 459 | 451 | 2195 | CCTTGTTTGGATCCCTCAACTAGGT | Antisense | 2567 |
| 226118_at | 604 | 901 | 2249 | TGGACGTGTTTCTTTAACCTCATCC | Antisense | 2568 |
| 226118_at | 800 | 151 | 2330 | ACCAGAGCTTGGGACCAGGGCTCCT | Antisense | 2569 |
| 226118_at | 1000 | 287 | 2361 | AATTTTCTCTCCTGGTAGCTGAACA | Antisense | 2570 |
| 226118_at | 495 | 581 | 2410 | GAACAGGCTGCCGTCAGCCAGAGTT | Antisense | 2571 |
| 226118_at | 284 | 717 | 2453 | GTTTCCCTTGTTGACAATTGCTCTC | Antisense | 2572 |
| 226118_at | 1034 | 5 | 2469 | ATTGCTCTCCAGTTCCTATGAAAGC | Antisense | 2573 |
| 226118_at | 72 | 583 | 2520 | GAACACAACCATCTTAGGCCTGAGC | Antisense | 2574 |
| 226118_at | 662 | 1001 | 2581 | TCTGCTTGCCGAACTTTCTCAATAA | Antisense | 2575 |
| 226152_at | 97 | 829 | 533 | GGTAGTTTCTGAACCTATTGACATT | Antisense | 2576 |
| 226152_at | 41 | 839 | 567 | GGATCATGTGCCATATTTTGTTAGT | Antisense | 2577 |
| 226152_at | 194 | 245 | 759 | AACCAAATGTGAACTTCCTGTCTCC | Antisense | 2578 |
| 226152_at | 1009 | 445 | 786 | CCTCAGCACTAGTCCATGCCAGGAC | Antisense | 2579 |
| 226152_at | 1122 | 345 | 800 | CATGCCAGGACACCAGCTGACAATT | Antisense | 2580 |
| 226152_at | 412 | 499 | 815 | GCTGACAATTTCTTGGTTTTACTGT | Antisense | 2581 |
| 226152_at | 264 | 681 | 857 | GATCAATTACTGTCCTCACTTAGAA | Antisense | 2582 |
| 226152_at | 152 | 583 | 879 | GAACAAAGCCTGAGTCCGAGAATAT | Antisense | 2583 |
| 226152_at | 173 | 17 | 908 | ATTTTACCAATATATGCCTGTTACA | Antisense | 2584 |
| 226152_at | 241 | 285 | 1027 | AATATTTACCGACATTCCTTATACA | Antisense | 2585 |
| 226152_at | 855 | 947 | 1052 | TGACAGACACTTGGCTACATGGGAA | Antisense | 2586 |
| 226207_at | 234 | 461 | 1025 | CCAGCCGGAGTCGGGCATCAAGCGA | Antisense | 2587 |
| 226207_at | 931 | 711 | 1052 | GTTTAGCTTCTTCTCCCGAGATAAG | Antisense | 2588 |
| 226207_at | 628 | 989 | 1065 | TCCCGAGATAAGAAGCGCCTGGCCA | Antisense | 2589 |
| 226207_at | 319 | 435 | 1145 | CCGCGATGACGGTTACACAGAGCAA | Antisense | 2590 |
| 226207_at | 627 | 1031 | 1280 | TCAAGCCTCTCGGTGCAGATGCACC | Antisense | 2591 |
| 226207_at | 388 | 331 | 1295 | CAGATGCACCCTGAAAACTGACCCC | Antisense | 2592 |
| 226207_at | 924 | 215 | 1309 | AAACTGACCCCTCAAACAGACTGTC | Antisense | 2593 |
| 226207_at | 1150 | 1059 | 1391 | TACCCAGGGCTATCACTGTTTCTAA | Antisense | 2594 |
| 226207_at | 952 | 1067 | 1416 | TAGATGACTCTGATCCCGTAGGATA | Antisense | 2595 |
| 226207_at | 1154 | 663 | 1464 | GAGGCCAGACTTCTGCGTTAACTTC | Antisense | 2596 |
| 226207_at | 75 | 245 | 1507 | AAGCCAAATACATCACTTGCCACTA | Antisense | 2597 |
| 226470_at | 145 | 409 | 2343 | CTGGCCCCGAGCTTAGGGATGTGCT | Antisense | 2598 |
| 226470_at | 612 | 889 | 2345 | GGCCCCGAGCTTAGGGATGTGCTTG | Antisense | 2599 |
| 226470_at | 870 | 765 | 2363 | GTGCTTGCAAACCCTTCTCAAGGGT | Antisense | 2600 |
| 226470_at | 544 | 1135 | 2377 | TTCTCAAGGGTCTCACAACCCCAAC | Antisense | 2601 |
| 226470_at | 779 | 419 | 2396 | CCCAACATCTTCAGACTGGCCTGAC | Antisense | 2602 |
| 226470_at | 716 | 299 | 2398 | CAACATCTTCAGACTGGCCTGACCT | Antisense | 2603 |
| 226470_at | 726 | 1073 | 2477 | TAGGAATTTTTTAATGGACCATCAT | Antisense | 2604 |
| 226470_at | 532 | 853 | 2492 | GGACCATCATAGGGAGGGGGTGCTC | Antisense | 2605 |
| 226470_at | 89 | 351 | 2499 | CATAGGGAGGGGGTGCTCCTCTTTT | Antisense | 2606 |
| 226470_at | 238 | 381 | 2517 | CTCTTTTCCCACCAGGTTGAGGTGG | Antisense | 2607 |
| 226470_at | 30 | 1139 | 2547 | TTGCATCGGGGGTCCCCAGGGTATG | Antisense | 2608 |
| 226515_at | 943 | 359 | 633 | CTTCCTCGCAGCAAGCGGCTGGAGA | Antisense | 2609 |
| 226515_at | 304 | 413 | 695 | CGTCGCCGCTGACCTAGAGATGGCA | Antisense | 2610 |
| 226515_at | 573 | 153 | 706 | ACCTAGAGATGGCAGCCGGTCTCAC | Antisense | 2611 |
| 226515_at | 321 | 409 | 722 | CGGTCTCACCGACATATTTCAGCAT | Antisense | 2612 |
| 226515_at | 364 | 559 | 788 | GAATGGCAGACTCATGTGGCTCTAT | Antisense | 2613 |
| 226515_at | 121 | 1059 | 819 | TACTGGGAACTCGTTGTCGAACTGA | Antisense | 2614 |
| 226515_at | 700 | 513 | 905 | GCTTTTAGTGACTCGAGGCCAGGCA | Antisense | 2615 |
| 226515_at | 427 | 435 | 1020 | CCGCCACCGTCTGAGCAGAAGTGCA | Antisense | 2616 |
| 226515_at | 1109 | 1021 | 1053 | TCAGAGACAGAGGGTCTCCTCCCGA | Antisense | 2617 |
| 226515_at | 458 | 383 | 1080 | CTCTGCCGCTGTTGGGGATATGGTT | Antisense | 2618 |
| 226515_at | 179 | 721 | 1149 | GTATAACCTCGTATTTTAGCTCCAG | Antisense | 2619 |
| 226538_at | 345 | 1125 | 463 | TTAACCAGCAAGTTTCTGCTTTTTA | Antisense | 2620 |
| 226538_at | 955 | 35 | 563 | ATAATTTAATGTCTCTGGCACACAC | Antisense | 2621 |
| 226538_at | 944 | 307 | 581 | CACACACTAAAAACCATACACTTCA | Antisense | 2622 |
| 226538_at | 869 | 349 | 595 | CATACACTTCAGTTGTGATCTCAGT | Antisense | 2623 |
| 226538_at | 663 | 667 | 662 | GATGTTCAGTTGACCAAGTAGTTCA | Antisense | 2624 |
| 226538_at | 85 | 647 | 721 | GAGTTTGTGACTGCAGTGTTCAAGA | Antisense | 2625 |
| 226538_at | 699 | 125 | 743 | AGAACTCAGCATCCTTGTTTTCTAC | Antisense | 2626 |
| 226538_at | 122 | 15 | 811 | ATTATCATGATCTTCCCATGCCTTT | Antisense | 2627 |
| 226538_at | 1064 | 971 | 829 | TGCCTTTGTTGTACTTGTGCCGAAG | Antisense | 2628 |
| 226538_at | 700 | 775 | 853 | GTGTTTTGATATTCCTTTGTCTGGA | Antisense | 2629 |
| 226538_at | 676 | 1111 | 905 | TTTTGTTCACCTTGGAATCAACAGG | Antisense | 2630 |
| 226854_at | 474 | 207 | 91 | AAAGGATTTTGTGGAGTCGCCCCAG | Antisense | 2631 |
| 226854_at | 152 | 481 | 116 | GCCTGACGGCGTTAATTATCCCTGG | Antisense | 2632 |
| 226854_at | 854 | 1033 | 128 | TAATTATCCCTGGCTTTCATCTTGA | Antisense | 2633 |
| 226854_at | 662 | 357 | 148 | CTTGATGCTCTTGCAGGGGAGGCTC | Antisense | 2634 |
| 226854_at | 507 | 509 | 169 | GCTCAAGATGCCTTGTGGCTCGACC | Antisense | 2635 |
| 226854_at | 277 | 691 | 228 | GTTGAAACCTTGATCTGGGCCTGAC | Antisense | 2636 |
| 226854_at | 431 | 977 | 300 | TCCAGAGGGGGAGTCACTGCCACGA | Antisense | 2637 |
| 226854_at | 951 | 335 | 322 | CGAAGGTTGCCCAGAACTTTCCTTG | Antisense | 2638 |
| 226854_at | 295 | 391 | 347 | CTGCAAAAAGCCCCAAGACTTCTCC | Antisense | 2639 |
| 226854_at | 556 | 1147 | 390 | TTGCCATCGCGTTCTAAAAGCTGAC | Antisense | 2640 |
| 226854_at | 404 | 303 | 426 | CAACACTGTTCTCCCTGAGTAGGGT | Antisense | 2641 |
| 226971_at | 140 | 843 | 1991 | GGAGGCCTGAATACAGGCGGTCACC | Antisense | 2642 |
| 226971_at | 931 | 885 | 2006 | GGCGGTCACCTAAGGTGGTGCGTAC | Antisense | 2643 |
| 226971_at | 1050 | 907 | 2021 | TGGTGCGTACAGTGTTGGGCCACAT | Antisense | 2644 |
| 226971_at | 632 | 791 | 2037 | GGGCCACATGTCTAGTGGTGCCAGG | Antisense | 2645 |
| 226971_at | 879 | 613 | 2061 | GACTGAGAGATCACTTACCTCCACC | Antisense | 2646 |
| 226971_at | 790 | 637 | 2105 | GAGACTGATCCACAATGTTCCTGAG | Antisense | 2647 |
| 226971_at | 1064 | 293 | 2117 | CAATGTTCCTGAGGTGTCTTGGGCA | Antisense | 2648 |
| 226971_at | 267 | 779 | 2130 | GTGTCTTGGGCACCCATAGGCAGGT | Antisense | 2649 |
| 226971_at | 360 | 1075 | 2146 | TAGGCAGGTGACTTCTGGCTTAGGG | Antisense | 2650 |
| 226971_at | 528 | 879 | 2162 | GGCTTAGGGCAGGGTTCAGGAGGCT | Antisense | 2651 |
| 226971_at | 87 | 381 | 2202 | CTCTTTGGTCCCCACAGAACATGTT | Antisense | 2652 |
| 226972_s_at | 393 | 719 | 2224 | GTTTGGGTTGTGGAAGCCTATGGTA | Antisense | 2653 |
| 226972_s_at | 263 | 865 | 2235 | GGAAGCCTATGGTATTCTTGGCTAT | Antisense | 2654 |
| 226972_s_at | 708 | 1015 | 2250 | TCTTGGCTATTGCAGCTGTGGCTCT | Antisense | 2655 |
| 226972_s_at | 886 | 915 | 2266 | TGTGGCTCTGTATGTGTTACCCAAC | Antisense | 2656 |
| 226972_s_at | 870 | 917 | 2278 | TGTGTTACCCAACATGCGACAGCAG | Antisense | 2657 |
| 226972_s_at | 938 | 967 | 2292 | TGCGACAGCAGGAGTCAGAGTTCTG | Antisense | 2658 |
| 226972_s_at | 723 | 97 | 2304 | AGTCAGAGTTCTGCCTCATGGAGTG | Antisense | 2659 |
| 226972_s_at | 620 | 903 | 2322 | TGGAGTGATGGCAGACCTTGGCCAG | Antisense | 2660 |
| 226972_s_at | 512 | 325 | 2379 | CAGCTTTGGGCAGGACACACTGTGC | Antisense | 2661 |
| 226972_s_at | 51 | 585 | 2463 | GAAAAGGCTTGTGGGGAGCGGCTGA | Antisense | 2662 |
| 226972_s_at | 1065 | 983 | 2496 | TCCTGCCTTGTGTAAGAACCTGAGT | Antisense | 2663 |
| 227073_at | 738 | 1065 | 741 | TAGCATTAGCTTTACTTGGAGGGGA | Antisense | 2664 |
| 227073_at | 765 | 687 | 814 | GATTTGATGTATTCTTACTTTTCCA | Antisense | 2665 |
| 227073_at | 973 | 495 | 890 | GCTGGAGAGGCTTATGGGCAATATG | Antisense | 2666 |
| 227073_at | 605 | 563 | 914 | GAAGTGGGCCAAATTCTTGCAATAC | Antisense | 2667 |
| 227073_at | 394 | 9 | 926 | ATTCTTGCAATACACCCTATTCAAT | Antisense | 2668 |
| 227073_at | 215 | 1045 | 1058 | TAAAATTTGCTACCCTGAATTCTGA | Antisense | 2669 |
| 227073_at | 710 | 1149 | 1064 | TTGCTACCCTGAATTCTGAAATATT | Antisense | 2670 |
| 227073_at | 860 | 225 | 1105 | AAAATACTTTTAATGTCTGACATCT | Antisense | 2671 |
| 227073_at | 877 | 753 | 1119 | GTCTGACATCTCTTACATTTCACAT | Antisense | 2672 |
| 227073_at | 145 | 181 | 1133 | ACATTTCACATGGTTCTTTTTATTC | Antisense | 2673 |
| 227073_at | 1017 | 195 | 1233 | AAATTTACCCTTTGACATAATAATG | Antisense | 2674 |
| 227119_at | 790 | 95 | 266 | AGTGCCATGTAATTGTAGCTTGCTA | Antisense | 2675 |
| 227119_at | 341 | 1103 | 326 | TTTAACCATTTAACACTCTCTGCCA | Antisense | 2676 |
| 227119_at | 746 | 123 | 358 | AGAAAGTGGTTCTACTCTTACTGAA | Antisense | 2677 |
| 227119_at | 888 | 363 | 374 | CTTACTGAACACATTCTCTCTGACA | Antisense | 2678 |
| 227119_at | 1139 | 947 | 394 | TGACAAAATCACCAGCTGCTTTATT | Antisense | 2679 |
| 227119_at | 965 | 357 | 459 | CTTGACCAAAACTGCTCAGCTGAGA | Antisense | 2680 |
| 227119_at | 1115 | 333 | 577 | CAGTCCTGCCTTTTAAATCTTGTGG | Antisense | 2681 |
| 227119_at | 26 | 277 | 592 | AATCTTGTGGCAGCTCAGAAGGGAG | Antisense | 2682 |
| 227119_at | 554 | 695 | 674 | GATTGGTTACTGCTCACACATGATG | Antisense | 2683 |
| 227119_at | 883 | 23 | 710 | ATATATGCTTGTTTCCTTGCACCTG | Antisense | 2684 |
| 227119_at | 763 | 1095 | 758 | TTTCCTTCCCCTGTAGGCAATAAAT | Antisense | 2685 |
| 227135_at | 765 | 981 | 290 | TCCTCATTTAACTTTACAACCTTGC | Antisense | 2686 |
| 227135_at | 555 | 193 | 305 | ACAACCTTGCGAAGTGGGTCCAGGA | Antisense | 2687 |
| 227135_at | 718 | 99 | 353 | AGTAATGGGCATTGTCCTGCATTCC | Antisense | 2688 |
| 227135_at | 397 | 1041 | 408 | TAAGCTTCTATCCTGGCCTGAATAA | Antisense | 2689 |
| 227135_at | 1131 | 823 | 446 | GGTCTCAGAGATCAAGCCATATCCT | Antisense | 2690 |
| 227135_at | 580 | 453 | 462 | CCATATCCTCAGGCCTTATTTCCAT | Antisense | 2691 |
| 227135_at | 98 | 155 | 509 | ACCTTTGCTTCTCCGCTAATGAAAT | Antisense | 2692 |
| 227135_at | 662 | 943 | 533 | TGAGCTGGCAAGACCTCTGTTCATT | Antisense | 2693 |
| 227135_at | 391 | 579 | 611 | GAACTCTATTTCGAACCAAGCCTGT | Antisense | 2694 |
| 227135_at | 867 | 387 | 735 | CTCCTTCTTTCTCAATAGCTACTTT | Antisense | 2695 |
| 227135_at | 848 | 1063 | 750 | TAGCTACTTTCTTACTGCTTTTTGA | Antisense | 2696 |
| 227165_at | 323 | 859 | 2196 | GGACACCCAGTAGACCCTGAATTTC | Antisense | 2697 |
| 227165_at | 1101 | 1101 | 2217 | TTTCACTCTCTCTAAAAGGTTCTGA | Antisense | 2698 |
| 227165_at | 1127 | 443 | 2264 | CCTCCTGTGCACTGTTAGCTATGGC | Antisense | 2699 |
| 227165_at | 89 | 931 | 2276 | TGTTAGCTATGGCCACGGGAGCCTC | Antisense | 2700 |
| 227165_at | 627 | 459 | 2300 | CCAGAGCTGCCTGGTAGCTTCAGGT | Antisense | 2701 |
| 227165_at | 506 | 827 | 2322 | GGTTGACCTGCTTATCAGGCCTACG | Antisense | 2702 |
| 227165_at | 1001 | 889 | 2339 | GGCCTACGATCCTTCTGATTTAAGT | Antisense | 2703 |
| 227165_at | 790 | 853 | 2444 | GGACCTAGCATACCGTGGAGAACCA | Antisense | 2704 |
| 227165_at | 553 | 593 | 2574 | GAAAGTCAAATGGGGGACCTGTCCA | Antisense | 2705 |
| 227165_at | 74 | 761 | 2594 | GTCCATTGTGCTCTATTAATCTTGT | Antisense | 2706 |
| 227165_at | 970 | 197 | 2640 | AAATTTGAGTTTGTCCTTGTTCTAG | Antisense | 2707 |
| 227263_at | 864 | 235 | 607 | AACTCACTCATTGTCAGGTGTCCGT | Antisense | 2708 |
| 227263_at | 165 | 779 | 624 | GTGTCCGTGGAGTGTTTTTGGCATG | Antisense | 2709 |
| 227263_at | 249 | 1091 | 640 | TTTGGCATGGTGACCTGTCTGGGCC | Antisense | 2710 |
| 227263_at | 5 | 793 | 660 | GGGCCCAGCATGTTGCAGATGTGTA | Antisense | 2711 |
| 227263_at | 522 | 725 | 698 | GTATGCATATCTCTGTGTGACTGTC | Antisense | 2712 |
| 227263_at | 568 | 1149 | 727 | TTGCAAGCTGGCTGGATCCAACCAT | Antisense | 2713 |
| 227263_at | 684 | 811 | 789 | GGGGGAGGTCACTGCAGAAGGATGG | Antisense | 2714 |
| 227263_at | 1080 | 69 | 810 | ATGGAACTGACCTTTATTCCCCAGT | Antisense | 2715 |
| 227263_at | 478 | 437 | 928 | CCGGACTCCTGAAGACTGCACAAGG | Antisense | 2716 |
| 227263_at | 661 | 383 | 1013 | CTCTGCTCCGTACTAGCGTTTACAG | Antisense | 2717 |
| 227263_at | 24 | 971 | 1056 | TGCCTGTACTAGTTTTTAGACCCCA | Antisense | 2718 |
| 227265_at | 1000 | 181 | 1401 | ACATCTGCTAGAACCTTTTGCCTTA | Antisense | 2719 |
| 227265_at | 885 | 449 | 1414 | CCTTTTGCCTTAACTATTCACCAAT | Antisense | 2720 |
| 227265_at | 319 | 13 | 1480 | ATTAGAATCTTGTCTTGGTTCCATT | Antisense | 2721 |
| 227265_at | 779 | 665 | 1507 | GATGGCTAATATTTGTTATCTTAAT | Antisense | 2722 |
| 227265_at | 325 | 19 | 1539 | ATTTCTGAGGTCATGATTACTTGAA | Antisense | 2723 |
| 227265_at | 864 | 1 | 1565 | ATATTGACTAAAACTGGGTCCTTAG | Antisense | 2724 |
| 227265_at | 290 | 805 | 1580 | GGGTCCTTAGAAATTCCAGGTGGAG | Antisense | 2725 |
| 227265_at | 128 | 789 | 1599 | GTGGAGCTGATTTACCTATGACTGA | Antisense | 2726 |
| 227265_at | 512 | 289 | 1671 | AATTAATGACACATCTGTTCAATAA | Antisense | 2727 |
| 227265_at | 1066 | 739 | 1802 | GTAATGTCATCACAATAAGCTCTTG | Antisense | 2728 |
| 227265_at | 778 | 277 | 1852 | AATTAGGTTTATGTTTCATGTCTTT | Antisense | 2729 |
| 227908_at | 38 | 1013 | 441 | TCTTCACCCTACAACATCTATGTTA | Antisense | 2730 |
| 227908_at | 1085 | 223 | 596 | AAAATGCCTCCCACAATTGCAGACT | Antisense | 2731 |
| 227908_at | 517 | 197 | 608 | ACAATTGCAGACTCACTCATTTTAC | Antisense | 2732 |
| 227908_at | 884 | 1153 | 646 | TTGTCAACACCTTTTCTTGGGACCA | Antisense | 2733 |
| 227908_at | 240 | 261 | 679 | AAGTCAGTGGATACCGTGTCCTGTA | Antisense | 2734 |
| 227908_at | 613 | 779 | 694 | GTGTCCTGTAGACATGTCACTTGTG | Antisense | 2735 |
| 227908_at | 978 | 267 | 721 | AAGGCACGGACAACATGAACTGGCA | Antisense | 2736 |
| 227908_at | 775 | 931 | 811 | TGATTTTACCTCTTAGTTCTTACGA | Antisense | 2737 |
| 227908_at | 374 | 561 | 834 | GAAGGATGGTTATCGGGCTGGAAAA | Antisense | 2738 |
| 227908_at | 1049 | 167 | 870 | ACTGATGCTTTGGAGCCTCTCAATG | Antisense | 2739 |
| 227908_at | 426 | 687 | 934 | GATTTGCAGCACAATCCAACTTTGA | Antisense | 2740 |
| 227927_at | 779 | 1061 | 1233 | TACCTCTGTCGACCTGTGAAACTGA | Antisense | 2741 |
| 227927_at | 587 | 445 | 1235 | CCTCTGTCGACCTGTGAAACTGAAC | Antisense | 2742 |
| 227927_at | 525 | 589 | 1250 | GAAACTGAACAAACAGCTTATCTGC | Antisense | 2743 |
| 227927_at | 631 | 515 | 1265 | GCTTATCTGCCCCTAATGTGAAATG | Antisense | 2744 |
| 227927_at | 230 | 365 | 1266 | CTTATCTGCCCCTAATGTGAAATGA | Antisense | 2745 |
| 227927_at | 753 | 45 | 1269 | ATCTGCCCCTAATGTGAAATGATGG | Antisense | 2746 |
| 227927_at | 467 | 485 | 1273 | GCCCCTAATGTGAAATGATGGGACA | Antisense | 2747 |
| 227927_at | 867 | 277 | 1400 | AATCCAGCAGGAGTTTCTTAGTTAG | Antisense | 2748 |
| 227927_at | 1130 | 715 | 1412 | GTTTCTTAGTTAGGATCCACAGCCT | Antisense | 2749 |
| 227927_at | 811 | 363 | 1416 | CTTAGTTAGGATCCACAGCCTGGGA | Antisense | 2750 |
| 227927_at | 909 | 999 | 1483 | TCTGCATTTCTTGAAACCATTATTA | Antisense | 2751 |
| 227929_at | 179 | 647 | 2213 | GAGTTCATGTGTCTAATCATTATCT | Antisense | 2752 |
| 227929_at | 758 | 37 | 2364 | ATCACATCAGTAGCAAACACAGGGT | Antisense | 2753 |
| 227929_at | 959 | 309 | 2381 | CACAGGGTGGGAACTCAATCTTATG | Antisense | 2754 |
| 227929_at | 804 | 279 | 2397 | AATCTTATGTTATAGCTTACTCTTA | Antisense | 2755 |
| 227929_at | 73 | 287 | 2487 | AATATTTCAGTGACTTTTCAGATTT | Antisense | 2756 |
| 227929_at | 899 | 687 | 2507 | GATTTATTTCTTGTTAGCCGCTGTG | Antisense | 2757 |
| 227929_at | 434 | 145 | 2522 | AGCCGCTGTGTCTATTTGGTGCTAC | Antisense | 2758 |
| 227929_at | 1037 | 1109 | 2658 | TTTTCTCACCATTTGCATGTACATC | Antisense | 2759 |
| 227929_at | 495 | 181 | 2678 | ACATCATGTCTTTCCAATTGTTTTC | Antisense | 2760 |
| 227929_at | 34 | 1159 | 2729 | TTGTGCAGTGGTTCAACCTGACCAA | Antisense | 2761 |
| 227929_at | 449 | 787 | 2755 | GTGGTATTATGCTGCTGGGAAGTAA | Antisense | 2762 |
| 228017_s_at | 378 | 479 | 799 | GCCTGCGTAAGTGAGGAAACAGCTG | Antisense | 2763 |
| 228017_s_at | 374 | 939 | 810 | TGAGGAAACAGCTGATCCTGCTCCT | Antisense | 2764 |
| 228017_s_at | 441 | 377 | 847 | CTCAGCGACCGACCAGTGACAATGA | Antisense | 2765 |
| 228017_s_at | 979 | 151 | 858 | ACCAGTGACAATGACAGGAGCTCCC | Antisense | 2766 |
| 228017_s_at | 337 | 769 | 862 | GTGACAATGACAGGAGCTCCCAGGC | Antisense | 2767 |
| 228017_s_at | 1106 | 383 | 933 | CTGCCCTGGGTTCTAAGTACTGGAC | Antisense | 2768 |
| 228017_s_at | 317 | 487 | 935 | GCCCTGGGTTCTAAGTACTGGACAC | Antisense | 2769 |
| 228017_s_at | 154 | 807 | 940 | GGGTTCTAAGTACTGGACACCAGCC | Antisense | 2770 |
| 228017_s_at | 48 | 317 | 983 | CACGGCTGGCTGCAGCGTCAAGAGA | Antisense | 2771 |
| 228017_s_at | 987 | 495 | 987 | GCTGGCTGCAGCGTCAAGAGAGTTT | Antisense | 2772 |
| 228017_s_at | 1105 | 501 | 991 | GCTGCAGCGTCAAGAGAGTTTGTAA | Antisense | 2773 |
| 228054_at | 355 | 239 | 979 | AACGGAATACCTGCTAGGTTCCAGG | Antisense | 2774 |
| 228054_at | 880 | 709 | 1038 | GTTAGATCTCAGCAGAGCCCATTCT | Antisense | 2775 |
| 228054_at | 1043 | 143 | 1053 | AGCCCATTCTGCAAGACCTGGCTGA | Antisense | 2776 |
| 228054_at | 184 | 193 | 1123 | ACACAGGAACCGAGACCTGGCTTCC | Antisense | 2777 |
| 228054_at | 886 | 855 | 1193 | GGACTGTGTGGCCAACCAAGTGCTT | Antisense | 2778 |
| 228054_at | 689 | 305 | 1242 | CAAAGCAAACTGCTCCGGGTACCAG | Antisense | 2779 |
| 228054_at | 837 | 443 | 1272 | CCTCCAACCTGGCAAGGGTGTGCGC | Antisense | 2780 |
| 228054_at | 10 | 795 | 1304 | GGGCTTGCAGGTGAGGGGGCTGTAT | Antisense | 2781 |
| 228054_at | 425 | 813 | 1319 | GGGGCTGTATGCTTCAGGAACTAAC | Antisense | 2782 |
| 228054_at | 244 | 1031 | 1383 | TCAAGCACAGCAATCCCATTTGGGA | Antisense | 2783 |
| 228054_at | 234 | 817 | 1434 | GGTAAGGGCGCATGGAATGGGGCTA | Antisense | 2784 |
| 228071_at | 1072 | 79 | 679 | AGGAGTTGGTGGAGCTGATAGAGAA | Antisense | 2785 |
| 228071_at | 259 | 821 | 707 | GGTGCAGTGCAACGAAGGGGCTTAC | Antisense | 2786 |
| 228071_at | 2 | 951 | 714 | TGCAACGAAGGGGCTTACTTTTCTG | Antisense | 2787 |
| 228071_at | 410 | 1137 | 734 | TTCTGATGACATATACAAGGACACA | Antisense | 2788 |
| 228071_at | 563 | 661 | 791 | GAGGAAAATCTACACTGACCAATTA | Antisense | 2789 |
| 228071_at | 447 | 27 | 964 | ATAGGATTTGGAAGATGCTTTCAGA | Antisense | 2790 |
| 228071_at | 15 | 597 | 987 | GAAATATGGCATAGGTTTTTGTCGA | Antisense | 2791 |
| 228071_at | 260 | 877 | 994 | GGCATAGGTTTTTGTCGAAATGTAA | Antisense | 2792 |
| 228071_at | 651 | 1107 | 1020 | TTTTATTCTTCCTAATTTACTGTGA | Antisense | 2793 |
| 228071_at | 603 | 1013 | 1026 | TCTTCCTAATTTACTGTGATTTGTT | Antisense | 2794 |
| 228071_at | 599 | 681 | 1075 | GATAGTTAGAGAAATACCTCCTTCC | Antisense | 2795 |
| 228094_at | 149 | 803 | 193 | GGGAGAAACACATTTACTCCCCAAT | Antisense | 2796 |
| 228094_at | 1102 | 373 | 272 | CTACATGACCATGCACCCAGTTTGG | Antisense | 2797 |
| 228094_at | 156 | 85 | 306 | AGGTCAGATCGGAACAACTCACTTG | Antisense | 2798 |
| 228094_at | 371 | 559 | 381 | GAATGGAGAGTCCCTTCATCTCAGC | Antisense | 2799 |
| 228094_at | 758 | 1023 | 401 | TCAGCAGCGGTGGAGACTCTCTCCT | Antisense | 2800 |
| 228094_at | 352 | 381 | 443 | CTCTACCAGTGATTTCAGACTCCCG | Antisense | 2801 |
| 228094_at | 1 | 667 | 514 | GATGGAGAATTTGGAGCCTGGCAGA | Antisense | 2802 |
| 228094_at | 364 | 109 | 538 | AGAGACTGGACAGCTCTGGAGGAAC | Antisense | 2803 |
| 228094_at | 295 | 997 | 552 | TCTGGAGGAACAGGCCTGCTGAGGG | Antisense | 2804 |
| 228094_at | 144 | 891 | 594 | GGCCTCTGGAGTGGGACACTGGCCC | Antisense | 2805 |
| 228094_at | 640 | 439 | 671 | CCTGTGGGCAGGGTTCTTAGTGGAT | Antisense | 2806 |
| 228128_x_at | 382 | 667 | 281 | GATGTTGAAACCTGTTTGGCACCTT | Antisense | 2807 |
| 228128_x_at | 666 | 719 | 294 | GTTTGGCACCTTCTGGAAGCTACCA | Antisense | 2808 |
| 228128_x_at | 1075 | 349 | 358 | CATAAGAATTCTACTGGCCTATTGT | Antisense | 2809 |
| 228128_x_at | 690 | 219 | 447 | AAACACAATTCCAATCCTTTTTCTG | Antisense | 2810 |
| 228128_x_at | 199 | 839 | 529 | GGATCTAAGTTAAATCTTTTGGCAA | Antisense | 2811 |
| 228128_x_at | 341 | 173 | 661 | ACTCAGACATATTTTGCTGTTCATG | Antisense | 2812 |
| 228128_x_at | 6 | 441 | 696 | CCTGTTCTCATGGATTTGTTTTCCC | Antisense | 2813 |
| 228128_x_at | 714 | 687 | 708 | GATTTGTTTTCCCATACTGTTTTCT | Antisense | 2814 |
| 228128_x_at | 157 | 351 | 720 | CATACTGTTTTCTCTGATCTCAATT | Antisense | 2815 |
| 228128_x_at | 996 | 43 | 736 | ATCTCAATTACAGGTTGGATCTCAC | Antisense | 2816 |
| 228128_x_at | 618 | 15 | 786 | ATTTTGCCACTGTTGATTACTATAC | Antisense | 2817 |
| 228492_at | 976 | 243 | 132 | AACCAATACACAAAATTTTCCTATG | Antisense | 2818 |
| 228492_at | 476 | 15 | 146 | ATTTTCCTATGTCAGAATGTGGTGG | Antisense | 2819 |
| 228492_at | 677 | 21 | 186 | ATTTGGTGTGCTTGCGATTTTTTTT | Antisense | 2820 |
| 228492_at | 1018 | 1135 | 250 | TTCTCCTGATCCCGGTGAAGTGTAC | Antisense | 2821 |
| 228492_at | 861 | 675 | 257 | GATCCCGGTGAAGTGTACATCATAA | Antisense | 2822 |
| 228492_at | 254 | 93 | 268 | AGTGTACATCATAAGAATCCATAGT | Antisense | 2823 |
| 228492_at | 298 | 1055 | 292 | TACTTTGAAGTACCATTGCACCAAG | Antisense | 2824 |
| 228492_at | 189 | 153 | 303 | ACCATTGCACCAAGATGTCTGACTG | Antisense | 2825 |
| 228492_at | 543 | 753 | 319 | GTCTGACTGAATTCATAGTCACACT | Antisense | 2826 |
| 228492_at | 949 | 1039 | 419 | TAAGATCTAAGCTCCTGCTTATTTA | Antisense | 2827 |
| 228492_at | 2 | 683 | 595 | GATACAGCCTATCCTATACTATTTA | Antisense | 2828 |
| 228565_at | 474 | 1077 | 464 | TATGTCCTCAGTTTTACTTCTACCA | Antisense | 2829 |
| 228565_at | 1148 | 219 | 488 | AAAACATCCCTGTATGTGTGTGCAT | Antisense | 2830 |
| 228565_at | 1048 | 781 | 565 | GTGTGTGTTGGGGAGTGTATGTGAT | Antisense | 2831 |
| 228565_at | 791 | 777 | 579 | GTGTATGTGATCTGGGTGTTTGTTT | Antisense | 2832 |
| 228565_at | 905 | 929 | 599 | TGTTTATCTCTGTTATTATTCCCCT | Antisense | 2833 |
| 228565_at | 980 | 365 | 622 | CTTTAGCTTTATTTTAGTCAACTCT | Antisense | 2834 |
| 228565_at | 641 | 289 | 699 | AATTCAATCTCACATGTTACTGCAG | Antisense | 2835 |
| 228565_at | 206 | 685 | 723 | GATAGTTAACTTTTGCTGCAATCTA | Antisense | 2836 |
| 228565_at | 930 | 225 | 853 | AAAATTAAGGTACTCTCACTGCAGA | Antisense | 2837 |
| 228565_at | 979 | 269 | 883 | AAGGCCTGGGCCTAATGTGCTGTAT | Antisense | 2838 |
| 228565_at | 1140 | 763 | 899 | GTGCTGTATTATGAAGCCTTGTGAC | Antisense | 2839 |
| 228604_at | 845 | 593 | 622 | GAAATGGTGGTTTCCTTCTGATAAG | Antisense | 2840 |
| 228604_at | 731 | 853 | 656 | GGACCATGTCGCTTATATATCACCC | Antisense | 2841 |
| 228604_at | 1139 | 415 | 665 | CGCTTATATATCACCCTACTAGGGG | Antisense | 2842 |
| 228604_at | 898 | 685 | 689 | GATATAATTTTCCCTTCTAAGCAAG | Antisense | 2843 |
| 228604_at | 313 | 271 | 717 | AATGGCAGTGCTAAATAGTTTTGTA | Antisense | 2844 |
| 228604_at | 299 | 287 | 785 | AATATTTTTCAGATGCATTTGCATT | Antisense | 2845 |
| 228604_at | 637 | 855 | 868 | GGACTGAAATTCTGTTACATGATGT | Antisense | 2846 |
| 228604_at | 585 | 23 | 954 | ATATAAAGCAAATTCACACGTTTTC | Antisense | 2847 |
| 228604_at | 865 | 589 | 1011 | GAAACCTCAGTCTTGAAATTTTGAT | Antisense | 2848 |
| 228604_at | 786 | 219 | 1040 | AAAAATCATGACACTGTTTTACCAT | Antisense | 2849 |
| 228604_at | 882 | 645 | 1071 | GAGTAGCTAACTTTTGGTAACACCT | Antisense | 2850 |
| 228812_at | 843 | 637 | 765 | GAGACTTCATTGGTGATACACTCAA | Antisense | 2851 |
| 228812_at | 406 | 775 | 777 | GTGATACACTCAATTTTTACTGGGT | Antisense | 2852 |
| 228812_at | 488 | 509 | 807 | GCTAATAATGTTGGTCACTGTCTCA | Antisense | 2853 |
| 228812_at | 742 | 301 | 869 | CACAAAGCTTTTGGGTAACCAGCGT | Antisense | 2854 |
| 228812_at | 873 | 805 | 881 | GGGTAACCAGCGTTCTTAAATGTAT | Antisense | 2855 |
| 228812_at | 552 | 1075 | 903 | TATGGTTTTTGACCAGGTGAACCCT | Antisense | 2856 |
| 228812_at | 775 | 623 | 913 | GACCAGGTGAACCCTTTAGAAGTGA | Antisense | 2857 |
| 228812_at | 1155 | 199 | 963 | AAATACCTTTGGCTGTGATGAATGT | Antisense | 2858 |
| 228812_at | 1101 | 953 | 981 | TGAATGTAGATCCCAGCAGAATACC | Antisense | 2859 |
| 228812_at | 137 | 1153 | 1021 | TTGACTGAGTATTTGTAGATGCTTA | Antisense | 2860 |
| 228812_at | 198 | 259 | 1091 | AAGTTCTCAGGTACTGTTCAATTAT | Antisense | 2861 |
| 229022_at | 446 | 587 | 826 | GAAACACTTTTAGAGCACTTGAGGA | Antisense | 2862 |
| 229022_at | 167 | 27 | 937 | ATAGGACCCCAGACAGTTTATACCT | Antisense | 2863 |
| 229022_at | 124 | 715 | 952 | GTTTATACCTTCCATTTGCTGTTTT | Antisense | 2864 |
| 229022_at | 697 | 263 | 979 | AAGTGTGAGCCTGCCAAGTCAACAA | Antisense | 2865 |
| 229022_at | 990 | 479 | 987 | GCCTGCCAAGTCAACAAGTATGCCT | Antisense | 2866 |
| 229022_at | 645 | 297 | 1001 | CAAGTATGCCTTTAGCGCACATGTA | Antisense | 2867 |
| 229022_at | 328 | 311 | 1018 | CACATGTAAATAGCCTGCACTTCCT | Antisense | 2868 |
| 229022_at | 744 | 539 | 1034 | GCACTTCCTAAATCTCGTGTGGCCT | Antisense | 2869 |
| 229022_at | 835 | 15 | 1195 | ATTTTGCCACATTCTGTAACTGTTT | Antisense | 2870 |
| 229022_at | 529 | 1101 | 1256 | TTTCATCTTTTGTTTCGTGTATATA | Antisense | 2871 |
| 229022_at | 845 | 777 | 1272 | GTGTATATACTGTTTGCCTTTTTCA | Antisense | 2872 |
| 229026_at | 953 | 205 | 268 | AAAGGCCCACTGATAATGCTGCACT | Antisense | 2873 |
| 229026_at | 20 | 465 | 352 | CCACTTCATGCCTACATCATGGGAA | Antisense | 2874 |
| 229026_at | 222 | 531 | 396 | GCAGATCACTGCTAACAATCCACAT | Antisense | 2875 |
| 229026_at | 1109 | 1129 | 430 | TTCAGTTTCCCTTGCTTAACATTTG | Antisense | 2876 |
| 229026_at | 85 | 225 | 504 | AAAATCCTATTTAATTCGCTTCAAT | Antisense | 2877 |
| 229026_at | 1038 | 943 | 562 | TGACCTCTTGAGATTTACAACCCCC | Antisense | 2878 |
| 229026_at | 24 | 1015 | 669 | TCTTGATTTTATTGGCACCTCCCAG | Antisense | 2879 |
| 229026_at | 550 | 341 | 691 | CAGGCACTTTGGCATGTGCTCCTGT | Antisense | 2880 |
| 229026_at | 1136 | 777 | 721 | GTGTAGTTACTGTGCCTGTAGAGTT | Antisense | 2881 |
| 229026_at | 946 | 597 | 774 | GACAAGTCCACTTTTGGCTTACCAT | Antisense | 2882 |
| 229026_at | 340 | 343 | 796 | CATGGAGATCTGCTCCAACACATGC | Antisense | 2883 |
| 229047_at | 140 | 441 | 13 | CCTGTGATGATAGCTTCTTCCCATG | Antisense | 2884 |
| 229047_at | 182 | 919 | 15 | TGTGATGATAGCTTCTTCCCATGTG | Antisense | 2885 |
| 229047_at | 968 | 683 | 21 | GATAGCTTCTTCCCATGTGGGGATT | Antisense | 2886 |
| 229047_at | 1132 | 515 | 25 | GCTTCTTCCCATGTGGGGATTAAGG | Antisense | 2887 |
| 229047_at | 375 | 841 | 36 | TGTGGGGATTAAGGGTAGGGAGTGG | Antisense | 2888 |
| 229047_at | 728 | 557 | 64 | GAATGGTCCAAACCCGGAAGGAGCT | Antisense | 2889 |
| 229047_at | 723 | 821 | 68 | GGTCCAAACCCGGAAGGAGCTAATG | Antisense | 2890 |
| 229047_at | 132 | 851 | 83 | GGAGCTAATGGAGAGGATAGGGCCA | Antisense | 2891 |
| 229047_at | 43 | 89 | 101 | AGGGCCAAAGTGGGACAGGGCTTGG | Antisense | 2892 |
| 229047_at | 1146 | 85 | 109 | AGTGGGACAGGGCTTGGATGGTAGA | Antisense | 2893 |
| 229047_at | 898 | 919 | 427 | TGTAGACGCGCACCGGGGTGGAGTT | Antisense | 2894 |
| 229094_at | 421 | 987 | 236 | TCCCCTAGTGAAATCCATCTGTCCT | Antisense | 2895 |
| 229094_at | 577 | 51 | 248 | ATCCATCTGTCCTGAAGGGAACCCT | Antisense | 2896 |
| 229094_at | 1039 | 317 | 323 | CACCAAAGGGAGGCCGCACGGATTC | Antisense | 2897 |
| 229094_at | 573 | 649 | 432 | GAGTAAGACTGAGTCTCCTCACCGA | Antisense | 2898 |
| 229094_at | 78 | 417 | 454 | CGAAGAGATCTGGCTGTATCCCGAG | Antisense | 2899 |
| 229094_at | 134 | 723 | 469 | GTATCCCGAGTCTCTGAAACCTTTG | Antisense | 2900 |
| 229094_at | 518 | 67 | 526 | ATGTGGAGCCGCAAGGGCTCGCCAG | Antisense | 2901 |
| 229094_at | 243 | 883 | 541 | GGCTCGCCAGAGGGATCTCAGGAGC | Antisense | 2902 |
| 229094_at | 17 | 853 | 576 | GGAGCGCTGTTGAGTGACAGGTCTG | Antisense | 2903 |
| 229094_at | 674 | 605 | 591 | GACAGGTCTGATGCAGTAGCTTTTA | Antisense | 2904 |
| 229094_at | 867 | 591 | 645 | GAAAGTCTACCTTTCTAAGTTCAGT | Antisense | 2905 |
| 229308_at | 791 | 625 | 305 | GAGCCATAAAATTGAAGCCAGTAGC | Antisense | 2906 |
| 229308_at | 880 | 293 | 335 | CAATATGGAGGCCTTCTTACTGTAG | Antisense | 2907 |
| 229308_at | 1004 | 725 | 356 | GTAGGGAAGAGGTGTCTAGCTTAGA | Antisense | 2908 |
| 229308_at | 730 | 371 | 396 | CTAGATGAGATTACCCATAGCAGGG | Antisense | 2909 |
| 229308_at | 167 | 955 | 425 | TGAATCATCACCTACCAAAGACTGT | Antisense | 2910 |
| 229308_at | 942 | 251 | 442 | AAGACTGTACGGAAAGGTGTGTTAT | Antisense | 2911 |
| 229308_at | 219 | 521 | 477 | GCATTTTAGTTTGCACACATGTAAA | Antisense | 2912 |
| 229308_at | 193 | 139 | 538 | AGCTCCGCATCCTAAGACTTAAAAC | Antisense | 2913 |
| 229308_at | 675 | 547 | 565 | GCAATTCCTATGCAAATCATCTGCT | Antisense | 2914 |
| 229308_at | 18 | 347 | 582 | CATCTGCTTAACTGAACTACAAACG | Antisense | 2915 |
| 229308_at | 609 | 373 | 598 | CTACAAACGTTTTTCATTAATTCAT | Antisense | 2916 |
| 229575_at | 840 | 559 | 86 | GAAGGCTATGAGTTGCTGCTGGCCC | Antisense | 2917 |
| 229575_at | 1110 | 1057 | 151 | TACTGGTACACTTACATTTCTTGGA | Antisense | 2918 |
| 229575_at | 345 | 533 | 179 | GCAGCAGAAACCTTGGGATATCCTT | Antisense | 2919 |
| 229575_at | 358 | 355 | 201 | CTTGTGGGTCAAGGAGGCAACTCTC | Antisense | 2920 |
| 229575_at | 409 | 389 | 223 | CTCCTCACGGATTCCCTGATTTGAT | Antisense | 2921 |
| 229575_at | 777 | 275 | 271 | AATCCAAGTTTTCACAAGTTTCTCT | Antisense | 2922 |
| 229575_at | 564 | 447 | 315 | CCTAGACCAAGAATTCCTACCACGC | Antisense | 2923 |
| 229575_at | 845 | 1143 | 328 | TTCCTACCACGCAAGGCCTGTGAAA | Antisense | 2924 |
| 229575_at | 405 | 675 | 391 | GATGCCAAAGTACCTGTTTTTATAA | Antisense | 2925 |
| 229575_at | 799 | 293 | 469 | CAATGATTTTTCTGCAGTACCTTAA | Antisense | 2926 |
| 229575_at | 86 | 291 | 524 | AATTCTGCCATGTAGTGCCTTATTT | Antisense | 2927 |
| 229598_at | 248 | 15 | 566 | ATTTTTCTTAGCTAAATCTGGCAAC | Antisense | 2928 |
| 229598_at | 573 | 133 | 636 | AGCAGAATCTGCCATATGAGTAATA | Antisense | 2929 |
| 229598_at | 259 | 649 | 653 | GAGTAATAGAAGTGAGCAGGCCCAG | Antisense | 2930 |
| 229598_at | 304 | 335 | 675 | CAGGACTCCCTAAGTCAAGAAACCA | Antisense | 2931 |
| 229598_at | 19 | 121 | 692 | AGAAACCAAGAGGCGTCATTACGGA | Antisense | 2932 |
| 229598_at | 549 | 209 | 717 | AAAGAGTAACTCACCCTGTGTGCTC | Antisense | 2933 |
| 229598_at | 90 | 143 | 759 | AGCGATGCCCCCATGTTATGAATGG | Antisense | 2934 |
| 229598_at | 747 | 859 | 784 | GGAAAAGTTCACTGAAGGGTTCATA | Antisense | 2935 |
| 229598_at | 796 | 1161 | 846 | TTGGATACCTTCAAGGGATCAGAAA | Antisense | 2936 |
| 229598_at | 394 | 1017 | 899 | TCATCATTACTCTCTACATTATTAT | Antisense | 2937 |
| 229598_at | 448 | 1109 | 983 | TTTTAACTTGTCTGCATGTTTGAAA | Antisense | 2938 |
| 229623_at | 1098 | 299 | 68 | CAACCCAAAGTCTGAATTTCTTGAG | Antisense | 2939 |
| 229623_at | 173 | 867 | 257 | GGAAGATGGCTGTTTCATGTGAGAC | Antisense | 2940 |
| 229623_at | 764 | 665 | 312 | GATGGGGGAGTGAGAATCCCTGTAA | Antisense | 2941 |
| 229623_at | 677 | 1003 | 374 | TCTCCTGTCAAAAAGGCGTCTTACT | Antisense | 2942 |
| 229623_at | 364 | 887 | 388 | GGCGTCTTACTGTAAATAGTCCAAG | Antisense | 2943 |
| 229623_at | 506 | 27 | 403 | ATAGTCCAAGGTGACATTTGTTAGT | Antisense | 2944 |
| 229623_at | 858 | 709 | 422 | GTTAGTTTTGAATACTGCTTTTGGG | Antisense | 2945 |
| 229623_at | 571 | 1109 | 505 | TTTTCTTTTATTCTACACAATCCAA | Antisense | 2946 |
| 229623_at | 660 | 817 | 544 | GGTGATGCAATTGTACACTTTCTTA | Antisense | 2947 |
| 229623_at | 1051 | 283 | 580 | AATACCACATTTTAGCAGAACCAAG | Antisense | 2948 |
| 229623_at | 607 | 947 | 612 | TGACATTATTCATTGGGATCTGGCC | Antisense | 2949 |
| 230164_at | 440 | 421 | 132 | CCCAGCCTCCAACCTAATTTGAGTT | Antisense | 2950 |
| 230164_at | 900 | 1109 | 169 | TTTTCATCACCTTACATGTGTTAAC | Antisense | 2951 |
| 230164_at | 1138 | 117 | 237 | AGACCGTGTTTACATAATCTCTTCT | Antisense | 2952 |
| 230164_at | 85 | 1033 | 278 | TACTTAACCTTTGCATTGGTCCCTT | Antisense | 2953 |
| 230164_at | 867 | 1159 | 293 | TTGGTCCCTTTCAGGCTTGAACAAC | Antisense | 2954 |
| 230164_at | 516 | 723 | 325 | GTATCTGCATCGTGGGGTTGCTGGT | Antisense | 2955 |
| 230164_at | 125 | 659 | 385 | GAGGATATCCCTGTATTAGGCCATT | Antisense | 2956 |
| 230164_at | 738 | 231 | 437 | AACATACAAGACCTGTGGCTACTGG | Antisense | 2957 |
| 230164_at | 329 | 645 | 508 | GAGATTGTTTAGTGGGCCCATGGCT | Antisense | 2958 |
| 230164_at | 96 | 783 | 519 | GTGGGCCCATGGCTAGGTGGGGTAT | Antisense | 2959 |
| 230164_at | 383 | 425 | 566 | CCCTTGGCACAGATCTTTGAGAATC | Antisense | 2960 |
| 230330_at | 197 | 793 | 51 | GGGCCTCTCACAACATGGCAGTTAT | Antisense | 2961 |
| 230330_at | 892 | 1085 | 73 | TATTTGTCCCCAGAGCAAGCAACAC | Antisense | 2962 |
| 230330_at | 813 | 633 | 85 | GAGCAAGCAACACCGGAGGGCAAGG | Antisense | 2963 |
| 230330_at | 47 | 669 | 120 | GATGTTTTTTGTAACCTAGCCTCTG | Antisense | 2964 |
| 230330_at | 288 | 155 | 133 | ACCTAGCCTCTGAAAGTGTCATACC | Antisense | 2965 |
| 230330_at | 440 | 291 | 158 | AATTCTGTATTTTGTTGGTCACACA | Antisense | 2966 |
| 230330_at | 590 | 743 | 175 | GTCACACAGACCAAGTCAACTACAA | Antisense | 2967 |
| 230330_at | 454 | 1027 | 190 | TCAACTACAACGTGGGAGACTCCTA | Antisense | 2968 |
| 230330_at | 231 | 803 | 203 | GGGAGACTCCTACACAAGGCATGAA | Antisense | 2969 |
| 230330_at | 225 | 817 | 236 | GGTGGGCATTTTTAAGTGTCATCTG | Antisense | 2970 |
| 230330_at | 243 | 867 | 260 | GGAAGGAGGCTGTCACAACCTGGAA | Antisense | 2971 |
| 230521_at | 220 | 891 | 327 | GGCCTCCTCCCAAGGAGAACATGAG | Antisense | 2972 |
| 230521_at | 732 | 849 | 340 | GGAGAACATGAGACCACTTCATATC | Antisense | 2973 |
| 230521_at | 27 | 625 | 351 | GACCACTTCATATCTGGAACTCTAG | Antisense | 2974 |
| 230521_at | 603 | 863 | 366 | GGAACTCTAGAAACACTGGCTGCAG | Antisense | 2975 |
| 230521_at | 252 | 1067 | 373 | TAGAAACACTGGCTGCAGCAATCAA | Antisense | 2976 |
| 230521_at | 237 | 135 | 429 | AGCATGGACACTGCTAGCTGGGTTC | Antisense | 2977 |
| 230521_at | 671 | 497 | 480 | GCTGGGTGACCTCGAGCAAGTGACT | Antisense | 2978 |
| 230521_at | 643 | 621 | 487 | GACCTCGAGCAAGTGACTACTCCTC | Antisense | 2979 |
| 230521_at | 714 | 769 | 499 | GTGACTACTCCTCACTGGATTGTAG | Antisense | 2980 |
| 230521_at | 811 | 747 | 541 | GTCATTATTTACAGTTCACCACACG | Antisense | 2981 |
| 230521_at | 608 | 987 | 584 | TCCCTGGATCCTCAAAATAACCTCA | Antisense | 2982 |
| 230760_at | 812 | 221 | 60 | AAAACGTGACACTTTACATATTTCA | Antisense | 2983 |
| 230760_at | 1006 | 769 | 65 | GTGACACTTTACATATTTCATATTT | Antisense | 2984 |
| 230760_at | 645 | 1085 | 85 | TATTTTTCATATAGTCTGGAGGTAT | Antisense | 2985 |
| 230760_at | 529 | 97 | 97 | AGTCTGGAGGTATACACAGTTGTTT | Antisense | 2986 |
| 230760_at | 802 | 83 | 153 | AGGTAATTAAGCATTTGGTCAAGTA | Antisense | 2987 |
| 230760_at | 985 | 291 | 210 | AATTGTGCTTAATGTTATATACTGT | Antisense | 2988 |
| 230760_at | 131 | 1057 | 229 | TACTGTTATTATTTGCAGGCTGGTT | Antisense | 2989 |
| 230760_at | 864 | 1085 | 238 | TATTTGCAGGCTGGTTTTAATTCTT | Antisense | 2990 |
| 230760_at | 798 | 13 | 367 | ATTAGATATTGTATGTCCCTTCCTC | Antisense | 2991 |
| 230760_at | 12 | 757 | 381 | GTCCCTTCCTCTGTACAAATTGTAA | Antisense | 2992 |
| 230760_at | 937 | 1071 | 436 | TAGGAAGTGTGATACTATTGTAAAT | Antisense | 2993 |
| 230968_at | 484 | 775 | 119 | GTGATTAAACATTCTAGACCAGGCT | Antisense | 2994 |
| 230968_at | 202 | 273 | 195 | AATGTCTTTTCTATTGTGGTCTGAT | Antisense | 2995 |
| 230968_at | 129 | 787 | 210 | GTGGTCTGATATCCGTTTCTGTAAT | Antisense | 2996 |
| 230968_at | 1011 | 107 | 236 | AGATCAGTTTGTTGTCCTCTGTGCA | Antisense | 2997 |
| 230968_at | 955 | 981 | 250 | TCCTCTGTGCACCAGTGGTTTTGCC | Antisense | 2998 |
| 230968_at | 223 | 1035 | 277 | TAATTTTTTTTGGCTAGCATCACCA | Antisense | 2999 |
| 230968_at | 813 | 135 | 292 | AGCATCACCAAGATCTGTCATCCAG | Antisense | 3000 |
| 230968_at | 569 | 3 | 358 | ATTGTGCTGCCAGTGGTATTTTCCC | Antisense | 3001 |
| 230968_at | 1043 | 231 | 441 | AACATGCTCATGGAATGTCCACCTT | Antisense | 3002 |
| 230968_at | 655 | 933 | 518 | TGTTGGTGTTGTTAATGTCATGACT | Antisense | 3003 |
| 230968_at | 769 | 689 | 597 | GATTTTCCTCTAGTCCTATGTGAAT | Antisense | 3004 |
| 231084_at | 24 | 29 | 32 | ATAGCAAATTATGCCCTAAGCTGCA | Antisense | 3005 |
| 231084_at | 968 | 487 | 44 | GCCCTAAGCTGCAATCTACGAGAAG | Antisense | 3006 |
| 231084_at | 465 | 567 | 65 | GAAGAGTTGGTAGCTGTCTCAGAGA | Antisense | 3007 |
| 231084_at | 148 | 253 | 93 | AAGACATCTGTAATGCAATGGGGTC | Antisense | 3008 |
| 231084_at | 3 | 547 | 107 | GCAATGGGGTCTAAACTGACTTGTG | Antisense | 3009 |
| 231084_at | 775 | 581 | 206 | GAACAGATTTCAATACTACAGACTG | Antisense | 3010 |
| 231084_at | 559 | 1143 | 258 | TTCCTGCTCTTGTTCAAATGTAAAA | Antisense | 3011 |
| 231084_at | 690 | 217 | 295 | AAACACAAGGCCAAATCAATCATTT | Antisense | 3012 |
| 231084_at | 723 | 597 | 369 | GAAATTGAAGTAGCATGTCCTCTGA | Antisense | 3013 |
| 231084_at | 1099 | 523 | 381 | GCATGTCCTCTGAGTTGCATGTTTT | Antisense | 3014 |
| 231084_at | 546 | 1051 | 441 | TACAAATTTTTGTCCTGCATGAATA | Antisense | 3015 |
| 231094_s_at | 218 | 885 | 217 | GGCTGAGGAGGCTGGTCTGAACATC | Antisense | 3016 |
| 231094_s_at | 155 | 1001 | 232 | TCTGAACATCACTCACATTTGCCTC | Antisense | 3017 |
| 231094_s_at | 979 | 443 | 253 | CCTCCCTCCAGATAGCAGTGAAGCC | Antisense | 3018 |
| 231094_s_at | 17 | 45 | 341 | ATCTCTGAGAACTTGTTTAGCAACA | Antisense | 3019 |
| 231094_s_at | 438 | 543 | 360 | GCAACAAAGTCCTCAATGCCTTGAA | Antisense | 3020 |
| 231094_s_at | 719 | 247 | 428 | AAGCTGGTGCGAGGGGATGCCCATG | Antisense | 3021 |
| 231094_s_at | 654 | 811 | 440 | GGGGATGCCCATGAATGTTTTGTTT | Antisense | 3022 |
| 231094_s_at | 557 | 469 | 473 | GCCAAAGCTGTAATTGAACTTCTTG | Antisense | 3023 |
| 231094_s_at | 339 | 343 | 551 | CATGGGTCTTTGGAAGCTGCTCTAC | Antisense | 3024 |
| 231094_s_at | 358 | 393 | 567 | CTGCTCTACAATGCCTGTTCCAGAG | Antisense | 3025 |
| 231094_s_at | 882 | 861 | 621 | GGAAAACACGCCAGCTTCAAAGCAA | Antisense | 3026 |
| 231229_at | 843 | 533 | 31 | GCACGTCCAAGGTGATCCTGAGGGC | Antisense | 3027 |
| 231229_at | 969 | 317 | 96 | CACCCTGAAGAAGGCTGTTTCCACC | Antisense | 3028 |
| 231229_at | 999 | 605 | 130 | GACATGGCCCGAAATGCCTATCACT | Antisense | 3029 |
| 231229_at | 632 | 477 | 145 | GCCTATCACTTCAAGCGTGTGCTCA | Antisense | 3030 |
| 231229_at | 105 | 389 | 247 | CTCCAAGTCCAAGCTCAAGGTCAAG | Antisense | 3031 |
| 231229_at | 658 | 603 | 315 | GACAGCACAGGTCACTACTGGGCTC | Antisense | 3032 |
| 231229_at | 453 | 1057 | 330 | TACTGGGCTCCAAACAGGGGCACAA | Antisense | 3033 |
| 231229_at | 908 | 293 | 352 | CAAGCGGCTTATCAAGGGGGTTCGA | Antisense | 3034 |
| 231229_at | 961 | 803 | 369 | GGGTTCGAAGGGTGGCCAAGTGCCA | Antisense | 3035 |
| 231229_at | 847 | 59 | 403 | ATGAGGCAGGCCAGGCAAGCAGTCA | Antisense | 3036 |
| 231229_at | 1130 | 419 | 441 | CGCCATTGGCTCAGTGCAGTGGGAA | Antisense | 3037 |
| 231592_at | 400 | 777 | 257 | GTGTTTTTGTTGCATCTCCAAGTCA | Antisense | 3038 |
| 231592_at | 33 | 851 | 297 | GGAGCAGATATATTTCATTTAGCAT | Antisense | 3039 |
| 231592_at | 112 | 993 | 379 | TCCCACTCTACAACGCATGTCAAAG | Antisense | 3040 |
| 231592_at | 1150 | 1069 | 414 | TAGTTTTTCCTTAGTATCGAACATA | Antisense | 3041 |
| 231592_at | 1131 | 45 | 429 | ATCGAACATATCACAGCTACTCAAT | Antisense | 3042 |
| 231592_at | 375 | 21 | 494 | ATTTGTTCCTGTGTTTCCACCATAA | Antisense | 3043 |
| 231592_at | 773 | 1113 | 578 | TTATTGCTCATATGTCTTCCTGTCT | Antisense | 3044 |
| 231592_at | 802 | 105 | 638 | AGTTGTTACATCTTGAACCATTTAA | Antisense | 3045 |
| 231592_at | 1019 | 213 | 704 | AAACCCTGAGCCCTTTTATCAGTCT | Antisense | 3046 |
| 231592_at | 604 | 1113 | 719 | TTATCAGTCTCCTGGCTTTAAACTA | Antisense | 3047 |
| 231592_at | 1073 | 773 | 757 | GTGATTTGGGGGATTCCTGAAACTA | Antisense | 3048 |
| 231715_s_at | 169 | 295 | 960 | CAATGCAGTTGAGGCCTCCTGTATC | Antisense | 3049 |
| 231715_s_at | 758 | 479 | 973 | GCCTCCTGTATCCGAACACGAGAGC | Antisense | 3050 |
| 231715_s_at | 347 | 229 | 987 | AACACGAGAGCTACAGTCCATGGCC | Antisense | 3051 |
| 231715_s_at | 1125 | 373 | 997 | CTACAGTCCATGGCCGACCAAGAAA | Antisense | 3052 |
| 231715_s_at | 437 | 415 | 1011 | CGACCAAGAAAAGATCTCCCCAGCT | Antisense | 3053 |
| 231715_s_at | 474 | 389 | 1034 | CTGCCCTTAAGAAGACCCTCTTAGA | Antisense | 3054 |
| 231715_s_at | 206 | 187 | 1058 | ACAGAGTGAAGCTGGAATCCCCCAC | Antisense | 3055 |
| 231715_s_at | 136 | 561 | 1149 | GAAGGACTAAGGCAGCATCTGTCCC | Antisense | 3056 |
| 231715_s_at | 312 | 867 | 1282 | GGAAGTTTTCAGGTCCTTGTGATAA | Antisense | 3057 |
| 231715_s_at | 759 | 979 | 1295 | TCCTTGTGATAAAACCTCCTTAAAT | Antisense | 3058 |
| 231715_s_at | 711 | 999 | 1319 | TCTGTTCAGACCAAGCAATGCGAGC | Antisense | 3059 |
| 231890_at | 206 | 355 | 3223 | CATTGTGCCATTCTAGACTTACCTC | Antisense | 3060 |
| 231890_at | 61 | 283 | 3289 | AATAATCTAGCGTCTCTAACCAGTC | Antisense | 3061 |
| 231890_at | 962 | 491 | 3298 | GCGTCTCTAACCAGTCAAACATTTT | Antisense | 3062 |
| 231890_at | 39 | 1121 | 3392 | TTACCTTTAAACCTGCTGCTATGGT | Antisense | 3063 |
| 231890_at | 1140 | 835 | 3519 | GGATTAACATCTTTCTCTTGACACT | Antisense | 3064 |
| 231890_at | 670 | 191 | 3539 | ACACTGAGACTGGGTTCTCCTGGGA | Antisense | 3065 |
| 231890_at | 1152 | 379 | 3608 | CTCTTCTATTTTGCGCTTTTTGTTT | Antisense | 3066 |
| 231890_at | 729 | 361 | 3649 | CTTCTGTTTCTCTACGATGTTTTGA | Antisense | 3067 |
| 231890_at | 700 | 59 | 3680 | ATGAGGCAGTCATGAGAACCCACCA | Antisense | 3068 |
| 231890_at | 412 | 345 | 3690 | CATGAGAACCCACCAGATACAGCTG | Antisense | 3069 |
| 231890_at | 947 | 1051 | 3707 | TACAGCTGCCTGATCCTGAATTTCC | Antisense | 3070 |
| 231929_at | 1064 | 879 | 2289 | GGCTCAAATACCATACCTCAGAAAA | Antisense | 3071 |
| 231929_at | 779 | 587 | 2336 | GAAACAGTCTTTGCAGCTGTGTGAC | Antisense | 3072 |
| 231929_at | 711 | 533 | 2348 | GCAGCTGTGTGACAAGTCACTCTAC | Antisense | 3073 |
| 231929_at | 1117 | 195 | 2359 | ACAAGTCACTCTACTACATACTGAT | Antisense | 3074 |
| 231929_at | 1014 | 165 | 2378 | ACTGATTTGGAGACCTCCGCTAAAT | Antisense | 3075 |
| 231929_at | 912 | 621 | 2389 | GACCTCCGCTAAATAGTTTTATCAC | Antisense | 3076 |
| 231929_at | 855 | 167 | 2412 | ACTGCAGACTAAAATGTGGGACTTG | Antisense | 3077 |
| 231929_at | 472 | 205 | 2606 | AAAGGTTTGTACTGTCTGAGTGCAC | Antisense | 3078 |
| 231929_at | 986 | 753 | 2619 | GTCTGAGTGCACAGCTACTGGAATA | Antisense | 3079 |
| 231929_at | 1101 | 1117 | 2647 | TTAGGGAATCTCAGGAACAAGCATA | Antisense | 3080 |
| 231929_at | 63 | 741 | 2705 | GTAAGTGCAGTTTTTAATTCTGTAT | Antisense | 3081 |
| 232114_at | 603 | 577 | 5119 | GAACTGCAGTGGAATCGTCCAACTA | Antisense | 3082 |
| 232114_at | 1108 | 5 | 5147 | ATTGCCAGTTTTGGTTCTCCTGAAC | Antisense | 3083 |
| 232114_at | 918 | 965 | 5164 | TCCTGAACCTTATGCCACCTTAAGG | Antisense | 3084 |
| 232114_at | 269 | 51 | 5199 | ATCCAGTAGCTGGCTTGAGATTCCA | Antisense | 3085 |
| 232114_at | 462 | 879 | 5210 | GGCTTGAGATTCCAGTGCTCACACT | Antisense | 3086 |
| 232114_at | 907 | 905 | 5240 | TGGTTTCCAGAGAATCTGGCCCCAA | Antisense | 3087 |
| 232114_at | 300 | 555 | 5251 | GAATCTGGCCCCAAAGTCCAGAAGG | Antisense | 3088 |
| 232114_at | 512 | 739 | 5319 | GTAAGTATTTGCTCTTTTGAATTTA | Antisense | 3089 |
| 232114_at | 380 | 779 | 5357 | GTGTCAGTCTTGGTTGTGTATTGCA | Antisense | 3090 |
| 232114_at | 1018 | 725 | 5474 | GTATGTGGTAATTTGCTCCTATTAG | Antisense | 3091 |
| 232114_at | 363 | 23 | 5629 | ATATTTTCCCATGTTCTCTGTTATT | Antisense | 3092 |
| 232204_at | 1065 | 189 | 1804 | ACAGCCTGCAAGCGATATCTGGCAT | Antisense | 3093 |
| 232204_at | 6 | 1081 | 1819 | TATCTGGCATGATTGTTCCTCCTAT | Antisense | 3094 |
| 232204_at | 843 | 701 | 1833 | GTTCCTCCTATGTGAAAGAATTGCC | Antisense | 3095 |
| 232204_at | 146 | 641 | 1898 | GAGAGTAATCTGATACAAGTCCCAG | Antisense | 3096 |
| 232204_at | 370 | 1045 | 1911 | TACAAGTCCCAGAGTGGAACTTTTA | Antisense | 3097 |
| 232204_at | 895 | 223 | 1980 | AACAAACAAAATCACCGACCTTGCA | Antisense | 3098 |
| 232204_at | 397 | 319 | 1992 | CACCGACCTTGCAAATACTGAAATT | Antisense | 3099 |
| 232204_at | 36 | 1161 | 2015 | TTGGAAGAGGGATCTGCAAGTGCAG | Antisense | 3100 |
| 232204_at | 193 | 93 | 2033 | AGTGCAGGGTGTTGGTTAAAGTTGT | Antisense | 3101 |
| 232204_at | 1064 | 515 | 2140 | GCTTAATTCTGCAATCATTTGTCTT | Antisense | 3102 |
| 232204_at | 144 | 1047 | 2175 | TAAAGCTCTATACAAGGGACACTAT | Antisense | 3103 |
| 232618_at | 394 | 127 | 260 | AGAAGGCCGTGACTTTATAACAAAG | Antisense | 3104 |
| 232618_at | 770 | 645 | 290 | GAGTTGGGTGAGCCCAGTGCACTGC | Antisense | 3105 |
| 232618_at | 820 | 335 | 304 | CAGTGCACTGCACACAAGAGTCAAG | Antisense | 3106 |
| 232618_at | 454 | 387 | 358 | CTCCGTCTGGTCAGCAAGCTTTACA | Antisense | 3107 |
| 232618_at | 334 | 499 | 497 | GCTGATCTTGGAAATTCACCTCTTC | Antisense | 3108 |
| 232618_at | 44 | 611 | 576 | GACTATGGAAACATTCTCAACAGGG | Antisense | 3109 |
| 232618_at | 1114 | 1029 | 592 | TCAACAGGGAAACCCTACTAGACTT | Antisense | 3110 |
| 232618_at | 488 | 17 | 711 | ATTTATGGGAATTATCACTGACAGT | Antisense | 3111 |
| 232618_at | 887 | 1081 | 723 | TATCACTGACAGTTTTTGTACACTT | Antisense | 3112 |
| 232618_at | 198 | 777 | 755 | GTGTTAAAGCAGCAACTCCATGTTG | Antisense | 3113 |
| 232618_at | 944 | 543 | 766 | GCAACTCCATGTTGTAAATGCACAA | Antisense | 3114 |
| 233976_at | 677 | 939 | 1736 | TGAGTCTTTTGAACACAGGTATGAA | Antisense | 3115 |
| 233976_at | 225 | 1007 | 1773 | TCTAAACCAATGTTACCCCTACCAT | Antisense | 3116 |
| 233976_at | 757 | 707 | 1784 | GTTACCCCTACCATACAGGTATGTT | Antisense | 3117 |
| 233976_at | 975 | 243 | 1874 | AACCACATTAAATATTCTGTCCATT | Antisense | 3118 |
| 233976_at | 985 | 957 | 1951 | TGCAGCATGCAAGGCAAGATGACAA | Antisense | 3119 |
| 233976_at | 76 | 201 | 2024 | AAATAATTATGACATGCTTTCTGAA | Antisense | 3120 |
| 233976_at | 948 | 261 | 2047 | AAGTGTCCCCAGATAGTTTCATAAA | Antisense | 3121 |
| 233976_at | 127 | 587 | 2093 | GAAAAACCCATAGACATCATGTTCA | Antisense | 3122 |
| 233976_at | 1001 | 935 | 2125 | TGATGACAAGGCATGTTCATAACCC | Antisense | 3123 |
| 233976_at | 725 | 313 | 2168 | CAGCTAAATAACTGCAATACTTTTG | Antisense | 3124 |
| 233976_at | 781 | 647 | 2196 | GAGTTTGGCAGGTTTCGAAGATGTG | Antisense | 3125 |
| 235048_at | 254 | 1021 | 1013 | TCAGTATTTTTATTCGCCTTCTAGA | Antisense | 3126 |
| 235048_at | 746 | 51 | 1045 | ATCCACACATCACCCATTTATATTA | Antisense | 3127 |
| 235048_at | 697 | 879 | 1107 | GGCTTACCTTCTGTCATCAAGTGAT | Antisense | 3128 |
| 235048_at | 142 | 723 | 1133 | GTATCATCCTGGATCGTCATTTCCA | Antisense | 3129 |
| 235048_at | 1087 | 747 | 1148 | GTCATTTCCAAGGAACTAGCCTTTC | Antisense | 3130 |
| 235048_at | 168 | 367 | 1168 | CTTTCTTTTCCTAAGCGTCTGTATG | Antisense | 3131 |
| 235048_at | 946 | 725 | 1188 | GTATGTGTTCTAAAACTTCCAGTAT | Antisense | 3132 |
| 235048_at | 1009 | 403 | 1265 | CTGGAGTACCTATGTTTGTTTTCTT | Antisense | 3133 |
| 235048_at | 358 | 695 | 1341 | GATTGTTTCCTGGTCTGTGTTTTTA | Antisense | 3134 |
| 235048_at | 258 | 1097 | 1372 | TTTCCTTCAGTTTTCCTCATGAAGA | Antisense | 3135 |
| 235048_at | 762 | 37 | 1453 | ATCACATTGGTTGTACTCTGAAGAC | Antisense | 3136 |
| 235092_at | 544 | 449 | 1270 | CCTTTCAAACGTTTACCAGCTAATG | Antisense | 3137 |
| 235092_at | 452 | 593 | 1317 | GAAAGGGGCTGCGAGTGGTAATATA | Antisense | 3138 |
| 235092_at | 1139 | 251 | 1367 | AAGACCTGTAGGCTATGTGTCTTCT | Antisense | 3139 |
| 235092_at | 639 | 605 | 1419 | GACAGGTTGTGTTAGTGAATTTTAA | Antisense | 3140 |
| 235092_at | 597 | 551 | 1544 | GAATAGCACCTGATTAATTAAGAGT | Antisense | 3141 |
| 235092_at | 65 | 593 | 1580 | GAAAGTTTATTAAGGCGTGCATGGC | Antisense | 3142 |
| 235092_at | 61 | 959 | 1597 | TGCATGGCCTTGGGATAACAATGAA | Antisense | 3143 |
| 235092_at | 791 | 949 | 1618 | TGAAACCATTTGTCTACTGGCATCT | Antisense | 3144 |
| 235092_at | 934 | 401 | 1641 | CTGTCTGCTGGTTTCTTGACACCAG | Antisense | 3145 |
| 235092_at | 202 | 1155 | 1732 | TTGTTCTAAGGAATGCAGTCTTTCA | Antisense | 3146 |
| 235092_at | 351 | 959 | 1745 | TGCAGTCTTTCAGTCATTATCTTTT | Antisense | 3147 |
| 235310_at | 224 | 413 | 1348 | CGATCCTTGGAGATCCCGTAATCCC | Antisense | 3148 |
| 235310_at | 524 | 1091 | 1441 | TTTGGAGCCTGATTTCCTACTGACT | Antisense | 3149 |
| 235310_at | 1002 | 1095 | 1453 | TTTCCTACTGACTTCCAATTTAGTG | Antisense | 3150 |
| 235310_at | 251 | 963 | 1476 | TGCTCCCCCAGTATGCTAAATAGAA | Antisense | 3151 |
| 235310_at | 1065 | 283 | 1494 | AATAGAAAGCCCTCTGCAATATATT | Antisense | 3152 |
| 235310_at | 1119 | 689 | 1636 | GATTATTTACTTTCTCTTATCTTTT | Antisense | 3153 |
| 235310_at | 576 | 1113 | 1652 | TTATCTTTTCCTTAGTGTTCCTCAA | Antisense | 3154 |
| 235310_at | 230 | 199 | 1675 | AAATTATATCTATCCTCTAAACCAG | Antisense | 3155 |
| 235310_at | 107 | 89 | 1698 | AGGGATCAGCAAACTATAACCCCCA | Antisense | 3156 |
| 235310_at | 59 | 173 | 1781 | ACTCATTTGTTTACCTACTATCTAT | Antisense | 3157 |
| 235310_at | 1018 | 947 | 1838 | TGACATGGACCATAGGCCCTAAAGA | Antisense | 3158 |
| 235569_at | 411 | 479 | 417 | GCCTCTAGGGTGATGGACCAGCCCC | Antisense | 3159 |
| 235569_at | 401 | 927 | 452 | TTGACTCAACTACAGGGGCCTGGGA | Antisense | 3160 |
| 235569_at | 334 | 269 | 553 | AAGGCCGCCAGGAGTGTTTTACATC | Antisense | 3161 |
| 235569_at | 1104 | 1073 | 622 | TAGGAGCCAGGTGATCAGGCCTCGG | Antisense | 3162 |
| 235569_at | 915 | 49 | 695 | ATGCTCCAGACCCTGGGGCAAGGTA | Antisense | 3163 |
| 235569_at | 147 | 547 | 712 | GCAAGGTAGGCCAGGGGCTTCTGAC | Antisense | 3164 |
| 235569_at | 657 | 879 | 727 | GGCTTCTGACCTGTGCAGGTGAGAG | Antisense | 3165 |
| 235569_at | 645 | 791 | 753 | GGGCCATACCCAGGAAAGACCATTC | Antisense | 3166 |
| 235569_at | 476 | 401 | 787 | CTGTCCCTGTCTCCTTAGAATGGAA | Antisense | 3167 |
| 235569_at | 659 | 135 | 811 | AGCTTTTTGAGGGCAGGTCCTTGTC | Antisense | 3168 |
| 235569_at | 179 | 339 | 824 | CAGGTCCTTGTCTTTGTACGTTCTG | Antisense | 3169 |
| 235885_at | 734 | 203 | 217 | AAATGTATATATATCCTAGTCCCCT | Antisense | 3170 |
| 235885_at | 973 | 423 | 238 | CCCTAACCAAATCCTGACCTATTGG | Antisense | 3171 |
| 235885_at | 521 | 441 | 250 | CCTGACCTATTGGGATACTTATAAA | Antisense | 3172 |
| 235885_at | 457 | 589 | 357 | GAAACTGTATTTGATTGGACTTAAT | Antisense | 3173 |
| 235885_at | 923 | 637 | 463 | GAGACTTACTTTTATGACATTCTAA | Antisense | 3174 |
| 235885_at | 448 | 195 | 547 | ACAACTTCAATAATACCATCACTGA | Antisense | 3175 |
| 235885_at | 392 | 281 | 555 | AATAATACCATCACTGACACTTACC | Antisense | 3176 |
| 235885_at | 1000 | 599 | 570 | GACACTTACCTTTATTAATTAGCTT | Antisense | 3177 |
| 235885_at | 799 | 25 | 657 | ATATGATTACAAAGTTGCACAGCAT | Antisense | 3178 |
| 235885_at | 929 | 699 | 670 | GTTGCACAGCATAACTACTGAGAGG | Antisense | 3179 |
| 235885_at | 947 | 693 | 700 | GATTGATCTGTTTGTAATTACTTGT | Antisense | 3180 |
| 236694_at | 897 | 13 | 39 | ATTAGAGCTATATCATTTTACTCTG | Antisense | 3181 |
| 236694_at | 784 | 15 | 53 | ATTTTACTCTGAATGTCTTAACATA | Antisense | 3182 |
| 236694_at | 763 | 695 | 99 | GATTGGATTGATTTCAGCCTTCTTC | Antisense | 3183 |
| 236694_at | 199 | 695 | 104 | GATTGATTTCAGCCTTCTTCTGGTA | Antisense | 3184 |
| 236694_at | 44 | 1129 | 111 | TTCAGCCTTCTTCTGGTACTTTTTA | Antisense | 3185 |
| 236694_at | 649 | 1079 | 236 | TATGCTGATCTTGTGAGTATTTATT | Antisense | 3186 |
| 236694_at | 662 | 33 | 284 | ATAAAATCTATGTGTGCTTGATAAG | Antisense | 3187 |
| 236694_at | 73 | 195 | 372 | ACAATCTCCTTTCTTTAGTACTTAT | Antisense | 3188 |
| 236694_at | 804 | 1003 | 376 | TCTCCTTTCTTTAGTACTTATGATT | Antisense | 3189 |
| 236694_at | 1152 | 733 | 389 | GTACTTATGATTGGTGTGTGTGACT | Antisense | 3190 |
| 236694_at | 572 | 817 | 401 | GGTGTGTGTGACTTCATCTTATGAA | Antisense | 3191 |
| 236707_at | 1055 | 977 | 444 | TCCAGGGTCTCTACTGTTCTCCTGG | Antisense | 3192 |
| 236707_at | 521 | 365 | 470 | CTTTTCTCATGCTGGCTACTTCAGG | Antisense | 3193 |
| 236707_at | 1043 | 189 | 510 | ACACCTGTAGGTCTCATATTTTCAA | Antisense | 3194 |
| 236707_at | 452 | 841 | 557 | GGATGATATTACCCGTGTCTGTCCC | Antisense | 3195 |
| 236707_at | 600 | 923 | 572 | TGTCTGTCCCTTTTTATCAGGAAAA | Antisense | 3196 |
| 236707_at | 58 | 391 | 606 | CTCCAGAAACTCCCCTATGAGACCG | Antisense | 3197 |
| 236707_at | 273 | 837 | 681 | GGATTTATTGCTTGGGGTTCTGTTA | Antisense | 3198 |
| 236707_at | 762 | 101 | 801 | AGTATATGCACTGTATCACTCCCTC | Antisense | 3199 |
| 236707_at | 464 | 975 | 847 | TCCACTAGACCTCACATCCAGGTAT | Antisense | 3200 |
| 236707_at | 371 | 517 | 874 | GCTTCTATGCACAGAGACTTACCTC | Antisense | 3201 |
| 236707_at | 34 | 1061 | 893 | TACCTCTTACCTATTCAACTTTAAT | Antisense | 3202 |
| 238106_at | 899 | 799 | 187 | GGGACAGACACTTGTCATTGCCAAG | Antisense | 3203 |
| 238106_at | 867 | 219 | 265 | AAAAATAGCACAGTCATTCCTTTGG | Antisense | 3204 |
| 238106_at | 938 | 409 | 285 | TTTGGACTAAGTCAACTCCTAGGCC | Antisense | 3205 |
| 238106_at | 1145 | 479 | 307 | GCCTCCTGAGAGCACTGTAGCTGTG | Antisense | 3206 |
| 238106_at | 706 | 741 | 377 | GTCACAGGGTACTTTCCTATATACC | Antisense | 3207 |
| 238106_at | 1046 | 1083 | 394 | TATATACCAGCGTACACCTCTATCA | Antisense | 3208 |
| 238106_at | 721 | 191 | 466 | ACACTGCCTCATGCACAGATATAGA | Antisense | 3209 |
| 238106_at | 1031 | 365 | 506 | CTTTCTTTCCCCTGCAATGCAGAAA | Antisense | 3210 |
| 238106_at | 1001 | 193 | 546 | ACAACCACACTAAACCAACCAGAAT | Antisense | 3211 |
| 238106_at | 1145 | 831 | 640 | GGTTAATGAGTCCATACTCCTGCCT | Antisense | 3212 |
| 238106_at | 96 | 479 | 661 | GCCTCCAGTCAGAACTGTCCTAAGC | Antisense | 3213 |
| 238693_at | 1056 | 485 | 214 | GCCCTGAAAGCTTTATAGTCCTGGA | Antisense | 3214 |
| 238693_at | 329 | 863 | 323 | GGAACATACACAGATATTTGGCAGA | Antisense | 3215 |
| 238693_at | 1099 | 871 | 342 | GGCAGAAGTCGAGTAAGGAGGGGAA | Antisense | 3216 |
| 238693_at | 956 | 255 | 370 | AAGAGTCCGTGAGTTTCAGTCATTT | Antisense | 3217 |
| 238693_at | 1080 | 715 | 382 | GTTTCAGTCATTTTCACTGCTCTTT | Antisense | 3218 |
| 238693_at | 144 | 925 | 444 | TGTCACTGAAGACATCACAGATACT | Antisense | 3219 |
| 238693_at | 209 | 23 | 469 | ATATTTATCTTTTGGCTTTGTGTAC | Antisense | 3220 |
| 238693_at | 650 | 35 | 660 | ATAAGGCTCATGTTCCAGACCTACG | Antisense | 3221 |
| 238693_at | 727 | 1017 | 667 | TCATGTTCCAGACCTACGATAGCTT | Antisense | 3222 |
| 238693_at | 224 | 685 | 684 | GATAGCTTGTAGTCTATGTTACGTA | Antisense | 3223 |
| 238693_at | 762 | 1077 | 698 | TATGTTACGTATTTCTTTATATCAC | Antisense | 3224 |
| 239146_at | 523 | 933 | 326 | TGATCTCCAAACCCTTATTTTTTCT | Antisense | 3225 |
| 239146_at | 160 | 1147 | 365 | TTCCACTCATGGACGATGCAACCAA | Antisense | 3226 |
| 239146_at | 184 | 545 | 382 | GCAACCAACAGATGCAATCTCTGAG | Antisense | 3227 |
| 239146_at | 1146 | 669 | 409 | GATGAAAATTGGGACCTCTTATTAT | Antisense | 3228 |
| 239146_at | 986 | 943 | 439 | TGACCTAGCTGGACTCAGGAAACCA | Antisense | 3229 |
| 239146_at | 933 | 1109 | 554 | TTTTCTGCTCTGGTGTATAACAAAC | Antisense | 3230 |
| 239146_at | 432 | 303 | 574 | CAAACAGGTCAAAATTTCCCATCTT | Antisense | 3231 |
| 239146_at | 656 | 453 | 592 | CCATCTTTCCTCCTGATAGTAGTTG | Antisense | 3232 |
| 239146_at | 156 | 1149 | 614 | TTGAATCCTACCTTGCATACTTAAT | Antisense | 3233 |
| 239146_at | 500 | 303 | 676 | CAAAACACACCACCATTTCATTAGG | Antisense | 3234 |
| 239146_at | 635 | 1149 | 745 | TTGCTTTTTCTTAACCCACTATATA | Antisense | 3235 |
| 239486_at | 540 | 1043 | 156 | TAAAGTTGGGCAGTAATCTTAATTA | Antisense | 3236 |
| 239486_at | 1024 | 929 | 224 | TGTTTTACAACTAGAACCTGCCCTA | Antisense | 3237 |
| 239486_at | 21 | 1067 | 235 | TAGAACCTGCCCTAAATGTTGAATA | Antisense | 3238 |
| 239486_at | 826 | 273 | 249 | AATGTTGAATATCTTCCTAGCAAGA | Antisense | 3239 |
| 239486_at | 754 | 359 | 261 | CTTCCTAGCAAGAAACAGTCTGTCA | Antisense | 3240 |
| 239486_at | 374 | 335 | 276 | CAGTCTGTCATTTTACTTACACGAT | Antisense | 3241 |
| 239486_at | 1134 | 171 | 290 | ACTTACACGATGTCTAACCAAACCA | Antisense | 3242 |
| 239486_at | 871 | 745 | 301 | GTCTAACCAAACCATAACTTTACAT | Antisense | 3243 |
| 239486_at | 37 | 1053 | 321 | TACATAAACTAGTCGTTTCGGTCAA | Antisense | 3244 |
| 239486_at | 933 | 619 | 467 | GACCCCTGAGTACCATTAATATTCC | Antisense | 3245 |
| 239486_at | 874 | 631 | 543 | GAGCAGCCATTATGAAATCTCACAA | Antisense | 3246 |
| 239499_at | 991 | 763 | 34 | GTGCTGGCTGGGACCGGAAGAACTC | Antisense | 3247 |
| 239499_at | 246 | 157 | 46 | ACCGGAAGAACTCCTGCTTGGTGGA | Antisense | 3248 |
| 239499_at | 775 | 359 | 113 | CTTCCGGCCTGCAGAGAGCCGCAAG | Antisense | 3249 |
| 239499_at | 729 | 567 | 137 | GAAGAGCGCCAAGGGCATGTACTCC | Antisense | 3250 |
| 239499_at | 548 | 1083 | 171 | TATTACTATCCCAACCGGGCAGGCA | Antisense | 3251 |
| 239499_at | 929 | 791 | 241 | GGGCCATGACACCTGATCATTGGAT | Antisense | 3252 |
| 239499_at | 235 | 1031 | 265 | TCAAGAGGGGCACTGCTCTACTCAT | Antisense | 3253 |
| 239499_at | 742 | 381 | 280 | CTCTACTCATGAGCCTGGACAGCTG | Antisense | 3254 |
| 239499_at | 704 | 317 | 370 | CACCTAGGACTGAGGCCGGACCTCA | Antisense | 3255 |
| 239499_at | 120 | 377 | 395 | CTCAGACTTTGACCTTGGCCGAATT | Antisense | 3256 |
| 239499_at | 1008 | 667 | 425 | GATGTGGCCCTGGAGATACCTAGTT | Antisense | 3257 |
| 239726_at | 910 | 113 | 131 | AGAGTCACTACCCGAGTTATTCGCC | Antisense | 3258 |
| 239726_at | 569 | 711 | 146 | GTTATTCGCCGGCGTTTGATTATAA | Antisense | 3259 |
| 239726_at | 310 | 207 | 169 | AAAGGTATCCTGTAGCTGGATCATT | Antisense | 3260 |
| 239726_at | 494 | 837 | 186 | GGATCATTATGTGGCATTGCTCTAC | Antisense | 3261 |
| 239726_at | 263 | 1119 | 213 | TTAGAAACTCCTTCCTTGTGGTTGT | Antisense | 3262 |
| 239726_at | 994 | 1155 | 234 | TTGTAGAAACTCCTTCCTTGTGGTT | Antisense | 3263 |
| 239726_at | 303 | 17 | 300 | ATTTTTTTGCACCCTTGAAAGCTTG | Antisense | 3264 |
| 239726_at | 696 | 525 | 360 | GCATGGTATTCTGTATAGGCCTATT | Antisense | 3265 |
| 239726_at | 557 | 685 | 386 | GATATGTGGCGGTTGTAGTTTTATT | Antisense | 3266 |
| 239726_at | 78 | 751 | 599 | GTCTCAAATTCCTACTGCCTTAAGA | Antisense | 3267 |
| 239726_at | 629 | 243 | 637 | AACCAGTTCTTCATAAGCAATCAGT | Antisense | 3268 |
| 239834_at | 800 | 281 | 15 | AATAACTGCTAAGGTCCTATTTTGA | Antisense | 3269 |
| 239834_at | 940 | 111 | 52 | AGAGGGAGGGATGTTCTTTATTTCT | Antisense | 3270 |
| 239834_at | 273 | 143 | 117 | AGCGATTGTAACAAGACTTCTTGCC | Antisense | 3271 |
| 239834_at | 447 | 609 | 131 | GACTTCTTGCCATGGTTATTTGGAG | Antisense | 3272 |
| 239834_at | 919 | 629 | 153 | GAGCTATTAAGATTAACTGCCTTTT | Antisense | 3273 |
| 239834_at | 172 | 1071 | 209 | TAGTTTGTGGCTACATTCCTTTTTC | Antisense | 3274 |
| 239834_at | 635 | 959 | 249 | TGCTCTCCTTTATGAAAAGTTCAAG | Antisense | 3275 |
| 239834_at | 107 | 341 | 324 | CAGGAAGTAGTTACTGAGCGCCTAC | Antisense | 3276 |
| 239834_at | 400 | 1049 | 376 | TAAATTAATCGTTTTCCACCTGGCA | Antisense | 3277 |
| 239834_at | 736 | 1095 | 388 | TTTCCACCTGGCAAGTAGTCTCATT | Antisense | 3278 |
| 239834_at | 723 | 437 | 394 | CCTGGCAAGTAGTCTCATTGGCAAA | Antisense | 3279 |
| 240013_at | 431 | 357 | 14 | CTTGCGATGCATCTTACTCTGTTTA | Antisense | 3280 |
| 240013_at | 184 | 525 | 52 | GCATGTCTGTTTTTTGACGGCATCT | Antisense | 3281 |
| 240013_at | 1058 | 87 | 150 | AGGGCAATTCCCTTTTCTGATATTG | Antisense | 3282 |
| 240013_at | 438 | 399 | 166 | CTGATATTGCTAGCTGTGTTTCTTG | Antisense | 3283 |
| 240013_at | 648 | 777 | 181 | GTGTTTCTTGGGTTGCCACTATTGC | Antisense | 3284 |
| 240013_at | 178 | 701 | 192 | GTTGCCACTATTGCCACTGAATGAA | Antisense | 3285 |
| 240013_at | 554 | 1133 | 226 | TTCTTCACCTAACCAGGTAATCTGC | Antisense | 3286 |
| 240013_at | 724 | 279 | 244 | AATCTGCAGTTCTCATAATCGCTTT | Antisense | 3287 |
| 240013_at | 605 | 35 | 258 | ATAATCGCTTTGTGCTATTGATGAG | Antisense | 3288 |
| 240013_at | 786 | 529 | 282 | GCAGTCATATGTTTTTCAATGCTAT | Antisense | 3289 |
| 240013_at | 295 | 61 | 337 | ATGATGCTTTCGATTGTTATACTAA | Antisense | 3290 |
| 241671_x_at | 579 | 1 | 28 | ATTCTGAAGCCAGACCGTCTTTCCT | Antisense | 3291 |
| 241671_x_at | 716 | 993 | 223 | TCGCAGCTGGGCGAGGGGACTTGGA | Antisense | 3292 |
| 241671_x_at | 861 | 403 | 229 | CTGGGCGAGGGGACTTGGAGGACAG | Antisense | 3293 |
| 241671_x_at | 555 | 813 | 237 | GGGGACTTGGAGGACAGGGTGAAGC | Antisense | 3294 |
| 241671_x_at | 958 | 177 | 241 | ACTTGGAGGACAGGGTGAAGCTGCA | Antisense | 3295 |
| 241671_x_at | 834 | 951 | 256 | TGAAGCTGCAGAAGACCTGGGGTGG | Antisense | 3296 |
| 241671_x_at | 1116 | 501 | 260 | GCTGCAGAAGACCTGGGGTGGGATG | Antisense | 3297 |
| 241671_x_at | 425 | 1067 | 287 | TAGAGAGGACGCCAAGGACTGGGGA | Antisense | 3298 |
| 241671_x_at | 19 | 113 | 290 | AGAGGACGCCAAGGACTGGGGAAGG | Antisense | 3299 |
| 241671_x_at | 516 | 865 | 315 | GGAAGTTAGGAATACCTTACATCCA | Antisense | 3300 |
| 241671_x_at | 536 | 75 | 322 | AGGAATACCTTACATCCAATGCCCA | Antisense | 3301 |
| 241956_at | 521 | 395 | 54 | CTGCAAAGCAGTATTCCTCAGCCTT | Antisense | 3302 |
| 241956_at | 413 | 835 | 80 | GGTTGGGTCTTTGATATGTGGTGAT | Antisense | 3303 |
| 241956_at | 209 | 775 | 100 | GTGATATCCTGGGTTTCAACTCTTC | Antisense | 3304 |
| 241956_at | 5 | 683 | 156 | GATACAGTTGTTCTGTGATTTGGGA | Antisense | 3305 |
| 241956_at | 757 | 1117 | 259 | TTAGATCTTGACTTAGGCATATACA | Antisense | 3306 |
| 241956_at | 219 | 261 | 309 | AAGTCTACAATGTTATGATGCTACA | Antisense | 3307 |
| 241956_at | 220 | 241 | 356 | AACGCTAGGTTTTATGGAGACTCAT | Antisense | 3308 |
| 241956_at | 464 | 257 | 392 | AAGATTTTCGTCTTACTATTCAAGT | Antisense | 3309 |
| 241956_at | 602 | 55 | 430 | ATGAAGCTTTATAATTGGTGTGCAA | Antisense | 3310 |
| 241956_at | 22 | 215 | 454 | AAACCAGACATAGAACCTGCCCTTC | Antisense | 3311 |
| 241956_at | 767 | 1139 | 476 | TTCTGGACTCTCTGGTCAAGTGAAG | Antisense | 3312 |
| 242469_at | 474 | 837 | 74 | GGATATTGTAAACACACCTCAGGGA | Antisense | 3313 |
| 242469_at | 251 | 327 | 117 | CAGACAAACAGGCCAATCCGGGGGA | Antisense | 3314 |
| 242469_at | 534 | 591 | 156 | GAAAGCTCTACAATGAGGCAGATTT | Antisense | 3315 |
| 242469_at | 479 | 297 | 199 | CAAGTCCTTCAGCTCAAGCTGCAGC | Antisense | 3316 |
| 242469_at | 718 | 517 | 259 | GCTTCCTGGTTCATGACATTACAGC | Antisense | 3317 |
| 242469_at | 383 | 353 | 275 | CATTACAGCATGGATCGTTTTCAAA | Antisense | 3318 |
| 242469_at | 1096 | 225 | 297 | AAAATTAATACTGCCTCCTGTTTCA | Antisense | 3319 |
| 242469_at | 2 | 1057 | 305 | TACTGCCTCCTGTTTCAGCAGTGGC | Antisense | 3320 |
| 242469_at | 1014 | 1003 | 342 | TCTCCCTGTGGCTCAGAGTATGGGA | Antisense | 3321 |
| 242469_at | 406 | 801 | 363 | GGGAGTGAGTATGCCCTGTAACCCA | Antisense | 3322 |
| 242469_at | 633 | 553 | 421 | GAATCACAGAGCAATACGGGTACCT | Antisense | 3323 |
| 242765_at | 31 | 299 | 41 | CAAGACTACACTTTCTTGCCTTCTT | Antisense | 3324 |
| 242765_at | 627 | 153 | 128 | ACCTTGGAACTCTGTTCTTGCTCTT | Antisense | 3325 |
| 242765_at | 49 | 359 | 144 | CTTGCTCTTCATTCCGCATGTGATA | Antisense | 3326 |
| 242765_at | 397 | 1143 | 155 | TTCCGCATGTGATACTCTGGTCCAA | Antisense | 3327 |
| 242765_at | 246 | 285 | 206 | AATATCGTCTCAGAGGTGCTTCCCT | Antisense | 3328 |
| 242765_at | 502 | 111 | 217 | AGAGGTGCTTCCCTTGAAAACTCGG | Antisense | 3329 |
| 242765_at | 698 | 171 | 236 | ACTCGGTGCTGTTTCCATAGTTACT | Antisense | 3330 |
| 242765_at | 61 | 1071 | 253 | TAGTTACTCTATTTGATCACTCTAA | Antisense | 3331 |
| 242765_at | 1147 | 679 | 267 | GATCACTCTAAGTTTGGTTGTCTTC | Antisense | 3332 |
| 242765_at | 53 | 427 | 359 | CCCTCCTAACGTAAACTCCATGATT | Antisense | 3333 |
| 242765_at | 667 | 237 | 372 | AACTCCATGATTGCCAACACCTGTT | Antisense | 3334 |
| 242874_at | 729 | 529 | 35 | GCAGTTCCAGTCTCACAGAAGTCTG | Antisense | 3335 |
| 242874_at | 832 | 561 | 84 | GAAGGTGGTCTTTTCATTCTACTCA | Antisense | 3336 |
| 242874_at | 183 | 1131 | 96 | TTCATTCTACTCAGGTCTTCTATTG | Antisense | 3337 |
| 242874_at | 1090 | 31 | 138 | ATACATCATGGAGGGCAATCTTCTT | Antisense | 3338 |
| 242874_at | 562 | 797 | 150 | GGGCAATCTTCTTTACTCAGAGGAC | Antisense | 3339 |
| 242874_at | 434 | 779 | 179 | GTGTCAATGTTATTCCCATCCAGAA | Antisense | 3340 |
| 242874_at | 57 | 453 | 194 | CCATCCAGAAACACCTTCCAAGTTG | Antisense | 3341 |
| 242874_at | 269 | 1045 | 230 | TAACAATACACTTTTCCTCATCTCA | Antisense | 3342 |
| 242874_at | 217 | 1097 | 242 | TTTCCTCATCTCACCCAAATTTAGG | Antisense | 3343 |
| 242874_at | 676 | 1159 | 301 | TTGGAATGCCACTTTTACTTTTGGA | Antisense | 3344 |
| 242874_at | 1035 | 871 | 372 | GGCACACTGGTTTACACCTATAATC | Antisense | 3345 |
| 243010_at | 200 | 597 | 608 | GAAATACACCCACTCTCTTGGAATA | Antisense | 3346 |
| 243010_at | 1151 | 57 | 633 | ATGACGTACCACTCAGTTGGACCCT | Antisense | 3347 |
| 243010_at | 856 | 619 | 652 | GACCCTCAAGAGTCACTGCTTTGTC | Antisense | 3348 |
| 243010_at | 506 | 417 | 704 | CGCACGCTTCCATTTGATGCATTTG | Antisense | 3349 |
| 243010_at | 1129 | 63 | 753 | ATGTCATTGTCCTTGAGACCCTACA | Antisense | 3350 |
| 243010_at | 296 | 639 | 767 | GAGACCCTACATGTGCAGTTTGGCT | Antisense | 3351 |
| 243010_at | 825 | 1097 | 840 | TTTCCTGCAGGCTTTTCCATGAGTA | Antisense | 3352 |
| 243010_at | 970 | 579 | 879 | GAACAAATCTGTATGGCTTTTCCCC | Antisense | 3353 |
| 243010_at | 483 | 767 | 943 | GTGAACTTGTCCTAGTATGCTTGCC | Antisense | 3354 |
| 243010_at | 1033 | 357 | 962 | CTTGCCTCACAAACGTTTTAGCCAT | Antisense | 3355 |
| 243010_at | 136 | 365 | 1119 | CTTAGCCTGACAGTGTCCTGTTCTC | Antisense | 3356 |
| 243170_at | 255 | 1017 | 52 | TCATATAATATGTTGCTGCTTTTGG | Antisense | 3357 |
| 243170_at | 560 | 811 | 77 | GGGGGTGTTGGGGAGTACCAGAGAG | Antisense | 3358 |
| 243170_at | 56 | 525 | 106 | GCATGCATCTGCAGTGTACACTGGC | Antisense | 3359 |
| 243170_at | 813 | 541 | 129 | GCAAACAAAAGGTTTGTCATTCCCG | Antisense | 3360 |
| 243170_at | 1069 | 839 | 221 | GGATGCAGGTGTAGTCTTGTGCCAG | Antisense | 3361 |
| 243170_at | 57 | 729 | 231 | GTAGTCTTGTGCCAGTCTGTGACAT | Antisense | 3362 |
| 243170_at | 649 | 769 | 249 | GTGACATAACACCTGAACAATTTCT | Antisense | 3363 |
| 243170_at | 1156 | 581 | 263 | GAACAATTTCTATGGTATCCTACCC | Antisense | 3364 |
| 243170_at | 833 | 1079 | 278 | TATCCTACCCATTTGCTCAGATTCA | Antisense | 3365 |
| 243170_at | 1142 | 507 | 292 | GCTCAGATTCAAACCAGGCCTTAAT | Antisense | 3366 |
| 243170_at | 287 | 47 | 304 | ACCAGGCCTTAATCGATAACAAGCA | Antisense | 3367 |
| 244008_at | 1036 | 1053 | 112 | TACATTTCACATTTACCAGCAAGTC | Antisense | 3368 |
| 244008_at | 485 | 285 | 144 | AATAGTGCTTATTTACATAGTCAAT | Antisense | 3369 |
| 244008_at | 163 | 41 | 193 | ATCTTCGATCTGCCCAATATTTAAT | Antisense | 3370 |
| 244008_at | 784 | 685 | 269 | GATATGCCTATGTTTCTTTAACTAT | Antisense | 3371 |
| 244008_at | 58 | 1011 | 283 | TCTTTAACTATACAGCCTCTTTACA | Antisense | 3372 |
| 244008_at | 121 | 721 | 338 | GTATTGCAACCTGCTATTTAGCCTT | Antisense | 3373 |
| 244008_at | 473 | 17 | 353 | ATTTAGCCTTTGGTGCCTTAGAATT | Antisense | 3374 |
| 244008_at | 872 | 1099 | 443 | TTTCTCTATTCACTTTTTATTGCTC | Antisense | 3375 |
| 244008_at | 199 | 7 | 461 | ATTGCTCTTGCTTTCTATTGCTACT | Antisense | 3376 |
| 244008_at | 591 | 509 | 480 | GCTACTAAAGCCTCTTTTATCCAGC | Antisense | 3377 |
| 244008_at | 316 | 739 | 508 | GTAATAGTTCCAACATTGTAGCGAA | Antisense | 3378 |
| 244272_s_at | 469 | 1081 | 49 | TATCATCATATATTTGTCCTCTAGC | Antisense | 3379 |
| 244272_s_at | 903 | 1157 | 62 | TTGTCCTCTAGCATGTATAACACCA | Antisense | 3380 |
| 244272_s_at | 888 | 109 | 87 | AGAGGGGACTCTAATGTAAACCATG | Antisense | 3381 |
| 244272_s_at | 903 | 669 | 128 | GATGTTTCAGAGTAGGTTCATTGAT | Antisense | 3382 |
| 244272_s_at | 282 | 201 | 190 | AAATAATCCTCAGTACAGCCAAGCT | Antisense | 3383 |
| 244272_s_at | 1139 | 733 | 202 | GTACAGCCAAGCTGAGGCTTTACAG | Antisense | 3384 |
| 244272_s_at | 193 | 1153 | 264 | TTGACTATTTGGGACTGCTGCTTTA | Antisense | 3385 |
| 244272_s_at | 126 | 1129 | 324 | TTCACCATAGAAACATCCTCCACAG | Antisense | 3386 |
| 244272_s_at | 94 | 199 | 360 | AAATTCTGTTGCCATTACATTCAAT | Antisense | 3387 |
| 244272_s_at | 1034 | 353 | 377 | CATTCAATTTCCAATATCCAGCAAA | Antisense | 3388 |
| 244272_s_at | 517 | 217 | 408 | AAACTTCCAATCTTAATATTAATCT | Antisense | 3389 |
| 244335_at | 552 | 51 | 22 | ATGCCAGAATTACCAAAAGTCACGA | Antisense | 3390 |
| 244335_at | 13 | 101 | 152 | AGTAGAGATTACCCAGCATTCAGCT | Antisense | 3391 |
| 244335_at | 437 | 325 | 172 | CAGCTCACCATTTGAAACCAGCCAA | Antisense | 3392 |
| 244335_at | 334 | 93 | 256 | AGTGTTACTCAATCTGGAAGTTGCA | Antisense | 3393 |
| 244335_at | 676 | 699 | 275 | GTTGCAATGAGCTTGACAATGTCAA | Antisense | 3394 |
| 244335_at | 257 | 645 | 312 | GAGATTCTCAATGAACCTCTAATGT | Antisense | 3395 |
| 244335_at | 751 | 575 | 324 | GAACCTCTAATGTTTTACCTTCAAT | Antisense | 3396 |
| 244335_at | 536 | 257 | 376 | AAGATTACCCAAATTCTACCATCAT | Antisense | 3397 |
| 244335_at | 167 | 1009 | 390 | TCTACCATCATTTTGCCACTGGGAT | Antisense | 3398 |
| 244335_at | 215 | 841 | 436 | GGATGACCATTATTATTCCTGGACT | Antisense | 3399 |
| 244335_at | 629 | 925 | 465 | TGTACACGATTCTCATAAACCCAAT | Antisense | 3400 |
| 244370_at | 1064 | 965 | 18 | TGCCCCCCTAGGTGCTAATAAAAGG | Antisense | 3401 |
| 244370_at | 807 | 651 | 140 | GAGTGTAGTGATCTTGTGCAAAAAA | Antisense | 3402 |
| 244370_at | 602 | 57 | 166 | ATGAACATTTGTGAGCTTCTTGCTG | Antisense | 3403 |
| 244370_at | 162 | 181 | 170 | ACATTTGTGAGCTTCTTGCTGTTTT | Antisense | 3404 |
| 244370_at | 707 | 943 | 177 | TGAGCTTCTTGCTGTTTTATAGATT | Antisense | 3405 |
| 244370_at | 681 | 1155 | 235 | TTGTTGTTCTTGTTGCGAAGGTTTC | Antisense | 3406 |
| 244370_at | 430 | 703 | 240 | GTTCTTGTTGCGAAGGTTTCAAATA | Antisense | 3407 |
| 244370_at | 133 | 1085 | 263 | TATTTGTGACTGAATGTACGGTGAA | Antisense | 3408 |
| 244370_at | 730 | 613 | 270 | GACTGAATGTACGGTGAAACTGTCA | Antisense | 3409 |
| 244370_at | 574 | 589 | 285 | GAAACTGTCATAATGTTACCTAGCT | Antisense | 3410 |
| 244370_at | 335 | 61 | 297 | ATGTTACCTAGCTGAGTTTTGTTAT | Antisense | 3411 |
| 244393_x_at | 880 | 421 | 132 | CCCAGCCGCTATAACTTTTAACAAT | Antisense | 3412 |
| 244393_x_at | 314 | 197 | 152 | ACAATTCCCATATGTCCTTTATTCC | Antisense | 3413 |
| 244393_x_at | 700 | 1077 | 162 | TATGTCCTTTATTCCACTAAGATGA | Antisense | 3414 |
| 244393_x_at | 464 | 59 | 183 | ATGAGTGCAGTATATATTTCCATCT | Antisense | 3415 |
| 244393_x_at | 489 | 23 | 196 | ATATTTCCATCTGTCCAAGGCTTCC | Antisense | 3416 |
| 244393_x_at | 796 | 795 | 270 | GGGCATTTGGGGAATCCAGGCTGTG | Antisense | 3417 |
| 244393_x_at | 1140 | 455 | 285 | CCAGGCTGTGATTCAGGGAAGTTCC | Antisense | 3418 |
| 244393_x_at | 597 | 257 | 303 | AAGTTCCAAGTGTCTGATGAAGTGT | Antisense | 3419 |
| 244393_x_at | 722 | 1155 | 328 | TTGTTTTACATCTTTGTGTCCCTTG | Antisense | 3420 |
| 244393_x_at | 247 | 453 | 348 | CCTTGCAGGTCTAGCACTGTGCTAT | Antisense | 3421 |
| 244393_x_at | 748 | 765 | 366 | GTGCTATGTAGGTAACATGTGCTCC | Antisense | 3422 |
| 35685_at | 1115 | 659 | 1036 | GAGGAGCCTGCTTTGCCCAGCCTGG | Antisense | 3423 |
| 35685_at | 886 | 79 | 1037 | AGGAGCCTGCTTTGCCCAGCCTGGA | Antisense | 3424 |
| 35685_at | 959 | 99 | 1079 | AGTACACCATCTACATCGCACCTGG | Antisense | 3425 |
| 35685_at | 252 | 191 | 1082 | ACACCATCTACATCGCACCTGGAGG | Antisense | 3426 |
| 35685_at | 199 | 1 | 1161 | ATTCTGGAAGGTGTCCCGGCCACTG | Antisense | 3427 |
| 35685_at | 486 | 463 | 1180 | CCACTGGAGCTGTGCTATGCTCCCA | Antisense | 3428 |
| 35685_at | 607 | 315 | 1181 | CACTGGAGCTGTGCTATGCTCCCAC | Antisense | 3429 |
| 35685_at | 391 | 421 | 1223 | CCCACCAGGGGACAGCCAGAGGAAG | Antisense | 3430 |
| 35685_at | 271 | 621 | 1250 | GACCATGGGGTATCCCTGTGTCCTG | Antisense | 3431 |
| 35685_at | 530 | 1079 | 1260 | TATCCCTGTGTCCTGGTCTATCACC | Antisense | 3432 |
| 35685_at | 607 | 489 | 1313 | GCCCAGCCAGCCAATAAGAGGACAC | Antisense | 3433 |
| 35685_at | 818 | 461 | 1319 | CCAGCCAATAAGAGGACACAAATGA | Antisense | 3434 |
| 35685_at | 1114 | 599 | 1333 | GACACAAATGAGGACACGTGGCTTT | Antisense | 3435 |
| 35685_at | 797 | 315 | 1347 | CACGTGGCTTTTATACAAAGTATCT | Antisense | 3436 |
| 35685_at | 425 | 395 | 1441 | CTGCAACGTCCCATCTATACGAGGT | Antisense | 3437 |
| 35685_at | 726 | 301 | 1444 | CAACGTCCCATCTATACGAGGTGTT | Antisense | 3438 |
| 39582_at | 184 | 891 | 2143 | GGCCACAAATGGAGGGATTGTCCTT | Antisense | 3439 |
| 39582_at | 965 | 759 | 2162 | GTCCTTTCAAGCACCACAGCTTCAG | Antisense | 3440 |
| 39582_at | 162 | 1113 | 2242 | TTATATTGGAGACTCAACTGCCCTT | Antisense | 3441 |
| 39582_at | 704 | 1115 | 2311 | TTAGAAAGTCTTAGCATCAGATCAT | Antisense | 3442 |
| 39582_at | 573 | 593 | 2314 | GAAAGTCTTAGCATCAGATCATAAA | Antisense | 3443 |
| 39582_at | 621 | 419 | 2361 | CCCATCTGAAACTTCCCAGGGGAGT | Antisense | 3444 |
| 39582_at | 880 | 453 | 2362 | CCATCTGAAACTTCCCAGGGGAGTT | Antisense | 3445 |
| 39582_at | 1069 | 1139 | 2373 | TTCCCAGGGGAGTTGGGATTCTTAG | Antisense | 3446 |
| 39582_at | 982 | 991 | 2374 | TCCCAGGGGAGTTGGGATTCTTAGT | Antisense | 3447 |
| 39582_at | 556 | 265 | 2410 | AAGGGGCTCATTTTCTACTGCATTT | Antisense | 3448 |
| 39582_at | 909 | 793 | 2413 | GGGCTCATTTTCTACTGCATTTCCC | Antisense | 3449 |
| 39582_at | 103 | 991 | 2434 | TCCCATTTTTGGTATCTTGTTCAGC | Antisense | 3450 |
| 39582_at | 289 | 425 | 2435 | CCCATTTTTGGTATCTTGTTCAGCA | Antisense | 3451 |
| 39582_at | 550 | 353 | 2437 | CATTTTTGGTATCTTGTTCAGCATG | Antisense | 3452 |

TABLE 16
100 PS probe sequences

| | | | | | | |
|---|---|---|---|---|---|---|
| | | | Probe | | | |
| Probe Set | | | Interrogation | | Target | |
| Name | Probe.X | Probe.Y | Position | Probe.Sequence | Strandedness | SEQ ID NO: |

| | | | | | | |
|---|---|---|---|---|---|---|
| 1555852_at | 9 | 453 | 729 | CCATTCTGAGTACTTCTCCGCAAAC | Antisense | 3453 |
| 1555852_at | 567 | 615 | 770 | GACTGTTTTACATGAAGGGTGCAAA | Antisense | 3454 |
| 1555852_at | 99 | 1 | 811 | GAACCCTAGGGTGAAACACGTGACA | Antisense | 3455 |
| 1555852_at | 208 | 1089 | 849 | TATTGAATAGTCCTCTTCTCTACCC | Antisense | 3456 |
| 1555852_at | 446 | 255 | 926 | AAGAGATGAGCATTCAAGTCAGGCA | Antisense | 3457 |
| 1555852_at | 412 | 955 | 955 | TGAATTTGGGTCAAGGCTGCGCCAC | Antisense | 3458 |
| 1555852_at | 132 | 75 | 968 | AGGCTGCGCCACTCAAAAGCTATAT | Antisense | 3459 |
| 1555852_at | 158 | 25 | 989 | ATATGACCTCTATATGAGCAGCTTA | Antisense | 3460 |
| 1555852_at | 1121 | 1087 | 1012 | TATTCAACCTCTTTTAACCTCCATT | Antisense | 3461 |
| 1555852_at | 341 | 243 | 1027 | AACCTCCATTTTGTCATCTGTAGAA | Antisense | 3462 |
| 1555852_at | 310 | 1049 | 1058 | TAAATGCCTAGCTCAGAAGGATTCC | Antisense | 3463 |
| 1556049_at | 671 | 213 | 2585 | AAACTCCAGGGCTCATTAGTCCTTT | Antisense | 3464 |
| 1556049_at | 873 | 203 | 2626 | AAATGATGGGTATCCGTGTGCCTGG | Antisense | 3465 |
| 1556049_at | 59 | 907 | 2648 | TGGTCCAAGTTTCCTGGTGGTTGAC | Antisense | 3466 |
| 1556049_at | 136 | 1041 | 2688 | TAAGACATGTTCTGCAGAGTTCTCC | Antisense | 3467 |
| 1556049_at | 458 | 741 | 2727 | GTAATATTCCGTTTGTTTTGCTCCT | Antisense | 3468 |
| 1556049_at | 536 | 633 | 2808 | GAGCAACGATCATTTTCCTAGTCAA | Antisense | 3469 |
| 1556049_at | 160 | 521 | 2869 | GCATTAAACTATAAATCCTCCCTCC | Antisense | 3470 |
| 1556049_at | 791 | 57 | 2930 | ATGACAATGCCTACTGGGCTCAGCT | Antisense | 3471 |
| 1556049_at | 314 | 941 | 2966 | TGAGTCTGACTGAGCCTTCTTAGCA | Antisense | 3472 |
| 1556049_at | 769 | 455 | 3060 | CCAGGTATTTTCAGCAGAGGTTCTT | Antisense | 3473 |
| 1556049_at | 276 | 1055 | 3084 | TACTTTTCTGTAATTGAGGCGCAAC | Antisense | 3474 |
| 1561042_at | 939 | 203 | 444 | AAATGCAGATAAAGGCTACCTCTGA | Antisense | 3475 |
| 1561042_at | 950 | 1047 | 453 | TAAAGGCTACCTCTGAATTCTCAAT | Antisense | 3476 |
| 1561042_at | 156 | 207 | 454 | AAAGGCTACCTCTGAATTCTCAATA | Antisense | 3477 |
| 1561042_at | 287 | 75 | 456 | AGGCTACCTCTGAATTCTCAATAGA | Antisense | 3478 |
| 1561042_at | 640 | 549 | 467 | GAATTCTCAATAGATTCATCATGTT | Antisense | 3479 |
| 1561042_at | 390 | 285 | 475 | AATAGATTCATCATGTTTGCTCTTA | Antisense | 3480 |
| 1561042_at | 971 | 691 | 479 | GATTCATCATGTTTGCTCTTAAGTG | Antisense | 3481 |
| 1561042_at | 921 | 1015 | 482 | TCATCATGTTTGCTCTTAAGTGTAG | Antisense | 3482 |
| 1561042_at | 76 | 719 | 489 | GTTTGCTCTTAAGTGTAGCTGTCCA | Antisense | 3483 |
| 1561042_at | 390 | 963 | 492 | TGCTCTTAAGTGTAGCTGTCCACAC | Antisense | 3484 |
| 1561042_at | 902 | 379 | 494 | CTCTTAAGTGTAGCTGTCCACACTG | Antisense | 3485 |
| 1563497_at | 251 | 1063 | 4804 | TACCATAGCAAGTTCTTATCCTTTG | Antisense | 3486 |
| 1563497_at | 173 | 157 | 4861 | ACCTGTCACAGTAATATGCCAATTT | Antisense | 3487 |
| 1563497_at | 943 | 467 | 4878 | GCCAATTTTTGGTGTCGCTTTAGTA | Antisense | 3488 |
| 1563497_at | 834 | 749 | 4965 | GTCTTTCCTCAGTTGTCTCCTAAAA | Antisense | 3489 |
| 1563497_at | 555 | 677 | 5022 | GATCGTCTTGTGCAGCCCTTTAAAA | Antisense | 3490 |
| 1563497_at | 941 | 53 | 5048 | ATGCGTTTTGCATTCATGTCTTTGC | Antisense | 3491 |
| 1563497_at | 920 | 365 | 5088 | CTTTCATTTTGGGAGTGACTGTCAT | Antisense | 3492 |
| 1563497_at | 1079 | 189 | 5141 | ACACCAGGGCCAGATTGCGTGGGTT | Antisense | 3493 |
| 1563497_at | 610 | 965 | 5156 | TGCGTGGGTTCTAGCTAGGGACCTA | Antisense | 3494 |
| 1563497_at | 1053 | 141 | 5182 | AGCTGGTTACTTGATTTCTCTCTGT | Antisense | 3495 |
| 1563497_at | 93 | 1137 | 5197 | TTCTCTCTGTTCCATTTACTATCTG | Antisense | 3496 |
| 200615_s_at | 715 | 795 | 2839 | GGGCAGGACATGCTGTACCAATCCC | Antisense | 3497 |
| 200615_s_at | 356 | 1063 | 2854 | TACCAATCCCTGAAGCTCACTAATG | Antisense | 3498 |
| 200615_s_at | 1122 | 889 | 2891 | TGGCCGAACTACGTATCCAGCCAGG | Antisense | 3499 |
| 200615_s_at | 858 | 305 | 2908 | CAGCCAGGAAACCCCAATTACACGC | Antisense | 3500 |
| 200615_s_at | 511 | 629 | 2948 | GAGCTCCTGAAGTCTCTCAATACAT | Antisense | 3501 |
| 200615_s_at | 1143 | 31 | 2967 | ATACATCTATCAGGTCTACGACAGC | Antisense | 3502 |
| 200615_s_at | 364 | 297 | 3007 | CAAGACTGGTCCAGTACCCTTCAAC | Antisense | 3503 |
| 200615_s_at | 13 | 161 | 3022 | ACCCTTCAACCATGCTGTGATCGGT | Antisense | 3504 |
| 200615_s_at | 1108 | 737 | 3125 | GTAACTAGTCTAACCTGTGCTAACA | Antisense | 3505 |
| 200615_s_at | 468 | 685 | 3168 | GATAGTTTTAGCTTCCTGTGAACAT | Antisense | 3506 |
| 200615_s_at | 108 | 469 | 3276 | GCCAATGGCTGGCTTTTTCTACACC | Antisense | 3507 |
| 200904_at | 733 | 919 | 1987 | TGTGCCTTCATTCATGGGTTAATGG | Antisense | 3508 |
| 200904_at | 842 | 267 | 2015 | AATGGGTTATCACAGGAATGGGACT | Antisense | 3509 |
| 200904_at | 754 | 115 | 2063 | AGAGAACTGAGCTAGCATGCCCAGC | Antisense | 3510 |
| 200904_at | 296 | 421 | 2087 | CCCACAGAGAGCCTCCACTAGAGTG | Antisense | 3511 |
| 200904_at | 877 | 625 | 2095 | GAGCCTCCACTAGAGTGATGCTAAG | Antisense | 3512 |
| 200904_at | 590 | 749 | 2168 | GTCTAGTGTCTAGTGGATCCAGGCC | Antisense | 3513 |
| 200904_at | 756 | 839 | 2182 | GGATCCAGGCCACAGGAGAGAGTGC | Antisense | 3514 |
| 200904_at | 1010 | 237 | 2253 | AACTGTGGAGTCAGTGGCAGCATGC | Antisense | 3515 |
| 200904_at | 421 | 211 | 2292 | AAAGCTTTAGGCACCAGCCTGCAAC | Antisense | 3516 |
| 200904_at | 729 | 415 | 2322 | CGAGCAGCCACGTAGGCTGCACCCA | Antisense | 3517 |
| 200904_at | 806 | 451 | 2506 | CCTTCCCAGGCTGATCTGAGGTAAA | Antisense | 3518 |
| 200905_x_at | 720 | 213 | 1095 | AAAGCCTGAGACAGCTGCCTTGTGT | Antisense | 3519 |
| 200905_x_at | 1116 | 415 | 1121 | CGACTGAGATGCACAGCTGCCTTGT | Antisense | 3520 |
| 200905_x_at | 1111 | 503 | 1136 | GCTGCCTTGTGTGCGACTGAGATGC | Antisense | 3521 |
| 200905_x_at | 757 | 941 | 1153 | TGAGATGCAGGATTTCCTCACGCCT | Antisense | 3522 |
| 200905_x_at | 919 | 65 | 1183 | ATGTGTCTTAGGGGACTCTGGCTTC | Antisense | 3523 |
| 200905_x_at | 504 | 879 | 1202 | GGCTTCTCTTTTTGCAAGGGCCTCT | Antisense | 3524 |
| 200905_x_at | 369 | 999 | 1230 | TCTGTCTGTGTCCCTGTTAGCACAA | Antisense | 3525 |
| 200905_x_at | 608 | 115 | 1266 | AGAGAAACAGTCCACCTCTGTGTCT | Antisense | 3526 |
| 200905_x_at | 1142 | 913 | 1360 | TGGGCAGAGTGCGGCAGCTCATGCC | Antisense | 3527 |
| 200905_x_at | 917 | 771 | 1587 | GTGAGCCAGGATTGTGCGACTGCAC | Antisense | 3528 |
| 200905_x_at | 728 | 809 | 1620 | GGGTGACAGGGTGAAACGCCATCTC | Antisense | 3529 |
| 201009_s_at | 311 | 31 | 1461 | ATACTGAGGTGGATCCCTGCATCCT | Antisense | 3530 |
| 201009_s_at | 1005 | 427 | 1475 | CCCTGCATCCTCAACAACAATGTGC | Antisense | 3531 |
| 201009_s_at | 153 | 571 | 1520 | GAAGCAGCTTTACCTACTTGTTTCT | Antisense | 3532 |
| 201009_s_at | 57 | 365 | 1615 | CTTAGCCTCTGACTTCCTAATGTAG | Antisense | 3533 |
| 201009_s_at | 954 | 843 | 1639 | GGAGGTGGTCAGCAGGCAATCTCCT | Antisense | 3534 |
| 201009_s_at | 280 | 869 | 1653 | GGCAATCTCCTGGGCCTTAAAGGAT | Antisense | 3535 |
| 201009_s_at | 57 | 207 | 1671 | AAAGGATGCGGACTCATCCTCAGCC | Antisense | 3536 |
| 201009_s_at | 352 | 461 | 1694 | CCAGCGCCCATGTTGTGATACAGGG | Antisense | 3537 |
| 201009_s_at | 988 | 585 | 1749 | GAAAAACTCAGGCCCATCCATTTTC | Antisense | 3538 |
| 201009_s_at | 497 | 289 | 1789 | AATTGAGGCCTTTTCGATAGTTTCG | Antisense | 3539 |
| 201009_s_at | 245 | 205 | 1824 | AAATGGCCTCCTGGCGTAAGCTTTT | Antisense | 3540 |
| 201010_s_at | 1118 | 775 | 2116 | GTGTTCTCCTACTGCAAATATTTTC | Antisense | 3541 |
| 201010_s_at | 612 | 829 | 2153 | GGTTTTCTCTTCATGTAAGTCCTTG | Antisense | 3542 |
| 201010_s_at | 776 | 931 | 2196 | TGTTCTTAGCACTTTAATTCCTGTC | Antisense | 3543 |
| 201010_s_at | 968 | 499 | 2259 | GCTGAAACTGGTCTACTGTGTCTCT | Antisense | 3544 |
| 201010_s_at | 909 | 593 | 2373 | GAAAGGGTTGCTGCTGTCAGCCTTG | Antisense | 3545 |
| 201010_s_at | 538 | 301 | 2444 | CAACCCTGTGATCAGAACCTCCAAA | Antisense | 3546 |
| 201010_s_at | 777 | 107 | 2540 | AGATAGGGATATTGGCCCCTCACTG | Antisense | 3547 |
| 201010_s_at | 1097 | 313 | 2587 | CACTCTCAGCCATAGCACTTTGTTC | Antisense | 3548 |
| 201010_s_at | 814 | 175 | 2603 | ACTTTGTTCACTGTCCTGTGTCAGA | Antisense | 3549 |
| 201010_s_at | 668 | 917 | 2619 | TGTGTCAGAGCACTGAGCTCCACCC | Antisense | 3550 |
| 201010_s_at | 799 | 139 | 2634 | AGCTCCACCCTTTTCTGAGAGTTAT | Antisense | 3551 |
| 202544_at | 797 | 719 | 3563 | GTATTCTTACTATGGTTGCCTTTCC | Antisense | 3552 |
| 202544_at | 520 | 1147 | 3578 | TTGCCTTTCCCTATTTGTTCAATAG | Antisense | 3553 |
| 202544_at | 495 | 63 | 3703 | ATGTAGTAATCTTCTAGACCGCTTA | Antisense | 3554 |
| 202544_at | 672 | 119 | 3718 | AGACCGCTTAAAACCAATGCTCCCA | Antisense | 3555 |
| 202544_at | 665 | 291 | 3732 | CAATGCTCCCAAGCTGAATATTCTT | Antisense | 3556 |
| 202544_at | 22 | 645 | 3891 | GAGATCATTAACATGCCAGGGCAGT | Antisense | 3557 |
| 202544_at | 840 | 457 | 3906 | CCAGGGCAGTTCCCACTGATTTAGA | Antisense | 3558 |
| 202544_at | 979 | 229 | 3992 | AACAGTTCTCTGTCATCGTTTAACA | Antisense | 3559 |
| 202544_at | 47 | 195 | 4023 | ACAACTGACACAACTCCTTAAGCTG | Antisense | 3560 |
| 202544_at | 173 | 1041 | 4041 | TAAGCTGTGGTTTCAGTCTCTGCTA | Antisense | 3561 |
| 202544_at | 320 | 751 | 4056 | GTCTCTGCTAGTTCATATTGCATGT | Antisense | 3562 |
| 202546_at | 849 | 675 | 67 | GATCTGGTGCGGAACCTGCAAAGTG | Antisense | 3563 |
| 202546_at | 1110 | 421 | 119 | CCCAGAATGTGGAGCGGATCCTGGC | Antisense | 3564 |
| 202546_at | 90 | 585 | 151 | GAAAACTTGGAACATCTCCGCAACA | Antisense | 3565 |
| 202546_at | 1046 | 579 | 160 | GAACATCTCCGCAACAAGACAGAGG | Antisense | 3566 |
| 202546_at | 503 | 659 | 181 | GAGGATCTGGAAGCCACATCTGAGC | Antisense | 3567 |
| 202546_at | 497 | 245 | 191 | AAGCCACATCTGAGCACTTCAAGAC | Antisense | 3568 |
| 202546_at | 274 | 631 | 202 | GAGCACTTCAAGACGACATCGCAGA | Antisense | 3569 |
| 202546_at | 1001 | 617 | 213 | GACGACATCGCAGAAGGTGGCTCGA | Antisense | 3570 |
| 202546_at | 549 | 265 | 226 | AAGGTGGCTCGAAAATTCTGGTGGA | Antisense | 3571 |
| 202546_at | 152 | 671 | 261 | GATGATTGTCCTTATCTGCGTGATT | Antisense | 3572 |
| 202546_at | 672 | 463 | 320 | CCACTGGTGCCTTCTCTTAAAGTAA | Antisense | 3573 |
| 202828_s_at | 409 | 811 | 2994 | GGGGGCCACAGGGTGGGCTGTGGAG | Antisense | 3574 |
| 202828_s_at | 721 | 915 | 3012 | TGTGGAGCTCTCAGGAAGGGCCCTG | Antisense | 3575 |
| 202828_s_at | 129 | 399 | 3034 | CTGAGGAAGGCACACTTGCTCCTGT | Antisense | 3576 |
| 202828_s_at | 282 | 939 | 3146 | TGAGGGGCTTCACGAGAGGCCACAG | Antisense | 3577 |
| 202828_s_at | 285 | 481 | 3195 | GCCTGCTCACCATCTCAGTGAGGGG | Antisense | 3578 |
| 202828_s_at | 238 | 421 | 3320 | CCCACCCATTGAAGTCTCCTTGGGC | Antisense | 3579 |
| 202828_s_at | 936 | 751 | 3333 | GTCTCCTTGGGCCACCAAAGGTGGT | Antisense | 3580 |
| 202828_s_at | 990 | 893 | 3357 | TGGCCATGGTACCGGGGACTTGGGA | Antisense | 3581 |
| 202828_s_at | 667 | 109 | 3406 | AGAGGAGAGGGATGTCGGGGGGGTG | Antisense | 3582 |
| 202828_s_at | 938 | 593 | 3446 | GAAATGGGGTGAACGGTGCTGGCAG | Antisense | 3583 |
| 202828_s_at | 951 | 763 | 3461 | GTGCTGGCAGTTCGGCTAGATTTCT | Antisense | 3584 |
| 204078_at | 1122 | 233 | 2016 | AACTTAAGATGGACACAGCTGACTG | Antisense | 3585 |
| 204078_at | 339 | 111 | 2022 | AGATGGACACAGCTGACTGGACCCC | Antisense | 3586 |
| 204078_at | 40 | 313 | 2091 | CACTTCTATGTCTCTGGACCACAGG | Antisense | 3587 |
| 204078_at | 125 | 871 | 2137 | GGCAAGTGGGCTGATGGGGTCCGCC | Antisense | 3588 |
| 204078_at | 895 | 751 | 2192 | GTCTCCTGGACAAGGAGTTTCCAGC | Antisense | 3589 |
| 204078_at | 470 | 439 | 2196 | CCTGGACAAGGAGTTTCCAGCTGCT | Antisense | 3590 |
| 204078_at | 460 | 885 | 2221 | GGCTGGAGTCTCAGGCCAAATTGCA | Antisense | 3591 |
| 204078_at | 55 | 73 | 2233 | AGGCCAAATTGCAGAGGGTCCTCCA | Antisense | 3592 |
| 204078_at | 1110 | 337 | 2256 | CAGGGTCCTGAAGAGCACTGGACTA | Antisense | 3593 |
| 204078_at | 1062 | 441 | 2262 | CCTGAAGAGCACTGGACTAAGAGTC | Antisense | 3594 |
| 204078_at | 250 | 173 | 2277 | ACTAAGAGTCTAGTGGTTCCAGGGC | Antisense | 3595 |
| 204116_at | 475 | 1137 | 867 | TTCTGGCTGGAACGGACGATGCCCC | Antisense | 3596 |
| 204116_at | 606 | 821 | 917 | GGATCTTGTTACTGAATACCACGGG | Antisense | 3597 |
| 204116_at | 110 | 817 | 960 | GGTGTGTCTAAGGGACTGGCTGAGA | Antisense | 3598 |
| 204116_at | 902 | 173 | 997 | ACTACAGTGAACGACTCTGCCTCGT | Antisense | 3599 |
| 204116_at | 121 | 613 | 1009 | GACTCTGCCTCGTCAGTGAGATTCC | Antisense | 3600 |
| 204116_at | 998 | 707 | 1111 | GTTACACCCTAAAGCCTGAAACCTG | Antisense | 3601 |
| 204116_at | 845 | 241 | 1136 | AACCCCAATCCTCTGACAGAAGAAC | Antisense | 3602 |
| 204116_at | 309 | 819 | 1166 | GGTCCTGTAGCCCTAAGTGGTACTA | Antisense | 3603 |
| 204116_at | 1123 | 781 | 1182 | GTGGTACTAACTTTCCTTCATTCAA | Antisense | 3604 |
| 204116_at | 877 | 883 | 1242 | GGCTGATTTGGAATTTTGTGCCCCC | Antisense | 3605 |
| 204116_at | 453 | 285 | 1253 | AATTTTGTGCCCCCATGTAAGCACC | Antisense | 3606 |
| 204233_s_at | 172 | 543 | 1157 | GCAAACATCCGGAAGTATCCCACCA | Antisense | 3607 |
| 204233_s_at | 743 | 1079 | 1172 | TATCCCACCAAGAAACAACAGCTCC | Antisense | 3608 |
| 204233_s_at | 950 | 1085 | 1201 | TATTTCCAGTTACTTGCCTGCATTC | Antisense | 3609 |
| 204233_s_at | 431 | 1035 | 1294 | TAATAGGTTTGCCCTTGCATCTCAT | Antisense | 3610 |
| 204233_s_at | 378 | 801 | 1327 | GGGACTGTGGTCCATTGTACAAGCC | Antisense | 3611 |
| 204233_s_at | 60 | 549 | 1367 | GAATTTGGGTACATGGACTACGCCC | Antisense | 3612 |
| 204233_s_at | 307 | 857 | 1381 | GGACTACGCCCAAGCAAGGTTTGAT | Antisense | 3613 |
| 204233_s_at | 803 | 565 | 1426 | GAAGCTTGGGGTGTGACTGTGGGGA | Antisense | 3614 |
| 204233_s_at | 152 | 155 | 1462 | ACCTCATCACTGGACTGCATGGGGA | Antisense | 3615 |
| 204233_s_at | 742 | 789 | 1530 | GTGGCAGGAGGCTTTGGGTGGCTCA | Antisense | 3616 |
| 204233_s_at | 936 | 1091 | 1542 | TTTGGGTGGCTCACTACTGAACACA | Antisense | 3617 |
| 204266_s_at | 680 | 927 | 1821 | TGTAGATGGCGACTGTGTTATAGAC | Antisense | 3618 |
| 204266_s_at | 802 | 927 | 1836 | TGTTATAGACGGTGCTCACTCCCAA | Antisense | 3619 |
| 204266_s_at | 227 | 669 | 1880 | GATGTACTGCAAAGTCGCCAGTCAC | Antisense | 3620 |
| 204266_s_at | 929 | 471 | 1896 | GCCAGTCACTGCGTGCAAGGTGGCC | Antisense | 3621 |
| 204266_s_at | 276 | 133 | 1980 | AGCAGCTGGATTGCTCTGGAGCAGC | Antisense | 3622 |
| 204266_s_at | 395 | 919 | 2116 | TGTGCAGACAGCCATGCGTGGCCAC | Antisense | 3623 |
| 204266_s_at | 56 | 971 | 2232 | TGCCTGGAGTGTCGGATCCTGTGAG | Antisense | 3624 |
| 204266_s_at | 63 | 653 | 2238 | GAGTGTCGGATCCTGTGAGTAGGCT | Antisense | 3625 |
| 204266_s_at | 121 | 429 | 2323 | CCCTGACATCATCATTCTTGTGGGA | Antisense | 3626 |
| 204266_s_at | 918 | 801 | 2344 | GGGAGACAGCAGCCTGTATGTGGTG | Antisense | 3627 |
| 204266_s_at | 372 | 783 | 2366 | GTGTGGGGCGTGGATCGAGTGTAGC | Antisense | 3628 |
| 204410_at | 165 | 449 | 849 | CCTTTCCTACTGTGATAACGTCAAG | Antisense | 3629 |
| 204410_at | 114 | 553 | 888 | GAATACATTTCTGCCTGATAATCAT | Antisense | 3630 |
| 204410_at | 551 | 971 | 899 | TGCCTGATAATCATGCTGAGTTCTA | Antisense | 3631 |
| 204410_at | 537 | 941 | 915 | TGAGTTCTAATAAGCCCTACTTCCA | Antisense | 3632 |
| 204410_at | 443 | 373 | 931 | CTACTTCCACCTAATCTGTTTACAG | Antisense | 3633 |
| 204410_at | 865 | 715 | 967 | GTTTCAGTTACTTAGATGGTCTCAT | Antisense | 3634 |
| 204410_at | 346 | 667 | 981 | GATGGTCTCATAAGGTTTCTGATAC | Antisense | 3635 |
| 204410_at | 835 | 1017 | 1115 | TCATCCTTTGTGCCTCGGTTATTAA | Antisense | 3636 |
| 204410_at | 807 | 561 | 1223 | GAAACCGAACAATAAGTGGCAACCA | Antisense | 3637 |
| 204410_at | 95 | 859 | 1262 | GGAAATACTGGGGTGCCATTTTGTT | Antisense | 3638 |
| 204410_at | 463 | 1155 | 1302 | TTGTAATCCACTGTTTTGGCTTTCA | Antisense | 3639 |
| 204806_x_at | 716 | 329 | 620 | CAGATCCTCCAAAGGCACACGTTGC | Antisense | 3640 |
| 204806_x_at | 920 | 1007 | 698 | TCTACCCTGCGGAGATCACGCTGAC | Antisense | 3641 |
| 204806_x_at | 353 | 863 | 741 | GGAACAGACCCAGGACACAGAGCTT | Antisense | 3642 |
| 204806_x_at | 319 | 329 | 758 | CAGAGCTTGTGGAGACCAGGCCTGC | Antisense | 3643 |
| 204806_x_at | 622 | 73 | 775 | AGGCCTGCAGGGGATGGAACCTTCC | Antisense | 3644 |
| 204806_x_at | 575 | 323 | 877 | CAGCCCCTCATCCTGAGATGGGAGC | Antisense | 3645 |
| 204806_x_at | 693 | 929 | 948 | TGTTGTCCTTGGAGCTGTGGTCACT | Antisense | 3646 |
| 204806_x_at | 909 | 403 | 977 | CTGTGGTCGCTGCTGTGATGTGGAG | Antisense | 3647 |
| 204806_x_at | 992 | 381 | 1035 | CTCTCAGGCTGCAGTCACTGACAGT | Antisense | 3648 |
| 204806_x_at | 406 | 315 | 1050 | CACTGACAGTGCCCAGGGCTCTGGG | Antisense | 3649 |
| 204806_x_at | 68 | 773 | 1098 | GTGAGACAGCTTCCTTGTGTGGGAC | Antisense | 3650 |
| 204897_at | 294 | 133 | 3030 | AGCAGCTTATTGTTTCTCTGAAAGT | Antisense | 3651 |
| 204897_at | 401 | 71 | 3112 | ATGGCAAGTTGCATCAGAAAGCTTT | Antisense | 3652 |
| 204897_at | 1120 | 685 | 3154 | GATTCCCAAACGTGGTTACATTAGC | Antisense | 3653 |
| 204897_at | 736 | 239 | 3162 | AACGTGGTTACATTAGCCATTCATG | Antisense | 3654 |
| 204897_at | 267 | 475 | 3177 | GCCATTCATGTATGTCAGAAGTGCA | Antisense | 3655 |
| 204897_at | 1002 | 909 | 3206 | TGGGGCACTTAATGGTCACCTTGTA | Antisense | 3656 |
| 204897_at | 560 | 735 | 3228 | GTAACAGTTTTGTGTAACTCCCAGT | Antisense | 3657 |
| 204897_at | 102 | 739 | 3241 | GTAACTCCCAGTGATGCTGTACACA | Antisense | 3658 |
| 204897_at | 629 | 499 | 3256 | GCTGTACACATATTTGAAGGGTCTT | Antisense | 3659 |
| 204897_at | 692 | 777 | 3315 | GTGTTTTTGTGAATTGCTTGGTTGT | Antisense | 3660 |
| 204897_at | 1018 | 197 | 3344 | AAATTCTGAGCCTGATATTGATATG | Antisense | 3661 |
| 205027_s_at | 290 | 15 | 2203 | ATTATATAACTCTTTGAGCCTTTAT | Antisense | 3662 |
| 205027_s_at | 997 | 625 | 2218 | GAGCCTTTATTGGTAAATTCTGGTA | Antisense | 3663 |
| 205027_s_at | 81 | 287 | 2260 | AATTTGGGTGACTAGAACAACTTGA | Antisense | 3664 |
| 205027_s_at | 652 | 729 | 2290 | GTAGCAATAAGCTGGACTAGTGTCC | Antisense | 3665 |
| 205027_s_at | 158 | 779 | 2309 | GTGTCCTAAAAATGGCTAACTGATG | Antisense | 3666 |
| 205027_s_at | 956 | 769 | 2364 | GTGACAGTTTCTTTTGTGTTCCTAT | Antisense | 3667 |
| 205027_s_at | 91 | 703 | 2381 | GTTCCTATGGAAACATTTTATACTG | Antisense | 3668 |
| 205027_s_at | 38 | 917 | 2599 | TGTGTATGTTTTATATCAAATGCCT | Antisense | 3669 |
| 205027_s_at | 22 | 1031 | 2614 | TCAAATGCCTTCATGAATCTTTCAT | Antisense | 3670 |
| 205027_s_at | 667 | 215 | 2712 | AAACCCAATACTTTTGTCCAATGTG | Antisense | 3671 |
| 205027_s_at | 1156 | 175 | 2721 | ACTTTTGTCCAATGTGGTTGGTCAA | Antisense | 3672 |
| 205204_at | 363 | 109 | 167 | AGATCCGAGTGCACTCGCGAGGCAA | Antisense | 3673 |
| 205204_at | 229 | 463 | 200 | CCACCGGTCACTTCATGGGCAAGAA | Antisense | 3674 |
| 205204_at | 297 | 801 | 281 | GGGACCAGCGACTGCAGCTGAGTCA | Antisense | 3675 |
| 205204_at | 580 | 649 | 300 | GAGTCATGATCTGCTCGGAATCCTC | Antisense | 3676 |
| 205204_at | 324 | 963 | 311 | TGCTCGGAATCCTCCTGCTAAAGAA | Antisense | 3677 |
| 205204_at | 608 | 535 | 364 | GCACCCCAAATCCAGTACAGGAGGC | Antisense | 3678 |
| 205204_at | 882 | 1073 | 424 | TAGGGGCAGACACAACAGCGTGGCT | Antisense | 3679 |
| 205204_at | 829 | 141 | 440 | AGCGTGGCTTAGATTGTGCCCACCC | Antisense | 3680 |
| 205204_at | 311 | 763 | 455 | GTGCCCACCCAGGGAAGGTGCTGAA | Antisense | 3681 |
| 205204_at | 1159 | 951 | 476 | TGAATGGGACCCTGTTGATGGCCCC | Antisense | 3682 |
| 205204_at | 541 | 279 | 525 | AATCTCTGTTACTCCATTACTGTGA | Antisense | 3683 |
| 205273_s_at | 536 | 883 | 1991 | GGCTGTCGACTGGGCTAAGTCTGGA | Antisense | 3684 |
| 205273_s_at | 369 | 993 | 2035 | TCGACGAAGCCAAACTTTCTGTCTT | Antisense | 3685 |
| 205273_s_at | 329 | 1139 | 2051 | TTCTGTCTTCTCAACCGTAGATGCT | Antisense | 3686 |
| 205273_s_at | 525 | 985 | 2075 | TCCTGTCGCTCCTTCAGACAAAGGA | Antisense | 3687 |
| 205273_s_at | 190 | 265 | 2095 | AAGGAATGGACCACTTCTTGTACGG | Antisense | 3688 |
| 205273_s_at | 422 | 1013 | 2110 | TCTTGTACGGCCTCTCGGATGAGAT | Antisense | 3689 |
| 205273_s_at | 788 | 363 | 2159 | CTTTGCTGTCAGCCACGACAAGCTC | Antisense | 3690 |
| 205273_s_at | 632 | 539 | 2314 | GCACAGGAGCCCGAGACAATACACC | Antisense | 3691 |
| 205273_s_at | 984 | 923 | 2392 | TGTCATTGAGTGTCGCCAAAGCCCT | Antisense | 3692 |
| 205273_s_at | 1050 | 467 | 2406 | GCCAAAGCCCTTGACTGGCGAGTCA | Antisense | 3693 |
| 205273_s_at | 590 | 1109 | 2516 | TTTTCACTTTGTACTGGCAGGCTCG | Antisense | 3694 |
| 205419_at | 665 | 323 | 1055 | CAGCCCCTGAAGAAAATTCACGTGA | Antisense | 3695 |
| 205419_at | 139 | 671 | 1095 | GATGATGATACATTCCAAGTCTTCA | Antisense | 3696 |
| 205419_at | 477 | 599 | 1171 | GACAAACTTTGCAGGACTTCCCTTA | Antisense | 3697 |
| 205419_at | 288 | 525 | 1181 | GCAGGACTTCCCTTATAAAGCAAAA | Antisense | 3698 |
| 205419_at | 1029 | 705 | 1211 | GTTCAGCTTCCAATTAGTATTCTTT | Antisense | 3699 |
| 205419_at | 465 | 1085 | 1236 | TATATTTCTTTCATTGGGCGCTTTC | Antisense | 3700 |
| 205419_at | 228 | 365 | 1256 | CTTTCCCATCTCCAACTCGGAAGTA | Antisense | 3701 |
| 205419_at | 104 | 565 | 1275 | GAAGTAAGCCCAAGAGAACAACATA | Antisense | 3702 |
| 205419_at | 952 | 551 | 1361 | GAATACACCAAAAGGAGGCGCTCTT | Antisense | 3703 |
| 205419_at | 451 | 1 | 1526 | AAATCCAAATTTCTCTCAATGTTAG | Antisense | 3704 |
| 205419_at | 469 | 649 | 1550 | GATTTAATTCCCTCAATAACACCAA | Antisense | 3705 |
| 205696_s_at | 858 | 1143 | 2023 | TTCCTGTTCTCTTGTATAGCTGAAA | Antisense | 3706 |
| 205696_s_at | 927 | 703 | 2028 | GTTCTCTTGTATAGCTGAAATTCCA | Antisense | 3707 |
| 205696_s_at | 669 | 597 | 2044 | GAAATTCCAGTTTAGGAGCTCAGTT | Antisense | 3708 |
| 205696_s_at | 1014 | 635 | 2069 | GAGAAACAGTTCCATTCAACTGGAA | Antisense | 3709 |
| 205696_s_at | 17 | 951 | 2146 | TGAAAAACCTGATGCAGTGCTCCAT | Antisense | 3710 |
| 205696_s_at | 491 | 799 | 2209 | GGGACAGTTTGTAACTTGGGCTGTA | Antisense | 3711 |
| 205696_s_at | 666 | 1041 | 2344 | TAACTCCCATATCTCCTTTAATGAC | Antisense | 3712 |
| 205696_s_at | 299 | 425 | 2349 | CCCATATCTCCTTTAATGACATTGA | Antisense | 3713 |
| 205696_s_at | 247 | 5 | 2401 | ATTGATGCCAAGCTTTTTTGCCACA | Antisense | 3714 |
| 205696_s_at | 68 | 937 | 2403 | TGATGCCAAGCTTTTTTGCCACAAA | Antisense | 3715 |
| 205696_s_at | 1048 | 793 | 2447 | GGGCTTTGTGGAAACAGCTGGTACT | Antisense | 3716 |
| 205890_s_at | 632 | 625 | 195 | GATCTTAAAGCCACGGAGAAGCCTC | Antisense | 3717 |
| 205890_s_at | 247 | 969 | 293 | TGCCCTTGTTTCTTGTGGAGTCAGG | Antisense | 3718 |
| 205890_s_at | 584 | 59 | 320 | ATGAGGCAAAGAGGCACCTCCTCCA | Antisense | 3719 |
| 205890_s_at | 578 | 561 | 350 | GAAGGTCCAGCTCAGTGGCACAAGT | Antisense | 3720 |
| 205890_s_at | 767 | 615 | 396 | GACGGGTATAATCCCTGAGACCCAG | Antisense | 3721 |
| 205890_s_at | 615 | 619 | 414 | GACCCAGATTGTGACTTGCAATGGA | Antisense | 3722 |
| 205890_s_at | 783 | 113 | 481 | AGAAAGGGCAACTTACTCTTCCTGG | Antisense | 3723 |
| 205890_s_at | 67 | 173 | 495 | ACTCTTCCTGGCATCTTATTGTATT | Antisense | 3724 |
| 205890_s_at | 814 | 279 | 578 | AATCTCTTACTCAACGAACACATCT | Antisense | 3725 |
| 205890_s_at | 573 | 349 | 598 | CATCTTCTGATGATTTCCCAAAATT | Antisense | 3726 |
| 205890_s_at | 415 | 7 | 678 | ATTGCCCAACTCTATGTTTCTTTGA | Antisense | 3727 |
| 206035_at | 580 | 271 | 1973 | AATGGATTGATTCCTGCCTTTATTC | Antisense | 3728 |
| 206035_at | 531 | 1109 | 2025 | TTTTCTTCACACATCGAATACCCAA | Antisense | 3729 |
| 206035_at | 826 | 1001 | 2068 | TCTGCGTTCTTGTCCAAATTCTGCT | Antisense | 3730 |
| 206035_at | 206 | 291 | 2084 | AATTCTGCTTCATAGTAGCCGTCTC | Antisense | 3731 |
| 206035_at | 672 | 1005 | 2105 | TCTCTGCAGTCTTTTCCAACTAAAT | Antisense | 3732 |
| 206035_at | 458 | 173 | 2165 | ACTAATGTAATTCTCACTTTTCCTT | Antisense | 3733 |
| 206035_at | 354 | 365 | 2181 | CTTTTCCTTTTCCATAATAGTTCAT | Antisense | 3734 |
| 206035_at | 289 | 265 | 2217 | AAGGGTATGTTCGGTTGTTGTCTGT | Antisense | 3735 |
| 206035_at | 145 | 1093 | 2284 | TTTGTATCTAAAACGCATTCCCCTC | Antisense | 3736 |
| 206035_at | 1154 | 911 | 2313 | TGGGTTGTTCAATTATCTCTATATA | Antisense | 3737 |
| 206035_at | 1020 | 1051 | 2334 | TATACGGGTTATACGCACCGGAGGC | Antisense | 3738 |
| 207397_s_at | 907 | 667 | 883 | GATGTGGCTCTAAATCAGCCGGACA | Antisense | 3739 |
| 207397_s_at | 1061 | 437 | 901 | CCGGACATGTGCGTCTACCGAAGAG | Antisense | 3740 |
| 207397_s_at | 851 | 761 | 909 | GTGCGTCTACCGAAGAGGGAGGAAG | Antisense | 3741 |
| 207397_s_at | 484 | 115 | 935 | AGAGAGTGCCTTACACCAAACTGCA | Antisense | 3742 |
| 207397_s_at | 363 | 303 | 951 | CAAACTGCAGCTTAAAGAACTGGAG | Antisense | 3743 |
| 207397_s_at | 501 | 577 | 967 | GAACTGGAGAACGAGTATGCCATTA | Antisense | 3744 |
| 207397_s_at | 1075 | 995 | 1027 | TCGGCTGCTACGAACCTATCTGAGA | Antisense | 3745 |
| 207397_s_at | 217 | 963 | 1032 | TGCTACGAACCTATCTGAGAGACAA | Antisense | 3746 |
| 207397_s_at | 396 | 641 | 1048 | GAGAGACAAGTGACCATTTGGTTTC | Antisense | 3747 |
| 207397_s_at | 339 | 195 | 1091 | ACAAGAAAATTGTCTCCAAGCTCAA | Antisense | 3748 |
| 207397_s_at | 662 | 753 | 1102 | GTCTCCAAGCTCAAAGATACTGTCT | Antisense | 3749 |
| 207651_at | 757 | 1147 | 828 | TTGCCTTGTAATTCGACAGCTCTAC | Antisense | 3750 |
| 207651_at | 1133 | 175 | 906 | ACTTTTAGTGACCACGGGCTACATC | Antisense | 3751 |
| 207651_at | 103 | 795 | 921 | GGGCTACATCATATGCTTTGTTCCT | Antisense | 3752 |
| 207651_at | 23 | 31 | 968 | ATACCCTCAGCCAGACAGAAGTCAT | Antisense | 3753 |
| 207651_at | 997 | 563 | 985 | GAAGTCATAACTGATTGCTCAACCA | Antisense | 3754 |
| 207651_at | 1084 | 961 | 1000 | TGCTCAACCAGGATTTCACTCTTCA | Antisense | 3755 |
| 207651_at | 572 | 245 | 1025 | AAGCCAAAGAGGCTACACTGCTCCT | Antisense | 3756 |
| 207651_at | 1026 | 961 | 1066 | TGCTTTGATCCTATCCTGTACTATC | Antisense | 3757 |
| 207651_at | 257 | 211 | 1099 | AAAGCATTCCGCTCAAAGGTCACTG | Antisense | 3758 |
| 207651_at | 1005 | 1027 | 1118 | TCACTGAGACTTTTGCCTCACCTAA | Antisense | 3759 |
| 207651_at | 800 | 959 | 1208 | TGCTACCAATTCTGGCCTTACTGGA | Antisense | 3760 |
| 208296_x_at | 1080 | 1151 | 1417 | TTGAGTTCTCCTTTTAAGTACCAAT | Antisense | 3761 |
| 208296_x_at | 428 | 811 | 1469 | GGTGTGTCATTGCCTTGAAATGCTT | Antisense | 3762 |
| 208296_x_at | 537 | 595 | 1485 | GAAATGCTTGCTTAGGGCTTCTTTT | Antisense | 3763 |
| 208296_x_at | 564 | 1073 | 1497 | TAGGGCTTCTTTTATGTTATCTTAA | Antisense | 3764 |
| 208296_x_at | 1109 | 261 | 1523 | AAGTGCTGGTGAATTTTCCATTTTT | Antisense | 3765 |
| 208296_x_at | 683 | 1145 | 1538 | TTCCATTTTTTACATCCATTTCACA | Antisense | 3766 |
| 208296_x_at | 857 | 593 | 1627 | GAAAGGTAACAATCTTCATTCTACA | Antisense | 3767 |
| 208296_x_at | 713 | 275 | 1727 | AATGCGTGCGTTTCAGTGTTTAAGA | Antisense | 3768 |
| 208296_x_at | 1046 | 297 | 1843 | CAAGCAGTTTGTGTGGTGTTTCTAT | Antisense | 3769 |
| 208296_x_at | 1051 | 201 | 1928 | AAATCTGCTGGCCAGCTATGTCCTC | Antisense | 3770 |
| 208296_x_at | 1016 | 1071 | 1953 | TAGGAAATGACAGACCCAACCACCA | Antisense | 3771 |
| 208478_s_at | 18 | 89 | 95 | AGGGTTTCATCCAGGATCGAGCAGG | Antisense | 3772 |
| 208478_s_at | 361 | 1101 | 99 | TTTCATCCAGGATCGAGCAGGGCGA | Antisense | 3773 |
| 208478_s_at | 292 | 839 | 108 | GGATCGAGCAGGGCGAATGGGGGGG | Antisense | 3774 |
| 208478_s_at | 888 | 527 | 115 | GCAGGGCGAATGGGGGGGGAGGCAC | Antisense | 3775 |
| 208478_s_at | 717 | 447 | 163 | CCTCAGGATGCGTCCACCAAGAAGC | Antisense | 3776 |
| 208478_s_at | 664 | 967 | 171 | TGCGTCCACCAAGAAGCTGAGCGAG | Antisense | 3777 |
| 208478_s_at | 507 | 501 | 186 | GCTGAGCGAGTGTCTCAAGCGCATC | Antisense | 3778 |
| 208478_s_at | 709 | 741 | 197 | GTCTCAAGCGCATCGGGGACGAACT | Antisense | 3779 |
| 208478_s_at | 145 | 489 | 204 | GCGCATCGGGGACGAACTGGACAGT | Antisense | 3780 |
| 208478_s_at | 852 | 577 | 217 | GAACTGGACAGTAACATGGAGCTGC | Antisense | 3781 |
| 208478_s_at | 556 | 333 | 225 | CAGTAACATGGAGCTGCAGAGGATG | Antisense | 3782 |
| 208799_at | 1121 | 407 | 511 | CTGGCCTCTACTACGTGGACAGTGA | Antisense | 3783 |
| 208799_at | 55 | 1075 | 568 | TAGGTTCTGGCTCTGTGTATGCATA | Antisense | 3784 |
| 208799_at | 509 | 903 | 601 | TGGATCGGGGCTATTCCTATGACCT | Antisense | 3785 |
| 208799_at | 757 | 849 | 632 | GGAGCAGGCCTATGATCTGGCCCGT | Antisense | 3786 |
| 208799_at | 857 | 81 | 695 | AGGAGGTGCAGTCAACCTCTACCAC | Antisense | 3787 |
| 208799_at | 941 | 843 | 725 | GGAGGATGGCTGGATCCGAGTCTCC | Antisense | 3788 |
| 208799_at | 569 | 941 | 773 | TGAGAAGTATAGTGGCTCTACCCCC | Antisense | 3789 |
| 208799_at | 649 | 777 | 823 | GTGTTTCTTGGGGTGACTGTCATTG | Antisense | 3790 |
| 208799_at | 694 | 1033 | 849 | TAATACGGACACAGTGACCCATCCT | Antisense | 3791 |
| 208799_at | 69 | 563 | 891 | GAAGGGCCTTCAATTGTATCAGTAC | Antisense | 3792 |
| 208799_at | 511 | 1041 | 923 | TAAGCTCTGGCACATTGACCTCTAT | Antisense | 3793 |
| 208885_at | 165 | 565 | 2602 | GAAGTAAGCCTCATCATCAGAGCCT | Antisense | 3794 |
| 208885_at | 314 | 37 | 2617 | ATCAGAGCCTTTCCTCAAAACTGGA | Antisense | 3795 |
| 208885_at | 1091 | 843 | 2639 | GGAGTCCCAAATGTCATCAGGTTTT | Antisense | 3796 |
| 208885_at | 551 | 159 | 2685 | ACCCCTCTGCTTTTAACTCTAGAAT | Antisense | 3797 |
| 208885_at | 103 | 447 | 2748 | CCTCTAGAGCCTTCAGCCTTAATGG | Antisense | 3798 |
| 208885_at | 864 | 281 | 2806 | AATCAAGCCACTCGGCAGGCATGGA | Antisense | 3799 |
| 208885_at | 289 | 1041 | 2839 | TAAGCATCCTTAGGGTTCTGCCTCT | Antisense | 3800 |
| 208885_at | 979 | 389 | 2862 | CTCCAGGCATTAGCCCTCACATTAG | Antisense | 3801 |
| 208885_at | 345 | 1091 | 2923 | TTTGGAGGCAATCCTACCTTGCTTT | Antisense | 3802 |
| 208885_at | 952 | 919 | 3057 | TGTGCCAATCAATAGCACCCCTACT | Antisense | 3803 |
| 208885_at | 472 | 193 | 3091 | ACACACCTAGCCAGCTGTCAAGGGC | Antisense | 3804 |
| 209140_x_at | 320 | 621 | 816 | GACCTGGCAGCGGGATGGCGAGGAC | Antisense | 3805 |
| 209140_x_at | 595 | 433 | 1017 | CCCCATCGTGGGCATTGTTGCTGGC | Antisense | 3806 |
| 209140_x_at | 268 | 885 | 1044 | GGCTGTCCTAGCAGTTGTGGTCATC | Antisense | 3807 |
| 209140_x_at | 907 | 403 | 1073 | CTGTGGTCGCTGCTGTGATGTGTAG | Antisense | 3808 |
| 209140_x_at | 786 | 491 | 1148 | GCGACAGTGCCCAGGGCTCTGATGT | Antisense | 3809 |
| 209140_x_at | 667 | 637 | 1196 | GAGACAGCTGTCTTGTGAGGGACTG | Antisense | 3810 |
| 209140_x_at | 954 | 643 | 1220 | GAGATGCAGGATTTCTTCACGCCTC | Antisense | 3811 |
| 209140_x_at | 233 | 769 | 1251 | GTGACTTCAAGAGCCTCTGGCATCT | Antisense | 3812 |
| 209140_x_at | 568 | 523 | 1270 | GCATCTCTTTCTGCAAAGGCACCTG | Antisense | 3813 |
| 209140_x_at | 146 | 269 | 1285 | AAGGCACCTGAATGTGTCTGCGTCC | Antisense | 3814 |
| 209140_x_at | 305 | 753 | 1300 | GTCTGCGTCCCTGTTAGCATAATGT | Antisense | 3815 |
| 209567_at | 536 | 435 | 1146 | CCGCCTGGCTTGGGTGGCAAGAGAA | Antisense | 3816 |
| 209567_at | 1044 | 123 | 1167 | AGAAAAGGAGGACAGCGCCCAGGAG | Antisense | 3817 |
| 209567_at | 298 | 1099 | 1212 | TTTCTAACTGTCGGACCCGTCTGTA | Antisense | 3818 |
| 209567_at | 270 | 619 | 1225 | GACCCGTCTGTAAACCAAGGACTAT | Antisense | 3819 |
| 209567_at | 53 | 705 | 1266 | GTTCTAGGCAATTATACGGGGACTC | Antisense | 3820 |
| 209567_at | 487 | 483 | 1302 | GCCGCTGCCTTCATTGAGTTTAAAG | Antisense | 3821 |
| 209567_at | 382 | 693 | 1333 | GATTGCCCTTCCGTCAAGAAAGTAT | Antisense | 3822 |
| 209567_at | 181 | 1111 | 1386 | TTTTCCCACAACCGAGACTTTGGAG | Antisense | 3823 |
| 209567_at | 754 | 695 | 1531 | GTTGGCCATATTTCAGAGACTTAGA | Antisense | 3824 |
| 209567_at | 167 | 695 | 1554 | GATTGACGTATATGTTTCTGCATTA | Antisense | 3825 |
| 209567_at | 519 | 721 | 1597 | GTATCAGAGCGGGAGTGCGGGGGAG | Antisense | 3826 |
| 209606_at | 595 | 493 | 1164 | GAATTGCAAAACTGACATCCCATTT | Antisense | 3827 |
| 209606_at | 140 | 169 | 1174 | ACTGACATCCCATTTCACAGCAATA | Antisense | 3828 |
| 209606_at | 1008 | 309 | 1189 | CACAGCAATAGTGACCTTTATTTAA | Antisense | 3829 |
| 209606_at | 778 | 697 | 1217 | GTTGTGTTATAGTTTATGCTTCTTA | Antisense | 3830 |
| 209606_at | 422 | 1099 | 1249 | TTTCAACCTAAACAGCCAATTTCTA | Antisense | 3831 |
| 209606_at | 735 | 189 | 1260 | ACAGCCAATTTCTAAGCAGACAGGA | Antisense | 3832 |
| 209606_at | 240 | 1087 | 1369 | TATTTTCTAGATTATCCCTGTGAAT | Antisense | 3833 |
| 209606_at | 292 | 201 | 1485 | AAATCTTAGGTTTGCTTATGCCCAG | Antisense | 3834 |
| 209606_at | 775 | 499 | 1590 | GCTGTTTCTCACATCTATAGTGACA | Antisense | 3835 |
| 209606_at | 195 | 25 | 1639 | ATAGGAGGGGTTAAGGTTCATGAGA | Antisense | 3836 |
| 209606_at | 790 | 823 | 1679 | GGTCTGAGATGGGTGCTGCAAAGAT | Antisense | 3837 |
| 209774_x_at | 524 | 115 | 477 | AGAGAGACACAGCTGCAGAGGCCAC | Antisense | 3838 |
| 209774_x_at | 464 | 157 | 500 | ACCTGGATTGCGCCTAATGTGTTTG | Antisense | 3839 |
| 209774_x_at | 864 | 933 | 627 | TGATTGAATCTACTTGCACACTCTC | Antisense | 3840 |
| 209774_x_at | 256 | 541 | 642 | GCACACTCTCCCATTATATTTATTG | Antisense | 3841 |
| 209774_x_at | 1059 | 239 | 681 | AACCCAAGTTAGTTCAATCCTGATT | Antisense | 3842 |
| 209774_x_at | 486 | 561 | 725 | GAAGGTTTGCAGATATTCTCTAGTC | Antisense | 3843 |
| 209774_x_at | 232 | 607 | 773 | GACATATCACATGTCAGCCACTGTG | Antisense | 3844 |
| 209774_x_at | 1021 | 71 | 822 | ATGGCCAGTAAGATCAATGTGACGG | Antisense | 3845 |
| 209774_x_at | 628 | 919 | 839 | TGTGACGGCAGGGAAATGTATGTGT | Antisense | 3846 |
| 209774_x_at | 281 | 337 | 920 | CAGTGTGTGGTCAACATTTCTCATG | Antisense | 3847 |
| 209774_x_at | 889 | 61 | 1023 | ATGTTAATTATGCAGTGTTTCCCTC | Antisense | 3848 |
| 209849_s_at | 136 | 1059 | 710 | TACTGGCACAAGTTTATCTTCTTCC | Antisense | 3849 |
| 209849_s_at | 1139 | 41 | 725 | ATCTTCTTCCAGATTTCCTTTCAGA | Antisense | 3850 |
| 209849_s_at | 485 | 961 | 786 | TGCTTTTCCATTTCGTCATGACCTA | Antisense | 3851 |
| 209849_s_at | 608 | 401 | 817 | CTGTCTCTTCGTACTCGGTTATTAA | Antisense | 3852 |
| 209849_s_at | 895 | 451 | 938 | CCTTGCTTGTTCCTGCATTAGGGGA | Antisense | 3853 |
| 209849_s_at | 308 | 861 | 960 | GGAAAGTTGGGGACATGCTGCTACA | Antisense | 3854 |
| 209849_s_at | 111 | 503 | 976 | GCTGCTACAATACGGCTAATCTTTC | Antisense | 3855 |
| 209849_s_at | 949 | 543 | 1024 | GCAACATTGTACAAGTCACCCAGCC | Antisense | 3856 |
| 209849_s_at | 1123 | 743 | 1038 | GTCACCCAGCCAGAAGGAATGCACA | Antisense | 3857 |
| 209849_s_at | 862 | 683 | 1096 | GATACTGTTGTTACTTCTGCATGTT | Antisense | 3858 |
| 209849_s_at | 660 | 625 | 1143 | GAGCACCCGGAAACGGTCACGAGAC | Antisense | 3859 |
| 209894_at | 1074 | 877 | 4425 | GGCATAGGAACAGTTTTCTCTTCAT | Antisense | 3860 |
| 209894_at | 1099 | 289 | 4484 | AATTCAGTATTTGTCATTATGCAGT | Antisense | 3861 |
| 209894_at | 37 | 31 | 4515 | ATACCTACATAAGTCTATTCCATTA | Antisense | 3862 |
| 209894_at | 605 | 1111 | 4545 | TTTTGCGCTTGGCATATTTATTCCT | Antisense | 3863 |
| 209894_at | 841 | 141 | 4666 | AGCTGGGTATACCATTATTTCAAGT | Antisense | 3864 |
| 209894_at | 967 | 581 | 4761 | GAACACATTTTCTATAGCCCTTAAT | Antisense | 3865 |
| 209894_at | 1019 | 1081 | 4773 | TATAGCCCTTAATTTAGTTGTGTTA | Antisense | 3866 |
| 209894_at | 156 | 819 | 4807 | GGTGATGTATCAACAGCTTTTTTTT | Antisense | 3867 |
| 209894_at | 378 | 725 | 4863 | GTATGAATTGAGCTTTTTGCCCACA | Antisense | 3868 |
| 209894_at | 641 | 1111 | 4877 | TTTTGCCCACAGATTCTTGATTTGT | Antisense | 3869 |
| 209894_at | 899 | 357 | 4892 | CTTGATTTGTAGTTGTTTGGCAGGA | Antisense | 3870 |
| 210260_s_at | 1031 | 693 | 1244 | GATTGAGTCATCGACATTCAGGATT | Antisense | 3871 |
| 210260_s_at | 234 | 1 | 1263 | AGGATTTAAGTCTGAGGTAGTCAAC | Antisense | 3872 |
| 210260_s_at | 946 | 219 | 1300 | AAAAATGGCTTATCTGAAATCAGTA | Antisense | 3873 |
| 210260_s_at | 544 | 55 | 1376 | ATGCTTCTGGAATTGAGTTCTCCTT | Antisense | 3874 |
| 210260_s_at | 142 | 647 | 1390 | GAGTTCTCCTTTTAAGTACCAATGA | Antisense | 3875 |
| 210260_s_at | 855 | 923 | 1434 | TGTAATGGTGTGTCATTGCCTTGAA | Antisense | 3876 |
| 210260_s_at | 897 | 779 | 1443 | GTGTCATTGCCTTGAAATGCTTGCT | Antisense | 3877 |
| 210260_s_at | 276 | 949 | 1455 | TGAAATGCTTGCTTAGGGCTTCTTT | Antisense | 3878 |
| 210260_s_at | 86 | 223 | 1546 | AAAAGTCTAGATTGGTCTTGATATT | Antisense | 3879 |
| 210260_s_at | 748 | 293 | 1607 | CAATCTTCATTCTACAGATGAACTC | Antisense | 3880 |
| 210260_s_at | 833 | 493 | 1701 | GCGTGCGTTTCAGTGTTTAAGAAGG | Antisense | 3881 |
| 210538_s_at | 297 | 593 | 2493 | GAAAGTGTCCTATTTGTAGGAGTAC | Antisense | 3882 |
| 210538_s_at | 59 | 847 | 2511 | GGAGTACAATCAAGGGTACAGTTCG | Antisense | 3883 |
| 210538_s_at | 551 | 735 | 2526 | GTACAGTTCGTACATTTCTTTCATG | Antisense | 3884 |
| 210538_s_at | 135 | 27 | 2741 | ATAGGCTTTTGTTCTTATGAACGAA | Antisense | 3885 |
| 210538_s_at | 909 | 253 | 2767 | AAGAGGTAGCACTACAAACACAATA | Antisense | 3886 |
| 210538_s_at | 97 | 287 | 2871 | AATATTTTGGCATTGTACTAATACC | Antisense | 3887 |
| 210538_s_at | 727 | 873 | 2932 | GGCAGCATACTGAGACCCTGCCTTT | Antisense | 3888 |
| 210538_s_at | 456 | 221 | 2978 | AAAACACCAGGGACACATTTCTCTG | Antisense | 3889 |
| 210538_s_at | 512 | 337 | 2985 | CAGGGACACATTTCTCTGTCTTTTT | Antisense | 3890 |
| 210538_s_at | 821 | 1091 | 3008 | TTTGATCAGTGTCCTATACATCGAA | Antisense | 3891 |
| 210538_s_at | 935 | 759 | 3018 | GTCCTATACATCGAAGGTGTGCATA | Antisense | 3892 |
| 211529_x_at | 456 | 321 | 379 | CACCACCCTGTCTTTGACTATGAGG | Antisense | 3893 |
| 211529_x_at | 93 | 611 | 394 | GACTATGAGGCCACCCTGAGGTGCT | Antisense | 3894 |
| 211529_x_at | 47 | 1061 | 433 | TACCCTGCGGAGATCATACTGACCT | Antisense | 3895 |
| 211529_x_at | 349 | 617 | 487 | GACGTGGAGCTCGTGGAGACCAGGC | Antisense | 3896 |
| 211529_x_at | 896 | 643 | 573 | GAGATACACGTGCCATGTGCAGCAT | Antisense | 3897 |
| 211529_x_at | 145 | 643 | 624 | GAGATGGAAGCAGTCTTCCCTGCCC | Antisense | 3898 |
| 211529_x_at | 281 | 1157 | 683 | TTGTCCTTGCAGCTGTAGTCACTGG | Antisense | 3899 |
| 211529_x_at | 1098 | 137 | 763 | AGCTACTCTCAGGCTGCAAGCAGTG | Antisense | 3900 |
| 211529_x_at | 474 | 335 | 789 | CAGTGCCCAGGGCTCTAATGTGTCT | Antisense | 3901 |
| 211529_x_at | 976 | 779 | 808 | GTGTCTCTCACGGCTTGTAAATTGT | Antisense | 3902 |
| 211529_x_at | 1010 | 187 | 836 | ACAGCTGCCCTGTGTGGGACTGAGT | Antisense | 3903 |
| 211595_s_at | 673 | 193 | 560 | ACACAGATCCAGGTAGTCTCTGCTA | Antisense | 3904 |
| 211595_s_at | 320 | 753 | 575 | GTCTCTGCTAGTAATGAGCCCCTTG | Antisense | 3905 |
| 211595_s_at | 492 | 131 | 658 | AGCACAGACAGCAGGCATAGCCGCA | Antisense | 3906 |
| 211595_s_at | 197 | 145 | 676 | AGCCGCAGCGGCGAGAGCTAAACAA | Antisense | 3907 |
| 211595_s_at | 329 | 303 | 698 | CAAAAGGGCGTGATCCACATCCGAG | Antisense | 3908 |
| 211595_s_at | 1006 | 1157 | 752 | TTGTCTGCCATGCACGGACTGATCA | Antisense | 3909 |
| 211595_s_at | 331 | 855 | 767 | GGACTGATCATGGGCGGCCTGGAAG | Antisense | 3910 |
| 211595_s_at | 517 | 377 | 899 | CTCAGCTCCAGTGGGACCTTGTAAA | Antisense | 3911 |
| 211595_s_at | 203 | 853 | 912 | GGACCTTGTAAAATGCTCCCTGTCA | Antisense | 3912 |
| 211595_s_at | 996 | 989 | 928 | TCCCTGTCAGAGCTCTCCAGAATAT | Antisense | 3913 |
| 211595_s_at | 549 | 815 | 1036 | GGTGTGCCCCAGAAGTAAGCTTTGC | Antisense | 3914 |
| 211603_s_at | 522 | 821 | 1524 | GGTGCTGCCCTGTGTACATATAAAT | Antisense | 3915 |
| 211603_s_at | 740 | 817 | 1555 | GGTGTTGGGGAAACCTTCATCTGAA | Antisense | 3916 |
| 211603_s_at | 193 | 121 | 1634 | AGACTCTGAGCTGCTCACCGGAGTC | Antisense | 3917 |
| 211603_s_at | 960 | 501 | 1643 | GCTGCTCACCGGAGTCATTGGGAAG | Antisense | 3918 |
| 211603_s_at | 23 | 371 | 1690 | CTAGAGTCTCAGAAACTCCCCTGGG | Antisense | 3919 |
| 211603_s_at | 491 | 843 | 1732 | GGAGGAATTCAGCTCAGCTTCTTCC | Antisense | 3920 |
| 211603_s_at | 664 | 195 | 1797 | ACAAGAGTTTGTTCTGTTCTGGGGG | Antisense | 3921 |
| 211603_s_at | 444 | 995 | 1814 | TCTGGGGGACAGAGAAGGCGCTTCC | Antisense | 3922 |
| 211603_s_at | 991 | 515 | 1833 | GCTTCCCAACTTCATACTGGCAGGA | Antisense | 3923 |
| 211603_s_at | 540 | 819 | 1859 | GGTGAGGAGGTTCACTGAGCTCCCC | Antisense | 3924 |
| 211603_s_at | 718 | 421 | 1890 | CCCACTGCGGGGAGACAGAAGCCTG | Antisense | 3925 |
| 211700_s_at | 28 | 461 | 2132 | CCAGCACGGGCTTCAGTAGTGGACC | Antisense | 3926 |
| 211700_s_at | 703 | 853 | 2152 | GGACCCAGTTCTATTGTTGGCTTCA | Antisense | 3927 |
| 211700_s_at | 182 | 517 | 2171 | GCTTCAGCGGTGGACCAAGCACTGG | Antisense | 3928 |
| 211700_s_at | 495 | 467 | 2185 | CCAAGCACTGGTGTTGGCTTCTGCA | Antisense | 3929 |
| 211700_s_at | 805 | 91 | 2209 | AGTGGACCAAGCACCAGTGGCTTCA | Antisense | 3930 |
| 211700_s_at | 183 | 517 | 2228 | GCTTCAGCGGTGGACCGAGCACAGG | Antisense | 3931 |
| 211700_s_at | 777 | 435 | 2242 | CCGAGCACAGGAGCTGGCTTCGGCG | Antisense | 3932 |
| 211700_s_at | 1099 | 357 | 2259 | CTTCGGCGGTGGACCAAACACTGGT | Antisense | 3933 |
| 211700_s_at | 995 | 157 | 2301 | ACCGAGCACCAGTGCTGGCTTTGGC | Antisense | 3934 |
| 211700_s_at | 396 | 967 | 2313 | TGCTGGCTTTGGCAGTGGAGCCGCC | Antisense | 3935 |
| 211700_s_at | 1091 | 789 | 2351 | GTGGCTTCTCGTATGGCTAGTGAGG | Antisense | 3936 |
| 212349_at | 697 | 903 | 4080 | TGGAGTGTGTTCCTGAAGAGCAGCC | Antisense | 3937 |
| 212349_at | 103 | 663 | 4125 | GAGGCAGGCATAGGCAGGGAACCGA | Antisense | 3938 |
| 212349_at | 156 | 799 | 4141 | GGGAACCGAGCAGCAGGTCAGAGCA | Antisense | 3939 |
| 212349_at | 1027 | 73 | 4165 | AGGCGAGCTGACATTCTGCAGCCTG | Antisense | 3940 |
| 212349_at | 733 | 481 | 4185 | GCCTGGACGGCCATGGCAGGAAGCT | Antisense | 3941 |
| 212349_at | 913 | 245 | 4230 | AAGCCTCCTAGGGTTCTGAGCAGAA | Antisense | 3942 |
| 212349_at | 1123 | 813 | 4257 | GGGGCATGAGCTGATTCACATTCTG | Antisense | 3943 |
| 212349_at | 955 | 179 | 4274 | ACATTCTGAAGGACCTCTCTAGCTG | Antisense | 3944 |
| 212349_at | 236 | 211 | 4333 | AAAGCAGAGAGACCAGTGCAGGGCT | Antisense | 3945 |
| 212349_at | 790 | 699 | 4367 | GTTGCAGGCGAGAGACTGGGGTGCT | Antisense | 3946 |
| 212349_at | 173 | 1005 | 4425 | TCTCCCAAGAGACAAAGGCCATTGC | Antisense | 3947 |
| 212654_at | 304 | 815 | 804 | GGTGGCCGAGAGTAAATGTGGGGAC | Antisense | 3948 |
| 212654_at | 339 | 737 | 815 | GTAAATGTGGGGACCTAGAGGAGGA | Antisense | 3949 |
| 212654_at | 180 | 903 | 872 | TGGAGGCCCAGGCGGACAAGTATTC | Antisense | 3950 |
| 212654_at | 145 | 341 | 880 | CAGGCGGACAAGTATTCCACCAAAG | Antisense | 3951 |
| 212654_at | 339 | 673 | 1024 | GATGAAGTCTATGCCCAGAAGATGA | Antisense | 3952 |
| 212654_at | 447 | 77 | 1067 | AGGAACTGGACAACGCACTCAATGA | Antisense | 3953 |
| 212654_at | 592 | 599 | 1075 | GACAACGCACTCAATGACATCACCT | Antisense | 3954 |
| 212654_at | 541 | 981 | 1149 | TCCTTTCCATTCTCTCTATGGGGAG | Antisense | 3955 |
| 212654_at | 13 | 661 | 1186 | GAGGAGCAGAAATTGCCAACATTGC | Antisense | 3956 |
| 212654_at | 183 | 1147 | 1198 | TTGCCAACATTGCACAGCCAGGCTG | Antisense | 3957 |
| 212654_at | 208 | 405 | 1220 | CTGGGAGCAGCCTAGGGAGAGCCCC | Antisense | 3958 |
| 212671_s_at | 689 | 149 | 439 | ACCAATGAGGTTCCTGAGGTCACAG | Antisense | 3959 |
| 212671_s_at | 593 | 59 | 443 | ATGAGGTTCCTGAGGTCACAGTGTT | Antisense | 3960 |
| 212671_s_at | 710 | 83 | 446 | AGGTTCCTGAGGTCACAGTGTTTTC | Antisense | 3961 |
| 212671_s_at | 604 | 983 | 450 | TCCTGAGGTCACAGTGTTTTCCAAG | Antisense | 3962 |
| 212671_s_at | 458 | 749 | 512 | GTCTTGTGGACAACATCTTTCCTCC | Antisense | 3963 |
| 212671_s_at | 615 | 383 | 604 | CTCTCCAAGAGTGATCATTCCTTCT | Antisense | 3964 |
| 212671_s_at | 807 | 467 | 608 | CCAAGAGTGATCATTCCTTCTTCAA | Antisense | 3965 |
| 212671_s_at | 232 | 113 | 611 | AGAGTGATCATTCCTTCTTCAAGAT | Antisense | 3966 |
| 212671_s_at | 793 | 93 | 613 | AGTGATCATTCCTTCTTCAAGATCA | Antisense | 3967 |
| 212671_s_at | 731 | 671 | 664 | GATGAGATTTATGACTGCAAGGTGG | Antisense | 3968 |
| 212671_s_at | 573 | 443 | 709 | CCTCTTCTGAAACACTGGGAGCCTG | Antisense | 3969 |
| 212877_at | 1091 | 657 | 411 | GAGGAAAATGAAGCTCGGGCTGGTT | Antisense | 3970 |
| 212877_at | 973 | 571 | 420 | GAAGCTCGGGCTGGTTAACTGACTT | Antisense | 3971 |
| 212877_at | 178 | 505 | 423 | GCTCGGGCTGGTTAACTGACTTGCT | Antisense | 3972 |
| 212877_at | 519 | 407 | 426 | CGGGCTGGTTAACTGACTTGCTCAG | Antisense | 3973 |
| 212877_at | 244 | 497 | 429 | GCTGGTTAACTGACTTGCTCAGCGT | Antisense | 3974 |
| 212877_at | 613 | 417 | 543 | CGCTGTGTCTAGCAGCCTCTAGGAT | Antisense | 3975 |
| 212877_at | 800 | 923 | 548 | TGTCTAGCAGCCTCTAGGATCTTGT | Antisense | 3976 |
| 212877_at | 607 | 441 | 637 | CCTGATTTCTGTATACATGTAGCTT | Antisense | 3977 |
| 212877_at | 713 | 927 | 646 | TGTATACATGTAGCTTTGCCAGATA | Antisense | 3978 |
| 212877_at | 752 | 137 | 657 | AGCTTTGCCAGATATGTACTTAGTA | Antisense | 3979 |
| 212877_at | 1159 | 1083 | 692 | TATTAATAAAATCCATTTACTGTGT | Antisense | 3980 |
| 213008_at | 771 | 1053 | 1275 | TACATTTTTCCACGAGCTGGTGCAG | Antisense | 3981 |
| 213008_at | 433 | 603 | 1299 | GACAGCTCTGCCATCAGGCAGCTGT | Antisense | 3982 |
| 213008_at | 884 | 189 | 1369 | ACAGCCCTTGTCAGATATTATCTCC | Antisense | 3983 |
| 213008_at | 541 | 1003 | 1389 | TCTCCAGGTGTGTCAGAGCTCCGGA | Antisense | 3984 |
| 213008_at | 931 | 951 | 1442 | TGAAGCTGTCTGGTTCTCATCTGAC | Antisense | 3985 |
| 213008_at | 594 | 465 | 1559 | CCACAGCCATGGCCAGAGTTCTTCG | Antisense | 3986 |
| 213008_at | 1106 | 447 | 1600 | CCTAACCTCATCTTTGCCATAGAAC | Antisense | 3987 |
| 213008_at | 132 | 587 | 1630 | GAAAAATTTCTCATCCACCTTTCTA | Antisense | 3988 |
| 213008_at | 982 | 957 | 1676 | TGCAGCACATGAAGCTCAGCACCTC | Antisense | 3989 |
| 213008_at | 242 | 537 | 1694 | GCACCTCACGAGACTTCAAGATCAA | Antisense | 3990 |
| 213008_at | 1091 | 653 | 1771 | GAGGGCACTGCATCAGAGCATGGGG | Antisense | 3991 |
| 213539_at | 163 | 799 | 265 | GGGAACACTGCTCTCAGACATTACA | Antisense | 3992 |
| 213539_at | 711 | 613 | 291 | GACTGGACCTGGGAAAACGCATCCT | Antisense | 3993 |
| 213539_at | 701 | 417 | 308 | CGCATCCTGGACCCACGAGGAATAT | Antisense | 3994 |
| 213539_at | 383 | 553 | 368 | GAATCTACCGTGCAAGTTCATTATC | Antisense | 3995 |
| 213539_at | 835 | 781 | 408 | GTGTGGAGCTGGATCCAGCCACCGT | Antisense | 3996 |
| 213539_at | 610 | 119 | 507 | AGACTGGAAGGCTGTCTGGGGCTGC | Antisense | 3997 |
| 213539_at | 108 | 971 | 529 | TGCCGACACACAAGCTCTGTTGAGG | Antisense | 3998 |
| 213539_at | 964 | 867 | 552 | GGAATGACCAGGTCTATCAGCCCCT | Antisense | 3999 |
| 213539_at | 659 | 145 | 570 | AGCCCCTCCGAGATCGAGATGATGC | Antisense | 4000 |
| 213539_at | 1113 | 961 | 592 | TGCTCAGTACAGCCACCTTGGAGGA | Antisense | 4001 |
| 213539_at | 892 | 569 | 659 | GAAGCAGCCATTACCAACTGTACCT | Antisense | 4002 |
| 214901_at | 123 | 415 | 1597 | CGAAGCCGGCGGCGTGAACAATCCT | Antisense | 4003 |
| 214901_at | 645 | 339 | 1626 | CAGGAACTCACACCTGGTTCAGCAT | Antisense | 4004 |
| 214901_at | 712 | 525 | 1678 | GCAGGCGGAGCAAAGGCAGGGCAGC | Antisense | 4005 |
| 214901_at | 919 | 865 | 1779 | GGAAGAGCCTTCTGTGGGTGCTTCC | Antisense | 4006 |
| 214901_at | 777 | 1019 | 1928 | TCATGGGTGACTTCTGACTTTCTAA | Antisense | 4007 |
| 214901_at | 234 | 359 | 1963 | CTTCCCAAGCACCCGAGGTTGGTTG | Antisense | 4008 |
| 214901_at | 549 | 833 | 1983 | GGTTGGTCCCAAATCTATCAAACTC | Antisense | 4009 |
| 214901_at | 848 | 487 | 2011 | GCCCTCTTTAGCGACATATTTTGTG | Antisense | 4010 |
| 214901_at | 724 | 1157 | 2031 | TTGTGACATTCCTTCCATTACACCA | Antisense | 4011 |
| 214901_at | 1017 | 31 | 2075 | ATAACCTACCCACCTGTGTAATGTC | Antisense | 4012 |
| 214901_at | 881 | 281 | 2106 | AATCAATATGCGGCCCCATTTTGTA | Antisense | 4013 |
| 216438_s_at | 1086 | 185 | 150 | ACAGAGACGCAAGAGAAAAATCCAC | Antisense | 4014 |
| 216438_s_at | 546 | 277 | 168 | AATCCACTGCCTTCCAAAGAAACGA | Antisense | 4015 |
| 216438_s_at | 138 | 315 | 172 | CACTGCCTTCCAAAGAAACGATTGA | Antisense | 4016 |
| 216438_s_at | 670 | 849 | 200 | GGAGAAGCAAGCAGGCGAATCGTAA | Antisense | 4017 |
| 216438_s_at | 49 | 135 | 209 | AGCAGGCGAATCGTAATGAGGCGTG | Antisense | 4018 |
| 216438_s_at | 556 | 957 | 245 | TGCACTGTACATTCCACAAGCATTG | Antisense | 4019 |
| 216438_s_at | 569 | 167 | 248 | ACTGTACATTCCACAAGCATTGCCT | Antisense | 4020 |
| 216438_s_at | 9 | 109 | 308 | AGATGCAAAGAGGTTGGATCAAGTT | Antisense | 4021 |
| 216438_s_at | 178 | 259 | 328 | AAGTTTAAATGACTGTGCTGCCCCT | Antisense | 4022 |
| 216438_s_at | 744 | 591 | 426 | GAAAGAACTTGCATGTTGGTGAAGG | Antisense | 4023 |
| 216438_s_at | 432 | 499 | 516 | GCTGTAATGCAGTTTAATCAGAGTG | Antisense | 4024 |
| 216920_s_at | 416 | 313 | 881 | CACTACTGCTGCAGCTCACAAACAC | Antisense | 4025 |
| 216920_s_at | 585 | 509 | 894 | GCTCACAAACACCTCTGCATATTAC | Antisense | 4026 |
| 216920_s_at | 504 | 1001 | 907 | TCTGCATATTACATGTACCTCCTCC | Antisense | 4027 |
| 216920_s_at | 513 | 783 | 944 | GTGTGGTCTATTTTGCCATCATCAC | Antisense | 4028 |
| 216920_s_at | 940 | 891 | 1115 | GGCCATTTCAGTTCTCATGTGTGTA | Antisense | 4029 |
| 216920_s_at | 978 | 829 | 1161 | GGTTTTCAAACCAGTGGGCACACAG | Antisense | 4030 |
| 216920_s_at | 584 | 989 | 1323 | TCCCTCTCTTAGTGTTCTTTAATCA | Antisense | 4031 |
| 216920_s_at | 126 | 35 | 1349 | ATAACTGCCTGGAAGCCTTTCATTT | Antisense | 4032 |
| 216920_s_at | 170 | 897 | 1358 | TGGAAGCCTTTCATTTTACACGCCC | Antisense | 4033 |
| 216920_s_at | 1055 | 417 | 1378 | CGCCCTGAAGCAGTCTTCTTTGCTA | Antisense | 4034 |
| 216920_s_at | 1041 | 1115 | 1409 | TTATGTGGTGTGTTTTTCCGTAATA | Antisense | 4035 |
| 217456_x_at | 208 | 415 | 1025 | CGACAGTGCCCAGGGGTCTGAGTCT | Antisense | 4036 |
| 217456_x_at | 721 | 213 | 1060 | AAAGCCTGAGACAGCTGCCTTGTGT | Antisense | 4037 |
| 217456_x_at | 1110 | 503 | 1101 | GCTGCCTTGTGTGCGACTGAGATGC | Antisense | 4038 |
| 217456_x_at | 756 | 941 | 1118 | TGAGATGCAGGATTTCCTCACGCCT | Antisense | 4039 |
| 217456_x_at | 505 | 879 | 1167 | GGCTTCTCTTTTTGCAAGGGCCTCT | Antisense | 4040 |
| 217456_x_at | 370 | 999 | 1195 | TCTGTCTGTGTCCCTGTTAGCACAA | Antisense | 4041 |
| 217456_x_at | 687 | 575 | 1320 | GAACCTGGGCAGAGTGCGGCAGTCA | Antisense | 4042 |
| 217456_x_at | 635 | 47 | 1399 | ATCGAAACCATCCTGCTAAGAGGTG | Antisense | 4043 |
| 217456_x_at | 26 | 509 | 1413 | GCTAAGAGGTGAACCGTCTCTACTA | Antisense | 4044 |
| 217456_x_at | 760 | 895 | 1455 | TGGCGCAGAGGCAGGCTGTGATCCA | Antisense | 4045 |
| 217456_x_at | 341 | 773 | 1472 | GTGATCCAGCTACTCAGGAGCGAGC | Antisense | 4046 |
| 217885_at | 1035 | 671 | 1499 | GATGACAATTCAGACCAGGCTCACC | Antisense | 4047 |
| 217885_at | 719 | 821 | 1524 | GGTGCCGTCACTTAGGAATGCTGGA | Antisense | 4048 |
| 217885_at | 899 | 121 | 1578 | AGACATGCCATCTCTAGAACCTTTT | Antisense | 4049 |
| 217885_at | 901 | 491 | 1649 | GCGAGTCCAGAAACATTATTGCCCA | Antisense | 4050 |
| 217885_at | 105 | 81 | 1712 | AGGAGCGCAGGAAGTCACTACCATT | Antisense | 4051 |
| 217885_at | 793 | 705 | 1762 | GTTCATAGGACTTCTGATGTGTTCA | Antisense | 4052 |
| 217885_at | 122 | 645 | 1804 | GAGATCATTATGCTTTGTGCCCTGG | Antisense | 4053 |
| 217885_at | 262 | 871 | 1861 | GGCAAGGGCAGCTTCATTCTAAGCC | Antisense | 4054 |
| 217885_at | 495 | 303 | 1918 | CAACACTAAGGCTCCTCTGTCAGAG | Antisense | 4055 |
| 217885_at | 939 | 111 | 1939 | AGAGGAGGTCGTCTTGTTTTTGCTT | Antisense | 4056 |
| 217885_at | 761 | 1131 | 1962 | TTCATTGCATGACATAACCCTTCCC | Antisense | 4057 |
| 217995_at | 660 | 1055 | 1111 | TACGTCAAAGACCGCTGCTGCAGTA | Antisense | 4058 |
| 217995_at | 560 | 529 | 1130 | GCAGTAGCTGCCCAGTCAGGAATAC | Antisense | 4059 |
| 217995_at | 948 | 509 | 1212 | GCTACACATCATGTCCACTGGTGAC | Antisense | 4060 |
| 217995_at | 28 | 315 | 1227 | CACTGGTGACCGGCTACAACCGTGT | Antisense | 4061 |
| 217995_at | 625 | 195 | 1242 | ACAACCGTGTGATTCTTGCTGAGTT | Antisense | 4062 |
| 217995_at | 896 | 489 | 1318 | GCGCCTTTCCATGTATCTCATGAAA | Antisense | 4063 |
| 217995_at | 615 | 623 | 1398 | GACCAGCGTTTCTGCGCAAGTTGTT | Antisense | 4064 |
| 217995_at | 1 | 1039 | 1439 | TAAGGATGGCTCAGCACTTGCTCAT | Antisense | 4065 |
| 217995_at | 633 | 879 | 1471 | GGCTTCTGGGCCAAAACTGCAGTCA | Antisense | 4066 |
| 217995_at | 1119 | 739 | 1558 | GTAATGGTGACCAAATGCCTCCCTT | Antisense | 4067 |
| 217995_at | 541 | 783 | 1608 | GTGGGCTACTCATGATGGGCTTGAT | Antisense | 4068 |
| 218363_at | 693 | 997 | 2253 | TCTGGACATGAGCCTTTGACCTGGG | Antisense | 4069 |
| 218363_at | 794 | 123 | 2284 | AGAAAGAACCACAAACTCCATCTCC | Antisense | 4070 |
| 218363_at | 648 | 237 | 2297 | AACTCCATCTCCCAATAGAACTTTG | Antisense | 4071 |
| 218363_at | 849 | 1151 | 2319 | TTGAAATTCACTCAGCTTTTCCTTT | Antisense | 4072 |
| 218363_at | 614 | 1111 | 2335 | TTTTCCTTTCATGCTGTTTGTTGCC | Antisense | 4073 |
| 218363_at | 917 | 983 | 2375 | TCCTGCCCCAGAACTGCAAGATTTT | Antisense | 4074 |
| 218363_at | 2 | 517 | 2402 | GCTTCACCCCTTTCTGAGAGTAATG | Antisense | 4075 |
| 218363_at | 961 | 427 | 2456 | CCCTGTATTCTGTGCTTCATCGAAT | Antisense | 4076 |
| 218363_at | 899 | 543 | 2483 | GCAAGACTGACCTCTTTTAAGCATT | Antisense | 4077 |
| 218363_at | 552 | 249 | 2501 | AAGCATTTAATTCACTCCCAGAGTC | Antisense | 4078 |
| 218363_at | 878 | 799 | 2574 | GGGACACTGAGCTTACTTAATACAT | Antisense | 4079 |
| 218807_at | 1032 | 969 | 4366 | TGCCGTCAGCCGAACTTTGTTATGG | Antisense | 4080 |
| 218807_at | 727 | 903 | 4388 | TGGAGGGAGCAGCCTCACACAAGCA | Antisense | 4081 |
| 218807_at | 584 | 587 | 4413 | GAAACACTCCTGTGGATGGTATTGT | Antisense | 4082 |
| 218807_at | 1142 | 1067 | 4456 | TAGTCAATAGACCCTCTCCTTATAA | Antisense | 4083 |
| 218807_at | 255 | 531 | 4529 | GCAGAGGATATTGGAGCCCCTTTTT | Antisense | 4084 |
| 218807_at | 886 | 53 | 4543 | AGCCCCTTTTTGTGACATTACCAAT | Antisense | 4085 |
| 218807_at | 639 | 1063 | 4561 | TACCAATTACATCTTTGTCCACGTT | Antisense | 4086 |
| 218807_at | 420 | 921 | 4576 | TGTCCACGTTTAATACTTTGTTTTG | Antisense | 4087 |
| 218807_at | 77 | 63 | 4656 | ATGTTTTCCATATACTTTGTCTTGC | Antisense | 4088 |
| 218807_at | 752 | 1055 | 4668 | TACTTTGTCTTGCCTGTATGCAGCC | Antisense | 4089 |
| 218807_at | 355 | 723 | 4683 | GTATGCAGCCCTTGTGTAATATGGT | Antisense | 4090 |
| 221265_s_at | 266 | 1025 | 1980 | TCACCTCTAGAATAGCCACCCAAAG | Antisense | 4091 |
| 221265_s_at | 798 | 117 | 2003 | AGACCTTCCTGAGGCTGCCTCAGAA | Antisense | 4092 |
| 221265_s_at | 430 | 447 | 2020 | CCTCAGAAGCACCACTTGCTGTTTT | Antisense | 4093 |
| 221265_s_at | 707 | 565 | 2130 | GAAGATAGCTGCTGTTCCCATGATG | Antisense | 4094 |
| 221265_s_at | 541 | 991 | 2145 | TCCCATGATGGGCACATTTCCTGAG | Antisense | 4095 |
| 221265_s_at | 485 | 167 | 2181 | ACTGATGAGCATAGAGCACCCCTGC | Antisense | 4096 |
| 221265_s_at | 85 | 341 | 2212 | CAGGAAACCTGACCGGCAGGGGCTC | Antisense | 4097 |
| 221265_s_at | 243 | 385 | 2236 | CTCTGGCTTCCTGAAAGCTTCACCT | Antisense | 4098 |
| 221265_s_at | 1056 | 1011 | 2260 | TCTTCCCTCGTTTATATCTCAACTG | Antisense | 4099 |
| 221265_s_at | 858 | 543 | 2319 | GCAACTCAGACTGTCTGGAGCTTGC | Antisense | 4100 |
| 221265_s_at | 242 | 715 | 2368 | GTTTCAGAACTTTCCTTAGGACTTG | Antisense | 4101 |
| 221875_x_at | 921 | 1007 | 526 | TCTACCCTGCGGAGATCACGCTGAC | Antisense | 4102 |
| 221875_x_at | 352 | 863 | 569 | GGAACAGACCCAGGACACAGAGCTT | Antisense | 4103 |
| 221875_x_at | 320 | 329 | 586 | CAGAGCTTGTGGAGACCAGGCCTGC | Antisense | 4104 |
| 221875_x_at | 623 | 73 | 603 | AGGCCTGCAGGGGATGGAACCTTCC | Antisense | 4105 |
| 221875_x_at | 183 | 975 | 625 | TCCAGAAGTGGGCCGCTGTGGTGGT | Antisense | 4106 |
| 221875_x_at | 576 | 323 | 705 | CAGCCCCTCATCCTGAGATGGGAGC | Antisense | 4107 |
| 221875_x_at | 692 | 929 | 776 | TGTTGTCCTTGGAGCTGTGGTCACT | Antisense | 4108 |
| 221875_x_at | 910 | 403 | 805 | CTGTGGTCGCTGCTGTGATGTGGAG | Antisense | 4109 |
| 221875_x_at | 1099 | 137 | 858 | AGCTACTCTCAGGCTGCAGTGTGAG | Antisense | 4110 |
| 221875_x_at | 67 | 773 | 878 | GTGAGACAGCTTCCTTGTGTGGGAC | Antisense | 4111 |
| 221875_x_at | 825 | 5 | 930 | ATTGCACTTGTGCCTCACGAACATA | Antisense | 4112 |
| 223168_at | 845 | 31 | 3453 | ATACTTGAGATTTTCCTCCATTTTT | Antisense | 4113 |
| 223168_at | 839 | 197 | 3568 | AAATTGTTAAACTTGCAGCTTGGTA | Antisense | 4114 |
| 223168_at | 902 | 633 | 3626 | GAGAATGCCACTTTGGCTGAACTAC | Antisense | 4115 |
| 223168_at | 500 | 173 | 3646 | ACTACAAGTGTAGGCCACCATTATA | Antisense | 4116 |
| 223168_at | 1013 | 901 | 3732 | TGGACCTTTTATAACCTTGTTCTCT | Antisense | 4117 |
| 223168_at | 1038 | 327 | 3763 | CAGACTCAAGAGAAACTACCCAGGT | Antisense | 4118 |
| 223168_at | 975 | 171 | 3777 | ACTACCCAGGTATTACACAAGCCAA | Antisense | 4119 |
| 223168_at | 751 | 205 | 3801 | AAATGGGAGCAAGGCCTTCTCTCCA | Antisense | 4120 |
| 223168_at | 406 | 361 | 3816 | CTTCTCTCCAGACTATCGTAACCTG | Antisense | 4121 |
| 223168_at | 525 | 1 | 3830 | ATCGTAACCTGGTGCCTTACCAAGT | Antisense | 4122 |
| 223168_at | 345 | 477 | 3843 | GCCTTACCAAGTTGTGCTTTTCTGT | Antisense | 4123 |
| 223593_at | 41 | 875 | 1467 | GGCAGCTGCAGACAAGTGGTTAACT | Antisense | 4124 |
| 223593_at | 336 | 595 | 1477 | GACAAGTGGTTAACTGGTTTGGCAG | Antisense | 4125 |
| 223593_at | 920 | 1159 | 1495 | TTGGCAGAATGGCATGTTCCTGCTG | Antisense | 4126 |
| 223593_at | 255 | 667 | 1593 | GATGGGGGTATTAATGCTCCCTGGA | Antisense | 4127 |
| 223593_at | 9 | 505 | 1608 | GCTCCCTGGAAATGCTTTCTACGTC | Antisense | 4128 |
| 223593_at | 1103 | 371 | 1626 | CTACGTCGATAGCTCAGCTCCTAGC | Antisense | 4129 |
| 223593_at | 458 | 981 | 1644 | TCCTAGCCCTTACTTGAGAGCATCC | Antisense | 4130 |
| 223593_at | 768 | 667 | 1696 | GATGTGGCCTTCCAGGTATTAGCAC | Antisense | 4131 |
| 223593_at | 444 | 891 | 1701 | GGCCTTCCAGGTATTAGCACAACTT | Antisense | 4132 |
| 223593_at | 635 | 171 | 1884 | ACTCTGGCCTGCATGGCAGAACAAG | Antisense | 4133 |
| 223593_at | 445 | 201 | 1934 | AAATCAAACTAATCATGCTGCTCAT | Antisense | 4134 |
| 223646_s_at | 435 | 225 | 1999 | AAAATACTGGACCCACTTCTTTCAG | Antisense | 4135 |
| 223646_s_at | 476 | 565 | 2031 | GAAGATACCTTATATGCCCTAAAGT | Antisense | 4136 |
| 223646_s_at | 1055 | 485 | 2046 | GCCCTAAAGTTAATACCAGCAGTCA | Antisense | 4137 |
| 223646_s_at | 612 | 885 | 2131 | GGCTGTGACACTTCTTTGTGGTACT | Antisense | 4138 |
| 223646_s_at | 526 | 159 | 2363 | ACCCTTTGCAGAAGATTCCCTTGTA | Antisense | 4139 |
| 223646_s_at | 989 | 1139 | 2378 | TTCCCTTGTAAATGGCCCTGTGGCA | Antisense | 4140 |
| 223646_s_at | 534 | 877 | 2399 | GGCATGCCCAGTATCTGCAATGTTC | Antisense | 4141 |
| 223646_s_at | 253 | 871 | 2437 | GGCAAACCTCTCTGTTTGCCAAGCC | Antisense | 4142 |
| 223646_s_at | 175 | 717 | 2450 | GTTTGCCAAGCCTGCAGAGTTGAAC | Antisense | 4143 |
| 223646_s_at | 979 | 1049 | 2490 | TACAATCTGACCCTGTTTTTTGGCC | Antisense | 4144 |
| 223646_s_at | 176 | 429 | 2513 | CCCTGTTTCTGGACACTGTAGCTGA | Antisense | 4145 |
| 223746_at | 1101 | 691 | 356 | GATTCCTGGTCTTTTTAACACGAAG | Antisense | 4146 |
| 223746_at | 604 | 1005 | 395 | TCTCTTTTGTAGGATCTGTATGATT | Antisense | 4147 |
| 223746_at | 583 | 15 | 541 | ATTTTAATATTTGCACCTTTGTGAA | Antisense | 4148 |
| 223746_at | 1123 | 197 | 577 | AAATTAAGCACTATCACCACCTTCA | Antisense | 4149 |
| 223746_at | 439 | 189 | 601 | ACAGCTACTTAGGAGATCCACAATC | Antisense | 4150 |
| 223746_at | 220 | 675 | 615 | GATCCACAATCCTGGGTTGGGAGCC | Antisense | 4151 |
| 223746_at | 749 | 625 | 635 | GAGCCAGTGGATTTCCTGAAACACA | Antisense | 4152 |
| 223746_at | 426 | 193 | 655 | ACACAGATTTGTTAATGCGTTTATA | Antisense | 4153 |
| 223746_at | 20 | 767 | 707 | GTGCAGTGACCATCATTGACTTTCA | Antisense | 4154 |
| 223746_at | 706 | 289 | 779 | AATTCAATCCTGTGTATTTATTCCT | Antisense | 4155 |
| 223746_at | 530 | 1105 | 795 | TTTATTCCTTCTTCTGTAATGACCA | Antisense | 4156 |
| 223819_x_at | 665 | 623 | 547 | GACCAGCTCCAGGAGCTCTGCATCC | Antisense | 4157 |
| 223819_x_at | 374 | 493 | 599 | GCGTGGTATTTGGGAGCCAGCGGCC | Antisense | 4158 |
| 223819_x_at | 24 | 1155 | 629 | TTGATTCTGTGGCCCAGCAGCAGGG | Antisense | 4159 |
| 223819_x_at | 802 | 391 | 661 | CTGCCGCATGTTGCTGACTTTCGGT | Antisense | 4160 |
| 223819_x_at | 999 | 627 | 738 | GAGCGTCCTGATGCAGCTGAAGCTT | Antisense | 4161 |
| 223819_x_at | 861 | 911 | 768 | TGGGTCAGCATACCGCTTTGAGGTC | Antisense | 4162 |
| 223819_x_at | 314 | 469 | 799 | GCCAAGTTCCAGGAGCTGCGGTACA | Antisense | 4163 |
| 223819_x_at | 1157 | 1027 | 896 | TCACTTGACCAGTCCCATTCAGATC | Antisense | 4164 |
| 223819_x_at | 1124 | 821 | 944 | GGTGCCAAAGTGCAGCTGACTCTTC | Antisense | 4165 |
| 223819_x_at | 638 | 1141 | 986 | TTCCCATGAGGCAGGCTCTTCAGTG | Antisense | 4166 |
| 223819_x_at | 986 | 629 | 1056 | GAGCTATGCCTTTTTTTCTTTTTGG | Antisense | 4167 |
| 223945_x_at | 258 | 303 | 438 | CAACATTCTCTTCTCAAACACTGAC | Antisense | 4168 |
| 223945_x_at | 417 | 917 | 527 | TGTGGGTCACAGCAGTGAGCTCCCA | Antisense | 4169 |
| 223945_x_at | 512 | 463 | 549 | CCACCCGCCTTGCAGTGAAGATGTG | Antisense | 4170 |
| 223945_x_at | 81 | 849 | 580 | GGAGAGGGAGTGTCTCCTTCCAGGT | Antisense | 4171 |
| 223945_x_at | 840 | 85 | 601 | AGGTGCTAGCTCTGGACAGCAGCTG | Antisense | 4172 |
| 223945_x_at | 587 | 859 | 636 | GGAAAGTTTCTTCATCGTTGTCCTC | Antisense | 4173 |
| 223945_x_at | 548 | 445 | 657 | CCTCCCTGCTGGTCACATGAGTTTA | Antisense | 4174 |
| 223945_x_at | 999 | 161 | 681 | ACGATTCCTTAGAAGTGTCTCCCAC | Antisense | 4175 |
| 223945_x_at | 1127 | 633 | 722 | GAGAATCTCTGAGGCGTGTCTTCCA | Antisense | 4176 |
| 223945_x_at | 591 | 969 | 764 | TGCCCTCCACAGTGTAGGCTTGAAC | Antisense | 4177 |
| 223945_x_at | 623 | 75 | 779 | AGGCTTGAACACTTGGCCTCATGCG | Antisense | 4178 |
| 224153_s_at | 1005 | 531 | 860 | GCAGAACATATGTAACCAGAAGGAC | Antisense | 4179 |
| 224153_s_at | 169 | 1153 | 970 | TTGAGTGCAAGTGGGCTGAGTCTGA | Antisense | 4180 |
| 224153_s_at | 863 | 767 | 974 | GTGCAAGTGGGCTGAGTCTGAAAAC | Antisense | 4181 |
| 224153_s_at | 896 | 263 | 1071 | AAGTGGTTATCTATTGTTAGCAGAG | Antisense | 4182 |
| 224153_s_at | 1011 | 811 | 1119 | GGGGAGATGATAAGACCCATTGTCC | Antisense | 4183 |
| 224153_s_at | 875 | 59 | 1125 | ATGATAAGACCCATTGTCCAGAAGA | Antisense | 4184 |
| 224153_s_at | 667 | 615 | 1151 | GACTGTCACAAGGTCGATTGATCAG | Antisense | 4185 |
| 224153_s_at | 450 | 605 | 1185 | GACAGGGACAGGTCACAATGGTGGA | Antisense | 4186 |
| 224153_s_at | 815 | 271 | 1201 | AATGGTGGAATGTTGTAATGTTGGT | Antisense | 4187 |
| 224153_s_at | 231 | 1103 | 1253 | TTTACTTCTTGTGTGGTTTTTCAGC | Antisense | 4188 |
| 224153_s_at | 1161 | 1131 | 1258 | TTCTTGTGTGGTTTTTCAGCTGCCC | Antisense | 4189 |
| 224908_s_at | 277 | 149 | 2301 | ACCAAGCCTGGCTTTATGTATTTAT | Antisense | 4190 |
| 224908_s_at | 343 | 187 | 2387 | ACAGAAGGTTATGCCTGGCTCCCAG | Antisense | 4191 |
| 224908_s_at | 348 | 909 | 2454 | TGGGGGAGGAGTCATGGTTTATTTG | Antisense | 4192 |
| 224908_s_at | 558 | 969 | 2549 | TGCCTTAGTTTCTTAGCCCATGAAA | Antisense | 4193 |
| 224908_s_at | 500 | 619 | 2587 | GACCCAGGGACTACCTCAAGGGCTT | Antisense | 4194 |
| 224908_s_at | 1051 | 865 | 2634 | GGAAGATGCAAGAGCCTTTAGTACC | Antisense | 4195 |
| 224908_s_at | 192 | 117 | 2644 | AGAGCCTTTAGTACCAAGGTTCTCA | Antisense | 4196 |
| 224908_s_at | 658 | 425 | 2738 | CCCGGACAGTTAAATCAGAACCTCA | Antisense | 4197 |
| 224908_s_at | 811 | 327 | 2753 | CAGAACCTCAGACAGCAATATGCCT | Antisense | 4198 |
| 224908_s_at | 319 | 21 | 2770 | ATATGCCTTGAGATGCCTTGAACCA | Antisense | 4199 |
| 224908_s_at | 464 | 643 | 2779 | GAGATGCCTTGAACCATGCTTGAGA | Antisense | 4200 |
| 225432_s_at | 553 | 887 | 3003 | GGCGCTGATGCGAATACAGCTCACA | Antisense | 4201 |
| 225432_s_at | 986 | 693 | 3081 | GATTGATTTCACAGGGAGCTCTAAT | Antisense | 4202 |
| 225432_s_at | 222 | 45 | 3104 | ATCTCTGTGATTACATGGTCCTTCA | Antisense | 4203 |
| 225432_s_at | 1095 | 449 | 3123 | CCTTCAAACTCCCAACCAAAGTGAG | Antisense | 4204 |
| 225432_s_at | 531 | 943 | 3175 | TGAGCAGCCCTTTAGCAAAATCGCC | Antisense | 4205 |
| 225432_s_at | 225 | 387 | 3273 | CTCCCTCCACCTGACAGTTTGTAAG | Antisense | 4206 |
| 225432_s_at | 688 | 43 | 3309 | ATCTAACCTGATGCTCTTGGAGAGA | Antisense | 4207 |
| 225432_s_at | 991 | 643 | 3332 | GAGATAACCTGTCTGTCATAACTTA | Antisense | 4208 |
| 225432_s_at | 654 | 513 | 3379 | GCTATTAAAGATTCATGCAGTCCCA | Antisense | 4209 |
| 225432_s_at | 409 | 959 | 3394 | TGCAGTCCCAAAAGGCACTGTCCTG | Antisense | 4210 |
| 225432_s_at | 847 | 349 | 3446 | CATAAAAGGAATCCAACCCTGTGCC | Antisense | 4211 |
| 225502_at | 1153 | 571 | 5134 | GAAGCCTTACTACAATTCCAAAAAT | Antisense | 4212 |
| 225502_at | 895 | 39 | 5157 | ATCATCATGGTTGGAAATTTGGGAG | Antisense | 4213 |
| 225502_at | 522 | 1085 | 5187 | TATTTGTGAACTTGTTACCCTTTTG | Antisense | 4214 |
| 225502_at | 162 | 741 | 5212 | GTAATGGTGGACTAATTGCTGTATA | Antisense | 4215 |
| 225502_at | 885 | 1051 | 5319 | TACACGGTACTTGGAGCAGTCAGCC | Antisense | 4216 |
| 225502_at | 659 | 1029 | 5349 | TCACAGATACTGCTTTCACTTAAAT | Antisense | 4217 |
| 225502_at | 87 | 291 | 5380 | AATTCTCCGATAATGCTTTGCTTTT | Antisense | 4218 |
| 225502_at | 1017 | 743 | 5413 | GTCACTCTTGTGTACTATCTATTTT | Antisense | 4219 |
| 225502_at | 324 | 1159 | 5420 | TTGTGTACTATCTATTTTTCTCCTC | Antisense | 4220 |
| 225502_at | 283 | 251 | 5469 | AAGCAATAATATCTCTGTTTTCATT | Antisense | 4221 |
| 225502_at | 218 | 45 | 5479 | ATCTCTGTTTTCATTTCAGAACATT | Antisense | 4222 |
| 225736_at | 96 | 445 | 675 | CCTCTGGATATTGATGCCTCGGGTG | Antisense | 4223 |
| 225736_at | 1112 | 899 | 719 | TGGACACCGAATCCAGAGTGCCACT | Antisense | 4224 |
| 225736_at | 478 | 315 | 740 | CACTGTGCTCCTCAACGAGGACGTC | Antisense | 4225 |
| 225736_at | 837 | 671 | 768 | GATGAGAAGACTGCTGAGGCTGCGA | Antisense | 4226 |
| 225736_at | 83 | 939 | 782 | TGAGGCTGCGATGCAGCGCCTCAAA | Antisense | 4227 |
| 225736_at | 743 | 211 | 804 | AAAGCGGCCAACATTCCAGAGCATA | Antisense | 4228 |
| 225736_at | 596 | 631 | 822 | GAGCATAACACCATTGGCTTCATGT | Antisense | 4229 |
| 225736_at | 659 | 879 | 837 | GGCTTCATGTTTGCATGCGTTGGCA | Antisense | 4230 |
| 225736_at | 157 | 695 | 855 | GTTGGCAGGGGCTTTCAGTATTACA | Antisense | 4231 |
| 225736_at | 584 | 987 | 932 | TCCCTTATTCGGCTTCTTTGGAAAT | Antisense | 4232 |
| 225736_at | 225 | 71 | 1056 | ATGGCACTCATACATCTGGGGTCAT | Antisense | 4233 |
| 225853_at | 248 | 181 | 3372 | ACATATGTCGGCTACCGTGTCTGGC | Antisense | 4234 |
| 225853_at | 178 | 923 | 3389 | TGTCTGGCCGTTCACATCTTTGGCC | Antisense | 4235 |
| 225853_at | 214 | 181 | 3402 | ACATCTTTGGCCACTATTTGCTTGT | Antisense | 4236 |
| 225853_at | 336 | 1039 | 3499 | TAAGATGCACTGTAACATCTCTGAA | Antisense | 4237 |
| 225853_at | 1044 | 711 | 3554 | GTTTTGTTAGCAATACACTGTCTTT | Antisense | 4238 |
| 225853_at | 414 | 1051 | 3567 | TACACTGTCTTTTAGTGGTGCCTAA | Antisense | 4239 |
| 225853_at | 838 | 569 | 3629 | GAAGCACAGTATGCAGGTAGGCCTA | Antisense | 4240 |
| 225853_at | 583 | 527 | 3641 | GCAGGTAGGCCTAATGGGGGAAGAT | Antisense | 4241 |
| 225853_at | 519 | 247 | 3709 | AAGCTGTTCTGTGGATGACCTACCC | Antisense | 4242 |
| 225853_at | 933 | 1123 | 3740 | TTAAACACGATTCTCTCACTTCCAA | Antisense | 4243 |
| 225853_at | 1134 | 465 | 3761 | CCAACTCCAAACTTGCTCAACTAAT | Antisense | 4244 |
| 225882_at | 1108 | 535 | 2178 | GCACCTTACATATTTGATGCTCAGG | Antisense | 4245 |
| 225882_at | 944 | 461 | 2283 | CCACCCTTACATCTCCATAGTTGGT | Antisense | 4246 |
| 225882_at | 1067 | 695 | 2302 | GTTGGTACAGTTAGCTTGTAGCAGC | Antisense | 4247 |
| 225882_at | 561 | 51 | 2335 | ATGCCTGGAGATCACTGTCTGTTGG | Antisense | 4248 |
| 225882_at | 429 | 401 | 2349 | CTGTCTGTTGGTCTGATCTCAGTAT | Antisense | 4249 |
| 225882_at | 844 | 333 | 2368 | CAGTATCATACTGAGACACCTCCCC | Antisense | 4250 |
| 225882_at | 909 | 441 | 2391 | CCTGAGCCTTACCTACTTAAATTGG | Antisense | 4251 |
| 225882_at | 899 | 661 | 2453 | GAGGCAAGTTGCAAGGGAGCCAGAG | Antisense | 4252 |
| 225882_at | 841 | 285 | 2534 | AATTTCTTTGGCACATTGACTTACT | Antisense | 4253 |
| 225882_at | 169 | 873 | 2543 | GGCACATTGACTTACTGATATCTTT | Antisense | 4254 |
| 225882_at | 408 | 1001 | 2614 | TATTCCCCGGCGCAATAAAAATGCC | Antisense | 4255 |
| 226043_at | 966 | 89 | 2223 | AGTGGGGGGGTGGTCAGTGGAAGCT | Antisense | 4256 |
| 226043_at | 952 | 141 | 2244 | AGCTGGGGAGGCTTCACTCAGCTCA | Antisense | 4257 |
| 226043_at | 871 | 1027 | 2327 | TCACTGCTCTCTCCATGGGGAGGGG | Antisense | 4258 |
| 226043_at | 609 | 337 | 2357 | CAGGGCACCAAGTTCGGGATCCTAG | Antisense | 4259 |
| 226043_at | 79 | 435 | 2542 | CCCCACTCTCAGGCTAGGATGGAGA | Antisense | 4260 |
| 226043_at | 710 | 431 | 2667 | CCCCCCCGCATACTTGAATGTATGT | Antisense | 4261 |
| 226043_at | 11 | 727 | 2686 | GTATGTGCGTATTTATTGCTCACGT | Antisense | 4262 |
| 226043_at | 432 | 17 | 2696 | ATTTATTGCTCACGTCTGTGCCATG | Antisense | 4263 |
| 226043_at | 664 | 1027 | 2705 | TCACGTCTGTGCCATGTTGTCAATG | Antisense | 4264 |
| 226043_at | 534 | 931 | 2719 | TGTTGTCAATGGGTCCTTTCCAACC | Antisense | 4265 |
| 226043_at | 589 | 365 | 2734 | CTTTCCAACCCAAGAGGTACATTTG | Antisense | 4266 |
| 226778_at | 173 | 589 | 1803 | GAAACTCTGGAAATCACGTGTGTGG | Antisense | 4267 |
| 226778_at | 72 | 917 | 1823 | TGTGGGGAGATGGGGACGCTTCCCA | Antisense | 4268 |
| 226778_at | 159 | 841 | 1836 | GGACGCTTCCCATGTTGTGGGGAGC | Antisense | 4269 |
| 226778_at | 1070 | 423 | 1844 | CCCATGTTGTGGGGAGCTCTGTGGC | Antisense | 4270 |
| 226778_at | 1052 | 441 | 1912 | CCTGCAACTCACGTCAATCATAGAA | Antisense | 4271 |
| 226778_at | 262 | 3 | 1936 | ATTGTGACGCACAGTTGGCAAAATA | Antisense | 4272 |
| 226778_at | 13 | 1071 | 1959 | TAGTTCTTTATGCTATTTCTCAAAA | Antisense | 4273 |
| 226778_at | 323 | 657 | 1988 | GAGGACAAACCCAGATTGGGATTGG | Antisense | 4274 |
| 226778_at | 397 | 1159 | 2003 | TTGGGATTGGAATATGCACTGTAAA | Antisense | 4275 |
| 226778_at | 430 | 955 | 2017 | TGCACTGTAAATCAAATTTTTCTTA | Antisense | 4276 |
| 226778_at | 1068 | 915 | 2231 | TGTGTAAACACACAGCTTCCTTGTG | Antisense | 4277 |
| 227035_x_at | 257 | 303 | 809 | CAACATTCTCTTCTCAAACACTGAC | Antisense | 4278 |
| 227035_x_at | 418 | 917 | 898 | TGTGGGTCACAGCAGTGAGCTCCCA | Antisense | 4279 |
| 227035_x_at | 511 | 463 | 920 | CCACCCGCCTTGCAGTGAAGATGTG | Antisense | 4280 |
| 227035_x_at | 83 | 849 | 951 | GGAGAGGGAGTGTCTCCTTCCAGGT | Antisense | 4281 |
| 227035_x_at | 839 | 85 | 972 | AGGTGCTAGCTCTGGACAGCAGCTG | Antisense | 4282 |
| 227035_x_at | 585 | 859 | 1007 | GGAAAGTTTCTTCATCGTTGTCCTC | Antisense | 4283 |
| 227035_x_at | 547 | 445 | 1028 | CCTCCCTGCTGGTCACATGAGTTTA | Antisense | 4284 |
| 227035_x_at | 1000 | 161 | 1052 | ACGATTCCTTAGAAGTGTCTCCCAC | Antisense | 4285 |
| 227035_x_at | 1126 | 633 | 1093 | GAGAATCTCTGAGGCGTGTCTTCCA | Antisense | 4286 |
| 227035_x_at | 881 | 919 | 1133 | TGTGCCCTCCACAGTGTAGACTTGA | Antisense | 4287 |
| 227035_x_at | 87 | 229 | 1157 | AACACTTGGCCTCATGTGATCCTTC | Antisense | 4288 |
| 228376_at | 851 | 823 | 516 | GGTCAGGGGATCAGACACTCCAGTT | Antisense | 4289 |
| 228376_at | 993 | 267 | 560 | AATGGCAAAAGGCTCCATACCCTTC | Antisense | 4290 |
| 228376_at | 1038 | 797 | 620 | GGGAATGATGATAACACCCACTTCA | Antisense | 4291 |
| 228376_at | 1110 | 227 | 632 | AACACCCACTTCATAGAATGGTCAT | Antisense | 4292 |
| 228376_at | 1111 | 741 | 680 | GTCAAGCACTTAGCCTCTGGTGCAC | Antisense | 4293 |
| 228376_at | 872 | 1047 | 714 | TAAATAAGTATACCTATTCCTCCTT | Antisense | 4294 |
| 228376_at | 977 | 21 | 756 | ATATTACCAAATGTCCAGCTTATAC | Antisense | 4295 |
| 228376_at | 868 | 511 | 797 | GCTAGTGGGCTATGTTAGAGCTACT | Antisense | 4296 |
| 228376_at | 693 | 121 | 913 | AGACAGCTGTGTGGGTGTTTTTTGG | Antisense | 4297 |
| 228376_at | 654 | 997 | 947 | TCTGGTTTACAATCTCGTCATTCAA | Antisense | 4298 |
| 228376_at | 229 | 75 | 1034 | AGGCTCATCATTTCTCAGTCTGAAT | Antisense | 4299 |
| 228604_at | 845 | 593 | 622 | GAAATGGTGGTTTCCTTCTGATAAG | Antisense | 4300 |
| 228604_at | 731 | 853 | 656 | GGACCATGTCGCTTATATATCACCC | Antisense | 4301 |
| 228604_at | 1139 | 415 | 665 | CGCTTATATATCACCCTACTAGGGG | Antisense | 4302 |
| 228604_at | 898 | 685 | 689 | GATATAATTTTCCCTTCTAAGCAAG | Antisense | 4303 |
| 228604_at | 313 | 271 | 717 | AATGGCAGTGCTAAATAGTTTTGTA | Antisense | 4304 |
| 228604_at | 299 | 287 | 785 | AATATTTTTCAGATGCATTTGCATT | Antisense | 4305 |
| 228604_at | 637 | 855 | 868 | GGACTGAAATTCTGTTACATGATGT | Antisense | 4306 |
| 228604_at | 585 | 23 | 954 | ATATAAAGCAAATTCACACGTTTTC | Antisense | 4307 |
| 228604_at | 865 | 589 | 1011 | GAAACCTCAGTCTTGAAATTTTGAT | Antisense | 4308 |
| 228604_at | 786 | 219 | 1040 | AAAAATCATGACACTGTTTTACCAT | Antisense | 4309 |
| 228604_at | 882 | 645 | 1071 | GAGTAGCTAACTTTTGGTAACACCT | Antisense | 4310 |
| 228786_at | 359 | 309 | 317 | CACACGCAGATCCTGCACTGTACTG | Antisense | 4311 |
| 228786_at | 819 | 49 | 326 | ATCCTGCACTGTACTGTCCAGTCAA | Antisense | 4312 |
| 228786_at | 1007 | 165 | 338 | ACTGTCCAGTCAACTGACTTCAGCA | Antisense | 4313 |
| 228786_at | 645 | 665 | 365 | GATGTCTGGGTTCAGCAGCGAGTTG | Antisense | 4314 |
| 228786_at | 1099 | 645 | 384 | GAGTTGCTGTGTGAGGCCACCAGAA | Antisense | 4315 |
| 228786_at | 198 | 803 | 494 | GGGAGACATTCAGAATCGAGGCGAA | Antisense | 4316 |
| 228786_at | 807 | 447 | 524 | CCTCAGTGCTCCTCTTGGAGGCAGA | Antisense | 4317 |
| 228786_at | 699 | 767 | 583 | GTGAATGTGGAAGACCAGCAGATTG | Antisense | 4318 |
| 228786_at | 1088 | 785 | 665 | GTGGTGAAGTGGCCCTGCACAAAAC | Antisense | 4319 |
| 228786_at | 679 | 263 | 671 | AAGTGGCCCTGCACAAAACGTTGCT | Antisense | 4320 |
| 228786_at | 332 | 1071 | 817 | TAGTAAAGCTAAGTTTTATCTCTTC | Antisense | 4321 |
| 228812_at | 843 | 637 | 765 | GAGACTTCATTGGTGATACACTCAA | Antisense | 4322 |
| 228812_at | 406 | 775 | 777 | GTGATACACTCAATTTTTACTGGGT | Antisense | 4323 |
| 228812_at | 488 | 509 | 807 | GCTAATAATGTTGGTCACTGTCTCA | Antisense | 4324 |
| 228812_at | 742 | 301 | 869 | CACAAAGCTTTTGGGTAACCAGCGT | Antisense | 4325 |
| 228812_at | 873 | 805 | 881 | GGGTAACCAGCGTTCTTAAATGTAT | Antisense | 4326 |
| 228812_at | 552 | 1075 | 903 | TATGGTTTTTGACCAGGTGAACCCT | Antisense | 4327 |
| 228812_at | 775 | 623 | 913 | GACCAGGTGAACCCTTTAGAAGTGA | Antisense | 4328 |
| 228812_at | 1155 | 199 | 963 | AAATACCTTTGGCTGTGATGAATGT | Antisense | 4329 |
| 228812_at | 1101 | 953 | 981 | TGAATGTAGATCCCAGCAGAATACC | Antisense | 4330 |
| 228812_at | 137 | 1153 | 1021 | TTGACTGAGTATTTGTAGATGCTTA | Antisense | 4331 |
| 228812_at | 198 | 259 | 1091 | AAGTTCTCAGGTACTGTTCAATTAT | Antisense | 4332 |
| 229598_at | 248 | 15 | 566 | ATTTTTCTTAGCTAAATCTGGCAAC | Antisense | 4333 |
| 229598_at | 573 | 133 | 636 | AGCAGAATCTGCCATATGAGTAATA | Antisense | 4334 |
| 229598_at | 259 | 649 | 653 | GAGTAATAGAAGTGAGCAGGCCCAG | Antisense | 4335 |
| 229598_at | 304 | 335 | 675 | CAGGACTCCCTAAGTCAAGAAACCA | Antisense | 4336 |
| 229598_at | 19 | 121 | 692 | AGAAACCAAGAGGCGTCATTACGGA | Antisense | 4337 |
| 229598_at | 549 | 209 | 717 | AAAGAGTAACTCACCCTGTGTGCTC | Antisense | 4338 |
| 229598_at | 90 | 143 | 759 | AGCGATGCCCCCATGTTATGAATGG | Antisense | 4339 |
| 229598_at | 747 | 859 | 784 | GGAAAAGTTCACTGAAGGGTTCATA | Antisense | 4340 |
| 229598_at | 796 | 1161 | 846 | TTGGATACCTTCAAGGGATCAGAAA | Antisense | 4341 |
| 229598_at | 394 | 1017 | 899 | TCATCATTACTCTCTACATTATTAT | Antisense | 4342 |
| 229598_at | 448 | 1109 | 983 | TTTTAACTTGTCTGCATGTTTGAAA | Antisense | 4343 |
| 231229_at | 843 | 533 | 31 | GCACGTCCAAGGTGATCCTGAGGGC | Antisense | 4344 |
| 231229_at | 969 | 317 | 96 | CACCCTGAAGAAGGCTGTTTCCACC | Antisense | 4345 |
| 231229_at | 999 | 605 | 130 | GACATGGCCCGAAATGCCTATCACT | Antisense | 4346 |
| 231229_at | 632 | 477 | 145 | GCCTATCACTTCAAGCGTGTGCTCA | Antisense | 4347 |
| 231229_at | 105 | 389 | 247 | CTCCAAGTCCAAGCTCAAGGTCAAG | Antisense | 4348 |
| 231229_at | 658 | 603 | 315 | GACAGCACAGGTCACTACTGGGCTC | Antisense | 4349 |
| 231229_at | 453 | 1057 | 330 | TACTGGGCTCCAAACAGGGGCACAA | Antisense | 4350 |
| 231229_at | 908 | 293 | 352 | CAAGCGGCTTATCAAGGGGGTTCGA | Antisense | 4351 |
| 231229_at | 961 | 803 | 369 | GGGTTCGAAGGGTGGCCAAGTGCCA | Antisense | 4352 |
| 231229_at | 847 | 59 | 403 | ATGAGGCAGGCCAGGCAAGCAGTCA | Antisense | 4353 |
| 231229_at | 1130 | 419 | 441 | CGCCATTGGCTCAGTGCAGTGGGAA | Antisense | 4354 |
| 232019_at | 931 | 445 | 1508 | CCTCGGCTCCTCATCTGAAGAGAAG | Antisense | 4355 |
| 232019_at | 1103 | 1041 | 1540 | TAACTGCTAAGTTGCTCTGGCTCTA | Antisense | 4356 |
| 232019_at | 521 | 1149 | 1551 | TTGCTCTGGCTCTAATGTTTCATGA | Antisense | 4357 |
| 232019_at | 725 | 973 | 1645 | TGCCACATCTGGCCATACCAAATCC | Antisense | 4358 |
| 232019_at | 813 | 149 | 1661 | ACCAAATCCATCGCATGCTTCAGGA | Antisense | 4359 |
| 232019_at | 447 | 593 | 1684 | GAAAGGGAGGGGATGCTGTGACAGC | Antisense | 4360 |
| 232019_at | 51 | 635 | 1738 | GAGAAAGGGCAAGTCCCTCGTGGTC | Antisense | 4361 |
| 232019_at | 114 | 421 | 1777 | CCCAAGCACCTCGATAACCAGTGGC | Antisense | 4362 |
| 232019_at | 717 | 887 | 1841 | GGCGCCCAGTGACAGACAAGACAGC | Antisense | 4363 |
| 232019_at | 650 | 141 | 1874 | AGCTGGGGCAGCCCTGGCTAAAGGA | Antisense | 4364 |
| 232019_at | 231 | 383 | 1928 | CTCTGCCTCTGAATGCATTCCAAGA | Antisense | 4365 |
| 232311_at | 229 | 181 | 2003 | ACATACCTTGGGTTGATCCACTTAG | Antisense | 4366 |
| 232311_at | 757 | 675 | 2017 | GATCCACTTAGGAACCTCAGATAAT | Antisense | 4367 |
| 232311_at | 930 | 231 | 2042 | AACATCTGCCACGTATAGAGCAATT | Antisense | 4368 |
| 232311_at | 444 | 1071 | 2069 | TATGTCCCAGGCACTCTACTAGACA | Antisense | 4369 |
| 232311_at | 999 | 371 | 2084 | CTACTAGACACTTCATACAGTTTAG | Antisense | 4370 |
| 232311_at | 33 | 811 | 2119 | GGGTGTAGATCAAGGCAGGAGCAGG | Antisense | 4371 |
| 232311_at | 387 | 667 | 2220 | GATGGGGGCTATTATGAACTGAGAA | Antisense | 4372 |
| 232311_at | 683 | 1159 | 2264 | TTGGGGCCAAATCATGTAGACTCTT | Antisense | 4373 |
| 232311_at | 543 | 1067 | 2280 | TAGACTCTTGAGTGATGTGTTAAGG | Antisense | 4374 |
| 232311_at | 137 | 869 | 2303 | GGAATGCTATGAGTGCTGAGAGGGC | Antisense | 4375 |
| 232311_at | 1085 | 521 | 2326 | GCATCAGAAGTCCTTGAGAGCCTCC | Antisense | 4376 |
| 235391_at | 477 | 17 | 281 | ATTTCACGGCAAAGCTTTAGAGGTC | Antisense | 4377 |
| 235391_at | 482 | 1101 | 296 | TTTAGAGGTCTACACTGCTGCCTAC | Antisense | 4378 |
| 235391_at | 458 | 1051 | 306 | TACACTGCTGCCTACCAGAATATAC | Antisense | 4379 |
| 235391_at | 862 | 289 | 369 | AATTCTCTGTATGCACCAGATTATT | Antisense | 4380 |
| 235391_at | 758 | 541 | 417 | GCAAATTCAAAGTCACCTCTTCAGA | Antisense | 4381 |
| 235391_at | 16 | 155 | 431 | ACCTCTTCAGAGATCACTGTCAGCT | Antisense | 4382 |
| 235391_at | 439 | 167 | 446 | ACTGTCAGCTAAGTGTGTATCTGGA | Antisense | 4383 |
| 235391_at | 569 | 185 | 476 | ACAGGTATCCACTTGTCGACTAAGA | Antisense | 4384 |
| 235391_at | 29 | 1123 | 565 | TTAAGTAAACTACACATTTCCATTT | Antisense | 4385 |
| 235391_at | 985 | 579 | 630 | GAACTTTATACTCACTTTGCTATGT | Antisense | 4386 |
| 235391_at | 369 | 245 | 656 | AAGCCTCAAAGTGAAGTCCAACTGG | Antisense | 4387 |
| 235421_at | 755 | 29 | 109 | ATACCTTCACAATCTGAACTTTCTC | Antisense | 4388 |
| 235421_at | 770 | 579 | 124 | GAACTTTCTCTAGATGGGCACAGAT | Antisense | 4389 |
| 235421_at | 627 | 95 | 201 | AGTGCTGAGGCTTTTTGCAGGCGCA | Antisense | 4390 |
| 235421_at | 682 | 957 | 216 | TGCAGGCGCAGTTAGCAGGCGAACT | Antisense | 4391 |
| 235421_at | 1114 | 883 | 233 | GGCGAACTGGTCAGCAGGGTTTCTG | Antisense | 4392 |
| 235421_at | 1015 | 409 | 263 | CGTAGATTTGCGTGACAGGGGCCCT | Antisense | 4393 |
| 235421_at | 1142 | 337 | 278 | CAGGGGCCCTGTGTTGCTAAGGAAT | Antisense | 4394 |
| 235421_at | 900 | 223 | 319 | AAAAGTCTCAGCGTCAGACACTCCT | Antisense | 4395 |
| 235421_at | 849 | 449 | 376 | CCTTTCCAGGCAACTCACAGTTTAG | Antisense | 4396 |
| 235421_at | 331 | 697 | 408 | GTTGTGTGTGTTCAAGCTCTTGGGT | Antisense | 4397 |
| 235421_at | 1103 | 231 | 477 | AACATCTTGTCTTACAGCTCTTTTA | Antisense | 4398 |
| 235473_at | 89 | 859 | 256 | GGAAATCAAGATCTGCGTGCTGGGT | Antisense | 4399 |
| 235473_at | 467 | 493 | 270 | GCGTGCTGGGTATGTTCATTGCTAC | Antisense | 4400 |
| 235473_at | 253 | 961 | 306 | TGCTTCTAGGCCTTTTCAACTGACA | Antisense | 4401 |
| 235473_at | 335 | 221 | 347 | AAAACTCATTGTAATCGTGTAACTT | Antisense | 4402 |
| 235473_at | 854 | 175 | 391 | ACTTTGCTAGTACCCCAGAAGCTCC | Antisense | 4403 |
| 235473_at | 385 | 1109 | 433 | TTTTAACGCTTAATCACCTTCATCC | Antisense | 4404 |
| 235473_at | 1120 | 319 | 447 | CACCTTCATCCTCAGTTTTATGGTA | Antisense | 4405 |
| 235473_at | 773 | 1115 | 464 | TTATGGTAATCACTTGTCTTTGCAG | Antisense | 4406 |
| 235473_at | 199 | 751 | 479 | GTCTTTGCAGTTTTAGTCTGTAACC | Antisense | 4407 |
| 235473_at | 884 | 1071 | 492 | TAGTCTGTAACCATGCATCCCTAAA | Antisense | 4408 |
| 235473_at | 676 | 177 | 583 | ACTTCTTTTATGTCTGTGATGTTCA | Antisense | 4409 |
| 235688_s_at | 55 | 939 | 27 | TGAGGCAGTGCATGTTCTTGGCCCA | Antisense | 4410 |
| 235688_s_at | 722 | 895 | 45 | TGGCCCAGAGTAAGTGCTTAGTGAA | Antisense | 4411 |
| 235688_s_at | 52 | 91 | 64 | AGTGAATGCTTTCTAACTCCGAACC | Antisense | 4412 |
| 235688_s_at | 1104 | 809 | 108 | GGGTGTTGAGCAAAAGGGGCCTTCA | Antisense | 4413 |
| 235688_s_at | 1084 | 813 | 123 | GGGGCCTTCAAGATGTTCAAGGCAC | Antisense | 4414 |
| 235688_s_at | 178 | 891 | 186 | GGCCTCAGTGGGTGCATGTGATTAT | Antisense | 4415 |
| 235688_s_at | 45 | 935 | 204 | TGATTATCCACGTTTCACCTATGAA | Antisense | 4416 |
| 235688_s_at | 773 | 601 | 276 | GACAGGGCCTCGATTCTGTTTTAAA | Antisense | 4417 |
| 235688_s_at | 678 | 739 | 290 | TCTGTTTTAAACTCCAGTAGTCCCT | Antisense | 4418 |
| 235688_s_at | 1024 | 443 | 396 | CCTCCATTACCGTCACTGGTGAAAT | Antisense | 4419 |
| 235688_s_at | 1100 | 165 | 410 | ACTGGTGAAATGCGGCTCACCTCCC | Antisense | 4420 |
| 236203_at | 84 | 63 | 21 | ATGTCAGGTTTGTACCTACCACATT | Antisense | 4421 |
| 236203_at | 116 | 873 | 107 | GGCACATAGTAAGTAGTCGATAGGT | Antisense | 4422 |
| 236203_at | 194 | 3 | 148 | ATTGTTATTTTCTGGAGTCCAACTA | Antisense | 4423 |
| 236203_at | 963 | 647 | 162 | GAGTCCAACTAACAAATCCCACAGT | Antisense | 4424 |
| 236203_at | 211 | 203 | 175 | AAATCCCACAGTGAATGACATCACA | Antisense | 4425 |
| 236203_at | 87 | 803 | 200 | GGGATGCAACCAACAAGATCCAGAA | Antisense | 4426 |
| 236203_at | 252 | 177 | 232 | ACTTCTACTAGATAAACAACTCCAT | Antisense | 4427 |
| 236203_at | 12 | 683 | 242 | GATAAACAACTCCATTTCTTCAGCA | Antisense | 4428 |
| 236203_at | 472 | 573 | 326 | GAACCAAATTTGTATGAGGCAATCA | Antisense | 4429 |
| 236203_at | 990 | 221 | 353 | AAAACTGACACCGACTGTATTAAGG | Antisense | 4430 |
| 236203_at | 919 | 59 | 401 | ATGAGATTGCTGTTATGTTTTCTAA | Antisense | 4431 |
| 236627_at | 256 | 235 | 92 | AACTAGATTTTTGACCCTCACTCAT | Antisense | 4432 |
| 236627_at | 901 | 367 | 121 | CTTTCCTTGCTATAGACATGCTTTG | Antisense | 4433 |
| 236627_at | 995 | 119 | 134 | AGACATGCTTTGCATACCCAGCTTC | Antisense | 4434 |
| 236627_at | 1111 | 1133 | 156 | TTCTAGCCACATCTTCCCTAAGAAA | Antisense | 4435 |
| 236627_at | 190 | 839 | 230 | GGATCTGCATGTTGTCAGCTACCTA | Antisense | 4436 |
| 236627_at | 326 | 697 | 240 | GTTGTCAGCTACCTAAAGCAGCAAA | Antisense | 4437 |
| 236627_at | 400 | 1045 | 281 | TAAAATCAGGGAACCTTGCAGTTTA | Antisense | 4438 |
| 236627_at | 701 | 243 | 292 | AACCTTGCAGTTTAAGGCACACCTT | Antisense | 4439 |
| 236627_at | 1156 | 1101 | 329 | TTTAATCCTTCAATGCATCCAGCCA | Antisense | 4440 |
| 236627_at | 1062 | 979 | 334 | TCCTTCAATGCATCCAGCCAAAACA | Antisense | 4441 |
| 236627_at | 149 | 1063 | 375 | TACCATCCATCAATGCAAGGTTTTT | Antisense | 4442 |
| 236681_at | 179 | 589 | 250 | GAAACTCATGTGTCCTCATGGATCG | Antisense | 4443 |
| 236681_at | 133 | 343 | 266 | CATGGATCGTGGATGCCTTCATTTC | Antisense | 4444 |
| 236681_at | 1146 | 1021 | 326 | TCAGTAGAGTGACCCGCGGATGGCA | Antisense | 4445 |
| 236681_at | 447 | 69 | 345 | ATGGCATAAATGCACCTCCTTTTCT | Antisense | 4446 |
| 236681_at | 729 | 1015 | 367 | TCTTGGCCTTGGATCTATGGGTCTG | Antisense | 4447 |
| 236681_at | 409 | 835 | 392 | GGATTGTGGTCATCTCCTCAATCCT | Antisense | 4448 |
| 236681_at | 882 | 501 | 426 | GCTGAATCAATGTGGCCGTGGGTGG | Antisense | 4449 |
| 236681_at | 121 | 889 | 439 | GGCCGTGGGTGGGAACTTACATACA | Antisense | 4450 |
| 236681_at | 896 | 613 | 482 | GACTGTCTAAACAAGGGGGCCTCGC | Antisense | 4451 |
| 236681_at | 1013 | 789 | 498 | GGGCCTCGCATGGAGCTGTAAAGCA | Antisense | 4452 |
| 236681_at | 522 | 805 | 669 | GGGATACATGGGCAGTGCTTCAGAC | Antisense | 4453 |
| 237439_at | 392 | 1087 | 20 | TATTTTTAATGCCTTTTCCTGGGAT | Antisense | 4454 |
| 237439_at | 5 | 1109 | 23 | TTTTAATGCCTTTTCCTGGGATAAG | Antisense | 4455 |
| 237439_at | 800 | 1125 | 25 | TTAATGCCTTTTCCTGGGATAAGCA | Antisense | 4456 |
| 237439_at | 191 | 53 | 28 | ATGCCTTTTCCTGGGATAAGCATTA | Antisense | 4457 |
| 237439_at | 434 | 477 | 30 | GCCTTTTCCTGGGATAAGCATTAAA | Antisense | 4458 |
| 237439_at | 279 | 685 | 87 | GATAGTGATGGTAAGGCAAGATTCT | Antisense | 4459 |
| 237439_at | 478 | 665 | 93 | GATGGTAAGGCAAGATTCTAGCAAA | Antisense | 4460 |
| 237439_at | 1063 | 869 | 101 | GGCAAGATTCTAGCAAAGAGAGATG | Antisense | 4461 |
| 237439_at | 636 | 641 | 118 | GAGAGATGGGAGATAAATGGCTGAG | Antisense | 4462 |
| 237439_at | 44 | 803 | 125 | GGGAGATAAATGGCTGAGAGTTCAG | Antisense | 4463 |
| 237439_at | 29 | 271 | 133 | AATGGCTGAGAGTTCAGGTGAATAT | Antisense | 4464 |
| 238925_at | 156 | 875 | 317 | GGCAGCAGTGGCATGGACAAGCAAC | Antisense | 4465 |
| 238925_at | 1030 | 129 | 336 | AGCAACTGCTAATTCGAGACTTACT | Antisense | 4466 |
| 238925_at | 768 | 415 | 350 | CGAGACTTACTATTGGCTTCACAGC | Antisense | 4467 |
| 238925_at | 577 | 309 | 369 | CACAGCACACCCTACAGTGAGCAGG | Antisense | 4468 |
| 238925_at | 594 | 57 | 397 | ATGAGACCGTGGAACAGCTACCTGC | Antisense | 4469 |
| 238925_at | 4 | 863 | 407 | GGAACAGCTACCTGCTTTTGGTTAA | Antisense | 4470 |
| 238925_at | 122 | 57 | 493 | ATGAACTCAGCTTGTTTTTCCTAAT | Antisense | 4471 |
| 238925_at | 315 | 289 | 578 | AATTAGCACAGTCCCTGGATCTCAA | Antisense | 4472 |
| 238925_at | 731 | 1153 | 674 | TTGATTGATCAAGAGTGCCACGGAA | Antisense | 4473 |
| 238925_at | 191 | 811 | 717 | GGGTGGAGGAGGGTGTCACTTCAGA | Antisense | 4474 |
| 238925_at | 1109 | 923 | 730 | TGTCACTTCAGATGGGGCAGGCACA | Antisense | 4475 |
| 239486_at | 540 | 1043 | 156 | TAAAGTTGGGCAGTAATCTTAATTA | Antisense | 4476 |
| 239486_at | 1024 | 929 | 224 | TGTTTTACAACTAGAACCTGCCCTA | Antisense | 4477 |
| 239486_at | 21 | 1067 | 235 | TAGAACCTGCCCTAAATGTTGAATA | Antisense | 4478 |
| 239486_at | 826 | 273 | 249 | AATGTTGAATATCTTCCTAGCAAGA | Antisense | 4479 |
| 239486_at | 754 | 359 | 261 | CTTCCTAGCAAGAAACAGTCTGTCA | Antisense | 4480 |
| 239486_at | 374 | 335 | 276 | CAGTCTGTCATTTTACTTACACGAT | Antisense | 4481 |
| 239486_at | 1134 | 171 | 290 | ACTTACACGATGTCTAACCAAACCA | Antisense | 4482 |
| 239486_at | 871 | 745 | 301 | GTCTAACCAAACCATAACTTTACAT | Antisense | 4483 |
| 239486_at | 37 | 1053 | 321 | TACATAAACTAGTCGTTTCGGTCAA | Antisense | 4484 |
| 239486_at | 933 | 619 | 467 | GACCCCTGAGTACCATTAATATTCC | Antisense | 4485 |
| 239486_at | 874 | 631 | 543 | GAGCAGCCATTATGAAATCTCACAA | Antisense | 4486 |
| 241670_x_at | 852 | 1147 | 93 | TTGCCTGCGGAGCACGGGTGGGCAT | Antisense | 4487 |
| 241670_x_at | 862 | 969 | 94 | TGCCTGCGGAGCACGGGTGGGCATT | Antisense | 4488 |
| 241670_x_at | 1033 | 391 | 97 | CTGCGGAGCACGGGTGGGCATTGGA | Antisense | 4489 |
| 241670_x_at | 173 | 851 | 101 | GGAGCACGGGTGGGCATTGGATGTA | Antisense | 4490 |
| 241670_x_at | 1085 | 535 | 104 | GCACGGGTGGGCATTGGATGTAAGG | Antisense | 4491 |
| 241670_x_at | 856 | 813 | 109 | GGTGGGCATTGGATGTAAGGTATTC | Antisense | 4492 |
| 241670_x_at | 659 | 903 | 118 | TGGATGTAAGGTATTCCTAACTTCC | Antisense | 4493 |
| 241670_x_at | 418 | 849 | 333 | GGAGACGGCAGGAGCAGCTCAGGTA | Antisense | 4494 |
| 241670_x_at | 212 | 851 | 343 | GGAGCAGCTCAGGTAACCAGGGGAA | Antisense | 4495 |
| 241670_x_at | 953 | 533 | 346 | GCAGCTCAGGTAACCAGGGGAAGCT | Antisense | 4496 |
| 241670_x_at | 479 | 825 | 354 | GGTAACCAGGGGAAGCTTGCGTGCC | Antisense | 4497 |
| 241671_x_at | 579 | 1 | 28 | ATTCTGAAGCCAGACCGTCTTTCCT | Antisense | 4498 |
| 241671_x_at | 716 | 993 | 223 | TCGCAGCTGGGCGAGGGGACTTGGA | Antisense | 4499 |
| 241671_x_at | 861 | 403 | 229 | CTGGGCGAGGGGACTTGGAGGACAG | Antisense | 4500 |
| 241671_x_at | 555 | 813 | 237 | GGGGACTTGGAGGACAGGGTGAAGC | Antisense | 4501 |
| 241671_x_at | 958 | 177 | 241 | ACTTGGAGGACAGGGTGAAGCTGCA | Antisense | 4502 |
| 241671_x_at | 834 | 951 | 256 | TGAAGCTGCAGAAGACCTGGGGTGG | Antisense | 4503 |
| 241671_x_at | 1116 | 501 | 260 | GCTGCAGAAGACCTGGGGTGGGATG | Antisense | 4504 |
| 241671_x_at | 425 | 1067 | 287 | TAGAGAGGACGCCAAGGACTGGGGA | Antisense | 4505 |
| 241671_x_at | 19 | 113 | 290 | AGAGGACGCCAAGGACTGGGGAAGG | Antisense | 4506 |
| 241671_x_at | 516 | 865 | 315 | GGAAGTTAGGAATACCTTACATCCA | Antisense | 4507 |
| 241671_x_at | 536 | 75 | 322 | AGGAATACCTTACATCCAATGCCCA | Antisense | 4508 |
| 243010_at | 200 | 597 | 608 | GAAATACACCCACTCTCTTGGAATA | Antisense | 4509 |
| 243010_at | 1151 | 57 | 633 | ATGACGTACCACTCAGTTGGACCCT | Antisense | 4510 |
| 243010_at | 856 | 619 | 652 | GACCCTCAAGAGTCACTGCTTTGTC | Antisense | 4511 |
| 243010_at | 506 | 417 | 704 | CGCACGCTTCCATTTGATGCATTTG | Antisense | 4512 |
| 243010_at | 1129 | 63 | 753 | ATGTCATTGTCCTTGAGACCCTACA | Antisense | 4513 |
| 243010_at | 296 | 639 | 767 | GAGACCCTACATGTGCAGTTTGGCT | Antisense | 4514 |
| 243010_at | 825 | 1097 | 840 | TTTCCTGCAGGCTTTTCCATGAGTA | Antisense | 4515 |
| 243010_at | 970 | 579 | 879 | GAACAAATCTGTATGGCTTTTCCCC | Antisense | 4516 |
| 243010_at | 483 | 767 | 943 | GTGAACTTGTCCTAGTATGCTTGCC | Antisense | 4517 |
| 243010_at | 1033 | 357 | 962 | CTTGCCTCACAAACGTTTTAGCCAT | Antisense | 4518 |
| 243010_at | 136 | 365 | 1119 | CTTAGCCTGACAGTGTCCTGTTCTC | Antisense | 4519 |
| 243502_at | 43 | 1127 | 166 | TTAATTTCTGTGAAGAGTGCCCCTG | Antisense | 4520 |
| 243502_at | 320 | 777 | 232 | GTGTCTGGATAACGCGTCAGCTTCT | Antisense | 4521 |
| 243502_at | 1061 | 679 | 239 | GATAACGCGTCAGCTTCTTAAAGTA | Antisense | 4522 |
| 243502_at | 865 | 1111 | 318 | TTATTCTCTTGCTAAAGTTGTAATT | Antisense | 4523 |
| 243502_at | 74 | 129 | 349 | AGAATTTTCCAGCTCTAACTGCCTT | Antisense | 4524 |
| 243502_at | 947 | 323 | 358 | CAGCTCTAACTGCCTTCTTAGTACA | Antisense | 4525 |
| 243502_at | 711 | 391 | 367 | CTGCCTTCTTAGTACATGTCTTTCT | Antisense | 4526 |
| 243502_at | 636 | 1133 | 372 | TTCTTAGTACATGTCTTTCTGCCTT | Antisense | 4527 |
| 243502_at | 218 | 367 | 386 | CTTTCTGCCTTTGAAGCATATGAGT | Antisense | 4528 |
| 243502_at | 401 | 941 | 406 | TGAGTTTGCCAAAGTCATTCTCCCC | Antisense | 4529 |
| 243502_at | 369 | 431 | 427 | CCCCTAATGACATATTGTGGACTTA | Antisense | 4530 |
| 244393_x_at | 880 | 421 | 132 | CCCAGCCGCTATAACTTTTAACAAT | Antisense | 4531 |
| 244393_x_at | 314 | 197 | 152 | ACAATTCCCATATGTCCTTTATTCC | Antisense | 4532 |
| 244393_x_at | 700 | 1077 | 162 | TATGTCCTTTATTCCACTAAGATGA | Antisense | 4533 |
| 244393_x_at | 464 | 59 | 183 | ATGAGTGCAGTATATATTTCCATCT | Antisense | 4534 |
| 244393_x_at | 489 | 23 | 196 | ATATTTCCATCTGTCCAAGGCTTCC | Antisense | 4535 |
| 244393_x_at | 796 | 795 | 270 | GGGCATTTGGGGAATCCAGGCTGTG | Antisense | 4536 |
| 244393_x_at | 1140 | 455 | 285 | CCAGGCTGTGATTCAGGGAAGTTCC | Antisense | 4537 |
| 244393_x_at | 597 | 257 | 303 | AAGTTCCAAGTGTCTGATGAAGTGT | Antisense | 4538 |
| 244393_x_at | 722 | 1155 | 328 | TTGTTTTACATCTTTGTGTCCCTTG | Antisense | 4539 |
| 244393_x_at | 247 | 453 | 348 | CCTTGCAGGTCTAGCACTGTGCTAT | Antisense | 4540 |
| 244393_x_at | 748 | 765 | 366 | GTGCTATGTAGGTAACATGTGCTCC | Antisense | 4541 |
| 91684_g_at | 134 | 141 | 67 | AGCTGCGCAAGATCCAGGCGCGGAT | Antisense | 4542 |
| 91684_g_at | 213 | 1139 | 99 | TTCGCGCAGGCTGACGGCTCGGCCT | Antisense | 4543 |
| 91684_g_at | 505 | 993 | 100 | TCGCGCAGGCTGACGGCTCGGCCTA | Antisense | 4544 |
| 91684_g_at | 811 | 341 | 105 | CAGGCTGACGGCTCGGCCTACATTG | Antisense | 4545 |
| 91684_g_at | 235 | 617 | 111 | GACGGCTCGGCCTACATTGAGCAGG | Antisense | 4546 |
| 91684_g_at | 776 | 383 | 116 | CTCGGCCTACATTGAGCAGGGCAAC | Antisense | 4547 |
| 91684_g_at | 67 | 449 | 121 | CCTACATTGAGCAGGGCAACACCAA | Antisense | 4548 |
| 91684_g_at | 114 | 3 | 126 | ATTGAGCAGGGCAACACCAAGGCAC | Antisense | 4549 |
| 91684_g_at | 765 | 629 | 129 | GAGCAGGGCAACACCAAGGCACTGG | Antisense | 4550 |
| 91684_g_at | 797 | 89 | 133 | AGGGCAACACCAAGGCACTGGCTGT | Antisense | 4551 |
| 91684_g_at | 1129 | 301 | 137 | CAACACCAAGGCACTGGCTGTGGTC | Antisense | 4552 |
| 91684_g_at | 769 | 189 | 139 | ACACCAAGGCACTGGCTGTGGTCTA | Antisense | 4553 |
| 91684_g_at | 976 | 295 | 143 | CAAGGCACTGGCTGTGGTCTACGGC | Antisense | 4554 |
| 91684_g_at | 319 | 75 | 145 | AGGCACTGGCTGTGGTCTACGGCCC | Antisense | 4555 |
| 91684_g_at | 1101 | 537 | 147 | GCACTGGCTGTGGTCTACGGCCCGC | Antisense | 4556 |
| 91684_g_at | 415 | 165 | 149 | ACTGGCTGTGGTCTACGGCCCGCAC | Antisense | 4557 |

TABLE 17
105 PS gene list probe sequences

| | | | | | | |
|---|---|---|---|---|---|---|
| | | | Probe | | | |
| Probe Set | | | Interrogation | | Target | |
| Name | Probe.X | Probe.Y | Position | Probe Sequence | Strandedness | SEQ ID NO: |

| | | | | | | |
|---|---|---|---|---|---|---|
| 1555852_at | 9 | 453 | 729 | CCATTCTGAGTACTTCTCCGCAAAC | Antisense | 4558 |
| 1555852_at | 567 | 615 | 770 | GACTGTTTTACATGAAGGGTGCAAA | Antisense | 4559 |
| 1555852_at | 99 | 1 | 811 | GAACCCTAGGGTGAAACACGTGACA | Antisense | 4560 |
| 1555852_at | 208 | 1089 | 849 | TATTGAATAGTCCTCTTCTCTACCC | Antisense | 4561 |
| 1555852_at | 446 | 255 | 926 | AAGAGATGAGCATTCAAGTCAGGCA | Antisense | 4562 |
| 1555852_at | 412 | 955 | 955 | TGAATTTGGGTCAAGGCTGCGCCAC | Antisense | 4563 |
| 1555852_at | 132 | 75 | 968 | AGGCTGCGCCACTCAAAAGCTATAT | Antisense | 4564 |
| 1555852_at | 158 | 25 | 989 | ATATGACCTCTATATGAGCAGCTTA | Antisense | 4565 |
| 1555852_at | 1121 | 1087 | 1012 | TATTCAACCTCTTTTAACCTCCATT | Antisense | 4566 |
| 1555852_at | 341 | 243 | 1027 | AACCTCCATTTTGTCATCTGTAGAA | Antisense | 4567 |
| 1555852_at | 310 | 1049 | 1058 | TAAATGCCTAGCTCAGAAGGATTCC | Antisense | 4568 |
| 1556340_at | 322 | 987 | 2212 | TCCCCCCTGGACAACTGTATACCCA | Antisense | 4569 |
| 1556340_at | 615 | 927 | 2227 | TGTATACCCAAGACAGTCCCCAGGG | Antisense | 4570 |
| 1556340_at | 78 | 723 | 2228 | GTATACCCAAGACAGTCCCCAGGGC | Antisense | 4571 |
| 1556340_at | 990 | 29 | 2230 | ATACCCAAGACAGTCCCCAGGGCCT | Antisense | 4572 |
| 1556340_at | 626 | 1039 | 2278 | TAAGCAGTCCAGACTCCAACCCCAA | Antisense | 4573 |
| 1556340_at | 830 | 807 | 2309 | GGGTCCATCATCCCCTATACCAGGG | Antisense | 4574 |
| 1556340_at | 947 | 793 | 2332 | GGGCTATCCCCTCAACTTGGAGGGC | Antisense | 4575 |
| 1556340_at | 229 | 881 | 2333 | GGCTATCCCCTCAACTTGGAGGGCT | Antisense | 4576 |
| 1556340_at | 1080 | 511 | 2334 | GCTATCCCCTCAACTTGGAGGGCTG | Antisense | 4577 |
| 1556340_at | 679 | 1079 | 2336 | TATCCCCTCAACTTGGAGGGCTGGC | Antisense | 4578 |
| 1556340_at | 871 | 425 | 2340 | CCCTCAACTTGGAGGGCTGGCCAAA | Antisense | 4579 |
| 1558034_s_at | 146 | 349 | 661 | CATCTACCCTGATAACACCACAGAT | Antisense | 4580 |
| 1558034_s_at | 344 | 51 | 711 | ATCCAGGAGAGCAGTATACATACAT | Antisense | 4581 |
| 1558034_s_at | 1145 | 31 | 726 | ATACATACATGTTGCTTGCCACTGA | Antisense | 4582 |
| 1558034_s_at | 808 | 769 | 785 | GTGACTAGGATTTACCATTCCCACA | Antisense | 4583 |
| 1558034_s_at | 12 | 355 | 800 | CATTCCCACATTGATGCTCCAAAAG | Antisense | 4584 |
| 1558034_s_at | 904 | 673 | 812 | GATGCTCCAAAAGATATTGCCTCAG | Antisense | 4585 |
| 1558034_s_at | 916 | 685 | 824 | GATATTGCCTCAGGACTCATCGGAC | Antisense | 4586 |
| 1558034_s_at | 456 | 377 | 839 | CTCATCGGACCTTTAATAATCTGTA | Antisense | 4587 |
| 1558034_s_at | 605 | 55 | 938 | ATGAAAATTTCAGCTGGTACCTAGA | Antisense | 4588 |
| 1558034_s_at | 328 | 221 | 973 | AAAACCTACTGCTCAGAACCAGAGA | Antisense | 4589 |
| 1558034_s_at | 7 | 683 | 1055 | GATACACTTTTGGAAGTCTCCCAGG | Antisense | 4590 |
| 1558290_a_at | 734 | 1111 | 2386 | TTTTGCATGTCTGACACCCATGACT | Antisense | 4591 |
| 1558290_a_at | 952 | 159 | 2432 | ACCCAGAAGCAATTCAGCCCAACAG | Antisense | 4592 |
| 1558290_a_at | 693 | 11 | 2443 | ATTCAGCCCAACAGGAGGACAGCTT | Antisense | 4593 |
| 1558290_a_at | 1152 | 659 | 2457 | GAGGACAGCTTCAACCCATTACGAT | Antisense | 4594 |
| 1558290_a_at | 483 | 1031 | 2467 | TCAACCCATTACGATTTCATCTCTG | Antisense | 4595 |
| 1558290_a_at | 605 | 13 | 2474 | ATTACGATTTCATCTCTGCCCCAAC | Antisense | 4596 |
| 1558290_a_at | 22 | 133 | 2504 | AGCAGCAAGCACCTGTTACCTGTCC | Antisense | 4597 |
| 1558290_a_at | 127 | 425 | 2552 | GCCTTTGAAAAATCCCTAACCTATG | Antisense | 4598 |
| 1558290_a_at | 104 | 203 | 2561 | AAATCCCTAACCTATGAGCTTTGAA | Antisense | 4599 |
| 1558290_a_at | 1127 | 425 | 2565 | CCCTAACCTATGAGCTTTGAATAAG | Antisense | 4600 |
| 1558290_a_at | 1011 | 109 | 2588 | AGATGAGTACGAACTTCATCGCCCA | Antisense | 4601 |
| 1559263_s_at | 319 | 517 | 5323 | GCTTCCTTTGAGAACAAGTTTGAGT | Antisense | 4602 |
| 1559263_s_at | 187 | 213 | 5361 | AAAGCTGTTTTATTTGATTCACTGT | Antisense | 4603 |
| 1559263_s_at | 693 | 499 | 5364 | GCTGTTTTATTTGATTCACTGTGAA | Antisense | 4604 |
| 1559263_s_at | 4 | 223 | 5407 | AAAAGTGAAAACACAGCACATTTCA | Antisense | 4605 |
| 1559263_s_at | 8 | 539 | 5422 | GCACATTTCAAGTGTAATCCCCTTT | Antisense | 4606 |
| 1559263_s_at | 1137 | 1123 | 5428 | TTCAAGTGTAATCCCCTTTGGAATT | Antisense | 4607 |
| 1559263_s_at | 1109 | 259 | 5459 | AAGTCTTAGTATATGTCTTTGTGAC | Antisense | 4608 |
| 1559263_s_at | 300 | 751 | 5473 | GTCTTTGTGACAGAAAAACATAGAT | Antisense | 4609 |
| 1559263_s_at | 900 | 65 | 5496 | ATGGGACAGGTAAATGAAATGTTCC | Antisense | 4610 |
| 1559263_s_at | 946 | 593 | 5511 | GAAATGTTCCTAGTTGCATAGCTGT | Antisense | 4611 |
| 1559263_s_at | 647 | 701 | 5516 | GTTCCTAGTTGCATAGCTGTTAACC | Antisense | 4612 |
| 1559584_a_at | 971 | 7 | 2313 | ATTCCTGGGCTCGAGAGAGCCACTC | Antisense | 4613 |
| 1559584_a_at | 263 | 775 | 2412 | GTGATTTTGCCATATTAGTTTACCA | Antisense | 4614 |
| 1559584_a_at | 986 | 1095 | 2417 | TTTGCCATATTAGTTTACCATCTGT | Antisense | 4615 |
| 1559584_a_at | 183 | 715 | 2429 | GTTTACCATCTGTACAATTATTTGC | Antisense | 4616 |
| 1559584_a_at | 262 | 455 | 2434 | CCATCTGTACAATTATTTGCTTTAT | Antisense | 4617 |
| 1559584_a_at | 10 | 351 | 2465 | CTTTAAATTGGCTCATTATTCACTT | Antisense | 4618 |
| 1559584_a_at | 197 | 293 | 2470 | AATTGGCTCATTATTCACTTAATGA | Antisense | 4619 |
| 1559584_a_at | 289 | 597 | 2493 | GAAATTATTGCAACAGGAATCTTTT | Antisense | 4620 |
| 1559584_a_at | 590 | 543 | 2502 | GCAACAGGAATCTTTTGTATCACTT | Antisense | 4621 |
| 1559584_a_at | 1055 | 553 | 2509 | GAATCTTTTGTATCACTTCTGTAAA | Antisense | 4622 |
| 1559584_a_at | 1061 | 751 | 2544 | GTCTCATTTGCCATCAATAGAAGGT | Antisense | 4623 |
| 1561042_at | 939 | 203 | 444 | AAATGCAGATAAAGGCTACCTCTGA | Antisense | 4624 |
| 1561042_at | 950 | 1047 | 453 | TAAAGGCTACCTCTGAATTCTCAAT | Antisense | 4625 |
| 1561042_at | 156 | 207 | 454 | AAAGGCTACCTCTGAATTCTCAATA | Antisense | 4626 |
| 1561042_at | 287 | 75 | 456 | AGGCTACCTCTGAATTCTCAATAGA | Antisense | 4627 |
| 1561042_at | 640 | 549 | 467 | GAATTCTCAATAGATTCATCATGTT | Antisense | 4628 |
| 1561042_at | 390 | 285 | 475 | AATAGATTCATCATGTTTGCTCTTA | Antisense | 4629 |
| 1561042_at | 971 | 691 | 479 | GATTCATCATGTTTGCTCTTAAGTG | Antisense | 4630 |
| 1561042_at | 921 | 1015 | 482 | TCATCATGTTTGCTCTTAAGTGTAG | Antisense | 4631 |
| 1561042_at | 76 | 719 | 489 | GTTTGCTCTTAAGTGTAGCTGTCCA | Antisense | 4632 |
| 1561042_at | 390 | 963 | 492 | TGCTCTTAAGTGTAGCTGTCCACAC | Antisense | 4633 |
| 1561042_at | 902 | 379 | 494 | CTCTTAAGTGTAGCTGTCCACACTG | Antisense | 4634 |
| 1562031_at | 1147 | 61 | 1892 | ATGTTCACTGTATGTGCCAAGCCTA | Antisense | 4635 |
| 1562031_at | 289 | 165 | 1898 | ACTGTATGTGCCAAGCCTAATATGA | Antisense | 4636 |
| 1562031_at | 811 | 761 | 1905 | GTGCCAAGCCTAATATGAGAGCTAT | Antisense | 4637 |
| 1562031_at | 374 | 1103 | 1933 | TTATAGAGTTTATGCTACAGCCCTA | Antisense | 4638 |
| 1562031_at | 1143 | 113 | 1937 | AGAGTTTATGCTACAGCCCTACCTT | Antisense | 4639 |
| 1562031_at | 529 | 189 | 1949 | ACAGCCCTACCTTCAGGAAACTTAT | Antisense | 4640 |
| 1562031_at | 743 | 77 | 1963 | AGGAAACTTATCTACTGGACAAACA | Antisense | 4641 |
| 1562031_at | 377 | 179 | 2022 | ACATTGTAATTATCTAGCATAGGCA | Antisense | 4642 |
| 1562031_at | 519 | 519 | 2038 | GCATAGGCAAATATAGACAGTAACA | Antisense | 4643 |
| 1562031_at | 968 | 229 | 2059 | AACAGACAGGTTTACAATTATTAAG | Antisense | 4644 |
| 1562031_at | 442 | 293 | 2073 | CAATTATTAAGAAAGGGCAGCCAGG | Antisense | 4645 |
| 1563497_at | 251 | 1063 | 4804 | TACCATAGCAAGTTCTTATCCTTTG | Antisense | 4646 |
| 1563497_at | 173 | 157 | 4861 | ACCTGTCACAGTAATATGCCAATTT | Antisense | 4647 |
| 1563497_at | 943 | 467 | 4878 | GCCAATTTTTGGTGTCGCTTTAGTA | Antisense | 4648 |
| 1563497_at | 834 | 749 | 4965 | GTCTTTCCTCAGTTGTCTCCTAAAA | Antisense | 4649 |
| 1563497_at | 555 | 677 | 5022 | GATCGTCTTGTGCAGCCCTTTAAAA | Antisense | 4650 |
| 1563497_at | 941 | 53 | 5048 | ATGCGTTTTGCATTCATGTCTTTGC | Antisense | 4651 |
| 1563497_at | 920 | 365 | 5088 | CTTTCATTTTGGGAGTGACTGTCAT | Antisense | 4652 |
| 1563497_at | 1079 | 189 | 5141 | ACACCAGGGCCAGATTGCGTGGGTT | Antisense | 4653 |
| 1563497_at | 610 | 965 | 5156 | TGCGTGGGTTCTAGCTAGGGACCTA | Antisense | 4654 |
| 1563497_at | 1053 | 141 | 5182 | AGCTGGTTACTTGATTTCTCTCTGT | Antisense | 4655 |
| 1563497_at | 93 | 1137 | 5197 | TTCTCTCTGTTCCATTTACTATCTG | Antisense | 4656 |
| 200904_at | 733 | 919 | 1987 | TGTGCCTTCATTCATGGGTTAATGG | Antisense | 4657 |
| 200904_at | 842 | 267 | 2015 | AATGGGTTATCACAGGAATGGGACT | Antisense | 4658 |
| 200904_at | 754 | 115 | 2063 | AGAGAACTGAGCTAGCATGCCCAGC | Antisense | 4659 |
| 200904_at | 296 | 421 | 2087 | CCCACAGAGAGCCTCCACTAGAGTG | Antisense | 4660 |
| 200904_at | 877 | 625 | 2095 | GAGCCTCCACTAGAGTGATGCTAAG | Antisense | 4661 |
| 200904_at | 590 | 749 | 2168 | GTCTAGTGTCTAGTGGATCCAGGCC | Antisense | 4662 |
| 200904_at | 756 | 839 | 2182 | GGATCCAGGCCACAGGAGAGAGTGC | Antisense | 4663 |
| 200904_at | 1010 | 237 | 2253 | AACTGTGGAGTCAGTGGCAGCATGC | Antisense | 4664 |
| 200904_at | 421 | 211 | 2292 | AAAGCTTTAGGCACCAGCCTGCAAC | Antisense | 4665 |
| 200904_at | 729 | 415 | 2322 | CGAGCAGCCACGTAGGCTGCACCCA | Antisense | 4666 |
| 200904_at | 806 | 451 | 2506 | CCTTCCCAGGCTGATCTGAGGTAAA | Antisense | 4667 |
| 200905_x_at | 720 | 213 | 1095 | AAAGCCTGAGACAGCTGCCTTGTGT | Antisense | 4668 |
| 200905_x_at | 1116 | 415 | 1121 | CGACTGAGATGCACAGCTGCCTTGT | Antisense | 4669 |
| 200905_x_at | 1111 | 503 | 1136 | GCTGCCTTGTGTGCGACTGAGATGC | Antisense | 4670 |
| 200905_x_at | 757 | 941 | 1153 | TGAGATGCAGGATTTCCTCACGCCT | Antisense | 4671 |
| 200905_x_at | 919 | 65 | 1183 | ATGTGTCTTAGGGGACTCTGGCTTC | Antisense | 4672 |
| 200905_x_at | 504 | 879 | 1202 | GGCTTCTCTTTTTGCAAGGGCCTCT | Antisense | 4673 |
| 200905_x_at | 369 | 999 | 1230 | TCTGTCTGTGTCCCTGTTAGCACAA | Antisense | 4674 |
| 200905_x_at | 608 | 115 | 1266 | AGAGAAACAGTCCACCTCTGTGTCT | Antisense | 4675 |
| 200905_x_at | 1142 | 913 | 1360 | TGGGCAGAGTGCGGCAGCTCATGCC | Antisense | 4676 |
| 200905_x_at | 917 | 771 | 1587 | GTGAGCCAGGATTGTGCGACTGCAC | Antisense | 4677 |
| 200905_x_at | 728 | 809 | 1620 | GGGTGACAGGGTGAAACGCCATCTC | Antisense | 4678 |
| 201009_s_at | 311 | 31 | 1461 | ATACTGAGGTGGATCCCTGCATCCT | Antisense | 4679 |
| 201009_s_at | 1005 | 427 | 1475 | CCCTGCATCCTCAACAACAATGTGC | Antisense | 4680 |
| 201009_s_at | 153 | 571 | 1520 | GAAGCAGCTTTACCTACTTGTTTCT | Antisense | 4681 |
| 201009_s_at | 57 | 365 | 1615 | CTTAGCCTCTGACTTCCTAATGTAG | Antisense | 4682 |
| 201009_s_at | 954 | 843 | 1639 | GGAGGTGGTCAGCAGGCAATCTCCT | Antisense | 4683 |
| 201009_s_at | 280 | 869 | 1653 | GGCAATCTCCTGGGCCTTAAAGGAT | Antisense | 4684 |
| 201009_s_at | 57 | 207 | 1671 | AAAGGATGCGGACTCATCCTCAGCC | Antisense | 4685 |
| 201009_s_at | 352 | 461 | 1694 | CCAGCGCCCATGTTGTGATACAGGG | Antisense | 4686 |
| 201009_s_at | 988 | 585 | 1749 | GAAAAACTCAGGCCCATCCATTTTC | Antisense | 4687 |
| 201009_s_at | 497 | 289 | 1789 | AATTGAGGCCTTTTCGATAGTTTCG | Antisense | 4688 |
| 201009_s_at | 245 | 205 | 1824 | AAATGGCCTCCTGGCGTAAGCTTTT | Antisense | 4689 |
| 201010_s_at | 1118 | 775 | 2116 | GTGTTCTCCTACTGCAAATATTTTC | Antisense | 4690 |
| 201010_s_at | 612 | 829 | 2153 | GGTTTTCTCTTCATGTAAGTCCTTG | Antisense | 4691 |
| 201010_s_at | 776 | 931 | 2196 | TGTTCTTAGCACTTTAATTCCTGTC | Antisense | 4692 |
| 201010_s_at | 968 | 499 | 2259 | GCTGAAACTGGTCTACTGTGTCTCT | Antisense | 4693 |
| 201010_s_at | 909 | 593 | 2373 | GAAAGGGTTGCTGCTGTCAGCCTTG | Antisense | 4694 |
| 201010_s_at | 538 | 301 | 2444 | CAACCCTGTGATCAGAACCTCCAAA | Antisense | 4695 |
| 201010_s_at | 777 | 107 | 2540 | AGATAGGGATATTGGCCCCTCACTG | Antisense | 4696 |
| 201010_s_at | 1097 | 313 | 2587 | CACTCTCAGCCATAGCACTTTGTTC | Antisense | 4697 |
| 201010_s_at | 814 | 175 | 2603 | ACTTTGTTCACTGTCCTGTGTCAGA | Antisense | 4698 |
| 201010_s_at | 668 | 917 | 2619 | TGTGTCAGAGCACTGAGCTCCACCC | Antisense | 4699 |
| 201010_s_at | 799 | 139 | 2634 | AGCTCCACCCTTTTCTGAGAGTTAT | Antisense | 4700 |
| 201891_s_at | 282 | 1105 | 535 | TTTATAATTCTACTTTGAGTGCTGT | Antisense | 4701 |
| 201891_s_at | 394 | 499 | 555 | GCTGTCTCCATGTTTGATGTATCTG | Antisense | 4702 |
| 201891_s_at | 1073 | 723 | 573 | GTATCTGAGCAGGTTGCTCCACAGG | Antisense | 4703 |
| 201891_s_at | 954 | 633 | 636 | GAGAATTCTCTTATCCAACATCAAC | Antisense | 4704 |
| 201891_s_at | 927 | 1135 | 641 | TTCTCTTATCCAACATCAACATCTT | Antisense | 4705 |
| 201891_s_at | 1077 | 347 | 660 | CATCTTGGTCAGATTTGAACTCTTC | Antisense | 4706 |
| 201891_s_at | 833 | 171 | 678 | ACTCTTCAATCTCTTGCACTCAAAG | Antisense | 4707 |
| 201891_s_at | 626 | 279 | 685 | AATCTCTTGCACTCAAAGCTTGTTA | Antisense | 4708 |
| 201891_s_at | 543 | 233 | 733 | AACTTCCAATTTACATACTCTGCTT | Antisense | 4709 |
| 201891_s_at | 868 | 691 | 840 | GATTCATATTTACTTCTTATACATT | Antisense | 4710 |
| 201891_s_at | 84 | 877 | 876 | GGCATGGTTGTGGTTAATCTGGTTT | Antisense | 4711 |
| 202546_at | 849 | 675 | 67 | GATCTGGTGCGGAACCTGCAAAGTG | Antisense | 4712 |
| 202546_at | 1110 | 421 | 119 | CCCAGAATGTGGAGCGGATCCTGGC | Antisense | 4713 |
| 202546_at | 90 | 585 | 151 | GAAAACTTGGAACATCTCCGCAACA | Antisense | 4714 |
| 202546_at | 1046 | 579 | 160 | GAACATCTCCGCAACAAGACAGAGG | Antisense | 4715 |
| 202546_at | 503 | 659 | 181 | GAGGATCTGGAAGCCACATCTGAGC | Antisense | 4716 |
| 202546_at | 497 | 245 | 191 | AAGCCACATCTGAGCACTTCAAGAC | Antisense | 4717 |
| 202546_at | 274 | 631 | 202 | GAGCACTTCAAGACGACATCGCAGA | Antisense | 4718 |
| 202546_at | 1001 | 617 | 213 | GACGACATCGCAGAAGGTGGCTCGA | Antisense | 4719 |
| 202546_at | 549 | 265 | 226 | AAGGTGGCTCGAAAATTCTGGTGGA | Antisense | 4720 |
| 202546_at | 152 | 671 | 261 | GATGATTGTCCTTATCTGCGTGATT | Antisense | 4721 |
| 202546_at | 672 | 463 | 320 | CCACTGGTGCCTTCTCTTAAAGTAA | Antisense | 4722 |
| 202688_at | 760 | 381 | 1181 | CTCTACCTCATATCAGTTTGCTAGC | Antisense | 4723 |
| 202688_at | 894 | 253 | 1216 | AAGACTGTCAGCTTCCAAACATTAA | Antisense | 4724 |
| 202688_at | 78 | 57 | 1240 | ATGCAATGGTTAACATCTTCTGTCT | Antisense | 4725 |
| 202688_at | 505 | 999 | 1258 | TCTGTCTTTATAATCTACTCCTTGT | Antisense | 4726 |
| 202688_at | 933 | 1035 | 1268 | TAATCTACTCCTTGTAAAGACTGTA | Antisense | 4727 |
| 202688_at | 971 | 123 | 1295 | AGAAAGCGCAACAATCCATCTCTCA | Antisense | 4728 |
| 202688_at | 450 | 51 | 1308 | ATCCATCTCTCAAGTAGTGTATCAC | Antisense | 4729 |
| 202688_at | 614 | 93 | 1323 | AGTGTATCACAGTAGTAGCCTCCAG | Antisense | 4730 |
| 202688_at | 623 | 1023 | 1329 | TCACAGTAGTAGCCTCCAGGTTTCC | Antisense | 4731 |
| 202688_at | 79 | 663 | 1388 | GAGGCACCACTAAAAGATCGCAGTT | Antisense | 4732 |
| 202688_at | 829 | 47 | 1404 | ATCGCAGTTTGCCTGGTGCAGTGGC | Antisense | 4733 |
| 203915_at | 1064 | 689 | 1973 | GATTATCAATTACCACACCATCTCC | Antisense | 4734 |
| 203915_at | 1124 | 569 | 2058 | GAAGCATGATTGGTGCCCAGTTAGC | Antisense | 4735 |
| 203915_at | 634 | 423 | 2073 | CCCAGTTAGCCTCTGCAGGATGTGG | Antisense | 4736 |
| 203915_at | 836 | 725 | 2131 | GTAGGAGAGGTTGTCTGTGGCCAGA | Antisense | 4737 |
| 203915_at | 555 | 153 | 2162 | ACCTATACTCACTTTCCCAAATTGA | Antisense | 4738 |
| 203915_at | 672 | 1151 | 2183 | TTGAATCACTGCTCACACTGCTGAT | Antisense | 4739 |
| 203915_at | 1055 | 1067 | 2212 | TAGAGTGCTGTCCGGTGGAGATCCC | Antisense | 4740 |
| 203915_at | 116 | 433 | 2235 | CCACCCGAACGTCTTATCTAATCAT | Antisense | 4741 |
| 203915_at | 256 | 41 | 2255 | ATCATGAAACTCCCTAGTTCCTTCA | Antisense | 4742 |
| 203915_at | 1156 | 701 | 2271 | GTTCCTTCATGTAACTTCCCTGAAA | Antisense | 4743 |
| 203915_at | 954 | 1093 | 2315 | TTTGAGAGTCTGTGACCCACTTACC | Antisense | 4744 |
| 204057_at | 184 | 293 | 2371 | AATTGTCGGATTTTCCATGTCCTCC | Antisense | 4745 |
| 204057_at | 401 | 243 | 2441 | AACCTCCCAGTGAAAGGGCAGCCTT | Antisense | 4746 |
| 204057_at | 679 | 593 | 2452 | GAAAGGGCAGCCTTCATTTTGAGAA | Antisense | 4747 |
| 204057_at | 934 | 801 | 2497 | GGGAGACAGCTCATCCAATCTCCCA | Antisense | 4748 |
| 204057_at | 1128 | 293 | 2512 | CAATCTCCCAAGTCTCATGGTGGAT | Antisense | 4749 |
| 204057_at | 440 | 771 | 2539 | GTGACTGTGAGAGTTTCCGGTTTAA | Antisense | 4750 |
| 204057_at | 600 | 245 | 2572 | AAGCCAGATATGCCTGTTTCCTTTT | Antisense | 4751 |
| 204057_at | 32 | 787 | 2612 | GTGGAGGGGACAGTCAGACCCAGAG | Antisense | 4752 |
| 204057_at | 554 | 619 | 2628 | GACCCAGAGGTCCTTTACGTGTGGA | Antisense | 4753 |
| 204057_at | 1009 | 79 | 2673 | AGGAGAGGACCAGGGGACGTGGCTT | Antisense | 4754 |
| 204057_at | 840 | 223 | 2768 | AACAATAGGCTTGAATCTCCAATTC | Antisense | 4755 |
| 204078_at | 1122 | 233 | 2016 | AACTTAAGATGGACACAGCTGACTG | Antisense | 4756 |
| 204078_at | 339 | 111 | 2022 | AGATGGACACAGCTGACTGGACCCC | Antisense | 4757 |
| 204078_at | 40 | 313 | 2091 | CACTTCTATGTCTCTGGACCACAGG | Antisense | 4758 |
| 204078_at | 125 | 871 | 2137 | GGCAAGTGGGCTGATGGGGTCCGCC | Antisense | 4759 |
| 204078_at | 895 | 751 | 2192 | GTCTCCTGGACAAGGAGTTTCCAGC | Antisense | 4760 |
| 204078_at | 470 | 439 | 2196 | CCTGGACAAGGAGTTTCCAGCTGCT | Antisense | 4761 |
| 204078_at | 460 | 885 | 2221 | GGCTGGAGTCTCAGGCCAAATTGCA | Antisense | 4762 |
| 204078_at | 55 | 73 | 2233 | AGGCCAAATTGCAGAGGGTCCTCCA | Antisense | 4763 |
| 204078_at | 1110 | 337 | 2256 | CAGGGTCCTGAAGAGCACTGGACTA | Antisense | 4764 |
| 204078_at | 1062 | 441 | 2262 | CCTGAAGAGCACTGGACTAAGAGTC | Antisense | 4765 |
| 204078_at | 250 | 173 | 2277 | ACTAAGAGTCTAGTGGTTCCAGGGC | Antisense | 4766 |
| 204116_at | 475 | 1137 | 867 | TTCTGGCTGGAACGGACGATGCCCC | Antisense | 4767 |
| 204116_at | 606 | 821 | 917 | GGATCTTGTTACTGAATACCACGGG | Antisense | 4768 |
| 204116_at | 110 | 817 | 960 | GGTGTGTCTAAGGGACTGGCTGAGA | Antisense | 4769 |
| 204116_at | 902 | 173 | 997 | ACTACAGTGAACGACTCTGCCTCGT | Antisense | 4770 |
| 204116_at | 121 | 613 | 1009 | GACTCTGCCTCGTCAGTGAGATTCC | Antisense | 4771 |
| 204116_at | 998 | 707 | 1111 | GTTACACCCTAAAGCCTGAAACCTG | Antisense | 4772 |
| 204116_at | 845 | 241 | 1136 | AACCCCAATCCTCTGACAGAAGAAC | Antisense | 4773 |
| 204116_at | 309 | 819 | 1166 | GGTCCTGTAGCCCTAAGTGGTACTA | Antisense | 4774 |
| 204116_at | 1123 | 781 | 1182 | GTGGTACTAACTTTCCTTCATTCAA | Antisense | 4775 |
| 204116_at | 877 | 883 | 1242 | GGCTGATTTGGAATTTTGTGCCCCC | Antisense | 4776 |
| 204116_at | 453 | 285 | 1253 | AATTTTGTGCCCCCATGTAAGCACC | Antisense | 4777 |
| 204233_s_at | 172 | 543 | 1157 | GCAAACATCCGGAAGTATCCCACCA | Antisense | 4778 |
| 204233_s_at | 743 | 1079 | 1172 | TATCCCACCAAGAAACAACAGCTCC | Antisense | 4779 |
| 204233_s_at | 950 | 1085 | 1201 | TATTTCCAGTTACTTGCCTGCATTC | Antisense | 4780 |
| 204233_s_at | 431 | 1035 | 1294 | TAATAGGTTTGCCCTTGCATCTCAT | Antisense | 4781 |
| 204233_s_at | 378 | 801 | 1327 | GGGACTGTGGTCCATTGTACAAGCC | Antisense | 4782 |
| 204233_s_at | 60 | 549 | 1367 | GAATTTGGGTACATGGACTACGCCC | Antisense | 4783 |
| 204233_s_at | 307 | 857 | 1381 | GGACTACGCCCAAGCAAGGTTTGAT | Antisense | 4784 |
| 204233_s_at | 803 | 565 | 1426 | GAAGCTTGGGGTGTGACTGTGGGGA | Antisense | 4785 |
| 204233_s_at | 152 | 155 | 1462 | ACCTCATCACTGGACTGCATGGGGA | Antisense | 4786 |
| 204233_s_at | 742 | 789 | 1530 | GTGGCAGGAGGCTTTGGGTGGCTCA | Antisense | 4787 |
| 204233_s_at | 936 | 1091 | 1542 | TTTGGGTGGCTCACTACTGAACACA | Antisense | 4788 |
| 204661_at | 99 | 189 | 14 | ACAGCCACGAAGATCCTACCAAAAT | Antisense | 4789 |
| 204661_at | 730 | 465 | 32 | CCAAAATGAAGCGCTTCCTCTTCCT | Antisense | 4790 |
| 204661_at | 1029 | 347 | 66 | CATCAGCCTCCTGGTTATGGTACAG | Antisense | 4791 |
| 204661_at | 518 | 827 | 84 | GGTACAGATACAAACTGGACTCTCA | Antisense | 4792 |
| 204661_at | 966 | 611 | 101 | GACTCTCAGGACAAAACGACACCAG | Antisense | 4793 |
| 204661_at | 445 | 523 | 145 | GCATCCAGCAGCATGAGCGGAGGCA | Antisense | 4794 |
| 204661_at | 692 | 791 | 184 | GTGGCCAATGCCATAATCCACCTCT | Antisense | 4795 |
| 204661_at | 635 | 105 | 217 | AGTTGAGGTGACACGTCTCAGCCTT | Antisense | 4796 |
| 204661_at | 553 | 471 | 266 | GCCACCATCACTCGCAAGAGAATCC | Antisense | 4797 |
| 204661_at | 162 | 1161 | 300 | TTGGGAGGGGTTGATGCCAGACATC | Antisense | 4798 |
| 204661_at | 357 | 811 | 377 | GGGGGTAATGATGTAGGGGCCAAGC | Antisense | 4799 |
| 204724_s_at | 432 | 321 | 1896 | CACCAGCAAGGACGGCCAGGACGGT | Antisense | 4800 |
| 204724_s_at | 538 | 145 | 2007 | AGCCTGCCAAGGAGCCGTGTTAGGA | Antisense | 4801 |
| 204724_s_at | 1011 | 595 | 2043 | GAAATCAGGCTCTCGAAGCTCATAA | Antisense | 4802 |
| 204724_s_at | 984 | 569 | 2081 | GAAGCAAGTGACAAGGACGCCCGAA | Antisense | 4803 |
| 204724_s_at | 4 | 619 | 2096 | GACGCCCGAAGCACAGTGGACGGTC | Antisense | 4804 |
| 204724_s_at | 198 | 855 | 2113 | GGACGGTCATGAAGGAGCGGGGGTG | Antisense | 4805 |
| 204724_s_at | 188 | 761 | 2153 | GTCCAGGAGAGGGAGCGCCCCTGGC | Antisense | 4806 |
| 204724_s_at | 537 | 883 | 2232 | GGCTGTCGCCTGACAGCATACCTCA | Antisense | 4807 |
| 204724_s_at | 1032 | 519 | 2247 | GCATACCTCAAAAGGCCCTAGCTAA | Antisense | 4808 |
| 204724_s_at | 790 | 833 | 2323 | GGTTGTGTACAACTCCACGAGGTGA | Antisense | 4809 |
| 204724_s_at | 617 | 1045 | 2383 | TAAAGACTATGATCTCATCCCAATA | Antisense | 4810 |
| 205159_at | 313 | 689 | 4290 | GATTTACAAAGGTCCTCCCATTGCA | Antisense | 4811 |
| 205159_at | 399 | 1099 | 4346 | TTTCTAGTTCATTTTGTGTTTCCAA | Antisense | 4812 |
| 205159_at | 861 | 841 | 4407 | GGATGTGAGACTGAGGTGCCTTTTG | Antisense | 4813 |
| 205159_at | 984 | 821 | 4421 | GGTGCCTTTTGGTACTGAAATTCTT | Antisense | 4814 |
| 205159_at | 1153 | 595 | 4437 | GAAATTCTTTTTCCATGTACCTGAA | Antisense | 4815 |
| 205159_at | 486 | 1083 | 4496 | TATACTTTATTGGTCCCTAGGCTTC | Antisense | 4816 |
| 205159_at | 840 | 75 | 4514 | AGGCTTCCTATTTTGTTACCTTGCT | Antisense | 4817 |
| 205159_at | 225 | 1121 | 4529 | TTACCTTGCTTTCTCTATGGCATCC | Antisense | 4818 |
| 205159_at | 495 | 897 | 4546 | TGGCATCCACCATTTTGATTGTTCT | Antisense | 4819 |
| 205159_at | 64 | 721 | 4606 | GTATTCTCGTTACTTTTGCTCTTAA | Antisense | 4820 |
| 205159_at | 1047 | 729 | 4733 | GTAGAATCCTCTGTTCATAATGAAC | Antisense | 4821 |
| 205204_at | 363 | 109 | 167 | AGATCCGAGTGCACTCGCGAGGCAA | Antisense | 4822 |
| 205204_at | 229 | 463 | 200 | CCACCGGTCACTTCATGGGCAAGAA | Antisense | 4823 |
| 205204_at | 297 | 801 | 281 | GGGACCAGCGACTGCAGCTGAGTCA | Antisense | 4824 |
| 205204_at | 580 | 649 | 300 | GAGTCATGATCTGCTCGGAATCCTC | Antisense | 4825 |
| 205204_at | 324 | 963 | 311 | TGCTCGGAATCCTCCTGCTAAAGAA | Antisense | 4826 |
| 205204_at | 608 | 535 | 364 | GCACCCCAAATCCAGTACAGGAGGC | Antisense | 4827 |
| 205204_at | 882 | 1073 | 424 | TAGGGGCAGACACAACAGCGTGGCT | Antisense | 4828 |
| 205204_at | 829 | 141 | 440 | AGCGTGGCTTAGATTGTGCCCACCC | Antisense | 4829 |
| 205204_at | 311 | 763 | 455 | GTGCCCACCCAGGGAAGGTGCTGAA | Antisense | 4830 |
| 205204_at | 1159 | 951 | 476 | TGAATGGGACCCTGTTGATGGCCCC | Antisense | 4831 |
| 205204_at | 541 | 279 | 525 | AATCTCTGTTACTCCATTACTGTGA | Antisense | 4832 |
| 205419_at | 665 | 323 | 1055 | CAGCCCCTGAAGAAAATTCACGTGA | Antisense | 4833 |
| 205419_at | 139 | 671 | 1095 | GATGATGATACATTCCAAGTCTTCA | Antisense | 4834 |
| 205419_at | 477 | 599 | 1171 | GACAAACTTTGCAGGACTTCCCTTA | Antisense | 4835 |
| 205419_at | 288 | 525 | 1181 | GCAGGACTTCCCTTATAAAGCAAAA | Antisense | 4836 |
| 205419_at | 1029 | 705 | 1211 | GTTCAGCTTCCAATTAGTATTCTTT | Antisense | 4837 |
| 205419_at | 465 | 1085 | 1236 | TATATTTCTTTCATTGGGCGCTTTC | Antisense | 4838 |
| 205419_at | 228 | 365 | 1256 | CTTTCCCATCTCCAACTCGGAAGTA | Antisense | 4839 |
| 205419_at | 104 | 565 | 1275 | GAAGTAAGCCCAAGAGAACAACATA | Antisense | 4840 |
| 205419_at | 952 | 551 | 1361 | GAATACACCAAAAGGAGGCGCTCTT | Antisense | 4841 |
| 205419_at | 451 | 1 | 1526 | AAATCCAAATTTCTCTCAATGTTAG | Antisense | 4842 |
| 205419_at | 469 | 649 | 1550 | GATTTAATTCCCTCAATAACACCAA | Antisense | 4843 |
| 205668_at | 791 | 863 | 6476 | GGAACTCTAAACCTTGTGATGACTA | Antisense | 4844 |
| 205668_at | 475 | 37 | 6550 | ATCACTTTAAGTTTTGACACCTAGA | Antisense | 4845 |
| 205668_at | 859 | 749 | 6580 | GTCTTAGTAATAGCATCCACTGGAA | Antisense | 4846 |
| 205668_at | 914 | 1021 | 6623 | TCAGCATTTAACTTACATTTGTACT | Antisense | 4847 |
| 205668_at | 496 | 1105 | 6739 | TTTATATAGTCTACAGTTGTCCTGA | Antisense | 4848 |
| 205668_at | 868 | 105 | 6753 | AGTTGTCCTGATTTCTTATTGAATT | Antisense | 4849 |
| 205668_at | 328 | 551 | 6773 | GAATTTGTTAGACTAGTTCTCTTGT | Antisense | 4850 |
| 205668_at | 85 | 705 | 6788 | GTTCTCTTGTCTTGTGATCTGTGTA | Antisense | 4851 |
| 205668_at | 695 | 1103 | 6817 | TTTAGTCACTAAGACTTTCCTCCAA | Antisense | 4852 |
| 205668_at | 422 | 1097 | 6832 | TTTCCTCCAAGAACTAAGCCAACTT | Antisense | 4853 |
| 205668_at | 261 | 165 | 6869 | ACGGCTGTATATAATGGTGATGTCA | Antisense | 4854 |
| 205696_s_at | 858 | 1143 | 2023 | TTCCTGTTCTCTTGTATAGCTGAAA | Antisense | 4855 |
| 205696_s_at | 927 | 703 | 2028 | GTTCTCTTGTATAGCTGAAATTCCA | Antisense | 4856 |
| 205696_s_at | 669 | 597 | 2044 | GAAATTCCAGTTTAGGAGCTCAGTT | Antisense | 4857 |
| 205696_s_at | 1014 | 635 | 2069 | GAGAAACAGTTCCATTCAACTGGAA | Antisense | 4858 |
| 205696_s_at | 17 | 951 | 2146 | TGAAAAACCTGATGCAGTGCTCCAT | Antisense | 4859 |
| 205696_s_at | 491 | 799 | 2209 | GGGACAGTTTGTAACTTGGGCTGTA | Antisense | 4860 |
| 205696_s_at | 666 | 1041 | 2344 | TAACTCCCATATCTCCTTTAATGAC | Antisense | 4861 |
| 205696_s_at | 299 | 425 | 2349 | CCCATATCTCCTTTAATGACATTGA | Antisense | 4862 |
| 205696_s_at | 247 | 5 | 2401 | ATTGATGCCAAGCTTTTTTGCCACA | Antisense | 4863 |
| 205696_s_at | 68 | 937 | 2403 | TGATGCCAAGCTTTTTTGCCACAAA | Antisense | 4864 |
| 205696_s_at | 1048 | 793 | 2447 | GGGCTTTGTGGAAACAGCTGGTACT | Antisense | 4865 |
| 205758_at | 463 | 323 | 1584 | CAGCCCTTGCATTGCAGAGGGGCCC | Antisense | 4866 |
| 205758_at | 1044 | 947 | 1610 | TGAAAGAGGACAGGCTACCCCTTTA | Antisense | 4867 |
| 205758_at | 210 | 217 | 1663 | AAACTAAGGCCCTCTTGAATCTCTG | Antisense | 4868 |
| 205758_at | 1134 | 681 | 1695 | GATACAAACATGTTCCTGGGATCAC | Antisense | 4869 |
| 205758_at | 390 | 123 | 1743 | AGACAATTGTTGGAGAGCCCCTCAC | Antisense | 4870 |
| 205758_at | 338 | 59 | 1804 | ATGAGGCAGACCTGACTCTCTTAAG | Antisense | 4871 |
| 205758_at | 858 | 237 | 1844 | AACTGCTGTCCCAAACATGCACTTC | Antisense | 4872 |
| 205758_at | 774 | 217 | 1856 | AAACATGCACTTCCTTGCTTAAGGT | Antisense | 4873 |
| 205758_at | 546 | 69 | 1881 | ATGGTACAAGCAATGCCTGCCCATT | Antisense | 4874 |
| 205758_at | 190 | 1077 | 1982 | TATGGTGTACATTCTTCCTGATTAT | Antisense | 4875 |
| 205758_at | 1084 | 359 | 1995 | CTTCCTGATTATTTTCTACACATAC | Antisense | 4876 |
| 205890_s_at | 632 | 625 | 195 | GATCTTAAAGCCACGGAGAAGCCTC | Antisense | 4877 |
| 205890_s_at | 247 | 969 | 293 | TGCCCTTGTTTCTTGTGGAGTCAGG | Antisense | 4878 |
| 205890_s_at | 584 | 59 | 320 | ATGAGGCAAAGAGGCACCTCCTCCA | Antisense | 4879 |
| 205890_s_at | 578 | 561 | 350 | GAAGGTCCAGCTCAGTGGCACAAGT | Antisense | 4880 |
| 205890_s_at | 767 | 615 | 396 | GACGGGTATAATCCCTGAGACCCAG | Antisense | 4881 |
| 205890_s_at | 615 | 619 | 414 | GACCCAGATTGTGACTTGCAATGGA | Antisense | 4882 |
| 205890_s_at | 783 | 113 | 481 | AGAAAGGGCAACTTACTCTTCCTGG | Antisense | 4883 |
| 205890_s_at | 67 | 173 | 495 | ACTCTTCCTGGCATCTTATTGTATT | Antisense | 4884 |
| 205890_s_at | 814 | 279 | 578 | AATCTCTTACTCAACGAACACATCT | Antisense | 4885 |
| 205890_s_at | 573 | 349 | 598 | CATCTTCTGATGATTTCCCAAAATT | Antisense | 4886 |
| 205890_s_at | 415 | 7 | 678 | ATTGCCCAACTCTATGTTTCTTTGA | Antisense | 4887 |
| 206082_at | 159 | 953 | 2011 | TGAAGGATGGTGACTGCGCCATGGC | Antisense | 4888 |
| 206082_at | 303 | 981 | 2053 | TCCTTTCCTGTGGAGGCTCCACTCA | Antisense | 4889 |
| 206082_at | 752 | 981 | 2087 | TCCTCCTATGTCACCTAGAGTGTGG | Antisense | 4890 |
| 206082_at | 638 | 235 | 2150 | AACTCAAACAGGCTCACCAGGCAGT | Antisense | 4891 |
| 206082_at | 233 | 673 | 2202 | GATGCAACAGTTTGTCTTCACATTG | Antisense | 4892 |
| 206082_at | 127 | 1127 | 2218 | TTCACATTGGAAGGACACCCCTGGA | Antisense | 4893 |
| 206082_at | 890 | 173 | 2254 | ACTAGACCTGTAAAACTTCACTGCA | Antisense | 4894 |
| 206082_at | 286 | 469 | 2282 | GCCACTTCTGAATCTCTGTAAGGTT | Antisense | 4895 |
| 206082_at | 952 | 1119 | 2340 | TTACCAAAGCCTCTAGTGTACCGTC | Antisense | 4896 |
| 206082_at | 861 | 977 | 2438 | TCCAGGTTCTCCAGATCTCTTAAGA | Antisense | 4897 |
| 206082_at | 811 | 249 | 2459 | AAGACTGTACTATAGAGGCCTGGGG | Antisense | 4898 |
| 206134_at | 437 | 367 | 1600 | CTTTCTATATTGTTATCAGTCCAGG | Antisense | 4899 |
| 206134_at | 424 | 607 | 1650 | GACATTGGCTCTTTGTTTAGGCCTA | Antisense | 4900 |
| 206134_at | 26 | 551 | 1725 | GAATTTGTGACTTAGTTCTGCCCTT | Antisense | 4901 |
| 206134_at | 512 | 591 | 1762 | GAAAGCAGTCTTCCATCAAATCACC | Antisense | 4902 |
| 206134_at | 398 | 275 | 1791 | AATGCACGGCTAAACTATTCAGAGT | Antisense | 4903 |
| 206134_at | 1079 | 259 | 1841 | AAGTACTATGCTTTAATGCTTCTTT | Antisense | 4904 |
| 206134_at | 443 | 517 | 1858 | GCTTCTTTCATCTTACTAGTATGGC | Antisense | 4905 |
| 206134_at | 791 | 1077 | 1953 | TATGTTATTCCTCTGTGTTCACTTC | Antisense | 4906 |
| 206134_at | 539 | 177 | 1973 | ACTTCGCCTTGCTCTTGAAAGTGCA | Antisense | 4907 |
| 206134_at | 1134 | 721 | 2055 | GTATATCCTATACACACATCTCCTT | Antisense | 4908 |
| 206134_at | 103 | 943 | 2104 | TGAGAAGTCTACATTGCTTACATTT | Antisense | 4909 |
| 206540_at | 951 | 837 | 1035 | GGATCAATGGGTTTAACTTGGGCCG | Antisense | 4910 |
| 206540_at | 542 | 175 | 1050 | ACTTGGGCCGGTACTGGACAAAGCA | Antisense | 4911 |
| 206540_at | 1042 | 599 | 1066 | GACAAAGCAGGGGCCACAACAGACC | Antisense | 4912 |
| 206540_at | 602 | 467 | 1100 | CCAAGATTCCTGCTGTTTCCTAGGG | Antisense | 4913 |
| 206540_at | 228 | 813 | 1122 | GGGGAGCCCTCAACAAAATTACATT | Antisense | 4914 |
| 206540_at | 450 | 235 | 1152 | AACTAGAAGATGTACCTCTCCAGCC | Antisense | 4915 |
| 206540_at | 479 | 975 | 1170 | TCCAGCCCCAAGTCCAATTTTTGGA | Antisense | 4916 |
| 206540_at | 663 | 477 | 1198 | GCCTATCCTCAATAGCACTAGTACT | Antisense | 4917 |
| 206540_at | 528 | 313 | 1213 | CACTAGTACTTTGCACAGGACACAT | Antisense | 4918 |
| 206540_at | 343 | 193 | 1232 | ACACATATCAATTCCCTTTCAGCTG | Antisense | 4919 |
| 206540_at | 626 | 933 | 1255 | TGATACACTGAGTGCCTCTGAACCA | Antisense | 4920 |
| 206574_s_at | 471 | 561 | 426 | GAAGGATGGCATCACCGTTGTGGAC | Antisense | 4921 |
| 206574_s_at | 361 | 897 | 432 | TGGCATCACCGTTGTGGACTGGCCG | Antisense | 4922 |
| 206574_s_at | 1111 | 615 | 448 | GACTGGCCGTTTGACGATGGGGCGC | Antisense | 4923 |
| 206574_s_at | 1124 | 827 | 486 | GGTAGTGGAAGACTGGCTGAGCCTG | Antisense | 4924 |
| 206574_s_at | 887 | 349 | 669 | CATCAACAGCAAGCAGCTCACCTAC | Antisense | 4925 |
| 206574_s_at | 830 | 1023 | 686 | TCACCTACCTGGAGAAATACCGGCC | Antisense | 4926 |
| 206574_s_at | 596 | 331 | 715 | CAGAGGCTGCGGTTCAAAGACCCAC | Antisense | 4927 |
| 206574_s_at | 384 | 541 | 744 | GCACAAGACCCGGTGCTGCGTTATG | Antisense | 4928 |
| 206574_s_at | 249 | 493 | 761 | GCGTTATGTAGCTCAGGACCTTGGC | Antisense | 4929 |
| 206574_s_at | 117 | 793 | 787 | GGGCCTGGTCGTCATGTAGGTCAGG | Antisense | 4930 |
| 206574_s_at | 1126 | 443 | 910 | CCTCCGTGCACTTGTGTCCGAGGAG | Antisense | 4931 |
| 206666_at | 531 | 211 | 471 | AAACCTCTCTTAGATCTGGAACCAA | Antisense | 4932 |
| 206666_at | 1105 | 147 | 517 | AGCCACCGATCCAGATTCATTAAGA | Antisense | 4933 |
| 206666_at | 321 | 1131 | 532 | TTCATTAAGACCTTCTGACACCCTG | Antisense | 4934 |
| 206666_at | 404 | 191 | 549 | ACACCCTGCGAGAAGTCACTGTTAC | Antisense | 4935 |
| 206666_at | 594 | 813 | 696 | GGGGCCCCTTGATCTGTAAAGGTGT | Antisense | 4936 |
| 206666_at | 586 | 207 | 713 | AAAGGTGTCTTCCACGCTATAGTCT | Antisense | 4937 |
| 206666_at | 673 | 785 | 753 | GTGGTGTTGCCACAAAGCCTGGAAT | Antisense | 4938 |
| 206666_at | 972 | 867 | 773 | GGAATCTACACCCTGTTAACCAAGA | Antisense | 4939 |
| 206666_at | 335 | 905 | 809 | TGGATCAAAAGCAACCTTGTCCCGC | Antisense | 4940 |
| 206666_at | 590 | 757 | 827 | GTCCCGCCTCATACAAATTAAGTTA | Antisense | 4941 |
| 206666_at | 448 | 1075 | 935 | TATGGGGTCCATTTTTGCACTTGTA | Antisense | 4942 |
| 207175_at | 334 | 199 | 3936 | AAATAACATACGCACTCAACTTCCT | Antisense | 4943 |
| 207175_at | 707 | 355 | 4062 | CTTGACAGACTCTCGTTCTGTGGCC | Antisense | 4944 |
| 207175_at | 793 | 169 | 4119 | ACTGCTACCTCTACCATGAGCAATT | Antisense | 4945 |
| 207175_at | 446 | 155 | 4125 | ACCTCTACCATGAGCAATTCTCCTG | Antisense | 4946 |
| 207175_at | 845 | 1025 | 4232 | TCACCACATTCGTCAGGTTGGTTTC | Antisense | 4947 |
| 207175_at | 703 | 349 | 4321 | CATCACACCTGGTCAACTTTCTTTT | Antisense | 4948 |
| 207175_at | 126 | 25 | 4364 | ATATCTTTTTCCATCATGTTACTTT | Antisense | 4949 |
| 207175_at | 123 | 273 | 4415 | AATGTGTTTCTTACAGACTGCATGT | Antisense | 4950 |
| 207175_at | 860 | 187 | 4427 | ACAGACTGCATGTAGTTGGGTATAA | Antisense | 4951 |
| 207175_at | 1154 | 695 | 4441 | GTTGGGTATAATTTTTATCCAGTCT | Antisense | 4952 |
| 207175_at | 1147 | 285 | 4469 | AATATCTGTCTTTTAATTGGTGTTT | Antisense | 4953 |
| 207651_at | 757 | 1147 | 828 | TTGCCTTGTAATTCGACAGCTCTAC | Antisense | 4954 |
| 207651_at | 1133 | 175 | 906 | ACTTTTAGTGACCACGGGCTACATC | Antisense | 4955 |
| 207651_at | 103 | 795 | 921 | GGGCTACATCATATGCTTTGTTCCT | Antisense | 4956 |
| 207651_at | 23 | 31 | 968 | ATACCCTCAGCCAGACAGAAGTCAT | Antisense | 4957 |
| 207651_at | 997 | 563 | 985 | GAAGTCATAACTGATTGCTCAACCA | Antisense | 4958 |
| 207651_at | 1084 | 961 | 1000 | TGCTCAACCAGGATTTCACTCTTCA | Antisense | 4959 |
| 207651_at | 572 | 245 | 1025 | AAGCCAAAGAGGCTACACTGCTCCT | Antisense | 4960 |
| 207651_at | 1026 | 961 | 1066 | TGCTTTGATCCTATCCTGTACTATC | Antisense | 4961 |
| 207651_at | 257 | 211 | 1099 | AAAGCATTCCGCTCAAAGGTCACTG | Antisense | 4962 |
| 207651_at | 1005 | 1027 | 1118 | TCACTGAGACTTTTGCCTCACCTAA | Antisense | 4963 |
| 207651_at | 800 | 959 | 1208 | TGCTACCAATTCTGGCCTTACTGGA | Antisense | 4964 |
| 208296_x_at | 1080 | 1151 | 1417 | TTGAGTTCTCCTTTTAAGTACCAAT | Antisense | 4965 |
| 208296_x_at | 428 | 811 | 1469 | GGTGTGTCATTGCCTTGAAATGCTT | Antisense | 4966 |
| 208296_x_at | 537 | 595 | 1485 | GAAATGCTTGCTTAGGGCTTCTTTT | Antisense | 4967 |
| 208296_x_at | 564 | 1073 | 1497 | TAGGGCTTCTTTTATGTTATCTTAA | Antisense | 4968 |
| 208296_x_at | 1109 | 261 | 1523 | AAGTGCTGGTGAATTTTCCATTTTT | Antisense | 4969 |
| 208296_x_at | 683 | 1145 | 1538 | TTCCATTTTTTACATCCATTTCACA | Antisense | 4970 |
| 208296_x_at | 857 | 593 | 1627 | GAAAGGTAACAATCTTCATTCTACA | Antisense | 4971 |
| 208296_x_at | 713 | 275 | 1727 | AATGCGTGCGTTTCAGTGTTTAAGA | Antisense | 4972 |
| 208296_x_at | 1046 | 297 | 1843 | CAAGCAGTTTGTGTGGTGTTTCTAT | Antisense | 4973 |
| 208296_x_at | 1051 | 201 | 1928 | AAATCTGCTGGCCAGCTATGTCCTC | Antisense | 4974 |
| 208296_x_at | 1016 | 1071 | 1953 | TAGGAAATGACAGACCCAACCACCA | Antisense | 4975 |
| 209606_at | 595 | 493 | 1164 | GAATTGCAAAACTGACATCCCATTT | Antisense | 4976 |
| 209606_at | 140 | 169 | 1174 | ACTGACATCCCATTTCACAGCAATA | Antisense | 4977 |
| 209606_at | 1008 | 309 | 1189 | CACAGCAATAGTGACCTTTATTTAA | Antisense | 4978 |
| 209606_at | 778 | 697 | 1217 | GTTGTGTTATAGTTTATGCTTCTTA | Antisense | 4979 |
| 209606_at | 422 | 1099 | 1249 | TTTCAACCTAAACAGCCAATTTCTA | Antisense | 4980 |
| 209606_at | 735 | 189 | 1260 | ACAGCCAATTTCTAAGCAGACAGGA | Antisense | 4981 |
| 209606_at | 240 | 1087 | 1369 | TATTTTCTAGATTATCCCTGTGAAT | Antisense | 4982 |
| 209606_at | 292 | 201 | 1485 | AAATCTTAGGTTTGCTTATGCCCAG | Antisense | 4983 |
| 209606_at | 775 | 499 | 1590 | GCTGTTTCTCACATCTATAGTGACA | Antisense | 4984 |
| 209606_at | 195 | 25 | 1639 | ATAGGAGGGGTTAAGGTTCATGAGA | Antisense | 4985 |
| 209606_at | 790 | 823 | 1679 | GGTCTGAGATGGGTGCTGCAAAGAT | Antisense | 4986 |
| 209613_s_at | 312 | 531 | 1839 | GCAGATTTCTTGCTTCATATGACAA | Antisense | 4987 |
| 209613_s_at | 279 | 57 | 1857 | ATGACAAAGCCTCAATTACTAATTG | Antisense | 4988 |
| 209613_s_at | 372 | 235 | 1891 | AACTATTCCCAGAATCATGTTCAAA | Antisense | 4989 |
| 209613_s_at | 712 | 593 | 1938 | GAAAGTGCTTCATTGACTAAACAGT | Antisense | 4990 |
| 209613_s_at | 179 | 595 | 2100 | GAAATGGCACAAACACCTTAAGCAA | Antisense | 4991 |
| 209613_s_at | 560 | 153 | 2114 | ACCTTAAGCAATATATTTTCCTAGT | Antisense | 4992 |
| 209613_s_at | 332 | 579 | 2209 | GAACTTTTCTATTAACTACCATGTC | Antisense | 4993 |
| 209613_s_at | 119 | 277 | 2296 | AATGCTTTGTTAATGCTTTTCCCAC | Antisense | 4994 |
| 209613_s_at | 582 | 365 | 2311 | CTTTTCCCACTCTCATTTGTTAATG | Antisense | 4995 |
| 209613_s_at | 383 | 837 | 2354 | GGATTTGCATTTTGAGCTTTATCTC | Antisense | 4996 |
| 209613_s_at | 147 | 515 | 2369 | GCTTTATCTCTAAATGTGACATGCA | Antisense | 4997 |
| 209774_x_at | 524 | 115 | 477 | AGAGAGACACAGCTGCAGAGGCCAC | Antisense | 4998 |
| 209774_x_at | 464 | 157 | 500 | ACCTGGATTGCGCCTAATGTGTTTG | Antisense | 4999 |
| 209774_x_at | 864 | 933 | 627 | TGATTGAATCTACTTGCACACTCTC | Antisense | 5000 |
| 209774_x_at | 256 | 541 | 642 | GCACACTCTCCCATTATATTTATTG | Antisense | 5001 |
| 209774_x_at | 1059 | 239 | 681 | AACCCAAGTTAGTTCAATCCTGATT | Antisense | 5002 |
| 209774_x_at | 486 | 561 | 725 | GAAGGTTTGCAGATATTCTCTAGTC | Antisense | 5003 |
| 209774_x_at | 232 | 607 | 773 | GACATATCACATGTCAGCCACTGTG | Antisense | 5004 |
| 209774_x_at | 1021 | 71 | 822 | ATGGCCAGTAAGATCAATGTGACGG | Antisense | 5005 |
| 209774_x_at | 628 | 919 | 839 | TGTGACGGCAGGGAAATGTATGTGT | Antisense | 5006 |
| 209774_x_at | 281 | 337 | 920 | CAGTGTGTGGTCAACATTTCTCATG | Antisense | 5007 |
| 209774_x_at | 889 | 61 | 1023 | ATGTTAATTATGCAGTGTTTCCCTC | Antisense | 5008 |
| 209795_at | 451 | 1071 | 1143 | TAGTCTAATTGAATCCCTTAAACTC | Antisense | 5009 |
| 209795_at | 691 | 67 | 1273 | ATGGGATGATCGTGTATTTATTTTT | Antisense | 5010 |
| 209795_at | 665 | 1107 | 1294 | TTTTTTACTTCCTCAGCTGTAGACA | Antisense | 5011 |
| 209795_at | 1000 | 177 | 1300 | ACTTCCTCAGCTGTAGACAGGTCCT | Antisense | 5012 |
| 209795_at | 563 | 603 | 1315 | GACAGGTCCTTTTCGATGGTACATA | Antisense | 5013 |
| 209795_at | 188 | 907 | 1331 | TGGTACATATTTCTTTGCCTTTATA | Antisense | 5014 |
| 209795_at | 302 | 1083 | 1352 | TATAATCTTTTATACAGTGTCTTAC | Antisense | 5015 |
| 209795_at | 1015 | 773 | 1450 | GTGATGTGGCAAATCTCTATTAGGA | Antisense | 5016 |
| 209795_at | 970 | 21 | 1476 | ATATTCTGTAATCTTCAGACCTAGA | Antisense | 5017 |
| 209795_at | 859 | 83 | 1520 | AGGTTTGTGACTTTCCTAAATCAAT | Antisense | 5018 |
| 209795_at | 232 | 1059 | 1550 | TACGTGCAATACTTCAATACTTCAT | Antisense | 5019 |
| 209894_at | 1074 | 877 | 4425 | GGCATAGGAACAGTTTTCTCTTCAT | Antisense | 5020 |
| 209894_at | 1099 | 289 | 4484 | AATTCAGTATTTGTCATTATGCAGT | Antisense | 5021 |
| 209894_at | 37 | 31 | 4515 | ATACCTACATAAGTCTATTCCATTA | Antisense | 5022 |
| 209894_at | 605 | 1111 | 4545 | TTTTGCGCTTGGCATATTTATTCCT | Antisense | 5023 |
| 209894_at | 841 | 141 | 4666 | AGCTGGGTATACCATTATTTCAAGT | Antisense | 5024 |
| 209894_at | 967 | 581 | 4761 | GAACACATTTTCTATAGCCCTTAAT | Antisense | 5025 |
| 209894_at | 1019 | 1081 | 4773 | TATAGCCCTTAATTTAGTTGTGTTA | Antisense | 5026 |
| 209894_at | 156 | 819 | 4807 | GGTGATGTATCAACAGCTTTTTTTT | Antisense | 5027 |
| 209894_at | 378 | 725 | 4863 | GTATGAATTGAGCTTTTTGCCCACA | Antisense | 5028 |
| 209894_at | 641 | 1111 | 4877 | TTTTGCCCACAGATTCTTGATTTGT | Antisense | 5029 |
| 209894_at | 899 | 357 | 4892 | CTTGATTTGTAGTTGTTTGGCAGGA | Antisense | 5030 |
| 210260_s_at | 1031 | 693 | 1244 | GATTGAGTCATCGACATTCAGGATT | Antisense | 5031 |
| 210260_s_at | 234 | 1 | 1263 | AGGATTTAAGTCTGAGGTAGTCAAC | Antisense | 5032 |
| 210260_s_at | 946 | 219 | 1300 | AAAAATGGCTTATCTGAAATCAGTA | Antisense | 5033 |
| 210260_s_at | 544 | 55 | 1376 | ATGCTTCTGGAATTGAGTTCTCCTT | Antisense | 5034 |
| 210260_s_at | 142 | 647 | 1390 | GAGTTCTCCTTTTAAGTACCAATGA | Antisense | 5035 |
| 210260_s_at | 855 | 923 | 1434 | TGTAATGGTGTGTCATTGCCTTGAA | Antisense | 5036 |
| 210260_s_at | 897 | 779 | 1443 | GTGTCATTGCCTTGAAATGCTTGCT | Antisense | 5037 |
| 210260_s_at | 276 | 949 | 1455 | TGAAATGCTTGCTTAGGGCTTCTTT | Antisense | 5038 |
| 210260_s_at | 86 | 223 | 1546 | AAAAGTCTAGATTGGTCTTGATATT | Antisense | 5039 |
| 210260_s_at | 748 | 293 | 1607 | CAATCTTCATTCTACAGATGAACTC | Antisense | 5040 |
| 210260_s_at | 833 | 493 | 1701 | GCGTGCGTTTCAGTGTTTAAGAAGG | Antisense | 5041 |
| 210538_s_at | 297 | 593 | 2493 | GAAAGTGTCCTATTTGTAGGAGTAC | Antisense | 5042 |
| 210538_s_at | 59 | 847 | 2511 | GGAGTACAATCAAGGGTACAGTTCG | Antisense | 5043 |
| 210538_s_at | 551 | 735 | 2526 | GTACAGTTCGTACATTTCTTTCATG | Antisense | 5044 |
| 210538_s_at | 135 | 27 | 2741 | ATAGGCTTTTGTTCTTATGAACGAA | Antisense | 5045 |
| 210538_s_at | 909 | 253 | 2767 | AAGAGGTAGCACTACAAACACAATA | Antisense | 5046 |
| 210538_s_at | 97 | 287 | 2871 | AATATTTTGGCATTGTACTAATACC | Antisense | 5047 |
| 210538_s_at | 727 | 873 | 2932 | GGCAGCATACTGAGACCCTGCCTTT | Antisense | 5048 |
| 210538_s_at | 456 | 221 | 2978 | AAAACACCAGGGACACATTTCTCTG | Antisense | 5049 |
| 210538_s_at | 512 | 337 | 2985 | CAGGGACACATTTCTCTGTCTTTTT | Antisense | 5050 |
| 210538_s_at | 821 | 1091 | 3008 | TTTGATCAGTGTCCTATACATCGAA | Antisense | 5051 |
| 210538_s_at | 935 | 759 | 3018 | GTCCTATACATCGAAGGTGTGCATA | Antisense | 5052 |
| 210915_x_at | 1077 | 205 | 516 | AAAGGCCACACTGGTGTGCCTGGCC | Antisense | 5053 |
| 210915_x_at | 1116 | 945 | 651 | TGACTCCAGATACTGCCTGAGCAGC | Antisense | 5054 |
| 210915_x_at | 928 | 985 | 722 | TCCGCTGTCAAGTCCAGTTCTACGG | Antisense | 5055 |
| 210915_x_at | 495 | 419 | 807 | CGCCGAGGCCTGGGGTAGAGCAGAC | Antisense | 5056 |
| 210915_x_at | 185 | 1067 | 822 | TAGAGCAGACTGTGGCTTTACCTCG | Antisense | 5057 |
| 210915_x_at | 1098 | 461 | 875 | CCACCATCCTCTATGAGATCCTGCT | Antisense | 5058 |
| 210915_x_at | 577 | 109 | 890 | AGATCCTGCTAGGGAAGGCCACCAT | Antisense | 5059 |
| 210915_x_at | 857 | 269 | 904 | AAGGCCACCATGTATGCTGTGCTGG | Antisense | 5060 |
| 210915_x_at | 145 | 419 | 933 | CGCCCTTGTGTTGATGGCCATGGTC | Antisense | 5061 |
| 210915_x_at | 224 | 137 | 1001 | AGCTTCTAACCCGTCATGGTTTCAA | Antisense | 5062 |
| 210915_x_at | 864 | 293 | 1023 | CAATACACATTCTTCTTTTGCCAGC | Antisense | 5063 |
| 210972_x_at | 474 | 861 | 514 | GGAACAAGACTTCAGGTCACGCTCG | Antisense | 5064 |
| 210972_x_at | 393 | 1021 | 525 | TCAGGTCACGCTCGATATCCAGAAC | Antisense | 5065 |
| 210972_x_at | 111 | 949 | 591 | TGACAAGTCTGTCTGCCTATTCACC | Antisense | 5066 |
| 210972_x_at | 583 | 633 | 711 | GAGCAACAGTGCTGTGGCCTGGAGC | Antisense | 5067 |
| 210972_x_at | 60 | 525 | 751 | GCATGTGCAAACGCCTTCAACAACA | Antisense | 5068 |
| 210972_x_at | 317 | 987 | 800 | TCCCCAGCCCAGAAAGTTCCTGTGA | Antisense | 5069 |
| 210972_x_at | 461 | 701 | 815 | GTTCCTGTGATGTCAAGCTGGTCGA | Antisense | 5070 |
| 210972_x_at | 1124 | 335 | 884 | CAGTGATTGGGTTCCGAATCCTCCT | Antisense | 5071 |
| 210972_x_at | 920 | 263 | 914 | AAGTGGCCGGGTTTAATCTGCTCAT | Antisense | 5072 |
| 210972_x_at | 1017 | 497 | 948 | GCTGTGGTCCAGCTGAGATCTGCAA | Antisense | 5073 |
| 210972_x_at | 49 | 695 | 973 | GATTGTAAGACAGCCTGTGCTCCCT | Antisense | 5074 |
| 211603_s_at | 522 | 821 | 1524 | GGTGCTGCCCTGTGTACATATAAAT | Antisense | 5075 |
| 211603_s_at | 740 | 817 | 1555 | GGTGTTGGGGAAACCTTCATCTGAA | Antisense | 5076 |
| 211603_s_at | 193 | 121 | 1634 | AGACTCTGAGCTGCTCACCGGAGTC | Antisense | 5077 |
| 211603_s_at | 960 | 501 | 1643 | GCTGCTCACCGGAGTCATTGGGAAG | Antisense | 5078 |
| 211603_s_at | 23 | 371 | 1690 | CTAGAGTCTCAGAAACTCCCCTGGG | Antisense | 5079 |
| 211603_s_at | 491 | 843 | 1732 | GGAGGAATTCAGCTCAGCTTCTTCC | Antisense | 5080 |
| 211603_s_at | 664 | 195 | 1797 | ACAAGAGTTTGTTCTGTTCTGGGGG | Antisense | 5081 |
| 211603_s_at | 444 | 995 | 1814 | TCTGGGGGACAGAGAAGGCGCTTCC | Antisense | 5082 |
| 211603_s_at | 991 | 515 | 1833 | GCTTCCCAACTTCATACTGGCAGGA | Antisense | 5083 |
| 211603_s_at | 540 | 819 | 1859 | GGTGAGGAGGTTCACTGAGCTCCCC | Antisense | 5084 |
| 211603_s_at | 718 | 421 | 1890 | CCCACTGCGGGGAGACAGAAGCCTG | Antisense | 5085 |
| 211700_s_at | 28 | 461 | 2132 | CCAGCACGGGCTTCAGTAGTGGACC | Antisense | 5086 |
| 211700_s_at | 703 | 853 | 2152 | GGACCCAGTTCTATTGTTGGCTTCA | Antisense | 5087 |
| 211700_s_at | 182 | 517 | 2171 | GCTTCAGCGGTGGACCAAGCACTGG | Antisense | 5088 |
| 211700_s_at | 495 | 467 | 2185 | CCAAGCACTGGTGTTGGCTTCTGCA | Antisense | 5089 |
| 211700_s_at | 805 | 91 | 2209 | AGTGGACCAAGCACCAGTGGCTTCA | Antisense | 5090 |
| 211700_s_at | 183 | 517 | 2228 | GCTTCAGCGGTGGACCGAGCACAGG | Antisense | 5091 |
| 211700_s_at | 777 | 435 | 2242 | CCGAGCACAGGAGCTGGCTTCGGCG | Antisense | 5092 |
| 211700_s_at | 1099 | 357 | 2259 | CTTCGGCGGTGGACCAAACACTGGT | Antisense | 5093 |
| 211700_s_at | 995 | 157 | 2301 | ACCGAGCACCAGTGCTGGCTTTGGC | Antisense | 5094 |
| 211700_s_at | 396 | 967 | 2313 | TGCTGGCTTTGGCAGTGGAGCCGCC | Antisense | 5095 |
| 211700_s_at | 1091 | 789 | 2351 | GTGGCTTCTCGTATGGCTAGTGAGG | Antisense | 5096 |
| 211796_s_at | 480 | 475 | 459 | GCCATCAGAAGCAGAGATCTCCCAC | Antisense | 5097 |
| 211796_s_at | 1017 | 445 | 618 | CCTCAATGACTCCAGATACTGCCTG | Antisense | 5098 |
| 211796_s_at | 464 | 295 | 621 | CAATGACTCCAGATACTGCCTGAGC | Antisense | 5099 |
| 211796_s_at | 927 | 985 | 695 | TCCGCTGTCAAGTCCAGTTCTACGG | Antisense | 5100 |
| 211796_s_at | 588 | 401 | 699 | CTGTCAAGTCCAGTTCTACGGGCTC | Antisense | 5101 |
| 211796_s_at | 538 | 331 | 709 | CAGTTCTACGGGCTCTCGGAGAATG | Antisense | 5102 |
| 211796_s_at | 612 | 995 | 724 | TCGGAGAATGACGAGTGGACCCAGG | Antisense | 5103 |
| 211796_s_at | 969 | 253 | 730 | AATGACGAGTGGACCCAGGATAGGG | Antisense | 5104 |
| 211796_s_at | 396 | 437 | 782 | CCGAGGCCTGGGGTAGAGCAGACTG | Antisense | 5105 |
| 211796_s_at | 704 | 439 | 788 | CCTGGGGTAGAGCAGACTGTGGCTT | Antisense | 5106 |
| 211796_s_at | 1147 | 937 | 917 | TGATGGCCATGGTCAAGAGAAAGGA | Antisense | 5107 |
| 212587_s_at | 989 | 851 | 1559 | GGAGCCAATCCATGCAGATATTTTG | Antisense | 5108 |
| 212587_s_at | 733 | 895 | 1630 | TGGCTGAATTTCAGAGCATCCCGCG | Antisense | 5109 |
| 212587_s_at | 761 | 571 | 1680 | GAAGCTCGAAAGCCCTTTAACCAGA | Antisense | 5110 |
| 212587_s_at | 838 | 61 | 1717 | ATGTTGACATTCTTCCTTATGATTA | Antisense | 5111 |
| 212587_s_at | 302 | 689 | 1737 | GATTATAACCGTGTTGAACTCTCTG | Antisense | 5112 |
| 212587_s_at | 1002 | 807 | 1779 | GGGTCAAACTACATAAATGCCAGCT | Antisense | 5113 |
| 212587_s_at | 745 | 1053 | 1833 | TACATTGCTGCACAAGGTCCCAGGG | Antisense | 5114 |
| 212587_s_at | 885 | 469 | 1902 | GCCACAGTTATTGTCATGGTCACTC | Antisense | 5115 |
| 212587_s_at | 523 | 263 | 1950 | AAGTGTGCAGAATACTGGCCGTCAA | Antisense | 5116 |
| 212587_s_at | 755 | 889 | 1966 | GGCCGTCAATGGAAGAGGGCACTCG | Antisense | 5117 |
| 212587_s_at | 613 | 83 | 1981 | AGGGCACTCGGGCTTTTGGAGATGT | Antisense | 5118 |
| 212588_at | 847 | 983 | 4070 | TCCTCCTTGTTCTACTCATATATAT | Antisense | 5119 |
| 212588_at | 346 | 705 | 4078 | GTTCTACTCATATATATCTATCTTA | Antisense | 5120 |
| 212588_at | 445 | 1077 | 4146 | TATGTGTGTGTATGCTACTACAAAA | Antisense | 5121 |
| 212588_at | 820 | 7 | 4206 | ATTCCATATATTAGCATTTAGTCCA | Antisense | 5122 |
| 212588_at | 436 | 521 | 4219 | GCATTTAGTCCAATGTCTTTTTAAG | Antisense | 5123 |
| 212588_at | 93 | 773 | 4267 | GTGAGCTTATCATGCTGTCTTTACA | Antisense | 5124 |
| 212588_at | 306 | 497 | 4280 | GCTGTCTTTACATGGGGTTTTCAAT | Antisense | 5125 |
| 212588_at | 469 | 713 | 4296 | GTTTTCAATTTTGCATGCTCGATTA | Antisense | 5126 |
| 212588_at | 725 | 53 | 4310 | ATGCTCGATTATTCCCTGTACAATA | Antisense | 5127 |
| 212588_at | 421 | 1089 | 4344 | TATTGCTTGATACTTTTGACAACAA | Antisense | 5128 |
| 212588_at | 462 | 5 | 4543 | ATTGCATATGCATAGTTCCCATGTT | Antisense | 5129 |
| 212592_at | 641 | 723 | 859 | GTATCAAAATCTTCCAATTATCATG | Antisense | 5130 |
| 212592_at | 937 | 39 | 867 | ATCTTCCAATTATCATGCTCACCTG | Antisense | 5131 |
| 212592_at | 25 | 975 | 871 | TCCAATTATCATGCTCACCTGAAAG | Antisense | 5132 |
| 212592_at | 92 | 509 | 883 | GCTCACCTGAAAGAGGTATGCTCTC | Antisense | 5133 |
| 212592_at | 880 | 657 | 895 | GAGGTATGCTCTCTTAGGAATACAG | Antisense | 5134 |
| 212592_at | 700 | 1073 | 909 | TAGGAATACAGTTTCTAGCATTAAA | Antisense | 5135 |
| 212592_at | 187 | 1035 | 985 | TAATAAAATGTTCCTCGCATTCCCC | Antisense | 5136 |
| 212592_at | 374 | 103 | 1088 | AGTTTCACTGTGTAGAGAACATATA | Antisense | 5137 |
| 212592_at | 317 | 27 | 1122 | ATAGGTCAATTATATGTCTCCATTA | Antisense | 5138 |
| 212592_at | 790 | 217 | 1164 | AAACATGTTCTAGAACTAGTTACAA | Antisense | 5139 |
| 212592_at | 384 | 199 | 1248 | AAATATATTTGGACATAACAGACTT | Antisense | 5140 |
| 212654_at | 304 | 815 | 804 | GGTGGCCGAGAGTAAATGTGGGGAC | Antisense | 5141 |
| 212654_at | 339 | 737 | 815 | GTAAATGTGGGGACCTAGAGGAGGA | Antisense | 5142 |
| 212654_at | 180 | 903 | 872 | TGGAGGCCCAGGCGGACAAGTATTC | Antisense | 5143 |
| 212654_at | 145 | 341 | 880 | CAGGCGGACAAGTATTCCACCAAAG | Antisense | 5144 |
| 212654_at | 339 | 673 | 1024 | GATGAAGTCTATGCCCAGAAGATGA | Antisense | 5145 |
| 212654_at | 447 | 77 | 1067 | AGGAACTGGACAACGCACTCAATGA | Antisense | 5146 |
| 212654_at | 592 | 599 | 1075 | GACAACGCACTCAATGACATCACCT | Antisense | 5147 |
| 212654_at | 541 | 981 | 1149 | TCCTTTCCATTCTCTCTATGGGGAG | Antisense | 5148 |
| 212654_at | 13 | 661 | 1186 | GAGGAGCAGAAATTGCCAACATTGC | Antisense | 5149 |
| 212654_at | 183 | 1147 | 1198 | TTGCCAACATTGCACAGCCAGGCTG | Antisense | 5150 |
| 212654_at | 208 | 405 | 1220 | CTGGGAGCAGCCTAGGGAGAGCCCC | Antisense | 5151 |
| 212671_s_at | 689 | 149 | 439 | ACCAATGAGGTTCCTGAGGTCACAG | Antisense | 5152 |
| 212671_s_at | 593 | 59 | 443 | ATGAGGTTCCTGAGGTCACAGTGTT | Antisense | 5153 |
| 212671_s_at | 710 | 83 | 446 | AGGTTCCTGAGGTCACAGTGTTTTC | Antisense | 5154 |
| 212671_s_at | 604 | 983 | 450 | TCCTGAGGTCACAGTGTTTTCCAAG | Antisense | 5155 |
| 212671_s_at | 458 | 749 | 512 | GTCTTGTGGACAACATCTTTCCTCC | Antisense | 5156 |
| 212671_s_at | 615 | 383 | 604 | CTCTCCAAGAGTGATCATTCCTTCT | Antisense | 5157 |
| 212671_s_at | 807 | 467 | 608 | CCAAGAGTGATCATTCCTTCTTCAA | Antisense | 5158 |
| 212671_s_at | 232 | 113 | 611 | AGAGTGATCATTCCTTCTTCAAGAT | Antisense | 5159 |
| 212671_s_at | 793 | 93 | 613 | AGTGATCATTCCTTCTTCAAGATCA | Antisense | 5160 |
| 212671_s_at | 731 | 671 | 664 | GATGAGATTTATGACTGCAAGGTGG | Antisense | 5161 |
| 212671_s_at | 573 | 443 | 709 | CCTCTTCTGAAACACTGGGAGCCTG | Antisense | 5162 |
| 212813_at | 717 | 839 | 2297 | GGATGCTTAGCATGCAAGTTCCCTC | Antisense | 5163 |
| 212813_at | 955 | 389 | 2319 | CTCCATCATTGCCACCTTGGTAGAG | Antisense | 5164 |
| 212813_at | 300 | 907 | 2336 | TGGTAGAGAGGGATGGCTCCCCACC | Antisense | 5165 |
| 212813_at | 462 | 463 | 2356 | CCACCCTCAGCGTTGGGGATTCACG | Antisense | 5166 |
| 212813_at | 902 | 443 | 2387 | CCTCCTTCTTGGTTGTCATAGTGAT | Antisense | 5167 |
| 212813_at | 908 | 773 | 2407 | GTGATAGGGTAGCCTTATTGCCCCC | Antisense | 5168 |
| 212813_at | 33 | 1007 | 2453 | TCTACACTAGTGCCATGGGAACCAG | Antisense | 5169 |
| 212813_at | 55 | 771 | 2579 | GTGACTCAAGACTCGAGGCCGATAC | Antisense | 5170 |
| 212813_at | 978 | 483 | 2596 | GCCGATACGAGGCTGTGATTCTGCC | Antisense | 5171 |
| 212813_at | 188 | 693 | 2612 | GATTCTGCCTTTGGATGGATGTTGC | Antisense | 5172 |
| 212813_at | 236 | 1053 | 2650 | TACAGACTTGTACTAACACACCGTA | Antisense | 5173 |
| 213193_x_at | 1117 | 945 | 664 | TGACTCCAGATACTGCCTGAGCAGC | Antisense | 5174 |
| 213193_x_at | 926 | 985 | 735 | TCCGCTGTCAAGTCCAGTTCTACGG | Antisense | 5175 |
| 213193_x_at | 186 | 1067 | 835 | TAGAGCAGACTGTGGCTTTACCTCG | Antisense | 5176 |
| 213193_x_at | 1097 | 461 | 888 | CCACCATCCTCTATGAGATCCTGCT | Antisense | 5177 |
| 213193_x_at | 254 | 643 | 902 | GAGATCCTGCTAGGGAAGGCCACCC | Antisense | 5178 |
| 213193_x_at | 74 | 319 | 922 | CACCCTGTATGCTGTGCTGGTCAGC | Antisense | 5179 |
| 213193_x_at | 863 | 293 | 1036 | CAATACACATTCTTCTTTTGCCAGC | Antisense | 5180 |
| 213193_x_at | 1099 | 471 | 1055 | GCCAGCGCTTCTGAAGAGCTGCTCT | Antisense | 5181 |
| 213193_x_at | 522 | 155 | 1081 | ACCTCTCTGCATCCCAATAGATATC | Antisense | 5182 |
| 213193_x_at | 717 | 283 | 1096 | AATAGATATCCCCCTATGTGCATGC | Antisense | 5183 |
| 213193_x_at | 782 | 811 | 1158 | GGGGGACCTTAGCATGCCTAAGTGA | Antisense | 5184 |
| 213539_at | 163 | 799 | 265 | GGGAACACTGCTCTCAGACATTACA | Antisense | 5185 |
| 213539_at | 711 | 613 | 291 | GACTGGACCTGGGAAAACGCATCCT | Antisense | 5186 |
| 213539_at | 701 | 417 | 308 | CGCATCCTGGACCCACGAGGAATAT | Antisense | 5187 |
| 213539_at | 383 | 553 | 368 | GAATCTACCGTGCAAGTTCATTATC | Antisense | 5188 |
| 213539_at | 835 | 781 | 408 | GTGTGGAGCTGGATCCAGCCACCGT | Antisense | 5189 |
| 213539_at | 610 | 119 | 507 | AGACTGGAAGGCTGTCTGGGGCTGC | Antisense | 5190 |
| 213539_at | 108 | 971 | 529 | TGCCGACACACAAGCTCTGTTGAGG | Antisense | 5191 |
| 213539_at | 964 | 867 | 552 | GGAATGACCAGGTCTATCAGCCCCT | Antisense | 5192 |
| 213539_at | 659 | 145 | 570 | AGCCCCTCCGAGATCGAGATGATGC | Antisense | 5193 |
| 213539_at | 1113 | 961 | 592 | TGCTCAGTACAGCCACCTTGGAGGA | Antisense | 5194 |
| 213539_at | 892 | 569 | 659 | GAAGCAGCCATTACCAACTGTACCT | Antisense | 5195 |
| 213790_at | 850 | 717 | 1130 | GTTTGAGTTTTGTAGGGCCATCACC | Antisense | 5196 |
| 213790_at | 16 | 793 | 1144 | GGGCCATCACCTGGAAAGTCAATGT | Antisense | 5197 |
| 213790_at | 719 | 373 | 1194 | CTACTTTTCTTCCTACAGCTTATTA | Antisense | 5198 |
| 213790_at | 619 | 727 | 1224 | GTAGGTTCTATGACCTCACTTCATG | Antisense | 5199 |
| 213790_at | 83 | 313 | 1240 | CACTTCATGGGTTCCAGGCAATTCC | Antisense | 5200 |
| 213790_at | 478 | 547 | 1257 | GCAATTCCGCTGAAAGGTTTGTCTC | Antisense | 5201 |
| 213790_at | 709 | 573 | 1345 | GAAGCCTAACATTCTCCATTGTGGA | Antisense | 5202 |
| 213790_at | 854 | 1105 | 1404 | TTTTTCCAGCTTACACATGTGGGAT | Antisense | 5203 |
| 213790_at | 979 | 847 | 1468 | GGAGAGTCAATACTCCTGACGAGTC | Antisense | 5204 |
| 213790_at | 86 | 813 | 1500 | GGGGCATTTTTATGCCTTCTTAACT | Antisense | 5205 |
| 213790_at | 942 | 883 | 1540 | GGCTGAACTATAGGCCATTGTTCCC | Antisense | 5206 |
| 214470_at | 865 | 831 | 172 | GGTTCACCTTGGCATCAATTTGCCC | Antisense | 5207 |
| 214470_at | 556 | 1095 | 190 | TTTGCCCTGAAACTTAGCTGTGCTG | Antisense | 5208 |
| 214470_at | 660 | 141 | 205 | AGCTGTGCTGGGATTATTCTCCTTG | Antisense | 5209 |
| 214470_at | 519 | 9 | 220 | ATTCTCCTTGTCTTGGTTGTTACTG | Antisense | 5210 |
| 214470_at | 1064 | 805 | 244 | GGGTTGAGTGTTTCAGTGACATCCT | Antisense | 5211 |
| 214470_at | 1050 | 437 | 340 | CCGGGTCTCTTAAACTGCCCAATAT | Antisense | 5212 |
| 214470_at | 1032 | 929 | 392 | TGTTATTTTCTCACACTGTCAACCC | Antisense | 5213 |
| 214470_at | 965 | 229 | 424 | AACAGTCTAGCTGATTGTTCCACCA | Antisense | 5214 |
| 214470_at | 59 | 1153 | 438 | TTGTTCCACCAAAGAATCCAGCCTG | Antisense | 5215 |
| 214470_at | 186 | 977 | 454 | TCCAGCCTGCTGCTTATTCGAGATA | Antisense | 5216 |
| 214470_at | 1161 | 219 | 632 | AAAACAGCTGTATTTCCATCTCACA | Antisense | 5217 |
| 215313_x_at | 921 | 941 | 902 | TGAGATGGGAGCTGTCTTCCCAGCC | Antisense | 5218 |
| 215313_x_at | 649 | 347 | 936 | CATCGTGGGCATCATTGCTGGCCTG | Antisense | 5219 |
| 215313_x_at | 825 | 935 | 977 | TGATCACTGGAGCTGTGGTCGCTGC | Antisense | 5220 |
| 215313_x_at | 305 | 757 | 994 | GTCGCTGCCGTGATGTGGAGGAGGA | Antisense | 5221 |
| 215313_x_at | 1053 | 883 | 1071 | GGCTGCAAGCAGTGACAGTGCCCAG | Antisense | 5222 |
| 215313_x_at | 582 | 121 | 1131 | AGACAGCTGCCTTGTGTGGGACTGA | Antisense | 5223 |
| 215313_x_at | 475 | 569 | 1193 | GAAGAACCCTGACTTTGTTTCTGCA | Antisense | 5224 |
| 215313_x_at | 837 | 959 | 1226 | TGCATGTGTCTGTGTTCGTGTAGGC | Antisense | 5225 |
| 215313_x_at | 1069 | 293 | 1331 | CAATCATCTTTCCTGTTCCAGAGAG | Antisense | 5226 |
| 215313_x_at | 124 | 349 | 1374 | CATCTCTGTCTCAACTTCATGGTGC | Antisense | 5227 |
| 215313_x_at | 634 | 907 | 1393 | TGGTGCACTGAGCTGTAACTTCTTC | Antisense | 5228 |
| 216438_s_at | 1086 | 185 | 150 | ACAGAGACGCAAGAGAAAAATCCAC | Antisense | 5229 |
| 216438_s_at | 546 | 277 | 168 | AATCCACTGCCTTCCAAAGAAACGA | Antisense | 5230 |
| 216438_s_at | 138 | 315 | 172 | CACTGCCTTCCAAAGAAACGATTGA | Antisense | 5231 |
| 216438_s_at | 670 | 849 | 200 | GGAGAAGCAAGCAGGCGAATCGTAA | Antisense | 5232 |
| 216438_s_at | 49 | 135 | 209 | AGCAGGCGAATCGTAATGAGGCGTG | Antisense | 5233 |
| 216438_s_at | 556 | 957 | 245 | TGCACTGTACATTCCACAAGCATTG | Antisense | 5234 |
| 216438_s_at | 569 | 167 | 248 | ACTGTACATTCCACAAGCATTGCCT | Antisense | 5235 |
| 216438_s_at | 9 | 109 | 308 | AGATGCAAAGAGGTTGGATCAAGTT | Antisense | 5236 |
| 216438_s_at | 178 | 259 | 328 | AAGTTTAAATGACTGTGCTGCCCCT | Antisense | 5237 |
| 216438_s_at | 744 | 591 | 426 | GAAAGAACTTGCATGTTGGTGAAGG | Antisense | 5238 |
| 216438_s_at | 432 | 499 | 516 | GCTGTAATGCAGTTTAATCAGAGTG | Antisense | 5239 |
| 216920_s_at | 416 | 313 | 881 | CACTACTGCTGCAGCTCACAAACAC | Antisense | 5240 |
| 216920_s_at | 585 | 509 | 894 | GCTCACAAACACCTCTGCATATTAC | Antisense | 5241 |
| 216920_s_at | 504 | 1001 | 907 | TCTGCATATTACATGTACCTCCTCC | Antisense | 5242 |
| 216920_s_at | 513 | 783 | 944 | GTGTGGTCTATTTTGCCATCATCAC | Antisense | 5243 |
| 216920_s_at | 940 | 891 | 1115 | GGCCATTTCAGTTCTCATGTGTGTA | Antisense | 5244 |
| 216920_s_at | 978 | 829 | 1161 | GGTTTTCAAACCAGTGGGCACACAG | Antisense | 5245 |
| 216920_s_at | 584 | 989 | 1323 | TCCCTCTCTTAGTGTTCTTTAATCA | Antisense | 5246 |
| 216920_s_at | 126 | 35 | 1349 | ATAACTGCCTGGAAGCCTTTCATTT | Antisense | 5247 |
| 216920_s_at | 170 | 897 | 1358 | TGGAAGCCTTTCATTTTACACGCCC | Antisense | 5248 |
| 216920_s_at | 1055 | 417 | 1378 | CGCCCTGAAGCAGTCTTCTTTGCTA | Antisense | 5249 |
| 216920_s_at | 1041 | 1115 | 1409 | TTATGTGGTGTGTTTTTCCGTAATA | Antisense | 5250 |
| 217767_at | 461 | 823 | 4506 | GGTCTACGCCTATTACAACCTGGAG | Antisense | 5251 |
| 217767_at | 1003 | 247 | 4582 | AAGCTCTGCCGTGATGAACTGTGCC | Antisense | 5252 |
| 217767_at | 579 | 577 | 4663 | GAACGGCTGGACAAGGCCTGTGAGC | Antisense | 5253 |
| 217767_at | 98 | 833 | 4725 | GGTTCAGCTGTCCAATGACTTTGAC | Antisense | 5254 |
| 217767_at | 994 | 531 | 4770 | GCAGACCATCAAGTCAGGCTCGGAT | Antisense | 5255 |
| 217767_at | 382 | 1017 | 4823 | TCATCAGCCCCATCAAGTGCAGAGA | Antisense | 5256 |
| 217767_at | 347 | 227 | 4871 | AACACTACCTCATGTGGGGTCTCTC | Antisense | 5257 |
| 217767_at | 848 | 751 | 4889 | GTCTCTCCTCCGATTTCTGGGGAGA | Antisense | 5258 |
| 217767_at | 1105 | 787 | 4954 | GTGGAGCACTGGCCTGAGGAGGACG | Antisense | 5259 |
| 217767_at | 105 | 123 | 5000 | AGAAACAATGCCAGGACCTCGGCGC | Antisense | 5260 |
| 217767_at | 589 | 419 | 5022 | CGCCTTCACCGAGAGCATGGTTGTC | Antisense | 5261 |
| 218499_at | 1142 | 271 | 2607 | AATGTTTAATTGTTTGGATCTGCAC | Antisense | 5262 |
| 218499_at | 539 | 839 | 2622 | GGATCTGCACAGTTTGGTTTTTGCA | Antisense | 5263 |
| 218499_at | 302 | 159 | 2716 | ACAGTTTTTAGTCAAAGTGGCCATT | Antisense | 5264 |
| 218499_at | 1129 | 289 | 2808 | AATTCTTTTTATTGGTGCCTATATT | Antisense | 5265 |
| 218499_at | 850 | 1129 | 2873 | TTCAGTTAAATTTTTCACCTGCTGT | Antisense | 5266 |
| 218499_at | 832 | 847 | 2942 | GGAGATAATTTTGCAACTCATGTTA | Antisense | 5267 |
| 218499_at | 444 | 175 | 3028 | ACTTTACAACACAATTGCATCCCAA | Antisense | 5268 |
| 218499_at | 24 | 1089 | 3070 | TATTCATTATAGCTATTCGTCCTGT | Antisense | 5269 |
| 218499_at | 499 | 1089 | 3083 | TATTCGTCCTGTAATCTGTTTCTAG | Antisense | 5270 |
| 218499_at | 957 | 715 | 3100 | GTTTCTAGGTGAAGCATACTCCAGT | Antisense | 5271 |
| 218499_at | 528 | 519 | 3113 | GCATACTCCAGTGTTTTAGGGGTTT | Antisense | 5272 |
| 218805_at | 459 | 1049 | 1237 | TAAATCTCTGGACCCTGGAGCACTT | Antisense | 5273 |
| 218805_at | 2 | 903 | 1252 | TGGAGCACTTCTAATGTATCACCCC | Antisense | 5274 |
| 218805_at | 1057 | 721 | 1267 | GTATCACCCCATGGAGTCATTGTTC | Antisense | 5275 |
| 218805_at | 1042 | 611 | 1309 | GACTCAGATCCTCGTGGTCTATGGA | Antisense | 5276 |
| 218805_at | 1072 | 987 | 1366 | TCCCCTTCTTCCTGATAGACTTGGA | Antisense | 5277 |
| 218805_at | 377 | 971 | 1414 | TGCCTGCCTGCTGTAAACACTATTC | Antisense | 5278 |
| 218805_at | 274 | 997 | 1442 | TCTGTCTGCCAACAACTGCTTCAGG | Antisense | 5279 |
| 218805_at | 121 | 451 | 1540 | CCTTAGATAGAACCTGTCTTCCTCC | Antisense | 5280 |
| 218805_at | 482 | 387 | 1561 | CTCCCTGGCATTGTGGGGTCTGGGC | Antisense | 5281 |
| 218805_at | 136 | 369 | 1608 | CTTTGTGCTGCCAACCTGAGATTGA | Antisense | 5282 |
| 218805_at | 814 | 1079 | 1705 | TATCTCCGCATTTCCAGTTGTATTA | Antisense | 5283 |
| 218807_at | 1032 | 969 | 4366 | TGCCGTCAGCCGAACTTTGTTATGG | Antisense | 5284 |
| 218807_at | 727 | 903 | 4388 | TGGAGGGAGCAGCCTCACACAAGCA | Antisense | 5285 |
| 218807_at | 584 | 587 | 4413 | GAAACACTCCTGTGGATGGTATTGT | Antisense | 5286 |
| 218807_at | 1142 | 1067 | 4456 | TAGTCAATAGACCCTCTCCTTATAA | Antisense | 5287 |
| 218807_at | 255 | 531 | 4529 | GCAGAGGATATTGGAGCCCCTTTTT | Antisense | 5288 |
| 218807_at | 886 | 53 | 4543 | AGCCCCTTTTTGTGACATTACCAAT | Antisense | 5289 |
| 218807_at | 639 | 1063 | 4561 | TACCAATTACATCTTTGTCCACGTT | Antisense | 5290 |
| 218807_at | 420 | 921 | 4576 | TGTCCACGTTTAATACTTTGTTTTG | Antisense | 5291 |
| 218807_at | 77 | 63 | 4656 | ATGTTTTCCATATACTTTGTCTTGC | Antisense | 5292 |
| 218807_at | 752 | 1055 | 4668 | TACTTTGTCTTGCCTGTATGCAGCC | Antisense | 5293 |
| 218807_at | 355 | 723 | 4683 | GTATGCAGCCCTTGTGTAATATGGT | Antisense | 5294 |
| 222838_at | 228 | 229 | 2352 | AACACCTGTGCTAGGTCAGTCTGGC | Antisense | 5295 |
| 222838_at | 971 | 509 | 2361 | GCTAGGTCAGTCTGGCACGTAAGAT | Antisense | 5296 |
| 222838_at | 805 | 755 | 2370 | GTCTGGCACGTAAGATGAACATCCC | Antisense | 5297 |
| 222838_at | 1054 | 1039 | 2380 | TAAGATGAACATCCCTACCAACACA | Antisense | 5298 |
| 222838_at | 728 | 629 | 2405 | GAGCTCACCATCTCTTATACTTAAG | Antisense | 5299 |
| 222838_at | 821 | 61 | 2473 | ATGTTCCCTGACACATATCTTGAAT | Antisense | 5300 |
| 222838_at | 479 | 179 | 2485 | ACATATCTTGAATGGAGACCTCCCT | Antisense | 5301 |
| 222838_at | 379 | 119 | 2500 | AGACCTCCCTACCAAGTGATGAAAG | Antisense | 5302 |
| 222838_at | 520 | 805 | 2565 | GGGATTGAGGATTATCTTCTCTCAG | Antisense | 5303 |
| 222838_at | 382 | 1013 | 2579 | TCTTCTCTCAGAAAGGCATTGTGAA | Antisense | 5304 |
| 222838_at | 797 | 447 | 2626 | CCTACTGCAAAACCCTATTGTAGTA | Antisense | 5305 |
| 222943_at | 191 | 561 | 1738 | GAAGAGAAAACCACTAACCTTGATT | Antisense | 5306 |
| 222943_at | 1056 | 243 | 1855 | AACCATGATTTGCAACTGTAACAGG | Antisense | 5307 |
| 222943_at | 511 | 623 | 1881 | GACCATTTATTATAAGCGTACCTGT | Antisense | 5308 |
| 222943_at | 16 | 247 | 1894 | AAGCGTACCTGTTTGTGAACTTAAT | Antisense | 5309 |
| 222943_at | 812 | 499 | 1935 | GCTGTTTTTGCTTAGGTGATCCACT | Antisense | 5310 |
| 222943_at | 838 | 51 | 1953 | ATCCACTGCCATGTGATCCATAATT | Antisense | 5311 |
| 222943_at | 119 | 775 | 1965 | GTGATCCATAATTTTTCTACATAAA | Antisense | 5312 |
| 222943_at | 535 | 1007 | 2055 | TCTACTTGTTAATATATTCGGCCCA | Antisense | 5313 |
| 222943_at | 304 | 9 | 2070 | ATTCGGCCCATATTTTGTGTGTTTG | Antisense | 5314 |
| 222943_at | 1105 | 781 | 2086 | GTGTGTTTGGACAAGTACATCTCCC | Antisense | 5315 |
| 222943_at | 813 | 1003 | 2105 | TCTCCCTTTTGCCTAATGAACTTTT | Antisense | 5316 |
| 223403_s_at | 938 | 353 | 1845 | CATTACCAGGTTACTCTTGAGATTT | Antisense | 5317 |
| 223403_s_at | 582 | 709 | 1879 | GTTAGAACTCTCAACCAAGACCTGA | Antisense | 5318 |
| 223403_s_at | 445 | 725 | 1911 | GTATGCAAGGTTTCTGAATCTCTCT | Antisense | 5319 |
| 223403_s_at | 603 | 705 | 1974 | GTTCAAAAAGTTCATGTCTTCTCAA | Antisense | 5320 |
| 223403_s_at | 959 | 865 | 2019 | GGAAGAGCATACGGTGACAAGTCTC | Antisense | 5321 |
| 223403_s_at | 338 | 191 | 2097 | ACACCTGTTAATCCATCTTGAGCAG | Antisense | 5322 |
| 223403_s_at | 703 | 41 | 2111 | ATCTTGAGCAGGACAGTACTATACA | Antisense | 5323 |
| 223403_s_at | 253 | 1087 | 2165 | TATTTTCTTATAGCCACGTTGAAGT | Antisense | 5324 |
| 223403_s_at | 234 | 1053 | 2203 | TACAGTGTTTTTTACCAGCTTTATA | Antisense | 5325 |
| 223403_s_at | 300 | 667 | 2264 | GATGGGTCTGACTATATGCACACAC | Antisense | 5326 |
| 223403_s_at | 1064 | 55 | 2279 | ATGCACACACCTTTGATACCATCAC | Antisense | 5327 |
| 223593_at | 41 | 875 | 1467 | GGCAGCTGCAGACAAGTGGTTAACT | Antisense | 5328 |
| 223593_at | 336 | 595 | 1477 | GACAAGTGGTTAACTGGTTTGGCAG | Antisense | 5329 |
| 223593_at | 920 | 1159 | 1495 | TTGGCAGAATGGCATGTTCCTGCTG | Antisense | 5330 |
| 223593_at | 255 | 667 | 1593 | GATGGGGGTATTAATGCTCCCTGGA | Antisense | 5331 |
| 223593_at | 9 | 505 | 1608 | GCTCCCTGGAAATGCTTTCTACGTC | Antisense | 5332 |
| 223593_at | 1103 | 371 | 1626 | CTACGTCGATAGCTCAGCTCCTAGC | Antisense | 5333 |
| 223593_at | 458 | 981 | 1644 | TCCTAGCCCTTACTTGAGAGCATCC | Antisense | 5334 |
| 223593_at | 768 | 667 | 1696 | GATGTGGCCTTCCAGGTATTAGCAC | Antisense | 5335 |
| 223593_at | 444 | 891 | 1701 | GGCCTTCCAGGTATTAGCACAACTT | Antisense | 5336 |
| 223593_at | 635 | 171 | 1884 | ACTCTGGCCTGCATGGCAGAACAAG | Antisense | 5337 |
| 223593_at | 445 | 201 | 1934 | AAATCAAACTAATCATGCTGCTCAT | Antisense | 5338 |
| 224896_s_at | 643 | 1001 | 3108 | TTCCCCAAAACTCAACTCCTATGGC | Antisense | 5339 |
| 224896_s_at | 581 | 869 | 3130 | GGCAATTATGAACTCCATTTTACCA | Antisense | 5340 |
| 224896_s_at | 927 | 251 | 3154 | AAGAACATTTAAGTGCCTCAGCATC | Antisense | 5341 |
| 224896_s_at | 670 | 261 | 3164 | AAGTGCCTCAGCATCTGTATGATAT | Antisense | 5342 |
| 224896_s_at | 13 | 27 | 3207 | ATAGGTACCAGCTGACATGATGTGT | Antisense | 5343 |
| 224896_s_at | 22 | 669 | 3225 | GATGTGTCACTAGCTCTGTGGGATG | Antisense | 5344 |
| 224896_s_at | 913 | 183 | 3260 | ACATGGAACACCTGGGAGTGCTGGA | Antisense | 5345 |
| 224896_s_at | 1162 | 761 | 3277 | GTGCTGGAAATGTACTGGGATCGAA | Antisense | 5346 |
| 224896_s_at | 940 | 181 | 3397 | ACATGGGCACCGGAGTAGGTCCCGT | Antisense | 5347 |
| 224896_s_at | 451 | 335 | 3450 | CAGTGACGTACATGGCTCTGGTTCT | Antisense | 5348 |
| 224896_s_at | 577 | 833 | 3469 | GGTTCTGGACACAAAATCTGTACTG | Antisense | 5349 |
| 224908_s_at | 277 | 149 | 2301 | ACCAAGCCTGGCTTTATGTATTTAT | Antisense | 5350 |
| 224908_s_at | 343 | 187 | 2387 | ACAGAAGGTTATGCCTGGCTCCCAG | Antisense | 5351 |
| 224908_s_at | 348 | 909 | 2454 | TGGGGGAGGAGTCATGGTTTATTTG | Antisense | 5352 |
| 224908_s_at | 558 | 969 | 2549 | TGCCTTAGTTTCTTAGCCCATGAAA | Antisense | 5353 |
| 224908_s_at | 500 | 619 | 2587 | GACCCAGGGACTACCTCAAGGGCTT | Antisense | 5354 |
| 224908_s_at | 1051 | 865 | 2634 | GGAAGATGCAAGAGCCTTTAGTACC | Antisense | 5355 |
| 224908_s_at | 192 | 117 | 2644 | AGAGCCTTTAGTACCAAGGTTCTCA | Antisense | 5356 |
| 224908_s_at | 658 | 425 | 2738 | CCCGGACAGTTAAATCAGAACCTCA | Antisense | 5357 |
| 224908_s_at | 811 | 327 | 2753 | CAGAACCTCAGACAGCAATATGCCT | Antisense | 5358 |
| 224908_s_at | 319 | 21 | 2770 | ATATGCCTTGAGATGCCTTGAACCA | Antisense | 5359 |
| 224908_s_at | 464 | 643 | 2779 | GAGATGCCTTGAACCATGCTTGAGA | Antisense | 5360 |
| 225502_at | 1153 | 571 | 5134 | GAAGCCTTACTACAATTCCAAAAAT | Antisense | 5361 |
| 225502_at | 895 | 39 | 5157 | ATCATCATGGTTGGAAATTTGGGAG | Antisense | 5362 |
| 225502_at | 522 | 1085 | 5187 | TATTTGTGAACTTGTTACCCTTTTG | Antisense | 5363 |
| 225502_at | 162 | 741 | 5212 | GTAATGGTGGACTAATTGCTGTATA | Antisense | 5364 |
| 225502_at | 885 | 1051 | 5319 | TACACGGTACTTGGAGCAGTCAGCC | Antisense | 5365 |
| 225502_at | 659 | 1029 | 5349 | TCACAGATACTGCTTTCACTTAAAT | Antisense | 5366 |
| 225502_at | 87 | 291 | 5380 | AATTCTCCGATAATGCTTTGCTTTT | Antisense | 5367 |
| 225502_at | 1017 | 743 | 5413 | GTCACTCTTGTGTACTATCTATTTT | Antisense | 5368 |
| 225502_at | 324 | 1159 | 5420 | TTGTGTACTATCTATTTTTCTCCTC | Antisense | 5369 |
| 225502_at | 283 | 251 | 5469 | AAGCAATAATATCTCTGTTTTCATT | Antisense | 5370 |
| 225502_at | 218 | 45 | 5479 | ATCTCTGTTTTCATTTCAGAACATT | Antisense | 5371 |
| 225882_at | 1108 | 535 | 2178 | GCACCTTACATATTTGATGCTCAGG | Antisense | 5372 |
| 225882_at | 944 | 461 | 2283 | CCACCCTTACATCTCCATAGTTGGT | Antisense | 5373 |
| 225882_at | 1067 | 695 | 2302 | GTTGGTACAGTTAGCTTGTAGCAGC | Antisense | 5374 |
| 225882_at | 561 | 51 | 2335 | ATGCCTGGAGATCACTGTCTGTTGG | Antisense | 5375 |
| 225882_at | 429 | 401 | 2349 | CTGTCTGTTGGTCTGATCTCAGTAT | Antisense | 5376 |
| 225882_at | 844 | 333 | 2368 | CAGTATCATACTGAGACACCTCCCC | Antisense | 5377 |
| 225882_at | 909 | 441 | 2391 | CCTGAGCCTTACCTACTTAAATTGG | Antisense | 5378 |
| 225882_at | 899 | 661 | 2453 | GAGGCAAGTTGCAAGGGAGCCAGAG | Antisense | 5379 |
| 225882_at | 841 | 285 | 2534 | AATTTCTTTGGCACATTGACTTACT | Antisense | 5380 |
| 225882_at | 169 | 873 | 2543 | GGCACATTGACTTACTGATATCTTT | Antisense | 5381 |
| 225882_at | 408 | 1001 | 2614 | TATTCCCCGGCGCAATAAAAATGCC | Antisense | 5382 |
| 226218_at | 938 | 375 | 979 | CTAATTGGTTCTGCCCAATCTCCTT | Antisense | 5383 |
| 226218_at | 739 | 943 | 1126 | TGACTGGGTCTAGGGCACCCAGGCT | Antisense | 5384 |
| 226218_at | 810 | 691 | 1151 | GATTCAGCTGATTTCCTACCAGCCT | Antisense | 5385 |
| 226218_at | 730 | 1075 | 1229 | TATGGGCTGTTCAGAGGTGCACACC | Antisense | 5386 |
| 226218_at | 1063 | 249 | 1283 | AAGAGACTTGGTACGGGCCAGGAAG | Antisense | 5387 |
| 226218_at | 819 | 551 | 1307 | GAATATGTGGCAGAGCTCCTGGAAA | Antisense | 5388 |
| 226218_at | 949 | 1073 | 1342 | TAGGTGGCATTTTTGTCAGCTCTGT | Antisense | 5389 |
| 226218_at | 721 | 745 | 1356 | GTCAGCTCTGTGGTTTATTGTTGGG | Antisense | 5390 |
| 226218_at | 938 | 199 | 1392 | AAATATCCATTGTTCACTACAGTGA | Antisense | 5391 |
| 226218_at | 984 | 157 | 1431 | ACCGTGTACTATCCACATGCATTAC | Antisense | 5392 |
| 226218_at | 64 | 13 | 1451 | ATTACAAACATTTCGCAGAGCTGCT | Antisense | 5393 |
| 226219_at | 896 | 751 | 1088 | GTCTCCGACACAAAGCACTTATCTC | Antisense | 5394 |
| 226219_at | 539 | 539 | 1102 | GCACTTATCTCTTAGGAGATTCCCA | Antisense | 5395 |
| 226219_at | 56 | 109 | 1139 | AGATCTTGTTCCCAGGGAGTGGGTC | Antisense | 5396 |
| 226219_at | 447 | 983 | 1241 | TCCTCTGAGCTGTGAATCTCTCTTC | Antisense | 5397 |
| 226219_at | 93 | 217 | 1323 | AAACACTTCTAGAGTTGCCCATTCC | Antisense | 5398 |
| 226219_at | 933 | 487 | 1339 | GCCCATTCCTGTTATGTTCTTGGAC | Antisense | 5399 |
| 226219_at | 527 | 705 | 1354 | GTTCTTGGACCCTAAGATACCTCCT | Antisense | 5400 |
| 226219_at | 599 | 1151 | 1424 | TTGAAGCCCTCAATATTTGTTGGAG | Antisense | 5401 |
| 226219_at | 753 | 767 | 1496 | GTGCAAGAGAGGTCCTGTACAGATC | Antisense | 5402 |
| 226219_at | 1151 | 1001 | 1530 | TCTCCTTTCTCCTTTGGAATAACTT | Antisense | 5403 |
| 226219_at | 537 | 465 | 1623 | CCACAGGTCCCACTCTAGTGAAGGT | Antisense | 5404 |
| 227265_at | 1000 | 181 | 1401 | ACATCTGCTAGAACCTTTTGCCTTA | Antisense | 5405 |
| 227265_at | 885 | 449 | 1414 | CCTTTTGCCTTAACTATTCACCAAT | Antisense | 5406 |
| 227265_at | 319 | 13 | 1480 | ATTAGAATCTTGTCTTGGTTCCATT | Antisense | 5407 |
| 227265_at | 779 | 665 | 1507 | GATGGCTAATATTTGTTATCTTAAT | Antisense | 5408 |
| 227265_at | 325 | 19 | 1539 | ATTTCTGAGGTCATGATTACTTGAA | Antisense | 5409 |
| 227265_at | 864 | 1 | 1565 | ATATTGACTAAAACTGGGTCCTTAG | Antisense | 5410 |
| 227265_at | 290 | 805 | 1580 | GGGTCCTTAGAAATTCCAGGTGGAG | Antisense | 5411 |
| 227265_at | 128 | 789 | 1599 | GTGGAGCTGATTTACCTATGACTGA | Antisense | 5412 |
| 227265_at | 512 | 289 | 1671 | AATTAATGACACATCTGTTCAATAA | Antisense | 5413 |
| 227265_at | 1066 | 739 | 1802 | GTAATGTCATCACAATAAGCTCTTG | Antisense | 5414 |
| 227265_at | 778 | 277 | 1852 | AATTAGGTTTATGTTTCATGTCTTT | Antisense | 5415 |
| 227346_at | 745 | 961 | 407 | TGCTTTTGGTGCGATGGCACTCACT | Antisense | 5416 |
| 227346_at | 845 | 547 | 460 | GCAATATTGTTTCCAATACTTTCTA | Antisense | 5417 |
| 227346_at | 374 | 783 | 507 | GTGTGGTGATTGTTCAGGTCGAATC | Antisense | 5418 |
| 227346_at | 83 | 85 | 522 | AGGTCGAATCTGTTGTATCCAGTAC | Antisense | 5419 |
| 227346_at | 359 | 51 | 538 | ATCCAGTACAGCTTTAGGTCTTCAG | Antisense | 5420 |
| 227346_at | 120 | 393 | 563 | CTGCCCTTCTGGCGAGTACATGCAC | Antisense | 5421 |
| 227346_at | 211 | 959 | 606 | TGCAGTCATATTTCCAGTCTGCCTC | Antisense | 5422 |
| 227346_at | 628 | 977 | 618 | TCCAGTCTGCCTCTATGATGATGTT | Antisense | 5423 |
| 227346_at | 207 | 583 | 664 | GAACAAGGGATGTACCACTGGAGGA | Antisense | 5424 |
| 227346_at | 682 | 647 | 692 | GAGTATCCTTTTGTACACATTTTGA | Antisense | 5425 |
| 227346_at | 65 | 277 | 717 | AATGCTTCTTCTGTAGTGATAGAAC | Antisense | 5426 |
| 227361_at | 28 | 1139 | 2610 | TTCCCAGGATTCTCTTTGGGGGTCA | Antisense | 5427 |
| 227361_at | 712 | 587 | 2668 | GAAACAGTTTCAGATCCTGCCAGGA | Antisense | 5428 |
| 227361_at | 1026 | 333 | 2738 | CAGTACGTATCTAAGATGCTGACAC | Antisense | 5429 |
| 227361_at | 489 | 509 | 2767 | GCTAATGTGACTTTTCAGCTTATCA | Antisense | 5430 |
| 227361_at | 800 | 207 | 2813 | AAAGGTGTGGTTAGATGTTTTCTCA | Antisense | 5431 |
| 227361_at | 890 | 353 | 2847 | CATTAATTTATCACTGAGTCTCATT | Antisense | 5432 |
| 227361_at | 147 | 651 | 2862 | GAGTCTCATTCAACCAAGTAATCTA | Antisense | 5433 |
| 227361_at | 389 | 541 | 2896 | GCAAATTCTAGCAGTATGTCTTCGA | Antisense | 5434 |
| 227361_at | 945 | 725 | 2909 | GTATGTCTTCGATAACTTGGATGTT | Antisense | 5435 |
| 227361_at | 1082 | 101 | 2966 | AGTATTTTGTCCTATGTATAACACA | Antisense | 5436 |
| 227361_at | 488 | 289 | 3031 | AATTCTGGTAATTCATACTATTTCT | Antisense | 5437 |
| 227550_at | 1032 | 931 | 1697 | TGTTGCATTTTTCACTCTTAACCCG | Antisense | 5438 |
| 227550_at | 1042 | 781 | 1725 | GTGTGTTTCAGCTTATGTTCGTTCT | Antisense | 5439 |
| 227550_at | 827 | 557 | 1781 | GAAGGCGGCTTCTGATTTTTAGGGT | Antisense | 5440 |
| 227550_at | 574 | 939 | 1828 | TGAGGGGTTCAGACACCCAGTCTCC | Antisense | 5441 |
| 227550_at | 1125 | 909 | 1865 | TGAGGGGTCGACTGAGCTTTGTTGA | Antisense | 5442 |
| 227550_at | 992 | 493 | 1912 | GCGGGTCTCCATGTTATATTGTGTG | Antisense | 5443 |
| 227550_at | 785 | 1157 | 1930 | TTGTGTGTTTACTGAGCTTCCCACT | Antisense | 5444 |
| 227550_at | 666 | 671 | 1962 | GATGACACATTTGTCCATCGTCCTG | Antisense | 5445 |
| 227550_at | 403 | 1001 | 1991 | TCTGCTTTCCAGAGGACACCGGAGC | Antisense | 5446 |
| 227550_at | 1158 | 503 | 2041 | GCTGCCTCACATAGCTGTTTTGCAA | Antisense | 5447 |
| 227550_at | 145 | 129 | 2189 | AGAATGTACATAGTCCCTTTGGCCA | Antisense | 5448 |
| 228017_s_at | 378 | 479 | 799 | GCCTGCGTAAGTGAGGAAACAGCTG | Antisense | 5449 |
| 228017_s_at | 374 | 939 | 810 | TGAGGAAACAGCTGATCCTGCTCCT | Antisense | 5450 |
| 228017_s_at | 441 | 377 | 847 | CTCAGCGACCGACCAGTGACAATGA | Antisense | 5451 |
| 228017_s_at | 979 | 151 | 858 | ACCAGTGACAATGACAGGAGCTCCC | Antisense | 5452 |
| 228017_s_at | 337 | 769 | 862 | GTGACAATGACAGGAGCTCCCAGGC | Antisense | 5453 |
| 228017_s_at | 1106 | 383 | 933 | CTGCCCTGGGTTCTAAGTACTGGAC | Antisense | 5454 |
| 228017_s_at | 317 | 487 | 935 | GCCCTGGGTTCTAAGTACTGGACAC | Antisense | 5455 |
| 228017_s_at | 154 | 807 | 940 | GGGTTCTAAGTACTGGACACCAGCC | Antisense | 5456 |
| 228017_s_at | 48 | 317 | 983 | CACGGCTGGCTGCAGCGTCAAGAGA | Antisense | 5457 |
| 228017_s_at | 987 | 495 | 987 | GCTGGCTGCAGCGTCAAGAGAGTTT | Antisense | 5458 |
| 228017_s_at | 1105 | 501 | 991 | GCTGCAGCGTCAAGAGAGTTTGTAA | Antisense | 5459 |
| 228071_at | 1072 | 79 | 679 | AGGAGTTGGTGGAGCTGATAGAGAA | Antisense | 5460 |
| 228071_at | 259 | 821 | 707 | GGTGCAGTGCAACGAAGGGGCTTAC | Antisense | 5461 |
| 228071_at | 2 | 951 | 714 | TGCAACGAAGGGGCTTACTTTTCTG | Antisense | 5462 |
| 228071_at | 410 | 1137 | 734 | TTCTGATGACATATACAAGGACACA | Antisense | 5463 |
| 228071_at | 563 | 661 | 791 | GAGGAAAATCTACACTGACCAATTA | Antisense | 5464 |
| 228071_at | 447 | 27 | 964 | ATAGGATTTGGAAGATGCTTTCAGA | Antisense | 5465 |
| 228071_at | 15 | 597 | 987 | GAAATATGGCATAGGTTTTTGTCGA | Antisense | 5466 |
| 228071_at | 260 | 877 | 994 | GGCATAGGTTTTTGTCGAAATGTAA | Antisense | 5467 |
| 228071_at | 651 | 1107 | 1020 | TTTTATTCTTCCTAATTTACTGTGA | Antisense | 5468 |
| 228071_at | 603 | 1013 | 1026 | TCTTCCTAATTTACTGTGATTTGTT | Antisense | 5469 |
| 228071_at | 599 | 681 | 1075 | GATAGTTAGAGAAATACCTCCTTCC | Antisense | 5470 |
| 228094_at | 149 | 803 | 193 | GGGAGAAACACATTTACTCCCCAAT | Antisense | 5471 |
| 228094_at | 1102 | 373 | 272 | CTACATGACCATGCACCCAGTTTGG | Antisense | 5472 |
| 228094_at | 156 | 85 | 306 | AGGTCAGATCGGAACAACTCACTTG | Antisense | 5473 |
| 228094_at | 371 | 559 | 381 | GAATGGAGAGTCCCTTCATCTCAGC | Antisense | 5474 |
| 228094_at | 758 | 1023 | 401 | TCAGCAGCGGTGGAGACTCTCTCCT | Antisense | 5475 |
| 228094_at | 352 | 381 | 443 | CTCTACCAGTGATTTCAGACTCCCG | Antisense | 5476 |
| 228094_at | 1 | 667 | 514 | GATGGAGAATTTGGAGCCTGGCAGA | Antisense | 5477 |
| 228094_at | 364 | 109 | 538 | AGAGACTGGACAGCTCTGGAGGAAC | Antisense | 5478 |
| 228094_at | 295 | 997 | 552 | TCTGGAGGAACAGGCCTGCTGAGGG | Antisense | 5479 |
| 228094_at | 144 | 891 | 594 | GGCCTCTGGAGTGGGACACTGGCCC | Antisense | 5480 |
| 228094_at | 640 | 439 | 671 | CCTGTGGGCAGGGTTCTTAGTGGAT | Antisense | 5481 |
| 228786_at | 359 | 309 | 317 | CACACGCAGATCCTGCACTGTACTG | Antisense | 5482 |
| 228786_at | 819 | 49 | 326 | ATCCTGCACTGTACTGTCCAGTCAA | Antisense | 5483 |
| 228786_at | 1007 | 165 | 338 | ACTGTCCAGTCAACTGACTTCAGCA | Antisense | 5484 |
| 228786_at | 645 | 665 | 365 | GATGTCTGGGTTCAGCAGCGAGTTG | Antisense | 5485 |
| 228786_at | 1099 | 645 | 384 | GAGTTGCTGTGTGAGGCCACCAGAA | Antisense | 5486 |
| 228786_at | 198 | 803 | 494 | GGGAGACATTCAGAATCGAGGCGAA | Antisense | 5487 |
| 228786_at | 807 | 447 | 524 | CCTCAGTGCTCCTCTTGGAGGCAGA | Antisense | 5488 |
| 228786_at | 699 | 767 | 583 | GTGAATGTGGAAGACCAGCAGATTG | Antisense | 5489 |
| 228786_at | 1088 | 785 | 665 | GTGGTGAAGTGGCCCTGCACAAAAC | Antisense | 5490 |
| 228786_at | 679 | 263 | 671 | AAGTGGCCCTGCACAAAACGTTGCT | Antisense | 5491 |
| 228786_at | 332 | 1071 | 817 | TAGTAAAGCTAAGTTTTATCTCTTC | Antisense | 5492 |
| 228812_at | 843 | 637 | 765 | GAGACTTCATTGGTGATACACTCAA | Antisense | 5493 |
| 228812_at | 406 | 775 | 777 | GTGATACACTCAATTTTTACTGGGT | Antisense | 5494 |
| 228812_at | 488 | 509 | 807 | GCTAATAATGTTGGTCACTGTCTCA | Antisense | 5495 |
| 228812_at | 742 | 301 | 869 | CACAAAGCTTTTGGGTAACCAGCGT | Antisense | 5496 |
| 228812_at | 873 | 805 | 881 | GGGTAACCAGCGTTCTTAAATGTAT | Antisense | 5497 |
| 228812_at | 552 | 1075 | 903 | TATGGTTTTTGACCAGGTGAACCCT | Antisense | 5498 |
| 228812_at | 775 | 623 | 913 | GACCAGGTGAACCCTTTAGAAGTGA | Antisense | 5499 |
| 228812_at | 1155 | 199 | 963 | AAATACCTTTGGCTGTGATGAATGT | Antisense | 5500 |
| 228812_at | 1101 | 953 | 981 | TGAATGTAGATCCCAGCAGAATACC | Antisense | 5501 |
| 228812_at | 137 | 1153 | 1021 | TTGACTGAGTATTTGTAGATGCTTA | Antisense | 5502 |
| 228812_at | 198 | 259 | 1091 | AAGTTCTCAGGTACTGTTCAATTAT | Antisense | 5503 |
| 229152_at | 192 | 953 | 20 | TGAAGAAAGTTCTCCTCCTGATCAC | Antisense | 5504 |
| 229152_at | 858 | 387 | 34 | CTCCTGATCACAGCCATCTTGGCAG | Antisense | 5505 |
| 229152_at | 299 | 455 | 47 | CCATCTTGGCAGTGGCTGTTGGTTT | Antisense | 5506 |
| 229152_at | 343 | 603 | 115 | GACAGCGATGAATTAGCTTCAGGGT | Antisense | 5507 |
| 229152_at | 793 | 975 | 177 | TCCACCAATTCCATTTCCAAGATTT | Antisense | 5508 |
| 229152_at | 136 | 257 | 195 | AAGATTTCCATGGTTTAGACGTAAT | Antisense | 5509 |
| 229152_at | 502 | 1067 | 210 | TAGACGTAATTTTCCTATTCCAATA | Antisense | 5510 |
| 229152_at | 194 | 369 | 224 | CTATTCCAATACCTGAATCTGCCCC | Antisense | 5511 |
| 229152_at | 124 | 209 | 287 | AAAGTCACGATAAACCTGGTCACCT | Antisense | 5512 |
| 229152_at | 1068 | 199 | 382 | AAATAGCACACAGCATTCTCTAGTC | Antisense | 5513 |
| 229152_at | 142 | 25 | 408 | ATATCTTTAGTGATCTTCTTTAATA | Antisense | 5514 |
| 229598_at | 248 | 15 | 566 | ATTTTTCTTAGCTAAATCTGGCAAC | Antisense | 5515 |
| 229598_at | 573 | 133 | 636 | AGCAGAATCTGCCATATGAGTAATA | Antisense | 5516 |
| 229598_at | 259 | 649 | 653 | GAGTAATAGAAGTGAGCAGGCCCAG | Antisense | 5517 |
| 229598_at | 304 | 335 | 675 | CAGGACTCCCTAAGTCAAGAAACCA | Antisense | 5518 |
| 229598_at | 19 | 121 | 692 | AGAAACCAAGAGGCGTCATTACGGA | Antisense | 5519 |
| 229598_at | 549 | 209 | 717 | AAAGAGTAACTCACCCTGTGTGCTC | Antisense | 5520 |
| 229598_at | 90 | 143 | 759 | AGCGATGCCCCCATGTTATGAATGG | Antisense | 5521 |
| 229598_at | 747 | 859 | 784 | GGAAAAGTTCACTGAAGGGTTCATA | Antisense | 5522 |
| 229598_at | 796 | 1161 | 846 | TTGGATACCTTCAAGGGATCAGAAA | Antisense | 5523 |
| 229598_at | 394 | 1017 | 899 | TCATCATTACTCTCTACATTATTAT | Antisense | 5524 |
| 229598_at | 448 | 1109 | 983 | TTTTAACTTGTCTGCATGTTTGAAA | Antisense | 5525 |
| 231229_at | 843 | 533 | 31 | GCACGTCCAAGGTGATCCTGAGGGC | Antisense | 5526 |
| 231229_at | 969 | 317 | 96 | CACCCTGAAGAAGGCTGTTTCCACC | Antisense | 5527 |
| 231229_at | 999 | 605 | 130 | GACATGGCCCGAAATGCCTATCACT | Antisense | 5528 |
| 231229_at | 632 | 477 | 145 | GCCTATCACTTCAAGCGTGTGCTCA | Antisense | 5529 |
| 231229_at | 105 | 389 | 247 | CTCCAAGTCCAAGCTCAAGGTCAAG | Antisense | 5530 |
| 231229_at | 658 | 603 | 315 | GACAGCACAGGTCACTACTGGGCTC | Antisense | 5531 |
| 231229_at | 453 | 1057 | 330 | TACTGGGCTCCAAACAGGGGCACAA | Antisense | 5532 |
| 231229_at | 908 | 293 | 352 | CAAGCGGCTTATCAAGGGGGTTCGA | Antisense | 5533 |
| 231229_at | 961 | 803 | 369 | GGGTTCGAAGGGTGGCCAAGTGCCA | Antisense | 5534 |
| 231229_at | 847 | 59 | 403 | ATGAGGCAGGCCAGGCAAGCAGTCA | Antisense | 5535 |
| 231229_at | 1130 | 419 | 441 | CGCCATTGGCTCAGTGCAGTGGGAA | Antisense | 5536 |
| 231929_at | 1064 | 879 | 2289 | GGCTCAAATACCATACCTCAGAAAA | Antisense | 5537 |
| 231929_at | 779 | 587 | 2336 | GAAACAGTCTTTGCAGCTGTGTGAC | Antisense | 5538 |
| 231929_at | 711 | 533 | 2348 | GCAGCTGTGTGACAAGTCACTCTAC | Antisense | 5539 |
| 231929_at | 1117 | 195 | 2359 | ACAAGTCACTCTACTACATACTGAT | Antisense | 5540 |
| 231929_at | 1014 | 165 | 2378 | ACTGATTTGGAGACCTCCGCTAAAT | Antisense | 5541 |
| 231929_at | 912 | 621 | 2389 | GACCTCCGCTAAATAGTTTTATCAC | Antisense | 5542 |
| 231929_at | 855 | 167 | 2412 | ACTGCAGACTAAAATGTGGGACTTG | Antisense | 5543 |
| 231929_at | 472 | 205 | 2606 | AAAGGTTTGTACTGTCTGAGTGCAC | Antisense | 5544 |
| 231929_at | 986 | 753 | 2619 | GTCTGAGTGCACAGCTACTGGAATA | Antisense | 5545 |
| 231929_at | 1101 | 1117 | 2647 | TTAGGGAATCTCAGGAACAAGCATA | Antisense | 5546 |
| 231929_at | 63 | 741 | 2705 | GTAAGTGCAGTTTTTAATTCTGTAT | Antisense | 5547 |
| 232311_at | 229 | 181 | 2003 | ACATACCTTGGGTTGATCCACTTAG | Antisense | 5548 |
| 232311_at | 757 | 675 | 2017 | GATCCACTTAGGAACCTCAGATAAT | Antisense | 5549 |
| 232311_at | 930 | 231 | 2042 | AACATCTGCCACGTATAGAGCAATT | Antisense | 5550 |
| 232311_at | 444 | 1071 | 2069 | TATGTCCCAGGCACTCTACTAGACA | Antisense | 5551 |
| 232311_at | 999 | 371 | 2084 | CTACTAGACACTTCATACAGTTTAG | Antisense | 5552 |
| 232311_at | 33 | 811 | 2119 | GGGTGTAGATCAAGGCAGGAGCAGG | Antisense | 5553 |
| 232311_at | 387 | 667 | 2220 | GATGGGGGCTATTATGAACTGAGAA | Antisense | 5554 |
| 232311_at | 683 | 1159 | 2264 | TTGGGGCCAAATCATGTAGACTCTT | Antisense | 5555 |
| 232311_at | 543 | 1067 | 2280 | TAGACTCTTGAGTGATGTGTTAAGG | Antisense | 5556 |
| 232311_at | 137 | 869 | 2303 | GGAATGCTATGAGTGCTGAGAGGGC | Antisense | 5557 |
| 232311_at | 1085 | 521 | 2326 | GCATCAGAAGTCCTTGAGAGCCTCC | Antisense | 5558 |
| 232843_s_at | 48 | 299 | 4483 | CAAGCTAAAAGAGAACCTCAGGCCA | Antisense | 5559 |
| 232843_s_at | 143 | 575 | 4495 | GAACCTCAGGCCAATGATCGAGCGG | Antisense | 5560 |
| 232843_s_at | 211 | 891 | 4503 | GGCCAATGATCGAGCGGAAAATTCC | Antisense | 5561 |
| 232843_s_at | 944 | 859 | 4518 | GGAAAATTCCAGAACTGTACAAGCC | Antisense | 5562 |
| 232843_s_at | 31 | 737 | 4534 | GTACAAGCCAATATTCAGAGTTGAG | Antisense | 5563 |
| 232843_s_at | 479 | 125 | 4568 | AGAACATTGTGCTGTCTGTCAGCAT | Antisense | 5564 |
| 232843_s_at | 265 | 499 | 4578 | GCTGTCTGTCAGCATATGTATATCA | Antisense | 5565 |
| 232843_s_at | 683 | 745 | 4585 | GTCAGCATATGTATATCAGCTACAA | Antisense | 5566 |
| 232843_s_at | 658 | 23 | 4611 | ATATATTCAACTTTGACTTCTTTTG | Antisense | 5567 |
| 232843_s_at | 920 | 1125 | 4696 | TTAATATGACTGTGACCTTGACTGA | Antisense | 5568 |
| 232843_s_at | 86 | 1079 | 4700 | TATGACTGTGACCTTGACTGATAAT | Antisense | 5569 |
| 235276_at | 927 | 159 | 169 | ACCCTGCACTCCCAAAGATTTTGTG | Antisense | 5570 |
| 235276_at | 224 | 105 | 184 | AGATTTTGTGCAGATGGGTAGTTCC | Antisense | 5571 |
| 235276_at | 950 | 769 | 241 | GTGACTTACTTCATGACCAGAACTA | Antisense | 5572 |
| 235276_at | 28 | 809 | 281 | GGGGTATAAACATCTTGCTTAACCA | Antisense | 5573 |
| 235276_at | 646 | 121 | 343 | AGACTTGCTGCCTAAAGGAGCCCAC | Antisense | 5574 |
| 235276_at | 925 | 969 | 351 | TGCCTAAAGGAGCCCACCATTTTAC | Antisense | 5575 |
| 235276_at | 932 | 175 | 374 | ACTTTTCACATTTAATCTGCCACGT | Antisense | 5576 |
| 235276_at | 513 | 1125 | 385 | TTAATCTGCCACGTTGAATCAATTG | Antisense | 5577 |
| 235276_at | 1070 | 213 | 415 | AAACCTGACTCGCAGGTGACTGGAC | Antisense | 5578 |
| 235276_at | 134 | 815 | 429 | GGTGACTGGACAGGAAATCCCAAAG | Antisense | 5579 |
| 235276_at | 675 | 305 | 449 | CAAAGTTCCACCATTTCTATGCTTA | Antisense | 5580 |
| 235391_at | 477 | 17 | 281 | ATTTCACGGCAAAGCTTTAGAGGTC | Antisense | 5581 |
| 235391_at | 482 | 1101 | 296 | TTTAGAGGTCTACACTGCTGCCTAC | Antisense | 5582 |
| 235391_at | 458 | 1051 | 306 | TACACTGCTGCCTACCAGAATATAC | Antisense | 5583 |
| 235391_at | 862 | 289 | 369 | AATTCTCTGTATGCACCAGATTATT | Antisense | 5584 |
| 235391_at | 758 | 541 | 417 | GCAAATTCAAAGTCACCTCTTCAGA | Antisense | 5585 |
| 235391_at | 16 | 155 | 431 | ACCTCTTCAGAGATCACTGTCAGCT | Antisense | 5586 |
| 235391_at | 439 | 167 | 446 | ACTGTCAGCTAAGTGTGTATCTGGA | Antisense | 5587 |
| 235391_at | 569 | 185 | 476 | ACAGGTATCCACTTGTCGACTAAGA | Antisense | 5588 |
| 235391_at | 29 | 1123 | 565 | TTAAGTAAACTACACATTTCCATTT | Antisense | 5589 |
| 235391_at | 985 | 579 | 630 | GAACTTTATACTCACTTTGCTATGT | Antisense | 5590 |
| 235391_at | 369 | 245 | 656 | AAGCCTCAAAGTGAAGTCCAACTGG | Antisense | 5591 |
| 235421_at | 755 | 29 | 109 | ATACCTTCACAATCTGAACTTTCTC | Antisense | 5592 |
| 235421_at | 770 | 579 | 124 | GAACTTTCTCTAGATGGGCACAGAT | Antisense | 5593 |
| 235421_at | 627 | 95 | 201 | AGTGCTGAGGCTTTTTGCAGGCGCA | Antisense | 5594 |
| 235421_at | 682 | 957 | 216 | TGCAGGCGCAGTTAGCAGGCGAACT | Antisense | 5595 |
| 235421_at | 1114 | 883 | 233 | GGCGAACTGGTCAGCAGGGTTTCTG | Antisense | 5596 |
| 235421_at | 1015 | 409 | 263 | CGTAGATTTGCGTGACAGGGGCCCT | Antisense | 5597 |
| 235421_at | 1142 | 337 | 278 | CAGGGGCCCTGTGTTGCTAAGGAAT | Antisense | 5598 |
| 235421_at | 900 | 223 | 319 | AAAAGTCTCAGCGTCAGACACTCCT | Antisense | 5599 |
| 235421_at | 849 | 449 | 376 | CCTTTCCAGGCAACTCACAGTTTAG | Antisense | 5600 |
| 235421_at | 331 | 697 | 408 | GTTGTGTGTGTTCAAGCTCTTGGGT | Antisense | 5601 |
| 235421_at | 1103 | 231 | 477 | AACATCTTGTCTTACAGCTCTTTTA | Antisense | 5602 |
| 235688_s_at | 55 | 939 | 27 | TGAGGCAGTGCATGTTCTTGGCCCA | Antisense | 5603 |
| 235688_s_at | 722 | 895 | 45 | TGGCCCAGAGTAAGTGCTTAGTGAA | Antisense | 5604 |
| 235688_s_at | 52 | 91 | 64 | AGTGAATGCTTTCTAACTCCGAACC | Antisense | 5605 |
| 235688_s_at | 1104 | 809 | 108 | GGGTGTTGAGCAAAAGGGGCCTTCA | Antisense | 5606 |
| 235688_s_at | 1084 | 813 | 123 | GGGGCCTTCAAGATGTTCAAGGCAC | Antisense | 5607 |
| 235688_s_at | 178 | 891 | 186 | GGCCTCAGTGGGTGCATGTGATTAT | Antisense | 5608 |
| 235688_s_at | 45 | 935 | 204 | TGATTATCCACGTTTCACCTATGAA | Antisense | 5609 |
| 235688_s_at | 773 | 601 | 276 | GACAGGGCCTCGATTCTGTTTTAAA | Antisense | 5610 |
| 235688_s_at | 678 | 739 | 290 | TCTGTTTTAAACTCCAGTAGTCCCT | Antisense | 5611 |
| 235688_s_at | 1024 | 443 | 396 | CCTCCATTACCGTCACTGGTGAAAT | Antisense | 5612 |
| 235688_s_at | 1100 | 165 | 410 | ACTGGTGAAATGCGGCTCACCTCCC | Antisense | 5613 |
| 236203_at | 84 | 63 | 21 | ATGTCAGGTTTGTACCTACCACATT | Antisense | 5614 |
| 236203_at | 116 | 873 | 107 | GGCACATAGTAAGTAGTCGATAGGT | Antisense | 5615 |
| 236203_at | 194 | 3 | 148 | ATTGTTATTTTCTGGAGTCCAACTA | Antisense | 5616 |
| 236203_at | 963 | 647 | 162 | GAGTCCAACTAACAAATCCCACAGT | Antisense | 5617 |
| 236203_at | 211 | 203 | 175 | AAATCCCACAGTGAATGACATCACA | Antisense | 5618 |
| 236203_at | 87 | 803 | 200 | GGGATGCAACCAACAAGATCCAGAA | Antisense | 5619 |
| 236203_at | 252 | 177 | 232 | ACTTCTACTAGATAAACAACTCCAT | Antisense | 5620 |
| 236203_at | 12 | 683 | 242 | GATAAACAACTCCATTTCTTCAGCA | Antisense | 5621 |
| 236203_at | 472 | 573 | 326 | GAACCAAATTTGTATGAGGCAATCA | Antisense | 5622 |
| 236203_at | 990 | 221 | 353 | AAAACTGACACCGACTGTATTAAGG | Antisense | 5623 |
| 236203_at | 919 | 59 | 401 | ATGAGATTGCTGTTATGTTTTCTAA | Antisense | 5624 |
| 236627_at | 256 | 235 | 92 | AACTAGATTTTTGACCCTCACTCAT | Antisense | 5625 |
| 236627_at | 901 | 367 | 121 | CTTTCCTTGCTATAGACATGCTTTG | Antisense | 5626 |
| 236627_at | 995 | 119 | 134 | AGACATGCTTTGCATACCCAGCTTC | Antisense | 5627 |
| 236627_at | 1111 | 1133 | 156 | TTCTAGCCACATCTTCCCTAAGAAA | Antisense | 5628 |
| 236627_at | 190 | 839 | 230 | GGATCTGCATGTTGTCAGCTACCTA | Antisense | 5629 |
| 236627_at | 326 | 697 | 240 | GTTGTCAGCTACCTAAAGCAGCAAA | Antisense | 5630 |
| 236627_at | 400 | 1045 | 281 | TAAAATCAGGGAACCTTGCAGTTTA | Antisense | 5631 |
| 236627_at | 701 | 243 | 292 | AACCTTGCAGTTTAAGGCACACCTT | Antisense | 5632 |
| 236627_at | 1156 | 1101 | 329 | TTTAATCCTTCAATGCATCCAGCCA | Antisense | 5633 |
| 236627_at | 1062 | 979 | 334 | TCCTTCAATGCATCCAGCCAAAACA | Antisense | 5634 |
| 236627_at | 149 | 1063 | 375 | TACCATCCATCAATGCAAGGTTTTT | Antisense | 5635 |
| 236681_at | 179 | 589 | 250 | GAAACTCATGTGTCCTCATGGATCG | Antisense | 5636 |
| 236681_at | 133 | 343 | 266 | CATGGATCGTGGATGCCTTCATTTC | Antisense | 5637 |
| 236681_at | 1146 | 1021 | 326 | TCAGTAGAGTGACCCGCGGATGGCA | Antisense | 5638 |
| 236681_at | 447 | 69 | 345 | ATGGCATAAATGCACCTCCTTTTCT | Antisense | 5639 |
| 236681_at | 729 | 1015 | 367 | TCTTGGCCTTGGATCTATGGGTCTG | Antisense | 5640 |
| 236681_at | 409 | 835 | 392 | GGATTGTGGTCATCTCCTCAATCCT | Antisense | 5641 |
| 236681_at | 882 | 501 | 426 | GCTGAATCAATGTGGCCGTGGGTGG | Antisense | 5642 |
| 236681_at | 121 | 889 | 439 | GGCCGTGGGTGGGAACTTACATACA | Antisense | 5643 |
| 236681_at | 896 | 613 | 482 | GACTGTCTAAACAAGGGGGCCTCGC | Antisense | 5644 |
| 236681_at | 1013 | 789 | 498 | GGGCCTCGCATGGAGCTGTAAAGCA | Antisense | 5645 |
| 236681_at | 522 | 805 | 669 | GGGATACATGGGCAGTGCTTCAGAC | Antisense | 5646 |
| 238834_at | 67 | 641 | 19 | GAGAGAATCCTGCATCTAACAATTT | Antisense | 5647 |
| 238834_at | 550 | 1079 | 59 | TATGCTGTTATTCCTTACCTAGAGA | Antisense | 5648 |
| 238834_at | 701 | 1067 | 78 | TAGAGAAACAATTTCCCTCCAAAGT | Antisense | 5649 |
| 238834_at | 673 | 1125 | 107 | TTGAGGGGTCTGTTTAGGCCAGGCC | Antisense | 5650 |
| 238834_at | 460 | 71 | 122 | AGGCCAGGCCAACACAAGTGACCTA | Antisense | 5651 |
| 238834_at | 467 | 1095 | 220 | TTTGCTGGTGTGTACTTAGCTCAGA | Antisense | 5652 |
| 238834_at | 525 | 697 | 262 | GTTGGGTCAACTATTTCTAATGGGA | Antisense | 5653 |
| 238834_at | 95 | 801 | 283 | GGGACTTTTCCATTTGCATGTACAG | Antisense | 5654 |
| 238834_at | 189 | 99 | 306 | AGTCACTGGAAACTGCTGGGCAGAG | Antisense | 5655 |
| 238834_at | 798 | 189 | 354 | ACACTTTTTCCACCTGTCAGATTGG | Antisense | 5656 |
| 238834_at | 672 | 909 | 398 | TGGGGAAAATGGCATTCTCCCACTT | Antisense | 5657 |
| 241671_x_at | 579 | 1 | 28 | ATTCTGAAGCCAGACCGTCTTTCCT | Antisense | 5658 |
| 241671_x_at | 716 | 993 | 223 | TCGCAGCTGGGCGAGGGGACTTGGA | Antisense | 5659 |
| 241671_x_at | 861 | 403 | 229 | CTGGGCGAGGGGACTTGGAGGACAG | Antisense | 5660 |
| 241671_x_at | 555 | 813 | 237 | GGGGACTTGGAGGACAGGGTGAAGC | Antisense | 5661 |
| 241671_x_at | 958 | 177 | 241 | ACTTGGAGGACAGGGTGAAGCTGCA | Antisense | 5662 |
| 241671_x_at | 834 | 951 | 256 | TGAAGCTGCAGAAGACCTGGGGTGG | Antisense | 5663 |
| 241671_x_at | 1116 | 501 | 260 | GCTGCAGAAGACCTGGGGTGGGATG | Antisense | 5664 |
| 241671_x_at | 425 | 1067 | 287 | TAGAGAGGACGCCAAGGACTGGGGA | Antisense | 5665 |
| 241671_x_at | 19 | 113 | 290 | AGAGGACGCCAAGGACTGGGGAAGG | Antisense | 5666 |
| 241671_x_at | 516 | 865 | 315 | GGAAGTTAGGAATACCTTACATCCA | Antisense | 5667 |
| 241671_x_at | 536 | 75 | 322 | AGGAATACCTTACATCCAATGCCCA | Antisense | 5668 |
| 243010_at | 200 | 597 | 608 | GAAATACACCCACTCTCTTGGAATA | Antisense | 5669 |
| 243010_at | 1151 | 57 | 633 | ATGACGTACCACTCAGTTGGACCCT | Antisense | 5670 |
| 243010_at | 856 | 619 | 652 | GACCCTCAAGAGTCACTGCTTTGTC | Antisense | 5671 |
| 243010_at | 506 | 417 | 704 | CGCACGCTTCCATTTGATGCATTTG | Antisense | 5672 |
| 243010_at | 1129 | 63 | 753 | ATGTCATTGTCCTTGAGACCCTACA | Antisense | 5673 |
| 243010_at | 296 | 639 | 767 | GAGACCCTACATGTGCAGTTTGGCT | Antisense | 5674 |
| 243010_at | 825 | 1097 | 840 | TTTCCTGCAGGCTTTTCCATGAGTA | Antisense | 5675 |
| 243010_at | 970 | 579 | 879 | GAACAAATCTGTATGGCTTTTCCCC | Antisense | 5676 |
| 243010_at | 483 | 767 | 943 | GTGAACTTGTCCTAGTATGCTTGCC | Antisense | 5677 |
| 243010_at | 1033 | 357 | 962 | CTTGCCTCACAAACGTTTTAGCCAT | Antisense | 5678 |
| 243010_at | 136 | 365 | 1119 | CTTAGCCTGACAGTGTCCTGTTCTC | Antisense | 5679 |
| 244061_at | 165 | 595 | 45 | GAAATGGCACATTTTCTGGATGTGA | Antisense | 5680 |
| 244061_at | 750 | 729 | 140 | GTACTTACCCTCATAAGCATTAAAA | Antisense | 5681 |
| 244061_at | 603 | 597 | 178 | GAAATTCCATAGAAATGTGCCTATC | Antisense | 5682 |
| 244061_at | 238 | 763 | 194 | GTGCCTATCATGTTATACTGACTCA | Antisense | 5683 |
| 244061_at | 337 | 305 | 217 | CAAACCAGAAGACCTAGAGTATGAT | Antisense | 5684 |
| 244061_at | 82 | 783 | 260 | GTGGTGGGTATGAGTGGAAGTATGT | Antisense | 5685 |
| 244061_at | 152 | 783 | 283 | GTGTGTGAGATTTATCATTGCCATA | Antisense | 5686 |
| 244061_at | 346 | 645 | 289 | GAGATTTATCATTGCCATAGTGTAA | Antisense | 5687 |
| 244061_at | 554 | 737 | 310 | GTAAAAGAGTTGAATTAGCTTCCAC | Antisense | 5688 |
| 244061_at | 103 | 13 | 323 | ATTAGCTTCCACTTGACTAGATGAG | Antisense | 5689 |
| 244061_at | 192 | 1069 | 340 | TAGATGAGAGCTCTTAGTTCTTATT | Antisense | 5690 |
| 244393_x_at | 880 | 421 | 132 | CCCAGCCGCTATAACTTTTAACAAT | Antisense | 5691 |
| 244393_x_at | 314 | 197 | 152 | ACAATTCCCATATGTCCTTTATTCC | Antisense | 5692 |
| 244393_x_at | 700 | 1077 | 162 | TATGTCCTTTATTCCACTAAGATGA | Antisense | 5693 |
| 244393_x_at | 464 | 59 | 183 | ATGAGTGCAGTATATATTTCCATCT | Antisense | 5694 |
| 244393_x_at | 489 | 23 | 196 | ATATTTCCATCTGTCCAAGGCTTCC | Antisense | 5695 |
| 244393_x_at | 796 | 795 | 270 | GGGCATTTGGGGAATCCAGGCTGTG | Antisense | 5696 |
| 244393_x_at | 1140 | 455 | 285 | CCAGGCTGTGATTCAGGGAAGTTCC | Antisense | 5697 |
| 244393_x_at | 597 | 257 | 303 | AAGTTCCAAGTGTCTGATGAAGTGT | Antisense | 5698 |
| 244393_x_at | 722 | 1155 | 328 | TTGTTTTACATCTTTGTGTCCCTTG | Antisense | 5699 |
| 244393_x_at | 247 | 453 | 348 | CCTTGCAGGTCTAGCACTGTGCTAT | Antisense | 5700 |
| 244393_x_at | 748 | 765 | 366 | GTGCTATGTAGGTAACATGTGCTCC | Antisense | 5701 |
| 34210_at | 633 | 1033 | 46 | TAATCGGCTCACTATAGGAATTTGC | Antisense | 5702 |
| 34210_at | 264 | 249 | 103 | AAGCAGCTAAACCAAAAGAAGCCTC | Antisense | 5703 |
| 34210_at | 565 | 331 | 240 | CAGATACAAACTGGACTCTCAGGAC | Antisense | 5704 |
| 34210_at | 524 | 251 | 246 | CAAACTGGACTCTCAGGACAAAACG | Antisense | 5705 |
| 34210_at | 220 | 1021 | 258 | TCAGGACAAAACGACACCAGCCAAA | Antisense | 5706 |
| 34210_at | 394 | 1145 | 324 | TTCCTTTTCTTCGTGGCCAATGCCA | Antisense | 5707 |
| 34210_at | 371 | 1133 | 330 | TTCTTCGTGGCCAATGCCATAATCC | Antisense | 5708 |
| 34210_at | 655 | 1137 | 360 | TTCTGCTTCAGTTGAGGTGACACGT | Antisense | 5709 |
| 34210_at | 600 | 1127 | 366 | TTCAGTTGAGGTGACACGTCTCAGC | Antisense | 5710 |
| 34210_at | 921 | 769 | 376 | GTGACACGTCTCAGCCTTAGCCCTG | Antisense | 5711 |
| 34210_at | 1002 | 833 | 460 | GGTTGATGCCAGACATCACCAGGTT | Antisense | 5712 |
| 34210_at | 367 | 39 | 474 | ATCACCAGGTTGTAGAAGTTGACAG | Antisense | 5713 |
| 34210_at | 346 | 321 | 476 | CACCAGGTTGTAGAAGTTGACAGGC | Antisense | 5714 |
| 34210_at | 615 | 63 | 539 | ATGTAGGGGCCAAGCAGTGCCCAGC | Antisense | 5715 |
| 34210_at | 410 | 925 | 540 | TGTAGGGGCCAAGCAGTGCCCAGCT | Antisense | 5716 |
| 34210_at | 394 | 741 | 569 | GTCAATAAAGTTACCCTTGTACTTG | Antisense | 5717 |

TABLE 18
34PS gene list probe sequences

| | | | | | | |
|---|---|---|---|---|---|---|
| | | | Probe | | | |
| Probe Set | | | Interrogation | | Target | SEQ ID |
| Name | Probe.X | Probe.Y | Position | Probe Sequence | Strandedness | NO: |

| | | | | | | |
|---|---|---|---|---|---|---|
| 1552584_at | 846 | 165 | 1489 | ACTGTGGCCGACCTACTATTTTTAT | Antisense | 5718 |
| 1552584_at | 300 | 833 | 1527 | GGTTCTCAGTCTGTTGGCAGACTGG | Antisense | 5719 |
| 1552584_at | 242 | 97 | 1534 | AGTCTGTTGGCAGACTGGAGTGCAA | Antisense | 5720 |
| 1552584_at | 900 | 145 | 1796 | AGCCTGGTCCCATATCATAGTGAAA | Antisense | 5721 |
| 1552584_at | 667 | 351 | 1806 | CATATCATAGTGAAATGGTGCCTGT | Antisense | 5722 |
| 1552584_at | 458 | 205 | 1818 | AAATGGTGCCTGTAAAGCTCTCAGC | Antisense | 5723 |
| 1552584_at | 742 | 247 | 1832 | AAGCTCTCAGCATTGGCTTGGCACA | Antisense | 5724 |
| 1552584_at | 952 | 1161 | 1844 | TTGGCTTGGCACATGCAGTTGGTAC | Antisense | 5725 |
| 1552584_at | 153 | 873 | 1851 | GGCACATGCAGTTGGTACTCAATAA | Antisense | 5726 |
| 1552584_at | 441 | 827 | 1864 | GGTACTCAATAAACGGCTGTTGCTA | Antisense | 5727 |
| 1552584_at | 389 | 1033 | 1869 | TCAATAAACGGCTGTTGCTATCCCC | Antisense | 5728 |
| 1558972_s_at | 492 | 1125 | 3472 | TTCTAAGACCCACATTTGGTTATTG | Antisense | 5729 |
| 1558972_s_at | 889 | 1161 | 3487 | TTGGTTATTGAAGGCCACAGCGAAT | Antisense | 5730 |
| 1558972_s_at | 756 | 469 | 3506 | GCGAATCTTAACCTAACAGCCTTGA | Antisense | 5731 |
| 1558972_s_at | 10 | 323 | 3522 | CAGCCTTGACAAACTGCACCATAGG | Antisense | 5732 |
| 1558972_s_at | 1127 | 535 | 3537 | GCACCATAGGTGTTTTTAGACTCAT | Antisense | 5733 |
| 1558972_s_at | 345 | 1117 | 3639 | TTAGTAACTACCATTCTTTGATTAG | Antisense | 5734 |
| 1558972_s_at | 1154 | 555 | 3674 | GAATGCATATCTTACTTTGGTTGTA | Antisense | 5735 |
| 1558972_s_at | 1071 | 717 | 3692 | GGTTGTAAATTATCAAGGGCTTTCT | Antisense | 5736 |
| 1558972_s_at | 151 | 1029 | 3756 | TCACATACTGTGTTTCCAGTTGTCT | Antisense | 5737 |
| 1558972_s_at | 755 | 777 | 3765 | GTGTTTCCAGTTGTCTTGATATTGA | Antisense | 5738 |
| 1558972_s_at | 500 | 925 | 3794 | TGTAATAAACTTCATGCTCACCTAT | Antisense | 5739 |
| 1559584_a_at | 971 | 7 | 2313 | ATTCCTGGGCTCGAGAGAGCCACTC | Antisense | 5740 |
| 1559584_a_at | 263 | 775 | 2412 | GTGATTTTGCCATATTAGTTTACCA | Antisense | 5741 |
| 1559584_a_at | 986 | 1095 | 2417 | TTTGCCATATTAGTTTACCATCTGT | Antisense | 5742 |
| 1559584_a_at | 183 | 715 | 2429 | GTTTACCATCTGTACAATTATTTGC | Antisense | 5743 |
| 1559584_a_at | 262 | 455 | 2434 | CCATCTGTACAATTATTTGCTTTAT | Antisense | 5744 |
| 1559584_a_at | 10 | 351 | 2465 | CTTTAAATTGGCTCATTATTCACTT | Antisense | 5745 |
| 1559584_a_at | 197 | 293 | 2470 | AATTGGCTCATTATTCACTTAATGA | Antisense | 5746 |
| 1559584_a_at | 289 | 597 | 2493 | GAAATTATTGCAACAGGAATCTTTT | Antisense | 5747 |
| 1559584_a_at | 590 | 543 | 2502 | GCAACAGGAATCTTTTGTATCACTT | Antisense | 5748 |
| 1559584_a_at | 1055 | 553 | 2509 | GAATCTTTTGTATCACTTCTGTAAA | Antisense | 5749 |
| 1559584_a_at | 1061 | 751 | 2544 | GTCTCATTTGCCATCAATAGAAGGT | Antisense | 5750 |
| 1563473_at | 8 | 583 | 5590 | GAAAATTCCTGGCAGTTTCAACTGT | Antisense | 5751 |
| 1563473_at | 504 | 1067 | 5617 | TAGACATTGCTAACCTGTTCTCCAA | Antisense | 5752 |
| 1563473_at | 966 | 499 | 5646 | GCTGAACCAATTTCTGTTTCCTCAA | Antisense | 5753 |
| 1563473_at | 149 | 233 | 5757 | AACATTTCTGCATAAGGTTCTACAG | Antisense | 5754 |
| 1563473_at | 252 | 1079 | 5796 | TATGACCCTTTGGATTATGCCTACA | Antisense | 5755 |
| 1563473_at | 686 | 235 | 5843 | AACTACATTGTACCTGGCCTTAGGC | Antisense | 5756 |
| 1563473_at | 408 | 203 | 5888 | AAATGAAACCAGCTTTTGCCCTCAG | Antisense | 5757 |
| 1563473_at | 538 | 1111 | 5901 | TTTTGCCCTCAGGTTGATCCCATCT | Antisense | 5758 |
| 1563473_at | 343 | 677 | 5916 | GATCCCATCTCCTGGAGTTGGCAGA | Antisense | 5759 |
| 1563473_at | 885 | 215 | 5966 | AAACTGTATGGTTCACATTGTGCTA | Antisense | 5760 |
| 1563473_at | 251 | 31 | 6065 | ATACCGTATGCATTATGTCCCAGGG | Antisense | 5761 |
| 203915_at | 1064 | 689 | 1973 | GATTATCAATTACCACACCATCTCC | Antisense | 5762 |
| 203915_at | 1124 | 569 | 2058 | GAAGCATGATTGGTGCCCAGTTAGC | Antisense | 5763 |
| 203915_at | 634 | 423 | 2073 | CCCAGTTAGCCTCTGCAGGATGTGG | Antisense | 5764 |
| 203915_at | 836 | 725 | 2131 | GTAGGAGAGGTTGTCTGTGGCCAGA | Antisense | 5765 |
| 203915_at | 555 | 153 | 2162 | ACCTATACTCACTTTCCCAAATTGA | Antisense | 5766 |
| 203915_at | 672 | 1151 | 2183 | TTGAATCACTGCTCACACTGCTGAT | Antisense | 5767 |
| 203915_at | 1055 | 1067 | 2212 | TAGAGTGCTGTCCGGTGGAGATCCC | Antisense | 5768 |
| 203915_at | 116 | 433 | 2235 | CCACCCGAACGTCTTATCTAATCAT | Antisense | 5769 |
| 203915_at | 256 | 41 | 2255 | ATCATGAAACTCCCTAGTTCCTTCA | Antisense | 5770 |
| 203915_at | 1156 | 701 | 2271 | GTTCCTTCATGTAACTTCCCTGAAA | Antisense | 5771 |
| 203915_at | 954 | 1093 | 2315 | TTTGAGAGTCTGTGACCCACTTACC | Antisense | 5772 |
| 204116_at | 475 | 1137 | 867 | TTCTGGCTGGAACGGACGATGCCCC | Antisense | 5773 |
| 204116_at | 606 | 821 | 917 | GGATCTTGTTACTGAATACCACGGG | Antisense | 5774 |
| 204116_at | 110 | 817 | 960 | GGTGTGTCTAAGGGACTGGCTGAGA | Antisense | 5775 |
| 204116_at | 902 | 173 | 997 | ACTACAGTGAACGACTCTGCCTCGT | Antisense | 5776 |
| 204116_at | 121 | 613 | 1009 | GACTCTGCCTCGTCAGTGAGATTCC | Antisense | 5777 |
| 204116_at | 998 | 707 | 1111 | GTTACACCCTAAAGCCTGAAACCTG | Antisense | 5778 |
| 204116_at | 845 | 241 | 1136 | AACCCCAATCCTCTGACAGAAGAAC | Antisense | 5779 |
| 204116_at | 309 | 819 | 1166 | GGTCCTGTAGCCCTAAGTGGTACTA | Antisense | 5780 |
| 204116_at | 1123 | 781 | 1182 | GTGGTACTAACTTTCCTTCATTCAA | Antisense | 5781 |
| 204116_at | 877 | 883 | 1242 | GGCTGATTTGGAATTTTGTGCCCCC | Antisense | 5782 |
| 204116_at | 453 | 285 | 1253 | AATTTTGTGCCCCCATGTAAGCACC | Antisense | 5783 |
| 204533_at | 589 | 1043 | 586 | TAACTCTACCCTGGCACTATAATGT | Antisense | 5784 |
| 204533_at | 638 | 507 | 613 | GCTCTACTGAGGTGCTATGTTCTTA | Antisense | 5785 |
| 204533_at | 218 | 1011 | 633 | TCTTAGTGGATGTTCTGACCCTGCT | Antisense | 5786 |
| 204533_at | 927 | 999 | 646 | TCTGACCCTGCTTCAAATATTTCCC | Antisense | 5787 |
| 204533_at | 268 | 827 | 692 | GGTACTAAGGAATCTTTCTGCTTTG | Antisense | 5788 |
| 204533_at | 206 | 475 | 711 | GCTTTGGGGTTTATCAGAATTCTCA | Antisense | 5789 |
| 204533_at | 283 | 277 | 783 | AATGCTCTTTACTTCATGGACTTCC | Antisense | 5790 |
| 204533_at | 394 | 1121 | 791 | TTACTTCATGGACTTCCACTGCCAT | Antisense | 5791 |
| 204533_at | 1139 | 9 | 833 | ATTCTTTCAGTGGCTACCTACATAC | Antisense | 5792 |
| 204533_at | 188 | 509 | 845 | GCTACCTACATACAATTCCAAACAC | Antisense | 5793 |
| 204533_at | 968 | 273 | 1105 | AATGAGGTACTCTCCTGGAAATATT | Antisense | 5794 |
| 205242_at | 195 | 847 | 632 | GGAGTTTGCATTCTTATTCATCAGG | Antisense | 5795 |
| 205242_at | 602 | 591 | 842 | GAAAGACTCAAAAAGCTGCCTGGGA | Antisense | 5796 |
| 205242_at | 551 | 875 | 867 | GGCAGATGGAACTTGAGCCTGTCAA | Antisense | 5797 |
| 205242_at | 516 | 51 | 903 | ATCCATGTAGTAGATATCCTCTGCT | Antisense | 5798 |
| 205242_at | 1011 | 49 | 918 | ATCCTCTGCTTAAAAACTCACTACG | Antisense | 5799 |
| 205242_at | 112 | 285 | 1001 | AATAGCATTCGAAGATCCCCAGACT | Antisense | 5800 |
| 205242_at | 149 | 677 | 1014 | GATCCCCAGACTTCATAGAATACTC | Antisense | 5801 |
| 205242_at | 1073 | 925 | 1061 | TGTACACATGTATCCTTTCACACAT | Antisense | 5802 |
| 205242_at | 988 | 1101 | 1076 | TTTCACACATTTGCCTTGACAAACT | Antisense | 5803 |
| 205242_at | 208 | 949 | 1092 | TGACAAACTTCTTTCACTCACATCT | Antisense | 5804 |
| 205242_at | 350 | 813 | 1151 | GGGGACTCTGGTATCTAATTCTTTA | Antisense | 5805 |
| 205488_at | 857 | 321 | 373 | CAGCCACACGCGAAGGTGACCTTAA | Antisense | 5806 |
| 205488_at | 988 | 619 | 390 | GACCTTAAACTTTTACAGCTGACGG | Antisense | 5807 |
| 205488_at | 301 | 1077 | 435 | TATGTGACTATCCTTCATCTACCTA | Antisense | 5808 |
| 205488_at | 5 | 865 | 483 | GGAACCATGTGCCAAGTTGCAGGGT | Antisense | 5809 |
| 205488_at | 911 | 611 | 515 | GACTCACAATAGTGCATCTTGGTCC | Antisense | 5810 |
| 205488_at | 45 | 523 | 528 | GCATCTTGGTCCGATACTCTGAGAG | Antisense | 5811 |
| 205488_at | 1048 | 965 | 641 | TGCTGGAAGCCTCCGAGGTGGAAGA | Antisense | 5812 |
| 205488_at | 533 | 931 | 697 | TGTTGTGCGAGGGTGTTTTCCGAGG | Antisense | 5813 |
| 205488_at | 741 | 281 | 744 | AATAAATGCGGAGACCCTCGTGGGC | Antisense | 5814 |
| 205488_at | 659 | 405 | 769 | CTGGTGTCTATATTCTTCTCTCAAA | Antisense | 5815 |
| 205488_at | 672 | 373 | 786 | CTCTCAAAGAAACACCTCAACTGGA | Antisense | 5816 |
| 205758_at | 463 | 323 | 1584 | CAGCCCTTGCATTGCAGAGGGGCCC | Antisense | 5817 |
| 205758_at | 1044 | 947 | 1610 | TGAAAGAGGACAGGCTACCCCTTTA | Antisense | 5818 |
| 205758_at | 210 | 217 | 1663 | AAACTAAGGCCCTCTTGAATCTCTG | Antisense | 5819 |
| 205758_at | 1134 | 681 | 1695 | GATACAAACATGTTCCTGGGATCAC | Antisense | 5820 |
| 205758_at | 390 | 123 | 1743 | AGACAATTGTTGGAGAGCCCCTCAC | Antisense | 5821 |
| 205758_at | 338 | 59 | 1804 | ATGAGGCAGACCTGACTCTCTTAAG | Antisense | 5822 |
| 205758_at | 858 | 237 | 1844 | AACTGCTGTCCCAAACATGCACTTC | Antisense | 5823 |
| 205758_at | 774 | 217 | 1856 | AAACATGCACTTCCTTGCTTAAGGT | Antisense | 5824 |
| 205758_at | 546 | 69 | 1881 | ATGGTACAAGCAATGCCTGCCCATT | Antisense | 5825 |
| 205758_at | 190 | 1077 | 1982 | TATGGTGTACATTCTTCCTGATTAT | Antisense | 5826 |
| 205758_at | 1084 | 359 | 1995 | CTTCCTGATTATTTTCTACACATAC | Antisense | 5827 |
| 205890_s_at | 632 | 625 | 195 | GATCTTAAAGCCACGGAGAAGCCTC | Antisense | 5828 |
| 205890_s_at | 247 | 969 | 293 | TGCCCTTGTTTCTTGTGGAGTCAGG | Antisense | 5829 |
| 205890_s_at | 584 | 59 | 320 | ATGAGGCAAAGAGGCACCTCCTCCA | Antisense | 5830 |
| 205890_s_at | 578 | 561 | 350 | GAAGGTCCAGCTCAGTGGCACAAGT | Antisense | 5831 |
| 205890_s_at | 767 | 615 | 396 | GACGGGTATAATCCCTGAGACCCAG | Antisense | 5832 |
| 205890_s_at | 615 | 619 | 414 | GACCCAGATTGTGACTTGCAATGGA | Antisense | 5833 |
| 205890_s_at | 783 | 113 | 481 | AGAAAGGGCAACTTACTCTTCCTGG | Antisense | 5834 |
| 205890_s_at | 67 | 173 | 495 | ACTCTTCCTGGCATCTTATTGTATT | Antisense | 5835 |
| 205890_s_at | 814 | 279 | 578 | AATCTCTTACTCAACGAACACATCT | Antisense | 5836 |
| 205890_s_at | 573 | 349 | 598 | CATCTTCTGATGATTTCCCAAAATT | Antisense | 5837 |
| 205890_s_at | 415 | 7 | 678 | ATTGCCCAACTCTATGTTTCTTTGA | Antisense | 5838 |
| 206134_at | 437 | 367 | 1600 | CTTTCTATATTGTTATCAGTCCAGG | Antisense | 5839 |
| 206134_at | 424 | 607 | 1650 | GACATTGGCTCTTTGTTTAGGCCTA | Antisense | 5840 |
| 206134_at | 26 | 551 | 1725 | GAATTTGTGACTTAGTTCTGCCCTT | Antisense | 5841 |
| 206134_at | 512 | 591 | 1762 | GAAAGCAGTCTTCCATCAAATCACC | Antisense | 5842 |
| 206134_at | 398 | 275 | 1791 | AATGCACGGCTAAACTATTCAGAGT | Antisense | 5843 |
| 206134_at | 1079 | 259 | 1841 | AAGTACTATGCTTTAATGCTTCTTT | Antisense | 5844 |
| 206134_at | 443 | 517 | 1858 | GCTTCTTTCATCTTACTAGTATGGC | Antisense | 5845 |
| 206134_at | 791 | 1077 | 1953 | TATGTTATTCCTCTGTGTTCACTTC | Antisense | 5846 |
| 206134_at | 539 | 177 | 1973 | ACTTCGCCTTGCTCTTGAAAGTGCA | Antisense | 5847 |
| 206134_at | 1134 | 721 | 2055 | GTATATCCTATACACACATCTCCTT | Antisense | 5848 |
| 206134_at | 103 | 943 | 2104 | TGAGAAGTCTACATTGCTTACATTT | Antisense | 5849 |
| 206666_at | 531 | 211 | 471 | AAACCTCTCTTAGATCTGGAACCAA | Antisense | 5850 |
| 206666_at | 1105 | 147 | 517 | AGCCACCGATCCAGATTCATTAAGA | Antisense | 5851 |
| 206666_at | 321 | 1131 | 532 | TTCATTAAGACCTTCTGACACCCTG | Antisense | 5852 |
| 206666_at | 404 | 191 | 549 | ACACCCTGCGAGAAGTCACTGTTAC | Antisense | 5853 |
| 206666_at | 594 | 813 | 696 | GGGGCCCCTTGATCTGTAAAGGTGT | Antisense | 5854 |
| 206666_at | 586 | 207 | 713 | AAAGGTGTCTTCCACGCTATAGTCT | Antisense | 5855 |
| 206666_at | 673 | 785 | 753 | GTGGTGTTGCCACAAAGCCTGGAAT | Antisense | 5856 |
| 206666_at | 972 | 867 | 773 | GGAATCTACACCCTGTTAACCAAGA | Antisense | 5857 |
| 206666_at | 335 | 905 | 809 | TGGATCAAAAGCAACCTTGTCCCGC | Antisense | 5858 |
| 206666_at | 590 | 757 | 827 | GTCCCGCCTCATACAAATTAAGTTA | Antisense | 5859 |
| 206666_at | 448 | 1075 | 935 | TATGGGGTCCATTTTTGCACTTGTA | Antisense | 5860 |
| 207651_at | 757 | 1147 | 828 | TTGCCTTGTAATTCGACAGCTCTAC | Antisense | 5861 |
| 207651_at | 1133 | 175 | 906 | ACTTTTAGTGACCACGGGCTACATC | Antisense | 5862 |
| 207651_at | 103 | 795 | 921 | GGGCTACATCATATGCTTTGTTCCT | Antisense | 5863 |
| 207651_at | 23 | 31 | 968 | ATACCCTCAGCCAGACAGAAGTCAT | Antisense | 5864 |
| 207651_at | 997 | 563 | 985 | GAAGTCATAACTGATTGCTCAACCA | Antisense | 5865 |
| 207651_at | 1084 | 961 | 1000 | TGCTCAACCAGGATTTCACTCTTCA | Antisense | 5866 |
| 207651_at | 572 | 245 | 1025 | AAGCCAAAGAGGCTACACTGCTCCT | Antisense | 5867 |
| 207651_at | 1026 | 961 | 1066 | TGCTTTGATCCTATCCTGTACTATC | Antisense | 5868 |
| 207651_at | 257 | 211 | 1099 | AAAGCATTCCGCTCAAAGGTCACTG | Antisense | 5869 |
| 207651_at | 1005 | 1027 | 1118 | TCACTGAGACTTTTGCCTCACCTAA | Antisense | 5870 |
| 207651_at | 800 | 959 | 1208 | TGCTACCAATTCTGGCCTTACTGGA | Antisense | 5871 |
| 209606_at | 595 | 493 | 1164 | GAATTGCAAAACTGACATCCCATTT | Antisense | 5872 |
| 209606_at | 140 | 169 | 1174 | ACTGACATCCCATTTCACAGCAATA | Antisense | 5873 |
| 209606_at | 1008 | 309 | 1189 | CACAGCAATAGTGACCTTTATTTAA | Antisense | 5874 |
| 209606_at | 778 | 697 | 1217 | GTTGTGTTATAGTTTATGCTTCTTA | Antisense | 5875 |
| 209606_at | 422 | 1099 | 1249 | TTTCAACCTAAACAGCCAATTTCTA | Antisense | 5876 |
| 209606_at | 735 | 189 | 1260 | ACAGCCAATTTCTAAGCAGACAGGA | Antisense | 5877 |
| 209606_at | 240 | 1087 | 1369 | TATTTTCTAGATTATCCCTGTGAAT | Antisense | 5878 |
| 209606_at | 292 | 201 | 1485 | AAATCTTAGGTTTGCTTATGCCCAG | Antisense | 5879 |
| 209606_at | 775 | 499 | 1590 | GCTGTTTCTCACATCTATAGTGACA | Antisense | 5880 |
| 209606_at | 195 | 25 | 1639 | ATAGGAGGGGTTAAGGTTCATGAGA | Antisense | 5881 |
| 209606_at | 790 | 823 | 1679 | GGTCTGAGATGGGTGCTGCAAAGAT | Antisense | 5882 |
| 209795_at | 451 | 1071 | 1143 | TAGTCTAATTGAATCCCTTAAACTC | Antisense | 5883 |
| 209795_at | 691 | 67 | 1273 | ATGGGATGATCGTGTATTTATTTTT | Antisense | 5884 |
| 209795_at | 665 | 1107 | 1294 | TTTTTTACTTCCTCAGCTGTAGACA | Antisense | 5885 |
| 209795_at | 1000 | 177 | 1300 | ACTTCCTCAGCTGTAGACAGGTCCT | Antisense | 5886 |
| 209795_at | 563 | 603 | 1315 | GACAGGTCCTTTTCGATGGTACATA | Antisense | 5887 |
| 209795_at | 188 | 907 | 1331 | TGGTACATATTTCTTTGCCTTTATA | Antisense | 5888 |
| 209795_at | 302 | 1083 | 1352 | TATAATCTTTTATACAGTGTCTTAC | Antisense | 5889 |
| 209795_at | 1015 | 773 | 1450 | GTGATGTGGCAAATCTCTATTAGGA | Antisense | 5890 |
| 209795_at | 970 | 21 | 1476 | ATATTCTGTAATCTTCAGACCTAGA | Antisense | 5891 |
| 209795_at | 859 | 83 | 1520 | AGGTTTGTGACTTTCCTAAATCAAT | Antisense | 5892 |
| 209795_at | 232 | 1059 | 1550 | TACGTGCAATACTTCAATACTTCAT | Antisense | 5893 |
| 210915_x_at | 1077 | 205 | 516 | AAAGGCCACACTGGTGTGCCTGGCC | Antisense | 5894 |
| 210915_x_at | 1116 | 945 | 651 | TGACTCCAGATACTGCCTGAGCAGC | Antisense | 5895 |
| 210915_x_at | 928 | 985 | 722 | TCCGCTGTCAAGTCCAGTTCTACGG | Antisense | 5896 |
| 210915_x_at | 495 | 419 | 807 | CGCCGAGGCCTGGGGTAGAGCAGAC | Antisense | 5897 |
| 210915_x_at | 185 | 1067 | 822 | TAGAGCAGACTGTGGCTTTACCTCG | Antisense | 5898 |
| 210915_x_at | 1098 | 461 | 875 | CCACCATCCTCTATGAGATCCTGCT | Antisense | 5899 |
| 210915_x_at | 577 | 109 | 890 | AGATCCTGCTAGGGAAGGCCACCAT | Antisense | 5900 |
| 210915_x_at | 857 | 269 | 904 | AAGGCCACCATGTATGCTGTGCTGG | Antisense | 5901 |
| 210915_x_at | 145 | 419 | 933 | CGCCCTTGTGTTGATGGCCATGGTC | Antisense | 5902 |
| 210915_x_at | 224 | 137 | 1001 | AGCTTCTAACCCGTCATGGTTTCAA | Antisense | 5903 |
| 210915_x_at | 864 | 293 | 1023 | CAATACACATTCTTCTTTTGCCAGC | Antisense | 5904 |
| 210972_x_at | 474 | 861 | 514 | GGAACAAGACTTCAGGTCACGCTCG | Antisense | 5905 |
| 210972_x_at | 393 | 1021 | 525 | TCAGGTCACGCTCGATATCCAGAAC | Antisense | 5906 |
| 210972_x_at | 111 | 949 | 591 | TGACAAGTCTGTCTGCCTATTCACC | Antisense | 5907 |
| 210972_x_at | 583 | 633 | 711 | GAGCAACAGTGCTGTGGCCTGGAGC | Antisense | 5908 |
| 210972_x_at | 60 | 525 | 751 | GCATGTGCAAACGCCTTCAACAACA | Antisense | 5909 |
| 210972_x_at | 317 | 987 | 800 | TCCCCAGCCCAGAAAGTTCCTGTGA | Antisense | 5910 |
| 210972_x_at | 461 | 701 | 815 | GTTCCTGTGATGTCAAGCTGGTCGA | Antisense | 5911 |
| 210972_x_at | 1124 | 335 | 884 | CAGTGATTGGGTTCCGAATCCTCCT | Antisense | 5912 |
| 210972_x_at | 920 | 263 | 914 | AAGTGGCCGGGTTTAATCTGCTCAT | Antisense | 5913 |
| 210972_x_at | 1017 | 497 | 948 | GCTGTGGTCCAGCTGAGATCTGCAA | Antisense | 5914 |
| 210972_x_at | 49 | 695 | 973 | GATTGTAAGACAGCCTGTGCTCCCT | Antisense | 5915 |
| 211796_s_at | 480 | 475 | 459 | GCCATCAGAAGCAGAGATCTCCCAC | Antisense | 5916 |
| 211796_s_at | 1017 | 445 | 618 | CCTCAATGACTCCAGATACTGCCTG | Antisense | 5917 |
| 211796_s_at | 464 | 295 | 621 | CAATGACTCCAGATACTGCCTGAGC | Antisense | 5918 |
| 211796_s_at | 927 | 985 | 695 | TCCGCTGTCAAGTCCAGTTCTACGG | Antisense | 5919 |
| 211796_s_at | 588 | 401 | 699 | CTGTCAAGTCCAGTTCTACGGGCTC | Antisense | 5920 |
| 211796_s_at | 538 | 331 | 709 | CAGTTCTACGGGCTCTCGGAGAATG | Antisense | 5921 |
| 211796_s_at | 612 | 995 | 724 | TCGGAGAATGACGAGTGGACCCAGG | Antisense | 5922 |
| 211796_s_at | 969 | 253 | 730 | AATGACGAGTGGACCCAGGATAGGG | Antisense | 5923 |
| 211796_s_at | 396 | 437 | 782 | CCGAGGCCTGGGGTAGAGCAGACTG | Antisense | 5924 |
| 211796_s_at | 704 | 439 | 788 | CCTGGGGTAGAGCAGACTGTGGCTT | Antisense | 5925 |
| 211796_s_at | 1147 | 937 | 917 | TGATGGCCATGGTCAAGAGAAAGGA | Antisense | 5926 |
| 212592_at | 641 | 723 | 859 | GTATCAAAATCTTCCAATTATCATG | Antisense | 5927 |
| 212592_at | 937 | 39 | 867 | ATCTTCCAATTATCATGCTCACCTG | Antisense | 5928 |
| 212592_at | 25 | 975 | 871 | TCCAATTATCATGCTCACCTGAAAG | Antisense | 5929 |
| 212592_at | 92 | 509 | 883 | GCTCACCTGAAAGAGGTATGCTCTC | Antisense | 5930 |
| 212592_at | 880 | 657 | 895 | GAGGTATGCTCTCTTAGGAATACAG | Antisense | 5931 |
| 212592_at | 700 | 1073 | 909 | TAGGAATACAGTTTCTAGCATTAAA | Antisense | 5932 |
| 212592_at | 187 | 1035 | 985 | TAATAAAATGTTCCTCGCATTCCCC | Antisense | 5933 |
| 212592_at | 374 | 103 | 1088 | AGTTTCACTGTGTAGAGAACATATA | Antisense | 5934 |
| 212592_at | 317 | 27 | 1122 | ATAGGTCAATTATATGTCTCCATTA | Antisense | 5935 |
| 212592_at | 790 | 217 | 1164 | AAACATGTTCTAGAACTAGTTACAA | Antisense | 5936 |
| 212592_at | 384 | 199 | 1248 | AAATATATTTGGACATAACAGACTT | Antisense | 5937 |
| 212999_x_at | 68 | 315 | 651 | CACTGCAGAATGAAGGAACATCCCT | Antisense | 5938 |
| 212999_x_at | 934 | 267 | 663 | AAGGAACATCCCTTGAGGTGACCCA | Antisense | 5939 |
| 212999_x_at | 96 | 423 | 684 | CCCAGCCAACCTGTGGCCAGAAGGA | Antisense | 5940 |
| 212999_x_at | 241 | 731 | 714 | GTACCTTGAAAAGACACTGAAAGCA | Antisense | 5941 |
| 212999_x_at | 138 | 737 | 753 | GTAAGGGTGGGCAGAGGAGGTAGAA | Antisense | 5942 |
| 212999_x_at | 969 | 35 | 779 | ATCAATTCAATTGTCGCATCATTCA | Antisense | 5943 |
| 212999_x_at | 172 | 293 | 787 | AATTGTCGCATCATTCATGGTTCTT | Antisense | 5944 |
| 212999_x_at | 43 | 757 | 791 | GTCGCATCATTCATGGTTCTTTAAT | Antisense | 5945 |
| 212999_x_at | 987 | 831 | 805 | GGTTCTTTAATATTGATGCTCAGTG | Antisense | 5946 |
| 212999_x_at | 201 | 453 | 922 | CCTTGGTCACTGGTGTTTCAAACAT | Antisense | 5947 |
| 212999_x_at | 299 | 1125 | 984 | TTAAGAAAGCATAACCAGCAATAAA | Antisense | 5948 |
| 213193_x_at | 1117 | 945 | 664 | TGACTCCAGATACTGCCTGAGCAGC | Antisense | 5949 |
| 213193_x_at | 926 | 985 | 735 | TCCGCTGTCAAGTCCAGTTCTACGG | Antisense | 5950 |
| 213193_x_at | 186 | 1067 | 835 | TAGAGCAGACTGTGGCTTTACCTCG | Antisense | 5951 |
| 213193_x_at | 1097 | 461 | 888 | CCACCATCCTCTATGAGATCCTGCT | Antisense | 5952 |
| 213193_x_at | 254 | 643 | 902 | GAGATCCTGCTAGGGAAGGCCACCC | Antisense | 5953 |
| 213193_x_at | 74 | 319 | 922 | CACCCTGTATGCTGTGCTGGTCAGC | Antisense | 5954 |
| 213193_x_at | 863 | 293 | 1036 | CAATACACATTCTTCTTTTGCCAGC | Antisense | 5955 |
| 213193_x_at | 1099 | 471 | 1055 | GCCAGCGCTTCTGAAGAGCTGCTCT | Antisense | 5956 |
| 213193_x_at | 522 | 155 | 1081 | ACCTCTCTGCATCCCAATAGATATC | Antisense | 5957 |
| 213193_x_at | 717 | 283 | 1096 | AATAGATATCCCCCTATGTGCATGC | Antisense | 5958 |
| 213193_x_at | 782 | 811 | 1158 | GGGGGACCTTAGCATGCCTAAGTGA | Antisense | 5959 |
| 213539_at | 163 | 799 | 265 | GGGAACACTGCTCTCAGACATTACA | Antisense | 5960 |
| 213539_at | 711 | 613 | 291 | GACTGGACCTGGGAAAACGCATCCT | Antisense | 5961 |
| 213539_at | 701 | 417 | 308 | CGCATCCTGGACCCACGAGGAATAT | Antisense | 5962 |
| 213539_at | 383 | 553 | 368 | GAATCTACCGTGCAAGTTCATTATC | Antisense | 5963 |
| 213539_at | 835 | 781 | 408 | GTGTGGAGCTGGATCCAGCCACCGT | Antisense | 5964 |
| 213539_at | 610 | 119 | 507 | AGACTGGAAGGCTGTCTGGGGCTGC | Antisense | 5965 |
| 213539_at | 108 | 971 | 529 | TGCCGACACACAAGCTCTGTTGAGG | Antisense | 5966 |
| 213539_at | 964 | 867 | 552 | GGAATGACCAGGTCTATCAGCCCCT | Antisense | 5967 |
| 213539_at | 659 | 145 | 570 | AGCCCCTCCGAGATCGAGATGATGC | Antisense | 5968 |
| 213539_at | 1113 | 961 | 592 | TGCTCAGTACAGCCACCTTGGAGGA | Antisense | 5969 |
| 213539_at | 892 | 569 | 659 | GAAGCAGCCATTACCAACTGTACCT | Antisense | 5970 |
| 214617_at | 814 | 465 | 1921 | CCAACGCAAATTCGCAAACTTTCTT | Antisense | 5971 |
| 214617_at | 318 | 507 | 1990 | GCTCATCGGCTATCGTTAGTGCTAG | Antisense | 5972 |
| 214617_at | 173 | 963 | 2009 | TGCTAGTGGATTTTACATGTGGCCC | Antisense | 5973 |
| 214617_at | 927 | 105 | 2073 | AGATTGGATACGCATCAGACAGATG | Antisense | 5974 |
| 214617_at | 1040 | 655 | 2127 | GAGGTGGCTGGGTTTACACGCTAAT | Antisense | 5975 |
| 214617_at | 220 | 805 | 2136 | GGGTTTACACGCTAATCCCGATTCA | Antisense | 5976 |
| 214617_at | 736 | 529 | 2203 | GCAGTCACAGCTACACAGATCACAG | Antisense | 5977 |
| 214617_at | 52 | 477 | 2331 | GCCTTCATTGCCTTAACTGGAGCCC | Antisense | 5978 |
| 214617_at | 212 | 295 | 2389 | CAATGCTTTGCCCCTGAGACAAATG | Antisense | 5979 |
| 214617_at | 1085 | 597 | 2406 | GACAAATGGAGGCCTGTCCTGACCT | Antisense | 5980 |
| 214617_at | 566 | 157 | 2427 | ACCTGTCTCACCATGTACATAGCTT | Antisense | 5981 |
| 216920_s_at | 416 | 313 | 881 | CACTACTGCTGCAGCTCACAAACAC | Antisense | 5982 |
| 216920_s_at | 585 | 509 | 894 | GCTCACAAACACCTCTGCATATTAC | Antisense | 5983 |
| 216920_s_at | 504 | 1001 | 907 | TCTGCATATTACATGTACCTCCTCC | Antisense | 5984 |
| 216920_s_at | 513 | 783 | 944 | GTGTGGTCTATTTTGCCATCATCAC | Antisense | 5985 |
| 216920_s_at | 940 | 891 | 1115 | GGCCATTTCAGTTCTCATGTGTGTA | Antisense | 5986 |
| 216920_s_at | 978 | 829 | 1161 | GGTTTTCAAACCAGTGGGCACACAG | Antisense | 5987 |
| 216920_s_at | 584 | 989 | 1323 | TCCCTCTCTTAGTGTTCTTTAATCA | Antisense | 5988 |
| 216920_s_at | 126 | 35 | 1349 | ATAACTGCCTGGAAGCCTTTCATTT | Antisense | 5989 |
| 216920_s_at | 170 | 897 | 1358 | TGGAAGCCTTTCATTTTACACGCCC | Antisense | 5990 |
| 216920_s_at | 1055 | 417 | 1378 | CGCCCTGAAGCAGTCTTCTTTGCTA | Antisense | 5991 |
| 216920_s_at | 1041 | 1115 | 1409 | TTATGTGGTGTGTTTTTCCGTAATA | Antisense | 5992 |
| 217147_s_at | 440 | 1003 | 1036 | TCTCCTTTCTCACCAATGGGCAATA | Antisense | 5993 |
| 217147_s_at | 398 | 981 | 1038 | TCCTTTCTCACCAATGGGCAATAGC | Antisense | 5994 |
| 217147_s_at | 256 | 1087 | 1149 | TATTTTTCCCAAAGATAGCTTTACT | Antisense | 5995 |
| 217147_s_at | 565 | 743 | 1186 | GTCAAATTAATGCATGCTCCTTACA | Antisense | 5996 |
| 217147_s_at | 863 | 275 | 1194 | AATGCATGCTCCTTACAACAAACAA | Antisense | 5997 |
| 217147_s_at | 1061 | 53 | 1199 | ATGCTCCTTACAACAAACAAATATC | Antisense | 5998 |
| 217147_s_at | 559 | 647 | 1229 | GAGTTTAGGAATTCTACTAGCCAGA | Antisense | 5999 |
| 217147_s_at | 368 | 173 | 1244 | ACTAGCCAGAGATAGTCACTTGGAG | Antisense | 6000 |
| 217147_s_at | 635 | 685 | 1254 | GATAGTCACTTGGAGAAACTTTCTA | Antisense | 6001 |
| 217147_s_at | 69 | 589 | 1268 | GAAACTTTCTATATATCCTTCTAAA | Antisense | 6002 |
| 217147_s_at | 102 | 319 | 1480 | CACCCCACTGATAACCAAATCACAG | Antisense | 6003 |
| 222838_at | 228 | 229 | 2352 | AACACCTGTGCTAGGTCAGTCTGGC | Antisense | 6004 |
| 222838_at | 971 | 509 | 2361 | GCTAGGTCAGTCTGGCACGTAAGAT | Antisense | 6005 |
| 222838_at | 805 | 755 | 2370 | GTCTGGCACGTAAGATGAACATCCC | Antisense | 6006 |
| 222838_at | 1054 | 1039 | 2380 | TAAGATGAACATCCCTACCAACACA | Antisense | 6007 |
| 222838_at | 728 | 629 | 2405 | GAGCTCACCATCTCTTATACTTAAG | Antisense | 6008 |
| 222838_at | 821 | 61 | 2473 | ATGTTCCCTGACACATATCTTGAAT | Antisense | 6009 |
| 222838_at | 479 | 179 | 2485 | ACATATCTTGAATGGAGACCTCCCT | Antisense | 6010 |
| 222838_at | 379 | 119 | 2500 | AGACCTCCCTACCAAGTGATGAAAG | Antisense | 6011 |
| 222838_at | 520 | 805 | 2565 | GGGATTGAGGATTATCTTCTCTCAG | Antisense | 6012 |
| 222838_at | 382 | 1013 | 2579 | TCTTCTCTCAGAAAGGCATTGTGAA | Antisense | 6013 |
| 222838_at | 797 | 447 | 2626 | CCTACTGCAAAACCCTATTGTAGTA | Antisense | 6014 |
| 222895_s_at | 1120 | 703 | 3909 | GTTCTCTCATTTAATGCACACTGAT | Antisense | 6015 |
| 222895_s_at | 361 | 941 | 4003 | TGAGTTCCATCAGAAGGTGCCCAGC | Antisense | 6016 |
| 222895_s_at | 203 | 1107 | 4051 | TTTTTTCCTATCTCATTAGGTTGGA | Antisense | 6017 |
| 222895_s_at | 216 | 9 | 4114 | ATTCTGTTGACCCGCACTTTAAAGC | Antisense | 6018 |
| 222895_s_at | 821 | 5 | 4178 | ATTGCAGCATATTCTTCTCTTTGGC | Antisense | 6019 |
| 222895_s_at | 1015 | 9 | 4188 | ATTCTTCTCTTTGGCCCAGAGGTGG | Antisense | 6020 |
| 222895_s_at | 758 | 651 | 4240 | GAGTGTCAGTATTGCTAAGCGTGGC | Antisense | 6021 |
| 222895_s_at | 1024 | 493 | 4258 | GCGTGGCATTCACAATACTGGCACT | Antisense | 6022 |
| 222895_s_at | 788 | 713 | 4318 | GTTTTTCTACTGCCATTCAATTTGA | Antisense | 6023 |
| 222895_s_at | 687 | 773 | 4344 | GTGAGTGCCTTGAAAACTGATCTTC | Antisense | 6024 |
| 222895_s_at | 948 | 41 | 4363 | ATCTTCCTATTTGAGTCTCTTGAGA | Antisense | 6025 |
| 226218_at | 938 | 375 | 979 | CTAATTGGTTCTGCCCAATCTCCTT | Antisense | 6026 |
| 226218_at | 739 | 943 | 1126 | TGACTGGGTCTAGGGCACCCAGGCT | Antisense | 6027 |
| 226218_at | 810 | 691 | 1151 | GATTCAGCTGATTTCCTACCAGCCT | Antisense | 6028 |
| 226218_at | 730 | 1075 | 1229 | TATGGGCTGTTCAGAGGTGCACACC | Antisense | 6029 |
| 226218_at | 1063 | 249 | 1283 | AAGAGACTTGGTACGGGCCAGGAAG | Antisense | 6030 |
| 226218_at | 819 | 551 | 1307 | GAATATGTGGCAGAGCTCCTGGAAA | Antisense | 6031 |
| 226218_at | 949 | 1073 | 1342 | TAGGTGGCATTTTTGTCAGCTCTGT | Antisense | 6032 |
| 226218_at | 721 | 745 | 1356 | GTCAGCTCTGTGGTTTATTGTTGGG | Antisense | 6033 |
| 226218_at | 938 | 199 | 1392 | AAATATCCATTGTTCACTACAGTGA | Antisense | 6034 |
| 226218_at | 984 | 157 | 1431 | ACCGTGTACTATCCACATGCATTAC | Antisense | 6035 |
| 226218_at | 64 | 13 | 1451 | ATTACAAACATTTCGCAGAGCTGCT | Antisense | 6036 |
| 227346_at | 745 | 961 | 407 | TGCTTTTGGTGCGATGGCACTCACT | Antisense | 6037 |
| 227346_at | 845 | 547 | 460 | GCAATATTGTTTCCAATACTTTCTA | Antisense | 6038 |
| 227346_at | 374 | 783 | 507 | GTGTGGTGATTGTTCAGGTCGAATC | Antisense | 6039 |
| 227346_at | 83 | 85 | 522 | AGGTCGAATCTGTTGTATCCAGTAC | Antisense | 6040 |
| 227346_at | 359 | 51 | 538 | ATCCAGTACAGCTTTAGGTCTTCAG | Antisense | 6041 |
| 227346_at | 120 | 393 | 563 | CTGCCCTTCTGGCGAGTACATGCAC | Antisense | 6042 |
| 227346_at | 211 | 959 | 606 | TGCAGTCATATTTCCAGTCTGCCTC | Antisense | 6043 |
| 227346_at | 628 | 977 | 618 | TCCAGTCTGCCTCTATGATGATGTT | Antisense | 6044 |
| 227346_at | 207 | 583 | 664 | GAACAAGGGATGTACCACTGGAGGA | Antisense | 6045 |
| 227346_at | 682 | 647 | 692 | GAGTATCCTTTTGTACACATTTTGA | Antisense | 6046 |
| 227346_at | 65 | 277 | 717 | AATGCTTCTTCTGTAGTGATAGAAC | Antisense | 6047 |
| 228362_s_at | 397 | 733 | 22 | GTACTGGCCCTTCGGATTGAAAGTA | Antisense | 6048 |
| 228362_s_at | 842 | 345 | 76 | CATGCTTGGAGTGTTATATTCTTTT | Antisense | 6049 |
| 228362_s_at | 383 | 21 | 91 | ATATTCTTTTGGATGCGAGCCCTCA | Antisense | 6050 |
| 228362_s_at | 1068 | 491 | 105 | GCGAGCCCTCAAAGAAACATTTAAT | Antisense | 6051 |
| 228362_s_at | 564 | 1125 | 125 | TTAATATTCTCTTTTGCCAATTCAG | Antisense | 6052 |
| 228362_s_at | 1118 | 467 | 140 | GCCAATTCAGTTGCATGCTCTGTGG | Antisense | 6053 |
| 228362_s_at | 885 | 523 | 152 | GCATGCTCTGTGGCTTTACTTTTAA | Antisense | 6054 |
| 228362_s_at | 908 | 1121 | 167 | TTACTTTTAAGGATCTGCTGCTCCT | Antisense | 6055 |
| 228362_s_at | 444 | 27 | 199 | ATAGATTTTCCAGAATTTCAGCTGC | Antisense | 6056 |
| 228362_s_at | 191 | 239 | 232 | AACTGGAGATAGGCATCGGGTGACA | Antisense | 6057 |
| 228362_s_at | 893 | 237 | 286 | AACTGCTATCAAGATCCAGCCCAAC | Antisense | 6058 |
| 229152_at | 192 | 953 | 20 | TGAAGAAAGTTCTCCTCCTGATCAC | Antisense | 6059 |
| 229152_at | 858 | 387 | 34 | CTCCTGATCACAGCCATCTTGGCAG | Antisense | 6060 |
| 229152_at | 299 | 455 | 47 | CCATCTTGGCAGTGGCTGTTGGTTT | Antisense | 6061 |
| 229152_at | 343 | 603 | 115 | GACAGCGATGAATTAGCTTCAGGGT | Antisense | 6062 |
| 229152_at | 793 | 975 | 177 | TCCACCAATTCCATTTCCAAGATTT | Antisense | 6063 |
| 229152_at | 136 | 257 | 195 | AAGATTTCCATGGTTTAGACGTAAT | Antisense | 6064 |
| 229152_at | 502 | 1067 | 210 | TAGACGTAATTTTCCTATTCCAATA | Antisense | 6065 |
| 229152_at | 194 | 369 | 224 | CTATTCCAATACCTGAATCTGCCCC | Antisense | 6066 |
| 229152_at | 124 | 209 | 287 | AAAGTCACGATAAACCTGGTCACCT | Antisense | 6067 |
| 229152_at | 1068 | 199 | 382 | AAATAGCACACAGCATTCTCTAGTC | Antisense | 6068 |
| 229152_at | 142 | 25 | 408 | ATATCTTTAGTGATCTTCTTTAATA | Antisense | 6069 |
| 231577_s_at | 400 | 863 | 256 | GGAACAGGAGCAACTACTAAAAGAG | Antisense | 6070 |
| 231577_s_at | 96 | 681 | 316 | GATACAGGATCTCCAGACGAAAATG | Antisense | 6071 |
| 231577_s_at | 274 | 639 | 340 | GAGACGACGAAAGGCATGTACCATA | Antisense | 6072 |
| 231577_s_at | 313 | 877 | 352 | GGCATGTACCATAAGCTAAAGACCA | Antisense | 6073 |
| 231577_s_at | 1053 | 373 | 367 | CTAAAGACCAGAGCCTTCCTGTCAC | Antisense | 6074 |
| 231577_s_at | 227 | 925 | 386 | TGTCACCCCTAACCAAGGCATAATT | Antisense | 6075 |
| 231577_s_at | 43 | 551 | 423 | GAATTTGGAACAAGCGTCACTACAT | Antisense | 6076 |
| 231577_s_at | 1095 | 861 | 429 | GGAACAAGCGTCACTACATTTGATA | Antisense | 6077 |
| 231577_s_at | 249 | 247 | 434 | AAGCGTCACTACATTTGATAATAAT | Antisense | 6078 |
| 231577_s_at | 616 | 209 | 481 | AAAGTTTATAAAGGCATGTGGTACA | Antisense | 6079 |
| 231577_s_at | 216 | 877 | 493 | GGCATGTGGTACAATGATCAAAATC | Antisense | 6080 |
| 236280_at | 531 | 223 | 245 | AAAAGATCTCTCACTGGGAAAAGAA | Antisense | 6081 |
| 236280_at | 235 | 713 | 301 | GTTTTCCTTGTACTTTATTAATCTG | Antisense | 6082 |
| 236280_at | 647 | 553 | 325 | GAATCTAATGGCACTTCCTTACGAG | Antisense | 6083 |
| 236280_at | 902 | 363 | 342 | CTTACGAGGGTTTTCAGATGTGCTT | Antisense | 6084 |
| 236280_at | 1064 | 57 | 391 | ATGACTACACAGACAGTCCTACTCT | Antisense | 6085 |
| 236280_at | 326 | 605 | 402 | GACAGTCCTACTCTGAGGAGATGAC | Antisense | 6086 |
| 236280_at | 501 | 567 | 431 | GAAGAAACCCATTTGGAACTACACA | Antisense | 6087 |
| 236280_at | 229 | 863 | 479 | GGAACTGCAATCCTCAAGAGTCACA | Antisense | 6088 |
| 236280_at | 736 | 255 | 494 | AAGAGTCACACTTCATATTCCTTCC | Antisense | 6089 |
| 236280_at | 815 | 1145 | 511 | TTCCTTCCTTTCAAGTGGTTGATAA | Antisense | 6090 |
| 236280_at | 369 | 231 | 585 | AACATGCTTTTTATTTTGACTACCA | Antisense | 6091 |

1-19. (canceled)

20. A method for classifying a cancer patient as a responder or non-responder to a cancer immunotherapy comprising the steps of:
(a) obtaining a patient-derived sample and measuring the expression levels of a profile of gene products comprising the gene products of the genes recognised by the probe sets listed in Table 1 (SEQ ID NOS: 1-18); and
(b) classifying the patient as a responder or non-responder to a cancer immunotherapy based on the expression levels.

21. The method of claim 20, wherein the classification step is performed by reference to a standard.

22. The method of claim 21, wherein the standard is a patient-derived sample from a patient having a known clinical outcome.

23. A method of treating a patient comprising the steps of:
(a) obtaining a classification of the patient as a responder or non-responder to a cancer immunotherapy based on the expression levels of a profile of gene products comprising the gene products of the genes recognised by the probe sets listed in Table 1 (SEQ ID NOS: 1-18); and
(b) selecting the patient for at least one administration of an appropriate immunotherapeutic if the patient is characterized as a responder to the immunotherapeutic.

24. The method of claim 23, wherein the immunotherapeutic further comprises an adjuvant.

25. The method of claim 24, wherein the immunotherapeutic further comprises a MAGE antigen.
